SEQUENCE LISTING (600 sequences)

SEQ ID NO: 1 - DNA, 1536 bp, Staphylococcus aureus
atgactttacaaatacatacagggggtattaatttgaaaaagaaaaacatttattcaattcgtaaactaggtgtaggtattgcatctgtaactttaggtacattacttatatctggtggcgtaacacctgctgcaaatgctgcgcaacacgatgaagctcaacaaaatgctttttatcaagtgttaaatatgcctaacttaaacgctgatcaacgtaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaatgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcttaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagaggaagacaacaaaaaacctggtaaagaagacggcaacaagcctggtaaagaagacaacaaaaaacctggtaaagaagacggcaacaagcctggtaaagaagacaacaacaaacctggcaaagaagacggcaacaagcctggtaaagaagacaacaacaagcctggtaaagaagacggcaacaagcctggtaaagaagacggcaacaaacctggtaaagaagacggcaacggagtacatgtcgttaaacctggtgatacagtaaatgacattgcaaaagcaaacggcactactgctgacaaaattgctgcagataacaaattagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataa

SEQ ID NO: 2 - DNA, 993 bp, Staphylococcus aureus
atgaataaagtaattaaaatgcttgttgttacgcttgctttcctacttgttttagcaggatgtagtgggaattcaaataaacaatcatctgataacaaagataaggaaacaacttcaattaaacatgcaatgggtacaactgaaattaaagggaaaccaaagcgtgttgttacgctatatcaaggtgccactgacgtcgctgtatctttaggtgttaaacctgtaggtgctgtagaatcatggacacaaaaaccgaaattcgaatacataaaaaatgatttaaaagatactaagattgtaggtcaagaacctgcacctaacttagaggaaatctctaaattaaaaccggacttaattgtcgcgtcaaaagttagaaatgaaaaagtttacgatcaattatctaaaatcgcaccaacagtttctactgatacagttttcaaattcaaagatacaactaagttaatggggaaagctttagggaaagaaaaagaagctgaagatttacttaaaaagtacgatgataaagtagctgcattccaaaaagatgcaaaagcaaagtataaagatgcatggccattgaaagcttcagttgttaacttccgtgctgatcatacaagaatttatgctggtggatatgctggtgaaatcttaaatgatttaggattcaaacgtaataaagacttacaaaaacaagttgataatggtaaagatattatccaacttacatctaaagaaagcattccattaatgaacgctgatcatatttttgtagtaaaatcagatccaaatgcgaaagatgctgcattagttaaaaagactgaaagcgaatggacttcaagtaaagagtggaaaaatttagacgcagttaaaaacaaccaagtatctgatgatttagatgaaatcacttggaacttagctggcggatataaatcttcattaaaacttattgacgatttatatgaaaagttaaatattgaaaaacaatcaaaataa

SEQ ID NO: 3 - DNA, 1014 bp, Staphylococcus aureus
atgataatgattatcattaatttaaagggagaaaaatttgtaatgaagtatttattaaagggaaatattttgcttctattactaatattgttgacaattatttcgttgttcataggtgtgagtgaactatcaattaaagatttactacatttaactgaatcacagcggaatattttattctcaagccgaataccaaggacgatgagtattttaattgctggaagttcgttggctttagcaggcttgataatgcaacaaatgatgcaaaataagtttgttagtccgactacagctggaacgatggaatgggctaaactaggtattttaattgctttattgttctttccaaccggtcatattttattaaaactagtatttgctgttatttgcagtatttgcggtacgtttttatttgttaaaatcattgattttataaaagtgaaagatgtcatttttgtaccgcttttaggaattatgatgggtgggattgttgcaagtttcacaaccttcatctcattgcgcacgaatgctgttcaaagcattggtaactggcttaacgggaactttgccattatcacaagtggacgctatgaaattttatatttaagtattcctcttttagcattgacatatctttttgctaatcatttcacgattgtaggaatgggtaaagactttactaataatttaggtttgagttacgaaaaattaattaacatcgcattgtttattactgcaactattacagcattggtagtggtgactgttggaacattaccgttcttaggactagtaataccaaatattatttcaatttatcgaggtgatcatttgaaaaatgctatccctcatacgatgatgttaggtgccatctttgtattattttctgatatagttggcagaattgttgtttatccatatgaaataaatattggtttaacaataggtgtatttggaacaatcattttccttatcttgcttatgaaaggtaggaaaaattatgcgcaacaataa

SEQ ID NO: 4 - DNA, 909 bp, Staphylococcus aureus
acagcaactttagcagttggtttaatagcccctttagccaatccatttatagaaatttctaaagcagaaaataagatagaagatatcggtcaaggtgcagaaatcatcaaaagaacacaagacattactagcaaacgattagctataactcaaaacattcaatttgattttgtaaaagataaaaaatataacaaagatgccctagttgttaagatgcaaggcttcatcagctctagaacaacatattcagacttaaaaaaatatccatatattaaaagaatgatatggccatttcaatataatatcagtttgaaaacgaaagactctaatgttgatttaatcaattatcttcctaaaaataaaattgattcagcagatgttagtcagaaattaggctataatatcggcggaaacttccaatcagcgccatcaatcggaggcagtggctcattcaactactctaaaacaattagttataatcaaaaaaactatgttactgaagtagaaagtcagaactctaaaggtgttaaatggggagtgaaagcaaattcatttgttacaccgaatggtcaagtatctgcatatgatcaatacttatttgcacaagacccaactggtccagcagcaagagactatttcgtcccagataatcaattacctcctttaattcaaagtggctttaatccatcatttattacaacattgtcacacgaaagaggtaaaggtgataaaagcgagtttgaaatcacttacggcagaaacatggatgctacatatgcttacgtgacaagacatcgtttagccgttgatagaaaacatgatgcttttaaaaaccgaaacgttacagttaaatatgaagtgaactggaaaacacatgaagtaaaaattaaaagcatcacacctaagtaa

SEQ ID NO: 5 - DNA, 1053 bp, Staphylococcus aureus
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 6 - DNA, 1527 bp, Staphylococcus aureus
atgagtagtcatattcaaatttttgatacgacactaagagacggtgaacaaacaccaggagtgaattttacttttgatgaacgcttgcgtattgcattgcaattagaaaaatggggtgtagatgttattgaagctggatttcctgcttcaagtacaggtagctttaaatctgttcaagcaattgcacaaacattaacaacaacggctgtatgtggtttagctagatgtaaaaaatctgacatcgatgctgtatatgaagcaacaaaagatgcagcgaagccggtcgtgcatgtttttatagcaacatcacctattcatcttgaacataaacttaaaatgtctcaagaagacgttttagcatctattaaagaacatgtcacatacgcgaaacaattatttgacgttgttcaattttcacctgaagatgcaacgcgtactgaattaccattcttagtgaaatgtgtacaaactgccgttgacgctggagctacagttattaatattcctgatacagtcggctacagttaccatgatgaatatgcacatattttcaaaaccttaacagaatctgtaacatcttcaaatgaaattatttatagtgctcattgccatgacgatttaggaatggctgtttcaaatagtttagctgcaattgaaggcggtgcgagacgaattgaaggcactgtaaatggtattggtgaacgagcaggtaatgcagcacttgaagaagtcgcgcttgcactatacgttcgaaatgatcattatggtgctcaaactgcccttaatctcgaagaaactaaaaaaacatcggatttaatttcaagatatgcaggtattcgagtgcctagaaataaagcaattgttggccaaaatgcatttagtcatgaatcaggtattcaccaagatggcgtattaaaacatcgtgaaacatatgaaattatgacacctcaacttgttggtgtaagcacgactgaacttccattaggaaaattatctggtaaacacgccttctcagagaagttaaaagcattaggttataacattgataaagaagcgcaaatagatttatttaaacaattcaagaccattgcggacaaaaagaaatctgtttcagatagagatattcatgcgattattcaaggttctgagcatgagcatcaagcactttataaattggaaacactacaactacaatatgtctctagcggccttcaaagtgctgttgttgttgttaaagataaagagggtcatatttaccaggattcaagtattggtactggttcaatcgtagcaatttacaatgcagttgatcgtattttccagaaagaaacagaattaattgattatcgtattaattctgtcactgaaggtactgatgcccaagcagaagtacatgtaaatttattgattgaaggtaagactgtcaatggctttggtattgatcatgatattttacaagcctcttgtaaagcatacgtagaagcacatgctaaatttgcagctgaaaatgttgagaaggtaggtaat

SEQ ID NO: 7 - DNA, 2700 bp, Staphylococcus aureus
atgaaaaaaagaattgattatttgtcgaataagcagaataagtattcgattagacgttttacagtaggtaccacatcagtaatagtaggggcaactatactatttgggataggcaatcatcaagcacaagcttcagaacaatcgaacgatacaacgcaatcttcgaaaaataatgcaagtgcagattccgaaaaaaacaatatgatagaaacacctcaattaaatacaacggctaatgatacatctgatattagtgcaaacacaaacagtgcgaatgtagatagcacaacaaaaccaatgtctacacaaacgagcaataccactacaacagagccagcttcaacaaatgaaacacctcaaccgacggcaattaaaaatcaagcaactgctgcaaaaatgcaagatcaaactgttcctcaagaagcaaattctcaagtagataataaaacaacgaatgatgctaatagcatagcaacaaacagtgagcttaaaaattctcaaacattagatttaccacaatcatcaccacaaacgatttccaatgcgcaaggaactagtaaaccaagtgttagaacgagagctgtacgtagtttagctgttgctgaaccggtagtaaatgctgctgatgctaaaggtacaaatgtaaatgataaagttacggcaagtaatttcaagttagaaaagactacatttgaccctaatcaaagtggtaacacatttatggcggcaaattttacagtgacagataaagtgaaatcaggggattattttacagcgaagttaccagatagtttaactggtaatggagacgtggattattctaattcaaataatacgatgccaattgcagacattaaaagtacgaatggcgatgttgtagctaaagcaacatatgatatcttgactaagacgtatacatttgtctttacagattatgtaaataataaagaaaatattaacggacaattttcattacctttatttacagaccgagcaaaggcacctaaatcaggaacatatgatgcgaatattaatattgcggatgaaatgtttaataataaaattacttataactatagttcgccaattgcaggaattgataaaccaaatggcgcgaacatttcttctcaaattattggtgtagatacagcttcaggtcaaaacacatacaagcaaacagtatttgttaaccctaagcaacgagttttaggtaatacgtgggtgtatattaaaggctaccaagataaaatcgaagaaagtagcggtaaagtaagtgctacagatacaaaactgagaatttttgaagtgaatgatacatctaaattatcagatagctactatgcagatccaaatgactctaaccttaaagaagtaacagaccaatttaaaaatagaatctattatgagcatccaaatgtagctagtattaaatttggtgatattactaaaacatatgtagtattagtagaagggcattacgacaatacaggtaagaacttaaaaactcaggttattcaagaaaatgttgatcctgtaacaaatagagactacagtattttcggttggaataatgagaatgttgtacgttatggtggtggaagtgctgatggtgattcagcagtaaatccgaaagacccaactccagggccgccggttgacccagaaccaagtccagacccagaaccagaaccaacgccagatccagaaccaagtccagacccagaaccggaaccaagcccagacccggatccggattcggattcagacagtgactcaggctcagacagcgactcaggttcagatagcgactcagaatcagatagcgattcggattcagacagtgattcagattcagacagcgactcagaatcagatagcgattcagaatcagatagcgactcagattcagatagcgattcagattcagatagcgattcagaatcagatagcgattcggattcagacagtgattcagattcagacagcgactcagaatcagatagcgactcagaatcagatagtgagtcagattcagacagtgactcggactcagacagtgattcagactcagatagcgattcagactcagatagcgattcagactcagacagcgattcagattcagacagcgactcagaatcagacagcgactcagactcagatagcgactcagactcagacagcgactcagattcagatagcgattcagactcagacagcgactcagactcagacagcgactcagactcagatagcgattcagactcagacagcgactcagattcagatagcgattcggactcagacagcgattcagattcagacagcgactcagactcggatagcgattcagattcagacagcgactcagactcggatagcgactcggattcagatagtgactccgattcaagagttacaccaccaaataatgaacagaaagcaccatcaaatcctaaaggtgaagtaaaccattctaataaggtatcaaaacaacacaaaactgatgctttaccagaaacaggagataagagcgaaaacacaaatgcaactttatttggtgcaatgatggcattattaggatcattactattgtttagaaaacgcaagcaagatcataaagaaaaagcgtaaatacttttttaggccgaatacatttgtattcggtttttttgttgaaaatgattttaaagtgaattg

SEQ ID NO: 8 - DNA, 2673 bp, Staphylococcus aureus
atggctgaattacctcaatcaagaataaatgaacgaaatattaccagtgaaatgcgtgaatcatttttagattatgcgatgagtgttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataaagatacaatagattttatcgataactatgatggtaatgaaagagagccgtcagtcttacctgctcgattccctaacttgttagccaatggagcatcaggtatagcggtaggtatggcaacgaatattccaccacataacttaacagaattaatcaatggtgtacttagcttaagtaagaaccctgatatttcaattgctgagttaatggaggatattgaaggtcctgatttcccaactgctggacttattttaggtaagagtggtattagacgtgcatatgaaacaggtcgtggttcaattcaaatgcgttctcgtgcagttattgaagaacgtggaggcggacgtcaacgtattgttgtcactgaaattcctttccaagtgaataaggctcgtatgattgaaaaaattgcagagctcgttcgtgacaagaaaattgacggtatcactgatttacgtgatgaaacaagtttacgtactggtgtgcgtgtcgttattgatgtgcgtaaggatgcaaatgctagtgtcattttaaataacttatacaaacaaacacctcttcaaacatcatttggtgtgaatatgattgcacttgtaaatggtagaccgaagcttattaatttaaaagaagcgttggtacattatttagagcatcaaaagacagttgttagaagacgtacgcaatacaacttacgtaaagctaaagatcgtgcccacattttagaaggattacgtatcgcacttgaccatatcgatgaaattatttcaacgattcgtgagtcagatacagataaagttgcaatggaaagcttgcaacaacgcttcaaactttctgaaaaacaagctcaagctattttagacatgcgtttaagacgtctaacaggtttagagagagacaaaattgaagctgaatataatgagttattaaattatattagtgaattagaaacaatcttagctgatgaagaagtattactacaattagttagagatgaattaacagaaattcgagatcgtttcggtgatgatcgtcgtactgaaatccaattaggtggatttgaagatttagaagatgaagatctcattccagaagaacaaattgtaattacactaagccataataactacattaaacgtttgccggtatctacatatcgtgctcaaaaccgtggtggtcgtggtgttcaaggtatgaatacattggaagaagattttgtcagtcaattggtaactttaagtacacatgaccatgtattgttctttactaacaaaggtcgtgtatacaaacttaaaggttatgaagtgcctgagttatcaagacagtctaaaggtattcctgtagtgaatgctattgaacttgaaaatgatgaagtcattagtacaatgattgctgttaaagaccttgaaagtgaagacaacttcttagtgtttgcaactaaacgtggtgtcgttaaacgttcagcattaagtaacttctcaagaataaatagaaatggtaagattgcgatttcgttcagagaagatgatgagttaattgcagttcgcttaacaagtggtcaagaagatatcttgattggtacatcacatgcatcattaattcgattccctgaatcaacattacgtcctttaggccgtacagcaacgggtgtgaaaggtattacacttcgtgaaggtgacgaagttgtagggcttgatgtagctcatgcaaacagtgttgatgaagtattagtagttactgaaaatggttatggtaaacgtacgccagttaatgactatcgtttatcaaatcgtggtggtaaaggtattaaaacagctacgattactgagcgtaatggtaatgttgtatgtatcactacagtaactggtgaagaagatttaatgattgttactaatgcaggtgtcattattcgactagatgttgcagatatttctcaaaatggtcgtgcagcacaaggtgttcgcttaattcgcttaggtgatgatcaatttgtttcaacggttgctaaagtaaaagaagatgcagaagatgaaacgaatgaagatgagcaatctacttcaactgtatctgaagatggtactgaacaacaacgtgaagcggttgtaaatgatgaaacaccaggaaatgcaattcatactgaagtgattgattcagaagaaaatgatgaagatggacgtattgaagtaagacaagatttcatggatcgtgttgaagaagatatacaacaatcatcagatgaagatgaagaataataa

SEQ ID NO: 9 - DNA, 29 bp, Artificial Sequence
  Primer; TKO1; leuA PCR Amplification with Sph1; upstream pr
gatgcgcatgcgaaacagattatctattc

SEQ ID NO: 10 - DNA, 29 bp, Artificial Sequence
  Primer; TKO2; LeuA PCR Amplification with Sph1 (upstream pr-alternate)
gatgcgcatgccagattatctattcaaag

SEQ ID NO: 11 - DNA, 32 bp, Artificial Sequence
  Primer; TKO3; LeuA PCR Amplification with Pst1 (downstream pr)
catgatctgcagagtaaattcccccgtaaatt

SEQ ID NO: 12 - DNA, 32 bp, Artificial Sequence
  Primer; TKO4; LeuA PCR Amplification with Pst1 (downstream pr-alternate)
cacgtgatctgcagagtaaattcccccgtaaa

SEQ ID NO: 13 - DNA, 32 bp, Artificial Sequence
  Primer; TKO5; upstream primer to amplify ClfB promoter with EcoRI
gactacgaattcaggtgatgaaaaatttagaa

SEQ ID NO: 14 - DNA, 32 bp, Artificial Sequence
  Primer; TKO6; backup upstream primer to amplify ClfB promoter with EcoRI
gactacgaattctgatgaaaaatttagaactt

SEQ ID NO: 15 - DNA, 32 bp, Artificial Sequence
  Primer; TKO7; downstream primer to amplify ClfB promoter with BamHI
cttagctggatccaaatattactccatttcaa

SEQ ID NO: 16 - DNA, 36 bp, Artificial Sequence
  Primer; TKO8; backup downstream primer to amplify ClfB promoter with BamHI
cttagctggatccaaatattactccatttcaatttc

SEQ ID NO: 17 - DNA, 31 bp, Artificial Sequence
  Primer; TKO9; upstream primer to amplify the hlgA RR; contains Sph1
gatgcgcatgctcacaaactattgcgaaatc

SEQ ID NO: 18 - DNA, 32 bp, Artificial Sequence
  Primer; TKO10; backup upstream primer to amplify the hlgA RR
gatgcgcatgcaaactattgcgaaatccattc

SEQ ID NO: 19 - DNA, 32 bp, Artificial Sequence
  Primer; TKO11; downstream primer to amplify hlgA RR; contains pstI
catgatctgcagatatataataatccatttgt

SEQ ID NO: 20 - DNA, 37 bp, Artificial Sequence
  Primer; TKO12; backup downstream primer to amplify hlgA RR
catgatctgcagatatataataatccatttgtaagcg

SEQ ID NO: 21 - DNA, 20 bp, Artificial Sequence
  Primer; TKO13; First sense primer for sequencing constructs containing pCAD promoter
gtgttacgatagcaaatgca

SEQ ID NO: 22 - DNA, 20 bp, Artificial Sequence
  Primer; TKO14; second sense sequencing primer anneals roughly in the middle of the SprA1 gene
ttattggctaagtagacgca

SEQ ID NO: 23 - DNA, 20 bp, Artificial Sequence
  Primer; TKO15; primer to anneal just upstream of the serum responsive RRs for leuA and hlgA. Anneals in the PCN51 vector about 75 nt upstream of the Sph1 site
cacatgttctttcctgcgtt

SEQ ID NO: 24 - DNA, 20 bp, Artificial Sequence
  Primer; TKO16; backup primer to anneal just upstream of the serum responsive RRs for leuA and hlgA
acgcggcctttttacggttc

SEQ ID NO: 25 - DNA, 20 bp, Artificial Sequence
  Primer; TKO17; primer to anneal near the downstream one third of the leuA promoter/RR
gaatgggacttgtaaacgtc

SEQ ID NO: 26 - DNA, 18 bp, Artificial Sequence
  Primer; TKO18; backup primer to anneal near the downstream one third of the leuA promoter/RR
gaatgggacttgtaaacg

SEQ ID NO: 27 - DNA, 20 bp, Artificial Sequence
  Primer; TKO19; primer to anneal near the downstream one third of the hlgA promoter/RR
ataaacgcctgcgaccaata

SEQ ID NO: 28 - DNA, 20 bp, Artificial Sequence
  Primer; TKO20; backup primer to anneal near the downstream one third of the hlgA promoter/RR
gcgaccaataaatcttttaa

SEQ ID NO: 29 - DNA, 40 bp, Artificial Sequence
  Primer; TKO21; pTK1 vector with leuA pro homology R
ttgaatagataatctgtttcgcatgcagcggccgccagct

SEQ ID NO: 30 - DNA, 40 bp, Artificial Sequence
  Primer; TKO22; pTK1 vector with leuA pro homology F
aatttacgggggaatttactctgcagggtaccgcagagag

SEQ ID NO: 31 - DNA, 49 bp, Artificial Sequence
  Primer; TKO23; leuA insert with pTK1 homology F
agctggcggccgctgcatgcgaaacagattatctattcaaagttaattg

SEQ ID NO: 32 - DNA, 46 bp, Artificial Sequence
  Primer; TKO24; leuA insert with pTK1 homology R
ctctctgcggtaccctgcagagtaaattcccccgtaaattttaatg

SEQ ID NO: 33 - DNA, 46 bp, Artificial Sequence
  Primer; TKO25; pTK9 vector with leuA pro homology F
cattaaaatttacgggggaatttactctgcagatgagcagggatga

SEQ ID NO: 34 - DNA, 49 bp, Artificial Sequence
  Primer; TKO26; pTK9 vector with leuA pro homology R
caattaactttgaatagataatctgtttcgcatgcagcggccgccagct

SEQ ID NO: 35 - DNA, 46 bp, Artificial Sequence
  Primer; TKO27; pTK12 vector with leuA pro homology F
cattaaaatttacgggggaatttactctgcagatggtagagatagc

SEQ ID NO: 36 - DNA, 46 bp, Artificial Sequence
  Primer; TKO28; leuA insert with pTK12 homology R
gctatctctaccatctgcagagtaaattcccccgtaaattttaatg

SEQ ID NO: 37 - DNA, 49 bp, Artificial Sequence
  Primer; TKO29; pTKvector R with kanR homology
gcaatccatcttgttcaatcattataaccctctttaatttggttatatg

SEQ ID NO: 38 - DNA, 44 bp, Artificial Sequence
  Primer; TKO30; pTKvector F with kanR homology
ccttcttgacgagttcttctgagttaagggatgcataaactgca

SEQ ID NO: 39 - DNA, 49 bp, Artificial Sequence
  Primer; TKO31; pCASSA kanR F with pTK homology
catataaccaaattaaagagggttataatgattgaacaagatggattgc

SEQ ID NO: 40 - DNA, 44 bp, Artificial Sequence
  Primer; TKO32; pCASSA kanR R with pTK homology
tgcagtttatgcatcccttaactcagaagaactcgtcaagaagg

SEQ ID NO: 41 - DNA, 22 bp, Artificial Sequence
  Primer; TKO33; leuA colony screen PCR F
gaatgggacttgtaaacgtccc

SEQ ID NO: 42 - DNA, 22 bp, Artificial Sequence
  Primer; TKO34; leuA colony screen PCR R
gggacgtttacaagtcccattc

SEQ ID NO: 43 - DNA, 24 bp, Artificial Sequence
  Primer; BPC - T1.1 - FOR; gRNA insertion for pCasSA
gaaaggagtaatatcgatggagta

SEQ ID NO: 44 - DNA, 24 bp, Artificial Sequence
  Primer; BPC - T1.1 - REV; gRNA insertion for pCasSA
caaatactccatcgatattactcc

SEQ ID NO: 45 - DNA, 24 bp, Artificial Sequence
  Primer; gRNA insertion for pCasSA
gaaaggagaggatgatgattataa

SEQ ID NO: 46 - DNA, 24 bp, Artificial Sequence
  Primer; BPC - T1.2 - REV; gRNA insertion for pCasSA
caaattataatcatcatcctctcc

SEQ ID NO: 47 - DNA, 24 bp, Artificial Sequence
  Primer; BPC - T1.3 - FOR; gRNA insertion for pCasSA
gaaagggagaggatgatgattata

SEQ ID NO: 48 - DNA, 24 bp, Artificial Sequence
  Primer; BPC - T1.3 - REV; gRNA insertion for pCasSA
caaatataatcatcatcctctccc

SEQ ID NO: 49 - DNA, 24 bp, Artificial Sequence
  Primer; BPC - T1.4 - FOR; gRNA insertion for pCasSA
gaaagggtctaatgttattgctta

SEQ ID NO: 50 - DNA, 24 bp, Artificial Sequence
  Primer; BPC - T1.4 - REV; gRNA insertion for pCasSA
caaataagcaataacattagaccc

SEQ ID NO: 51 - DNA, 23 bp, Artificial Sequence
  Primer; BPC - T1.5 - FOR; gRNA insertion for pCasSA
gaaaggagaggatgatgattata

SEQ ID NO: 52 - DNA, 23 bp, Artificial Sequence
  Primer; BPC - T1.5 - REV; gRNA insertion for pCasSA
caaatataatcatcatcctctcc

SEQ ID NO: 53 - DNA, 24 bp, Artificial Sequence
  Primer; BPC - T1.6 - FOR; gRNA insertion for pCasSA
gaaaggtagtatgagtaatatcga

SEQ ID NO: 54 - DNA, 24 bp, Artificial Sequence
  Primer; BPC - T1.6 - REV; gRNA insertion for pCasSA
caaatcgatattactcatactacc

SEQ ID NO: 55 - DNA, 24 bp, Artificial Sequence
  Primer; BPC - T1.7 - FOR; gRNA insertion for pCasSA
gaaaggaattatataaatataaag

SEQ ID NO: 56 - DNA, 24 bp, Artificial Sequence
  Primer; BPC - T1.7 - REV; gRNA insertion for pCasSA
caaactttatatttatataattcc

SEQ ID NO: 57 - DNA, 24 bp, Artificial Sequence
  Primer; BPC - T1.8 - FOR; gRNA insertion for pCasSA
gaaaggctacctccatattttcta

SEQ ID NO: 58 - DNA, 24 bp, Artificial Sequence
  Primer; BPC - T1.8 - REV; gRNA insertion for pCasSA
caaatagaaaatatggaggtagcc

SEQ ID NO: 59 - DNA, 24 bp, Artificial Sequence
  Primer; BPC - T1.9 - FOR; gRNA insertion for pCasSA
gaaaggatagaactgtattagact

SEQ ID NO: 60 - DNA, 24 bp, Artificial Sequence
  Primer; BPC - T1.9 - REV; gRNA insertion for pCasSA
caaaagtctaatacagttctatcc

SEQ ID NO: 61 - DNA, 24 bp, Artificial Sequence
  Primer BPC - T1.10 - FOR; gRNA insertion for pCasSA
gaaaggtgtctaatgttattgctt

SEQ ID NO: 62 - DNA, 24 bp, Artificial Sequence
  Primer; BPC - T1.10 - REV; gRNA insertion for pCasSA
caaaaagcaataacattagacacc

SEQ ID NO: 63 - DNA, 20 bp, Artificial Sequence
  Primer; BPC - gRNA - FOR; sequencing primer for gRNA insertion into the pCasSA vector
tgttctttcctgcgttgtcg

SEQ ID NO: 64 - DNA, 24 bp, Artificial Sequence
  Primer; BPC - gRNA - REV; sequencing primer for gRNA insertion into the pCasSA vector
tcgcattgacgttaatacctacat

SEQ ID NO: 65 - DNA, 24 bp, Artificial Sequence
  Primer; BPC - T1.1.2 - REV; reverse primer for gRNA
aaactactccatcgatattactcc

SEQ ID NO: 66 - DNA, 24 bp, Artificial Sequence
  Primer; BPC - T1.2.2 - REV; reverse primer for gRNA
aaacttataatcatcatcctctcc

SEQ ID NO: 67 - DNA, 24 bp, Artificial Sequence
  Primer BPC - T1.3.2 - REV; reverse primer for gRNA
aaactataatcatcatcctctccc

SEQ ID NO: 68 - DNA, 24 bp, Artificial Sequence
  Primer; BPC - T1.4.2 - REV; reverse primer for gRNA
aaactaagcaataacattagaccc

SEQ ID NO: 69 - DNA, 23 bp, Artificial Sequence
  Primer; BPC - T1.5.2 - REV; reverse primer for gRNA
aaactataatcatcatcctctcc

SEQ ID NO: 70 - DNA, 24 bp, Artificial Sequence
  Primer; BPC - T1.6.2 - REV; reverse primer for gRNA
aaactcgatattactcatactacc

SEQ ID NO: 71 - DNA, 24 bp, Artificial Sequence
  Primer; BPC - T1.7.2 - REV; reverse primer for gRNA
aaacctttatatttatataattcc

SEQ ID NO: 72 - DNA, 24 bp, Artificial Sequence
  Primer; BPC - T1.8.2 - REV; reverse primer for gRNA
aaactagaaaatatggaggtagcc

SEQ ID NO: 73 - DNA, 24 bp, Artificial Sequence
  Primer; BPC - T1.9.2 - REV; reverse primer for gRNA
aaacagtctaatacagttctatcc

SEQ ID NO: 74 - DNA, 24 bp, Artificial Sequence
  Primer; BPC - T1.10.2 - REV; reverse primer for gRNA
aaacaagcaataacattagacacc

SEQ ID NO: 75 - DNA, 20 bp, Artificial Sequence
  Primer; BPC - pCN51-1 - FOR; Primer to check insertion into pCN51
tttgctggccttttgctcac

SEQ ID NO: 76 - DNA, 24 bp, Artificial Sequence
  Primer; BPC - pCN51-1 - REV; Primer to check insertion into pCN51
tgctttttcgattgatgaacacct

SEQ ID NO: 77 - DNA, 20 bp, Artificial Sequence
  Primer; BPC - pCN51-2 - FOR; Primer to check insertion into pCN51
cggcctttttacggttcctg

SEQ ID NO: 78 - DNA, 24 bp, Artificial Sequence
  Primer; BPC - pCN51-2 - REV; Primer to check insertion into pCN51
acgttgctttttcgattgatgaac

SEQ ID NO: 79 - DNA, 33 bp, Artificial Sequence
  Primer; BPC - mChr-1 - FOR; mCherry mRNA with Pst1
cacgtgatctgcagtcacatggtgagcaagggc

SEQ ID NO: 80 - DNA, 33 bp, Artificial Sequence
  Primer; BPC - mChr-1 - REV; mCherry mRNA with EcoR1
gactacgaattcaaaactgatttcgttgacccg

SEQ ID NO: 81 - DNA, 34 bp, Artificial Sequence
  Primer; BPC - mChr-2 - REV; mCherry mRNA with BamH1
cttagctggatccaaaactgatttcgttgacccg

SEQ ID NO: 82 - DNA, 37 bp, Artificial Sequence
  Primer; BPC - pCN51-hdr - REV; 34BPC-REV with Xma1, use with TKO15 to add homologous arms
cttagctcccgggtgctttttcgattgatgaacacct

SEQ ID NO: 83 - DNA, 20 bp, Artificial Sequence
  Primer; BPC - 2a - FOR; 502a Target 1 genomic incorp check
cgccaaacgtttcgtcagtt

SEQ ID NO: 84 - DNA, 20 bp, Artificial Sequence
  Primer; BPC - 2a - REV; 502a Target 1 genomic incorp check
ttcaagcgtgacaaagcagc

SEQ ID NO: 85 - DNA, 20 bp, Artificial Sequence
  Primer; BPC - 2a - FOR; 502a Target 1 genomic incorp check
tgcgcaatggccaaaaagat

SEQ ID NO: 86 - DNA, 20 bp, Artificial Sequence
  Primer; BPC - 2a - REV; 502a Target 1 genomic incorp check
cgtgctaacatccgcttcaa

SEQ ID NO: 87 - DNA, 21 bp, Artificial Sequence
  Primer; BPC - mChr-1 - REV; mCherry
aaaactgatttcgttgacccg

SEQ ID NO: 88 - DNA, 33 bp, Artificial Sequence
  Primer; BPC - mChr-1 - FOR; mCherry with Pst1
cacgtgatctgcagtcacatggtttctaaaggt

SEQ ID NO: 89 - DNA, 22 bp, Artificial Sequence
  Primer; BPC - pJ204-1 - FOR; Checking for insertion btwn HAs in pJ204
acgttgctttttcgattgatga

SEQ ID NO: 90 - DNA, 19 bp, Artificial Sequence
  Primer; Checking for insertion btwn HAs in pJ204; Checking for insertion btwn HAs in pJ204
tccccatgcgagagtaggg

SEQ ID NO: 91 - DNA, 23 bp, Artificial Sequence
  Primer; BPC - pJ204-2 - FOR; Checking for insertion btwn HAs in pJ204
gaatatttaagggcgcctgtcac

SEQ ID NO: 92 - DNA, 18 bp, Artificial Sequence
  Primer; BPC - pJ204-2 - REV; Checking for insertion btwn HAs in pJ204
tatggggtgtcgcccttt

SEQ ID NO: 93 - DNA, 19 bp, Artificial Sequence
  Primer; BPC - mChr-1 - FOR; mCherry codon opt seq in pJ204 300995
tcacatggtttctaaaggt

SEQ ID NO: 94 - DNA, 20 bp, Artificial Sequence
  Primer; BP - repF-1 - F; Checking for repF removal
catgcctgcagaacggattg

SEQ ID NO: 95 - DNA, 20 bp, Artificial Sequence
  Primer; BP - repF-1 - R; Checking for repF removal
gcgcgggaatatgatgctaa

SEQ ID NO: 96 - DNA, 20 bp, Artificial Sequence
  Primer; BP - repF-2 - F; Checking for repF removal
aggtgactgatggctggttg

SEQ ID NO: 97 - DNA, 20 bp, Artificial Sequence
  Primer; BP - repF-2 - R; Checking for repF removal
tatgtcttttgcgcagtcgg

SEQ ID NO: 98 - DNA, 24 bp, Artificial Sequence
  Primer; BP - srtA - F; srtA CRISPR targeting from Dong et al.
gaaacaaacaaatatgctgccact

SEQ ID NO: 99 - DNA, 24 bp, Artificial Sequence
  Primer; BP - srtA - R; srtA CRISPR targeting
aaacagtggcagcatatttgtttg

SEQ ID NO: 100 - DNA, 24 bp, Artificial Sequence
  Primer; BP - hla - F; hla CRISPR targeting from Dong et al.
gaaagcttccaatatctgtagtac

SEQ ID NO: 101 - DNA, 24 bp, Artificial Sequence
  Primer; BP - hla - R; BP - hla - R
aaacgtactacagatattggaagc

SEQ ID NO: 102 - DNA, 24 bp, Artificial Sequence
  Primer; BP - coa - F; coa CRISPR targeting from Dong et al.
gaaagccatttttaaatctgtacg

SEQ ID NO: 103 - DNA, 24 bp, Artificial Sequence
  Primer; BP - coa - R; coa CRISPR targeting
aaaccgtacagatttaaaaatggc

SEQ ID NO: 104 - protein, 30 aa, Staphylococcus aureus
MLIFVHIIAPVISGCAIAFFSYWLSRRNTK

SEQ ID NO: 105 - protein, 31 aa, Artificial Sequence
  pepA1- related antimicrobial peptide; WO 2013/050590
MMLIFVHIIAPVISGCAIAFFSYWLSRRNTK

SEQ ID NO: 106 - protein, 15 aa, Artificial Sequence
  pepA1- related antimicrobial peptide; WO 2013/050590
AIAFFSYWLSRRNTK

SEQ ID NO: 107 - protein, 14 aa, Artificial Sequence
  pepA1- related antimicrobial peptide; WO 2013/050590
IAFFSYWLSRRNTK

SEQ ID NO: 108 - protein, 13 aa, Artificial Sequence
  pepA1- related antimicrobial peptide; WO 2013/050590
AFFSYWLSRRNTK

SEQ ID NO: 109 - protein, 12 aa, Artificial Sequence
  pepA1- related antimicrobial peptide; WO 2013/050590
FFSYWLSRRNTK

SEQ ID NO: 110 - protein, 11 aa, Artificial Sequence
  pepA1- related antimicrobial peptide; WO 2013/050590
FSYWLSRRNTK

SEQ ID NO: 111 - protein, 10 aa, Artificial Sequence
  pepA1- related antimicrobial peptide; WO 2013/050590
SYWLSRRNTK

SEQ ID NO: 112 - protein, 9 aa, Artificial Sequence
  pepA1- related antimicrobial peptide; WO 2013/050590
YWLSRRNTK

SEQ ID NO: 113 - protein, 33 aa, Staphylococcus aureus
MQGFKEKHQELKKALCQIGLMRSISEVKQLNIA

SEQ ID NO: 114 - DNA, 402 bp, Staphylococcus aureus
gcatgcgaaacagattatctattcaaagttaattgtaagaaaatttaaaatatttgttgacatactaaagcagatatagtaaattaaatttatcaaatttttagacaattctaactattaaagtgatatataccattcacggaaggagtataataaaatgcttaatcaatatactgaacatcaaccgacaacttcaaatattattattttattatactctttaggactcgaacgttagtaaatatttactaaacgctttaagtcctatttctgtttgaatgggacttgtaaacgtcccaataatattgggacgtttttttatgttttatctttcaattacttatttttattactataaaacatgattaatcattaaaatttacgggggaatttactctgcag

SEQ ID NO: 115 - DNA, 297 bp, Staphylococcus aureus
gcatgcaaactattgcgaaatccattcctcttccactacaagcaccataattaaacaacaattcaatagaataagacttgcaaaacatagttatgtcgctatataaacgcctgcgaccaataaatcttttaaacataacataatgcaaaaacatcatttaacaatgctaaaaatgtctcttcaatacatgttgatagtaattaacttttaacgaacagttaattcgaaaacgcttacaaatggattattatatatatgaacttaaaattaaatagaaagaaagtgatttctctgcag

SEQ ID NO: 116 - DNA, 598 bp, Artificial Sequence
  Promoter; Cadmium promoter sequence between restriction sites SphI and PstI
gcatgcgcacttattcaagtgtattttttaataaattattttacttattgaaatgtattattttctaatgtcataccctggtcaaaaccgttcgtttttgagactagaattttatgccctacttacttcttttattttcattcaaatatttgcttgcatgatgagtcgaaaatggttataatacactcaaataaatatttgaatgaagatgggatgataatatgaaaaagaaagatacttgtgaaattttttgttatgacgaagaaaaggttaatcgaatacaaggggatttacaaacagttgatatttctggtgttagccaaattttaaaggctattgccgatgaaaatagagcaaaaattacttacgctctgtgtcaggatgaagagttgtgtgtttgtgatatagcaaatatcttaggtgttacgatagcaaatgcatctcatcatttacgtacgctttataagcaaggggtggtcaactttagaaaagaaggaaaactagctttatattctttaggtgatgaacatatcaggcagataatgatgatcgccctagcacataagaaagaagtgaaggtcaatgtctgaacctgcag

SEQ ID NO: 117 - DNA, 231 bp, Artificial Sequence
  Promoter; clfB promoter forward sequence with EcoRI and BamHI sites
gaattcaggtgatgaaaaatttagaacttctaagtttttgaaaagtaaaaaatttgtaatagtgtaaaaatagtatattgatttttgctagttaacagaaaattttaagttatataaataggaagaaaacaaattttacgtaatttttttcgaaaagcaattgatataattcttatttcattatacaatttagactaatctagaaattgaaatggagtaatatttggatcc

SEQ ID NO: 118 - DNA, 231 bp, Artificial Sequence
  Promoter; clfB promoter as it is cloned in pCN51 vector with EcoRI and BamHI reversed
ggatccaaatattactccatttcaatttctagattagtctaaattgtataatgaaataagaattatatcaattgcttttcgaaaaaaattacgtaaaatttgttttcttcctatttatataacttaaaattttctgttaactagcaaaaatcaatatactatttttacactattacaaattttttacttttcaaaaacttagaagttctaaatttttcatcacctgaattc

SEQ ID NO: 119 - DNA, 303 bp, Staphylococcus aureus
ttagaaagatttacttttatatatgaagagactggattaaatacttttattgacgtaaaaattcacttttgaaccgttcaatatcttgccgatttttatataacagctacaaataaaatataacagtttgattttacagcctcggtaaatcgtcttgacaaacaaaaattttgtgctatcacaacatttgcaacgtcttaacaagtcatctataaacatttctaaatatttaacattacttatgcgtcatttattgctaaaattattgtattaaaatatacatagaattgatgggatatcatg

SEQ ID NO: 120 - DNA, 303 bp, Staphylococcus aureus
acgaaaaattaattaacatcgcattgtttattactgcaactattacagcattggtagtggtgactgttggaacattaccgttcttaggactagtaataccaaatattatttcaatttatcgaggtgatcatttgaaaaatgctatccctcatacgatgatgttaggtgccatctttgtattattttctgatatagttggcagaattgttgtttatccatatgaaataaatattggtttaacaataggtgtatttggaacaatcattttccttatcttgcttatgaaaggtaggaaaaattatg

SEQ ID NO: 121 - DNA, 303 bp, Staphylococcus aureus
ctatctgcggcatttgcagaattactgaatgtcgcgatgatgataattaacgctaaaatcgttgtattaaaaacttttaaaatatttttcaaaacataatcctcctttttatgattgcttttaagtctttagtaaaatcataaataataatgattatcattgtcaatatttattttataatcaatttattattgttatacggaaatagatgtgctagtataattgataaccattatcaattgcaatggttaatcatctcatataacaacacataatttgtatccttaggaggaaaacaacatg

SEQ ID NO: 122 - DNA, 233 bp, Artificial Sequence
  ORF for sprA1 for plasmid construction
ctgcagggtaccgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttgtggatccttgactgaattc

SEQ ID NO: 123 - DNA, 233 bp, Artificial Sequence
  DNA sequence for the regulatory RNA sprA1sprA1AS (sprA1sprA1 antisense) under the ClfB promoter which is cloned in reverse behind the sprA1 gene, including the antisense regulatory RNA
gaattcagtcaaggatccacaacgaaaataagtatttacttatacaccaatcccctcactatttgcggtagtgaggggatttttattggtgcggctatatgtcacctattttgtattgcgtctacttagccaataagaaaaaaacgcaatggcacagccactgatgactggtgctatgatgtgaacgaaaataagcatcaccttatacacctcctctctgcggtaccctgcag

SEQ ID NO: 124 - DNA, 756 bp, Artificial Sequence
  SmaI DNA sequence between restriction sites PstI and EcoRI
ctgcagatgagcagggatgaccaactctttacactttggggaaagcttaacgatcgtcagaaggataattttctaaaatggatgaaagcttttgatgtagagaaaacttaccaaaaaacaagtggggatattttcaatgatgattttttcgatatatttggtgatagattaattactcatcatttcagtagcacgcaagctttaacaaaaactttattcgaacatgcttttaatgactccttaaatgaatctggagttatatcctctcttgcggaaagtagaacaaaccctgggcatgacataacaatcgatagcataaaggttgctttaaaaacagaagcagctaaaaatattagcaaatcatatattcatgtaagtaagtggatggagttaggcaagggggagtggattctagaattattattagaacggtttttagagcatctagagaattatgaacgtattttcacactcagatattttaaaatatccgagtataaatttagctaccagcttgtagaaatacccaagagtcttttgttggaagcaaaaaatgcgaaattagaaataatgtcgggaagcaaacaaagccctaagcccggctatggatatgtgttagatgaaaatgaaaataagaagttttctctatactttgatggtggtgccgagagaaaacttcaaataaaacatttaaatttagaacattgcattgttcatggagtttgggattttattctaccgccgccttaagaattc

SEQ ID NO: 125 - DNA, 142 bp, Artificial Sequence
  rsaE DNA sequence between restriction sites PstI and EcoRI
ctgcagatggtagagatagcatgttatattatgaacatgaaattaatcacataacaaacatacccctttgtttgaagtgaaaaatttctcccatcccctttgtttagcgtcgtgtattcagacacgacgtttttttgaattc

SEQ ID NO: 126 - DNA, 104 bp, Artificial Sequence
  Variant can be used for RsaE sRNA which may express the sRNA
gaaattaatcacataacaaacatacccctttgtttgaagtgaaaaatttctcccatcccctttgtttagcgtcgtgtattcagacacgacgtttttttgaattc

SEQ ID NO: 127 - DNA, 156 bp, Escherichia coli
atgaagcagcaaaaggcgatgttaatcgccctgatcgtcatctgtttaaccgtcatagtgacggcactggtaacgaggaaagacctctgcgaggtacgaatccgaaccggccagacggaggtcgctgtcttcacagcttacgaacctgaggagtaa

SEQ ID NO: 128 - DNA, 657 bp, Klebsiella pneumoniae
atggatgtctttgataaagtttatagtgatgataataatagttatgaccaaaaaactgtaagtcagcgtattgaagccctatttcttaataaccttggcaaagttgtaactcgtcagcaaatcattagggcggcaactgatccaaaaacagggaaacaaccagaaaattggcatcagagactttcagaactacgaactgataaaggatatactattttatcctggcgggatatgaaggttttagctccgcaagagtatataatgccacacgcaacaagacgcccaaaggcagcaaagcgtgtattaccgacaaaagaaacctgggaacaggttttggatagagctaattactcttgcgagtggcaggaagatggtcaacactgtgggttagttgaaggtgatattgatcctatagggggaggcacggtcaaactaacaccagaccatatgacacctcattcaatagatcccgcaactgatgtaaatgatcctaaaatgtggcaagcattgtgtggacgtcatcaagttatgaaaaaaaattattgggattcaaataatgggaaaataaatgtcattggtatattgcagtcagtaaatgagaaacaaaagaatgatgctttagagtttcttttgaattattatggattgaaaagataa

SEQ ID NO: 129 - DNA, 219 bp, Artificial Sequence
  Promoter; clfB promoter F downregulated in serum
aggtgatgaaaaatttagaacttctaagtttttgaaaagtaaaaaatttgtaatagtgtaaaaatagtatattgatttttgctagttaacagaaaattttaagttatataaataggaagaaaacaaattttacgtaatttttttcgaaaagcaattgatataattcttatttcattatacaatttagactaatctagaaattgaaatggagtaatattt

SEQ ID NO: 130 - DNA, 219 bp, Artificial Sequence
  Promoter; clfB promoter R downregulated in serum
tccactactttttaaatcttgaagattcaaaaacttttcattttttaaacattatcacatttttatcatataactaaaaacgatcaattgtcttttaaaattcaatatatttatccttcttttgtttaaaatgcattaaaaaaagcttttcgttaactatattaagaataaagtaatatgttaaatctgattagatctttaactttacctcattataaa

SEQ ID NO: 131 - DNA, 5743 bp, Artificial Sequence
  Plasmid; pIMAY Integrative Plasmid accession number JQ62198
gcatgcgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaataatagtaatattatacaaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacattctataatagaaggtatggaggatgttatataatgagacagaattatgatgatcatatgtcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatgggtttcactctccttctacattttttaacctaataatgccaaataccgtttgccacccctctctttgataattataatattggcgaaattcgcttctaaagatgaaacgcaatattatatgcttgctttatcggccgtatgtgattataccagccccctcactacatgtcaagaataaactgccaaagcataatgggataattaaccctcactaaagggaacaaaagctgggtaccgggccccccctcgaggtcgacggtatcgataagcttgatatcgaattcctgcagcccgggggatccactagttctagagcggccgccaccgcggtggagctccaattcgccctatagtgagtcgtattacgacgtcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctccgccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagaggagatcttgatctaatgattcaaacccttgtgaacttctttagaacaaaagaggttcgtaacaagattttcttcacactagcaatgttagtaatttttaaaatagggacttatataccagctccaggagtaaatcctgcagcttttgataatccccaaggttctcaaggtgccactgagttattaaatacttttggtggcggagccttgaaacgattttctatttttgcaatgggtattgtaccctacatcactgcatcaatcgtaatgcaattattacaaatggatattgtccctaaattctcagaatgggcaaaacaaggtgaagtaggtagaagaaagttaaataacgttactcgttatttagcaatttctttagcatttatccaatctataggtatggcattccaatttaataattatctcaaaggtgcgctgattatcaatcagtcaattatgagttatttattaatagcactagttttgacagcaggaactgctttcttaatatggcttggtgatcaaatcactcagttcggtgttggtaatggtatttctattatcatattcccatcaagcttattttaattatactctatcaatgatagagtgtcaatattttttttagtttttcatgaactcgaggggatccaaataaaaaactagtttgacaaataactctatcaatgatagagtgtcaacaaaaaggaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttca

SEQ ID NO: 132 - DNA, 303 bp, Artificial Sequence
  Promoter; promoter leuA
atttttagacaattctaactattaaagtgatatataccattcacggaaggagtataataaaatgcttaatcaatatactgaacatcaaccgacaacttcaaatattattattttattatactctttaggactcgaacgttagtaaatatttactaaacgctttaagtcctatttctgtttgaatgggacttgtaaacgtcccaataatattgggacgtttttttatgttttatctttcaattacttatttttattactataaaacatgattaatcattaaaatttacgggggaatttactatg

SEQ ID NO: 133 - DNA, 303 bp, Artificial Sequence
  Promoter; promoter hlgA2
acttcaaattttcacaaactattgcgaaatccattcctcttccactacaagcaccataattaaacaacaattcaatagaataagacttgcaaaacatagttatgtcgctatataaacgcctgcgaccaataaatcttttaaacataacataatgcaaaaacatcatttaacaatgctaaaaatgtctcttcaatacatgttgatagtaattaacttttaacgaacagttaattcgaaaacgcttacaaatggattattatatatatgaacttaaaattaaatagaaagaaagtgatttctatg

SEQ ID NO: 134 - DNA, 71 bp, Artificial Sequence
  Promoter; promoter hrtAB
gttcatattgagttcatatttcaaccttatactgacgctaaagaagaaatagggagaagtgaatcgatatg

SEQ ID NO: 135 - DNA, 80 bp, Artificial Sequence
  Promoter; promoter hlb
ttcaggctatcaataatgctttgaaatcagcctgtagagtcaataatataccaattattacatcgcacgcattaagacac

SEQ ID NO: 136 - DNA, 80 bp, Artificial Sequence
  Promoter; promoter sbnC
actcattgttcttatttactagcaaaaggtgtatctatacattacatttctaaaagattaggtcataaaaatatagcaat

SEQ ID NO: 137 - DNA, 80 bp, Artificial Sequence
  Promoter; promoter isdI
aactacatccgtgtattcgcatttgttagaagaaaaatttaatgaagaggacaaaaaaacaactaaaattttagaaagta

SEQ ID NO: 138 - DNA, 64 bp, Artificial Sequence
  Promoter; promoter isdG
tgtaatttagggacccattagggactccaaacccaataaatactgttgttacaaggtttctatg

SEQ ID NO: 139 - DNA, 303 bp, Artificial Sequence
  Promoter; promoter sbnE
gaatacttcaaggattaacatatagtgcattgattcaaagtgtcatgtttgttgtcgtgaatgcgtgtcatcaacaacttaaaggcacatttgttggaacgacgaacagtatgttagttgttggtcaaattattggcagtcttagtggcgctgccattacaagttatactacaccagctactacgtttatcgttatgggcgtagtatttgcagtaagtagtttatttttaatttgttcaaccatcactaatcaaatcaacgatcacacattaatgaaattatgggagttgaaacaaaaaagtg

SEQ ID NO: 140 - DNA, 303 bp, Artificial Sequence
  Promoter; promoter lrgA
atgaaaaacgattgaatcccacttattttatacgtattcatcgttcatatattattaacacgaaacacattaaagaagtgcaacaatggtttaactacacttatatggtaatattgacaaatggtgtcaagatgcaagttggacgttcatttatgaaagattttaaagcgtcgataggattactttaacagtaatccttttttttatgcattttacctatgatattttgtatttcggactaaaaatcacgcaaatcgaagtgagccatctatactttagttaaatcaaacgtaggaggcaatg

SEQ ID NO: 141 - DNA, 303 bp, Artificial Sequence
  Promoter; promoter lrgB
gtttagtattattatttgtattattatgtactggtgctgttaagttaggcgaagtcgaaaaagtaggaacgacactaacaaataacattggcttactcttcgtaccagccggtatctcagttgttaactctttaggtgtcattagccaagcaccatttttaatcattggactaataatcgtctcaacaatactattacttatttgtactggctatgtcacacaaattattatgaaagttacttcgagatctaaaggtgacaaagtcacaaaaaagatcaaaatagaggaggcacaagctcatg

SEQ ID NO: 142 - DNA, 303 bp, Artificial Sequence
  Promoter; promoter hlgB
aagatcctagagattatttcgttccagacagtgagttacctcctcttgtacaaagtggatttaacccttcatttatcgccacagtatctcatgaaaaaggttcaagcgatacaagcgaatttgaaattacttacggaagaaacatggatgtcactcatgccattaaaagatcaacgcattatggcaacagttatttagacggacatagagtccataatgcattcgtaaatagaaactatactgttaaatacgaggtcaattggaagactcatgaaatcaaggtgaaaggacagaattgatatg

SEQ ID NO: 143 - DNA, 303 bp, Artificial Sequence
  Promoter; promoter fhuB
tcaaaatgtaacaatgatcagaggcatatgtttaattattgctatgattctagcaggtattgcagttgctatcgctggacaagttgcatttgtaggtttgatggtacctcatatagcaagatttttaattggaactgattatgctaaaattctaccattaacagccttgttaggtgggatactcgtgcttgttgccgatgtgatagcacgatatttaggagaagcgcctgttggtgcaatcatttcatttatcggtgttccttactttttatatttagttaaaaaaggaggacgctcaatatg

SEQ ID NO: 144 - DNA, 303 bp, Artificial Sequence
  Promoter; promoter splF
gttcacctatattaaatagtaagcgagaagcaattggtgttatgtatgctagtgataaaccaacaggtgaaagtacaaggtcatttgctgtttatttctctcctgaaattaagaaatttattgcagataatttagataaataaatcatccatccatacattgataaatgatttttagaaattaacaacaaaatcaacaattttaaacatctctgtgattctatttattcgaaatgatttaaaaaataaaacttcaaaaacctaaccttatatttatacgaatacttagaggagcacaaaaatg

SEQ ID NO: 145 - DNA, 303 bp, Artificial Sequence
  Promoter; promoter SAUSA300_2268
gatgatgtatgtttcgaatttatcaattaacatgtgaggacctcccgaggaatacatggcattaaatacacgtttaatatttataaaggtgacttaattttgttcaagttgattttaccacgctttttttctttattcactaagacttttgaatgaagtttaaaataattgtttatcagtgataaaatatttgcaataagaagagaatggctaaataatcttaattttcagaaaagtaattgtaaccttactggtcttatggtaatatttttcaatattatcgacgaggatgtgttaacaatg

SEQ ID NO: 146 - DNA, 303 bp, Artificial Sequence
  Promoter; promoter SAUSA300_2616
ctatcattataatgagataatgtcatttttaattgagctaaacagacagggaaagacgattattatgattacgcatgatatgcatttattgtctgagtatagttcaagaacagttgtattatcaaaaggacaagtcgttgctgataccacgccagtattgatattaaatgataaaaaaatctgtgagattgcatcattgagacaaacatcgctatttgaaatggccgaatatatagggattagcgagccacagaaattagtacaattatttattaaccatgataggaaggtgagacgccaatg

SEQ ID NO: 147 - DNA, 303 bp, Artificial Sequence
  Promoter; promoter SAUSA300_2617
caggcctattttctaggaaatcgatgatttattttaatatcggtcaaattattgcgaatattatttgctgggcacttattgcaccaacattagatattttgatttataacgaaccggctaacaaggtttatacacaaggtgttatctctgcagtattaaatattatttcagttggtattattgggacaatattattaaaagcatatgcttcatctcaaataaaaaaaggtagtttacgtaaagaataatcattttgttgaatcagatatgtaaatgaatgtagaaaggtaatgatatatcatg

SEQ ID NO: 148 - DNA, 303 bp, Artificial Sequence; identical to SEQ ID NO: 121
  Promoter; promoter isdA

SEQ ID NO: 149 - DNA, 303 bp, Artificial Sequence
  Promoter; promoter isdB
cttcagttgataactttattagcacagttgccttcgcaacacttgcccttttaggttcattatctttattacttttcaaaagaaaagaatctaaataaatcatcgtcacactcataacttaatatattttttattttaaattttatttaacctatgtcatagatatttcataatctataacataggttatttttttataaaataatgttgcaattaactaccatttcaatgtacaatacaagtaatcaattgataatgattatcagttgataatatacaattaggagttgtttctacaacatg

SEQ ID NO: 150 - DNA, 303 bp, Artificial Sequence
  Promoter; promoter fhuA/C
ctttcttgcagatgaataaataaatggtatgagcacacatacttaaatagaagtccacggacaagtttttgaactatgaagacttatctgtgggcgttttttattttataaaagtaatatacaagacatgacaaatcgagctatccaatttaaaaagtaatgttagtcaataagattgaaaaatgttataatgatgttcatgataatcattatcaattgggatgcctttgaaaattgataatttaaaaatagaaattattttttataaacagaaagaattttattgaaagtagggaaattatg

SEQ ID NO: 151 - DNA, 303 bp, Artificial Sequence
  Promoter; promoter ear
tgacacctgctaattcaaacattatttgagacattcttttcaaattaattataaatttttacctatagactagtttgatatttatctacatctcaaaattctcatcaacaatctttcacatccaacatttttactttagtttttataattcaaaacaacaaaacgatgttaaaaaattattctattttttagttaatagatagttaatacatttttgatatttagttaattgttcttttaaaaaaatattattatattttcattgtaaacgtttacaatataaaaaaaggagcaattaaaatg

SEQ ID NO: 152 - DNA, 303 bp, Artificial Sequence
  Promoter; promoter fnb
tgtacaggcgataattatgaaacacttagtatattgttttaaattagataatgatgaatttaatttgaaaaataagtataaaaaatacaagccttgtgtgacaagggtttatgatgacttgaatacaatttataggtatatttcaaataataaaattatcaattaacataaaattaatgacaatcttaacttttcattaactcgcttttttgtattgcttttaaaaaccgaacaatatagacttgcatttattaagtttaaaaaaattaatgaattttgcatttaaagggagatattatagtg

SEQ ID NO: 153 - DNA, 303 bp, Artificial Sequence
  Promoter; promoter splD
attttaaattttgatgcatacattgaacccgggaattcaggatcaccagttctaaattctaacaatgaggtcataggtgtggtgtatggcggtattggaaaaattggttctgaatataatggtgccgtatactttacgcctcaaatcaaagattttattcaaaagcacattgaacaataaacaaatttaaatatacaccatgagcatgtgttcaataattttaatgaaaaacatcggtcgaatataacataaaaaaacgtctatatcaaaagcatcatgaataaacagaggagcacaaaaatg

SEQ ID NO: 154 - DNA, 303 bp, Artificial Sequence
  Promoter; promoter dps
ataatagaaatagaatgtggaaaacaacatggcaccaaccaaatgattatgaaaaatcgttctttttagatgataatgcgaaagtaaaacttactgattgataaaacatacttgctaattgataatggatatactagatgatgaattaaaatttagacatttaaaaagcggaacaccttacatttagattagaataattataaaaaagagagtaaaaacactttacagattagaatcattataatataataattaatataaacaagcaagacgtagacaattttaaggagtgtattaaatatg

SEQ ID NO: 155 - DNA, 300 bp, Artificial Sequence
  Promoter; promoter CH52_00360
gaattctttatagcgcgtgcaatcacaccacaagataaaagattaaaaagtgacaaagcatttattgcatttttagaagaaaccttcgatcagttcttaccattttattctgcataaataactttgtttaaataatagagcacgtaatcacatccatgatttcgtgctcttttttcttaatattaaatcgaacgttcaacataataattcatacttttaaaaaaattaaaataaatttaggttgacctaaacattttattaggttattatattgtccataagaagtagaggtgagtcaaa

SEQ ID NO: 156 - DNA, 303 bp, Artificial Sequence
  Promoter; promoter CH52_00305
cataatccccctccttaaatttgttcatataagattatgatatcttagattgcataaaaagactaggtttaataaaattaaaatgtgacaaattaacgacaagagaaaatgtcaattttgtgacacaaataacatttaatttattgctataatgtatatgttagaaaattttaataagtagaatcatgcatctaaaagagattaatatttaagcttcaaatttgagtaaacgtggattacataattatcccaataaaaaaatcattacgattaagttctttttatgtcgtccacatacaatac

SEQ ID NO: 157 - DNA, 303 bp, Artificial Sequence
  Promoter; promoter CH52_01670
cattttatattccctccgtaaaatataaagttttcttaactagtttataataattttaatttgtagtcaaaaagactttgtaataatgcgttcagttaattataacttacttataccttaatataaacaacttaaaccctttttattatttttaataactctaaagtacaactctaatccgctctctttaaaaatataaatgataataagtgcacataatttctcaatggattttatgaatttaaaatatgttatcatttcactaggacatttgtaatatggtatgatgctatttatgatttt

SEQ ID NO: 158 - DNA, 302 bp, Artificial Sequence
  Promoter; promoter srtB
cataaaaatcctcttttattaacgacgtttcttcagtcatcactaaaccagttgttgtaccgttttagattcgatttcgttgactttgacaaattaagtaaattagcattggaccaccgacaatcattaaaatagcattggctggaatttctaaaggaggctgtatcactcgtcctaataaatcagccactaacaatagccatgcaccaataactgtagaaaacggaataagtactctgtaattgcccccaactagctttctaaccacatgtggcacaataatacctaaaaaggctagttgt

SEQ ID NO: 159 - DNA, 303 bp, Artificial Sequence
  Promoter; promoter sbnA
caaaagcgcttcctcctcaaatttaaaattctataatattgtgtgttacctaattgataatgattctcactatcaagtaattaggattatattttttatgcatttatatgtcaaataattataagttgcatgtaaatcataaatattttattgacttaggaaaaaatttaattcatactaaatcgtgataatgattctcattgtcatacatcacgaaggaggctaattagtcaatgaataaagtaattaaaatgcttgttgttacgcttgctttcctacttgttttagcaggatgtagtggga

SEQ ID NO: 160 - DNA, 303 bp, Artificial Sequence
  Promoter; promoter clfA
cattttattccctctttttaaaaagtcattttatattaactatataccctttaaagatatatttaatctctgttaatggaattatacactaaaattgcattatagcaattaatttgtatcgatattttattatccacaataatactttactaacaaacattttatttattgctattttaagaattacaaacgacaacgtacgatttgattgcaaacattttttattattaatatgaactctacctaatgtaatcctagctttaaatcatattttttcaaaagcagatgtgtaatttatggtac

SEQ ID NO: 161 - DNA, 303 bp, Artificial Sequence
  Promoter; promoter emp homolog
catctgttatttctcctttatatagactcaatattataaccaatataatttccctgttatattcactaacagcattatataccagaattttcagtataataattaacttgaagtaaacgttgtcttaacatttttattgtttttcagcttaaaattaattattgatattgatagttaagcataataattttttcgtaatataaagtgaaaaaagtaatagtccacacctgtttagaatgtggactatactagattgcatcattgaaatgatgactttgatattatttattgctagtttaaaat

SEQ ID NO: 162 - DNA, 283 bp, Artificial Sequence
  Promoter; promoter rsaC
cacgctgtgttttaatgaagtaagatgaattgatgttgatgcaacctaaaatattggtatctccaatattttaggctacacatcaacataacaaagtcgaaggctaatagtcccatatcgtgcgttaaatatatattaccctcctattaatatatataccgttcccgatcgcacgatatggtggtattagaacttctctttgaacgaaagagaaaagctagaacttatgcagttttaattaaactgtaaacatttgtcactctttaaatcaaagagtaaagtt

SEQ ID NO: 163 - DNA, 303 bp, Artificial Sequence
  Promoter; promoter hlgA1
aacaatttgtattttacaaacattaattaaaaataaaagcaagacattcgtgcaatcggttaccttaaattgtttacaactgtcaacaataccaaggttttattaactatatttctcacaaaattagcttttagcattccaaacaaaaaaggttaaattgaacggaattatggcatttttaacttaattgtaaaaaagttgataatggtcaattgttaatgaacagttaattataataacgtccaaaatatattattatttaattaagttaaataaaattatagaaagaaagtgaaacttatg

SEQ ID NO: 164 - DNA, 30 bp, Artificial Sequence
  Primer; forward primer isdA
tatatgcatgcctatctgcggcatttgcag

SEQ ID NO: 165 - DNA, 32 bp, Artificial Sequence
  Primer; reverse primer isdA
gatacctgcaggttgttttcctcctaaggata

SEQ ID NO: 166 - DNA, 32 bp, Artificial Sequence
  Primer; forward primer isdB
gatgcgcatgccttcagttgataactttatta

SEQ ID NO: 167 - DNA, 32 bp, Artificial Sequence
  Primer; reverse primer isdB
gatgcctgcaggttgtagaaacaactcctaat

SEQ ID NO: 168 - DNA, 32 bp, Artificial Sequence
  Primer; forward primer isdI
gatacgcatgcttactcgtagcagttttttgt

SEQ ID NO: 169 - DNA, 32 bp, Artificial Sequence
  Primer; reverse primer isdI
gatagctgcaggggcaatcactcctctatttt

SEQ ID NO: 170 - DNA, 32 bp, Artificial Sequence
  Primer; forward primer isdG
gatgcgcatgcaaacacaagataattgaattt

SEQ ID NO: 171 - DNA, 33 bp, Artificial Sequence
  Primer; reverse primer isdG
gatgcctgcagaattatcctcttttctgtttaa

SEQ ID NO: 172 - DNA, 36 bp, Artificial Sequence
  Primer; forward primer sbnC
gaatcgcatgcctttattaaagctgacaaagtcgta

SEQ ID NO: 173 - DNA, 32 bp, Artificial Sequence
  Primer; reverse primer sbnC
gaaatcctgcagtgttcagacacctcgcattc

SEQ ID NO: 174 - DNA, 51 bp, Artificial Sequence
  Primer; forward primer sbnE
taactgactaggcggccgcgaatacttcaaggattaacatatagtgcattg

SEQ ID NO: 175 - DNA, 62 bp, Artificial Sequence
  Primer; reverse primer sbnE
ccagtgaaaagttcttctcctttactcatttttttgtttcaactcccataatttcattaatg

SEQ ID NO: 176 - DNA, 53 bp, Artificial Sequence
  Primer; forward primer lrgA
taactgactaggcggccgcatgaaaaacgattgaatcccacttattttatacg

SEQ ID NO: 177 - DNA, 56 bp, Artificial Sequence
  Primer; reverse primer lrgA
ccagtgaaaagttcttctcctttactcattgcctcctacgtttgatttaactaaag

SEQ ID NO: 178 - DNA, 58 bp, Artificial Sequence
  Primer; forward primer lrgB
taactgactaggcggccgcgtttagtattattatttgtattattatgtactggtgctg

SEQ ID NO: 179 - DNA, 52 bp, Artificial Sequence
  Primer; reverse primer lrgB
ccagtgaaaagttcttctcctttactcatgagcttgtgcctcctctattttg

SEQ ID NO: 180 - DNA, 46 bp, Artificial Sequence
  Primer; forward primer hlgB
taactgactaggcggccgcaagatcctagagattatttcgttccag

SEQ ID NO: 181 - DNA, 57 bp, Artificial Sequence
  Primer; reverse primer hlgB
ccagtgaaaagttcttctcctttactcatatcaattctgtcctttcaccttgatttc

SEQ ID NO: 182 - DNA, 53 bp, Artificial Sequence
  Primer; forward primer fhuA
taactgactaggcggccgcctttcttgcagatgaataaataaatggtatgagc

SEQ ID NO: 183 - DNA, 60 bp, Artificial Sequence
  Primer; reverse primer fhuA
ccagtgaaaagttcttctcctttactcataatttccctactttcaataaaattctttctg

SEQ ID NO: 184 - DNA, 44 bp, Artificial Sequence
  Primer; forward primer fhuB
taactgactaggcggccgctcaaaatgtaacaatgatcagaggc

SEQ ID NO: 185 - DNA, 65 bp, Artificial Sequence
  Primer; reverse primer fhuB
ccagtgaaaagttcttctcctttactcatattgagcgtcctccttttttaactaaatataaaaag

SEQ ID NO: 186 - DNA, 47 bp, Artificial Sequence
  Primer; forward primer ear
taactgactaggcggccgctgacacctgctaattcaaacattatttg

SEQ ID NO: 187 - DNA, 65 bp, Artificial Sequence
  Primer; reverse primer ear
ccagtgaaaagttcttctcctttactcattttaattgctcctttttttatattgtaaacgtttac

SEQ ID NO: 188 - DNA, 48 bp, Artificial Sequence
  Primer; forward primer fnb
taactgactaggcggccgctgtacaggcgataattatgaaacacttag

SEQ ID NO: 189 - DNA, 74 bp, Artificial Sequence
  Primer; reverse primer fnb
ccagtgaaaagttcttctcctttactcattataatatctccctttaaatgcaaaattcattaatttttttaaac

SEQ ID NO: 190 - DNA, 47 bp, Artificial Sequence
  Primer; forward primer hlb
taactgactaggcggccgcttcaggctatcaataatgctttgaaatc

SEQ ID NO: 191 - DNA, 59 bp, Artificial Sequence
  Primer; reverse primer hlb
ccagtgaaaagttcttctcctttactcatagaaaccttgtaacaacagtatttattggg

SEQ ID NO: 192 - DNA, 50 bp, Artificial Sequence
  Primer; forward primer splF
taactgactaggcggccgcgttcacctatattaaatagtaagcgagaagc

SEQ ID NO: 193 - DNA, 65 bp, Artificial Sequence
  Primer; reverse primer splF
ccagtgaaaagttcttctcctttactcatttttgtgctcctctaagtattcgtataaatataagg

SEQ ID NO: 194 - DNA, 51 bp, Artificial Sequence
  Primer; forward primer splD
taactgactaggcggccgcattttaaattttgatgcatacattgaacccgg

SEQ ID NO: 195 - DNA, 58 bp, Artificial Sequence
  Primer; reverse primer splD
ccagtgaaaagttcttctcctttactcatttttgtgctcctctgtttattcatgatgc

SEQ ID NO: 196 - DNA, 52 bp, Artificial Sequence
  Primer; forward primer dps
taactgactaggcggccgcataatagaaatagaatgtggaaaacaacatggc

SEQ ID NO: 197 - DNA, 60 bp, Artificial Sequence
  Primer; reverse primer dps
ccagtgaaaagttcttctcctttactcatatttaatacactccttaaaattgtctacgtc

SEQ ID NO: 198 - DNA, 55 bp, Artificial Sequence
  Primer; forward primer SAUSA 300_2268
taactgactaggcggccgcgatgatgtatgtttcgaatttatcaattaacatgtg

SEQ ID NO: 199 - DNA, 57 bp, Artificial Sequence
  Primer; reverse primer SAUSA 300_2268
ccagtgaaaagttcttctcctttactcattgttaacacatcctcgtcgataatattg

SEQ ID NO: 200 - DNA, 57 bp, Artificial Sequence
  Primer; forward primer SAUSA 300_2616
taactgactaggcggccgcctatcattataatgagataatgtcatttttaattgagc

SEQ ID NO: 201 - DNA, 49 bp, Artificial Sequence
  Primer; reverse primer SAUSA 300_2616
ccagtgaaaagttcttctcctttactcattggcgtctcaccttcctatc

SEQ ID NO: 202 - DNA, 45 bp, Artificial Sequence
  Primer; forward primer SAUSA 300_2617
taactgactaggcggccgccaggcctattttctaggaaatcgatg

SEQ ID NO: 203 - DNA, 65 bp, Artificial Sequence
  Primer; reverse primer SAUSA 300_2617
ccagtgaaaagttcttctcctttactcatgatatatcattacctttctacattcatttacatatc

SEQ ID NO: 204 - DNA, 60 bp, Artificial Sequence
  Primer; forward primer hlgA2
cgttaactaattaatttaagaaggagatatacatacttcaaattttcacaaactattgcg

SEQ ID NO: 205 - DNA, 70 bp, Artificial Sequence
  Primer; reverse primer hlgA2
ccagtgaaaagttcttctcctttactcatagaaatcactttctttctatttaattttaagttcatatata

SEQ ID NO: 206 - DNA, 60 bp, Artificial Sequence
  Primer; forward primer hrtAB
cgttaactaattaatttaagaaggagatatacatgttcatattgagttcatatttcaacc

SEQ ID NO: 207 - DNA, 55 bp, Artificial Sequence
  Primer; reverse primer hrtAB
ccagtgaaaagttcttctcctttactcatatcgattcacttctccctatttcttc

SEQ ID NO: 208 - DNA, 29 bp, Artificial Sequence
  Primer; forward primer for pCN56 plasmid with hlgA2, hrtAB promoters
atgagtaaaggagaagaacttttcactgg

SEQ ID NO: 209 - DNA, 40 bp, Artificial Sequence
  Primer; reverse primer for pCN56 plasmid with hlgA2, hrtAB promoters
atgtatatctccttcttaaattaattagttaacgaattcg

SEQ ID NO: 210 - DNA, 29 bp, Artificial Sequence
  Primer; forward primer for pCN56 plasmid
atgagtaaaggagaagaacttttcactgg

SEQ ID NO: 211 - DNA, 39 bp, Artificial Sequence
  Primer; reverse primer for pCN56 plasmid
gcggccgcctagtcagttaactcaaaggcggtaatacgg

SEQ ID NO: 212 - DNA, 20 bp, Artificial Sequence
  Primer; forward qRT PCR primer for gyrB housekeeping
ttggtacaggaatcggtggc

SEQ ID NO: 213 - DNA, 20 bp, Artificial Sequence
  Primer; reverse qRT PCR primer for gyrB housekeeping
tccatccacatcggcatcag

SEQ ID NO: 214 - DNA, 20 bp, Artificial Sequence
  Primer; isdA forward qRT PCR primer
gcaacagaagctacgaacgc

SEQ ID NO: 215 - DNA, 22 bp, Artificial Sequence
  Primer; isdA reverse qRT PCR primer
agagccatctttttgcacttgg

SEQ ID NO: 216 - DNA, 25 bp, Artificial Sequence
  Primer; isdB forward qRT PCR primer
gcaacaattttatcattatgccagc

SEQ ID NO: 217 - DNA, 22 bp, Artificial Sequence
  Primer; isdB reverse qRT PCR primer
tggcaactttttgtcaccttca

SEQ ID NO: 218 - DNA, 21 bp, Artificial Sequence
  Primer; isdI forward qRT PCR primer
accgaggatacagacgaagtt

SEQ ID NO: 219 - DNA, 21 bp, Artificial Sequence
  Primer; isdI reverse qRT PCR primer
tgctgtccatcgtcatcactt

SEQ ID NO: 220 - DNA, 21 bp, Artificial Sequence
  Primer; isdG forward primer
aaccaatccgtaaaagcttgc

SEQ ID NO: 221 - DNA, 20 bp, Artificial Sequence
  Primer; isdG reverse qRT PCR primer
aggctttgatggcatgtttg

SEQ ID NO: 222 - DNA, 21 bp, Artificial Sequence
  Primer; sbnC forward qRT PCR primer
agggaagggtgtctaagcaac

SEQ ID NO: 223 - DNA, 20 bp, Artificial Sequence
  Primer; sbnC reverse qRT PCR primer
tcagtccttcttcaacgcga

SEQ ID NO: 224 - DNA, 20 bp, Artificial Sequence
  Primer; sbnE forward qRT PCR primer
attcgctttagccgcaatgg

SEQ ID NO: 225 - DNA, 20 bp, Artificial Sequence
  Primer; sbnE reverse qRT PCR primer
gcaacttgtagcgcatcgtc

SEQ ID NO: 226 - DNA, 21 bp, Artificial Sequence
  Primer; lrgA forward qRT PCR primer
gataccggctggtacgaagag

SEQ ID NO: 227 - DNA, 21 bp, Artificial Sequence
  Primer; lrgA reverse qRT PCR primer
tggtgctgttaagttaggcga

SEQ ID NO: 228 - DNA, 20 bp, Artificial Sequence
  Primer; lrgB forward qRT PCR primer
acaaagacaggcacaactgc

SEQ ID NO: 229 - DNA, 20 bp, Artificial Sequence
  Primer; lrgB reverse qRT PCR primer
ggtgtagcaccagccaaaga

SEQ ID NO: 230 - DNA, 21 bp, Artificial Sequence
  Primer; hlgB forward qRT PCR primer
tggttggggaccttatggaag

SEQ ID NO: 231 - DNA, 20 bp, Artificial Sequence
  Primer; hlgB reverse qRT PCR primer
ggcatttggtgttgcgctat

SEQ ID NO: 232 - DNA, 21 bp, Artificial Sequence
  Primer; fhuA forward qRT PCR primer
cacgttgtctttgaccaccac

SEQ ID NO: 233 - DNA, 21 bp, Artificial Sequence
  Primer; fhuA reverse qRT PCR primer
tgggcaatggaagttacagga

SEQ ID NO: 234 - DNA, 20 bp, Artificial Sequence
  Primer; fhuB forward qRT PCR primer
caatacctgctggaacccca

SEQ ID NO: 235 - DNA, 20 bp, Artificial Sequence
  Primer; fhuB reverse qRT PCR primer
gggtccgcatattgccaaac

SEQ ID NO: 236 - DNA, 21 bp, Artificial Sequence
  Primer; ear forward qRT PCR primer
ccacttgtcagatctgctcct

SEQ ID NO: 237 - DNA, 24 bp, Artificial Sequence
  Primer; ear reverse qRT PCR primer
ggtttggttacagatggacaaaca

SEQ ID NO: 238 - DNA, 19 bp, Artificial Sequence
  Primer; fnb forward qRT PCR primer
cgcagtgagcgaccataca

SEQ ID NO: 239 - DNA, 20 bp, Artificial Sequence
  Primer; fnb reverse qRT PCR primer
ttggtccttgtgcttgacca

SEQ ID NO: 240 - DNA, 20 bp, Artificial Sequence
  Primer; hlb forward qRT PCR primer
ctacgccaccatcttcagca

SEQ ID NO: 241 - DNA, 20 bp, Artificial Sequence
  Primer; hlb reverse qRT PCR primer
acacctgtactcggtcgttc

SEQ ID NO: 242 - DNA, 22 bp, Artificial Sequence
  Primer; splF forward qRT PCR primer
tgcaattattcagcctggtagc

SEQ ID NO: 243 - DNA, 22 bp, Artificial Sequence
  Primer; splF reverse qRT PCR primer
cctgatggcttattaccggcat

SEQ ID NO: 244 - DNA, 20 bp, Artificial Sequence
  Primer; splD forward qRT PCR primer
agtgacatctgatgcggttg

SEQ ID NO: 245 - DNA, 21 bp, Artificial Sequence
  Primer; splD reverse qRT PCR primer
aacaccaattgcttctcgctt

SEQ ID NO: 246 - DNA, 20 bp, Artificial Sequence
  Primer; dps forward qRT PCR primer
agcggtaggaggaaaccctg

SEQ ID NO: 247 - DNA, 22 bp, Artificial Sequence
  Primer; dps reverse qRT PCR primer
gttctgcagagtaacctttcgc

SEQ ID NO: 248 - DNA, 21 bp, Artificial Sequence
  Primer; srtB  forward qRT PCR primer
tgagcgagaacatcgacgtaa

SEQ ID NO: 249 - DNA, 20 bp, Artificial Sequence
  Primer; srtB reverse qRT PCR primer
ccgacatggtgcccgtataa

SEQ ID NO: 250 - DNA, 21 bp, Artificial Sequence
  Primer; emp  forward qRT PCR primer
tcgcgtgaatgtagcaacaaa

SEQ ID NO: 251 - DNA, 21 bp, Artificial Sequence
  Primer; emp  reverse qRT PCR primer
acttctgggcctttagcaaca

SEQ ID NO: 252 - DNA, 20 bp, Artificial Sequence
  Primer; sbnA  forward qRT PCR primer
cctggaggcagcatgaaaga

SEQ ID NO: 253 - DNA, 20 bp, Artificial Sequence
  Primer; sbnA  reverse qRT PCR primer
cattgccaacgcaatgccta

SEQ ID NO: 254 - DNA, 20 bp, Artificial Sequence
  Primer; CH52_360 forward qRT PCR primer
ttcaactcgaacgctgacga

SEQ ID NO: 255 - DNA, 20 bp, Artificial Sequence
  Primer; CH52_360 reverse qRT PCR primer
ttgcacccattgttgcacct

SEQ ID NO: 256 - DNA, 20 bp, Artificial Sequence
  Primer; CH52_305 forward qRT PCR primer
ttcctggagcagtaccacca

SEQ ID NO: 257 - DNA, 22 bp, Artificial Sequence
  Primer; CH52_305 reverse qRT PCR primer
cagcgcaatcgctgttaaacta

SEQ ID NO: 258 - DNA, 20 bp, Artificial Sequence
  Primer; CH521670 forward qRT PCR primer
gcgattatgggaccaaacgg

SEQ ID NO: 259 - DNA, 21 bp, Artificial Sequence
  Primer; CH521670 reverse qRT PCR primer
acttcatagcttgggtgtccc

SEQ ID NO: 260 - DNA, 20 bp, Artificial Sequence
  Primer; clfA  forward qRT PCR primer
tccagcacaacaggaaacga

SEQ ID NO: 261 - DNA, 20 bp, Artificial Sequence
  Primer; clfA  reverse qRT PCR primer
tagcttcaccagttaccggc

SEQ ID NO: 262 - DNA, 20 bp, Artificial Sequence
  Primer; SAUSA300_2268 forward qRT PCR primer
gcttctacagctttgccgat

SEQ ID NO: 263 - DNA, 21 bp, Artificial Sequence
  Primer; SAUSA300_2268 reverse qRT PCR primer
gatttggtgcttactgccacc

SEQ ID NO: 264 - DNA, 20 bp, Artificial Sequence
  Primer; SAUSA300_2616 forward qRT PCR primer
acaagcgcaacaagcaagag

SEQ ID NO: 265 - DNA, 23 bp, Artificial Sequence
  Primer; SAUSA300_2616 reverse qRT PCR primer
tgcgtttgatacctttaacacgg

SEQ ID NO: 266 - DNA, 21 bp, Artificial Sequence
  Primer; SAUSA300_2617 forward qRT PCR primer
gggctgaaaaagttggcatga

SEQ ID NO: 267 - DNA, 20 bp, Artificial Sequence
  Primer; SAUSA300_2617 reverse qRT PCR primer
acgcgttgtttttgacctcc

SEQ ID NO: 268 - DNA, 21 bp, Artificial Sequence
  Primer; hlgA2 forward qRT PCR primer
tgatttctgcaccttgaccga

SEQ ID NO: 269 - DNA, 20 bp, Artificial Sequence
  Primer; hlgA2 reverse qRT PCR primer
agcccctttagccaatccat

SEQ ID NO: 270 - DNA, 22 bp, Artificial Sequence
  Primer; hrtAB  forward qRT PCR primer
acacaacaacaacgtgatgagc

SEQ ID NO: 271 - DNA, 20 bp, Artificial Sequence
  Primer; hrtAB  reverse qRT PCR primer
taacggtgcttgctctgctt

SEQ ID NO: 272 - DNA, 204 bp, Staphylococcus aureus
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttgt

SEQ ID NO: 273 - DNA, 60 bp, Staphylococcus aureus
cccctcactaccgcaaatagtgaggggattggtgtataagtaaatacttattttcgttgt

SEQ ID NO: 274 - DNA, 144 bp, Staphylococcus aureus
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaat

SEQ ID NO: 275 - DNA, 120 bp, Staphylococcus aureus
atgcttattttcgttcacatcatagcaccagtcatcagtggctgtgccattgcgtttttttcttattggctaagtagacgcaatacaaaataggtgacatatagccgcaccaataaaaat

SEQ ID NO: 276 - DNA, 31 bp, Artificial Sequence
  BPC_670 oligo for plasmid construction
gctcagatctgttaacggtaccatcatactc

SEQ ID NO: 277 - DNA, 21 bp, Artificial Sequence
  BPC_671 oligo for plasmid construction
cactggccgtcgttttacaac

SEQ ID NO: 278 - DNA, 55 bp, Artificial Sequence
  BPC_672 oligo for plasmid construction
gagtatgatggtaccgttaacagatctgagccgcagagaggaggtgtataaggtg

SEQ ID NO: 279 - DNA, 60 bp, Artificial Sequence
  BPC_674 oligo for plasmid construction
gagtatgatggtaccgttaacagatctgagcatggtggcattactgaaatctttagaaag

SEQ ID NO: 280 - DNA, 51 bp, Artificial Sequence
  BPC_675 oligo for plasmid construction
gagtatgatggtaccgttaacagatctgagcatggcactgcctaaaacggg

SEQ ID NO: 281 - DNA, 61 bp, Artificial Sequence
  BPC_676 oligo for plasmid construction
gagtatgatggtaccgttaacagatctgagcatggctaatgaaactaaacaacctaaagtt

SEQ ID NO: 282 - DNA, 41 bp, Artificial Sequence
  BPC_677 oligo for plasmid construction
gttgtaaaacgacggccagtgcccgggctcagctattatca

SEQ ID NO: 283 - DNA, 39 bp, Artificial Sequence
  BPC_678 oligo for plasmid construction
gttgtaaaacgacggccagtggcggccgcccatgcatgc

SEQ ID NO: 284 - DNA, 93 bp, Staphylococcus aureus
atgcttattttcgttcacatcatagcaccagtcatcagtggctgtgccattgcgtttttttcttattggctaagtagacgcaatacaaaatag

SEQ ID NO: 285 - protein, 29 aa, Staphylococcus aureus
LIFVHIIAPVISGCAIAFFSYWLSRRNTK

SEQ ID NO: 286 - DNA, 795 bp, Artificial Sequence
  187-lysK toxin gene encodes engineered phage lysin protein from the Staphylococcus aureus phage
atggcactgcctaaaacgggtaaaccaacggcaaaacaggtggttgactgggcaatcaatttaatcggcagtggtgtcgatgttgatggttattatggtcggcaatgttgggatttacctaactatatttttaatagatactggaactttaagacaccaggcaacgcaagagatatggcatggtatagatatcctgaagggtttaaagtgtttagaaacacttctgattttgtccctaaaccaggtgatatagcagtgtggacaggtggtaattacaattggaacacttggggacacactggtattgttgtaggtccatcaactaaaagttacttttatagtgtagatcagaattggaataactctaactcttacgttggtagtcctgcagcaaagataaaacatagttattttggtgtaactcattttgttagacccgcatacaaagcagaaccgaaacctacaccaccactggacagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaa

SEQ ID NO: 287 - protein, 265 aa, Artificial Sequence
  recombinant phage lysin LysK toxin
MALPKTGKPTAKQVVDWAINLIGSGVDVDGYYGRQCWDLPNYIFNRYWNFKTPGNARDMAWYRYPEGFKVFRNTSDFVPKPGDIAVWTGGNYNWNTWGHTGIVVGPSTKSYFYSVDQNWNNSNSYVGSPAAKIKHSYFGVTHFVRPAYKAEPKPTPPLDSTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 288 - DNA, 504 bp, Staphylococcus aureus
atggctaatgaaactaaacaacctaaagttgttggaggaataaactttagcacaagaactaagagtaaaacattttgggtagcaattatatcagcagtagcagtatttgctaatcaaattacaggtgcttttggtttagactactcagctcaaattgagcaaggtgtaaatatcataggttctatactaacattattagcaggtttaggtattattgttgataataatactaaaggtcttaaagatagtgatattgttcaaacagattatataaaacctcgtgatagtaaagaccctaatgaatttgttcaatggcaagcaaatgcaaacacagctagcactttcgaattagacaactatgaaaacaatgcagaacctgatacagatgatagtgatgaagtacctgctattgaagatgaaattgatggcggttcagcaccttctcaagatgaagaagataccgaggaacacggtaaagtatttgcagaggaggaagttaagtag

SEQ ID NO: 289 - protein, 167 aa, Staphylococcus aureus
MANETKQPKVVGGINFSTRTKSKTFWVAIISAVAVFANQITGAFGLDYSAQIEQGVNIIGSILTLLAGLGIIVDNNTKGLKDSDIVQTDYIKPRDSKDPNEFVQWQANANTASTFELDNYENNAEPDTDDSDEVPAIEDEIDGGSAPSQDEEDTEEHGKVFAEEEVK

SEQ ID NO: 290 - DNA, 135 bp, Staphylococcus aureus
atggtggcattactgaaatctttagaaaggagacgcctaatgattacaattagtaccatgttgcagtttggtttattccttattgcattgataggtctagtaatcaagcttattgaattaagcaataaaaaataa

SEQ ID NO: 291 - protein, 44 aa, Staphylococcus aureus
MVALLKSLERRRLMITISTMLQFGLFLIALIGLVIKLIELSNKK

SEQ ID NO: 292 - DNA, 20 bp, Artificial Sequence
  Primer; gfp primer
ctgtccacacaatctgccct

SEQ ID NO: 293 - DNA, 20 bp, Artificial Sequence
  Primer; gfp primer
tgccatgtgtaatcccagca

SEQ ID NO: 294 - DNA, 25 bp, Artificial Sequence
  Primer; DR_022 primer
caagcttatcgataccgtcgacctc

SEQ ID NO: 295 - DNA, 23 bp, Artificial Sequence
  Primer; DR_023 primer
gggatccactagttctagagcgg

SEQ ID NO: 296 - DNA, 31 bp, Artificial Sequence
  Primerr; DR_237 primer
gcaactggtacatcacaattggtactctcac

SEQ ID NO: 297 - DNA, 32 bp, Artificial Sequence
  Primer; DR_238 primer
gaccacgcatacctatctataaacggacaatg

SEQ ID NO: 298 - DNA, 44 bp, Artificial Sequence
  Primer; DR_255 primer
gtccaattagatggcatgtaactgggcagtgtcttaaaaaatcg

SEQ ID NO: 299 - DNA, 55 bp, Artificial Sequence
  Primer; DR_241 primer
caggccaatttggcatagagccggatgtgctgcaaggcgattaagttgggtaacg

SEQ ID NO: 300 - DNA, 45 bp, Artificial Sequence
  Primer; DR_256 primer
gttacatgccatctaattggacaaattctatgagagtagattttg

SEQ ID NO: 301 - DNA, 33 bp, Artificial Sequence
  Primer; DR_257 primer
gccaaatcgctttcgtgtatacgattcccagtc

SEQ ID NO: 302 - DNA, 30 bp, Artificial Sequence
  Primer; DR_240 primer
ggctctatgccaaattggcctgatgagttc

SEQ ID NO: 303 - DNA, 53 bp, Artificial Sequence
  Primer; DR_236 primer
gctctagaactagtggatcccggcgattttattgtgacaagagactgaagagc

SEQ ID NO: 304 - DNA, 108 bp, Staphylococcus aureus
atgttcaatttattaattaacatcatgacttcagctttaagcggctgtcttgttgcgttttttgcacattggttacgaacgcgcaacaataaaaaaggtgacaaataa

SEQ ID NO: 305 - protein, 35 aa, Staphylococcus aureus
MFNLLINIMTSALSGCLVAFFAHWLRTRNNKKGDK

SEQ ID NO: 306 - DNA, 77 bp, Staphylococcus aureus
tataattaattacataataaattgaacatctaaatacaccaaatcccctcactactgccatagtgaggggatttatt

SEQ ID NO: 307 - DNA, 140 bp, Staphylococcus aureus
atatatagaaaaagggcaacatgcgcaaacatgttaccctaatgagcccgttaaaaagacggtggctattttagattaaagattaaattaataaccatttaaccatcgaaaccagccaaagttagcgatggttatttttt

SEQ ID NO: 308 - DNA, 76 bp, Artificial Sequence
  holin antitoxin gene
tataattgagatagtttcattagctatttacttatacaccaatcccctcactatttgcggtagtgaggggattttt

SEQ ID NO: 309 - DNA, 76 bp, Artificial Sequence
  187-lysK antitoxin gene
tataattgagattttaggcagtgctatttacttatacaccaatcccctcactatttgcggtagtgaggggattttt

SEQ ID NO: 310 - DNA, 140 bp, Artificial Sequence; identical to SEQ ID NO: 307
  sprG antitoxin gene (sprF)

SEQ ID NO: 311 - DNA, 74 bp, Staphylococcus aureus
tataattgagataacgaaaataagtatttacttatacaccaatcccctcactatttgcggtagtgaggggattt

SEQ ID NO: 312 - DNA, 90 bp, Staphylococcus aureus
tataattaattacataataaattgaacatctaaatacaccaaatcccctcactactgccatagtgaggggatttatttaggtgttggtta

SEQ ID NO: 313 - DNA, 158 bp, Staphylococcus aureus
atgattatcactagccctacagaagcgagaaaagatttttatcaattactaaaaaatgttaataataatcacgaaccaatttatattagtggcaataatgccgaaaataatgctgtgattataggtttagaagattggaaaagtatacaagagacaat

SEQ ID NO: 314 - protein, 85 aa, Staphylococcus aureus
MIITSPTEARKDFYQLLKNVNNNHEPIYISGNNAENNAVIIGLEDWKSIQETIYLESTGTMDKVREREKDNSGTTNIDDIDWDNL

SEQ ID NO: 315 - DNA, 159 bp, Staphylococcus aureus
atgagcaattacacggttaagattaaaaattcagcgaaatcagatttaaggaaaataaaacattcttatttaaagaagtcatttttagaaattgttgagactttaaaaaatgatccgtataaaataacacaatcttttgaaaaattagagcctaaatat

SEQ ID NO: 316 - protein, 88 aa, Staphylococcus aureus
MSNYTVKIKNSAKSDLRKIKHSYLKKSFLEIVETLKNDPYKITQSFEKLEPKYLERYSRRINHQHRVVYTVDDRNKEVLILSAWSHYD

SEQ ID NO: 317 - DNA, 738 bp, Staphylococcus simulans
atgacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 318 - protein, 245 aa, Staphylococcus simulans
MTHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 319 - DNA, 76 bp, Artificial Sequence
  proposed lysostaphin antitoxin gene; encodes RNA antitoxin for lysostaphin
tataattgagatatgttcatgtgttatttacttatacaccaatcccctcactatttgcggtagtgaggggattttt

SEQ ID NO: 320 - DNA, 157 bp, Staphylococcus aureus
atgattagacgaggagatgtttatttagcagatttatcaccagtacagggatctgaacaagggggagtcagacctgtagtcataattcaaaatgatactggtaataaatatagtcctacagttattgttgcggcaataactggtaggattaataaag

SEQ ID NO: 321 - protein, 120 aa, Staphylococcus aureus
MIRRGDVYLADLSPVQGSEQGGVRPVVIIQNDTGNKYSPTVIVAAITGRINKAKIPTHVEIEKKKYKLDKDSVILLEQIRTLDKKRLKEKLTYLSDDKMKEVDNALMISLGLNAVAHQKN

SEQ ID NO: 322 - DNA, 158 bp, Staphylococcus aureus
atgttatcttttagtcaaaatagaagtcatagcttagaacaatctttaaaagaaggatattcacaaatggctgatttaaatctctccctagcgaacgaagcttttccgatagagtgtgaagcatgcgattgcaacgaaacatatttatcttctaattc

SEQ ID NO: 323 - protein, 56 aa, Staphylococcus aureus
MLSFSQNRSHSLEQSLKEGYSQMADLNLSLANEAFPIECEACDCNETYLSSNSTNE

SEQ ID NO: 324 - DNA, 300 bp, Staphylococcus aureus
aatggcatggatgctcaaacatatggttctcaaggacagcaacgtacaacggctttgtccattaaattagctgaaattgagttaatgaatatcgaagttggggaatatcccatcttattattagacgatgtactcagtgaattagatgattcgcgtcaaacgcatttattaagtacgattcagcataaagtacaaacatttgtcactacgacatctgtagatggtattgatcatgaaatcatgaataacgctaaattgtatcgtattaatcaaggtgaaattataaagtaacagaaagcg

SEQ ID NO: 325 - DNA, 300 bp, Staphylococcus aureus
caaatgcagttaaacatgcatacaaagaaaataacaatgtgggcattattaacatatattttgaaattttagaagataaaattaaaattgttatttctgataaaggtgacagttttgattatgaaacaactaaatcaaaaataggtccttacgataaagacgaaaatatagactttttacgcgaaggtggcctaggtttatttttaatcgaatctttaatggatgaagtcacagtatataaagaatctggtgtgacaatcagtatgactaagtatataaaaaaagagcaggtgcgaaata

SEQ ID NO: 326 - DNA, 300 bp, Staphylococcus aureus
tatggtattgaagagttcttagaagtgaaatctatagctggatattttaaataaatttgatttttgaattaaaaatcgcaataaaacagtgcacatgactaattaagttttgtgtactgttttaattttgcaatttttataaatagattttgtaattaaaataaaaatttgctatagttattcatgtatttaaaaggttggggattagcataatgggattgtgctagcacagttatttatgcattgtcatgcctatctattacttactaactaaaaaataatgaaatgggtgtaaactat

SEQ ID NO: 327 - DNA, 243 bp, Artificial Sequence
  Promoter pTK3 forward
ttgcgaaatccattcctcttccactacaagcaccataattaaacaacaattcaatagaataagacttgcaaaacatagttatgtcgctatataaacgcctgcgaccaataaatcttttaaacataacataatgcaaaaacatcatttaacaatgctaaaaatgtctcttcaatacatgttgatagtaattaacttttaacgaacagttaattcgaaaacgcttacaaatggattattatatat

SEQ ID NO: 328 - DNA, 243 bp, Artificial Sequence
  Promoter pTK3 reverse
aacgctttaggtaaggagaaggtgatgttcgtggtattaatttgttgttaagttatcttattctgaacgttttgtatcaatacagcgatatatttgcggacgctggttatttagaaaatttgtattgtattacgtttttgtagtaaattgttacgatttttacagagaagttatgtacaactatcattaattgaaaattgcttgtcaattaagcttttgcgaatgtttacctaataatatata

SEQ ID NO: 329 - DNA, 31 bp, Artificial Sequence
  Primer TKO1
gatgcgcatgcttgcgaaatccattcctctt

SEQ ID NO: 330 - DNA, 52 bp, Artificial Sequence
  Primer DR_233
cgacggtatcgataagcttggccactggcgtcaaatactgtaatgaagaatg

SEQ ID NO: 331 - DNA, 46 bp, Artificial Sequence
  Primer DR_296
catctaattggacaaattctatgagagtagattttgttaatttaag

SEQ ID NO: 332 - DNA, 38 bp, Artificial Sequence
  Primer DR_280
gtagacgcaatacaaaataggtgacatatagccgcacc

SEQ ID NO: 333 - DNA, 53 bp, Artificial Sequence; identical to SEQ ID NO: 303
  Primer DR_236

SEQ ID NO: 334 - DNA, 57 bp, Artificial Sequence
  Primer DR_297
catagaatttgtccaattagatgtcccactacatcctgctaaaacaagtaggaaagc

SEQ ID NO: 335 - DNA, 32 bp, Artificial Sequence
  Primer DR_228
ctattttgtattgcgtctacttagccaataag

SEQ ID NO: 336 - DNA, 25 bp, Artificial Sequence
  Primer DR_022
caagcttatcgataccgtcgacctc

SEQ ID NO: 337 - DNA, 23 bp, Artificial Sequence
  Primer DR_023
gggatccactagttctagagcgg

SEQ ID NO: 338 - DNA, 29 bp, Artificial Sequence
  Primer DR_303
caagccaccaaagcacgtgcctatttgcc

SEQ ID NO: 339 - DNA, 48 bp, Artificial Sequence
  Primer DR_304
cagtgaaatagatagattggttgaaaaacaatcttcaaaagtcggacg

SEQ ID NO: 340 - DNA, 980 bp, Artificial Sequence
  BP_DNA_001 isdB Upstream Homology Arm w/ Frameshift
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaataataaatc

SEQ ID NO: 341 - DNA, 950 bp, Artificial Sequence
  BP_DNA_002 isdB Downstream Homology Arm
gtctttatatttaattattaaattaacaaattttaattggcggatgaggtatccagttacctcgttcgccaattatttttcgcaatataaaaagtcccacttaaaacaatcattttaagcgggactttttatattgagtaactaaaattatttagctgctacttcttcgccattgtaagaaccacagtttttacatacacggtgtgataatttgtattcgccacagtttgggcattcagtcatacctggtactgaaattttgaaatgcgtacgacgtttgttttttctagttttagaagttcttctttttggtactgccatgatatatcctccttagattataaacgaaaaatactaaatgttagtttaattaacaacattatatcattaattaaactacttattgctctttatcatataattgttgtaatttttgaagccttggatcaacttgtcgtgattctgaatcatcttgttcttgctgtttagcaagctcatctaattgatcctcatcgattacttcccaaccattacctactgtcaacatttggtcactttgctctgaataagctctcattggtttctcaataataactatatcctcgacaatatcctgaagattaaccataccatctttaataatgtgatagtgttcatctacatcatcttgatcatcgttatactgattgtacccttctaaatcaaatacttctgtagtagttacatctagtgggacttttactggtacaagagtacgtgcacaaggcattgtatacgttccagtaatgtgaatatccgcaacgacttctgttgacttaatggttaactgaccttggattgtaattggagataaatcaattaaatctaatgattcttttaaattgtcaaaactcaccgtttgatcaaattcaaatggcttaccttgatatttccttaattgcgtaattgac

SEQ ID NO: 342 - DNA, 117 bp, Artificial Sequence
  BP_DNA_003 Fragment containing sprA1
cgcagagaggaggtgtataaggtgatgcttattttcgttcacatcatagcaccagtcatcagtggctgtgccattgcgtttttttcttattggctaagtagacgcaatacaaaatag

SEQ ID NO: 343 - DNA, 1000 bp, Artificial Sequence
  BP_DNA_004 Site_2 Upstream Homology Arm
agatattgccaatttatctattcatcttcgtacacgtaaagaaatgagtaaagtagatgcacattgggaattaatcgaagctattaaaaatttacgtgacgaaattgcaccaaatacattgttaacaattaacggtgatattcccgatagaaaaacaggacttgaactggcagaaaaatatggcatcgatggtgtcatgattggtagaggcattttccataatccattcgcttttgaaaaagaaccacgcgaacacacaagcaaggaactattagatctattgagattgcatttatcattgtttaacaaatatgaaaaagatgaaatacgacaattcaagagcttgcgtagattctttaaaatctatgtgcgtggcataagaggcgctagcgaacttcgccatcaattgatgaacacacaatcaattgcagaagcacgagcacttctagatgaatttgaagcccaaatggacgaagacgttaaaatagaattatagtatgagtaataaagtttatggatgatatttcccaatttaacacggattgaacactttctacttaggtattatcttggttttcctgataggataactcccgatgatgcctttttacttatgtcacttatccttttggaaatggtgcaaaattaaaaatacatcaggatatcgtttaactcagaaaaaaactccaatcaaaaaaaatgtttacaacacattatgaaattaaaattgctcttagtgttcaacagacaaagttattgaaattactattggaggtacataccaagctatcattttgagtacacgtggtttgaatatctggcaagaaatggaaattggtaatgtcagtcataaagtcgctaaacgtgctcaaataccattaattatttttaaataatttagaggatgaggtataaacctttaaaaaacagcagtgagatgattttcaattagaaaatatcttactgctgtttctattttcataatcattcttattgaatg

SEQ ID NO: 344 - DNA, 446 bp, Artificial Sequence
  BP_DNA_005 sprA1(AS) insertion in SA Site_2
cagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattgagataacgaaaataagtatttacttatacaccaatcccctcactatttgcggtagtgaggggatttttattggtgcggctatatgtcacctattttgtattgcgtctacttagcc

SEQ ID NO: 345 - DNA, 950 bp, Artificial Sequence
  BP_DNA_006 Site_2 Downstream Homology Arm
acttcatcgtgatcatttaaaacagtaacaactttttcaatcttatactatttagagctaaaaattcacgacatataaacacaacactgacacgtcatttaaatacttaatcttttatagtaaaagatttttcattaaaaatttattgttttctgtactttattatattaatccttttagcatgccgtaccaatacattttaggtaataaatctttcttaaatatatacatacttctacgttctttggcctgattaaacggcattgtttctttagtatttttattataatcaaactctgcaagtattaacctattatatccagtaacaatagggcatgaagtataaccatcataatgatgcgttaacatttgattattcatcacttgcaataaattattagcgacgataggtgcttgcttacgaatagctgcgcctgtttttgaagtaggtacatttgaagcatcaccaagtgcaaatacattagagtagcttttatgctgtaatgtggttgggttaacatctacccaaccctcactatctgaaagtgtactttcttttactacatctaagggacccataggtggtgttacatgtaacatatcataacttattgtttttctatcgtatgctttgatatgttcgaatgtagccacttttttgtcaccgtcgatttcaacaaggttataattataattgactgttatatttctttcttcaacaatcctttctaattctttattatattttcctacgtcaaataaagcatcttttggcgttgcatatatcacattagcgttagaacggattttatgtttcctaaaataatcttcagctaaatacataattttcataggcgcacctccacactttataggagtgtttggatgcgtaaaaatggcatttccttgtttaaaattagaaatttggttccaagtttcgttaacatagt

SEQ ID NO: 346 - DNA, 909 bp, Artificial Sequence
  BP_DNA_007 PsbnA Downstream Homology Arm
acgatcgcctcgatctggtaaaatcgtgacaattgttgcaccttcttcaattgacgttatcaactgctcaatcgctgcaataatcgagcctgttgaacctccggcaaatatgccttcataatcaatcagttttcgacagcccaaagcagattgataatcatctacatggatcacttgattaatttctgatctattcaatatttcgggtacacgactagcaccgataccaggtaattctctattaataggtttgtcaccaaaaatgactgaccctttcgcatcaacagcaacaatttgtgcgtttggatgcacttcttttatttttctactcatacccataatgctacctgtcgtgctgactggcgcgacaaaataatctataggttgcttaattgtttcaacaatctctgtgcctgcaccatgataatgggattgccaatttaactcattcgcatattgattaatccaatatgcatcgtcaatagtggctaacagttcttgcacctttgcaatacgagtcattaaataacccccatgtgcatcaggttcttcaaccatttctacattggcaccataacttttaataattttcaaatttgttggtgatattttaggatcaacaacacacgtgagttttaatcccttgattttagctatcattgccaacgcaatgcctaaattaccagaagtactttcaattaaatgtgtattctcagtgattaaaccatgtttaataccatgttcaatgatgtacttggcaggtcgatctttcatgctgcctccaggattcatatactctaactttgcaaacacttcatgtttcggaaatagttgatgaagttgaaccataggtgtttgccctacagaatctaacaatgaatcgtgacatgcttgacttttttcaatcaa

SEQ ID NO: 347 - DNA, 131 bp, Artificial Sequence
  Staphylococcus aureus Fragment containing sprA2
cgcagagaggaggtgtatgatggatgttcaatttattaattaacatcatgacttcagctttaagcggctgtcttgttgcgttttttgcacattggttacgaacgcgcaacaataaaaaaggtgacaaataa

SEQ ID NO: 348 - DNA, 976 bp, Artificial Sequence
  BP_DNA_010 isdB Upstream Homology Arm for Triple stop insertion (p260)
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaat

SEQ ID NO: 349 - DNA, 1063 bp, Artificial Sequence
  BP_DNA_012 Upstream HA (Delta PsprA1)
gccactggcgtcaaatactgtaatgaagaatgtggtctaaaattgttgtaccaatttacataatcaaataattctgtttctaactgttctaagttttcaaattgcatttgttttacaaattctgttttcattgctttcatcgttgcttccgcaactgcgttatcataaggacaacctttggtacttaatgaacgttttattttaaatgtttctaggacttcatctatcaaatgattatcaaattctttgtctctatcagtgtggaatagtttgatttgttcaagattatgatttattctgctgattgcttttgatactaaattggcgtccttatttttacctgcactgtaaccaacaatttctctattaaatagatctataaataaacatatgtaatgccatgttcctccgacttttacatatgtcaaatcacttactaatgtctccattggttgttctctattaaaagcgcgattcaaatgatttttaattcgtttttcattagtttctatttgatgatttttgtatttagctttcatataaacagaaactagattttttcataatcgacctatcttttgtccagatacagtgagacccttgtcatttaaatgatttttaattcgtcttgtactaaagacttttctattagaattaaaaatatttatggcggcacgttctacgtttgaatcatctttagtgattttattatcttttctttttatagaatcataataggtacttcttggtatttttaggactttacacattgctggtactgaatattgatgtgcattcttttgaatgacttctatttttgccccataatcagctctttttcttcatctgttaagttatcttggtgattgaatgtacccttgttttgatgttactttatccattttcctaacgtcaaaggtgttaaatcatactcgcgtataatttcatttcttggcttaccattttcatataatctaaccatttataacttaaactctgaactaaatgttcttctttcttaaattaacaaaatctactctcatagaatttgtccaattagatg

SEQ ID NO: 350 - DNA, 1063 bp, Artificial Sequence; identical to SEQ ID NO: 349
  BP_DNA_013 Upstream Homology Arm

SEQ ID NO: 351 - DNA, 1195 bp, Artificial Sequence
  BP_DNA_014 Downstream Homology Arm
cagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttatctcaattatactgctaatctttctctttgtaaaatatgcaaggttttaaagagaaacatcaagaactaaaaaaggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 352 - DNA, 695 bp, Artificial Sequence
  BP_DNA_016 uidA Upstream Homology Arm
ggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctacggtgtactggccgatatcgaagcggaagacctggcgcgtgaagcgtcgtttgctcagggattacgcgcgatgattggcggtatcttaaccgcatcctgattctctctcttttcggcgggctggtgataactgtgcccgcgtttcatatcgtaatttctctgtgcaaaaattatccttcccggcttcggagaattccccccaaaatattcactgtagccatatgtcatgagagtttatcgttcccaatacgctcgaacgaacgttcggttgcttattttatggcttctgtcaacgctgttttaaagattaatgcgatctatatcacgctgtgggtattgcagtttttggttttttgatcgcggtgtcagttctttttatttccatttctcttccatgggtttctcacagataactgtgtgcaacacag

SEQ ID NO: 353 - DNA, 672 bp, Artificial Sequence
  BP_DNA_017 uidA Downstream Homology Arm
atcaacaactctcctggcgcaccatcgtcggctacagcctcggtgacgtcgccaataacttcgccttcgcaatgggggcgctcttcctgttgagttactacaccgacgtcgctggcgtcggtgccgctgcggcgggcaccatgctgttactggtgcgggtattcgatgccttcgccgacgtctttgccggacgagtggtggacagtgtgaatacccgctggggaaaattccgcccgtttttactcttcggtactgcgccgttaatgatcttcagcgtgctggtattctgggtgctgaccgactggagccatggtagcaaagtggtgtatgcatatttgacctacatgggcctcgggctttgctacagcctggtgaatattccttatggttcacttgctaccgcgatgacccaacaaccacaatcccgcgcccgtctgggcgcggctcgtgggattgccgcttcattgacctttgtctgcctggcatttctgataggaccgagcattaagaactccagcccggaagagatggtgtcggtataccatttctggacaattgtgctggcgattgccggaatggtgctttacttcatctgcttcaaatcgacgcgtgagaatgtggtacgtatcgttgcgcagccgtcattgaatatcagtctgcaaaccctgaaac

SEQ ID NO: 354 - DNA, 329 bp, Artificial Sequence
  BP_DNA_019 PgyrB
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatat

SEQ ID NO: 355 - DNA, 83 bp, Artificial Sequence
  BP_DNA_020 sprA1(AS)(long)
ataacgaaaataagtatttacttatacaccaatcccctcactatttgcggtagtgaggggatttttattggtgcggctatatg

SEQ ID NO: 356 - DNA, 225 bp, Artificial Sequence
  BP_DNA_021 Fragment containing sprG1
aggagacgcctaatgattacaattagtaccatgttgcagtttggtttattccttattgcattgataggtctagtaatcaagcttattgaattaagcaataaaaaataaccatcgctaactttggctggtttcgatggttaaatggttattaatttaatctttaatctaaaatagccaccgtctttttaacgggctcattagggtaacatgtttgcgcatgttgcc

SEQ ID NO: 357 - DNA, 267 bp, Artificial Sequence
  Staphylococcus aureus yoeB
atgagcaattacacggttaagattaaaaattcagcgaaatcagatttaaggaaaataaaacattcttatttaaagaagtcatttttagaaattgttgagactttaaaaaatgatccgtataaaataacacaatcttttgaaaaattagagcctaaatatttagagcgatattcaagaagaattaaccatcagcacagggtcgtctataccgtagatgatcgaaataaagaagtattaatactatcggcatggtcacattatgattaa

SEQ ID NO: 358 - DNA, 33 bp, Artificial Sequence
  BP_DNA_023 code_1
cgatcttcgacatcggaccctagaacagaacta

SEQ ID NO: 359 - DNA, 981 bp, Artificial Sequence
  BP_DNA_029 isdB Upstream HA
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaataaatc

SEQ ID NO: 360 - DNA, 412 bp, Artificial Sequence
  BP_DNA_030 PgyrB-sprA1(AS)(long)
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatatataacgaaaataagtatttacttatacaccaatcccctcactatttgcggtagtgaggggatttttattggtgcggctatatg

SEQ ID NO: 361 - DNA, 990 bp, Artificial Sequence
  BP_DNA_031 harA Upstream Homology Arm
tcagttatggacggctttgttgaacatccattctatacagcaactttaaatggtcaaaaatatgtagtgatgaaaacaaaggatgacagttactggaaagatttaattgtagaaggtaaacgtgtcactactgtttctaaagatcctaaaaataattctagaacgttgattttcccatatatacctgacaaagcagtttacaatgcgattgttaaagtcgttgtggcaaacattggttatgaaggtcaatatcatgtcagaattataaatcaggatatcaatacaaaagatgatgatacatcacaaaataacacgagtgaaccgctaaatgtacaaacaggacaagaaggtaaggttgctgatacagatgtagctgaaaatagcagcactgcaacaaatcctaaagatgcgtctgataaagcagatgtgatagaaccagagtctgacgtggttaaagatgctgataataatattgataaagatgtgcaacatgatgttgatcatttatccgatatgtcggataataatcacttcgataagtatgatttaaaagaaatggatactcaaattgccaaagatactgatagaaatgtggataatagcgttggtatgtcatcgaatgtcgatactgataaagactctaataaaaataaagacaaagtcatacagcttgctcatattgccgataaaaataatcatactggaaaagcagcaaagcttgacgtagtgaaacaaaattataataatacagacaaagttactgacaaaaaaacaactgaacatctgccgagtgatattcataaaactgtagataaaacagtgaaaacaaaagaaaaagccggcacaccatcgaaagaaaacaaacttagtcaatctaaaatgctaccaaaaactggagaaacaacttcaagccaatcatggtggggcttatatgcgttattaggtatgttagctttattcattcctaaattcagaaaagaatctaaataa

SEQ ID NO: 362 - DNA, 116 bp, Artificial Sequence
  BP_DNA_032 sprA1.55delT.S21LfsX4 plus control arm
cgcagagaggaggtgtataaggtgatgcttattttcgttcacatcatagcaccagtcatcagtggctgtgccattgcgttttttcttattggctaagtagacgcaatacaaaatag

SEQ ID NO: 363 - DNA, 993 bp, Artificial Sequence
  BP_DNA_033 harA Downstream Homology Arm
ttaactaaatatagcatatgtatggttaactttgtaaacaatgtgaaagcaattaatttataaactattgattggtttaatggctttcctttagagtaaataaaaagaacagcagtgagaaattttctaattgaaaataatcttactgctgtttttaatatttggatgcattgttgtggttactttaaaaagtgagcatcaattaacgcttttttcgatttaacaaatgtgatttaatatcatattttaatgcgtcgttgtattctttttcagtgatttgatcttcgattaacatacgctttaatacataatgttgtctttgaatactatatttcaaatctttatccgattttaacgttccatctttttcgtagggtgtatagccataagggctttgcaacaaaccgataaggtatgcagattgtgcaattgataaatcttttggtggaataccaaacaaactatatgaagcggatgcaattccggaaatattagcgccattataatctctaccgaagggaactatatttaaatatgtatatataatttcatcttttgagagtaggtgttctaatctaattgctaggcgaagttcatttgcttttctactatatgttttttcgttggtaagaacttgatttttaacaagttgttgtgtaattgtgctaccacctgaactttgatcagtattaaaaatatcttgtatcattgctcttaaaatcgcctttggtaagatgccatcatgtttataaaataaagtgtcttcagatgatgttaatgctttaatgacatttggacttgatgttttagggcctataatgagtgagttttgagaatggttatactcatataataaatttttgttattatgatctaataattcatcgccaggtatttgtcgaactttttttattaaagcatcatctgataatgagtcggacgttttagttaaatgatgaaaatataaagacatcgcaatcacagcgatagcaa

SEQ ID NO: 364 - DNA, 93 bp, Artificial Sequence; identical to SEQ ID NO: 284
  Staphylococcus aureus sprA1 toxin

SEQ ID NO: 365 - DNA, 398 bp, Artificial Sequence
  BP_DNA_040 PgyrB-sprA1(AS)(short)
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatatataacgaaaataagtatttacttatacaccaatcccctcactatttgcggtagtgaggggatttttatt

SEQ ID NO: 366 - DNA, 991 bp, Artificial Sequence
  BP_DNA_041 PsbnA Upstream Homology Arm
aagcgcttcctcctcaaatttaaaattctataatattgtgtgttacctaattgataatgattctcactatcaagtaattaggattatattttttatgcatttatatgtcaaataattataagttgcatgtaaatcataaatattttactgacttaggaaaaaatttaattcatactaaatcgtgataatgattctcattgtcatacatcacgaaggaggctaattagtcaatgaataaagtaattaaaatgcttgttgttacgcttgctttcctacttgttttagcaggatgtagtgggaattcaaataaacaatcatctgataacaaagataaggaaacaacttcaattaaacatgcaatgggtacaactgaaattaaagggaaaccaaagcgtgttgttacgctatatcaaggtgccactgacgtcgctgtatctttaggtgttaaacctgtaggtgctgtagaatcatggacacaaaaaccgaaattcgaatacataaaaaatgatttaaaagatactaagattgtaggtcaagaacctgcacctaacttagaggaaatctctaaattaaaaccggacttaattgtcgcgtcaaaagttagaaatgaaaaagtttacgatcaattatctaaaatcgcaccaacagtttctactgatacagttttcaaattcaaagatacaactaagttaatggggaaagctttagggaaagaaaaagaagctgaagatttacttaaaaagtacgatgataaagtagctgcattccaaaaagatgcaaaagcaaagtataaagatgcatggccattgaaagcttcagttgttaacttccgtgctgatcatacaagaatttatgctggtggatatgctggtgaaatcttaaatgatttaggattcaaacgtaataaagacttacaaaaacaagttgataatggtaaagatattatccaacttacatctaaagaaagcattccattaatgaacgctgatca

SEQ ID NO: 367 - DNA, 69 bp, Artificial Sequence
  BP_DNA_043 sprA1(AS)(short)
ataacgaaaataagtatttacttatacaccaatcccctcactatttgcggtagtgaggggatttttatt

SEQ ID NO: 368 - DNA, 94 bp, Artificial Sequence
  BP_DNA_045 delta sprA1 (p253)
atataatagtagagtcgcctatctctcaggcgtcaatttagacgcagagaggaggtgtataaggtgatgcttattttcgttcacatcatagcac

SEQ ID NO: 369 - DNA, 300 bp, Artificial Sequence
  BP_DNA_056 PsbnA (insert in p242)
tcccactacatcctgctaaaacaagtaggaaagcaagcgtaacaacaagcattttaattactttattcattgactaattagcctccttcgtgatgtatgacaatgagaatcattatcacgatttagtatgaattaaattttttcctaagtcaataaaatatttatgatttacatgcaacttataattatttgacatataaatgcataaaaaatataatcctaattacttgatagtgagaatcattatcaattaggtaacacacaatattatagaattttaaatttgaggaggaagcgctt

SEQ ID NO: 370 - DNA, 1247 bp, Artificial Sequence
  BP_DNA_057 Downstream HA (delta PsprA1)
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttatctcaattatactgctaatctttctctttgtaaaatatgcaaggttttaaagagaaacatcaagaactaaaaaaggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 371 - DNA, 333 bp, Artificial Sequence
  BP_DNA_060 PsprA1(AS)
cagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattgag

SEQ ID NO: 372 - DNA, 8793 bp, Artificial Sequence
  BP_DNA_062 pIMAYz Vector
caagcttatcgataccgtcgacctcgagggggggcccggtacccagcttttgttccctttagtgagggttaattatcccattatgctttggcagtttattcttgacatgtagtgagggggctggtataatcacatacggccgataaagcaagcatataatattgcgtttcatctttagaagcgaatttcgccaatattataattatcaaagagaggggtggcaaacggtatttggcattattaggttaaaaaatgtagaaggagagtgaaacccatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataaaggaggatatccatggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataatatgagataatgccgactgtactttttacagtcggttttctaatgtcactaacctgccccgttagttgacatatgatcatcataattctgtctcattatataacatcctccataccttctattatagaatgtattgctattaatcgcaacatcaaaccaaaataaaaacccccttcgactttcgtcagggggcttttatttattcaataatccctcctctcaataaatctattgttgtacttaattcaacttccatttctctgtatctttcaatacgctcttttaagtccttaatttctttttttaattcctcattttcagcaaataactctttttctttgtttgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaaaagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatatgaacctttgatataatcaagtatctcaacatgagcaactgaactattccccaattttcgcttaatcttgttcctaacgctttctattgttacaggatttcgtgcaatatatataacgtgatagtgtggttttttatagtgctttccattttgtataatattactattattccatgtatctttatcttttttttcgtccatatcgtgtaaaggactgacagccatagatacgcccaaactctctaatttttctttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgcatgctgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacactctatcattgatagagttatttgtcaaactagttttttatttggatcgcgtcgagttcatgaaaaactaaaaaaaatattgacactctatcattgatagagtataattaaaataagcttgatgggaatatgataatagaaataccattaccaacaccgaactgagtgatttgatcaccaagccatattaagaaagcagttcctgctgtcaaaactagtgctattaataaataactcataattgactgattgataatcagcgcacctttgagataattattaaattggaatgccatacctatagattggataaatgctaaagaaattgctaaataacgagtaacgttatttaactttcttctacctacttcaccttgttttgcccattctgagaatttagggacaatatccatttgtaataattgcattacgattgatgcagtgatgtagggtacaatacccattgcaaaaatagaaaatcgtttcaaggctccgccaccaaaagtatttaataactcagtggcaccttgagaaccttggggattatcaaaagctgcaggatttactcctggagctggtatataagtccctattttaaaaattactaacattgctagtgtgaagaaaatcttgttacgaacctcttttgttctaaagaagttcacaagggtttgaatcattagatcaagatctcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggacgtcgtaatacgactcactatagggcgaattggagctccaccgcggtggcggccgctctagaactagtggatccc

SEQ ID NO: 373 - DNA, 124 bp, Artificial Sequence
  BP_DNA_065 BP_115 insertion sequence
tgaatagcgcagagaggaggtgtataaggtgatgcttattttcgttcacatcatagcaccagtcatcagtggctgtgccattgcgtttttttcttattggctaagtagacgcaatacaaaatag

SEQ ID NO: 374 - DNA, 150 bp, Artificial Sequence
  BP_DNA_067 hokB gene from E. coli (K12)
atgaagcacaaccctctggtggtgtgtctgctcattatctgcattacgattctgacattcacactcctgacccgacaaacgctctacgaactgcggttccgggacggtgataaggaggttgctgcgctcatggcctgcacgtccaggtaa

SEQ ID NO: 375 - DNA, 156 bp, Artificial Sequence; identical to SEQ ID NO: 127
  BP_DNA_068 hokD gene from E. coli (K12)

SEQ ID NO: 376 - DNA, 336 bp, Artificial Sequence
  BP_DNA_069 mazF gene from E. coli (K12)
atggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattgggtag

SEQ ID NO: 377 - DNA, 279 bp, Artificial Sequence
  BP_DNA_070 yafQ gene from E. coli (K12)
atgattcaaagggatattgaatactcgggacaatattcaaaggatgtaaaacttgcacaaaagcgtcataaggatatgaataaattgaaatatcttatgacgcttcttatcaataatactttaccgcttccagctgtttataaagaccacccgctgcaaggttcatggaaaggttatcgcgatgctcatgtcgaaccggactggatcctgatttacaaacttaccgataaacttttacgatttgagagaactggaactcacgcggcgctctttgggtaa

SEQ ID NO: 378 - DNA, 288 bp, Artificial Sequence
  BP_DNA_071 relE gene from E. coli (K12)
atggcgtattttctggattttgacgagcgggcactaaaggaatggcgaaagctgggctcgacggtacgtgaacagttgaaaaagaagctggttgaagtacttgagtcaccccggattgaagcaaacaagctccgtggtatgcctgattgttacaagattaagctccggtcttcaggctatcgccttgtataccaggttatagacgagaaagttgtcgttttcgtgatttctgttgggaaaagagaacgctcggaagtatatagcgaggcggtcaaacgcattctctga

SEQ ID NO: 379 - DNA, 1027 bp, Artificial Sequence
  BP_DNA_075 tetR_Pxyl-tet
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtg

SEQ ID NO: 380 - DNA, 1084 bp, Artificial Sequence
  BP_DNA_076 kanR PCR fragment
gtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttg

SEQ ID NO: 381 - DNA, 717 bp, Artificial Sequence
  BP_DNA_077 GFPmut2
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 382 - DNA, 6183 bp, Artificial Sequence
  BP_DNA_086 linearized pMBsacB
ggagaggcggtttgcgtattggtcgggtaccgagctcgaattgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaataatagtaatattatacaaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattagcttgttgtaactgaaaaaggaaaattattgtgccaggcagttgaaagtcagcaccttttaacgagtgctgaaatgacggctaaatgggaaacgtatttaaaaaaaatcggtaaaagagaaggcaatcaagagaactttattacgaatatcaaaaaattcattgttcatttactggaagctgtacctaacgatatagaaaaactaaatttttctgattaccaggaacagaaagaaaaagaagcagaaaaaagtattgtaggaaaatgtcctaagtgtggcaacaatattgtattaaaaaaatcgttttatggttgttcaaattatcctgaatgtaagtttactttagctgaacattttagaaagaaaaaactcaccaaaacaaatgtaaaagaattactagagggaaaagaaaccctggtaaaaggaatcaaaacgaaagatagaaagtcctacaatgccgttgtaaaaatcggagaaaagggatatattgattttatatctttctcaaaataaacataaaagccctttaaagagggcttttatatattaatcacaaatcacttatcacaaatcacaagtgatttgtgattgttgatgataaaataagaataagaagaaatagaaagaagtgagtgattgtgggaaatttaggcgcacaaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccttaactaaaagtagtgaatttttgatttttggtgtgtgtgtcttgttgttagtatttgctagtcaagtgattaaatagaattcgaaaagccctgacaacccttgttcctaaaaaggaataagcgttcggtcagtaaataatagaaataaaaaatcagacctaagactgatgacaaaaagagaaaattttgataaaatagtcttagaattaaattaaaaagggaggccaaatataatgaaaaatatgaatgacaatgatgttgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataacgcatgcctagcgcctacggggaatttgtatcgataaggggtacaaattcccactaagcgctcggtggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatg

SEQ ID NO: 383 - DNA, 699 bp, Artificial Sequence
  BP_DNA_087 mKATE2 (Staph aureus codon optimizaed)
atggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatga

SEQ ID NO: 384 - DNA, 1938 bp, Artificial Sequence
  Staphylococcus aureus isdB
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 385 - DNA, 1968 bp, Artificial Sequence
  Staphylococcus aureus isdB.1943delC.STOP645NfsX11
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaataataaatccgcagagaggaggtgtataaggtga

SEQ ID NO: 386 - DNA, 72 bp, Artificial Sequence
  Staphylococcus aureus sprA1.55delT.S21LfsX4
atgcttattttcgttcacatcatagcaccagtcatcagtggctgtgccattgcgttttttcttattggctaa

SEQ ID NO: 387 - DNA, 738 bp, Artificial Sequence; identical to SEQ ID NO: 317
  BP_DNA_091 lysostaphin

SEQ ID NO: 388 - DNA, 991 bp, Artificial Sequence
  BP_DNA_092 PsbnA Upstream Homology Arm
aagcgcttcctcctcaaatttaaaattctataatattgtgtgttacctaattgataatgattctcactatcaagtaattaggattatattttttatgcatttatatgtcaaataattataagttgcatgtaaatcataaatattttattgacttaggaaaaaatttaattcatactaaatcgtgataatgattctcattgtcatacatcacgaaggaggctaattagtcaatgaataaagtaattaaaatgcttgttgttacgcttgctttcctacttgttttagcaggatgtagtgggaattcaaataaacaatcatctgataacaaagataaggaaacaacttcaattaaacatgcaatgggtacaactgaaattaaagggaaaccaaagcgtgttgttacgctatatcaaggtgccactgacgtcgctgtatctttaggtgttaaacctgtaggtgctgtagaatcatggacacaaaaaccgaaattcgaatacataaaaaatgatttaaaagatactaagattgtaggtcaagaacctgcacctaacttagaggaaatctctaaattaaaaccggacttaattgtcgcgtcaaaagttagaaatgaaaaagtttacgatcaattatctaaaatcgcaccaacagtttctactgatacagttttcaaattcaaagatacaactaagttaatggggaaagctttagggaaagaaaaagaagctgaagatttacttaaaaagtacgatgataaagtagctgcattccaaaaagatgcaaaagcaaagtataaagatgcatggccattgaaagcttcagttgttaacttccgtgctgatcatacaagaatttatgctggtggatatgctggtgaaatcttaaatgatttaggattcaaacgtaataaagacttacaaaaacaagttgataatggtaaagatattatccaacttacatctaaagaaagcattccattaatgaacgctgatca

SEQ ID NO: 389 - DNA, 1867 bp, Artificial Sequence
  BP_DNA_093 tetR_Ptet-GFPmut2 Fragment
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagtgacatatagccgcaccaataaaaattgataatagctgagcccgggcactggccgtcgttttacaacgtcgtgactgggaaaaccctggcgttacccaacttaatcgccttgcagcacatcc

SEQ ID NO: 390 - DNA, 723 bp, Artificial Sequence
  BP_DNA_094 mKATE2 with control arm
cgcagagaggaggtgtataaggtgatggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatga

SEQ ID NO: 391 - DNA, 6511 bp, Artificial Sequence
  BP_DNA_095 pRAB11 Linearized Plasmid Backbone (p151)
gtgacatatagccgcaccaataaaaattgataatagctgagcccgggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcggacagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggtttttaggcataaaactatatgatttacccctaaatctttaaaatgccccttaaaattcaaaataaaggcatttaaaatttaaatatttcttgtgataaagtttgttaaaaaggagtggttttatgactgttatgtggttatcgattataggtatgtggttttgtattggaatggcattttttgctatcaaggttattaaaaataaaaattagaccacgcatttatgccgagaaaatttattgtgcgttgagaagaacccttaactaaacttgcagacgaatgtcggcatagcgtgagctattaagccgaccattcgacaagttttgggattgttaagggttccgaggctcaacgtcaataaagcaattggaataaagaagcgaaaaaggagaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccggcgaggctagttacccttaagttattggtatgactggttttaagcgcaaaaaaagttgctttttcgtacctattaatgtatcgttttaaatgaatagtaaaaaacatacatagaaaggggaaaaagcaactttttttattgtcatagtttgtgaaaactaagttgtttttatgtgttataacatggaaaagtatactgagaaaaaacaaagaaatcaagtatttcagaaatttattaaacgtcatattggagagaatcaaatggatttagttgaagattgcaatacatttctgtcttttgtagctgataaaactttagaaaaacagaaattatataaagctaattcttgtaaaaatcgattttgtcctgtctgtgcttggagaaaagctagaaaagatgcattgggtttatctttgatgatgcaatatattaagcagcaagagaaaaaggagtttatctttttaactttgactacacctaatgtaatgagtgatgaattagaaaatgaaataaaacgttataataattcttttagaaaacttataaagagaaaaaaagtaggtagtgttataaagggatatgttcgtaagttagagattacatataataaaaaaagagatgattataatcctcattttcatgtgttaattgcagtaaataaatcgtatttcacagataaaagatattatattagccaacaagaatggttagatttatggcgtgatgtaacgggcatttcagaaataacacaagttcaagttcaaaaaataagacaaaataataataaagaattatatgaaatggctaagtattctggtaaagatagtgattatttaataaatcaaaaagtctttgatgcattttataaatcacttaaaggtaaacaggtattagtttattcaggattatttaaagaggctaaaaagaaattaaaaaatggggatttagattacttaaaagaaattgatccaaccgaatatatctatcaaattttttatatttggaaacaaaaagagtatttagctagtgaactttatgacttaacagaacaagaaaaaagagaaattaatcacaaaatgatagacgaaatcgaggaagaacaataacaaaatataagtgctaacagctgacctcccgataacaccatgtagttattgggaggtcagctgttgaattatgcacgagtattttaaaagttattgtgatgacgacgataaacgattatcaaaagtataatgttaaaatgctttattatactaacgttatataaacattatactttcgttatacaaattttaaccctgttaggaactataaaaaatcatgaaaattttaatttgcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtg

SEQ ID NO: 392 - DNA, 60 bp, Artificial Sequence
  Staphylococcus aureus sprA1(AS)
ataacgaaaataagtatttacttatacaccaatcccctcactatttgcggtagtgagggg

SEQ ID NO: 393 - DNA, 58 bp, Artificial Sequence
  Staphylococcus aureus sprA2(AS)
cataataaattgaacatctaaatacaccaaatcccctcactactgccatagtgagggg

SEQ ID NO: 394 - DNA, 143 bp, Artificial Sequence
  Staphylococcus aureus sprF
tatatagaaaaagggcaacatgcgcaaacatgttaccctaatgagcccgttaaaaagacggtggctattttagattaaagattaaattaataaccatttaaccatcgaaaccagccaaagttagcgatggttattttttattg

SEQ ID NO: 395 - DNA, 252 bp, Artificial Sequence
  BP_DNA_107  yefm
atgcgtacaattagctacagcgaagcgcgtcagaatttgtcggcaacaatgatgaaagccgttgaagatcatgccccgatccttattactcgtcagaatggagaggcttgtgttctgatgtcactcgaagaatacaactcgctggaagagacggcttatctactgcgctcccccgctaacgcccggagattgatggactcaatcgatagcctgaaatcaggcaaaggaacggaaaaggacatcattgagtga

SEQ ID NO: 396 - DNA, 744 bp, Artificial Sequence
  Serratia marcescens smaI
atgagcagggatgaccaactctttacactttggggaaagcttaacgatcgtcagaaggataattttctaaaatggatgaaagcttttgatgtagagaaaacttaccaaaaaacaagtggggatattttcaatgatgattttttcgatatatttggtgatagattaattactcatcatttcagtagcacgcaagctttaacaaaaactttattcgaacatgcttttaatgactccttaaatgaatctggagttatatcctctcttgcggaaagtagaacaaaccctgggcatgacataacaatcgatagcataaaggttgctttaaaaacagaagcagctaaaaatattagcaaatcatatattcatgtaagtaagtggatggagttaggcaagggggagtggattctagaattattattagaacggtttttagagcatctagagaattatgaacgtattttcacactcagatattttaaaatatccgagtataaatttagctaccagcttgtagaaatacccaagagtcttttgttggaagcaaaaaatgcgaaattagaaataatgtcgggaagcaaacaaagccctaagcccggctatggatatgtgttagatgaaaatgaaaataagaagttttctctatactttgatggtggtgccgagagaaaacttcaaataaaacatttaaatttagaacattgcattgttcatggagtttgggattttattctaccgccgccttaa

SEQ ID NO: 397 - DNA, 177 bp, Artificial Sequence
  BP_DNA_109 delta sprA1-sprA1(AS)
agataacgaaaataagtatttacttatacaccaatcccctcactatttgcggtagtgaggggatttttattggtgcggctatatgtcacctattttgtattgcgtctacttagccaataagaaaaaaacgcaatggcacagccactgatgactggtgctatgatgtgaacgaaaata

SEQ ID NO: 398 - DNA, 555 bp, Artificial Sequence
  BP_DNA_110 Upstream HA for delta sprA1-sprA1(AS)
agcatcaccttatacacctcctctctgcgtctaaattgacgcctgagagataggcgactctactattatatcatctaattggacaaattctatgagagtagattttgttaatttaagaaagaagaacatttagttcagagtttaagttataaatggttagattatatgaaaatggtaagccaagaaatgaaattatacgcgagtatgatttaacacctttgacgttaggaaaatggataaagtaacatcaaaacaagggtacattcaatcaccaagataacttaacagatgaagaaaaagagctgattatggggcaaaaatagaagtcattcaaaagaatgcacatcaatattcagtaccagcaatgtgtaaagtcctaaaaataccaagaagtacctattatgattctataaaaagaaaagataataaaatcactaaagatgattcaaacgtagaacgtgccgccataaatatttttaattctaatagaaaagtctttagtacaagacgaattaaaaatcatttaaatgacaagggtctcactgtatctggaca

SEQ ID NO: 399 - DNA, 108 bp, Artificial Sequence
  Staphylococcus aureus sprG2
gtgatatctattgcaaacgcattacatttaatgttaagtttcggtatgtttatcgtcactttcattggtatagtagtagcaataataaatttaagcaataaaaaataa

SEQ ID NO: 400 - DNA, 116 bp, Artificial Sequence
  Staphylococcus aureus sprF2
gtaaaaagacgacatgcaggaacatgtcgcctaatgagcccgttaaaaagacggtgactaaatgagattttctttaaccatcattctatgtcaaagttttgaaatgatggttattt

SEQ ID NO: 401 - DNA, 81 bp, Artificial Sequence
  Staphylococcus aureus sprG3
atgtctgattttgaaatgctgatggttgtattaacaatcattggtttagtattgattagtactcaagaccataaaaaataa

SEQ ID NO: 402 - DNA, 164 bp, Artificial Sequence
  Staphylococcus aureus sprF3
gtagtaagtagaagcaaaagatgaaaatctttaactcttgaaacacaaaaagggcaacactcggaaacatgttaccctaatgagcccgttaaaaagacggtgaccttatattttatttaaaaatagccttcaaaatgccggtcaaagcgaatagaaggttattt

SEQ ID NO: 403 - DNA, 531 bp, Artificial Sequence
  BP_DNA_115 Downstream HA for delta sprA1-sprA1(AS)
aaattactttcagactctagtgaacaaaagttctactataagcttattctttggcaatatttgaaagataaacatggattaacctgtgcatattctacatttagagcttatatccttaaacatgatgaatttaatcgttattttatgaaaggttatcaacggttgtcgcctaaaggcaaaacgagattcgagacaaaagccagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattg

SEQ ID NO: 404 - DNA, 1849 bp, Artificial Sequence
  BP_DNA_116 tetR_Ptet-mKATE2 fragment
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtgatggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatgagtgacatatagccgcaccaataaaaattgataatagctgagcccgggcactggccgtcgttttacaacgtcgtgactgggaaaaccctggcgttacccaacttaatcgccttgcagcacatcc

SEQ ID NO: 405 - DNA, 741 bp, Artificial Sequence
  BP_DNA_117 GFPmut2 with control arm
cgcagagaggaggtgtataaggtgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 406 - DNA, 1063 bp, Artificial Sequence
  BP_DNA_118 Upstream HA (?sprA1-sprA1(AS) in p197)
catctaattggacaaattctatgagagtagattttgttaatttaagaaagaagaacatttagttcagagtttaagttataaatggttagattatatgaaaatggtaagccaagaaatgaaattatacgcgagtatgatttaacacctttgacgttaggaaaatggataaagtaacatcaaaacaagggtacattcaatcaccaagataacttaacagatgaagaaaaagagctgattatggggcaaaaatagaagtcattcaaaagaatgcacatcaatattcagtaccagcaatgtgtaaagtcctaaaaataccaagaagtacctattatgattctataaaaagaaaagataataaaatcactaaagatgattcaaacgtagaacgtgccgccataaatatttttaattctaatagaaaagtctttagtacaagacgaattaaaaatcatttaaatgacaagggtctcactgtatctggacaaaagataggtcgattatgaaaaaatctagtttctgtttatatgaaagctaaatacaaaaatcatcaaatagaaactaatgaaaaacgaattaaaaatcatttgaatcgcgcttttaatagagaacaaccaatggagacattagtaagtgatttgacatatgtaaaagtcggaggaacatggcattacatatgtttatttatagatctatttaatagagaaattgttggttacagtgcaggtaaaaataaggacgccaatttagtatcaaaagcaatcagcagaataaatcataatcttgaacaaatcaaactattccacactgatagagacaaagaatttgataatcatttgatagatgaagtcctagaaacatttaaaataaaacgttcattaagtaccaaaggttgtccttatgataacgcagttgcggaagcaacgatgaaagcaatgaaaacagaatttgtaaaacaaatgcaatttgaaaacttagaacagttagaaacagaattatttgattatgtaaattggtacaacaattttagaccacattcttcattacagtatttgacgccagtggc

SEQ ID NO: 407 - DNA, 970 bp, Artificial Sequence
  BP_DNA_119 Downstream HA (delta sprA1-sprA1(AS) in p197)
ggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 408 - DNA, 2040 bp, Artificial Sequence
  BP_DNA_120 D7VX69 prolyl endopeptidase (Chryseobacterium teaenense)
atgatgtatcccaaagcattaaaaggaaagcaaaccgataattattttggaactgctgttacagatccgttcagagatcttgaaaatgattctgaagccaccaaaaagtgggtcgacgaagaagtgaaacacagtcaggattatcttgcaaaaatccctttcagagaagaaatcaggaaacagctcaccgatatctggaactacgaaaaaatttcggctccttttaaagaaggtgattttacttattattataaaaacaacgggctacaggcacaatctgtactttacagaaccaacaataaaacgaaagaaacagaagtatttttagatccgaataaattttctgaaaaaggcaccacttcactttctcaattgtcttttaacaaaaaaggaaatctcgccgcttattctatttcagaaggaggaagtgactggaacaagatcattatcatagacgctttatctaaaaaacagatcgatgaaacgctggtagatgtaaagttcagcggaatttcctggcagggtgatgaaggtttctattattcaagctacgacaaaccgaaagagggaaccgtgctctccggcatgaccgataagcataaagtctattttcataagttaggaacaaagcagtccgaagatcaattgatttttggaggagataaaacaccgagaagatatttgggagcaggagtgtctgaagatcagagatatcttattatttctgctgcgaatgccaccaacggaaatgaattatacataaaagaccttaaaaacggaggagattttgtgcagattaataaaggttttgatatcaatgccgatatagtcgatacacaaggagacgatctgtatatctttaccgataaggatgccccgaatatgcgtctcgtaaaaacaaccattaaaaatcctgctccagagacttggaaagatgtaattccggaaacggaaaatgttttgggaatcacaacaggcggaggatatttctttgctacctatatggtggatgcgattgatcaggtaaaacaatacgacagagcaggaaaaatgatccgtgaaattacgcttcccggaaaaggaaatgtttctggttttggaggaaaggaaaacgagaaagaattgtatttttcattcaccaattatattacaccgggaacaacgtataaattcaatgcagattccggaaaatctgaggtttaccagaagccgaaggtgaaatttaatcctgaagattatgtctctgaacaagtattttacacctcaaaagacggtacaaaagttccgatgatgattaactataaaaaaggaactaagctcgacggtaaaaatcctacgattttatattcttacggaggttttaatatcagtttgcagccggccttctctgtagtcaatgccatctggatggaaaacggtggtatttatgccgttccgaacatccgtggaggtggtgaatatggaaaaaaatggcatgatgccggaacaaaaatgaataagaaaaacgtattcaacgatttcatcgctgccggagaatatttgcagcagaaaggctacacttccaaacagtttatggctctttcgggaagatcaaatggaggactgctggtgggtgcaacaatgacgatgcgtcccgatctggcaagagtcgctttcccgggagttggcgtgttggatatgctgaggtataataaattcacagccggtgcaggttggtcgtacgattacggaacctctgaagatagcaaagaaatgtttgaatatttaaaatcatattccccggttcataatgtaaaagccggaacatgctatccttccacaatgattatcaccagcgatcatgatgacagggtggttcctgcgcattctttcaaattcggagctgagcttcaggaaaagcaggcatgcgatcatccgattcttttaaggattgaaaaaaatgcaggtcacggagcaggcagatctacagatcaggtgatcggagaaaatgcagacttgatttctttcgctttatttgaaatggggattaaaaatttaaag

SEQ ID NO: 409 - DNA, 1197 bp, Artificial Sequence
  BP_DNA_121 Actinoallomurus sp. strain DSM 24988 ENDOPEP-40 gene, complete cds
atgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttctga

SEQ ID NO: 410 - protein, 655 aa, Artificial Sequence
  Staphylococcus aureus isdB
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNNKSAERRCIR

SEQ ID NO: 411 - protein, 30 aa, Artificial Sequence; identical to SEQ ID NO: 104
  Staphylococcus aureus sprA1

SEQ ID NO: 412 - protein, 35 aa, Artificial Sequence; identical to SEQ ID NO: 305
  Staphylococcus aureus sprA2

SEQ ID NO: 413 - protein, 645 aa, Artificial Sequence
  Staphylococcus aureus isdB.1943delC.STOP645NfsX11
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 414 - protein, 49 aa, Artificial Sequence
  BP_AA_005 hokB (E. coli, K12)
MKHNPLVVCLLIICITILTFTLLTRQTLYELRFRDGDKEVAALMACTSR

SEQ ID NO: 415 - protein, 51 aa, Artificial Sequence
  BP_AA_006 hokD (relF) (E. coli, K12)
MKQQKAMLIALIVICLTVIVTALVTRKDLCEVRIRTGQTEVAVFTAYEPEE

SEQ ID NO: 416 - protein, 111 aa, Artificial Sequence
  BP_AA_007 mazF (E. coli, K12)
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 417 - protein, 92 aa, Artificial Sequence
  BP_AA_008 yafQ (E. coli, K12)
MIQRDIEYSGQYSKDVKLAQKRHKDMNKLKYLMTLLINNTLPLPAVYKDHPLQGSWKGYRDAHVEPDWILIYKLTDKLLRFERTGTHAALFG

SEQ ID NO: 418 - protein, 95 aa, Artificial Sequence
  BP_AA_009 relE (E. coli, K12)
MAYFLDFDERALKEWRKLGSTVREQLKKKLVEVLESPRIEANKLRGMPDCYKIKLRSSGYRLVYQVIDEKVVVFVISVGKRERSEVYSEAVKRIL

SEQ ID NO: 419 - protein, 207 aa, Artificial Sequence
  BP_AA_010 tetR (artificial sequence, synthetic construct)
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 420 - protein, 264 aa, Artificial Sequence [sequence omitted]

SEQ ID NO: 421 - protein, 238 aa, Artificial Sequence
  BP_AA_012 GFP (green fluorescent protein)
MSKGEELFTGVVPILVELDGDVNGHKFSVSGEGEGDATYGKLTLKFICTTGKLPVPWPTLVTTFAYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 422 - protein, 232 aa, Artificial Sequence
  BP_AA_013 mKATE2 (RFP, red fluorescent protein)
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 423 - protein, 23 aa, Artificial Sequence
  BP_AA_014 sprA1.55delT.S21LfsX4 (truncated version of sprA1 in Staph aureus BP_001)
MLIFVHIIAPVISGCAIAFFLIG

SEQ ID NO: 424 - protein, 88 aa, Artificial Sequence; identical to SEQ ID NO: 316
  Staphylococcus aureus yoeb

SEQ ID NO: 425 - protein, 245 aa, Artificial Sequence; identical to SEQ ID NO: 318
  BP_AA_016 lysostaphin (artificial sequence)

SEQ ID NO: 426 - protein, 83 aa, Artificial Sequence
  BP_AA_017 yefM (E.coli)
MRTISYSEARQNLSATMMKAVEDHAPILITRQNGEACVLMSLEEYNSLEETAYLLRSPANARRLMDSIDSLKSGKGTEKDIIE

SEQ ID NO: 427 - protein, 247 aa, Artificial Sequence
  Serratia marcescens smaI
MSRDDQLFTLWGKLNDRQKDNFLKWMKAFDVEKTYQKTSGDIFNDDFFDIFGDRLITHHFSSTQALTKTLFEHAFNDSLNESGVISSLAESRTNPGHDITIDSIKVALKTEAAKNISKSYIHVSKWMELGKGEWILELLLERFLEHLENYERIFTLRYFKISEYKFSYQLVEIPKSLLLEAKNAKLEIMSGSKQSPKPGYGYVLDENENKKFSLYFDGGAERKLQIKHLNLEHCIVHGVWDFILPPP

SEQ ID NO: 428 - protein, 35 aa, Artificial Sequence
  Staphylococcus aureus sprG2
VISIANALHLMLSFGMFIVTFIGIVVAIINLSNKK

SEQ ID NO: 429 - protein, 26 aa, Artificial Sequence
  Staphylococcus aureus sprG3
MSDFEMLMVVLTIIGLVLISTQDHKK

SEQ ID NO: 430 - protein, 31 aa, Artificial Sequence
  Staphylococcus aureus sprG1(short)
MITISTMLQFGLFLIALIGLVIKLIELSNKK

SEQ ID NO: 431 - protein, 680 aa, Artificial Sequence
  BP_AA_022 D7VX69 prolyl endopeptidase (Chryseobacterium taeanense)
MMYPKALKGKQTDNYFGTAVTDPFRDLENDSEATKKWVDEEVKHSQDYLAKIPFREEIRKQLTDIWNYEKISAPFKEGDFTYYYKNNGLQAQSVLYRTNNKTKETEVFLDPNKFSEKGTTSLSQLSFNKKGNLAAYSISEGGSDWNKIIIIDALSKKQIDETLVDVKFSGISWQGDEGFYYSSYDKPKEGTVLSGMTDKHKVYFHKLGTKQSEDQLIFGGDKTPRRYLGAGVSEDQRYLIISAANATNGNELYIKDLKNGGDFVQINKGFDINADIVDTQGDDLYIFTDKDAPNMRLVKTTIKNPAPETWKDVIPETENVLGITTGGGYFFATYMVDAIDQVKQYDRAGKMIREITLPGKGNVSGFGGKENEKELYFSFTNYITPGTTYKFNADSGKSEVYQKPKVKFNPEDYVSEQVFYTSKDGTKVPMMINYKKGTKLDGKNPTILYSYGGFNISLQPAFSVVNAIWMENGGIYAVPNIRGGGEYGKKWHDAGTKMNKKNVFNDFIAAGEYLQQKGYTSKQFMALSGRSNGGLLVGATMTMRPDLARVAFPGVGVLDMLRYNKFTAGAGWSYDYGTSEDSKEMFEYLKSYSPVHNVKAGTCYPSTMIITSDHDDRVVPAHSFKFGAELQEKQACDHPILLRIEKNAGHGAGRSTDQVIGENADLISFALFEMGIKNLK

SEQ ID NO: 432 - protein, 398 aa, Artificial Sequence
  BP_AA_023 Actinoallomurus sp. strain DSM 24988 ENDOPEP-40 gene, complete cds
MSRRVTGTILGGLILAMVPFLSTAANAAPQAAPASVSHPFHHSCATVKPGRASCNALVRSDIAQSAATLAHQAAAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAF

SEQ ID NO: 433 - protein, 1029 aa, Artificial Sequence
  BP_AA_024 E. coli lacZ gene EC3.2.1.23
MTMITPSFPGNSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 434 - DNA, 108 bp, Artificial Sequence
  BP_DNA_125 sprG2 (gDNA.1G>A, 4A>C)
atgctatctattgcaaacgcattacatttaatgttaagtttcggtatgtttatcgtcactttcattggtatagtagtagcaataataaatttaagcaataaaaaataa

SEQ ID NO: 435 - protein, 35 aa, Artificial Sequence
  BP_AA_025 sprG2 (protein.V1M, I2L)
MLSIANALHLMLSFGMFIVTFIGIVVAIINLSNKK

SEQ ID NO: 436 - DNA, 109 bp, Artificial Sequence
  Synthetic construct
ttaagtagcatcgttgcattcgtattacctagaaaacgtaaaaactaataaatccgcagagaggaggtgtataaggtgatgcttattttcgttcacatcatagcaccag

SEQ ID NO: 437 - protein, 15 aa, Artificial Sequence
  Synthetic construct
LSSIVAFVLPRKRKN

SEQ ID NO: 438 - protein, 10 aa, Artificial Sequence
  Synthetic construct
MLIFVHIIAP

SEQ ID NO: 439 - DNA, 108 bp, Artificial Sequence
  Synthetic construct
ttaagtagcatcgttgcattcgtattacctagaaaacgtaaaaataataaatccgcagagaggaggtgtataaggtgatgcttattttcgttcacatcatagcaccag

SEQ ID NO: 440 - protein, 25 aa, Artificial Sequence
  Synthetic construct
LSSIVAFVLPRKRKNNKSAERRCIR

SEQ ID NO: 441 - DNA, 110 bp, Artificial Sequence
  Synthetic construct
ttaagtagcatcgttgcattcgtattacctagaaaacgtaaaaactaattgatagcgcagagaggaggtgtataaggtgatgcttattttcgttcacatcatagcaccag

SEQ ID NO: 442 - DNA, 149 bp, Artificial Sequence
  Synthetic construct
cgcagagaggaggtgtataaggtgatgcttattttcgttcacatcatagcaccagtcatcagtggctgtgccattgcgtttttttcttattggctaagtagacgcaatacaaaataggtctttatatttaattaaattaacaaatttta

SEQ ID NO: 443 - protein, 7 aa, Artificial Sequence
  Synthetic construct
AERRCIR

SEQ ID NO: 444 - protein, 51 aa, Artificial Sequence
  Homo sapiens insulin, B-chain
GIVEQCCTSICSLYQLENYCNFVNQHLCGSHLVEALYLVCGERGFFYTPKT

SEQ ID NO: 445 - DNA, 22 bp, Artificial Sequence
  Synthetic construct; DR_606
gaacaacgtaacggcttcatcc

SEQ ID NO: 446 - DNA, 29 bp, Artificial Sequence
  Synthetic construct; DR_607
gttgctcgtgcatttagatgattcttatc

SEQ ID NO: 447 - DNA, 56 bp, Artificial Sequence
  Synthetic construct; BP_948
ccctcgaggtcgacggtatcgataagcttggatgagcaagtgaaatcagctattac

SEQ ID NO: 448 - DNA, 48 bp, Artificial Sequence
  Synthetic construct; BP_949
cacctcctctctgcggatttattagtttttacgttttctaggtaatac

SEQ ID NO: 449 - DNA, 42 bp, Artificial Sequence
  Synthetic construct; BP_950
aaaaactaataaatccgcagagaggaggtgtataaggtgatg

SEQ ID NO: 450 - DNA, 55 bp, Artificial Sequence
  Synthetic construct; BP_951
attaaatataaagacctattttgtattgcgtctacttagccaataagaaaaaaac

SEQ ID NO: 451 - DNA, 54 bp, Artificial Sequence
  Synthetic construct; BP_952
cgcaatacaaaataggtctttatatttaattattaaattaacaaattttaattg

SEQ ID NO: 452 - DNA, 56 bp, Artificial Sequence
  Synthetic construct; BP_953
gtggcggccgctctagaactagtggatcccgtcaattacgcaattaaggaaatatc

SEQ ID NO: 453 - DNA, 56 bp, Artificial Sequence
  Synthetic construct; DR_511
cacctcctctctgcgctattcaattagtttttacgttttctaggtaatacgaatgc

SEQ ID NO: 454 - DNA, 40 bp, Artificial Sequence
  Synthetic construct; DR_512
ctaattgaatagcgcagagaggaggtgtataaggtgatgc

SEQ ID NO: 455 - DNA, 127 bp, Artificial Sequence
  frameshift sprA1 toxin; BP_DNA_063
ataataaatccgcagagaggaggtgtataaggtgatgcttattttcgttcacatcatagcaccagtcatcagtggctgtgccattgcgtttttttcttattggctaagtagacgcaatacaaaatag

SEQ ID NO: 456 - DNA, 25 bp, Artificial Sequence
  Primer; DR_022
caagcttatcgataccgtcgacctc

SEQ ID NO: 457 - DNA, 23 bp, Artificial Sequence
  Primer; DR_023
gggatccactagttctagagcgg

SEQ ID NO: 458 - DNA, 28 bp, Artificial Sequence
  Primer; DR_116
gggacgtcgtaatacgactcactatagg

SEQ ID NO: 459 - DNA, 40 bp, Artificial Sequence
  Primer; DR_117
ccaaagcataatgggataattaaccctcactaaagggaac

SEQ ID NO: 460 - DNA, 40 bp, Artificial Sequence
  Primer; DR_254
atgcttattttcgttcacatcatagcaccagtcatcagtg

SEQ ID NO: 461 - DNA, 60 bp, Artificial Sequence
  Primer; DR_518
gtggcggccgctctagaactagtggatcccgtcaattacgcaattaaggaaatatcaagg

SEQ ID NO: 462 - DNA, 56 bp, Artificial Sequence; identical to SEQ ID NO: 447
  Primer; BP_948

SEQ ID NO: 463 - DNA, 48 bp, Artificial Sequence; identical to SEQ ID NO: 448
  Primer; BP_949

SEQ ID NO: 464 - DNA, 42 bp, Artificial Sequence; identical to SEQ ID NO: 449
  Primer; BP_950

SEQ ID NO: 465 - DNA, 55 bp, Artificial Sequence; identical to SEQ ID NO: 450
  Primer; BP_951

SEQ ID NO: 466 - DNA, 54 bp, Artificial Sequence; identical to SEQ ID NO: 451
  Primer; BP_952

SEQ ID NO: 467 - DNA, 22 bp, Artificial Sequence
  Primer; BP_964
tcaaacttcagcaggttctagc

SEQ ID NO: 468 - DNA, 22 bp, Artificial Sequence
  Primer; BP_965
gtaccaggtatgactgaatgcc

SEQ ID NO: 469 - DNA, 20 bp, Artificial Sequence
  Primer; gyrB; BPC802
ttggtacaggaatcggtggc

SEQ ID NO: 470 - DNA, 20 bp, Artificial Sequence
  Primer; BPC803
tccatccacatcggcatcag

SEQ ID NO: 471 - DNA, 20 bp, Artificial Sequence
  Primer; isdA; BPC114
gcaacagaagctacgaacgc

SEQ ID NO: 472 - DNA, 22 bp, Artificial Sequence
  Primer; BPC115
agagccatctttttgcacttgg

SEQ ID NO: 473 - DNA, 25 bp, Artificial Sequence
  Primer; isdB; BPC116
gcaacaattttatcattatgccagc

SEQ ID NO: 474 - DNA, 22 bp, Artificial Sequence
  Primer; BPC117
tggcaactttttgtcaccttca

SEQ ID NO: 475 - DNA, 21 bp, Artificial Sequence
  Primer; isdI; BPC764
accgaggatacagacgaagtt

SEQ ID NO: 476 - DNA, 21 bp, Artificial Sequence
  Primer; BPC765
tgctgtccatcgtcatcactt

SEQ ID NO: 477 - DNA, 21 bp, Artificial Sequence
  Primer; isdG; BPC120
aaccaatccgtaaaagcttgc

SEQ ID NO: 478 - DNA, 20 bp, Artificial Sequence
  Primer; BPC121
aggctttgatggcatgtttg

SEQ ID NO: 479 - DNA, 21 bp, Artificial Sequence
  Primer; sbnC; BPC768
agggaagggtgtctaagcaac

SEQ ID NO: 480 - DNA, 20 bp, Artificial Sequence
  Primer; BPC769
tcagtccttcttcaacgcga

SEQ ID NO: 481 - DNA, 20 bp, Artificial Sequence
  Primer; sbnE; BPC124
attcgctttagccgcaatgg

SEQ ID NO: 482 - DNA, 20 bp, Artificial Sequence
  Primer; BPC125
gcaacttgtagcgcatcgtc

SEQ ID NO: 483 - DNA, 21 bp, Artificial Sequence
  Primer; lrgA; BPC126
gataccggctggtacgaagag

SEQ ID NO: 484 - DNA, 21 bp, Artificial Sequence
  Primer; BPC127
tggtgctgttaagttaggcga

SEQ ID NO: 485 - DNA, 20 bp, Artificial Sequence
  Primer; lrgB; BPC128
acaaagacaggcacaactgc

SEQ ID NO: 486 - DNA, 20 bp, Artificial Sequence
  Primer; BPC129
ggtgtagcaccagccaaaga

SEQ ID NO: 487 - DNA, 21 bp, Artificial Sequence
  Primer; hlgB; BPC760
tggttggggaccttatggaag

SEQ ID NO: 488 - DNA, 20 bp, Artificial Sequence
  Primer; BPC761
ggcatttggtgttgcgctat

SEQ ID NO: 489 - DNA, 21 bp, Artificial Sequence
  Primer; fhuA; BPC132
cacgttgtctttgaccaccac

SEQ ID NO: 490 - DNA, 21 bp, Artificial Sequence
  Primer; BPC133
tgggcaatggaagttacagga

SEQ ID NO: 491 - DNA, 20 bp, Artificial Sequence
  Primer; fhuB; BPC134
caatacctgctggaacccca

SEQ ID NO: 492 - DNA, 20 bp, Artificial Sequence
  Primer; BPC135
gggtccgcatattgccaaac

SEQ ID NO: 493 - DNA, 21 bp, Artificial Sequence
  Primer; ear; BPC136
ccacttgtcagatctgctcct

SEQ ID NO: 494 - DNA, 24 bp, Artificial Sequence
  Primer; BPC137
ggtttggttacagatggacaaaca

SEQ ID NO: 495 - DNA, 19 bp, Artificial Sequence
  Primer; fnb; BPC772
cgcagtgagcgaccataca

SEQ ID NO: 496 - DNA, 20 bp, Artificial Sequence
  Primer; BPC773
ttggtccttgtgcttgacca

SEQ ID NO: 497 - DNA, 20 bp, Artificial Sequence
  Primer; hlb; BPC140
ctacgccaccatcttcagca

SEQ ID NO: 498 - DNA, 20 bp, Artificial Sequence
  Primer; BPC141
acacctgtactcggtcgttc

SEQ ID NO: 499 - DNA, 22 bp, Artificial Sequence
  Primer; splF; BPC142
tgcaattattcagcctggtagc

SEQ ID NO: 500 - DNA, 22 bp, Artificial Sequence
  Primer; BPC143
cctgatggcttattaccggcat

SEQ ID NO: 501 - DNA, 20 bp, Artificial Sequence
  Primer; splD; BPC144
agtgacatctgatgcggttg

SEQ ID NO: 502 - DNA, 21 bp, Artificial Sequence
  Primer; BPC145
aacaccaattgcttctcgctt

SEQ ID NO: 503 - DNA, 20 bp, Artificial Sequence
  Primer; dps; BPC146
agcggtaggaggaaaccctg

SEQ ID NO: 504 - DNA, 22 bp, Artificial Sequence
  Primer; BPC147
gttctgcagagtaacctttcgc

SEQ ID NO: 505 - DNA, 21 bp, Artificial Sequence
  Primer; srtB; BPC846
tgagcgagaacatcgacgtaa

SEQ ID NO: 506 - DNA, 20 bp, Artificial Sequence
  Primer; BPC847
ccgacatggtgcccgtataa

SEQ ID NO: 507 - DNA, 21 bp, Artificial Sequence
  Primer; emp; BPC854
tcgcgtgaatgtagcaacaaa

SEQ ID NO: 508 - DNA, 21 bp, Artificial Sequence
  Primer; BPC855
acttctgggcctttagcaaca

SEQ ID NO: 509 - DNA, 20 bp, Artificial Sequence
  Primer; sbnA; BPC858
cctggaggcagcatgaaaga

SEQ ID NO: 510 - DNA, 20 bp, Artificial Sequence
  Primer; BPC859
cattgccaacgcaatgccta

SEQ ID NO: 511 - DNA, 20 bp, Artificial Sequence
  Primer; CH52_360; BPC834
ttcaactcgaacgctgacga

SEQ ID NO: 512 - DNA, 20 bp, Artificial Sequence
  Primer; BPC835
ttgcacccattgttgcacct

SEQ ID NO: 513 - DNA, 20 bp, Artificial Sequence
  Primer; CH52_305; BPC838
ttcctggagcagtaccacca

SEQ ID NO: 514 - DNA, 22 bp, Artificial Sequence
  Primer; BPC839
cagcgcaatcgctgttaaacta

SEQ ID NO: 515 - DNA, 20 bp, Artificial Sequence
  Primer; CH521670; BPC842
gcgattatgggaccaaacgg

SEQ ID NO: 516 - DNA, 21 bp, Artificial Sequence
  Primer; BPC843
acttcatagcttgggtgtccc

SEQ ID NO: 517 - DNA, 20 bp, Artificial Sequence
  Primer; clfA; BPC850
tccagcacaacaggaaacga

SEQ ID NO: 518 - DNA, 20 bp, Artificial Sequence
  Primer; BPC851
tagcttcaccagttaccggc

SEQ ID NO: 519 - DNA, 20 bp, Artificial Sequence
  Primer; SAUSA300_2268; BPC778
gcttctacagctttgccgat

SEQ ID NO: 520 - DNA, 21 bp, Artificial Sequence
  Primer; BPC779
gatttggtgcttactgccacc

SEQ ID NO: 521 - DNA, 20 bp, Artificial Sequence
  Primer; SAUSA300_2616; BPC774
acaagcgcaacaagcaagag

SEQ ID NO: 522 - DNA, 23 bp, Artificial Sequence
  Primer; BPC775
tgcgtttgatacctttaacacgg

SEQ ID NO: 523 - DNA, 21 bp, Artificial Sequence
  Primer; SAUSA300_2617; BPC152
gggctgaaaaagttggcatga

SEQ ID NO: 524 - DNA, 20 bp, Artificial Sequence
  Primer; BPC153
acgcgttgtttttgacctcc

SEQ ID NO: 525 - DNA, 21 bp, Artificial Sequence
  Primer; hlgA2; BPC179
tgatttctgcaccttgaccga

SEQ ID NO: 526 - DNA, 20 bp, Artificial Sequence
  Primer; BPC180
agcccctttagccaatccat

SEQ ID NO: 527 - DNA, 22 bp, Artificial Sequence
  Primer; hrtAB; BPC713
acacaacaacaacgtgatgagc

SEQ ID NO: 528 - DNA, 20 bp, Artificial Sequence
  Primer; BPC714-
taacggtgcttgctctgctt

SEQ ID NO: 529 - DNA, 40 bp, Artificial Sequence
  Primer; DR_357
gagttgttgatggctaagtagacgcaatacaaaataggtg

SEQ ID NO: 530 - DNA, 38 bp, Artificial Sequence
  Primer; DR_410
cctgggtaccagtcatcaagcacagtttgactggaaag

SEQ ID NO: 531 - DNA, 29 bp, Artificial Sequence
  Primer; DR_359
ggaaccgattgaagggattcatttcgttg

SEQ ID NO: 532 - DNA, 34 bp, Artificial Sequence
  Primer; DR_409
ctcggttgctgtgttgcacacagttatctgtgag

SEQ ID NO: 533 - DNA, 42 bp, Artificial Sequence
  Primer; DR_361
tgcgtctacttagccatcaacaactctcctggcgcaccatcg

SEQ ID NO: 534 - DNA, 33 bp, Artificial Sequence
  Primer; DR_362
gtttcagggtttgcagactgatattcaatgacg

SEQ ID NO: 535 - DNA, 25 bp, Artificial Sequence
  Primer; DR_371
acatagcgcacgtagaacaacgacg

SEQ ID NO: 536 - DNA, 29 bp, Artificial Sequence
  Primer; DR_372
gccatctgtaaatcttgcgccattagtcc

SEQ ID NO: 537 - DNA, 39 bp, Artificial Sequence
  Primer; DR_407
gtgtgcaacacagcaaccgagcgttctgaacaaatccag

SEQ ID NO: 538 - DNA, 40 bp, Artificial Sequence
  Primer; DR_408
gtgcttgatgactggtacccaggaaacagctatgaccatg

SEQ ID NO: 539 - DNA, 40 bp, Artificial Sequence; identical to SEQ ID NO: 459
  Primer; DR_117

SEQ ID NO: 540 - DNA, 32 bp, Artificial Sequence; identical to SEQ ID NO: 335
  Primer; DR_228

SEQ ID NO: 541 - DNA, 26 bp, Artificial Sequence
  Primer; DR_116
ccctgttgataccgggaagccctggg

SEQ ID NO: 542 - DNA, 1084 bp, Artificial Sequence; identical to SEQ ID NO: 380
  Escherichia coli kanR fragment; BP_DNA_015

SEQ ID NO: 543 - DNA, 446 bp, Artificial Sequence; identical to SEQ ID NO: 344
  PsprA1(as)-sprA1(as); BP_DNA_018

SEQ ID NO: 544 - DNA, 40 bp, Artificial Sequence; identical to SEQ ID NO: 459
  Primer; DR_117

SEQ ID NO: 545 - DNA, 40 bp, Artificial Sequence; identical to SEQ ID NO: 460
  Primer; DR_254

SEQ ID NO: 546 - DNA, 29 bp, Artificial Sequence
  Primer; DR_533
gattacgcttacattcgcttctctgtttc

SEQ ID NO: 547 - DNA, 30 bp, Artificial Sequence
  Primer; DR_534
cagctgttgataatgccatttttgcacgag

SEQ ID NO: 548 - DNA, 22 bp, Artificial Sequence
  Primer; BP_964
tcaaacttcagcaggttctagc

SEQ ID NO: 549 - DNA, 22 bp, Artificial Sequence
  Primer; BP_965
gtaccaggtatgactgaatgcc

SEQ ID NO: 550 - DNA, 48 bp, Artificial Sequence; identical to SEQ ID NO: 448
  Primer; BP_949

SEQ ID NO: 551 - DNA, 32 bp, Artificial Sequence; identical to SEQ ID NO: 335
  Primer; DR_228

SEQ ID NO: 552 - DNA, 42 bp, Artificial Sequence; identical to SEQ ID NO: 449
  Primer; BP_950

SEQ ID NO: 553 - DNA, 32 bp, Artificial Sequence
  Primer; DR_318
cgattacttcccaaccattacctactgtcaac

SEQ ID NO: 554 - DNA, 55 bp, Artificial Sequence
  Primer; BM_049
cgtactgattgggtaggtgacatatagccgcaccaataaaaattgataatagctg

SEQ ID NO: 555 - DNA, 40 bp, Artificial Sequence
  Primer; BM_015
ggctatatgtcacctacccaatcagtacgttaattttggc

SEQ ID NO: 556 - DNA, 40 bp, Artificial Sequence
  Primer; BM_014
ggtgtataaggtgatggtaagccgatacgtacccgatatg

SEQ ID NO: 557 - DNA, 37 bp, Artificial Sequence
  Primer; BM_013
tcggcttaccatcaccttatacacctcctctctgcgg

SEQ ID NO: 558 - DNA, 49 bp, Artificial Sequence
  Primer; DR_634
caggagagttgttgatgcatgtaactgggcagtgtcttaaaaaatcgac

SEQ ID NO: 559 - DNA, 37 bp, Artificial Sequence
  Primer; DR_636
cagttacatgcatcaacaactctcctggcgcaccatc

SEQ ID NO: 560 - DNA, 44 bp, Artificial Sequence
  Primer; BM_052
ggtgtataaggtgatgattcaaagggatattgaatactcgggac

SEQ ID NO: 561 - DNA, 31 bp, Artificial Sequence
  Primer; BM_027
gctatatgtcacttacccaaagagcgccgcg

SEQ ID NO: 562 - DNA, 35 bp, Artificial Sequence
  Primer; BM_025
ccctttgaatcatcaccttatacacctcctctctg

SEQ ID NO: 563 - DNA, 42 bp, Artificial Sequence
  Primer; BM_024
gctctttgggtaagtgacatatagccgcaccaataaaaattg

SEQ ID NO: 564 - DNA, 41 bp, Artificial Sequence
  Primer; BM_018
ggtgtataaggtgatggcgtattttctggattttgacgagc

SEQ ID NO: 565 - DNA, 38 bp, Artificial Sequence
  Primer; BM_019
ggctatatgtcactcagagaatgcgtttgaccgcctcg

SEQ ID NO: 566 - DNA, 37 bp, Artificial Sequence
  Primer; BM_017
aaaatacgccatcaccttatacacctcctctctgcgg

SEQ ID NO: 567 - DNA, 41 bp, Artificial Sequence
  Primer; BM_016
cgcattctctgagtgacatatagccgcaccaataaaaattg

SEQ ID NO: 568 - DNA, 28 bp, Artificial Sequence
  Primer; DR_244
catcaccttatacacctcctctctgcgg

SEQ ID NO: 569 - DNA, 50 bp, Artificial Sequence
  Primer; DR_661
ctgaggagtaagtgacatatagccgcaccaataaaaattgataatagctg

SEQ ID NO: 570 - DNA, 52 bp, Artificial Sequence
  Primer; DR_659
cgcagagaggaggtgtataaggtgatgaagcagcaaaaggcgatgttaatcg

SEQ ID NO: 571 - DNA, 47 bp, Artificial Sequence
  Primer; DR_660
gtgcggctatatgtcacttactcctcaggttcgtaagctgtgaagac

SEQ ID NO: 572 - DNA, 36 bp, Artificial Sequence
  Primer; DR_674
gtccaggtaagtacccaggaaacagctatgaccatg

SEQ ID NO: 573 - DNA, 39 bp, Artificial Sequence
  Primer; DR_673
agctgtttcctgggtacttacctggacgtgcaggccatg

SEQ ID NO: 574 - DNA, 40 bp, Artificial Sequence
  Primer; DR_672
ggaggtgtataaggtgatgaagcacaaccctctggtggtg

SEQ ID NO: 575 - DNA, 38 bp, Artificial Sequence
  Primer; DR_675
ggttgtgcttcatcaccttatacacctcctctctgcgg

SEQ ID NO: 576 - DNA, 38 bp, Artificial Sequence; identical to SEQ ID NO: 332
  Primer; DR_280

SEQ ID NO: 577 - DNA, 45 bp, Artificial Sequence
  Primer; DR_278
cgcagagaggaggtgtataaggtgatgcttattttcgttcacatc

SEQ ID NO: 578 - DNA, 32 bp, Artificial Sequence; identical to SEQ ID NO: 335
  Primer; DR_228

SEQ ID NO: 579 - DNA, 26 bp, Artificial Sequence
  Synthetic construct; BP_542
catcaccttatacacctcctctctgc

SEQ ID NO: 580 - DNA, 27 bp, Artificial Sequence
  Synthetic construct; BP_717
actctttgaagtcattctttacaggag

SEQ ID NO: 581 - DNA, 27 bp, Artificial Sequence
  Synthetic construct; BP_718
ctcctgtaaagaatgacttcaaagagt

SEQ ID NO: 582 - DNA, 30 bp, Artificial Sequence
  Synthetic construct; DR_215
ccgacctcattaagcagctctaatgcgctg

SEQ ID NO: 583 - DNA, 29 bp, Artificial Sequence
  Synthetic construct; DR_216
ggtgtgaaataccgcacagatgcgtaagg

SEQ ID NO: 584 - DNA, 51 bp, Artificial Sequence
  Synthetic construct; DR_725
gcaataaaaaataagtgacatatagccgcaccaataaaaattgataatagc

SEQ ID NO: 585 - DNA, 60 bp, Artificial Sequence
  Synthetic construct; DR_726
ggtgcggctatatgtcacttattttttattgcttaaatttattattgctactactatacc

SEQ ID NO: 586 - DNA, 49 bp, Artificial Sequence
  Synthetic construct; DR_727
cgcagagaggaggtgtataaggtgatgatatctattgcaaacgcattac

SEQ ID NO: 587 - DNA, 58 bp, Artificial Sequence
  Synthetic construct; DR_728
tggtgcggctatatgtcacttattttttatggtcttgagtactaatcaatactaaacc

SEQ ID NO: 588 - DNA, 52 bp, Artificial Sequence
  Synthetic construct; DR_729
cgcagagaggaggtgtataaggtgatgtctgattttgaaatgctgatggttg

SEQ ID NO: 589 - DNA, 54 bp, Artificial Sequence
  Synthetic construct; DR_730
gaccataaaaaataagtgacatatagccgcaccaataaaaattgataatagctg

SEQ ID NO: 590 - DNA, 31 bp, Artificial Sequence
  Synthetic construct; DR_733
gtgcggctatatgtcacttattttttattgc

SEQ ID NO: 591 - DNA, 21 bp, Artificial Sequence
  Synthetic construct; DR_734
cgcagagaggaggtgtataag

SEQ ID NO: 592 - DNA, 81 bp, Artificial Sequence; identical to SEQ ID NO: 401
  Synthetic construct; sprG3 BP_164

SEQ ID NO: 593 - DNA, 108 bp, Artificial Sequence; identical to SEQ ID NO: 434
  Synthetic construct; sprG2 BP_165

SEQ ID NO: 594 - DNA, 164 bp, Artificial Sequence
  BP_DNA_150; sprA1 from p174
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaattgataatagctgagcccggg

SEQ ID NO: 595 - DNA, 6440 bp, Artificial Sequence
  BP_DNA_151; p151 linearized; p174 Plasmid backbone
cactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcggacagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggtttttaggcataaaactatatgatttacccctaaatctttaaaatgccccttaaaattcaaaataaaggcatttaaaatttaaatatttcttgtgataaagtttgttaaaaaggagtggttttatgactgttatgtggttatcgattataggtatgtggttttgtattggaatggcattttttgctatcaaggttattaaaaataaaaattagaccacgcatttatgccgagaaaatttattgtgcgttgagaagaacccttaactaaacttgcagacgaatgtcggcatagcgtgagctattaagccgaccattcgacaagttttgggattgttaagggttccgaggctcaacgtcaataaagcaattggaataaagaagcgaaaaaggagaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccggcgaggctagttacccttaagttattggtatgactggttttaagcgcaaaaaaagttgctttttcgtacctattaatgtatcgttttaaatgaatagtaaaaaacatacatagaaaggggaaaaagcaactttttttattgtcatagtttgtgaaaactaagttgtttttatgtgttataacatggaaaagtatactgagaaaaaacaaagaaatcaagtatttcagaaatttattaaacgtcatattggagagaatcaaatggatttagttgaagattgcaatacatttctgtcttttgtagctgataaaactttagaaaaacagaaattatataaagctaattcttgtaaaaatcgattttgtcctgtctgtgcttggagaaaagctagaaaagatgcattgggtttatctttgatgatgcaatatattaagcagcaagagaaaaaggagtttatctttttaactttgactacacctaatgtaatgagtgatgaattagaaaatgaaataaaacgttataataattcttttagaaaacttataaagagaaaaaaagtaggtagtgttataaagggatatgttcgtaagttagagattacatataataaaaaaagagatgattataatcctcattttcatgtgttaattgcagtaaataaatcgtatttcacagataaaagatattatattagccaacaagaatggttagatttatggcgtgatgtaacgggcatttcagaaataacacaagttcaagttcaaaaaataagacaaaataataataaagaattatatgaaatggctaagtattctggtaaagatagtgattatttaataaatcaaaaagtctttgatgcattttataaatcacttaaaggtaaacaggtattagtttattcaggattatttaaagaggctaaaaagaaattaaaaaatggggatttagattacttaaaagaaattgatccaaccgaatatatctatcaaattttttatatttggaaacaaaaagagtatttagctagtgaactttatgacttaacagaacaagaaaaaagagaaattaatcacaaaatgatagacgaaatcgaggaagaacaataacaaaatataagtgctaacagctgacctcccgataacaccatgtagttattgggaggtcagctgttgaattatgcacgagtattttaaaagttattgtgatgacgacgataaacgattatcaaaagtataatgttaaaatgctttattatactaacgttatataaacattatactttcgttatacaaattttaaccctgttaggaactataaaaaatcatgaaaattttaatttgcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagc

SEQ ID NO: 596 - protein, 30 aa, Artificial Sequence; identical to SEQ ID NO: 104
  BP_AA_002; sprA1

SEQ ID NO: 597 - DNA, 51 bp, Artificial Sequence
  Primer DR_476
ccgcagagaggaggtgtataaggtgatgagtaaaggagaagaacttttcac

SEQ ID NO: 598 - DNA, 58 bp, Artificial Sequence
  Primer DR_247
caatttttattggtgcggctatatgtcacttatttgtatagttcatccatgccatgtg

SEQ ID NO: 599 - DNA, 43 bp, Artificial Sequence
  Primer DR_245
gtgacatatagccgcaccaataaaaattgataatagctgagcc

SEQ ID NO: 600 - DNA, 144 bp, Artificial Sequence; identical to SEQ ID NO: 274
  sprA1 BP_DNA_150 toxin

(19) United States
(12) Patent Application Publication
(10) Pub. No.: US 2022/0288135 A1
(43) Pub. Date: Sep. 15, 2022

(54) LIVE BIOTHERAPEUTIC COMPOSITIONS AND METHODS

(71) Applicant: BioPlx, Inc., Boulder, CO (US)

(72) Inventors: Timothy W. STARZL, Boulder, CO (US); Ravi S. V. STARZL, Boulder, CO (US)

(73) Assignee: BioPlx, Inc., Boulder, CO (US)

(21) Appl. No.: 17/625,040

(22) PCT Filed: Jul. 8, 2020
(86) PCT No.: PCT/US2020/041237

§ 371 Date: Jan. 5, 2022

Related U.S. Application Data
(60) Provisional application No. 62/871,527, filed on Jul. 8, 2019.

(51) Int. Cl. A61K 35/74; A61K 36/06; A01N 63/22; A61P 31/00

(52) U.S. Cl. CPC A61K 35/74; A61K 36/06; A01N 63/22; A61P 31/00

(57) ABSTRACT

Live biotherapeutic compositions and methods are provided for treatment, prevention, or prevention of recurrence of skin and soft tissue infections, such as mastitis and/or intramammary infections, for example, in cows, goats, sows, and sheep. Methods include decolonizing and durably replacing with a live biotherapeutic composition comprising a synthetic microorganism that may safely and durably replace an undesirable microorganism under dermal, mucosal, or intramammary conditions.

### CROSS REFERENCES TO RELATED APPLICATIONS

This application is being filed on 8 Jul. 2020 as a PCT International Patent application and claims the benefit of priority to U.S. Provisional Application Ser. No. 62/871,527, filed 8 Jul. 2019, which is incorporated herein by reference in its entirety.

### SEQUENCE LISTING

The present application includes a Sequence Listing in electronic format as a txt file entitled “Sequence Listing 17814-0008WOU1.” which was created on 8 Jul. 2020 and which has a size of 312 kilobytes (KB) (319,496 bytes). The contents of txt file “Sequence Listing 17814-0008WOU1” are incorporated by reference herein.

### BACKGROUND

### Field

Methods and live biotherapeutic compositions are provided for treatment, prevention, or prevention of recurrence of dermal and or mucosal infections in a subject. In some embodiments, compositions and methods are provided for treating, preventing and or preventing recurrence of mastitis and/or intramammary infections in cows, goats, sows, and sheep. Methods are provided for durably influencing microbiological ecosystems (microbiomes) in the subject in order to resist infection and reduce recurrence of infection by an undesirable microorganism by decolonizing and durably replacing with a live biotherapeutic composition. Live biotherapeutic compositions are provided comprising a synthetic microorganism that may safely and durably replace an undesirable microorganism under intramammary, dermal or mucosal conditions. Synthetic microorganisms are provided containing molecular modifications designed to enhance safety, for example, by self-destructing when exposed to systemic conditions, by reducing the potential for acquisition of virulence or antibiotic resistance genes, and/or by producing a desirable product at the site of the ecosystem in a subject. Live biotherapeutic compositions are provided comprising synthetic microorganisms (e.g., live biotherapeutic products) that exhibit functional stability over at least 500 generations, and are useful in the treatment, prevention, or prevention of recurrence of microbial infections.

### Description of the Related Art

Mastitis is a persistent problem in dairy herds. Substantial economic costs and negative impact on animal health and welfare may occur. Mastitis is an inflammation of the mammary gland that originates from intramammary infection (IMI), most often caused by bacteria such as staphylococci, streptococci, and coliforms. Bacterial strains commonly associated with mastitis and intramammary infection include Staphylococcus aureus, coagulase-negative staphylococcus, Escherichia coli, Streptococcus uberis, and Streptococcus dysgalactiae. These bacterial strains have been treated using a broad-spectrum antibiotic. Problems with this approach include milk contamination, recurrence of infection, and development of antibiotic resistance.

One known approach, for example, to eliminate pre-partum intramammary infections (IMI) in heifers involves intramammary broad-spectrum antibiotic therapy shortly before or at the time of calving. However, problems with use of a broad-spectrum antibiotic include emergence of antibiotic resistant microorganisms and milk contamination with antibiotics. Inappropriate use of antibiotics may also lead to mismanagement of the microbiome in the animal.

Another known approach to prevent mastitis is use of commercially available vaccines for immunization against mastitis caused by Staphylococcus aureus and E. coli. For example, a Staph aureus bacterin marketed to U.S. dairy producers is LYSIGIN® (formerly Somato-Staph®), Boehringer ingelheim Vetmedica, Inc., which is labeled as somatic antigen containing phage types I, II, III, IV and miscellaneous groups of Staph aureus. LYSIGIN® is indicated for the vaccination of healthy, susceptible cattle as an aid in the prevention of mastitis caused by Staphylococcus aureus. There have been several commercially coliform mastitis vaccines including, for example, ENVIRACOR™ J-5 Bacterin, Zoetis; and J-VAC®, Merial/Boehringer-Ingleheim, an Escherichia coli bacterin-toxoid vaccine commercially available for protecting cows from coliform mastitis which can be used for lactating cows, heifers, or dry cows. Another gram negative mastitis vaccine (ENDOVAC-Bovi®, Endovac Animal Health) contains re-17 mutant Salmonella typhimurium bacterin toxoid with ImmunePlus® adjuvant. These coliform mastitis vaccine formulations each use gram-negative core antigens to produce non-specific immunity directed against endotoxic disease.

One of the most frustrating mastitis pathogens is Staphylococcus aureus. This organism is a highly successful mastitis pathogen in that it has evolved to produce infections of long duration with limited clinical signs. Infections with this pathogen may be subclinical in nature resulting, and may result in reduced yield and/or poor quality milk. Unfortunately, commercially available Staphylococcus aureus vaccines appear to have limited ability to prevent new infections. Ruegg 2005, Milk Money, Evaluating the effectiveness of mastitis vaccines; Middleton et al., Vet Microbiol 2009 Feb. 16; 134(1-2):192-8.

Alternative compositions and methods for prevention and treatment of mastitis and/or intramammary infection in cows, goats, sows and sheep are desirable.

Each individual is host to a vast population of trillions of microorganisms, composed of perhaps 10,000 different species, types and strains. These “commensal” organisms are found both on external sites (e.g. dermal) and on internal sites (e.g. gastrointestinal). “Colonization” happens automatically through ongoing interactions with the environment.

The menagerie of microorganisms constitutes the “biome”, a dynamic, structured, living system that in many cases, and in many ways, is essential for health and wellness. A biomic structure is created by a vast combinatorial web of relationships between the host, the environment, and the components of the biome. The animal microbiome is an ecosystem. It has a dynamic but persistent structure—it is “resilient” and has a “healthy” normal base state.

Nonetheless, under some circumstances the microbiome can be invaded and occupied by pathogenic microorganisms. This type of “colonization” may become a precursor to “infection”. This kind of disruption to the microbiome can cause serious and even life-threatening disease.

One unintended consequence of the mismanagement of the biome has been the emergence of “antibiotic resistance”. This happens when antibiotics and antiseptics do not fully eliminate the target microorganisms. The few survivors that show resistance to these materials then preferentially grow back (“recolonize”) into an open environment (or vacated “niche”) already cleared of competing organisms. The survivor organisms then dominate the space, usually retaining that resistance for their descendants. If exposed to a new killing agent they will tend to develop resistance to that as well. Over only a few generations these microorganisms can develop resistance to many or all of our known antibiotics, becoming the now famous “super-bugs”, and along the way creating an enormous new global health problem.

A phenomenon called “recurrence” is at the heart of the process that creates antibiotic resistance. While methods to treat pathogenic infection exist, methods to prevent recurrence are effectively nonexistent.

Bacterial infections are the home territory of the emerging “super bug” phenomenon. The overuse and misuse of antibiotics has caused many strains of pathogenic bacteria to evolve resistance to an increasing number of antibiotic therapies, creating a massive global public health problem. As each new variation of antibiotic is applied to treat these superbugs, the inevitable process of selecting for resistant strains begins anew, and resistant variants of the pathogen quickly develop. Unfortunately, today bacteria are becoming resistant faster than new antibiotics can be developed.

Beyond cultivating antibiotic resistance, and frequently causing adverse health effects in the recipients, antibiotic treatments also have the undesirable effect of disrupting the entire microbiome, including both good and bad bacteria. This often creates new problems such as opening the microbiome to colonization by adventitious pathogens after the treatment.

Bacteria however have less leeway to adapt to different resources, as these requirements are more basic on a molecular level and are intrinsically defined in the genome. This allows the microbiome ecology to behave as more of an “ideal” system, leading to full exclusion of one of the identical strain competitors from the niche.

The community of organisms colonizing the animal body is referred to as the microbiome. The microbiome is often subdivided for analysis into sections of geography (i.e. the skin microbiome versus the gastrointestinal microbiome) or of phylogeny (i.e. bacterial microbiome versus the fungal or protist microbiome).

Antibiotics are life-saving medicines, but they can also change, unbalance, and disrupt the microbiome. The microbiome is a community of naturally-occurring germs in and on the body—on skin, gut, mouth or respiratory tract, and in the urinary tracts. A healthy microbiome helps protect from infection. Antibiotics disrupt the microbiome, eliminating both “good” and “bad” bacteria. Drug-resistant bacteria-like MRSA, CRE, and C. difficile—can take advantage of this disruption and multiply. With this overgrowth of resistant bacteria, the body is primed for infection. Once subjects are colonized with resistant bacteria, the resistant bacteria can easily be spread to others. See “Antibiotic Resistance (AR) Solutions Initiative: Microbiome, CDC Microbiome Fact Sheet 2016”. www.cdc.gov/drugresistance/solutions-initiative/innovations-to-slow-AR.html.

Staphylococcus aureus colonizes about 30 to 50% of the human population. Sometimes friendly (commensal) and sometimes not (pathogenic), Staph aureus is ubiquitous, persistent, and is becoming increasingly virulent and drug resistant. Methicillin Resistant Staphylococcus aureus (MRSA) and virulent Methicillin Susceptible Staphylococcus aureus (v-MSSA) are increasingly found in bovine mastitis outbreaks. MRSA is now a threat to dairy workers, farmers, and veterinarians. Unfortunately, decolonization with antibiotics is of limited efficacy in preventing recurrence, and about 70% recurrence of MRSA and v-MSSA has been noted in several human studies. Kaur et al., 2017, American Academy of Pediatrics News, Developing guidelines for S. aureus decolonization a difficult task. https://www.aappublications.org/news/2017/05/01/Decolonization050117. Creech et al., Infect Dis Clin North Am 2015 September; 29(3): 429-464.

The FDA's Center for Veterinary Medicine (CVM) has revealed its 5-year plan to address antimicrobial stewardship in veterinary settings. According to the agency, the plan builds on the steps the CVM has taken to eliminate production uses of medically important antimicrobials—such as those used to treat human disease—and to bring all other therapeutic uses of antimicrobials under the oversight of licensed veterinarians. https://www.americanveterinarian.com/news/fda-unveils-5 year-plan-to-fight-antimicrobial-resistance, September 2018.

As antibiotics become more restricted, the absolute need for their effect is growing rapidly. Bovine strains may cross to human hosts, and human strains may cross to bovine hosts, and there is an increasing incidence and prevalence of antibiotic resistance. And with the appearance of these new and more virulent strains, new kinds of problems for herd health management will also appear.

It is not all just about animal productivity, public health concerns may also drive regulatory environment. Pasteurization of milk kills the bugs, but not the freed (by lysis) genetic elements. Horizontal gene transfer of mobile genetic elements may be possible. In vivo transformation may occur and has been demonstrated in the laboratory (data not shown). Methods for preventing mastitis and intramammary infection are desirable.

Virtually every microorganism may be a potential “accidental pathogen”, because even a “passive” microorganism can kill if it gets under the skin. This can occur via a cut, scratch, abrasion, surgery, injections, in-dwelling lines, etc. Bacteremia, septicemia, endocarditis, deep tissue and joint infections, intramammary infections, and skin and soft tissue infections (SSTIs) may occur.

Prior art methods employing suppression (decolonization) alone—such as use of antibiotics and antimicrobial agents—often fail because they are subject to high rates of recurrence. Decolonization is often insufficient when used alone to effectively prevent recurrence and/or transmission of the drug-resistant microorganism.

Among pathogenic microorganisms causing health care related infection in humans, methicillin-resistant Staphylococcus aureus (MRSA) has been given priority because of its virulence and disease spectrum, multidrug resistant profile and increasing prevalence in health care settings. MRSA is the most common cause of ventilator-associated pneumonia and surgical site infection and the second most common cause of central catheter associate bloodstream infection.

Decolonization alone has been used in hospital patients in an attempt to reduce transmission and prevent disease in Staphylococcus aureus carriers. Decolonization may involve a multi-day regimen of antibiotic and/or antiseptic agents—for example, intranasal mupirocin and chlorhexidine bathing. Universal decolonization is a method employed by some hospitals where all intensive care unit (ICU) hospital patients are washed daily with chlorhexidine and intranasal mupirocin, but since its widespread use, MRSA infection rates in the U.S. have not significantly changed. In addition, microorganisms may develop resistance to chlorhexidine and mupirocin upon repeated exposure.

Decolonization when used alone may not be durable because the vacated niche may become recolonized with pathogenic or drug-resistant microorganisms. This has been demonstrated in several human studies.

For example, Shinefield et al., 1963, Amer J Dis Child 105, June 1963, 146-154, observed that colonization of newborn infants with strains of Staphylococcus aureus of the 52/52a/80/81 phage complex by contact with a carrier was often followed by disease in babies and their family contacts. Shinefield also observed that control measures using antiseptic or antimicrobial agents applied to the infant lead to colonization with abnormal flora, consisting primarily of highly resistant coagulase negative staphylococci and Gram-negative organisms such as Pseudomonas and Proteus. Shinefield attempted to solve the problem by artificially colonizing newborns with staphylococcal strain 502a by nasal and/or umbilical inoculation. 502a is a coagulase positive strain of Staphylococcus aureus of low virulence, susceptible to penicillin, and incapable of being induced to produce beta-lactamase. It was shown that presence of other staphylococci interfered with acquisition of 502a. Persistence of colonization was at best 35% after 6 months to one year.

Boris M. et al, “Bacterial Interference: Protection Against Recurrent Intrafamilial Staphylococcal Disease.” Amer J Dis Child 115 (1968): 521-29, deliberately colonized ˜4000 infants in first few hours of life with Staphylococcus aureus 502a (nares & umbilical stump). Virtually complete protection of babies from 80/81 infection was observed (babies were monitored for 1-year post inoculation). Although 5-15% of babies developed tiny treatment emergent vesicles that self-resolved in first 3 days post-treatment. Prior decolonization improves persistence of 502a up to 5-fold compared to placebo (saline) n=63. Controlled studies in recurrent furunculosis showed that decolonization with systemic antibiotics+nasal antimicrobial followed by application of 502a curtailed disease in 80% of patients.

Recolonization with a drug-susceptible strain may not be safe because the drug-susceptible strain may still cause systemic infection.

In one human study, Shinefield et al., 1973, Microbiol Immunol, vol. 1, 541-547, reported using bacterial replacement including decolonization in treating patients with recurrent furunculosis. Chronic staphylococcal carriers were treated with antibiotic therapy including systemic antibiotics and application of antimicrobial cream to nasal mucosa. In an initial study, 31 patients received antibiotic therapy alone and exhibited a 74% recurrence rate of original strain. 18 patients received antibiotic treatment followed by 502a inoculation and exhibited 27% recurrence of original strain. A larger study of 587 patients resulted in 21% recurrence of original strain after 12 months. However, a high relapse rate was noted in patients with diabetes, eczema or acne. Disease associated with 502a was noted in 11 patients.

In another human study, Aly et al., 1974 J Infect Dis 129(6) pp. 720-724, studied bacterial interference in carriers of Staphylococcus aureus. The carriers were treated with antibiotics and antibacterial soaps and challenged with strain 502a. Specifically, decolonization method involved oral dicloxacillin 8 days; neosporin in nose for 8 days, and trichlorocarbanilide. It was found that full decolonization was needed to get good take. Day 7 showed 100% take, but at day 23 the take was down to 60 to 80%. The persistence data was 73% at 23 weeks for well-decolonized subjects, and only 17% persistence for partially decolonized subjects. Co-colonization was found in 5/12 subjects at day 3, 2/12 subjects at day 10, and 1/12 subjects at day 35 and at day 70. Decolonization, followed by recolonization with a microorganism of the same genus, but a different species, may not be durable because the vacated niche is not adequately filled by the different species.

WO2009117310 A2, George Liu, assigned to Cedars-Sinai Medical Center, discloses methods for treatment and prevention of methicillin-resistant Staphylococcus aureus and methicillin-sensitive Staphylococcus aureus (MSSA) using a decolonization/recolonization method. In one example, mice are treated with antibiotics to eradicate existing flora, including MRSA, and newly cleared surface area is colonized with bacteria of the same genus, but of a different species, such as Staphylococcus epidermidis. No specific data regarding recurrence is provided.

Administration of probiotics in an attempt to treat infection by pathogenic microorganisms may not be effective and may not be durable because the probiotic may not permanently colonize the subject.

U.S. Pat. No. 6,660,262, Randy McKinney, assigned to Bovine Health Products, Inc., discloses broad spectrum antimicrobial compositions comprising certain minerals, vitamins, cobalt amino acids, kelp and a Lactobacillus species for use in treating microbial infection in animals. Field trials in cattle and horses were performed, but the infectious bacterial strain or other infectious agent was not identified.

U.S. Pat. No. 6,905,692, Sean Farmer, assigned to Ganeden Biotech, Inc., discloses topical compositions containing certain combinations of probiotic Bacillus bacteria, spores and extracellular products for application to skin or mucosa of a mammal for inhibiting growth of certain bacterium, yeast, fungi, and virus. Compositions comprising Bacillus coagulans spores, or Bacillus species. culture supernatants and Pseudomonas lindbergii culture supernatants in a vehicle such as emu oil are provided. The disclosure states since probiotics do not permanently colonize the host, they need to be ingested or applied regularly for any health-promoting properties to persist.

U.S. Pat. No. 6,461,607, Sean Farmer, assigned to Ganeden Biotech, Inc., discloses lactic acid-producing bacteria, preferably strains of Bacillus coagulans, for the control of gastrointestinal tract pathogens in a mammal. Methods for selective breeding and isolation of probiotic, lactic acid-producing bacterial strains which possess resistance to an antibiotic are disclosed. Methods for treating infections with a composition comprising an antibiotic-resistant lactic-acid producing bacteria and an antibiotic are disclosed.

U.S. Pat. No. 8,906,668, assigned to Seres Therapeutics, provide cytotoxic binary combinations of 2 or more bacteria of different operational taxonomic units (OTUs) to durably exclude a pathogenic bacterium. The OTUs are determined by comparing sequences between organisms, for example as sharing at least 95% sequence identity of 16S ribosomal RNA genes in at least in a hypervariable region.

Prior art methods employing replacement of the original pathogenic microorganism (recolonization) alone are subject to poor colonization rates with the new microorganism. The process may fail if the recolonization is done incorrectly. Effective recolonization is critical but not sufficient when used alone to prevent recurrence.

Prior art methods involving both suppression (decolonization) of the original pathogenic microorganism and replacement (recolonization) with a new microorganism may give variable recurrence of the pathogenic microorganism depending on the specific method.

Rather than waging an un-winnable war against commensal pathogenic or drug-resistant microorganisms, a better approach may be to manage the microbiome: to actively promote “good bugs” and their supporting system dynamics, while selectively suppressing the recurrence of specific pathogenic organisms. Improved methods to safely and durably prevent and reduce recurrence of infection by undesirable microorganisms, such as virulent, pathogenic and/or drug-resistant microorganisms, are desirable.

### SUMMARY OF THE INVENTION

Live biotherapeutic compositions are provided for treatment, prevention, and prevention of recurrence of intramammary infection and/or mastitis in cows, goats, sows and sheep. The compositions contain a unique synthetic microorganism with a genomically integrated self-destruct program. The self-destruct program may be activated in the presence of blood or serum, and is designed not to be able to cause a systemic infection. The self-destruct program may be activated in the presence of plasma or interstitial fluid, and is designed not to cause a skin and soft tissue infection (SSTI). In this manner, the microorganisms should not typically be able to be accidental pathogens. The biotheraputic microorganisms provided herein are designed to be safe microbiomic replacements for both frank and opportunistic pathogens.

Kill-switched microorganisms provided herein kill themselves in blood, serum and plasma. They can colonize, but they cannot infect.

A live biotherapeutic composition is provided for treatment or prevention of bovine, caprine, ovine, or porcine mastitis and/or intramammary infection comprising at least one synthetic microorganism, and a pharmaceutically acceptable carrier, wherein the synthetic microorganism comprises a recombinant nucleotide having at least one kill switch molecular modification comprising a first cell death gene which is operatively associated with a first regulatory region comprising an inducible first promoter, wherein the first inducible promoter exhibits conditionally high level gene expression of the recombinant nucleotide in response to exposure to blood, serum, plasma, or interstitial fluid of at least three fold increase of basal productivity.

The synthetic microorganism further may further include at least a second molecular modification (expression clamp) comprising an antitoxin gene specific for the first cell death gene, wherein the antitoxin gene is operably associated with a second regulatory region comprising a second promoter which is active (constitutive) upon dermal or mucosal colonization or in a complete media, but is not induced, induced less than 1.5-fold, or is repressed after exposure to blood, serum, plasma, or interstitial fluid for at least 30 minutes.

The at least one molecular modification may be integrated to a chromosome of the synthetic microorganism.

The first promoter may be upregulated by at least 5-fold, at least 10-fold, at least 20-fold, at least 50-fold, or at least 100-fold within at least 30 min, 60 min, 90 min, 120 min, 180 min, 240 min, 300 min, or at least 360 min following exposure to blood, serum, plasma, or interstitial fluid.

In some embodiments, the first promoter is not induced, induced less than 1.5 fold, or is repressed in the absence of blood, serum, plasma, or interstitial fluid.

The second regulatory region comprising a second promoter may be active upon dermal or mucosal colonization or in TSB media, but is repressed at least 2 fold upon exposure to blood, serum, plasma, or interstitial fluid after a period of time selected from the group consisting of the group consisting of at least 30 min, 60 min, 90 min, 120 min, 180 min, 240 min, 300 min, and at least 360 min.

Measurable average cell death of the synthetic microorganism occurs within at least a preset period of time following induction of the first promoter. The measurable average cell death may occur within at least a preset period of time selected from the group consisting of within at least 1, 5, 15, 30, 60, 90, 120, 180, 240, 300, or 360 min minutes following exposure to blood, serum, plasma, or interstitial fluid. The measurable average cell death may be at least a 50% cfu, at least 70%, at least 80%, at least 90%, at least 95%, at least 99%, at least 99.5%, at least 99.8%, or at least 99.9% cfu count reduction following the preset period of time.

The kill switch molecular modification may reduce or prevent infectious growth of the synthetic microorganism under systemic or SSTI conditions in the subject.

The synthetic microorganism may be derived from a target microorganism having the same genus and species as an undesirable microorganism causing bovine, caprine, ovine, or porcine mastitis or intramammary infection.

The target microorganism may be susceptible to at least one antimicrobial agent. The target microorganism may be selected from a bacterial and/or yeast target microorganism.

The target microorganism may be a bacterial species capable of colonizing a dermal and/or mucosal niche and is a member of a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

The target microorganism may be a yeast. The target microorganism may be a yeast species capable of colonizing a dermal and/or mucosal niche. The target microorganism may be may be a member of a genus selected from the group consisting of Candida and Cryptococcus.

The target microorganism may be a Staphylococcus aureus strain. The synthetic microorganism may be a Staphylococcus aureus strain and the molecular modification may include the cell death gene is selected from the group consisting of sprA1, sprA2, kpn1, sma1, sprG, relF, rsaE, yoeB, mazF, yeJM, or lysostaphin toxin gene.

The synthetic microorganism may be a Staphylococcus aureus strain and the molecular modification may include a cell death gene comprising a nucleotide sequence selected from the group consisting of SEQ ID NOs: 122, 124, 125, 126, 127, 128, 274, 275, 284, 286, 288, 290, 315, and 317, or a substantially identical nucleotide sequence.

The synthetic microorganism may be a Staphylococcus aureus strain and the inducible first promoter may comprises or be derived from a gene selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

The synthetic microorganism may be a Staphylococcus aureus strain and the first promoter may comprise a nucleotide sequence selected from the group consisting of SEQ ID NO: 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical nucleotide sequence thereof.

In some embodiments, the synthetic microorganism comprises an antitoxin gene encoding an antisense RNA sequence capable of hybridizing with at least a portion of the first cell death gene.

The antitoxin gene may be selected from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene. The antitoxin gene may comprise a nucleotide sequence selected from the group consisting of SEQ ID NOs: 273, 306, 307, 308, 309, 310, 311, 312, 314, 319, 322, 342, 347, 362, 364, 368, 373, 374, 375, 376, 377, and 378, or a substantially identical nucleotide sequence.

In some embodiments, the synthetic microorganism comprises a second promoter comprises or is derived from a gene selected from the group consisting of clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR family transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho. The second promoter may be derived from a P_{clfB} (clumping factor B) and may optionally comprise a nucleotide sequence of SEQ ID NO: 117, 118, 129 or 130, or a substantially identical nucleotide sequence thereof.

In some embodiments, a live biotherapeutic composition is provided comprising one or more, two or more, three of more, four or more, five or more, six or more, seven or more synthetic microorganisms selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa.

In some embodiments, the live biotherapeutic composition comprises a mixture of synthetic microorganisms comprising at least a Staphylococcus sp., a Escherichia sp., and a Streptococcus sp. synthetic strains.

A composition is provided for use in the manufacture of a medicament for eliminating and preventing the recurrence of bovine, caprine, or ovine mastitis, optionally comprising two or more, three or more, four or more, five or more, six or more, seven or more, eight or more, nine or more, or ten or more synthetic microorganisms.

In a particular embodiment, a biotherapeutic composition is provided comprising three or more synthetic microorganisms derived from target microorganisms including each of a Staphylococci species, a Streptococci species, and an Escherichia coli species.

The target Staphylococcus species may be selected from the group consisting of a catalase-positive Staphylococcus species and a coagulase-negative Staphylococcus species. The target Staphylococcus species may be selected from the group consisting of Staphylococcus aureus, S. epidermidis, S. chromogenes, S. simulans, S. saprophyticus, S. sciuri, S. haemolyticus, and S. hyicus. The target Streptococci species may be a Group A, Group B or Group C/G species. The target Streptococci species may be selected from the group consisting of Streptococcus uberis, Streptococcus agalactiae, Streptococcus dysgalactiae, and Streptococcus pyogenes. The E. coli species may be a Mammary Pathogenic Escherichia coli (MPEC) species.

A method is provided for treating, preventing, or preventing the recurrence of bovine, caprine, ovine, or porcine mastitis or intramammary infection associated with an undesirable microorganism in a subject hosting a microbiome, comprising: a. decolonizing the bovine, caprine, or ovine host microbiome; and b. durably replacing the undesirable microorganism by administering to the subject a biotherapeutic composition comprising a synthetic microorganism comprising at least one element imparting a non-native attribute, wherein the synthetic microorganism is capable of durably integrating to the host microbiome, and occupying the same niche in the host microbiome as the undesirable microorganism.

The decolonizing may be performed on at least one site in the bovine, caprine, or ovine subject to substantially reduce or eliminate the detectable presence of the undesirable microorganism from the at least one site.

The niche may be an intramammary, dermal, or mucosal environment that allows stable colonization of the undesirable microorganism at the at least one site.

Methods and compositions are provided for safely and durably influencing microbiological ecosystems (microbiomes) in a subject to perform a variety of functions, for example, including reducing the risk of infection by an undesirable microorganism such as virulent, pathogenic and/or drug-resistant microorganism.

Methods are provided herein to prevent or reduce the risk of colonization, infection, recurrence of colonization, or recurrence of a pathogenic infection by an undesirable microorganism in a bovine, caprine, ovine or porcine subject, comprising: decolonizing the undesirable microorganism on at least one site in the subject to reduce or eliminate the presence of the undesirable microorganism from the site; and durably replacing the undesirable microorganism by administering a synthetic microorganism to the at least one site in the subject, wherein the synthetic microorganism can durably integrate with a host microbiome by occupying the niche previously occupied by the undesirable microorganism; and optionally promoting colonization of the synthetic microorganism within the subject.

The disclosure provides a method for eliminating and preventing the recurrence of a undesirable microorganism in a bovine, caprine, ovine or porcine subject hosting a microbiome, comprising (a) decolonizing the host microbiome; and (b) durably replacing the undesirable microorganism by administering to the subject a synthetic microorganism comprising at least one element imparting a non-native attribute, wherein the synthetic microorganism is capable of durably integrating to the host microbiome, and occupying the same niche in the host microbiome as the undesirable microorganism.

In some embodiments, the decolonizing is performed on at least one site in the bovine, caprine, ovine or porcine subject to substantially reduce or eliminate the detectable presence of the undesirable microorganism from the at least one site.

In some embodiments, the detectable presence of an undesirable microorganism or a synthetic microorganism is determined by a method comprising a phenotypic method and/or a genotypic method, optionally wherein the phenotypic method is selected from the group consisting of biochemical reactions, serological reactions, susceptibility to anti-microbial agents, susceptibility to phages, susceptibility to bacteriocins, and/or profile of cell proteins. In some embodiments, the genotypic method is selected a hybridization technique, plasmids profile, analysis of plasmid polymorphism, restriction enzymes digest, reaction and separation by Pulsed-Field Gel Electrophoresis (PFGE), ribotyping, polymerase chain reaction (PCR) and its variants, Ligase Chain Reaction (LCR), and Transcription-based Amplification System (TAS).

In some embodiments, the niche is a dermal or mucosal environment that allows stable colonization of the undesirable microorganism at the at least one site in the subject.

In some embodiments, the ability to durably integrate to the host microbiome is determined by detectable presence of the synthetic microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the ability to durably replace the undesirable microorganism is determined by the absence of detectable presence of the undesirable microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the ability to occupy the same niche is determined by absence of co-colonization of the undesirable microorganism and the synthetic microorganism at the at least one site after the administering step. In some embodiments, the absence of co-colonization is determined at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the synthetic microorganism comprises at least one element imparting the non-native attribute that is durably incorporated to the synthetic microorganism. In some embodiments, the at least one element imparting the non-native attribute is durably incorporated to the host microbiome via the synthetic microorganism.

In some embodiments, the at least one element imparting the non-native attribute is a kill switch molecular modification, virulence block molecular modification, or nanofactory molecular modification. In some embodiments, the synthetic microorganism comprises molecular modification that is integrated to a chromosome of the synthetic microorganism. In some embodiments, the synthetic microorganism comprises a virulence block molecular modification that prevents horizontal gene transfer of genetic material from the undesirable microorganism.

In some embodiments, the measurable average cell death of the synthetic microorganism occurs within at least a preset period of time following induction of the first promoter after the change in state. In some embodiments, the measurable average cell death occurs within at least a preset period of time selected from the group consisting of within at least 1, 5, 15, 30, 60, 90, 120, 180, 240, 300, or 360 min minutes following the change of state. In some embodiments, the measurable average cell death is at least a 50% cfu, at least 70%, at least 80%, at least 90%, at least 95%, at least 99%, at least 99.5%, at least 99.8%, or at least 99.9% cfu count reduction following the preset period of time. In some embodiments, the change in state is selected from one or more of pH, temperature, osmotic pressure, osmolality, oxygen level, nutrient concentration, blood concentration, plasma concentration, serum concentration, metal concentration, chelated metal concentration, change in composition or concentration of one or more immune factors, mineral concentration, and electrolyte concentration. In some embodiments, the change in state is a higher concentration of and/or change in composition of blood, serum, or plasma compared to normal physiological (niche) conditions at the at least one site in the subject.

The undesirable microorganism may be selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa.

The biotherapeutic composition comprising a synthetic microorganism may be administered pre-partum, early, mid-, or late lactation phase or in the dry period to the cow, goat sheep, or sow in need thereof.

In some embodiments, the undesirable microorganism is a Staphylococcus aureus strain, and wherein the detectable presence is measured by a method comprising obtaining a sample from the at least one site of the subject, contacting a chromogenic agar with the sample, incubating the contacted agar and counting the positive cfus of the bacterial species after a predetermined period of time.

In some embodiments, a method is provided comprising a decolonizing step comprising topically administering a decolonizing agent to at least one site in the subject to reduce or eliminate the presence of the undesirable microorganism from the at least one site.

In some embodiments, the decolonizing step comprises topical administration of a decolonizing agent, wherein no systemic antimicrobial agent is simultaneously administered. In some embodiments, no systemic antimicrobial agent is administered prior to, concurrent with, and/or subsequent to within one week, two weeks, three weeks, one month, two months, three months, six months, or one year of the first topical administration of the decolonizing agent or administration of the synthetic microorganism. In some embodiments, the decolonizing agent is selected from the group consisting of a disinfectant, bacteriocide, antiseptic, astringent, and antimicrobial agent.

In some embodiments, the decolonizing agent is selected from the group consisting of alcohols (ethyl alcohol, isopropyl alcohol), aldehydes (glutaraldehyde, formaldehyde, formaldehyde-releasing agents (noxythiolin=oxymethylenethiourea, tauroline, hexamine, dantoin), o-phthalaldehyde), anilides (triclocarban=TCC=3,4,4′-trichlorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), chloroxylenol (PCMX), 8-hydroxyquinoline, dodecyl benzene sulfonic acid, nisin, chlorine, glycerol monolaurate, C₈-C₁₄ fatty acids, quaternary ammonium compounds (cetrimide, benzalkonium chloride, cetyl pyridinium chloride), silver compounds (silver sulfadiazine, silver nitrate), peroxy compounds (hydrogen peroxide, peracetic acid, benzoyl peroxide), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), isotretinoin, sulfur compounds, botanical extracts (peppermint, calendula, eucalyptus, Melaleuca spp. (tea tree oil), (Vaccinium spp. (e.g., A-type proanthocyanidins), Cassia fistula Linn, Baekea frutescens L., Melia azedarach L., Muntingia calabura, Vitis vinifera L, Terminalia avicennioides Guill & Perr., Phylantus discoideus muel. Muel-Arg., Ocimum gratissimum Linn., Acalypha wilkesiana Muell-Arg., Hypericum pruinatum Boiss.&Bal., Hypericum olimpicum L. and Hypericum sabrum L., Hamamelis virginiana (witch hazel), Clove oil, Eucalyptus spp., Rosmarinus officinalis spp. (rosemary), thymus spp. (thyme), Lippia spp. (oregano), lemongrass spp., cinnamomum spp., geranium spp., lavendula spp., calendula spp.), aminolevulinic acid, topical antibiotic compounds (bacteriocins; mupirocin, bacitracin, neomycin, polymyxin B, gentamicin).

In some embodiments, the antimicrobial agent is selected from the group consisting of cephapirin, amoxicillin, trimethoprim-sulfonamides, sulfonamides, oxytetracycline, fluoroquinolones, enrofloxacin, danofloxacin, marbofloxacin, cefquinome, ceftiofur, streptomycin, oxytetracycline, vancomycin, cefazolin, cephalothin, cephalexin, linezolid, daptomycin, clindamycin, lincomycin, mupirocin, bacitracin, neomycin, polymyxin B, gentamicin, prulifloxacin, ulifloxacin, fidaxomicin, minocycline, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norfloxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, and fluconazole.

In some embodiments, the decolonizing comprises topically administering the decolonizing agent at least one, two, three, four, five or six or more times prior to the replacing step. In some embodiments, the decolonizing step comprises administering the decolonizing agent to the at least one host site in the subject from one to six or more times or two to four times at intervals of between 0.5 to 48 hours apart, and wherein the replacing step is performed after the final decolonizing step.

The replacing step may be performed after the final decolonizing step, optionally wherein the decolonizing agent is in the form of a spray, dip, lotion, foam, cream, balm, or intramammary infusion.

In some embodiments, a method is provided comprising decolonizing an undesirable microorganism, and replacing with a synthetic microorganism comprising topical administration of a composition comprising at least 10⁵, at least 10⁶, at least 10⁷, at least 10⁸, at least 10⁹, at least 10¹⁰, or at least 10¹¹ CFU of the synthetic strain and a pharmaceutically acceptable carrier to at least one host site in the subject. In some embodiments, the initial replacing step is performed within 12 hours, 24 hours, 36 hours, 2 days, 3 days, 4 days, 5 days, 6 days, 7 days, 8 days, 9 days or 10 days, or between 0.5-10 days, 1-7 days, or 2 to 5 days of the decolonizing step. In some embodiments, the replacing step is repeated at intervals of no more than once every two weeks to six months following the final decolonizing step. In some embodiments, the decolonizing step and the replacing step is repeated at intervals of no more than once every two weeks to six months, or three weeks to three months. In some embodiments, the replacing comprises administering the synthetic microorganism to the at least one site at least one, two, three, four, five, six, seven, eight, nine, or ten times. In some embodiments, the replacing comprises administering the synthetic microorganism to the at least one site no more than one, no more than two, no more than three times, or no more than four times per month.

In some embodiments, the method of decolonizing the undesirable microorganism and replacing with a synthetic microorganism further comprises promoting colonization of the synthetic microorganism in the subject. In some embodiments, the promoting colonization of the synthetic microorganism in the subject comprises administering to the subject a promoting agent, optionally where the promoting agent is a nutrient, prebiotic, commensal, stabilizing agent, humectant, and/or probiotic bacterial species. In some embodiments, the promoting comprises administering a probiotic species at from 10⁵ to 10¹⁰ cfu, 10⁶ to 10⁹ cfu, or 10⁷ to 10⁸ cfu to the subject after the initial decolonizing step.

In some embodiments, the nutrient is selected from sodium chloride, lithium chloride, sodium glycerophosphate, phenylethanol, mannitol, tryptone, peptide, and yeast extract. In some embodiments, the prebiotic is selected from the group consisting of short-chain fatty acids (acetic acid, propionic acid, butyric acid, isobutyric acid, valeric acid, isovaleric acid), glycerol, pectin-derived oligosaccharides from agricultural by-products, fructo-oligosaccarides (e.g., inulin-like prebiotics), galacto-oligosaccharides (e.g., raffinose), succinic acid, lactic acid, and mannan-oligosaccharides.

In some embodiments, the probiotic is selected from the group consisting of Bifidobacterium breve, Bifidobacterium bifidum, Bifidobacteriun lactis, Bifidobacterium infantis, Bifidobacterium breve, Bifidobacterium longum, Lactobacillus reuteri, Lactobacillus paracasei, Lactobacillus plantarum, Lactobacillus johnsonii, Lactobacillus rhamnosus, Lactobacillus acidophilus, Lactobacillus salivarius, Lactobacillus casei, Lactobacillus plantarum, Lactococcus lactis, Streptococcus thermophiles, and Enterococcus faecalis.

In some embodiments, the undesirable microorganism is an antimicrobial agent-resistant microorganism. In some embodiments, the antimicrobial agent-resistant microorganism is an antibiotic resistant bacteria. In some embodiments, the antibiotic-resistant bacteria is a Gram-positive bacterial species selected from the group consisting of a Streptococcus spp., Cutibacterium spp., and a Staphylococcus spp. In some embodiments, the Streptococcus spp. is selected from the group consisting of Streptococcus pneumoniae, Streptococcus mutans, Streptococcus sobrinus, Streptococcus pyogenes, and Streptococcus agalactiae. In some embodiments, the Cutibacterium spp. is selected from the group consisting of Cutibacterium acnes subsp. acnes, Cutibacterium acnes subsp. defendens, and Cutibacterium acnes subsp. elongatum. In some embodiments, the Staphylococcus spp. is selected from the group consisting of Staphylococcus aureus, Staphylococcus epidermidis, and Staphylococcus saprophyticus. In some embodiments, the undesirable microorganism is a methicillin-resistant Staphylococcus aureus (MRSA) strain that contains a staphylococcal chromosome cassette (SCCmec types I-III), which encode one (SCCmec type I) or multiple antibiotic resistance genes (SCCmec type II and III), and/or produces a toxin. In some embodiments, the toxin is selected from the group consisting of a Panton-Valentine leucocidin (PVL) toxin, toxic shock syndrome toxin-1 (TSST-1), staphylococcal alpha-hemolysin toxin, staphylococcal beta-hemolysin toxin, staphylococcal gamma-hemolysin toxin, staphylococcal delta-hemolysin toxin, enterotoxin A, enterotoxin B, enterotoxin C, enterotoxin D, enterotoxin E, and a coagulase toxin.

In some embodiments, the subject treated with a method according to the disclosure does not exhibit recurrence or colonization of the undesirable microorganism as evidenced by swabbing the subject at the at least one site for at least two weeks, at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

The disclosure provides a synthetic microorganism for durably replacing an undesirable microorganism in a subject. The synthetic microorganism comprises a molecular modification designed to enhance safety by reducing the risk of systemic infection. In one embodiment, the molecular modification causes a significant reduction in growth or cell death of the synthetic microorganism in response to blood, serum, plasma, or interstitial fluid. The synthetic microorganism may be used in methods and compositions for preventing or reducing recurrence of dermal or mucosal colonization or recolonization of an undesirable microorganism in a subject.

The disclosure provides a synthetic microorganism for use in compositions and methods for treating or preventing, reducing the risk of, or reducing the likelihood of colonization, or recolonization, systemic infection, bacteremia, or endocarditis caused by an undesirable microorganism in a subject.

The disclosure provides a synthetic microorganism comprising a recombinant nucleotide comprising at least one kill switch molecular modification comprising a first cell death gene operatively associated with a first regulatory region comprising an inducible first promoter, wherein the first inducible promoter exhibits conditionally high level gene expression of the recombinant nucleotide in response to exposure to blood, serum, or plasma of at least three fold increase of basal productivity. In some embodiments, the inducible first promoter exhibits, comprises, is derived from, or is selected from a gene that exhibits upregulation of at least 5-fold, at least 10-fold, at least 20-fold, at least 50-fold, or at least 100-fold within at least 30 min, 60 min, 90 min, 120 min, 180 min, 240 min, 300 min, or at least 360 min following exposure to blood, serum, or plasma.

In some embodiments, the synthetic microorganism comprises a kill switch molecular modification comprising a first cell death gene operably linked to a first regulatory region comprising a inducible first promoter, wherein the first promoter is activated (induced) by a change in state in the microorganism environment in contradistinction to the normal physiological (niche) conditions at the at least one site in the subject.

In some embodiments, the synthetic microorganism further comprises an expression clamp molecular modification comprising an antitoxin gene specific for the first cell death gene or a product thereof, wherein the antitoxin gene is operably associated with a second regulatory region comprising a second promoter which is constitutive or active upon dermal or mucosal colonization or in a complete media, but is not induced, induced less than 1.5-fold, or is repressed after exposure to blood, serum or plasma for at least 30 minutes. In some embodiments, the second promoter is active upon dermal or mucosal colonization or in TSB media, but is repressed by at least 2 fold upon exposure to blood, serum or plasma after a period of time of at least 30 min, 60 min, 90 min, 120 min, 180 min, 240 min, 300 min, or at least 360 min.

In some embodiments, the synthetic microorganism exhibits measurable average cell death of at least 50% cfu reduction within at least 1, 5, 15, 30, 60, 90, 120, 180, 240, 300, or 360 minutes following exposure to blood, serum, or plasma. In some embodiments, the synthetic microorganism exhibits measurable average cell death of at least 70%, at least 80%, at least 90%, at least 95%, at least 99%, at least 99.5%, at least 99.8%, or at least 99.9% cfu count reduction within at least 1, 5, 15, 30, 60, 90, 120, 180, 240, 300, or 360 minutes following exposure to blood, serum, or plasma.

In some embodiments, the synthetic microorganism comprises a kill switch molecular modification that reduces or prevents infectious growth of the synthetic microorganism under systemic conditions in a subject.

In some embodiments, the synthetic microorganism comprises at least one molecular modification that is integrated to a chromosome of the synthetic microorganism.

In some embodiments, the synthetic microorganism is derived from a target microorganism having the same genus and species as an undesirable microorganism. In some embodiments, the target microorganism is susceptible to at least one antimicrobial agent. In some embodiments, the target microorganism is selected from a bacterial or yeast target microorganism. In certain embodiments, the target microorganism is capable of colonizing a intramammary, dermal and/or mucosal niche.

In some embodiments, the target microorganism has the ability to biomically integrate with the decolonized host microbiome. In some embodiments, the synthetic microorganism is derived from a target microorganism isolated from the host microbiome.

The target microorganism may be a bacterial species capable of colonizing a dermal and/or mucosal niche and may be a member of a genus selected from the group consisting of Staphylococcus, Streptococcus, Escherichia, Acinetobacter, Bacillus, Mycobacterium, Mycoplasma, Enterococcus, Corynebacterium, Klebsiella, Enterobacter, Trueperella, and Pseudomonas.

The target microorganism may be selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa, optionally wherein the target strain is a Staphylococcus aureus 502a strain or RN4220 strain.

In some embodiments, the synthetic microorganism comprises a kill switch molecular modification comprising a cell death gene selected from the group consisting of sprA1, sprA2, kpn1, sma1, sprG, relF, rsaE, yoeB, mazF, yefM, or lysostaphin toxin gene. In some embodiments, the cell death gene comprises a nucleotide sequence selected from the group consisting of SEQ ID NOs: 122, 124, 125, 126, 127, 128, 274, 275, 284, 286, 288, 290, 315, and 317, or a substantially identical nucleotide sequence.

In some embodiments, the inducible first promoter is a blood, serum, and/or plasma responsive promoter. In some embodiments, the first promoter is upregulated by at least 1.5 fold, at least 3-fold, at least 5-fold, at least 10-fold, at least 20-fold, at least 50-fold, or at least 100-fold within a period of time selected from the group consisting of at least 30 min, 60 min, 90 min, 120 min, 180 min, 240 min, 300 min, and at least 360 min following exposure to human blood, serum or plasma. In some embodiments, the first promoter is not induced, induced less than 1.5 fold, or is repressed in the absence of the change of state. In some embodiments, the first promoter is induced at least 1.5, 2, 3, 4, 5 or at least 6 fold within a period of time in the presence of serum, blood or plasma. In some embodiments, the first promoter is not induced, induced less than 1.5 fold, or repressed under the normal physiological (niche) conditions at the at least one site.

In some embodiments, the inducible first promoter comprises or is derived from a gene selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A). In some embodiments, the inducible first promoter comprises a nucleotide sequence selected from the group consisting of SEQ ID NO: 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical nucleotide sequence thereof.

In some embodiments, the synthetic microorganism comprises an expression clamp molecular modification comprising a second promoter operatively associated with an antitoxin gene that encodes an antisense RNA sequence capable of hybridizing with at least a portion of the first cell death gene. In some embodiments, the antitoxin gene encodes an antisense RNA sequence capable of hybridizing with at least a portion of the first cell death gene. In some embodiments, the antitoxin gene is selected from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively. In some embodiments, the antitoxin gene comprises a nucleotide sequence selected from the group consisting of SEQ ID NOs: 273, 306, 307, 308, 309, 310, 311, 312, 314, 319, 322, 342, 347, 362, 364, 368, 373, 374, 375, 376, 377, and 378, or a substantially identical nucleotide sequence.

In some embodiments, the second promoter comprises or is derived from a gene selected from the group consisting of clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR family transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho. In some embodiments, the second promoter is a P_{clfB} (clumping factor B) that comprises a nucleotide sequence of SEQ ID NO: 117, 118, 129 or 130, or a substantially identical nucleotide sequence thereof.

In some embodiments, the synthetic microorganism comprises a virulence block molecular modification, and/or a nanofactory molecular modification. In some embodiments, the virulence block molecular modification prevents horizontal gene transfer of genetic material from the undesirable microorganism.

In some embodiments, the nanofactory molecular modification comprises an insertion of a gene that encodes, a knock out of a gene that encodes, or a genetic modification of a gene that encodes a product selected from the group consisting of an enzyme, amino acid, metabolic intermediate, and a small molecule.

The disclosure provides a composition comprising an effective amount of a synthetic microorganism according to the disclosure and a pharmaceutically acceptable carrier, diluent, surfactant, emollient, binder, excipient, sealant, barrier teat dip, lubricant, sweetening agent, flavoring agent, wetting agent, preservative, buffer, or absorbent, or a combination thereof. In some embodiments, the composition further comprises a promoting agent. In some embodiments, the promoting agent is selected from a nutrient, prebiotic, sealant, barrier teat dip, commensal, and/or probiotic bacterial species.

The disclosure provides a single dose unit comprising a composition or synthetic microorganism of the disclosure. In some embodiments, the single dose unit comprises at least at least about 10⁵, at least 10⁶, at least 10⁷, at least 10⁸, at least 10⁹, at least 10¹⁰ CFU, or at least 10¹¹ of the synthetic strain and a pharmaceutically acceptable carrier. In some embodiments, the single dose unit is formulated for topical administration. In some embodiments, the single dose unit is formulated for intramammary, dermal or mucosal administration to at least one site of the subject.

The disclosure provides a synthetic microorganism, composition according to the disclosure for use in the manufacture of a medicament for use in a method eliminating, preventing, or reducing the risk of the recurrence of a undesirable microorganism in a subject. In some embodiments, the subject may be a mammalian subject such as a human, bovine, caprine, porcine, ovine, canine, feline, equine or other mammalian subject. In some embodiments, the subject is a bovine subject.

A method is provided for treating and/or preventing mastitis or an intramammary infection in a bovine, ovine, caprine, or porcine subject, comprising (a) decolonizing the subject at at least one site; and (b) recolonizing the subject at the at least one site with a live biotherapeutic composition according to the disclosure. The method may be effective to reduce the somatic cell count (SCC) in milk from the subject within about 1, 2, or 3 weeks following first inoculation when compared to baseline pre-inoculation SCC, optionally wherein the SCC is reduced to no more than 300,000 cells/mL, no more than 200,000 cells/mL, or preferably no more than 150,000 cells/mL.

The at least one site may include one or more of teat canal, teat cistern, gland cistern, streak canal, teat apices, teat skin, udder skin, perineum skin, rectum, vagina, muzzle area, nares, and/or oral cavity of the subject.

The disclosure provides a kit for preventing or reducing recurrence of dermal or mucosal colonization or recolonization of an undesirable microorganism in a subject, the kit comprising in at least one container, comprising a synthetic microorganism, composition, or single dose of the disclosure, and optionally one or more additional components selected from a second container comprising a decolonizing agent, a sheet of instructions, at least a third container comprising a promoting agent, and/or an applicator.

### BRIEF DESCRIPTION OF THE DRAWINGS

FIG. 1A shows an exemplary method for, e.g., up to 6 months protection for mastitis free cows. (a) A cow due for protection is decolonized using, for example, a broad spectrum antiseptic, for example, povidone iodine. (b) After decolonization, the cow is recolonized with a protectant composition of the disclosure comprising live biotherapeutic product. (c) The recolonized cow goes back into production.

FIG. 1B shows a diagram of a representative molecular modification inserted to a Staphylococcus aureus, e.g., BioPlx-01, to create a synthetic microorganism BioPlx strain. A cassette comprising the molecular modification comprises a kill switch and an expression clamp, including expression clamp (e.g., ClfB) promoter cloned to drive expression of the SprA1 antisense (antitoxin) RNA wherein the cassette is incorporated into the same expression module from a kill switch comprising a serum-responsive promoter (e.g., P_{hlgA}) operably associated with SprA1 toxin gene. In this strain, serum/blood exposure activates the toxin (e.g., up to 350-fold or more) but not the antitoxin, and growth in TSB or on the skin activates antitoxin but not toxin.

FIG. 2 shows shuttle vector PCN51 used to clone genes into an E coli-Staphylococcus aureus pass-through strain (IMO8B) for transfection of the vector into BioPlx-01 for evaluation.

FIGS. 3A-3C shows Table 4A with primer sequences for recombinant construction of synthetic Staphylococcus aureus from strain BioPlx-01.

FIGS. 4A-4D shows Table 4B with primer sequences for CRISPR construction of synthetic Staphylococcus aureus from strain BioPlx-01.

FIG. 5A shows a genetic map of a pKOR1 Integrative Plasmid depicting the repF (replication gene of pE194ts), secY570 (N-terminal 570 nucleotides of secY including ribosome binding site), cat (chloramphenicol acetyltransferase), attP (page lambda attachment site), ori(−) (ColE1 plasmid replication origin), and bla (b-lactamase). (+) or (−) indicates functions in gram positive (+) or gram negative (−) bacteria. The Pxyl/tetO promoter and the transcription direction of the promoter are indicated by an arrow.

FIG. 5B shows a genetic map of a pIMAY Integrative Plasmid. (accession number JQ62198).

FIG. 6 shows fold-induction of the HlgA (gamma hemolysin) promoter candidate in a methicillin-susceptible Staphylococcus aureus strain BioPlx-01 by incubation with human serum. Expression was normalized to a housekeeping gene (gyrB) and was compared with that in cells growing logarithmically in liquid TSB media.

FIG. 7 shows fold-induction of the SstA (iron transport) promoter candidate in a methicillin-susceptible Staphylococcus aureus strain BioPlx-01 by incubation with human serum. Expression was normalized to a housekeeping gene (GyrB) and was compared with that in cells growing logarithmically in liquid TSB media.

FIG. 8 shows CRISPR gRNA target site intergenic region identified between 1,102,100 and 1,102,700 bp in the Staphylococcus aureus 502a genome, GenBank: CP007454.1.

FIG. 9 shows a representative screen shot of CRISPRScan used to find putative gRNAs for use in CRISPR methods.

FIG. 10 shows cassette for integration via CRISPR and layout of the pCasSA vector. Cap1A is a constitutive promoter controlling gRNA transcription. Target seq is targeting sequence, for example, with 10 possible cutting targets (1.1, 1.2 etc.). sgRNA is single-strand guide RNA (provides structural component). Xbal and Xhol are two restriction sites used to add the HA's to the pCasSA vector. HAs are homologous arms to use as templates for homology directed repair (typically 200-1000 bp). P_{rpsL}-mCherry is a constitutive promoter controlling the “optimized” mCherry. P_{rpsL}-Cas9 is a constitutive promoter controlling Cas9 protein expression.

FIG. 11 shows vectors for use in the present disclosure. A is a vector used for promoter screen with fluorescence using pCN51. B is a vector for promoter screen with cell death gene. C is a vector for chromosomal integration using CRISPR. D is a vector for chromosomal integration using homologous recombination. Left & Right (or upstream and downstream) HA: homology arms to genomic target locus, CRISPR targeting: RNA guide to genomic locus, mCherry: fluorescent reporter protein, Cas9 protein: CRISPR endonuclease, kanR: kanamycin resistance, oriT: origin of transfer (for integration), and sma1: representative kill gene (restriction endonuclease).

FIG. 12A-12C shows nucleotide sequence (SEQ ID NO: 131) of pIMAY Integrative Plasmid. (accession number JQ62198).

FIG. 13A shows activity of promoter candidates isdA, isdB, hlgA2, hrtAB, isdG, sbnE, lrgA, lrgB, fhuA, fhuB, ear, hlb, splF, splD, dps, and SAUSA300_2617 at 1 min, 15 min and 45 min in serum and fold changes in gene expression vs. media by qPCR.

FIG. 13B shows activity of promoter candidates isdA, isdB, hlgA2, hrtAB, isdG, sbnE, lrgA, lrgB, fhuA, fhuB, ear, hlb, splF, splD, dps, and SAUSA300_2617 at 1 min, 15 min and 45 min in blood and fold changes in gene expression vs. media by qPCR.

FIG. 14 shows inducible inhibition of cell growth of synthetic microorganism pTK1 cells comprising a cell death toxin gene (sprA1) behind a cadmium promoter on a pCN51 plasmid (pTK1) which had been transformed into Staphylococcus aureus RN4220 cells. OD (630 nm) read at 2 hrs post induction. Wild-type 4220 cells showed good cell growth both in the absence of cadmium and in the presence of 500 nM and 1 uM cadmium. pTK1-1 and pTK1-2 cells showed good growth in the absence of cadmium, but cell growth was significantly inhibited in presence of 500 nM and 1 uM cadmium at 2 hours post induction.

FIG. 15A shows a plasmid map of p174 (pRAB11_Ptet-sprA1) zoomed view of the region of the plasmid containing the Ptet-sprA cassette.

FIG. 15B shows the p174 (pRAB11_Ptet-sprA1) whole plasmid in its native circular form.

FIG. 15C shows photographs of plate dilutions at 6 hours synthetic microorganism Staphylococcus aureus 502a p174 cells comprising a cell death toxin gene (sprA1) behind an anhydrotetracycline promoter on a pRAB11-2 plasmid (p174) which had been transformed into Staphylococcus aureus 502a cells. The p174 plasmid containing a deleted spra1 antisense (Das). Plate dilutions at 10e−5 are shown after 6 hours of induction for uninduced (left) and induced (right) 502a p174 (tet-spra1Das) cells on BHI chlor10. The plate on the left (Uninduced) was uncountable at 10e−5 but at 10e−6 counted ˜720 colonies. The induced plate on the right at 10e−5 produced 16 colonies. The survival percentage of induced cells at 6 hours post induction was 0.22%.

FIG. 16 shows cell growth pre- and post-induction of four synthetic strains derived from Staphylococcus aureus 502a having a plasmid based inducible expression system comprising four different cell death gene candidates sprA1, 187-lysK, Holin, and sprG. The candidate cell death genes had been cloned behind an tetracycline inducible promoter on pRAB11 plasmids and transformed into Staphylococcus aureus 502a cells. Calculated OD600 readings were taken at T=0, 30, 60, 120, and 240 min after induction of AtC induced (+) strains illustrated by dashed lines (- - - - - -) and uninduced (−) strains indicated by solid lines (———) for BP_068 (502a pRAB11-Ptet-sprA1), BP_069 (502a pRAB11-Ptet-187lysK), BP_070 (502a pRAB11-Ptet-holin), and BP_071 (502a pRAB11-Ptet-sprG1) and compared to BP_001 (502a wt) in BHI media. Each of the induced (+) strains BP_068 (sprA1), BP_069 (187lysK) and BP_070 (holin) exhibited both (i) good cell growth pre-induction and (ii) significant inhibition of cell growth post-induction. BP_068 (+) exhibited the best inhibition of cell growth at each time point T=30, T=60, T=60, T=120 and T=240 min post-induction, so the sprA1 gene was selected for initial further development of a kill switch in Staphylococcus aureus 502a.

FIG. 17 shows a bar graph showing difference in the colony forming units (cfu)/mL between T=0 (gray) and 240 min(black) of un-induced (−) and anhydrotetracycline induced (+) strains BP_068 (502a pRAB11-Ptet-sprA1), BP_069 (502a pRAB11-Ptet-187lysK), BP_070 (502a pRAB11-Ptet-holin), and BP_071 (502a pRAB11-Ptet-sprG1) compared to BP_001 (502a wt) in BHI media. Each of the induced (+) strains BP_068 (sprA1), BP_069 (187lysK) and BP_070 (holin) exhibited both (i) good cell growth pre-induction and (ii) significant inhibition of cell growth post-induction. BP_068 exhibited the best inhibition of cell growth 240 min post-induction, so the sprA1 gene was selected for initial further development of a kill switch in Staphylococcus aureus 502a.

FIG. 18 shows GFP expression fold change of induced (+) and uninduced (−) subcultures of Staphylococcus aureus strains BP_001, BP_055 and BP_076.

FIG. 19 shows a map of the genome for Strain BP_076 (SA 502a, ΔsprA1::Ptet-GFP).

FIG. 20 shows a map of plasmid constructed for making genomic integration in Staphylococcus aureus.

FIG. 21 shows a map of PsbnA-sprA1 kill switch in Staphylococcus aureus 502a genome. Serum and blood responsive promoter PisdB is operably linked to sprA1 toxin cell death gene.

FIG. 22 shows a map of a kill switch construction using serum and blood responsive promoter PisdB operably linked to sprA1 toxin cell death gene and an expression clamp comprising a second promoter clfB operably linked to sprA AS to prevent leaky expression of the toxin in the absence of blood or serum. The kill switch is incorporated to the Staphylococcus aureus 502a genome.

FIG. 23 shows a growth curve of three strains when exposed to human serum compared to TSB: 502a—Staphylococcus aureus wild type, Staphylococcus aureus BP_011-502a ΔsprA1-sprA1(AS), and Staphylococcus aureus BP_084-502a ΔPsprA::PsbnA in which the kill switch is integrated to the genome of Staphylococcus aureus 502a. The dashed lines represent the strains grown in serum, and the solid lines represent the strains grown in TSB. After 180 minutes, the strain BP_084 with the integrated kill switch shows a growth curve that is significantly reduced compared to the wild type in serum and the kill switch in complex media. After 3 hours of exposure to human serum, the Staphylococcus aureus BP_084 (502a ΔPsprA::PsbnA) cells exhibited 98.84% measurable average cell death compared to the same BP_084 cells in TSB.

FIG. 24 shows a graph of change in mean cell counts over 24 hours in TSB and human serum for unmodified wild-type Staphylococcus aureus strain 502a and kill-switched S. aureus strain BP_088 (“BP88”) on 502a base strain. At t=0 hours, 502a and BP88 were at mean cell count of about 1×10⁵ cells in TSB and serum. After 6 hours, mean cell counts for wild-type 502a in TSB and serum were 2×10⁸ and 2×10⁷ cells, respectively. In contrast, after 6 hours, mean cell counts for BP88 in TSB was 1×10⁸, while mean cell count in serum dropped to no detectable cells, and remained at no detectable cells over the 24 hour assay. This assay demonstrates that kill switched cells kill themselves in blood, serum, and plasma. They can colonize in the absence of blood serum or plasma, but cannot infect.

FIG. 25 shows a partial sequence alignment of the insertion sequences to target strain Staphylococcus aureus BP_001 (502a) comprising isdB::sprA1 in three synthetic strains. The serum inducible promoter is isdB. The toxin gene is sprA1. Sequence A is the mutation free sequence for BP_118, sequence B is the frame shifted mutant which shows how the isdB reading frame is impacted for BP_088, and sequence C contains two extra STOP codons after isdB in different frames for BP_115 (triple stop).

FIG. 26 shows a graph of growth curves for synthetic S. aureus strain BP_088 isdB::sprA1 in human serum (dashed lines) or tryptic soy broth (TSB) complete media (solid lines) in colony forming units per mL (cfu/mL) of culture over time (8 hours)(n=3, each condition). BP_088 growth in TSB increased from about 1×10⁷ to about 1×10⁹ cfu/ml over 4 hrs. In contrast, BP_088 exhibited significantly decreased growth in human serum from about 1×10⁷ to about 1×10³ cfu/ml over 2 hrs or less. BP_088 was unable to grow when exposed to serum, despite frame shift in isdB gene extending the reading frame by 30 bp or 10 amino acids.

FIG. 27 shows a graph of growth curves for synthetic S. aureus strain BP_115 isdB::sprA1 (n=3) and target strain wt 502a (BP_001) in human serum (dashed lines) or TSB (solid lines) in cfu/mL of culture over time (8 hours). BP_115 and wt 502a growth in TSB increased from about 1×10⁷ to about 1×10⁹ cfu/ml over about 4-6 hrs. In serum, wt 502a growth increased from about 1×10⁷ to about 6×10⁷ over about 6 hrs. In contrast, BP_115 exhibited significantly decreased growth in human serum from about 1×10⁷ to about 1×10³ cfu/ml over 2 hrs or less. Parent target strain wt 502a was able to grow when exposed to serum, but S. aureus synthetic strain BP_115 with isdB::sprA1 was unable to grow when exposed to serum.

FIG. 28 shows a graph of growth curves for BP_118 (n=3) and BP_001 (wt 502a) (n=1) in human serum and TSB. Both BP_0118 and wt502a exhibit increased growth in TSB over 8 hr. wt502a exhibits some increased growth in human serum over 8 hr. However, BP_0118 exhibits significantly decreased growth over 2 hrs or less in human serum

FIG. 29 shows a graph of average CFU/mL for S. aureus synthetic strains BP_088, BP_115, and BP_118 in TSB vs. human serum. Each of the strains is able to grow in TSB over 2-8 hr. Each of the strains exhibits significantly decreased growth when exposed to human serum for 2 hrs or less.

FIG. 30 shows multiple synthetic strains of Staphylococcus aureus and E. coli with plasmid identifiers, action genes, insertion DNA sequences, target sites for genome insertion, DNA sequences of upstream and downstream homology arms, and generated strain designations.

FIG. 31 shows a graph of induced and uninduced growth curves for the E. coli strain IM08B (BPEC_023) harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Within 2 hours of induction, the BPEC_023 E. coli culture growth rate slowed significantly for each following time point.

FIG. 32 shows a graph of the growth curves for the Staph aureus strain BP_001 harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Overexpression of the truncated sprA1 gene BP_DNA_090 (SEQ ID NO: 47) (encoding BP_AA_014 (SEQ ID NO: 84) had an effect on the growing E. coli and Staph aureus cultures. The growth curves for the uninduced cultures began diverging from the induced cultures within 2 hrs following the addition of ATc, where the uninduced cultures continued to grow in log phase and the growth of the induced cultures slowed dramatically directly after the addition of ATc.

FIG. 33 shows a graph of the average (n=6) of viable CFU/mL of Staph aureus synthetic strain BP_088 (0 and 500 generation strains) when grown in human serum (dashed lines) or TSB (solid lines). BP_001 (n=6) in TSB and serum was plotted as a wild type control. Error bars represent one standard deviation of all six replicates. The BP_088-500 generation sample is represented by solid squares (▪) and the 0 generation sample (▴). Parent strain BP_001 is represented by a solid circle. Synthetic strain BP_088 exhibits functional stability over at least 500 generations as evidenced by its retained inability to grow when exposed to human serum compared to BP_088 at 0 generations. After 2 hrs in human serum, BP_088 exhibited significantly decreased cfu/mL by about 4 orders of magnitude even after about 500 generations.

FIG. 34 shows a photograph of an Agarose gel for PCR confirmation of isdb::sprA1 in BP_118 showing the PCR products of from the secondary recombination PCR screen with primers DR_534 and DR_254. Primer DR_534 binds to the genome outside of the homology arm, and the primer DR_254 binds to the sprA1 gene making size of the amplicon is 1367 bp for s strain with the integration and making no PCR fragment if the integration is not present. BP_001 was run as a negative control to show the integration is not present in the parent strain.

FIG. 35 shows a map of the genome of Staph aureus synthetic BP_118 where the sprA1 gene was inserted. The map was created with the Benchling program.

FIG. 36 shows a graph of Staph aureus synthetic strain BP_118 and parent target strain BP_001 in kill switch assay in TSB or human serum over 4 hrs. The points plotted on the graph represent an average of 3 biological replicates and the error bars represent the standard deviation for triplicate samples. The solid lines represent the cultures grown in TSB and the dashed lines represent cultures grown in human serum. The human serum assay suggested the kill switch was effective with dramatic reduction in viable cfu/mL for strain BP_118 in serum with no difference in growth in complex media (TSB) compared to the parent strain BP_001.

FIG. 37 shows a graph of an assay of the average CFU/mL for BP_112 (ΔsprA1-sprA1(AS), Site_2::PgyrB-sprA1(AS)(long), isdB::sprA1)(n=3) and BP_001 (n=1) when they are grown in serum (dashed lines) and TSB (solid lines) over an 8-hour period. The error bars represent the standard deviation of the averaged values. The human serum assay suggested kill switch was effective with dramatic reduction in viable CFU/mL for strain BP_112, with no difference in growth in complex media (TSB) compared to the wild-type parent strain BP_001

FIG. 38 shows a bar graph of the fold change in expression of 25 genes from Staph aureus at 30 and 90 minute time points in TSB and human serum. The number of reads for each gene was converted to transcripts per million (TPM), the replicates were averaged for each condition (n=3), normalized to the expression of the housekeeping gene gyrB, subtracted from the initial expression levels at t=0, and sorted for the most differentially expressed between the two media conditions at the 90 minute time point. The gene on the bottom of the chart (CH52_00245) had a value of 175 fold upregulation, but was cut short on this figure in order to enlarge the chart and maximize the clarity of the rest of the data.

FIG. 39 shows a graph of kill switch activity over 4 hours as average CFU/mL of 4 Staph aureus synthetic strains with different kill switch integrations in human serum compared to parent target strain BP_001. Strains BP_118 (isdB::spra1), BP_092 (PsbnA::sprA1) and BP_128 (harA::sprA1) each exhibited a decrease in CFU/mL at both the 2 and 4 hour time points. BP_118 (isdB::spra1) exhibited strongest kill switch activity as largest decrease in CFU/mL.

FIG. 40 shows a bar graph of the concentration of cfu/mL for all of the strains tested human plasma or TSB, at both t=0 and after 3.5 hours of growth (t=3.5). The viable cfu/mL of strains BP_088, BP_101, BP_108, and BP_109 showed over a 99% reduction after 3.5 hours in human plasma. BP_092 showed a 95% reduction in viable cfu/mL after 3.5 hours in human plasma. BP_001 showed very little difference in viable cfu/mL after 3.5 hours in human plasma. All strains grew in TSB media.

FIG. 41 shows a graph of the growth curves as OD600 values of four synthetic E. coli (sprA1) strains 1, 2, 15, 16 grown for 5 hrs in LB (+/−ATc) and induced at t=1 hr. Two different types of target E. coli strains were employed: BPEC_006 strains 1, 2, and 15 are from E. coli K12-type target strain IM08B, and strain 16 is from the bovine E. coli target strain obtained from Udder Health Systems. All induced strains (dashed lines) showed significant decrease in growth over 2-5 hr time points.

FIG. 42 shows a graph of the growth curves as OD600 values over 5 hrs with of (4) different synthetic E. coli isolates grown in LB with an inducible hokB or hokD gene integrated in the genome of K12-type E. coli target strain IM08B. Samples were induced by adding ATc to the culture 1 h post inoculation. The dashed line represents the cultures that were spiked with ATc to induce expression of the putative toxin genes and the solid line represents cultures that did not get induced by ATc. The hokD sample exhibited a diverging curve between the induced and uninduced samples. The hokB_1 is the bovine E. coli strain from Udder Health Systems and the spiked and unspiked samples grew much faster than the other 3 strains tested here

FIG. 43 shows a graph of the average (n=3) growth curves as OD600 values over 5 hrs of two synthetic E. coli strains with relE or yafQ gene integrated in the genome (n=3) grown in LB (+/−ATc). The dashed lines represent the cultures that were spiked with ATc to induce expression of the putative toxin genes and the solid lines represent cultures that did not get induced by ATc. The error bars represent one standard deviation for the averaged OD600 values for each strain. The relE gene showed diverging curves between the cultures that were induced and the uninduced cultures, where the induced cultures had significantly lower OD600 readings. The induced yafQ cultures showed a slightly slower growth between hours 2 and 4 than the uninduced cultures, but at 5 hours the two groups had nearly identical OD600 values.

FIG. 44 shows a graph the concentrations of synthetic S. aureus BP_109 and BP_121 cells grown in in TSB and human synovial fluid over the course of a 4 hour growth assay. Both BP_121 (control) and BP_109 (kill switch) cultures grew in TSB. BP_109 showed a rapid decrease in viable cfu/mL in the synovial fluid condition.

FIG. 45 shows a graph of the concentration of synthetic Staph aureus BP_109 (kill switch) and BP_121 (control) cells in TSB and Serum Enriched CSF over the course of a 6 hour assay. Both BP_121 (control) and BP_109 (kill switch) cultures grew in TSB. BP_121 also grew in CSF enriched with 2.5% human serum; however, BP_109 showed a rapid decrease in cfu/mL in the CSF condition.

FIG. 46 shows a graph of an in vivo bacteremia study in mice after tail vein injection of 10\hat{ }7 wild-type Staphylococcus aureus strains BP_001 killed (2), BP_001 WT (3), CX_001 WT(5) or synthetic Staphylococcus aureus strains comprising a kill switch BP109(4), CX_013 (6) showing avg. health, body weight, and survival over 7 days. Groups receiving BP_001 WT (3) and CX_001 WT (5) exhibited adverse clinical observations starting at day 1, greater than 15% reduction in avg body weight and death starting at day 2. By day 7, all 5 mice in CX_001 WT (5) group had died and 3 of 5 mice in BP_001 WT (3) group had died as shown at the bottom of chart. In contrast, mice receiving synthetic kill switch strains BP109 (4) and CX_013 (6), and BP_001 killed (2) all survived and exhibited no more than 10% weight loss compared to initial weight.

FIG. 47 shows a graph of animal health in an in vivo SSTI mouse study as measured by abscess formation, or lack thereof, following single SC injection of 10\hat{ }7 synthetic Staph aureus KS microorganisms or wild type Staph aureus parent strains over 10 days. Mice in KS Groups 4 (BP_109, n=5) and 6 (CX_013, n=5), respectively, maintained health over the course of this study, as compared to abscess formation present in about half of the wild type parent strains Group 3 (BP_001, n=5) and Group 6 (CX_013, n=5), respectively. Animals in the negative control Groups 1 (vehicle, n=5) and 2 (killed WT BP_001, n=5) all remained healthy throughout the study and are not shown.

FIG. 48 shows a graph of OD600 growth curves over 3 hours for Streptococcus agalactiae (BPST_002) transformed with plasmids p174 (sprA1) or p229 (GFP). The starting cultures were inoculated at a 1:10 dilution from stationary phase cultures. The t=0 hr OD was taken before ATc induction. The dashed line represents the cultures that were induced with ATc and the solid line represents control cultures. All data points represent single cultures. Overexpression of sprA1 toxin gene was able to inhibit S. agalactiae cell growth in exponential phase.

FIG. 49 shows a bar graph of fluorescence values at 3 hours after induction of Streptococcus agalactiae (BPST_002) transformed with plasmid p229 (GFP). The starting cultures were inoculated at a 1:10 dilution from stationary phase cultures. Cultures were grown in duplicate and fluorescence readings were performed in triplicate. Significantly increased fluorescent values of induced p229 cultures indicate the ability of the P_{XYL/Tet} promoter system of pRAB11 to function as an ATc inducible promoter in S. agalactiae.

FIG. 50 shows a bar graph calculated from the CFU/mL data of Stability Suspension D containing BP_123, BPST_002, BPEC_006 at 0 and 24 hours. All dilutions were plated in duplicate on TSB plates. CFU/mL data was calculated from the 10⁻⁴ dilution. The observed CFU/mL at t=0 and 24 h supports the stability of cell suspensions containing a mixture of S. aureus, S. agalactiae and E. coli.

### DETAILED DESCRIPTION

Mastitis, commonly due to intramammary infection (IMI), occurs in dairy herds globally. Often requiring antibiotic intervention, it is a burden both to the wellbeing of the animal and the economic output of the herd through a reduction in milk yield, withholding of milk from antibiotic-treated cows, and culling of animals in severe cases. Murphy et al., 2019, Scientific Reports 9: Article 6134.

Keratine is a mesh-like substance that partially occludes the teat canal lumen and inhibits bacterial penetration. Smooth muscle around the teat canal maintains tight closure and inhibits bacterial penetration. Many leukocytes, or white blood cells, kill bacteria or process bacteria by presenting them to lymphocytes for antibody production. In the face of clinical or subclinical infections leukocytes nigrate to the udder from the blood.

Cows must calve to produce milk and the lactation cycle is the period between one calving and the next. The cycle is split into four phases, the early, mid and late lactation (each of about 120 days, or d) and the dry period (which may last as long as 65 d). In an ideal world, cows calve about every 12 months.

Bacterial strains commonly associated with mastitis and intramammary infection include Staphylococcus aureus, coagulase-negative staphylococcus, Escherichia coli, Streptococcus uberis, and Streptococcus dysgalactiae. These bacterial strains may be treated using a broad-spectrum antibiotic, for example, by intramammary infusion using a cephalosporin, such as ToDAY® cephapirin sodium, Boehringer Ingelheim Vetmedica, Inc., or SPECTRAMAST® DC ceftiofur hydrochloride, Zoetis. However, problems with use of a broad-spectrum antibiotic include development of resistant strains and milk contamination with antibiotics.

Mastitis appears in two forms: either clinical, characterized by visible symptoms, sometimes general illness, and a long lasting negative effect on milk production, or subclinical, without visible symptoms but with an increase in somatic cell count (SCC) and suboptimal milk production. Vanderhaeghen et al., 2014; J Dairy Sci. 97:5275-5293.

Mastitis milk culture results may reveal infection with contagious pathogens or environmental pathogens. Contagious pathogens may occur from the handler, other infected animals or milk of other infected animals. Attempts to minimize these infections may include proper milking hygiene including post milking teat disinfection, milking infected animals last, and effective herd management. Contagious pathogens include Gram-positive Streptococcus agalactiae and Streptococcus uberis. Gram-positive, Coagulase-positive pathogens include Staphylococcus aureus. Other contagious pathogens include Mycoplasma spp. and Prototheca spp. Infection from environmental pathogens occurs from bacteria entering the teat end from dirt, manure bedding, milking machines, and human handlers. Attempts to minimize these infections may include proper hygiene, milk machine maintenance, and pre-milking teat disinfection. Environmental pathogens may include Streptococcus (Gram-positive cocci) include Aerococcus spp., such as Aerococcus viridans, Enterococcus spp such as Enterococcus casseliflavus, Enterococcus faecalis, Enterococcus hitae, Enterococcus saccarolyticus, Lactococcus gravieae, Lactococcus lactis, Micrococcus spp, Streptococcus spp, such as Streptococcus bovis, Streptococcus dysgalactiae, Streptococcus equi, Streptococcus vestibularis, other Gram-positive pathogens such as Trueperella pyogenes, Corynebacterium spp., Bacillus spp, Listeria monocytogenes, Gram-positive, coagulase negative cocci including Staphylococcus chromogenes, Staphylococcus saprophyticus, Staphylococcus simulans, and Staphylococcus xylosus, Gram-negative pathogens including Acinetobacter spp such as Acinetobacter baumannii, Aeromonas spp., Citrobacter spp., Enterobacter spp such as Enterobacter amnigenus, Escherichia coli, Flavimonas spp., Hafnia spp., Klebsiella spp. such as Klebsiells oxytoca, Klebsiella pneumonia, Pantoa spp., Plesimonas shigelloides, Proteus spp., Pseudomonas spp. such as Pseudomonas fulva, Salmonella spp., Serrati spp., Serratia marcescens, Stenotrophomonas spp., Yersinia spp. Yeast pathogens include Norcardia spp. and Prototheca spp. In milk, pathogens may be reported semi-quantitatively to assist in understanding the levels at which the pathogen was detected in the milk sample. +1—very few, +2—few, +3—moderate, +4—numerous. Milk stored improperly, such as at room temperature for extended periods will allow for growth of pathogens which may change the semi-quantitation of that pathogen. Wisconsin Veterinary Diagnostic Laboratory, University of Wisconsin-Madison, Interpretation of Mastitis Milk Culture Results, Jul. 15, 2016.

In bovine mastitis, pathogens of high prevalence may include bacterial and yeast pathogens. Bacterial pathogens of high prevalence may include a member of a genus including Staphylococcus, Streptococcus, Escherichia, Bacillus, Mycobacterium, Mycoplasma, Enterococcus, Corynebacterium, Klebsiella, Enterobacter, Trueperella, and/or Pseudomonas.

Bacterial pathogens may include coagulase-positive and/or coagulase-negative staphylococci, for example, coagulase-positive staphylococcus such as Staphylococcus aureus or coagulase-negative staphylococcus species (CNS). The CNS species that have been most frequently identified include S. epidermidis, S. chromogenes, S. simulans, S. saprophyticus, S. haemolyticus, and S. xylosus. Vanderhaeghen et al., 2014; J Dairy Sci. 97:5275-5293. Another common strain is Staphylococcus hyicus, which may be coagulase-variable depending on the strain. A major CNS species found in both goats and sheep is Staphylococcus caprae.

Bacterial pathogens may also include Streptococci spp. The Streptococci spp. may be a Group A, Group B, or Group C/G Step species. The Group A may be Streptococcus pyogenes. The Group B step may be Streptococcus agalactiae. The Group C/G may be Streptococcus dysgalactiae. The bacterial pathogen may be Streptococcus uberis.

Bacterial pathogens may include Bacillus spp. such as Bacillus cereus or Bacillus hemolysis.

Bacterial pathogens may include Mycobacterium spp., for example, Mycobacterium tuberculosis or Mycobacterium bovis.

Bacterial pathogens may include Mycoplasma spp., for example, Mycoplasma bovis.

Bacterial pathogens may include Enterococcus spp. such as Enterococcus faecalis or Enterococcus faecium.

Bacterial pathogens may include Corynebacterium spp., for example, Corynebacterium bovis, Corynebacterium amycolatum, and Corynebacterium ulcerans.

Bacterial pathogens may include Coliforms, for example, Escherichia spp., Klebsiella spp., and Enterobacter spp. Escherichia coli spp. may include, for example, Mammary Pathogenic E. coli (MPEC). Klebsiella spp. may include, for example, Klebsiella pneumonia or Klebsiella oxytoca. Enterobacter spp. may include Enterobacter aerogenes.

Bacterial pathogens may include Trueperella spp. or Arcanobacterium spp., for example, Trueperella pyogenes or Arcanobacterium pyogenes.

Bacterial pathogens may include Pseudomonas spp., for example, Pseudomonas aeruginosa.

Yeast pathogens may include a member of a genus including Candida spp. and/or Cryptococcus spp. Candida spp. pathogens may include Candida parapsilosis, Candida krusei, Candida tropicalis, Candida albicans, and/or Candida glabrata. Cryptococcus pathogens may include Cryptococcus neoformans or Cryptococcus gattii.

Staphylococcus aureus is a coagulase-positive Staphylococcus, which is a general name for a class of bacteria that are small, round, and Gram-positive. Staph. aureus is a contagious pathogen, which is transmitted from infected glands or teats during the milking process. It is a major cause of chronic or recurring clinical mastitis in dairy cows and is believed to be the most significant contagious mastitis pathogen.

Staph. aureus is a commensal organism of the skin and mucosa, and is also found in the environment. Infected cows, either purchased or chronically infected, are the major source for new infections. Heifers with persistently colonized udder or teat skin, muzzles, and vaginas are the primary reservoir. Fresh heifers with colonized body sites can be a source of Staph. aureus when they are introduced into the herd. Chapped, damaged, or broken skin greatly increases the likelihood of Staph. aureus infections. The primary mode of transmission is cow-to-cow during milking, particularly if poor hygiene is a factor and if milking gloves are not worn. Flies have also been implicated in the transmission of Staph. aureus. Infections may increase with age and days of milking. Wisconsin Veterinary Diagnostic Laboratory, University of Wisconsin-Madison, Staphylococcus Aureus, Bulletin 2016.

Staph. aureus infections are typically chronic and subclinical with periodic, recurring mild or moderate clinical signs. There is a positive correlation between bacterial count and somatic cell count (SCC), when Streptococcus agalactiae is not present, but changes in the SCC may be intermittent as bacteria are shed variably and often in low numbers. Chronically infected cows will have an increased SCC and decreased milk production. Staph. aureus may cause gangrenous mastitis that can kill the animal. Abscess formation and tissue damage can occur in chronically infected cows, and abscess breakage can cause reinfection. If abscesses and scar tissue form, permanent damage may occur, reducing milk production and hampering antimicrobial treatment.

The expected cure rate for Staph. aureus infections during lactation is only about 20%. Higher cure rates can be expected in younger animals with only one quarter infected and with a lower SCC at the time of infection. These animals are not likely to be chronically infected. Extended antimicrobial therapy or combination antimicrobial therapy may increase success rates to 30%, but all cow factors should be considered when attempting treatment. Dry cow therapy may also improve success rates. Wisconsin Veterinary Diagnostic Laboratory, University of Wisconsin-Madison, Staphylococcus Aureus, Bulletin 2016.

Known treatment options for Staph. aureus infections can be difficult and animals should be identified for their likelihood of cure. Identifying and eliminating cows through strategic treatment or culling is important for controlling disease. Using herd records to isolate cows with high SCCs or recurrent clinical mastitis is necessary to target infected cows for testing. Herds with greater than 50% of positive milk cultures would indicate a significant problem. It is more common for herds to have less than 30% of milk samples that are positive for Staph. aureus. Cows that have an SCC of greater than 400,000, but test negative for Staph. aureus should be retested within 2-4 weeks due to sporadic shedding of the bacterium. Frequent samples provide a better idea of the infection rate. Wisconsin Veterinary Diagnostic Laboratory, University of Wisconsin-Madison, Staphylococcus Aureus, Bulletin 2016.

Prevention via a good, long-term Staph. aureus management program may be more successful than antimicrobial therapy. Mastitis vaccination programs are currently not effective against Staph. aureus infections. Staph. aureus infections are caused by humans in many cases, which is why excellent pre- and post-milking teat sanitation, milking hygiene including wearing gloves, using single-use towels, and maintaining milking equipment are necessary for reducing transmission of pathogens. All cows should be segregated and a plan for housing and milking should be developed. Purchasing animals should be avoided until prevention practices are in place, and any purchased animals should be tested for contagious pathogens and quarantined until tests are performed. As a screening tool, regular bulk tank cultures are valuable, and mastitis milk cultures for those who do not respond to therapy is necessary. Wisconsin Veterinary Diagnostic Laboratory, University of Wisconsin-Madison, Staphylococcus Aureus, Bulletin 2016. Clearly, alternative approaches to prevention and treatment of mastitis and intramammary infection are desirable.

Persistent IMI is a major issue related to staphylococcal mastitis. It refers to the occurrence of the same infectious agent in the milk throughout a certain period, such as the dry period or part of or even the entire lactation. However, assessing persistence of IMI especially may require consistent strain identification. For example, when an udder quarter yields a series of samples positive for a certain Staphylococcus species over time, it is likely to be persistently infected. Vanderhaeghen et al., 2014; J Dairy Sci. 97:5275-5293.

Another problem with certain S. aureus cell lines is the possibility of intracellular bacterial survival, which may lead to persistent infection. Murphy et al., 2019 Nature Scientific Reports, vol. 9, 6134. Murphy et al. isolated various S. aureus strains from cows having clinical mastitis by bacteriological culture. MAC-T cells, a bovine mammary epithelial cell line was derived from a lactating Holstein cow. Murphy et al demonstrated that strain interaction with bovine mammary epithelial cells and neutrophils varies according to bacterial genotype. Differences in bMEC interaction and bacterial survival between strains indicate that each S. aureus strain had a unique set of characteristics that may determine the outcome of infection in vivo.

Coliform bacteria are also a frequent cause of bovine mastitis. Escherichia coli is the most common coliform bacteria isolated in more than 80% of cases of coliform mastitis. Klebsiella spp. are also common. Suojala et al., 2013, J Vet Pharmacol Therap, doi: 10.1111/jvp.12057. Lipopolysaccharide (LPS), a component of the cell wall of Gram-negative bacteria, is considered to be the primary virulence factor in coliform bacteria. Release of LPS from gram-negative bacteria after a rapid kill by bactericidal antimicrobials has been considered a risk in humans, but has not been demonstrated in association with treatment for bovine E. coli mastitis. In fact, in vivo bactericidal activity has been suggested to be preferable for the treatment of mastitis because of the impaired phagocytosis in the mammary gland. Suojala et al., 2013.

Systemic administration of antimicrobials may be recommended in severe cases of bovine mastitis because of risk of developing bacteremia. Suggested broad-spectrum antimicrobials include trimethoprim-sulfonamides, oxytetracycline, fluoroquinolones, cefquinome, and ceftiofur.

Antimicrobials for which there is some beneficial evidence for effect of treatment for E. coli mastitis include fluoroquinolones and cephalosporins. Fluoroquinolones (enrofloxacin, danofloxacin, and marbofloxacin) are available for treating lactating dairy cattle in some or all EU member states and are authorized and used for the treatment of coliform mastitis. Their action against gram negative agents is bactericidal and concentration dependent. However, in the USA and Australia, systemic administration of fluoroquinolones for mastitis in dairy cows is not approved. Suojala et al., 2013. One problem with use of antimicrobials in treatment of mastitis may be the presence of antimicrobials in milk following systemic administration.

Another problem with use of antimicrobials is development of antimicrobial resistance. For example, Escherichia coli isolates from mastitis have developed resistance to antimicrobials commonly used for years in dairies, including ampicillin, streptomycin, sulfonamides, and oxytetracycline.

In E. coli mastitis with mild to moderate clinical signs, a non-antimicrobial approach (anti-inflammatory treatment, frequent milking and fluid therapy) should be the first option. In cases of severe E. coli mastitis, parenteral administration of fluoroquinolones, or third- or fourth-generation cephalosporins, is recommended due to the risk of unlimited growth of bacteria in the mammary gland and ensuing bacteremia. Evidence for the efficacy of intramammary-administered antimicrobial treatment for E. coli mastitis is limited. Nonsteroidal anti-inflammatory drugs have documented the efficacy in the treatment for E. coli mastitis and are recommended for supportive treatment for clinical mastitis. Suojala et al., 2013.

Streptococcus spp. is a major cause of mastitis, including subclinical mastitis. S. uberis, S. agalactiae, S. dysgalactyiae, S. epidemicus, S. bovis, S. equinus are strains associated with mastitis. Streptococcus strains may be subjected to serological grouping with a commercial latex agglutination kit for identification of streptococcal groups A, B, C, D, F, and G. Control of Streptococci infection involves environmental control including maintenance of a clean dry environment for cows and proper milking procedures. Proper milking procedures include forestripping in all four quarters, use of FDA-approved pre-milking teat disinfectant, for at least 30 seconds, prior to removal with a paper towel or single-use clean and dry cloth towel, post-milking teat disinfectant, and use of barrier teat dip.

Streptococcus uberis is known worldwide as an environmental pathogen responsible for clinical and subclinical mastitis in lactating cows. Streptococcus uberis is Gram-positive, with a cell wall structure similar to Staphylococcus spp., as well as S. agalactiae and S. dysgalactiae. S. uberis is the most common Streptococcus species isolated from cases of mastitis. Petersson-Wolfe 2012, Streptococcus uberis fact sheet, Publication DASC-5P, Virginia Cooperative Extension. S. uberis is highly contagious and spreads from cow to cow during milking. Although associated with elevated somatic cell counts, streptococcal mastitis may not be detected by CMT because its limit of detection may be about 450,000 cells per ml. BTSCC is an accurate screen for herd-wide intramammary infection with Streptococcus uberis. Having a BTSCC above 250,000 is an indicator that a high number of cows have intramammary infections, for example, Streptococcus and Staphylococcus are the major causes of elevated cell counts. Streptococcus uberis may be treated using a broad spectrum antibiotic, for example, by intramammary infusion using a cephalosporin, such as ToDAY® cephapirin sodium, Boehringer Ingelheim Vetmedica, Inc., or SPECTRAMAST® DC ceftiofur hydrochloride, Zoetis. However, S. uberis may be resistant to certain antibiotic treatments. A Streptococcus uberis bacterin has been developed. Streptococcus uberis fact sheet, Hygieia Biological Laboratories.

Infection with S. agalactiae is associate with elevated somatic cell count and total bacteria count and a decrease in the quantity and quality of milk products produced. Keefe 1997, Can Vet J 38(7): 429-437. Streptococcus agalactiae is highly contagious and may cause a low grade persistent infection and does not have a high self-cure rate. When a herd is infected, traditionally there has been a high within-herd prevalence. Keefe 1997. Streptococcus agalactiae has the ability to adhere to the mammary tissue of cows and the specific microenvironment of the bovine udder is necessary for the growth of the bacteria. Methods for control include premilking teat disinfectant, postmilking teat dip and dry cow therapy (DCT). Streptococcus dysgalactiae therapy may include intra mammary infusion or systemic therapy of a broad-spectrum antibiotic. Petersson-Wolfe 2012, Streptococcus dysgalactiae fact sheet, Publication DASC-5P, Virginia Cooperative Extension. Antibiotic resistant strains have been noted. Keefe 1997.

Diagnosis

Mastitis may be diagnosed in various ways. First, the inflammatory response of the cow can be determined, through measuring the somatic cell count (SCC). Other parameters which may be used to diagnose clinical mastitis include, for example, N-acetyl-β-glucosaminidase (NAGase), milk amyloid A (MAA) level, serum amyloid A (SAA) level, and the level of proinflammatory cytokines interleukin or tumor necrosis factors, which may be identified, for example, by using a PCR assay. Kalmus et al., 2013, J. Dairy Sci., 96:3662-3670. Second, the detection of visible signs, such as swelling, redness, and hardness of the udder, represents an obvious, macroscopic way to assess udder health. A significant positive association has been identified between the severity of the clinical signs with inflammatory markers in the milk. Kalmus et al., 2013. A third parameter, possibly the most appreciable for the farmer, is milk production, indirectly related to udder health and several other disorders of infectious or metabolic origin. These 3 aspects are all expressions of an inflammatory or other physical reaction of the host. Vanderhaeghen et al., 2014; J Dairy Sci. 97:5275-5293.

Knowledge of the causative pathogens may be required for appropriate control and treatment of mastitis. Bacterial culture has been the gold standard for mastitis diagnostics (NMC, 2004), but a commercial PCR-based method has been introduced as a routine method for detection of mastitis-causing bacteria (PathoProof Mastitis PCR Assay; Thermo Fisher Scientific, Espoo, Finland). PathoProof Mastitis PCR assay is a real-time PCR for identifying 11 mastitis pathogens and the staphylococcal beta-lactamase gene. Due to the greater sensitivity of the PCR test compared with the conventional methods, often resulting in detection of more species per sample, the interpretation of the PCR results may be challenging (Koskinen et al., 2010).

Mastitis-causing bacteria entering the udder quarter via the teat canal, establish IMI with varying degrees of tissue injury. Tissue injury and inflammation initiate an acute-phase response (APR), which most commonly begins by releasing inflammatory mediators from tissue macrophages or blood monocytes that gather at the site of damage. An APR results in an increase in systemic and local concentrations of acute-phase proteins (APP). Two of those proteins, haptoglobin (Hp) and serum amyloid A, play a significant role in the early response of the mammary gland to pathogenic bacteria. Haptoglobin is diffused from blood into the milk, but also originates from milk. Local APR in the udder have mostly been studied using experimental models in which pathogenic bacteria such as Escherichia coli or staphylococci have been infused into the udder quarter. These studies showed that E. coli increases concentrations of APP in the milk to a greater extent than CNS or Staphylococcus aureus. A field study by Pyorala et al. (2011) concluded that the concentrations of Hp and MAA in milk vary depending on which pathogens are isolated. Concentrations of APP were the highest in cases where mastitis was caused by E. coli and significantly lower when mastitis was caused by streptococci or Staph. aureus. Milk amyloid A and Hp inflammatory responses were very mild in mastitis caused by CNS. N-Acetyl-β-d-glucosaminidase (NAGase) is an intracellular, lysosomal enzyme that is released into milk from neutrophils during phagocytosis and cell lysis, but also from damaged epithelial cells, indicating udder tissue destruction. Kitchen et al., 1984 J Dairy Res. 51:11-16. Milk NAGase activity correlates very closely with SCC and can be analyzed also from frozen milk samples (Kitchen et al., 1984).

The concentration of MAA in milk may be determined by any known method, for example, by using a commercial ELISA kit (Phase MAA Assay Kit; Tridelta Development Ltd., Maynooth, Co. Kildare, Ireland). Milk Hp concentrations (mg/L) may be determined by any known method, for example, the method of Kalmus et al. 2013, based on the ability of Hp to bind to hemoglobin and using tetramethylbenzidine as a substrate. The assay is meant to determine concentrations of Hp in the serum, but may be adapted to be used for milk. Optical densities of the formed complex were measured at 450 nm using a spectrophotometer. Lyophilized bovine acute-phase serum was used as a standard Kalmus et al., 2013.

Kalmus et al. 2013 reported that the quantity of bacterial DNA in milk samples was associated with concentrations of APP and NAGase activity in the milk. These indicators reflect the inflammatory reaction in the mammary gland, and their concentrations increased with increasing severity of mastitis. However, concentrations of APP and NAGase activity in milk significantly differed between different mastitis causing bacterial species. Indicators of inflammation in milk, such as APP concentration and NAGase activity, may be useful to complete and support the bacteriological diagnosis of mastitis. Kalmus et al. 2013, J Dairy Sci. 2013, 96: 3662-3670.

Somatic Cell Count

Somatic cell count in milk from individual cows generally is a useful tool for monitoring the probability of intramammary infection, but may be accompanied with bacteriologic culture of milk to determine whether contagious or environmental pathogens are responsible. Hoblet et al., 1988, Coagulase-positive staphylococcal mastitis in a herd with low somatic cell counts, J Am Vet Med Assoc 1988 Mar. 15; 192(6): 777-80.

Somatic cell counting (SCC) may be performed using an automated method. The majority of somatic cells are white blood cells (leukocytes) and a small number of cells from the udder secretory tissue (epithelial cells). They appear in large numbers to eliminate infections and repair tissue damage done by bacteria. Counting the cells thereby helps to indicate the presence of Mastitis in dairy cattle. Various automated instrumentation is available to determine SCC. For example, Fossomatic™ 7 or BacSomatic™ count somatic cells in raw milk. An individual cow SCC of 100,000 cells/ml or less may indicate an “uninfected” cow where there is no significant production losses due to subclinical mastitis. A threshold of 200,000 cells/ml may determine whether a cow is infected with mastitis. Cows with greater than 200,000 are highly likely to be infected in at least one quarter. Cows infected with significant pathogens have SCC of 300,000 cells/ml or greater. Milk with an SCC of 400,000 cells/ml or higher is deemed unfit for human consumption by the European Union.

The US milk quality monitoring system requires that approximately monthly samples, taken from farm bulk milk, be tested for bacteria and somatic cells. When a single bulk tank somatic cell count (BTSCC) exceeds 750,000/ml, it raises a concern. When two of the last four consecutive milk samples are above the limit, the producer is placed on notice and if three of the last 5 are above 750,000/ml the Grade A license is suspended until corrections are made and acceptable values (less than 750,000/ml) obtained. The US does not average several results from a particular time period; rather it uses the individual monthly cell count results. A trend to reduction in SCC may occur as a result of progressively severe payment schemes implemented by milk purchasing companies who penalize herds with a high BTSCC. Further, studies have shown that for every increase of 100,000 cells/ml above 150,000 cells/ml in BTSCC, there was a reduction of 1.5% in milk production. Milk Development Council, Desktop Review on Mastitis Management, Project 01/T6/03, 2010, AHDB Dairy, p. 7. Bulk Tank Somatic Cell Count (BTSCC) may also indicate presence of subclinical mastitis in a herd.

Direct microscopic somatic cell counting (DMSCC) may be employed, for example, using Rules for identifying and counting somatic cells single strip procedure (Form FDA-2400d). See Rules for Identifying Cell Count-FDA-DMSCC-2004.

The California Mastitis Test (CMT, also known as the California Milk Test) is a simple indicator of the Somatic Cell Count (SCC) of milk. It works by using a reagent which disrupts the cell membrane of somatic cells present in the milk sample; the DNA in those cells to reacting with the test reagent. It is a simple but very useful technique for detecting subclinical mastitis on-farm, providing an immediate result and can be used by any member of farm staff. It is not a replacement for individual laboratory cell count sampling, but has several important uses. A four-well plastic paddle is used, one well being used for each quarter of the cow to be tested. The foremilk is discarded, and then a little milk drawn into each well. An equal volume of test reagent is added and then the sample is gently agitated. CMT is a simple indicator of the somatic cell count in milk. It operates by disrupting the cell membrane of any cells present in the milk sample, allowing the DNA in those cells to react with the test reagent, forming a gel. Specifically a reaction of sodium hydroxide or an anionic surfactant and milk results in the thickening of mastitic milk. A dish detergent such as Fairy Dish detergent, Proctor & Gamble, may be employed as anionic surfactant. CMT provides a useful technique for detecting subclinical cases of mastitis. The reaction is scored on a scale of 0 (the mixture remaining unchanged) to 3 (an almost-solid gel forming), with a score of 2 or 3 being considered a positive result. This result is not a numerical result but is an indication as to whether the cell count is high or low; the CMT will only show changes in cell counts above 300,000. The advantage of the CMT over individual cow cell count results is that it assesses the level of infection of individual quarters rather than providing an overall udder result, enabling the problem quarter(s) to be identified. It also provides a ‘real-time’ result; laboratory testing provides a historical result as it can take days for lab results to be returned. A special reagent for the test is sold as ‘CMT-Test’, but domestic detergents (‘washing-up liquid’) can generally be substituted, being cheaper and more readily-available. https://dairy.ahdb.org.uk/technical-information/animal-health-welfare/mastitis/recordstools/test-kits/cmt-california-milk-esi. CMT test kits are available commercially, for example California Mastitis Test (CMT) Kit (Immucell).

The present disclosure relies upon a principle known as “bacterial replacement”, or “niche exclusion”, where one microorganism replaces and excludes another. In the field of ecology, competitive exclusion, or Gause's Law, states that two species that compete for the exact same resources cannot stably coexist. This is due to the fact that one of the competitors will possess some slight advantage over the other leading to extinction of the lesser competitor in the long run. In higher order organisms, this often leads to the adaptation of the lesser competitor to a slightly different ecologic niche.

Methods and compositions for durably managing the microbiome of a subject are provided. In embodiments, the microbiome is a dermal and/or mucosal microbiome (Exobiome). While methods to treat infection by a pathogenic microorganism exist, methods to prevent recurrence are effectively nonexistent.

One method comprises decolonizing heifers using a decolonizing agent, and recolonizing with a live biotherapeutic composition comprising a kill switched Staphylococcus aureus to prevent Staphylococcus infections from chronically infecting udders, causing intramammary infections, or skin and soft tissue infections. In another example, following milking and reserving a baseline milk sample for testing, a cow having a Staphylococcus aureus subclinical mastitis/intramammary infection may be cleaned in all four quarters to remove dirt and manure, followed by a broad spectrum antimicrobial, for example, a povidone-iodine teat dip for at least 15 to 30 seconds. The teats may be thoroughly cleaned, and the cow may be forestripped. The cow may then inoculated in all four quarters, for example, by intramammary infusion of a kill-switched therapeutic S. aureus microorganism. The inoculation cycle may optionally be repeated for from 1 to 6 milking cycles. The milk may be sampled and discarded for 1 or more weeks following first inoculation. The cow exhibits reduced somatic cell count after 1 week following first inoculation. The SCC may be reduced to no more than 300,000 cells/mL, 200,000 cells/mL, or preferably no more than 150,000 cells/mL.

Infectious Agent—Staphylococcus aureus (MSSA and MRSA)

Classified since the early twentieth century as among the deadliest of all disease-causing organisms, each year around 500,000 patients in hospitals of the United States contract a staphylococcal infection, chiefly by Staphylococcus aureus. Up to 50,000 deaths each year in the USA are linked with Staphylococcus aureus infections. Staphylococcus aureus exists on the skin or inside the nostrils of 40-44% of healthy people. Staphylococcus aureus is also sometimes found in the mouth, gastrointestinal, genitourinary, and upper respiratory tracts. Some studies indicate even higher colonization prevalence. For example, Eriksen et al maintain that there is a higher percentage of transient or intermittent carriers that increase the prevalence number; sometimes to greater than 75%.

Staphylococcus aureus 502a WT BioPlx-01WT® and Other Replacement and Blocking Strains

A Staphylococcus aureus 502a WT strain called BioPlx-01WT® is employed in example 1 and is a natural “wild-type” organism known to be relatively non-infectious, and which has no known side effects. It has been shown in BioPlx clinical studies to be highly effective in this intended application (occupying and blocking the required microbiomic niche to prevent the recurrence of MRSA).

The present methods prevent infection by durably replacing the (typically virulent and antibiotic-resistant) colonizing undesirable Staphylococcus aureus strain with a “blocking” organism—in this study the BioPlx01-WT Staphylococcus aureus 502a WT strain. This phenomenon is expected to be applied in a similar manner for any other pathogen replacement organism developed by BioPlx.

Other replacement strains such as synthetic strains are provided herein that are fully able to colonize the properly prepared skin and mucosal surfaces, and to occupy the ecologic niche used by this bacterial species, thereby blocking other variants from recolonizing that niche.

There are a very large number of Staphylococcus aureus variants (10,000+ genomes as of September 2017), as well as a wide range of genetic cassettes and virulence factors associated with this species.

Methicillin-resistant Staphylococcus aureus (MRSA) refers to a class of antibiotic resistant variants of this common human commensal and sometimes pathogenic bacteria. It varies from the wild-type strain (MSSA—Methicillin Sensitive Staphylococcus aureus) by its carriage of a mecA cassette that allows MRSA strains to produce an alternate penicillin binding protein (PBP2A) that renders them resistant to treatment with most beta lactam and many other first-line antibiotics.

Methicillin-Resistant Staphylococcus aureus (MRSA) and Virulent Methicillin-Susceptible Staphylococcus aureus (vMSSA) are virulent, invasive variants of Staphylococcus aureus that colonize many humans, and which can further cause both superficial soft tissue and severe systemic infections. Colonization with MRSA or vMSSA is usually a required precursor to active Staph infection. Infection is caused by the bacteria colony on the skin or mucosal membranes, penetrating the outer immunological barrier and invading tissue or the blood stream through a wound, an incision, a needle puncture, or other break in the skin. This can lead to bacteremia and other systemic infections that have high mortality rates.

The present disclosure uses a generally passive strain of Staphylococcus aureus to replace and exclude MRSA or vMSSA from its usual place in the dermal/mucosal microbiome. The wild type interfering Staphylococcus aureus used by BioPlx is known to be poor at causing systemic disease, however, regardless of the level of variance or invasiveness virtually any microorganism can become an “accidental pathogen” through natural or accidental inoculation. This is particularly true in the case of Staphylococcus aureus.

The decolonization and BioPlx01 strain application methods developed by BioPlx allows the strains provided herein a massive numerical and positional competitive advantage. The consequences of this method provide a much longer effect of MRSA decolonization than a simple antiseptic destruction of the virulent MRSA strain. Early studies show a greater than 6 month total exclusionary effect of the BioPlx01 MRSA decolonization/recolonization process with the BioPlx product as opposed to prior literature demonstrating 45% recurrence of Staphylococcus aureus nasal colonization at 4 weeks and 60% at 12 weeks with the standard decolonization method alone.

Overview of Indication

Staphylococcus aureus infections are a severe problem in both hospitals and community health settings. Methicillin-resistant Staphylococcus aureus (MRSA) is genetically different from other strains of Staphylococcus aureus, with genetic elements conferring resistance to the antibiotic methicillin and other (usually beta-lactam) antibiotics typically used to treat Staphylococcus aureus infections. MRSA strains carry a mecA expression cassette that allows MRSA strains to produce an alternate penicillin binding protein (PBP2A), and it's this mutation that confers resistance. Due to this resistance, MRSA is difficult to treat, making it a life-threatening problem in many cases. MRSA is frequently contracted in hospitals or other types of healthcare settings (Hospital Associated [HA]). These infections typically occur at the time of an invasive procedure such as surgery, intravenous catheterization, intubation, or artificial joint placement. Community-associated (CA) MRSA is typically spread by skin-to-skin contact, and the first symptoms tend to be large boils on the skin.

The BioPlx method using BioPlx strains is not a treatment for invasive MRSA disease, and therefore is not intentionally applied to a patient during the invasive disease state. The benefits of the BioPlx method can be demonstrated in a patient group that: 1) is at high risk for invasive disease, 2) has high morbidity and mortality from this increased risk to show significant clinical benefit, and has no other effective options for the prevention of invasive Staphylococcus aureus disease. These characteristics define the group of patients that the Centers for Disease Control have been tracking regarding the MRSA subset since 2005 who have already experienced invasive MRSA disease—72,444 according to ABC surveillance data in 2014.

The surface of the human skin and mucosal layer where Staphylococcus aureus resides in the colonization state has a very different level of required nutrients as well as different environmental qualities than that inside the human body. It has been widely recognized that in order for bacteria to be successfully invasive, they must be able to adjust their needs and responses between the colonization and invasive states. This is accomplished by the bacterium sensing the changes between these environments and switching on or off certain gene cassettes allowing for the production of proteins more adapted to the new invasive state.

The BioPlx method, and specifically BioPlx01 strains, take advantage of this requirement by rearranging molecular instructions leading to the death of the organism in the operons of one or more of these specific cassettes. This creates a “holding strain” of colonizing Staphylococcus aureus that is unable to cause disease in the patient to whom it is introduced, but also does not allow other circulating Staphylococcus aureus strains that may normally colonize the human population to colonize this patient. This occurs through the ecologic premise of competitive exclusion.

The current “Standard of Care” for patients colonized with MRSA is not uniform. There are no guidelines as to the management of staphylococcal colonization in patients that are at high risk of recurrent disease. The IDSA Clinical Practice Guidelines for the Treatment of MRSA Infections in Adults and Children in 2011 provide only C-III level (the lowest—no data, expert opinion) support for decolonization procedures in patients with recurrent community-acquired skin and soft tissue infections and make no mention of the role of decolonization in the prevention of invasive MRSA disease. Some hospitals have pursued a broad screening and isolation program for all admitted patients to their institution, but this has not been shown to be effective owing to (including) poor durability of effect and lower baseline risk of the average hospitalized patient (i.e. UC Irvine MRSA outbreak.) Other hospitals therefore have reduced their attention to patients admitted to the ICU and cardiothoracic surgery cases only. This strategy has been shown to reduce MRSA clinical isolates as well as bloodstream infection from any pathogen. However, these are short term situational strategies designed to reduce risk of MRSA infection over a near time frame.

MRSA disease and colonization is a complicated epidemiologic problem for both the United States and the rest of the world. The manifestations of MRSA are broad from asymptomatic colonization to invasive disease states conferring high mortality and cost to the system. It is clear that the MRSA patients that have experienced invasive disease is medically distinct. They have a higher mortality than any other MRSA subpopulation. They have a higher treatment failure rate. They have a much higher risk for another invasive MRSA incident than any other group of patients. This makes this group an appropriate orphan group toward which the BioPlx method should be directed, and which would benefit from its use.

It can be concluded that decolonization is largely ineffective in durably clearing MRSA colonization, and leads to a high rate of recurrence. We have found that only decolonization in conjunction with active recolonization provides long term conversion from one organism (variant) to another.

Recurrent Invasive MRSA as a Clinically Distinct Disease

Another indication is “prevention of recurrent invasive MRSA.” Patients who have already experienced an episode of invasive MRSA infection have a greatly increased susceptibility to a subsequent invasive MRSA infection. The BioPlx technology provided herein works by occupying the niche in the microbiome that would normally have the potential to be occupied by a virulent form of MRSA.

Invasive MRSA-Caused Systemic Infection:

SA, including the variant MRSA, can exist in harmless coexistence on the surface of the skin and mucous membranes of at least 40% of all humanity, so the bacterium itself is not descriptive of disease; rather, its clinical presentation is definitional.

The whole of national and international authorities that define and monitor this condition concur that invasive MRSA infection is a separate and distinct disease from other conditions caused by this bacterium.

Simple colonization with any type of Staphylococcus aureus should not be considered a disease state. In fact, those humans with nutritional and environmental characteristics of their skin and mucosal biomes that are hospitable to Staphylococcus aureus must have some such niche occupant as part of their microbial flora to achieve a stable balanced “resting state” of their biome. The goal of any method would be to durably replace a MRSA strain on an at-risk patient with the product strain—in this case an antibiotic sensitive Staphylococcus aureus modified to be unable to survive within the human body in the invasive state.

To create invasive infectious disease, MRSA must abandon its passive commensal status, and breach the dermal/mucosal barrier, entering into the subdermal interstitial (interstitial fluid) or circulatory (blood, serum, plasma) areas. This “state change” initiates a new disease state, with new organism behaviors and relationships to the host.

Staphylococcus aureus bacteremia (SAB) is an important instance of this type of infection with an incidence rate ranging from 20 to 50 cases/100,000 population per year (ranging from 64,600 to 161,500 cases per year). Between 10% and 30% of these patients will die from SAB. Invasive systemic MRSA bacteremia has a mortality rate of around 20%. Comparatively, this accounts for a greater number of deaths than for AIDS, tuberculosis, and viral hepatitis combined.

The latest report for which there is a CDC-US national case estimate for invasive MRSA disease (2014) is 72,444 cases. The number of patients with this disease is less than 200,000 per annum, and it may permit an orphan drug designation. MRSA can impact patients at three distinct levels: 1) colonization, 2) superficial infection—skin and soft tissue, and 3) systemic invasive infection.

1) Colonization. Staphylococcus aureus is a normal commensal organism permanently colonizing around one third of the human population, with transient colonization occurring in about one additional third of the population. MRSA variants of this organism occupy organism the microbiome niche, and have colonized approximately 2% of the population in the US (with a high degree of variability depending on location and occupation). MRSA colonization creates a standing reservoir of potentially infectious organisms located directly on the outer layer of our immune/defense system, and this poses an ongoing risk to the patient.

2) Superficial infection—skin and soft tissue infection. Skin-associated MRSA or skin and soft tissue infection is the most common of the two major disease state categories. It typically starts as a swollen, pus or fluid filled, boil that can be painful and warm to the touch, and at times accompanied by a fever. If left untreated, these boils can turn into abscesses that require surgical intervention for draining. For MRSA that's confined to the skin, surgical draining of abscesses may be the only necessary treatment, and antibiotics are not indicated. Skin and soft tissue infections are treated by surgically draining the boil and only administering antibiotics when deemed absolutely necessary.

3) Systemic invasive infection. MRSA bacteremia (invasive MRSA) is a systemic MRSA infection that is defined as the presence of MRSA in typically sterile sites, including the bloodstream, cerebrospinal fluid, joint fluid, bone, lower respiratory tract, and other body fluids. MRSA bacteremia has a far worse prognosis compared to MRSA infections confined to the skin, with 20% of cases resulting in death. The difference in prognosis, location of the infection, and clinical symptoms of the condition make it clinically distinct from skin and soft tissue infection MRSA infections. MRSA bacteremia causes multiple complications not seen in skin and soft tissue infections, including infective endocarditis, septic arthritis, and osteomyelitis. For invasive MRSA, daptomycin and vancomycin are recommended treatments in the U.S. Vancomycin has a relatively slow onset and poorly penetrates some tissues. Daptomycin has been shown to be effective, but treatment-emergent nonsusceptibility is an issue, in addition to the issue of vancomycin encouraging daptomycin resistance in MRSA. The difference in clinical symptoms as well as treatment methods for invasive MRSA provides clear evidence for invasive MRSA as a clinically distinct condition from MRSA Skin and soft tissue infections.

The BioPlx technology works by preventing the recurrence of an invasive MRSA infection in those who have been colonized (including those that have already experienced an invasive MRSA infection) and who have undergone a decolonization procedure. As a decolonization/recolonization microbioic method, the BioPlx technology would not be administered to “treat” a patient while they had a systemic MRSA infection. It would be applied subsequent to the clearance of a systemic MRSA infection (and a full body decolonization).

It is an established principle of medical nomenclature that a disease or condition is not simply synonymous with the causative agent. In the present case, MRSA-mediated systemic bacteremia (or other designations of invasive systemic disease) is unambiguously distinct from the other superficial skin and mucosal conditions that may be caused by, or associated with, MRSA, or by other Staphylococcus aureus strains. Invasive systemic MRSA-mediated disease has a clearly distinct diagnosis, pathology, treatment, and prognosis profile.

It's important to note that, based on the mechanism of action of BioPlx01 strains, patients are prevented from subsequent systemic MRSA infection, as opposed to treatment of invasive MRSA infection per se. So, “prevention of recurrent systemic MRSA infection” would be the most accurate description of the indication for BioPlx01 strains.

The target population of patients that have had invasive MRSA Infection, have been successfully cleared of the organism (typically through standard antibiotic intervention (e.g. Vancomycin), and yet have a high risk (rate) of MRSA recolonization, recurrence and the associated elevated risk of MRSA systemic reinfection.

International and US Recognition of the Disease Designation:

A clear definition of this disease is put forth by the Centers for Disease Control and Prevention (CDC) as it has been actively monitoring this condition in the United States since 2005. The agency performs this monitoring utilizing the Active Bacterial Core surveillance system via the Emerging Infections Program (EIP). A case in this context is defined by the isolation of MRSA from a normally sterile body site. Normally sterile sites included blood, cerebrospinal fluid, pleural fluid, pericardial fluid, peritoneal fluid, joint/synovial fluid, bone, internal body site (lymph node, brain, heart, liver, spleen, vitreous fluid, kidney, pancreas, or ovary), or other normally sterile sites.

The CDC also created the National Healthcare Safety Network (NHSN) as a tracking system for more than 16,000 US healthcare facilities to provide data to guide prevention efforts. The Center for Medicare Services (CMS) and other payers use this data to determine financial incentives to healthcare facilities for performance. The system tracks MRSA bloodstream infections as a marker for invasive disease for epidemiologic purposes.

The MRSA mediated invasive disease state is also codified in the ICD9 and ICD10 system by a grouping of conditions each with their own numeric code specific for the causative agent MRSA. For example, sepsis due to MRSA is coded A41.02, pneumonia due to MRSA is coded J15.212. This further exemplifies the differential characterization that invasive MRSA disease is given in juxtaposition to superficial skin and soft tissue disease due to the same agent—code L03.114 (left upper limb example) with the follow code of B95.6 MRSA as the cause of disease classified elsewhere, which is attached to a variety of other infection codes to indicate MRSA as the cause of the disease condition.

The European Center for Disease Control (ECDC), a branch of the EU also surveilles invasive Staphylococcus aureus isolates by similar definition to the NHSN and tracks methicillin-resistance percentages but the reporting requirements do not produce an EU estimate of total annual cases.

Differentially, unlike systemic conditions, simple MRSA colonization is not itself typically regarded as a disease. Colonization however is considered a precondition for most invasive disease, as evidenced (for example) by studies that show that nasal Staphylococcus aureus isolates are usually identical to strains later causing clinical infection. This persistent colonization state reflects the ecological stability of this bacteria on skin and mucosal surfaces.

This colonization state is recorded in the ICD10 system, Z22.322, under the Z subheading which is reserved for factors influencing health status and contact with health services but not an illness or injury itself.

The Target Orphan Disease Population:

The orphan disease population targeted for the BioPlx non-recurrence method is the group of people previously invasively infected (systemic infection) with MRSA (a population known to be susceptible), and who continue to suffer ongoing recolonization with MRSA. CDC monitors all U.S. cases of invasive MRSA infection. Multiple researchers have described this medically distinct population—patients who have already suffered one defined episode of invasive MRSA infection. This group is at increased risk for life threatening invasive disease as a result of their demonstrated susceptibility and their continued colonization.

In some embodiments, a method is provided for preventing recolonization, or preventing recurrence of MRSA-caused systemic invasive bacteremia, comprising prevention of (or prevention of recurrence of) a prerequisite MRSA colonization by

1) decolonization of MRSA from mucosal and dermal microbiomes, and
2) recolonization of these microbiomes with a synthetic Staphylococcus aureus (e.g., a BioPlx01 strain). The method is effective, through the effect of bacterial interference, operating through niche dynamics within the target dermal/mucosal microbiome ecosystem, because the synthetic Staphylococcus aureus (e.g., a BioPlx01 strain) serves to occupy specific niches, and thus blocks/prevents MRSA recolonization (blocks recurrence). The efficacy of this method has been demonstrated clearly in proof of principle studies provided herein.

SA is present as part of the normal microbiome of more than 40% of the total human population. The MSSA colonization state is common. The MRSA variant is found on around 1-2% of the US population, but in certain areas or demographics this level can be considerably higher. It is thought that MRSA has the ability colonize anyone within the Staphylococcus aureus susceptible population. Staphylococcus aureus lives most commonly on the surface of the skin and in the anterior nasal vestibules, but can also be found in smaller amounts in the deep oropharynx and gastrointestinal tract and in normal vaginal flora in some individuals.

In colonized individuals Staphylococcus aureus usually remains a non-invasive commensal bacterium simply occupying an ecologic niche and not causing disease. In a portion of those colonized however, this bacteria can cause disease either opportunistically or as a result of the increased likelihood of invasion due to some particular variant characteristics.

Approximately 23% of persistent MRSA carriers developed a discrete MRSA infection within one year after identification as a carrier.

Many Staphylococcus aureus variants have acquired genetic cassettes coding for virulence protein products that allow such strains to more effectively invade through the epidermal or mucosal tissue layers, and subsequently initiating deep or systemic infection. In colonization or infection the presence of the mecA cassette limits the treatment options for these patients, and a number of studies have documented the increased mortality rate associated with MRSA when compared to MSSA in bacteremia, endovascular infection and pneumonia.

It is not possible to predetermine whether an individual who is colonized with MRSA will eventually progress to invasive disease or not, so it is particularly important to identify and treat the entire population of patients who have a well-documented increased risk for invasive MRSA disease.

MRSA-Mediated Invasive Disease Statistics:

MRSA was identified by British scientists in 1961 and the first American clinical case was documented in 1968. For the next 25 years, MRSA was regarded largely as an endemic hospital-based problem that was increasing in incidence, however starting in the mid to late 1990s, an increase of incidence of community-associated MRSA was seen mostly manifesting in superficial skin and soft tissue infections. Of greatest concern to the medical community has been the increase in invasive infections caused by MRSA. The increasing trend in incidence of invasive MRSA disease was seen throughout the 1990s and peaked in 2005.

The CDC tracks the incidence of invasive MRSA disease through the NHSN and the Emerging Infections Program—Active Bacterial Core surveillance system also starting in 2005. As compared to 2005, 2015 data shows that the overall incidence for invasive MRSA disease has decreased almost 50% from an incidence rate of 37.56 to 18.8. Expensive and laborious infection control interventions enacted in hospitals in response to this public health crisis has been given much of the credit for the decreased incidence, as the majority of the gain was seen in health care associated cases as opposed to community associated ones. Despite the gains that have been made over the past decade, invasive MRSA infections continue to be a prioritized public health issue. These infections can be very difficult to treat and treatment failure has been shown in nearly 25% of patients on proper therapy. Predicting which health care experienced patients are at risk for invasive MRSA is a challenging problem. Risk factors such as MRSA colonization, the presence of chronic open wounds and the presence of invasive devices have been elucidated.

The presence of these characteristics alone do not predict which patient will ultimately display invasive disease. However, one of the most predictive risk factors for a patient getting an invasive MRSA infection is having had a previous invasive MRSA infection. In the 2004-2005 data from the Active Bacterial Core Surveillance (ABCs) it was noted that almost 13% of their invasive cases went on to develop a second invasive MRSA infection during the 18 months of retrospective data evaluation. Another look at the EIP-ABC data in the calendar year 2011 found that 8% of these patients had more than one invasive MRSA infection separated by at least 30 days. The longer term risk of recurrent invasive MRSA infection is surely greater still as these estimates will miss earlier infections in these patients prior to the study time period and later ones that occur after the end date. Since Huang and Platt (2003) showed that 29% of hospitalized patients with known MRSA colonization or infection went on to develop a second MRSA infection (often severe) within an 18 month follow up, targeting this group to prevent recurrence of the invasive disease state could prevent approximately 17,500 subsequent invasive MRSA infections (using the most recent CDC data).

Invasive MRSA and skin and soft tissue infection from MRSA are both caused by the same pathogen. However, orphan designations are awarded based on the dyad of drug and disease. MRSA is a pathogen, and not a disease state. However, it can cause infection, and it's these different types of infectious disease that are being treated. Invasive MRSA comes with a far more severe prognosis as well as different clinical manifestations from MRSA confined to the skin or simply being colonized with MRSA. About 40% of the U.S. population is colonized with Staphylococcus aureus, typically found in the nose or on the skin. Generally, there are no signs of infection that would be considered “a disease state.” However, systemic MRSA infection will manifest as high grade fever, chills, dizziness, chest pain, swelling of the affected area, headache, rash, cough, and other systemic symptoms. These two conditions are treated differently, where skin and soft tissue infections are typically treated by incising and draining the boils commonly associated with skin and soft tissue infections. Antibiotics and decolonization are only employed if there are signs of systemic or severe disease that has spread to multiple sites.

Invasive MRSA has an incidence rate of 20 to 50 cases/100,000 people per year.^{6a} With a current U.S. population of 326,199,002 (accessed on Nov. 2, 2017 from www.census.gov/popclock), this means there are 163,100 cases of invasive MRSA infection in the U.S. per year conservatively, falling below the 200,000 patient criteria for FDA orphan designation. We searched for other sources of reported prevalence to confirm that we had calculated the most conservative estimate of this patient population. Hassoun et. al reported an incidence of 72,444 cases of invasive MRSA in the U.S. in 2014, which had decreased from 111,261 in 2005.^{7a} Based on this, and assuming that the population will continue to decrease, we can assume that a prevalence of 163,029 patients with invasive MRSA in the U.S. in 2017 is a very conservative estimate. According to the CDC, there were more than 80,000 invasive MRSA infections and 11,285 related deaths in 2011.

To address this problem the present inventors have developed BioPlx01 strains, molecularly-altered strains of Staphylococcus aureus that are unable to cause disease but can reside in the microbiome niche that MRSA could take hold in. The lack of invasiveness of BioPlx01 strains is made possible by operons that are turned on upon contact with blood or plasma, triggering the death of the organism. A patient who has tested positive for MRSA and is experiencing systemic symptoms will undergo a full body decolonization before the BioPlx01 strain is administered, allowing it to occupy the niche that MRSA would have previously occupied in that patient's microbiome. By preventing virulent strains of MRSA from occupying the niche, these virulent strains cannot colonize, and subsequently invade sterile tissue sites. BioPlx01 strain is able to prevent recurrent systemic MRSA infections.

In one embodiment, a method for treatment of Staphylococcus aureus lung infections in patients with cystic fibrosis is provided.

In one embodiment, a method for treatment of Invasive Bacteremia is provided. Using the criteria adopted by CDC (Centers for Disease Control and Prevention), Invasive Bacteremia is indicated by the isolation of bacteria from a normally sterile body site. These may include blood, CSF, joint fluid, bone samples, lower respiratory tract samples and other sterile body fluids. This condition is related to, but is clearly distinguished from, simple bacterial colonization and bacteria mediated skin and soft tissue infection. It is accepted that the colonization state is a prerequisite for invasive disease in the vast majority of cases.

MRSA and v-MSSA Mediated Invasive (Systemic) Bacterial Infection

Mediated by Staphylococcus aureus, MRSA Invasive Bacterial Infection may also be referred to commonly or in the literature as: MRSA bacteremia or sepsis, Systemic MRSA infection, MRSA bloodstream infections, invasive MRSA infection. Specific MRSA induced systemic conditions range from osteomyelitis, septic arthritis, pneumonia, endocarditis, bacteremia, toxic shock syndrome, to septic shock. The development of a method to prevent or reduce the recurrence of invasive MRSA disease in high-risk populations, through the mechanism of durably interfering with colonization of undesirable strains, would be a significant advance in the prevention of conditions typically required for invasive MRSA infection, and would reduce the likelihood of these patients suffering a subsequent invasive MRSA infection.

One objective of the present disclosure is to evaluate the BioPlx-01 WT material's ability to prevent the recurrence of MRSA in active healthy adult medical workers. This population is particularly at-risk for MRSA infection and has amongst the highest rates of MRSA colonization of any demographic. Successfully demonstrating a protective effect for this group would validate BioPlx-01 WT's efficacy in being able to prevent MRSA recurrence amongst effectively all those who are at risk.

“Recurrence” simply means “the bug comes back”. Recurrence is of central importance to both disease evolution and control. With recurrence, the pathogen comes back again and again, and each time it goes through a survival cycle it “learns” to be more and more resistant to the antibiotics it has seen. Without this recurrence, once the pathogen is gone, it would stay gone, and that would be that. If there were no recurrence, there would be no pressure to evolve toward antibiotic resistance.

In various embodiments, the subject may be colonized with one or more pathogenic microorganisms. In certain embodiments, the undesirable microorganism is a drug-resistant pathogenic microorganism. The drug-resistant pathogenic microorganism may be selected from a Neisseria gonorrhoeae, fluconazole-resistant Candida, MRSA, drug-resistant Streptococcus pneumoniae, drug-resistant Tuberculosis, vancomycin-resistant Staphylococcus aureus, erythromycin-resistant Group A Streptococcus, and clindamycin-resistant Group B Streptococcus. https://www.cdc.gov/drugresistance/biggest_threats.html.

In one embodiment, the undesirable microorganism may be a drug-resistant pathogenic Staphylococcus aureus.

Staphylococci are the most abundant skin-colonizing bacterial genus and the most important causes of nosocomial infections and community-associated skin infections. The species Staphylococcus aureus may cause fulminant infection, while infections by other staphylococcal species are mostly subacute. Colonization is usually a prerequisite for infection. Otto 2010, Expert Rev Dermatol 2010 April; 5(2):183-195. However, not all invasive Staphylococcus aureus infections are preceded by detected colonization with identical strain. The non-correlative fraction may be explained either by the “direct inoculation” or “direct wound seeding” theory such as an intraoperative event from a second carrier, or incomplete detection of all of these patient's Staphylococcus aureus strains in colonization or colonization with the invasive strain in the time since the initial colonization surveillance.

SA is a common human commensal organism that is present (colonizes), typically without symptoms, in 30 to 50% of the (US) population. The asymptomatic carriage of Staphylococcus aureus by humans is the primary natural reservoir, although domestic animals, livestock, and fomites may serve as adjunctive reservoirs.

There are many different strains of Staphylococcus aureus, many of which can also act as serious pathogens. Symptoms of Staphylococcus aureus infections can be diverse, ranging from none, to minor Skin and soft tissue infections, to invasive life-threatening systemic disease such as endovascular infections, pneumonia, septic arthritis, endocarditis, osteomyelitis, foreign-body infections, sepsis, toxic shock and endocarditis. The anterior nasal mucosa has traditionally been thought to be the most frequent site for the detection of colonization of healthy carriers with Staphylococcus aureus. Several sites may become asymptomatically colonized including the nares, throat, axilla, perineum, inguinal region, and rectum.

MRSA isolates were once confined largely to hospitals, other health care environments, and patients frequenting these facilities. Since the mid-1990s, however, there has been an explosion in the number of MRSA infections reported in populations lacking risk factors for exposure to the health care system. This increase in the incidence of MRSA infection has been associated with the recognition of new MRSA clones known as community-associated MRSA (CA-MRSA). CA-MRSA strains differ from the older, health care-associated MRSA strains; they infect a different group of patients, they cause different clinical syndromes, they differ in antimicrobial susceptibility patterns, they spread rapidly among healthy people in the community, and they frequently cause infections in health care environments as well. David, Michael et al., 2010, Clin Microbiol Rev 23(3): 616-687.

Why recurrent CA-MRSA Skin and soft tissue infections are common is not known. The mechanism by which recurrence occurs is unclear. Possibilities include reinfection from persistent asymptomatic CA-MRSA carriage or after acquisition from environmental MRSA or after new MRSA acquisition from close human or animal contact. Skin and soft tissue infections caused by MSSA also recur but less frequently than those caused by MRSA.

Under constant antibiotic pressure, many Staphylococcus aureus variants have developed antibiotic resistance. Today penicillin resistance in Staphylococcus aureus is virtually universal, and general beta-lactam and related multi-antibiotic (methicillin) resistance is now widespread, creating a significant new class of antibiotic-resistant “super-bugs”.

The pathogenic Staphylococcus aureus may be a drug-resistant Staphylococcus aureus, such as MRSA, or a vancomycin-resistant strain, such as VISA or VRSA. Alternatively, the pathogenic Staphylococcus aureus may be a virulent methicillin-susceptible Staphylococcus aureus (v-MSSA). v-MSSA is a high-virulence cause of life-threatening invasive infections. MRSA and v-MSSA are epidemic, and have a high human cost.

MRSA has become a serious public health problem in hospitals, clinics, prisons, barracks, and even in gyms and health clubs around the world. MRSA is a common cause of hospital-acquired infections (500 k US patients/year), and increasingly, of community acquired infections which can be serious. For systemically invasive disease—20% of cases result in death. MRSA is one of the most significant of the new antibiotic-resistant “super-bugs”. While methods to treat Staphylococcus aureus infection exist, methods to prevent recurrence are effectively nonexistent. Recurrence of MRSA skin infections is found in 31% to 45% of subjects.

One effort to prevent recurrence includes decolonization. The first (and currently only) widely practiced step for preventing recurrence is decolonization. Unfortunately, simple decolonization is poor at preventing recurrence. Doctors can initially treat the microbial colonization or infection—for example MRSA or v-MSSA colonization/infection—with topical chemicals (e.g. chlorhexidine) or antibiotics. In many cases treatment with antibiotics may “clinically” eliminate the disease. Antiseptics and astringents may be used for decolonization (i.e., suppression) including tea tree oil and chlorhexidine. Antibiotics used for suppression include topical antibiotics for nasal decolonization such as mupirocin. Systemic antibiotics most frequently used for MRSA include vancomycin, first generation antibiotics such as cefazolin, cephalothin, or cephalexin; and new generation antibiotics such as linezolid or daptomycin. In less serious MRSA cases, clindamycin or lincomycin may be employed. Nonetheless, with this decolonization alone the MRSA and v-MSSA pathogens typically recur- or grow back—nearly ½ of the time. This level of performance has naturally led to skepticism as to the efficacy of simple decolonization in preventing recurrence.

Clinicians often prescribe topical, intranasal, or systemic antimicrobial agents to patients with recurrent skin infections caused by methicillin-resistant Staphylococcus aureus (MRSA) in an effort to eradicate the staphylococcal carrier state. Some agents can temporarily interrupt staphylococcal carriage, but none has been proved effective for prevention of skin infections caused by MRSA. Creech et al. Infect Dis Clin North Am. 2015 September; 29(3): 429-464.

In both the literature and in the hands of the present inventors, it has been found that the quality of decolonization is correlated to the recurrence rate observed, but simple decolonization rarely resulted in a durable, successful, outcome.

The present disclosure provides methods and compositions focused on preventing recurrence through the effective and durable modification of microbiome populations.

Methods for preventing or decreasing recurrence of a pathogenic microbial infection have been developed comprising suppressing a microbial infection or colonization.

A method to decrease recurrence of a pathogenic infection or decrease colonization of a undesirable microorganism in a subject is provided, comprising decolonizing the undesirable microorganism on at least one site in the subject to significantly reduce or eliminate the presence of the undesirable microorganism from the site; and replacing the undesirable microorganism by administering to the subject a synthetic second microorganism having the same genus and species as the undesirable microorganism.

The methods and compositions to prevent recurrence include replacement of the pathogenic microorganism by filling the biome niche occupied by the pathogen with a specially designed synthetic microorganism—or “good bug”. By occupying the same biome niche, the “good bug” crowds out the pathogen, preventing it from recolonizing, or moving into (or back into) its preferred ecological neighborhood. One way to ensure the same biome niche is filled is by designing a synthetic microorganism starting from the same genus and species as the pathogenic microorganism.

The methods and compositions to prevent recurrence include promoting or supporting the synthetic microorganism—the “good bug”—by re-establishing key nutritional, chemical, or commensal environments that further promote the preferred organism and inhibit recolonization by the pathogen. For example, a commensal cluster may provide further layered defense in preventing the pathogen from moving back into its old ecological niche—it may help prevent recurrence.

The BioPlx method is enabled by state of the art methods/technologies including microbiomics, systems & computational biology; environment interactions (clusters & signaling); proprietary organisms (selected & modified); and variant and strain substitution strategies.

Replacement microorganisms are provided herein including (1) “BioPlx01-WT® variant”—a Staphylococcus aureus 502a wild-type microorganism with an established history of non-virulence and passive colonization which has been isolated, verified, and prepared for field trials using this strain cluster as described in Example 1; (2) “BioPlx01-KO® engineered variant”, a synthetic Staphylococcus aureus strain that enhances safety by knocking out specific virulence genes; and (3) “BioPlx01-KS® engineered variant”, a synthetic Staphylococcus aureus strain that embeds a molecular programmed cell death trigger to prevent invasive virulence. In some embodiments, the synthetic microorganism acts purely as a substitution for the pathogenic strain, without “new” infection or colonization.

An extensive proprietary library of fully characterized Staphylococcus aureus cultures (strains and variants) has been developed which is used for replacement organism sourcing; used for durability and competition analysis; used for Genotype/Phenotype comparative analysis; used for virulence genome/transcriptome clustering modeling; and used for signaling genome/transcriptome clustering modeling.

A Library of controlled commensal organisms is being developed for potential variant cluster co-administration with the BioPlx01-KS® variant.

Methods for Computational Microbiology are also being developed including Machine Learning; Modeling of complex dynamic microbiomic systems; Genome/Transcriptome/Proteome (Phenotypic) relationships; Virulence factor genetics and promoters; Modeling resilience and changes over time/condition; n-dimensional niche-forming relationships; and High dimensional cluster relationships.

Central to the present model anti-recurrence method is the principle of “non-co-colonization”, meaning that only one species, and one variant of that species, can occupy the relevant skin or mucosal biome ecological niche at any one time. Underlying this simple and testable phenomenon are a host of deeper generative principles that combine to shape the emerging science of Microbiomics. Although widely generalizable, discussion of non-co-colonization in this section refers specifically to Staphylococcus aureus colonizations.

Non-Co-Colonization

The principle of non-co-colonization (also known as “bacterial replacement”) states that only one variant/strain of one species can occupy any given niche within the biome at any given time.

The central empirical phenomenon of non-co-colonization represents an aggregate effect: the consequence of the interaction of a large number of forces that can be found operating in complex systems, and which are only today becoming well characterized and mathematized.

Bacterial niches within the human biome that are specific to the species level underlie the present technology. If there were no specificity to biologic niche occupation, then intentional strain exchange would not be achievable, as would the experimentally demonstrated phenomenon of bacterial replacement.

Expectations for non-co-colonization are important for durability of the present method for prevention of recurrence of pathogenic colonization or infection. Variant-to-variant non-co-colonization has been demonstrated experimentally in the literature with strain/variant substitution (e.g., the Staphylococcus aureus 80/81 to 502a conversions of Shinefield et al., 1963) and has been confirmed in present clinical studies, as shown in Example 1.

Sustained species-to-species niche occupation is suspect because careful reading of the literature indicates that durability is low, and in vivo evidence is rare. A transient occupation may occur, but is not considered to be an important outcome, as we are only interested in durable outcomes.

Failure of durability in species-to-species substitution serves as evidence that specific niche-filling requires a “close variant” substitution. This is significant as only durable biomes can display the functional characteristics (such as resilience) required for an effective non-recurrence technology/product.

In the case of variant-to-variant replacement, such as that seen in the present disclosure with respect to MRSA anti-recurrence materials, no direct evidence from the literature has been identified as to whether the replacement requires a “biome disruptive event” (such as accidental or intentional decolonization by antimicrobials, antibiotics, etc.) or whether it can occur via a “slow competitive replacement” (one organism out competing another for resources, growth, etc.). However, overwhelmingly in human dermal biomes, only one strain colonizes a person “in toto”, indicating that slow competitive replacement occurs. Further, the 55% success rate of anti-MRSA decolonization methods show that “biome disruptive events” can also induce durable biome changes. Both of these phenomena are expressions of non-co-colonization.

Non-co-colonization occurs in nature, for example, in the vast majority of cases only one variant of Staphylococcus aureus is detected within a single biome (over 95% of cases, with the balance likely caused by “transient conditions”).

In specifying and evaluating non-co-colonization durability (efficacy) it is necessary to recognize three distinct scales of outcomes: (1) short-term—immediately post recolonization, (2) early stable stage—after shedding excess organisms, and (3) long-term—after a stable “new” biome is established.

In the short-term—immediately post recolonization, the decolonized biome is dominated by organisms applied “in excess” during recolonization—generating a type of adventitious and transient binding (like spreading peanut butter). Testing within this period can only confirm that the biome application has occurred. Duration=a few days, with subsequent shedding of excess organisms.

In the early stable stage—after shedding excess organisms, the biome per se is reestablishing its equilibrium state, but ostensibly with the replacement organism rather than the pre-existing pathogen. Confidence in this outcome is primarily due to the overwhelmingly large ratio (probably millions to one) of new organisms to surviving post-decolonization pathogens. It is expected that this will become a stable colonization with a high level of durability. Testing at this period would confirm that MRSA or vMSSA has been eliminated, and replacement strain has been re-colonized. Duration=weeks to months.

In the long-term—after stable “new” biome established will demonstrate not only the organism's ability to occupy or “take” a niche, but its ability to “hold” that niche. In some embodiments, this stage is used to evaluate how competitive the replacement strain or synthetic microorganism is against the current generation of new biome invaders (such as USA300). This question refers to the “new” replacement organism's ability to compete over time against a slow competitive replacement as well as by external forces that could be biome disruptive over time such as antibiotic or antiseptic exposures or frequent re-exposure to the pathogen—especially if the strains are differentially sensitive to this disruptor.

It is important to characterize the phenomenon of microorganism variant non-co-colonization, variant-versus-variant niche occupation, and the empirical evidence already developed that this phenomenon exists and is a strong force in the dermal biome ecosystem.

The law of “competitive exclusion” refers to the situation where only one organism dominates one niche.

One historical error in understanding this phenomenon is assuming this is a binary system, conceptually driven by either one or two variants. In fact, a large number of different microorganisms, for example various Staphylococcus aureus strains may be environmentally present at any one time, and over time.

It may be concluded that without the phenomenon of non-co-colonization, virtually all “staph-capable” biomes would inherently be highly variable mixed heterologous “soups” of multiple variants. Various possibilities are shown in Table 1.

TABLE 1
Staphylococcus aureus (SA) niche
compatibilities and expected outcomes

| | Niche | Competitive | |
|---|---|---|---|
| case | compatibility | exclusion | Expected Outcome |
| 1) | one | + | one variant dominates |
| | Staphylococcus | | (except transitional) |
| | aureus niche | | |
| 2) | one | − | always large number of |
| | Staphylococcus | | variants (soup) |
| | aureus niche | | |
| 3) | multi | + | any smaller # of variants = |
| | Staphylococcus | | # of discrete niches |
| | aureus niches | | |
| 4) | multi | − | always large number of |
| | Staphylococcus | | variants (soup) |
| | aureus niches | | |

In Table 1, cases 2 & 4 can be eliminated, because co-colonization occurs in under 5% (in literature), and even in these cases the vast majority of co-colonization instances observed involve only one other organism. Case 3 can be considered as possible in a low number of cases (less than 5%) potentially relating to incomplete or non-overlapping footprints of the niche vs replacement organism.

There is no direct evidence from the literature as to whether the observed replacement of one variant for another (e.g. acquisition of MRSA) is caused by a biome-disruptive event or from a slow competitive replacement. However, it is empirically clear that only one strain at a time tends to colonize any individual biome (in toto). Biogeographically distinct and distant sites within a given biome strongly tend to have the same variant, and this occurs without any observable total body decolonization and replacement process, indicating that a rule-driven competitive replacement process occurs. The observation of competitive replacement is another expression of the principle of non-co-colonization.

In hypothetical cases where the replacement variant does not fill the niche completely there may be a weak tendency to co-colonization. In these cases, a variant cluster may be used to “fill the slots” with alternatives so that the co-colonization favors a synthetic replacement microorganism rather than the original pathogen. While this may involve the use of a different replacement microorganism, this is not recurrence—this is further blocking of recurrence.

Current Evidence of Non-Co-Colonization

One large study looked at the prevalence of co-colonization in 3,197 positive Staphylococcus aureus samples taken from healthy patients in Oxfordshire, England followed longitudinally for up to two years; the point prevalence of having multiple strains of Staphylococcus aureus in nares samples was 3.4 to 5.8%. Votintseva et al., 2014 J Clin Microbiol, 52 (4): 1192-1200. Of the Staphylococcus aureus carriers who submitted swabs nearly every two months for two years, 11% had transient co-carriage. The study used an effective spa typing protocol that allowed for a sensitive procedure for finding even low proportion co-colonization strains. The interpretation of this data set shows that Staphylococcus aureus colonization is a dynamic process with low prevalence of multiple Staphylococcus aureus strains vying for presence in the same niche over time. A simple calculation can establish that the observed results are not simply the independent occupation of a non-specific niche. In this instance, 1000 patients were screened and 360 were found to be Staphylococcus aureus positive. In a non-specific niche scenario, 0.36×0.36, or 13%, (130 persons), would be expected to display co-colonization; however only 3.9% of the 360 carriers, (14 persons) at that primary point were in fact co-colonized, demonstrating the strain specificity of the microbiome niche for Staphylococcus aureus.

A small percentage of Staphylococcus aureus carriers may be transiently colonized with two different strains of Staphylococcus aureus at any incident time point. As discussed above, Votintseva et al, looked at all variants within MSSA and MRSA and reported point incidences of this phenomenon to be in the range of 3.4-5.8%. The paper looking only at mixtures of MRSA and MSSA (would only find species that differ at the mecA site) is predictably lower at 2.3%. If co-colonization was a stable state, mixtures of Staphylococcus aureus species would be expected in virtually all samples. This is not observed.

Another study looked at 680 patients presenting for any type of hospital admission. It was practice of the National Health Service at that time to screen all patients being admitted for MRSA. Dall'Antonia, M. et al., 2005, J Hospital Infect 61, 62-67. During this evaluation the protocol was refined to discover MSSA, MRSA and co-colonized MRSA and MSSA patients. MSSA alone was found in 115 patients (16.9%), MRSA alone was found in 56 patients (8.2%) and co-colonization was discovered in 4 patients (0.58%), again supporting the view of a strain-exclusive niche in the microbiome for Staphylococcus aureus. It supports the concept that one Staphylococcus aureus strain can prevent the establishment of another. The results suggested a lower percentage of co-colonized carriers as would be predicted by the null hypothesis indicating that there is a significant protective effect against one Staphylococcus aureus strain colonization by a previous occupying resident Staphylococcus aureus strain. The statistical significance was p<0.01. The protective effect of MSSA colonization against MRSA colonization was calculated to be 78% (CI: 29-99%).

A further study looked at non-concordant Staphylococcus aureus isolates in a population composed of HIV infected IV drug users in a methadone clinic. There were 121 baseline positive Staphylococcus aureus samples and 4 of these showed clear discordance among 3 colonies evaluated by PFGE. However, re-evaluation of these 4 samples showed that 2 of the 4 were concordant at second evaluation. No discordance was found after re-evaluating 18 samples first found to be concordant. Therefore 1.7-3.3% of this population was found to have co-colonization at a singular time point. Cespedes C. et al., J Infect Dis 2005; 191: 444-52.

Historical Evidence of decolonization/recolonization studies also show evidence of Non-Co-Colonization. This principle has been previously partially demonstrated during the 1960s and 1970s in the well-known 80/81 to 502a “bacterial interference” studies and clinical applications. Absence of co-colonization is shown in the early bacterial interference papers in the 1960s and 1970s, these papers also clearly demonstrate “competitive exclusion” in regulating co-colonization. Mixed cultures of both 80/81 as the resident strain and 502A as the donor strain were not observed, experimentally demonstrating non-co-colonization as a stable situation for the microbiome. (Shinefield et al., 1963; Shinefield at al., 1966; Shinefield et al., 1973; Aly et al., 1974; Boris et al., 1964; Light et al., 1967; Fine et al., 1967).

Without “non-equivalence” and “competitive exclusion”, microbiome niches would consistently be filled with multiple strains of the same species of bacteria. The isolation in nature of a pure strain culture of Staphylococcus aureus from the nares would be a rare event if ever seen. The population dynamic in such a state would create a heterogeneous “soup” of many varieties of Staphylococcus aureus, as dictated by adventitious or random exposure from the environment. Any strain that the host has ever come in to contact with would have equal opportunity to colonize that space without competition or interference with any other strain variant (polyclonal colonization). The absence of this empirical result demonstrates “competitive exclusion”.

Yet, the exclusion principle is not so rigid that once a niche is occupied no other variant can usurp its position. These observations demonstrate an exclusion principle that is robust, but that allows external species to challenge an occupying species by briefly sharing that niche while the ultimate competition for dominance in that space is being enacted. On some occasions “new” strains overcome the previous resident strain and establish a new dominant resident strain. On other occasions, the interloper is rebuffed and the resident strain repels the attempt at replacement and reestablishes singular dominance. In both of these scenarios, the co-colonized state is transient and unstable; present at a low frequency.

Microbiomic Systems

Methods and compositions are provided to durably and safely prevent recurrence of a pathogenic microbial infection in a subject, comprising suppression of a pathogenic microorganism, replacement with a synthetic microorganism capable of occupying the same niche to durably exclude the pathogenic microorganism, and promotion of the synthetic microorganism for durable residence within the niche. This method is termed the BioPlx® method, as discussed above. In some embodiments, the subject is found to be colonized with the pathogenic microorganism prior to the suppression step.

In order to successfully work within the microbiome to promote the colonization of a desired organism in such a way as to produce a durable protective outcome requires that we know the “rules” of microbiomes: as discussed in greater detail in the sections following.

A non-co-colonization model has been developed to provide context and establish target product characteristics. The rationale for the present technology rests on the Microbiomic paradigm (biome/ecosystem/niche), and on the Microbiome having certain persistent and verifiable characteristics. The key discoverable metric rests on co-colonization statistics in literature modified by specifics on decolonization, testing, and other relevant conditions, followed by direct observations from the clinical study of example 1.

The skin microbiome in the subject is an entity, a persistent identifiable thing. Over 10,000 different species of microorganisms make up the skin microbiome. The skin biome is an ecosystem which may be defined as a system, or group of interconnected elements, formed by the interaction of a community of organisms with their environment. The skin microbiome ecosystem has a “healthy”, or “normal” base state. The biome can be “healthy” or “sick” (dysbiosis), and can be invaded by pathogenic organisms—in other words the Microbiome can be invaded by a “Bad Bug”—such as MRSA—it can also become infected or contaminated by undesirable organisms or variants (dysbiosis). Dysbiosis is a term for a microbial imbalance or maladaptation on or inside the body, such as an impaired microbiota.

The skin microbiome has a structure created by a vast combinatorial web of relationships between the host and all of the components of the biome. The microbiome, or biome, is a dynamically structured complex system and is an “elastically resilient” ecosystem.

The skin microbiome has a dynamic but persistent structure—it is “resilient”, for example, even under conditions of massive cell death (e.g. washing, using ethanol, hand sanitizer, etc.) the biome regenerates in a similar form.

Resilience

The human microbiome has the quality of resilience meaning that mild perturbations tend to re-correct toward a previous established baseline of species mixture and concentration. However, members of each niche can be successfully challenged for their place in that stable mixture either as a result of an acute external disruptive event (i.e. an antimicrobial medication or an antiseptic application) or as a slow competitive replacement.

In ecology, resilience is the capacity of an ecosystem to respond to a perturbation or disturbance by resisting damage and recovering quickly. Resilience refers to ecosystem's stability and capability of tolerating disturbance and restoring itself.

In the literature, the main mathematical definitions of resilience are based on dynamical systems theory, and more specifically on attractors and attraction basins. The human microbiome operates in many ways like a multi-basin complex system. It changes states or basins, but then resilience stabilizes that state. Martin, S. et al., 2011, in: Deffuant G., Gilbert N. (eds) Viability and Resilience of Complex Systems. Understanding Complex Systems. Springer, Berlin, Heidelberg, pp. 15-36.

The microbiome operates in many ways like a multi-attractor complex system—it can changes its states or basins, but then the resilience associated with that attractor stabilizes that state.

Ecological resilience is defined as the capacity of a system to absorb disturbance and reorganize while undergoing change so as to still retain essentially the same function, structure, identity and feedbacks. Mitra, C., et al., 2015, An integrative quantifier of multistability in complex systems based on ecological resilience, Nature, Scient. Rep., 5, 1-12.

The “competitive exclusion principle” provides that complete competitors cannot exist. The “axiom of inequality” states that no two things or processes in a real world are precisely equal. Hardin, 1960, Science, vol. 131, 1292-1297, p. 1292. Based on Hardin's ‘Axiom of Inequality’ and the Competitive Exclusion Principle, long-term durability should only be achieved by close variant substitution, but would not likely be available with respect to species substitution. For example, MRSA and MSSA can co-colonize briefly—just like any other variants of Staphylococcus aureus can co-colonize in transient fashion. See Dall'Antonia, M. et al., 2005, J Hospital Infect 61, 62-67, disclosing a study of 680 patients presenting for any type of hospital admission and screened all patients being admitted for MRSA. During this evaluation the protocol was refined to discover MSSA, MRSA and co-colonized MRSA and MSSA patients. MSSA alone was found in 115 patients (16.9%), MRSA alone was found in 56 patients (8.2%) and co-colonization was discovered in 4 patients (0.58%), again supporting the view of a strain-exclusive niche in the microbiome for Staphylococcus aureus. It supports the concept that one Staphylococcus aureus strain can prevent the establishment of another.

Resilience may create recurrence—an observed natural phenomenon—as the existing (MRSA contaminated) biome tries to preserve itself.

However, resilience can also prevent MRSA recurrence—as exhibited by methods and compositions provided herein. By suppressing a pathogenic microorganism such as MRSA (“bad bug”) colonized in a subject, and replacing with a safe synthetic microorganism (“good bug”) of the same species, it has been established that the “good bug” durably prevents recurrence of the “bad bug” (prevents MRSA re-invasion).

A historical example of resilience creating durable, persistent substitution is seen in Staphylococcus aureus carriers and replacement with strain 502a. Aly et al., 1974 J Infect Dis 129(6) pp. 720-724, studied bacterial interference in carriers of Staphylococcus aureus. The carriers were treated with antibiotics and antibacterial soaps and challenged with Staphylococcus aureus strain 502a. It was found that full decolonization was needed to get good colonization of 502a. Day 7 showed 100% take, but at day 23 the take was down to 60 to 80%. The persistence data was 73% at 23 weeks for well-decolonized subjects. Thus, long-term durability is only achieved by close variant substitution. Commensal microflora (normal microflora, indigenous microbiota) can help recolonization dynamics, but they do not fulfill close variant durability requirements. The inventors have designed a method for obtaining a “passive” version of an organism or pathogen (same species) that is to be “replaced” or “excluded”.

A relative stability in the microbial ecosystem of adults in the absence of gross perturbation has been suggested, and that long-term stability of human communities is not maintained by inertia, but by the action of restoring forces within a dynamic system. Relman, D. A., 2012, Nutr Rev., 70 (Suppl 1): S2-S9.

Functional resilience is an intrinsic property of microbial communities and it has been suggested that state changes in response to environmental variation may be a key mechanism driving functional resilience in microbial communities. Song et al., 2015, Frontiers in Microbiology, 6, 1298. Seeking an integrated concept applicable to all microbial communities, Song et al. compared engineering and ecological resilience and reconciled them by arguing that resilience is an intrinsic property of complex adaptive systems which, after perturbation, recover their system-level functions and interactions with the environment, rather than their endogenous state.

Thus, a biome ecosystem has a dynamic but “stable elastoplastic equilibrium”. Once perturbed the biome “tries” to return to equilibrium. At any given moment the biome ecosystem has an equilibrium “base state”. Even under conditions of stress or massive cell death (e.g. washing, using ethanol, hand sanitizer, etc.) the biome is observed to typically regenerate in a similar form.

Microbiome ecosystems have “niches” defined by structure and internal and external interactions. One “fact” or “principal” of any biome structure is that different organisms occupy different “niches” in the biome, as defined/allowed by the structure of relationships. An ecological “niche” is the role and position a species has in its environment; how it meets its needs for food and shelter, how it survives, and how it reproduces. A species' niche includes all of its interactions with the biotic and abiotic factors of its environment. A biome “niche” has specific environmental factors and conditions including, for example, pH, temperature, osmotic pressure, osmolality, oxygen level, nutrient concentration, blood concentration, plasma concentration, serum concentration, and electrolyte concentration.

Different organisms occupy different “niches” in the biome, as defined/allowed by the relationships structure. Niches as durable features of the biome ecosystem. Each niche has boundary conditions; a virtual shape or “footprint” reflecting the shape, which is discussed in the context of the “Hutchinsonian niche”.

The Hutchinsonian niche is an n-dimensional hypervolume, where the dimensions are environmental conditions and resources, that define the requirements of an individual or a species to practice “its” way of life, more particularly, for its population to persist. The “hypervolume” defines the multi-dimensional space of resources (e.g., light, nutrients, structure, etc.) available to (and specifically used by) organisms, and “all species other than those under consideration are regarded as part of the coordinate system.”

A niche is a very specific segment of ecospace occupied by a single species. On the presumption that no two species are identical in all respects (i.e., Hardin's ‘axiom of inequality’) and the competitive exclusion principle, some resource or adaptive dimension will provide a niche specific to each species.

Niches are exclusive. Each organism competes with similar organisms for that niche, and the successful organism fills that niche. Two organisms do not/cannot fill the same niche because one will out-compete the other over time. Therefore, the coexistence of two organisms in the same biome over extended time periods means they do not fill the same niche.

Once a niche is left vacant, other organisms can fill that position. This is because one species does not have the same footprint as another species, so one species cannot fill the same niche as another species. Successful replacement requires that the same organism (e.g., same species or close variant) should be used to fill or durably replace within a niche. It is recognized that partial competition exists in the form of transient colonization/infection and is an observable phenomenon.

Partial competition for a single niche can occur. One organism can “narrow” the “niche width” of another by partial competition. This might be the case with Staphylococcus epidermidis vs. Staphylococcus aureus. S. epidermidis is a commensal bacterium that secretes a serine protease capable of disassembling preformed Staphylococcus aureus biofilms, when used in high enough concentrations. Sugimoto et al., J Bacteriol, 195(8) 1645-1655. However, there is an important distinction between an organism as a carrier of a toxic phenotypic expression (being temporarily massively overloaded by application at a site), vs that organism as a durable inhabitant of a niche that narrows or outcompetes the pathogen.

Interspecies co-colonization is a different phenomenon than the ability to durably fill and block an ecological niche. For example, Shu et al., 2013 demonstrate that fermentation of glycerol to form short chain fatty acids (SCFA) with Cutibacterium acnes (C. acnes), a skin commensal bacterium that can inhibit growth of USA300, the most prevalent community-acquired methicillin-resistant Staphylococcus aureus (CA-MRSA). Shu demonstrates that SCFAs produced by C. acnes under anaerobic conditions inhibits Staphylococcus aureus growth in high concentrations. Shu et al., 2013 PLoS ONE 8(2): e55380. However, these bacteria and this fermentation capability of C. acnes are already present in the normal human skin biome without there being effective eradication or diminution of Staphylococcus aureus pathogenicity. There is not any reason to believe that a hyper-physiologic application of these substrates would accomplish the goal of reduction of Staphylococcus aureus colonization or incidence of disease.

Decolonization/Recolonization

A method is provided to treat, prevent, or prevent recurrence of mastitis or intramammary infection caused by a pathogenic microorganism in a cow, goat or sheep. A method is provided to prevent or decrease recurrence of a pathogenic infection of a undesirable microorganism in a bovine, ovine, or caprine subject, comprising the steps of (i) suppressing (decolonizing) the undesirable microorganism on at least one site in the subject to reduce or eliminate the presence of the undesirable microorganism from the site; and (ii) replacing the undesirable microorganism by administering to the subject at the at least one site a synthetic second microorganism having the same genus and species as the undesirable microorganism. Optionally, the method further comprises (iii) promoting colonization of the synthetic microorganism, for example, at the site of administration.

In some embodiments, the undesirable microorganism is a pathogenic microorganism and the term suppress (S) refers to a process of suppressing, reducing or eliminating the pathogenic microorganism at one or more, two or more, three or more, four or more sites in a subject. For example, the undesirable microorganism may be subject to nasal, mucosal, and/or dermal decolonization protocols.

The term replace (R) refers to replacing the pathogenic microorganism with a synthetic microorganism that is benign, drug-susceptible, and/or incapable of causing systemic or pathogenic infection in the subject. The replacement microorganism may be a molecularly modified synthetic microorganism of the same species as the pathogenic microorganism. The synthetic microorganism may be a molecularly modified microorganism of the same species, different strain, as the pathogenic microorganism, such that the synthetic microorganism is able to colonize the site on the subject, but is unable to cause systemic infection in the subject. By filling the vacated niche of the pathogenic microorganism, the synthetic microorganism is able to eliminate re-colonization by the pathogenic microorganism in the subject and thereby decrease or eliminate recurrence of pathogenic infection.

The term promote (P) refers to methods and compositions to promote replacement synthetic microorganism in the subject, for example, by employing prebiotics and biome management, for example, by employing a biome modulator in order to promote and support the new biome comprising the synthetic microorganism.

These methods broadly define a platform technology (SRP), with specifically designed protocols developed to address specific medical conditions (e.g. Staph aureus, MRSA). If the processes of S, R, and P are selected properly—opening and then filling and sustaining a specific biome niche—a “durable” persistent biome is created that is capable of repelling pathogenic colonization.

A method is provided to decrease recurrence or chance of systemic infection of a pathogenic microorganism in a subject, the method comprising suppressing the pathogenic microorganism on the subject to significantly reduce or eliminate the detectable presence of the pathogenic microorganism; and replacing the pathogenic microorganism by administering a synthetic microorganism to the subject, wherein the synthetic microorganism is capable of occupying the same niche as the pathogenic microorganism as evidenced by (1) having the same genetic background, or genus and species, as the pathogenic microorganism, and/or by (2) exhibiting durable detectable presence on the subject for at least 60 days following replacement. The method may include promoting the colonization of the synthetic microorganism on at least one site in the subject. In some cases, the subject may have been found to be colonized by the pathogenic microorganism.

Frequently, systemic infection of a bovine, ovine, or caprine subject with a pathogenic microorganism may be preceded by colonization of the pathogenic microorganism in the subject. For example, a substantial proportion of cases of Staphylococcus aureus bacteremia in humans appear to be of endogenous origin since they may originate from colonies in the nasal mucosa. For example, in one multicenter study of Staphylococcus aureus bacteremia, the blood isolates were identical to those from the anterior nares in 180 of 219 patients (82.2%). In a second study, 14 of 1278 patients who had nasal colonization with Staphylococcus aureus subsequently had Staphylococcus aureus bacteremia. In 12 of these 14 patients (86%), the isolates obtained from the nares were clonally identical to the isolates obtained from blood 1 day to 14 months later. See von Eiff et al., 2001, NEJM, vol. 344, No. 1, 11-16. Another study showed the relative risk of Staphylococcus aureus bacteremia was increased multi-fold in nasal carriers when compared to non-carriers, reporting an 80% match between the invasive isolate and previously found colonizing strain. Wertheim et al., Lancet 2004; 364: 703-705.

In some embodiments, the subject is found to be colonized with the pathogenic strain of the microorganism prior to systemic infection. In other embodiments, the subject may have been colonized or infected by a nosocomial (hospital-acquired) strain or community-acquired strain of a pathogenic microorganism.

The pathogenic microorganism may be a wild-type microorganism, and/or a pathogenic microorganism that may be colonized or detectably present in at least one site in the subject. The site may be a dermal or mucosal site in the subject. The one or sites of colonization may include intramammary sites and/or extramammary sites. Sites of colonization may include teat canal, teat cistern, gland cistern, streak canal, teat apices, teat skin, udder skin, perineum skin, rectum, vagina, muzzle area, nares, and oral cavity. Sites may be identified by swab samples. In addition, hands of human herd staff, nares of human herd staff, equipment, water buckets, calf bottles, mangers, bedding, housing, and teat cups or equipment may be reservoirs. Roberson et al., 1994, J Dairy Sci, 77:3354-3364.

In humans, for example, the site may include soft tissue including, but are not limited to, nares, throat, perineum, inguinal region, vagina, nasal, groin, perirectal area, finger webs, forehead, pharynx, axillae, hands, chest, abdomen, head, and/or toe webs.

The pathogenic microorganism may be a drug resistant microorganism. The Centers for Disease Control (CDC) recently published a report outlining the top 18 drug-resistant threats to the United States, see www.cdc.gov/drugresistance/biggest_threats. In some embodiments, the undesirable microorganism is selected from Neisseria gonorrhoeae, fluconazole-resistant Candida, methicillin-resistant Staphylococcus aureus (MRSA), vancomycin-resistant Staphylococcus aureus, drug-resistant Streptococcus pneumoniae and drug-resistant tuberculosis, erythromycin-resistant Group A Streptococcus, and clindamycin-resistant Group B Streptococcus.

In some embodiments, the pathogenic microorganism is a MRSA.

The synthetic microorganism (a) must be able to fill the ecological niche in the at least one site in the subject so as to durably exclude the undesirable microorganism following suppression; and (b) must have at least one molecular modification comprising a first cell death gene operably linked to a first regulatory region comprising a first promoter that is activated (induced) by a change in state in the environment compared to the normal physiological conditions in at least one site in the subject.

The synthetic microorganism may be of the same genus and species as the undesirable microorganism, in order to enhance the ability to fill the niche and durably exclude the undesirable microorganism in at least one site in the subject.

In some embodiments, the disclosure provides a synthetic microorganism that is not a pathogen and cannot become an accidental pathogen because it does not have the ability to infect the subject upon change in state, e.g., upon exposure to blood or serum. The synthetic microorganism comprises at least one molecular modification comprising a first cell death gene operably linked to a first regulatory region comprising a first promoter that is activated (induced) by a change in state in the environment compared to the normal physiological conditions in at least one site in the subject. For example, if the site in the subject is a dermal or mucosal site, then exposure to blood or serum is a change in state resulting in cell death of the synthetic microorganism. For example, average cell death of the synthetic microorganism may occur within 6 hours, 5 hours, 4 hours, 2 hours, 90 minutes, 60 minutes, 45 minutes, 30 minutes, 20 minutes, 15 minutes, 10 minutes, 5 minutes, 2 minutes or 1 minute following change of state. The change in state may be a change in one or more of the following conditions: pH, temperature, osmotic pressure, osmolality, oxygen level, nutrient concentration, blood concentration, plasma concentration, serum concentration, and/or electrolyte concentration from that in at least one site in a subject. In some embodiments, the change in state is a higher concentration of blood, serum, or plasma compared to normal physiological conditions at the at least one site in the subject.

In one embodiment, the pathogenic microorganism is a MRSA. MRSA is a variant subgroup of Staphylococcus aureus. MRSA strains typically include a mecA cassette that allows production of an alternate penicillin binding protein that render them resistant to treatment with most beta-lactam and other first-line antibiotics. Staphylococcus aureus as a whole (including MRSA) is present as part of the normal microbiome of approximately 30% of the total human population. As part of the microbiome Staphylococcus aureus lives most commonly on the surface of the skin and in the anterior nasal vestibules, but can also be found in smaller amounts in the deep oropharynx and gastrointestinal tract and as part of the normal vaginal flora in some individuals.

In the majority of individuals Staphylococcus aureus remains a non-invasive commensal bacterium merely occupying an ecologic niche and not causing disease. Human herd managers or handlers may serve as a reservoir for cows, goats, or sheep. The colonization state is far more common than that of invasive disease—some researchers estimate this ratio to be on the order of 1000 to one. Laupland et al., J Infect Dis (2008) 198:336. However, in a fraction of those colonized this bacterium can cause disease either opportunistically or as a result of increased tendencies toward invasion due to the acquisition of genetic cassettes coding for virulence protein products that allow such strains to more effectively invade through the epidermal or mucosal tissue layers initiating deep infection. In both above circumstances, the presence of the mecA cassette limits the treatment options for these patients and a number of studies have documented the increased mortality rate associated with MRSA when compared to MSSA in bacteremia, endovascular infection and pneumonia.

### Definitions

The singular forms “a”, “an” and “the” are intended to include the plural forms as well, unless the context clearly indicates otherwise.

The term “and/or” refers to and encompasses any and all possible combinations of one or more of the associated listed items.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The term “pathogen” or “pathogenic microorganism” refers to a microorganism that is capable of causing disease. A pathogenic microorganism may colonize a site on a subject and may subsequently cause systemic infection in a subject. The pathogenic microorganism may have evolved the genetic ability to breach cellular and anatomic barriers that ordinarily restrict other microorganisms. Pathogens may inherently cause damage to cells to forcefully gain access to a new, unique niche that provides them with less competition from other microorganisms, as well as with a ready new source of nutrients. Falkow, Stanley, 1998 Emerging Infectious Diseases, Vol. 4, No. 3, 495-497. The pathogenic microorganism may be a drug-resistant microorganism.

The term “virulent” or “virulence” is used to describe the power of a microorganism to cause disease.

The term “commensal” refers to a form of symbioses in which one organism derives food or other benefits from another organism without affecting it. Commensal bacteria are usually part of the normal flora.

The term “suppress” or “decolonize” means to substantially reduce or eliminate the original undesired pathogenic microorganism by various means (frequently referred to as “decolonization”). Substantially reduce refers to reduction of the undesirable microorganism by greater than 90%, 95%, 98%, 99%, or greater than 99.9% of original colonization by any means known in the art.

The term “replace” refers to replacing the original pathogenic microorganism by introducing a new microorganism (frequently referred to as “recolonization”) that “crowds out” and occupies the niche(s) that the original microorganism would ordinarily occupy, and thus preventing the original undesired microorganism from returning to the microbiome ecosystem (frequently referred to as “interference” and “non-co-colonization”).

The term “durably replace”, “durably exclude”, “durable exclusion”, or “durable replacement”, refers to detectable presence of the new synthetic microorganism for a period of at least 30 days, 60 days, 84 days, 120 days, 168 days, or 180 days after introduction of the new microorganism to a subject, for example, as detected by swabbing the subject. In some embodiments, “durably replace”, “durably exclude”, “durable exclusion”, or “durable replacement” refers to absence of the original pathogenic microorganism for a period of at least 30 days, 60 days, 84 days, 120 days, 168 days, or 180 days after introduction of the new synthetic microorganism to the subject, for example, absence as detected over at least two consecutive plural sample periods, for example, by swabbing the subject.

The term “rheostatic cell” refers to a synthetic microorganism that has the ability to durably occupy a native niche, or naturally occurring niche, in a subject. The rheostatic cell also has the ability to respond to change in state in its environment.

The term “promote”, or “promoting”, refers to activities or methods to enhance the colonization and survival of the new organism, for example, in the subject. For example, promoting colonization of a synthetic bacteria in a subject may include administering a nutrient, prebiotic, and/or probiotic bacterial species.

The terms “prevention”, “prevent”, “preventing”, “prophylaxis” and as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The disclosure provides methods and compositions comprising a synthetic microorganism useful for eliminating and preventing the recurrence of a undesirable microorganism in a subject hosting a microbiome, comprising (a) decolonizing the host microbiome; and (b) durably replacing the undesirable microorganism by administering to the subject the synthetic microorganism comprising at least one element imparting a non-native attribute, wherein the synthetic microorganism is capable of durably integrating to the host microbiome, and occupying the same niche in the host microbiome as the undesirable microorganism.

In some embodiments, a method is provided comprising a decolonizing step comprising topically administering a decolonizing agent to at least one site in the subject to reduce or eliminate the presence of an undesirable microorganism from the at least one site.

In some embodiments, the decolonizing step comprises topical administration of a decolonizing agent, wherein no systemic antimicrobial agent is simultaneously administered. In some embodiments, no systemic antimicrobial agent is administered prior to, concurrent with, and/or subsequent to within one week, two weeks, three weeks, one month, two months, three months, six months, or one year of the first topical administration of the decolonizing agent or administration of the synthetic microorganism. In some embodiments, the decolonizing agent is selected from the group consisting of a disinfectant, bacteriocide, antiseptic, astringent, and antimicrobial agent.

The disclosure provides a synthetic microorganism for durably replacing an undesirable microorganism in a subject. The synthetic microorganism comprises a molecular modification designed to enhance safety by reducing the risk of systemic infection. In one embodiment, the molecular modification causes a significant reduction in growth or cell death of the synthetic microorganism in response to blood, serum, plasma, or interstitial fluid. The synthetic microorganism may be used in methods and compositions for preventing or reducing recurrence of dermal or mucosal colonization or recolonization of an undesirable microorganism in a subject.

The disclosure provides a synthetic microorganism for use in compositions and methods for treating or preventing, reducing the risk of, or reducing the likelihood of colonization, or recolonization, systemic infection, bacteremia, or endocarditis caused by an undesirable microorganism in a subject.

In some embodiments, the subject treated with a method according to the disclosure does not exhibit recurrence or colonization of an undesirable microorganism as evidenced by swabbing the subject at the at least one site for at least two weeks, at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any human or food chain mammal, such as cattle (e.g., cows), goats, sheep, camel, yak, buffalo, horse, donkey, zebu, reindeer, giraffe, or swine (e.g., sows). In some embodiments, the subject may be a human subject. In particular, the term may specify male or female. In one embodiment, the subject is a female cow, goat, or sheep. In another embodiment, both female and male animals may be subjects to reduce chances of pathogen reservoirs. In one aspect, the patient is an adult animal. In another aspect, the patient is a non-neonate animal. In another aspect, the subject is a heifer, lactating cow, or dry cow. In some embodiments, the subject is a female or male human handler or herd manager found to be colonized with a pathogenic strain of a microorganism.

The term “neonate”, or newborn, refers to an infant in the first 28 days after birth. The term “non-neonate” refers to an animal older than 28 days.

The term “effective amount” as used herein refers to an amount of an agent, either alone or as a part of a pharmaceutical composition, that is capable of having any detectable, positive effect on any symptom, aspect, or characteristics of a disease state or condition. Such effect need not be absolute to be beneficial.

The term “measurable average cell death” refers to the inverse of survival percentage for a microorganism determined at a predefined period of time after introducing a change in state compared to the same microorganism in the absence of a change in state under defined conditions. The survival percentage may be determined by any known method for quantifying live microbial cells. For example, survival percentage may be calculated by counting cfus/mL for cultured synthetic microorganism cells and counting cfus/mL of uninduced synthetic microorganism cells at the predefined period of time, then dividing cfus induced/mL by cfus/mL uninduced×100=x % survival percentage. The measurable average cell death may be determined by 100%−x % survival percentage=y % measurable average cell death. For example, wherein the survival percentage is 5%, the measurable average cell death is 100%-5%=95%. Any method for counting cultured live microbial cells may be employed for calculation of survival percentage including cfu, OD600, flow cytometry, or other known techniques. Likewise, an induced synthetic strain may be compared to a wild-type target microorganism exposed to the same conditions for the same period of time, using similar calculations to determine a “survival rate” wherein 100%-survival rate=z % “reduction in viable cells”.

In some embodiments, the change in state is a change in the cell environment which may be, for example, selected from one or more of pH, temperature, osmotic pressure, osmolality, oxygen level, nutrient concentration, blood concentration, plasma concentration, serum concentration, metal concentration, iron concentration, chelated metal concentration, change in composition or concentration of one or more immune factors, mineral concentration, and electrolyte concentration. In some embodiments, the change in state is a higher concentration of and/or change in composition of blood, serum, plasma, cerebral spinal fluid (CSF), contaminated CSF, synovial fluid, or interstitial fluid, compared to normal physiological (niche) conditions at the at least one site in the subject. In some embodiments, “normal physiological conditions” may be dermal or mucosal conditions, or cell growth in a complete media such as TSB.

The term “including” as used herein is non-limiting in scope, such that additional elements are contemplated as being possible in addition to those listed; this term may be read in any instance as “including, but not limited to.”

The term “shuttle vector” as used herein refers to a vector constructed so it can propagate in two different host species. Therefore, DNA inserted into a shuttle vector can be tested or manipulated in two different cell types.

The term “plasmid” as used herein refers to a double-stranded DNA, typically in a circular form, that is separate from the chromosomes, for example, which may be found in bacteria and protozoa.

The term “expression vector”, also known as an “expression construct”, is generally a plasmid that is used to introduce a specific gene into a target cell.

The term “transcription” refers to the synthesis of RNA under the direction of DNA.

The term “transformation” or “transforming” as used herein refers to the alteration of a bacterial cell caused by transfer of DNA. The term “transform” or “transformation” refers to the transfer of a nucleic acid fragment into a parent bacterial cell, resulting in genetically-stable inheritance. Synthetic bacterial cells comprising the transformed nucleic acid fragment may also be referred to as “recombinant” or “transgenic” or “transformed” organisms.

As used herein, “stably maintained” or “stable” synthetic bacterium is used to refer to a synthetic bacterial cell carrying non-native genetic material, e.g., a cell death gene, and/or other action gene, that is incorporated into the cell genome such that the non-native genetic material is retained, and propagated. The stable bacterium is capable of survival and/or growth in vitro, e.g., in medium, and/or in vivo, e.g., in a dermal, mucosal, or other intended environment.

The term “operon” as used herein refers to a functioning unit of DNA containing a cluster of genes under the control of a single promoter. The genes are transcribed together into an mRNA strand and either translated together in the cytoplasm, or undergo splicing to create monocistronic mRNAs that are translated separately, i.e. several strands of mRNA that each encode a single gene product. The result of this is that the genes contained in the operon are either expressed together or not at all. Several genes must be co-transcribed to define an operon.

The term “operably linked” refers to an association of nucleic acid sequences on a single nucleic acid sequence such that the function of one is affected by the other. For example, a regulatory element such as a promoter is operably linked with an action gene when it is capable of affecting the expression of the action gene, regardless of the distance between the regulatory element such as the promoter and the action gene. More specifically, operably linked refers to a nucleic acid sequence, e.g., comprising an action gene, that is joined to a regulatory element, e.g., an inducible promoter, in a manner which allows expression of the action gene(s).

The term “regulatory region” refers to a nucleic acid sequence that can direct transcription of a gene of interest, such as an action gene, and may comprise various regulatory elements such as promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

The term “promoter” or “promoter gene” as used herein refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. In some cases, promoters may regulate expression of a coding sequence or gene in response to a particular stimulus, e.g., in a cell- or tissue-specific manner, in response to different environmental or physiological conditions, or in response to specific compounds. Prokaryotic promoters may be classified into two classes: inducible and constitutive.

An “inducible promoter” or “inducible promoter gene” refers to a regulatory element within a regulatory region that is operably linked to one or more genes, such as an action gene, wherein expression of the gene(s) is increased in response to a particular environmental condition or in the presence of an inducer of said regulatory region. An “inducible promoter” refers to a promoter that initiates increased levels of transcription of the coding sequence or gene under its control in response to a stimulus or an exogenous environmental condition. The inducible promoter may be induced upon exposure to a change in environmental condition. The inducible promoter may be a blood or serum inducible promoter, inducible upon exposure to a protein, inducible upon exposure to a carbohydrate, or inducible upon a pH change.

The blood or serum inducible promoter may be selected from the group consisting of isdB, leuA, hlgA, hlgA2, isdG, sbnC, sbnE, hlgB, SAUSA300_2616, splF, fhuB, hlb, hrtAB, IsdG, LrgA, SAUSA300_2268, SAUSA200_2617, SbnE, IsdI, LrgB, SbnC, HlgB, IsdG, SplF, IsdI, LrgA, HlgA2, CH52_04385, CH52_05105, CH52_06885, CH52_10455, PsbnA, and sbnA.

The term “constitutive promoter” refers to a promoter that is capable of facilitating continuous transcription of a coding sequence or gene under its control and/or to which it is operably linked under normal physiological conditions.

The term “animal” refers to the animal kingdom definition.

The term “substantial identity” or “substantially identical,” when referring to a nucleotide or fragment thereof, indicates that, when optimally aligned with appropriate nucleotide insertions or deletions with another nucleotide (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleotide molecule having substantial identity to a reference nucleotide molecule may, in certain instances, encode a polypeptide having the same or substantially similar amino acid sequence as the polypeptide encoded by the reference nucleotide molecule.

The term “derived from” when made in reference to a nucleotide or amino acid sequence refers to a modified sequence having at least 50% of the contiguous reference nucleotide or amino acid sequence respectively, wherein the modified sequence causes the synthetic microorganism to exhibit a similar desirable attribute as the reference sequence of a genetic element such as promoter, cell death gene, antitoxin gene, virulence block, or nanofactory, including upregulation or downregulation in response to a change in state, or the ability to express a toxin, antitoxin, or nanofactory product, or a substantially similar sequence, the ability to transcribe an antisense RNA antitoxin, or the ability to prevent or diminish horizontal gene transfer of genetic material from the undesirable microorganism. The term “derived from” in reference to a nucleotide sequence also includes a modified sequence that has been codon optimized for a particular microorganism to express a substantially similar amino acid sequence to that encoded by the reference nucleotide sequence. The term “derived from” when made in reference to a microorganism, refers to a target microorganism that is subjected to a molecular modification to obtain a synthetic microorganism.

The term “substantial similarity” or “substantially similar” as applied to polypeptides means that two peptide or protein sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions.

The term “conservative amino acid substitution” refers to wherein one amino acid residue is substituted by another amino acid residue having a side chain (R group) with similar chemical properties, such as charge or hydrophobicity. In general, a conservative amino acid substitution will not substantially change the functional properties of the, e.g., toxin or antitoxin protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Polypeptide sequences may be compared using FASTA using default or recommended parameters, a program in GCG Version 6.1. FASTA (e.g., FASTA2 and FASTA3) provides alignments and percent sequence identity of the regions of the best overlap between the query and search sequences (see, e.g., Pearson, W. R., Methods Mol Biol 132: 185-219 (2000), herein incorporated by reference). Another preferred algorithm when comparing a sequence of the disclosure to a database containing a large number of sequences from different organisms is the computer program BLAST, especially BLASTP or TBLASTN, using default parameters. See, e.g., Altschul et al., J Mol Biol 215:403-410 (1990) and Altschul et al., Nucleic Acids Res 25:3389-402 (1997).

Unless otherwise indicated, nucleotide sequences provided herein are presented in the 5′-3′ direction.

All pronouns are intended to be given their broadest meaning. Unless stated otherwise, female pronouns encompass the male, male pronouns encompass the female, singular pronouns encompass the plural, and plural pronouns encompass the singular.

The term “systemic administration” refers to a route of administration into the circulatory system so that the entire body is affected. Systemic administration can take place through enteral administration (absorption through the gastrointestinal tract, e.g. oral administration) or parenteral administration (e.g., injection, infusion, or implantation).

The term “topical administration” refers to application to a localized area of the body or to the surface of a body part regardless of the location of the effect. Typical sites for topical administration include sites on the skin or mucous membranes. In some embodiments, topical route of administration includes enteral administration of medications or compositions.

The term “undesirable microorganism” refers to a microorganism which may be a pathogenic microorganism, drug-resistant microorganism, antibiotic-resistant microorganism, irritation-causing microorganism, odor-causing microorganism and/or may be a microorganism comprising an undesirable virulence factor.

The “undesirable microorganism” may be selected from the group consisting of Staphylococcus aureus, coagulase-negative staphylococci (CNS), Streptococci Group A, Streptococci Group B, Streptococci Group C, Streptococci Group C & G, Staphylococcus spp., Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, and Pseudomonas aeruginosa.

In some embodiments, the undesirable microorganism is an antimicrobial agent-resistant microorganism. In some embodiments, the antimicrobial agent-resistant microorganism is an antibiotic resistant bacteria. In some embodiments, the antibiotic-resistant bacteria is a Gram-positive bacterial species selected from the group consisting of a Streptococcus spp., Cutibacterium spp., and a Staphylococcus spp. In some embodiments, the Streptococcus spp. is selected from the group consisting of Streptococcus pneumoniae, Streptococcus mutans, Streptococcus sobrinus, Streptococcus pyogenes, and Streptococcus agalactiae. In some embodiments, the Cutibacterium spp. is selected from the group consisting of Cutibacterium acnes subsp. acnes, Cutibacterium acnes subsp. defendens, and Cutibacterium acnes subsp. elongatum. In some embodiments, the Staphylococcus spp. is selected from the group consisting of Staphylococcus aureus, Staphylococcus epidermidis, and Staphylococcus saprophyticus. In some embodiments, the undesirable microorganism is a methicillin-resistant Staphylococcus aureus (MRSA) strain that contains a staphylococcal chromosome cassette (SCCmec types I-III), which encode one (SCCmec type I) or multiple antibiotic resistance genes (SCCmec type II and III), and/or produces a toxin. In some embodiments, the toxin is selected from the group consisting of a Panton-Valentine leucocidin (PVL) toxin, toxic shock syndrome toxin-1 (TSST-1), staphylococcal alpha-hemolysin toxin, staphylococcal beta-hemolysin toxin, staphylococcal gamma-hemolysin toxin, staphylococcal delta-hemolysin toxin, enterotoxin A, enterotoxin B, enterotoxin C, enterotoxin D, enterotoxin E, and a coagulase toxin.

In some embodiments, the undesirable microorganism is a Staphylococcus aureus strain, and wherein the detectable presence is measured by a method comprising obtaining a sample from at least one site of the subject, contacting a chromogenic agar with the sample, incubating the contacted agar and counting the positive cfus of the bacterial species after a predetermined period of time.

The term “synthetic microorganism” refers to an isolated microorganism modified by any means to comprise at least one element imparting a non-native attribute. For example, the synthetic microorganism may be engineered to include a molecular modification comprising an addition, deletion and/or modification of genetic material to incorporate a non-native attribute. In some embodiments, the synthetic microorganism is not an auxotroph.

The term “auxotroph”, “auxotrophic strain”, or “auxotrophic mutant”, as used herein refers to a strain of microorganism that requires a growth supplement that the organism from nature (wild-type strain) does not require. For example, auxotrophic strains of Staphylococcus epidermidis that are dependent on D-alanine for growth are disclosed in US 20190256935, Whitfill et al., which is incorporated herein by reference.

The term “biotherapeutic composition” or “live biotherapeutic composition” refers to a composition comprising a synthetic microorganism according to the disclosure.

The term “live biotherapeutic product” (LBP) as used herein refers to a biological product that 1) contains live organisms, such as bacteria; 2) is applicable to prevention, treatment, or cure of a disease or condition in human beings; and 3) is not a vaccine. As described herein, LBPs are not filterable viruses, oncolytic bacteria, or products intended as gene therapy agents, and as a general matter, are not administered by injection.

A “recombinant LBP” (rLBP) as used herein is a live biotherapeutic product comprising microorganisms that have been genetically modified through the purposeful addition, deletion, or modification of genetic material.

A “drug” as used herein includes but is not limited to articles intended for use in the diagnosis, cure, mitigation, treatment, or prevention of disease in man or other animals.

A “drug substance” as used herein is the unformulated active substance that may subsequently be formulated with excipients to produce drug products. The microorganisms contained in an LBP are typically cellular microbes such as bacteria or yeast. Thus the drug substance for an LBP is typically the unformulated live cells.

A “drug product” as used herein is the finished dosage form of the product.

The term “detectable presence” of a microorganism refers to a confirmed positive detection in a sample of a microorganism genus, species and/or strain by any method known in the art. Confirmation may be a positive test interpretation by a skilled practitioner and/or by repeating the method.

The term “microbiome” or “microbiomic” or “microbiota” as used herein refers to microbiological ecosystems. These ecosystems are a community of commensal, symbiotic and pathogenic microorganisms found in and on all animals and plants.

The term “microorganism” as used herein refers to an organism that can be seen only with the aid of a microscope and that typically consists of only a single cell. Microorganisms include bacteria, protozoans and fungi.

The term “niche” and “niche conditions” as used herein refers to the ecologic array of environmental and nutritional requirements that are required for a particular species of microorganism. The definitions of the values for the niche of a species defines the places in the particular biomes that can be physically occupied by that species and defines the possible microbial competitors.

The term “colonization” as used herein refers to the persistent detectable presence of a microorganism on a body surface, e.g., a dermal or mucosal surface, without causing disease in the individual.

The term “co-colonization” as used herein refers to simultaneous colonization of a niche in a site on a subject by two or more strains, or variants within the same species of microorganisms. For example, the term “co-colonization” may refer to two or more strains or variants simultaneously and non-transiently occupying the same niche. The term non-transiently refers to positive identification of a strain or variant at a site in a subject over time at two or more time subsequent points in a multiplicity of samples obtained from the subject at least two weeks apart.

The term “target microorganism” as used herein refers to a wild-type microorganism or a parent synthetic microorganism, for example, selected for molecular modification to provide a synthetic microorganism. The target microorganism may be of the same genus and species as the undesirable microorganism, which may cause a pathogenic infection.

The “target microorganism” may be selected from the group consisting of Staphylococcus aureus, coagulase-negative staphylococci (CNS), Streptococci Group A, Streptococci Group B, Streptococci Group C, Streptococci Group C & G, Staphylococcus spp., Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, and Pseudomonas aeruginosa.

The “target strain” may be the particular strain of target microorganism selected for molecular modification to provide the synthetic microorganism. Preferably, the target strain is sensitive to one or more antimicrobial agents. For example, if the undesirable microorganism is a Methicillin resistant Staphylococcus aureus (MRSA) strain, the target microorganism may be an antibiotic susceptible target strain, or Methicillin Susceptible Staphylococcus aureus (MSSA) strain, such as WT-502a. In some embodiments, the target microorganism may be of the same species as the undesirable microorganism. In some embodiments, the target microorganism may be a different strain, but of the same species as the undesirable microorganism.

The term “bacterial replacement” or “non-co-colonization” as used herein refers to the principle that only one variant/strain of one species can occupy any given niche within the biome at any given time.

The term “action gene” as used herein refers to a preselected gene to be incorporated to a molecular modification, for example, in a target microorganism. The molecular modification comprises the action gene operatively associated with a regulatory region comprising an inducible promoter. The action gene may include exogenous DNA. The action gene may include endogenous DNA. The action gene may include DNA having the same or substantially identical nucleic acid sequence as an endogenous gene in the target microorganism. The action gene may encode a molecule, such as a protein, that when expressed in an effective amount causes an action or phenotypic response within the cell. The action or phenotypic response may be selected from the group consisting of cell suicide (kill switch molecular modification comprising a cell death gene), prevention of horizontal gene transfer (virulence block molecular modification), metabolic modification (metabolic molecular modification), reporter gene, and production of a desirable molecule (nano factory molecular modification).

The term “kill switch” or “KS” as used herein refers to an intentional molecular modification of a synthetic microorganism, the molecular modification comprising a cell death gene operably linked to a regulatory region comprising an inducible promoter, genetic element or cassette, wherein induced expression of the cell death gene in the kill switch causes cell death, arrest of growth, or inability to replicate, of the microorganism in response to a specific state change such as a change in environmental condition of the microorganism.

For example, in the synthetic microorganism comprising a kill switch, the inducible first promoter may be activated by the presence of blood, serum, plasma, and/or heme, wherein the upregulation and transcription/expression of the operably associated cell death gene results in cell death of the microorganism or arrested growth of the microorganism so as to improve the safety of the synthetic microorganism.

The target microorganism may be, for example, a Staphylococcus species, Escherichia species, or a Streptococcus species.

The target microorganism may be a Staphylococcus species or an Escherichia species. The target microorganism may be a Staphylococcus aureus target strain. The action gene may be a toxin gene. Toxin genes may be selected from sprA1, sma1, rsaE, relF, 187/lysK, Holin, lysostaphin, SprG1, sprG2, sprG3, SprA2, mazF, Yoeb-sa2. The inducible promoter gene may be a serum, blood, plasma, heme, CSF, interstitial fluid, or synovial fluid inducible promoter gene, for example, selected from isdB, leuA, hlgA, hlgA2, isdG, sbnC, sbnE, hlgB, SAUSA300_2616, splF, fhuB, hlb, hrtAB, IsdG, LrgA, SAUSA300_2268, SAUSA200_2617, SbnE, IsdI, LrgB, SbnC, HlgB, IsdG, SplF, IsdI, LrgA, HlgA2, CH52_04385, CH52_05105, CH52_06885, CH52_10455, PsbnA, or sbnA.

The target microorganism may be a Streptococcus species. The target microorganism may be a Streptococcus agalactiae, Streptococcus pneumonia, or Streptococcus mutans target strain. The action gene may be a toxin gene. The toxin gene may be selected from a RelE/ParE family toxin, ImmA/IrrE family toxin, mazEF, ccd or relBE, Bro, abiGII, HicA, COG2856, RelE, or Fic. The inducible promoter gene may be a serum, blood, plasma, heme, CSF, interstitial fluid, or synovial fluid inducible promoter gene, for example, selected from a Regulatory protein CpsA, Capsular polysaccharide synthesis protein CpsH, Polysaccharide biosynthesis protein CpsL, R3H domain-containing protein, Tyrosine-protein kinase CpsD, Capsular polysaccharide biosynthesis protein CpsC, UDP-N-acetylglucosamine-2-epimerase NeuC, GTP pyrophosphokinase RelA, PTS system transporter subunit IIA, Glycosyl transferase CpsE, Capsular polysaccharide biosynthesis protein CpsJ, NeuD protein, IgA-binding β antigen, Polysaccharide biosynthesis protein CpsG, Polysaccharide biosynthesis protein CpsF, or a Fibrinogen binding surface protein C FbsC.

The term “exogenous DNA” as used herein refers to DNA originating outside the target microorganism. The exogenous DNA may be introduced to the genome of the target microorganism using methods described herein. The exogenous DNA may or may not have the same or substantially identical nucleic acid sequence as found in a target microorganism, but may be inserted to a non-natural location in the genome. For example, exogenous DNA may be copied from a different part of the same genome it is being inserted into, since the insertion fragment was created outside the target organism (i.e. PCR, synthetic DNA, etc.) and then transformed into the target organism, it is exogenous.

The term “exogenous gene” as used herein refers to a gene originating outside the target microorganism. The exogenous gene may or may not have the same or substantially identical nucleic acid sequence as found in a target microorganism, but may be inserted to a non-natural location in the genome. Transgenes are exogenous DNA sequences introduced into the genome of a microorganism. These transgenes may include genes from the same microorganism or novel genes from a completely different microorganism. The resulting microorganism is said to be transformed.

The term “endogenous DNA” as used herein refers to DNA originating within the genome of a target microorganism prior to genomic modification.

The term “endogenous gene” as used herein refers to a gene originating within the genome of a target microorganism prior to genomic modification.

As used herein the term “minimal genomic modification” (MGM) refers to a molecular modification made to a target microorganism, wherein the MGM comprises an action gene operatively associated with a regulatory region comprising an inducible promoter gene, wherein the action gene and the inducible promoter are not operably associated in the unmodified target microorganism. Either the action gene or the inducible promoter gene may be exogenous to the target microorganism.

For example, a synthetic microorganism having a first minimal genomic modification may contain a first recombinant nucleic acid sequence consisting of a first exogenous control arm and a first exogenous action gene, wherein the first exogenous action gene is operatively associated with an endogenous regulatory region comprising an endogenous inducible promoter gene.

Inserting an action gene into an operon in the genome will tie the regulation of that gene to the native regulation of the operon into which it was inserted. It is possible to further regulate the transcription or translation of the inserted action gene by adding additional DNA bases to the sequence being inserted into the genome either upstream, downstream, or inside the reading frame of the action gene.

As used herein the term “control arm” refers to additional DNA bases inserted either upstream and/or downstream of the action gene in order to help to control the transcription of the action gene or expression of a protein encoded thereby. The control arm may be located on the terminal regions of the inserted DNA. Synthetic or naturally occurring regulatory elements such as micro RNAs (miRNA), antisense RNA, or proteins can be used to target regions of the control arms to add an additional layer of regulation to the inserted gene.

When the ratio of the regulatory elements to action genes are in sufficient excess, leaky expression of the action gene may be suppressed. When the expression of the operon containing the action gene is induced and/or the expression of the regulatory elements are suppressed, the concentration of action gene mRNA overwhelms the regulatory elements allowing full transcription and translation of the action gene or genes.

For example, a control arm may be employed in a kill switch molecular modification comprising an sprA1 gene, where the control arm may be inserted to the 5′ untranslated region (UTR) in front of the sprA1 gene. When the sprA1 gene from BP_001 was PCR amplified the native sequence just upstream of that (i.e. control arm) was included. The sprA1(AS) binds to the sprA1 mRNA in two places, once right after the start codon, and once in the 5′ UTR blocking the RBS. In order to get maximum efficiency from the sprA1(AS) to suppress the translation of the PepA1 protein, the control arm sequence was retained.

As further examples, the control arm for the kill switch molecular modification comprising an sprA2 gene may also include a 5′ UTR where its antisense binds, and the control arm for the sprG1 gene may include a 3′ UTR where its antisense antitoxin binds, so the control arm is not just limited to regions upstream of the start codon. In some embodiments, the start codon for the action gene may be inserted very close to the stop codon for gene in front of it, or within a few bases behind the previous gene's stop codon and an RBS and then the action gene. In some embodiments, where the molecular modification is a kill switch molecular modification, and the action gene is sprA1, the control arm may be a sprA1 5′ UTR sequence to give better regulation of the action gene with minimal impact on the promoter gene, for example, isdB.

The control arm sequence may be employed as another target to “tune” the expression of the action gene. By making base pair changes, the binding efficiency of the antisense may be used to tweak the level of regulation.

For example, the antitoxin for the sprA1 toxin gene is an antisense sprA1 RNA (sprA1_{AS}) and regulates the translation of the sprA1 toxin (PepA1). When the concentration of sprA1_{AS} RNA is at least 35 times greater than the sprA1 mRNA, PepA1 is not translated and the cell is able to function normally. When the ratio of sprA1_{AS}:sprA1 gets below about 35:1, suppression of sprA1 translation is not complete and the cell struggles to grow normally. At a certain point the ratio of sprA1_{AS}:sprA1 RNA is low enough to allow enough PepA1 translation to induce apoptosis and kill the cells.

The term “cell death gene” or “toxin gene” refers to a gene that when induced causes a cell to enter a state where it either ceases reproduction, alters regulatory mechanisms of the cell sufficiently to permanently disrupt cell viability, induces senescence, or induces fatal changes in the genetic or proteomic systems of the cell. For example, the cell death gene may be a toxin gene encoding a toxin protein or toxin peptide. The toxin gene may be selected from the group consisting of sprA1, sma1, rsaE, relF, 187/lysK, holin, lysostaphin, sprG1, sprA2, sprG2, sprG3, mazF, and yoeb-sa2. The toxin gene may be sprA1. In one embodiment, the toxin gene may encode a toxin protein or toxin peptide. In some embodiments, the toxin protein or toxin peptide may be bactericidal to the synthetic microorganism. In some embodiments, the toxin protein or toxin peptide may be bacteriostatic to the synthetic microorganism.

The term “antitoxin gene” refers to a gene encoding an antitoxin RNA antisense molecule or an antitoxin protein or another antitoxin molecule specific for a cell death gene or a product encoded thereby

The term “virulence block” or “V-block” refers to a molecular modification of a microorganism that results in the organism have decreased ability to accept foreign DNA from other strains or species effectively resulting in the organism having decreased ability to acquire exogenous virulence or antibiotic resistance genes.

The term “nanofactory” as used herein refers to the molecular modification of a microorganism that results in the production of a product—either primary protein, polypeptide, amino acid or nucleic acid or secondary products of these modifications to beneficial effect.

The term “toxin protein” or “toxin peptide” as used herein refers to a substance produced internally within a synthetic microorganism in an effective amount to cause deleterious effects to the microorganism without causing deleterious effects to the subject that it colonizes.

The term “molecular modification” or “molecularly engineered” as used herein refers to an intentional modification of the genes of a microorganism using any gene editing method known in the art, including but not limited to recombinant DNA techniques as described herein below, NgAgo, mini-Cas9, CRISPR-Cpf1, CRISPR-C2c2, Target-AID, Lambda Red, Integrases, Recombinases, or use of phage techniques known in the art. The DNA may be sequenced and manipulated chemically or by using molecular biology techniques, for example, to arrange one or more elements, e.g., regulatory regions, promoters, toxin genes, antitoxin genes, or other domains into a suitable configuration, or to introduce codons, delete codons, optimize codons, create cysteine residues, modify, add or delete amino acids, etc. Molecular modification may include, for example, use of plasmids, gene insertion, gene knock-out to excise or remove an undesirable gene, frameshift by adding or subtracting base pairs to break the coding frame, exogenous silencing, e.g., by using inducible promoter or constitutive promoter which may be embedded in DNA encoding, e.g. RNA antisense antitoxin, production of CRISPR-cas9 or other editing proteins to digest, e.g., incoming virulence genes using guide RNA, e.g., linked to an inducible promoter or a constitutive promoter, or a restriction modification/methylation system, e.g., to recognize and destroy incoming virulence genes to increase resistance to horizontal gene transfer. The molecular modification (e.g. kill switch, expression clamp, and/or v-block) may be durably incorporated to the synthetic microorganism by inserting the modification into the genome of the synthetic microorganism.

The synthetic microorganism may further comprise additional molecular modifications, (e.g., a nanofactory), which may be incorporated directly into the bacterial genome, or into plasmids, in order to tailor the duration of the effect of, e.g., the nanofactory production, and could range from short term (with non-replicating plasmids for the bacterial species) to medium term (with replicating plasmids without addiction dependency) to long term (with direct bacterial genomic manipulation).

The molecular modifications may confer a non-native attribute desired to be durably incorporated into the host microbiome, may provide enhanced safety or functionality to organisms in the microbiome or to the host microbiome overall, may provide enhanced safety characteristics, including kill switch(s) or other control functions. In some embodiments the safety attributes so embedded may be responsive to changes in state or condition of the microorganism or the host microbiome overall.

The molecular modification may be incorporated to the synthetic microorganism in one or more, two or more, five or more, 10 or more, 30 or more, or 100 or more copies, or no more than one, no more than three, no more than five, no more than 10, no more than 30, or in no more than 100 copies.

The term “genomic stability” or “genomically stable” as used herein in reference to the synthetic microorganism means the molecular modification is stable over at least 500 generations of the synthetic microorganism as assessed by any known nucleic acid sequence analysis technique.

The term “functional stability” or “functionally stable” as used herein in reference to the synthetic microorganism means the phenotypic property imparted by the action gene is stable over at least 500 generations of the synthetic microorganism.

For example, a functionally stable synthetic microorganism comprising a kill switch molecular modification will exhibit cell death within at least about 2 hours, 4 hours, or 6 hours after exposure to blood, serum, or plasma over at least 500 generations of the synthetic microorganism as assessed by any known in vitro culture technique. Functional stability may be assessed, for example, after at least about 500 generations by comparative growth of the synthetic microorganism in a media with or without presence of a change in state. For example, a synthetic microorganism comprising a cell death gene may exhibit cell death following exposure to blood, serum or plasma, for example by comparing cfu/mL over at least about 2 hours, at least about 4 hours, or at least about 6 hours, wherein a decrease in cfu/mL of at least about 3 orders of magnitude, or at least about 4 orders of magnitude compared to starting cfu/mL at t=0 hrs is exhibited. Functional stability of a synthetic microorganism may also be assessed in an in vivo model. For example, a mouse tail vein inoculation bacteremia model may be employed. Mice administered a synthetic microorganism (10\hat{ }7 CFU/mL) having a KS molecular modification, such as a synthetic Staph aureus having a KS molecular modification will exhibit survival over at least about 4 days, 5 days, 6 days, or 7 days, compared to mice administered the same dose of WT Staph aureus exhibiting death or moribund condition over the same time period.

The term “recurrence” as used herein refers to re-colonization of the same niche by a decolonized microorganism.

The term “pharmaceutically acceptable” refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a carrier that is physiologically acceptable to the treated subject while retaining the integrity and desired properties of the synthetic microorganism with which it is administered. Exemplary pharmaceutically acceptable carriers include physiological saline or phosphate-buffered saline. Sterile Luria broth, tryptone broth, or TSB may be also employed as carriers. Other physiologically acceptable carriers and their formulations are provided herein or are known to one skilled in the art and described, for example, in Remington's Pharmaceutical Sciences, (20th edition), ed. A. Gennaro, 2000, Lippincott, Williams & Wilkins, Philadelphia, Pa.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described.

Vectors and Target Microorganisms

Also described herein are vectors comprising polynucleotide molecules, as well as target cells transformed with such vectors. Polynucleotide molecules described herein may be joined to a vector, which include a selectable marker and origin of replication, for the propagation host of interest. Target cells are genetically engineered to include these vectors and thereby transcribe RNA and express polypeptides. Vectors herein include polynucleotides molecules operably linked to suitable transcriptional or translational regulatory sequences, such as those for microbial target cells. Examples of regulatory sequences include transcriptional promoters, operators, or enhancers, mRNA ribosomal binding sites, and appropriate sequences which control transcription and translation. Nucleotide sequences as described herein are operably linked when the regulatory sequences herein functionally relate to, e.g., a cell death gene encoding polynucleotide.

Typical vehicles include plasmids, shuttle vectors, baculovirus, inactivated adenovirus, and the like. In certain examples described herein, the vehicle may be a modified pIMAY, pIMAYz, or pKOR integrative plasmid, as discussed herein.

A target microorganism may be selected from any microorganism having the ability to durably replace a specific undesirable microorganism after decolonization. The target microorganism may be a wild-type microorganism that is subsequently engineered to enhance safety by methods described herein. The target microorganism may be selected from a bacterial, fungal, or protozoal target microorganism. The target microorganism may be a strain capable of colonizing a dermal and/or mucosal niche in a subject. The target microorganism may be a wild-type microorganism, or a synthetic microorganism that may be subjected to further molecular modification. The target microorganism may be selected from a genus selected from the group consisting of Staphylococcus, Acinetobacter, Corynebacterium, Streptococcus, Escherichia, Mycobacterium, Enterococcus, Bacillus, Klebsiella, and Pseudomonas. The target microorganism may be selected from the group consisting of Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa. The target microorganism may be a species having a genus selected from the group consisting of Candida or Cryptococcus. The target microorganism may be Candida parapsilosis, Candida krusei, Candida tropicalis, Candida albicans, Candida glabrata, or Cryptococcus neoformans.

The target microorganism may be of the same genus and species as the undesirable microorganism, but of a different strain. For example, the undesirable microorganism may be an antibiotic-resistant Staphylococcus aureus strain, such as an MRSA strain. The antibiotic-resistant Staphylococcus aureus stain may be a pathogenic strain, which may be known to be involved in dermal infection, mucosal infection, bacteremia, and/or endocarditis. Where the undesirable microorganism is a Staphylococcus aureus strain, e.g., an MRSA, the target microorganism may be, e.g., a less pathogenic strain which may be an isolated strain such as Staphylococcus aureus target cell such as an RN4220 or 502a strain, and the like. Alternatively, the target cell may be of the same strain as the undesirable microorganism. In another example, the undesirable microorganism is an Escherichia coli strain, for example, a uropathogenic E. coli type 1 strain or p-fimbriated strain, for example, a strain involved in urinary tract infection, bacteremia, and/or endocarditis. In another example, the undesirable strain is a Cutibacterium acnes strain, for example a strain involved in acnes vulgaris, bacteremia, and/or endocarditis. In another example, the undesirable microorganism is a Streptococcus mutans strain, for example, a strain involved in S. mutans endocarditis, dental caries.

Model Antibiotic-Susceptible Target Microorganism

The target microorganism may be an antibiotic-susceptible microorganism of the same species as the undesirable microorganism. In one embodiment, the undesirable microorganism is an MRSA strain and the replacement target microorganism is an antibiotic susceptible Staphylococcus aureus strain. The antibiotic susceptible microorganism may be Staphylococcus aureus strain 502a (“502a”). 502a is a coagulase positive, penicillin sensitive, nonpenicillinase producing staphylococcus, usually lysed by phages 7, 47, 53, 54, and 77. Serologic type (b)ci. Unusual disc antibiotic sensitivity pattern is exhibited by 502a because this strain is susceptible to low concentrations of most antibiotics except tetracycline; resistant to 5 g, but sensitive to 10 μg of tetracycline. In some embodiments, the 502a strain may be purchased commercially as Staphylococcus aureus subsp. Aureus Rosenbach ATCC®27217™.

Unfortunately, even an antimicrobial agent-susceptible target microorganism may cause systemic infection. Therefore, as provided herein, the target microorganism is subjected to molecular modification to incorporate regulatory sequences including, e.g., an inducible first promoter for expression of the cell death gene, v-block, or nanofactory, in order to enhance safety and reduce the likelihood of pathogenic infection as described herein.

The target microorganism and/or the synthetic microorganism comprises (i) the ability to durably colonize a niche in a subject following decolonization of the undesirable microorganism and administering the target or synthetic microorganism to a subject, and (ii) the ability to prevent recurrence of the undesirable microorganism in the subject for a period of at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

Selection of a Target Microorganism for MRSA

Selection of the target microorganism may be performed by decolonizing the target microorganism and replacing with a putative target microorganism, as described herein. For example, the undesirable microorganism Methicillin-Resistant Staphylococcus aureus (MRSA) is the cause of a disproportionate amount of invasive bacterial infections worldwide. The colonization state for Staphylococcus aureus is regarded as a required precondition for most invasive infections. However, decolonization with standard antiseptic regimens has been studied as a method for reducing MRSA colonization and infections with mixed results. In one example provided herein, the feasibility and durability of a novel decolonization approach to undesirable microorganism MRSA by using intentional recolonization with a different Staphylococcus aureus strain as a candidate target microorganism was performed in hopes of improving duration of effect versus standard decolonization. Example 1 discloses the study in which a total of 765 healthy volunteers were screened for Staphylococcus aureus colonization. The overall MRSA rate for the screened population was 8.5%. A cohort of 53 MRSA colonized individuals participated in a controlled study of a decolonization/recolonization therapy using Staphylococcus aureus 502a WT strain BioPlx-01 vs. a control group of standard decolonization alone. Duration of MRSA absence from the colonization state as well as persistence of the intentional MSSA recolonization was monitored for 6 months. The control group (n=15) for the efficacy portion of the MRSA decolonization protocol showed MRSA recurrence of 60% at the 4 week time point. The test group employing the BioPlx-01WT protocol (n=34) showed 0% MRSA recurrence at the 8 week primary endpoint and continued to show no evidence of MRSA recurrence out to 26 weeks. Instead these participants exhibited surprising persistence of colonization with MSSA likely indicating ongoing colonization with the Staphylococcus aureus 502a BioPlx-01WT strain product out to 26 weeks. In addition, the components of the BioPlx-01WT in a phosphate buffered saline composition used in the decolonization/recolonization therapy showed no evidence of dermal irritation in a separate cohort of 55 participants. Therefore, target strain Staphylococcus aureus 502a BioPlx-01WT decolonization/recolonization protocol provides longer durability of decolonization from MRSA strains than standard decolonization and shows no observed negative dermal effects.

Methods for Determining Detectable Presence of a Microorganism

Any method known in the art may be employed for determination of the detectable presence of a microorganism genus, species and strain. An overview of methods may be found in Aguilera-Arreola MG. Identification and Typing Methods for the Study of Bacterial Infections: a Brief Review and Mycobacterial as Case of Study. Arch Clin Microbiol. 2015, 7:1, which is incorporated herein by reference.

The detectable presence of a genus, species and/or strain of a bacteria may be determined by phenotypic methods and/or genotypic methods. Phenotypic methods may include biochemical reactions, serological reactions, susceptibility to anti-microbial agents, susceptibility to phages, susceptibility to bacteriocins, and/or profile of cell proteins. One example of a biochemical reaction is the detection of extracellular enzymes. For example, staphylococci produce many different extracellular enzymes including DNAase, proteinase and lipases. Gould, Simon et al., 2009, The evaluation of novel chromogenic substrates fro detection of lipolytic activity in clinical isolates of Staphylococcus aureus and MRSA from two European study groups. FEMS Microbiol Let 297; 10-16. Chomogenic substrates may be employed for detection of extracellular enzymes. For example, CHROMager™ MRSA chromogenic media (CHROMagar, Paris, France) may be employed for isolation and differentiation of Methicillin Resistant Staphylococcus aureus (MRSA) including low level MRSA. Samples are obtained from, e.g., nasal, perineal, throat, rectal specimens are obtained with a possible enrichment step. If the agar plate has been refrigerated, it is allowed to warm to room temperature before inoculation. The sample is streaked onto plate followed by incubation in aerobic conditions at 37° C. for 18-24 hours. The appearance of the colonies is read, wherein MRSA colonies appear as rose to mauve colored, Methicillin Susceptible Staphylococcus aureus (MSSA) colonies are inhibited, and other bacteria appear as blue, colorless or inhibited colonies. Definite identification as MRSA requires, in addition, a final identification as Staphylococcus aureus. For example, CHROMagar™ Staph aureus chromogenic media may be employed where S. aureus appears as mauve, S. saprophyticus appears turquoise blue, E. coli, C. albicans and E. faecalis are inhibited. For detection of Group B Streptococcus(GBS) (S. agalactiae), CHROMagar™ StrepB plates may be employed, wherein Streptococcus agalactiae (group B) appear mauve, Enterococcus spp. and E. faecalis appear steel blue, Lactobacilli, leuconostoc and lactococci appear light pink, and other microorganisms are blue, colorless or inhibits. For detection of various Candida spp., CHROMager™ Candida chromogenic media may be employed. Candida species are involved in superficial oropharyngeal and urogenital infections. Although C. albicans remains a major species involved, other types such as C. tropicalis, C. krusai, or C. glabrata have increased as new antifungal agents have worked effectively against C. albicans. Sampling and direct streaking of skin, sputum, urine, vaginal specimens samples and direct streaking or spreading onto plate, followed by incubation in aerobic conditions at 30-37° C. for 48 hours, and reading of plates for colony appearance where C. albicans is green, C. tropicalis is metallic blue, C. krusei is pink and fuzzy, C. kefyr and C. glabrata are mauve-brown, and other species are white to mauve.

Genotypic methods for genus and species identification may include hybridization, plasmids profile, analysis of plasmid polymorphism, restriction enzymes digest, reaction and separation by Pulsed-Field Gel Electrophoresis (PFGE), ribotyping, polymerase chain reaction (PCR) and its variants, Ligase Chain Reaction (LCR), Transcription-based Amplification System (TAS), or any of the methods described herein.

Identification of a microbe can be performed, for example, by employing Galileo™ Antimicrobial Resistance (AMR) detection software (Arc Bio LLC, Menlo Park, Calif. and Cambridge, Mass.) that provides annotations for gram-negative bacterial DNA sequences.

The microbial typing method may be selected from genotypic methods including Multilocus Sequence Typing (MLST) which relies on PCR amplification of several housekeeping genes to create allele profiles; PCR-Extragenic Palindromic Repetitive Elements (rep-PCR) which involves PCR amplification of repeated sequences in the genome and comparison of banding patterns; AP-PCR which is Polymerase Chain Reaction using Arbitrary Primers; Amplified Fragment Length Polymorphism (AFLP) which involves enzyme restriction digestion of genomic DNA, binding of restriction fragments and selective amplification; Polymorphism of DNA Restriction Fragments (RFLP) which involves Genomic DNA digestion or of an amplicon with restriction enzymes producing short restriction fragments; Random Amplified Polymorphic DNA (RAPD) which employs marker DNA fragments from PCR amplification of random segments of genomic DNA with single primer of arbitrary nucleotide sequence; Multilocus Tandem Repeat Sequence Analysis (MLVA) which involves PCR amplification of loci VTR, visualizing the polymorphism to create an allele profile; or Pulsed-Fields Gel Electrophoresis (PFGE) which involves comparison of macro-restriction fragments. PFGE method of electrophoresis is capable of separating fragments of a length higher than 50 kb up to 10 Mb, which is not possible with conventional electrophoresis, which can separate only fragments of 100 bp to 50 kb. This capacity of PFGE is due to its multidirectional feature, changing continuously the direction of the electrical field, thus, permitting the re-orientation of the direction of the DNA molecules, so that these can migrate through the agarose gel, in addition to this event, the applied electrical pulses are of different duration, fostering the reorientation of the molecules and the separation of the fragments of different size. One PFGE apparatus may be the Contour Clamped Homogeneous Electric Fields (CHEF, BioRad). Pulsed-filed gel electrophoresis (PFGE) is considered a gold standard technique for MRSA typing, because of its high discriminatory power, but its procedure is complicated and time consuming. The spa gene encodes a cell wall component of Staphylococcus aureus protein A, and exhibits polymorphism. The sequence based-spa typing can be used as a rapid test screen. Narukawa et al 2009 Tohoku J Exp Med 2009, 218, 207-213.

Methods and compositions are provided herein for suppressing (decolonizing) and replacing an undesirable microorganism with a new synthetic microorganism in order to durably displace and replace the undesirable microorganism from the microbiological ecosystem with a new microorganism so as to prevent the recurrence of the original undesirable organism (referred to here as niche or ecological interference).

In some embodiments, methods are provided to prevent colonization, prevent infection, decrease recurrence of colonization, or decrease recurrence of a pathogenic infection of a undesirable microorganism in a subject, comprising decolonization and administering a synthetic strain comprising a molecular modification that decreases the ability of the synthetic microorganism to cause disease to the subject relative to the wild type target strain where the microorganism is selected from the group consisting of Acinetobacter johnsonii, Acinetobacter baumannii, Staphylococcus aureus, Staphylococcus epidermidis, Staphylococcus lugdunensis, Staphylococcus warneri, Staphylococcus saprophyticus, Corynebacterium acnes, Corynebacterium striatum, Corynebacterium diphtheriae, Corynebacterium minutissimum, Cutibacterium acnes, Propionibacterium acnes, Propionibacterium granulosum, Streptococcus pyogenes, Streptococcus aureus, Streptococcus agalactiae, Streptococcus mitis, Streptococcus viridans, Streptococcus pneumoniae, Streptococcus anginosus, Streptococcus constellatus, Streptococcal intermedius, Streptococcus agalactiae, Pseudomonas aeruginosa, Pseudomonas oryzihabitans, Pseudomonas stutzeri, Pseudomonas putida, and Pseudomonas fluorescens.

In some embodiments, a method is provided to prevent transmission by a subject, or recurrence of colonization or infection, of a pathogenic microorganism in a subject, comprising suppressing the pathogenic microorganism in the subject, and replacing the pathogenic microorganism by topically administering to the subject a composition comprising a benign microorganism of the same species, different strain. The method may further comprise promoting the colonization of the benign microorganism. In some embodiments, the benign microorganism is a synthetic microorganism having at least one molecular modification comprising a first cell death gene operably linked to a first regulatory region comprising a first promoter, wherein the first promoter is activated in the presence of human serum or blood. In some embodiments, the first promoter is not activated during colonization of dermal or mucous membranes in a human subject.

In some embodiments, method is provided to prevent transmission by a subject, or recurrence of colonization or infection, of a methicillin-resistant Staphylococcus aureus (MRSA) in a subject, comprising suppressing the MRSA in the subject, and replacing the MRSA by topically administering to the subject a methicillin susceptible Staphylococcus aureus (MSSA) of the same species, different strain. The method may further comprise promoting the colonization of the MSSA in the subject.

A method is provided to prevent transmission by a subject, or recurrence of colonization or infection, of a undesirable microorganism in a subject, comprising suppressing the undesirable microorganism in the subject, and replacing the undesirable microorganism by administering to the subject a second microorganism of the same species, different strain. The method may further comprise promoting the colonization of the second microorganism. In some embodiments, the undesirable microorganism is a drug-resistant pathogenic microorganism. In some embodiments, the second microorganism is a drug-susceptible microorganism. In some embodiments, the second microorganism is a synthetic microorganism.

Suppression/Decolonization

An undesirable microorganism may be suppressed, or decolonized, by topically applying a disinfectant, antiseptic, or biocidal composition directly to the skin or mucosa of the subject, for example, by spraying, dipping, or coating the affected area, optionally the affected area and adjacent areas, or greater than 25%, 50%, 75%, or greater than 90% of the external or mucosal surface area of the subject with the disinfectant, antiseptic, or biocidal composition. In some embodiments, the affected area, or additional surface areas are allowed to air dry or are dried with an air dryer under gentle heat, or are exposed to ultraviolet radiation or sunlight prior to clothing or dressing the subject. In one embodiment, the suppression comprises exposing the affected area, and optionally one or more adjacent or distal areas of the subject, with ultraviolet radiation. In various embodiments, any commonly employed disinfectant, antiseptic, or biocidal composition may be employed. In one embodiment, a disinfectant comprising chlorhexidine or a pharmaceutically acceptable salt thereof is employed.

In some embodiments, the bacteriocide, antiseptic, astringent, and/or antibacterial agent is selected from the group consisting of alcohols (ethyl alcohol, isopropyl alcohol), aldehydes (glutaraldehyde, formaldehyde, formaldehyde-releasing agents (noxythiolin=oxymethylenethiourea, tauroline, hexamine, dantoin), o-phthalaldehyde), anilides (triclocarban=TCC=3,4,4′-trichlorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), quaternary ammonium compounds (cetrimide, benzalkonium chloride, cetyl pyridinium chloride), silver compounds (silver sulfadiazine, silver nitrate), peroxy compounds (hydrogen peroxide, peracetic acid), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), botanical extracts (Melaleuca spp. (tea tree oil), Cassia fistula Linn, Baekea frutescens L., Melia azedarach L., Muntingia calabura, Vitis vinifera L, Terminalia avicennioides Guill & Perr., Phylantus discoideus muel. Muel-Arg., Ocimum gratissimum Linn., Acalypha wilkesiana Muell-Arg., Hypericum pruinatum Boiss.&Bal., Hypericum olimpicum L. and Hypericum sabrum L., Hamamelis virginiana (witch hazel), Eucalyptus spp., Rosmarinus officinalis spp. (rosemary), Thymus spp. (thyme), Lippia spp. (oregano), Cymbopogon spp. (lemongrass), Cinnamomum spp., Geranium spp., Lavendula spp.), and topical antibiotic compounds (bacteriocins; mupirocin, bacitracin, neomycin, polymyxin B, gentamicin).

Suppression of the undesirable microorganism also may be performed by using photosensitizers instead of or in addition to, e.g., topical antibiotics. For example, Peng Zhang et al., Using Photosensitizers Instead of Antibiotics to Kill MRSA, GEN News Highlights, Aug. 20, 2018; 48373, developed a technique using light to activate oxygen, which suppresses to microbial growth. Photosensitizers, such as dye molecules, become excited when illuminated with light. The photosensitizers convert oxygen into reactive oxygen species that kill the microbes, such as MRSA. In order to concentrate the photosensitizers to improve efficacy, water-dispersible, hybrid photosensitizers were developed by Zhang et al., comprising noble metal nanoparticles decorated with amphiphilic polymers to entrap molecular photosensitizers. The hybrid photosensitizers may be applied to a subject, for example, on a dermal surface or wound, in the form of a spray, lotion or cream, then illuminated with red or blue light to reduce microbial growth.

A decolonizing composition may be in the form of a topical solution, lotion, or ointment form comprising a disinfectant, biocide photosensitizer or antiseptic compound and one or more pharmaceutically acceptable carriers or excipients. In one specific example, an aerosol disinfectant spray is employed comprising chlorhexidine gluconate (0.4%), glycerin (10%), in a pharmaceutically acceptable carrier, optionally containing a dye to mark coverage of the spray. In one embodiment, the suppressing step comprises administration to one or more affected areas, and optionally one or more surrounding areas, with a spray disinfectant as disclosed in U.S. Pat. Nos. 4,548,807 and/or 4,716,032, each of which is incorporated herein by reference in its entirety. The disinfectant spray may be commercially available, for example, Fight Bac®, Deep Valley Farm, Inc., Brooklyn, Conn. Other disinfectant materials may include chlorhexidine or salts thereof, such as chlorhexidine gluconate, chlorhexidine acetate, and other diguanides, ethanol, SD alcohol, isopropyl alcohol, p-chloro-o-benzylphenol, o-phenylphenol, quaternary ammonium compounds, such as n-alkyl/dimethyl ethyl benzyl ammonium chloride/n-alkyl dimethyl benzyl ammonium chloride, benzalkonium chloride, cetrimide, methylbenzethonium chloride, benzethonium chloride, cetalkonium chloride, cetylpyridinium chloride, dofanium chloride, domiphen bromide, peroxides and permanganates such as hydrogen peroxide solution, potassium permanganate solution, benzoyl peroxide, antibacterial dyes such as proflavine hemisulphate, triphenylmethane, Brilliant green, Crystal violet, Gentian violet, quinolone derivatives such as hydroxyquinoline sulphate, potassium hydroxyquinoline sulphate, chloroquinaldol, dequalinium chloride, di-iodohydroxyquinoline, Burow's solution (aqueous solution of aluminum acetate), bleach solution, iodine solution, bromide solution. Various Generally Recognized As Safe (GRAS) materials may be employed in the disinfectant or biocidal composition including glycerin, and glycerides, for example but not limited to mono- and diglycerides of edible fat-forming fatty acids, diacetyl tartaric acid esters of mono- and diglycerides, triacetin, acettooleins, acetostearins, glyceryl lactopalmitate, glyceryl lactooleate, and oxystearins.

Decolonizing agents may include a teat disinfectant, for example, as a barrier teat dip, spray, foam, or powder. The barrier teat dip, spray, foam or powder may be selected from an iodine-based dip (e.g. Tri-Fender™, DeLaval; Blockade®, DeLaval; Iodozyme™, DeLaval; Bovidine®, DeLaval; DelaBarrier®, DeLaval; WestAgro West Dip™, Della Soft™, Della One Plus™, Triumph™, Quarter Mate® Plus, DeLaval; Sprayable Udderdine™ 110 Barrier, BouMatic; Udderdine™ Apex, BouMatic, Apex™ 5000, BouMatic), lactic acid teat dip (e.g., LactiFence™, DeLaval; Lactisan™, DeLaval; Lactisan™ (Winter, DeLaval), Chlorine dioxide (e.g., Vanquish™, DeLaval; Gladiator™, BouMatic; Gladiator BLU Barrier, Boumatic), hydrogen peroxide (e.g., Prima™, DeLaval), glycolic acid (e.g., OceanBlu™, DeLaval); chlorhexidine (e.g., Sani-Cling™, Boumatic), chlorhexidine gluconate (e.g., Fight Bac(TN), Deep Valley Farm, Inc.), sodium hypochlorite, iodophor, chlorine, acidified sodium chlorite (e.g., with lactic acid or mandelic acid), dodecylbenzenesulfonic acid, C6-C14 fatty acid-based products, Nisin, glycerol monolaurate, quaternary ammonium compounds (e.g., alkyl dimethyl benzyl ammonium chlorite, alkyl dimethyl ethyl ammonium bromide). The barrier teat dip may be followed by cleaning prior to recolonization. For example, the cleaning may include aqueous ethanol, dodecylbenzenesulfonic acid (e.g., Opti Blue™ Teat Cleaner, DeLaval).

Sealants may include a teat sealant, e.g., bismuth subnitrate (e.g., Orbeseal®, Zoetis; Lockout™, Merial Boehringer Ingleheim), nonylphenol ethoxylate,

The suppression step—or decolonization—may be performed comprising administering 1-3 times daily, over a period of from 1 to 10 days; for example, on one, two, three, four, five, six, seven, eight, nine, ten, eleven, twelve, thirteen or fourteen days. In other embodiments, the suppression step may be administered from two, three, four, five, or six times, each administration from 6 to 48 hours, 8 to 40 hours, 18 to 36 hours, or about 20 to 28 hours apart. In specific embodiments, the suppression step is administered once per day from one to five, or three to four consecutive days. In some embodiments, the suppression step does not include systemic administration of antimicrobial agents. In some embodiments, the suppression step does not include systemic administration of antibiotic, antiviral, or antifungal agents. In other embodiments, the suppression step includes systemic administration of antimicrobial agents. In some embodiments, the suppression step may include systemic administration of one or more antibiotic, antiviral, or antifungal agents.

Replace

Methods are provided wherein an undesirable microorganism is durably replaced with a synthetic microorganism. The synthetic microorganism has the ability to fill the same ecological niche and/or may be of the same species, different strain, as the pathogenic microorganism. By using same species, different strain, (or even the same strain) the environmental niche of the pathogenic microorganism may be filled, or durably replaced, with the benign synthetic microorganism.

Synthetic Microorganism

In some embodiments, the undesirable pathogenic microorganism is replaced with a synthetic microorganism. For example, the replacement strain may be a synthetic microorganism that is a molecularly modified strain of the same species as the undesirable or pathogenic microorganism or the same strain as the undesirable or pathogenic microorganism.

In some embodiments, a synthetic microorganism comprising a “kill switch” is provided exhibiting rapid and complete cell death on exposure to blood or serum, but exhibits normal metabolism and colonization function in other environments. In some embodiments, the synthetic microorganism comprises stable and immobile kill switch genes. The minimal kill switch (KS) components include a regulatory region (RR) containing operator, promoter and translation signals, that is strongly activated in response to blood or serum exposure, a kill switch gene expressing a toxic protein or RNA, and a means of transcription termination. Chromosomal integration of the KS is preferred. The chromosomal locus may be in a transcriptionally inactive region, for example, an intergenic region (IR) between a seryl-tRNA synthetase and an amino acid transporter. Insertions here do not affect transcription of flanking genes (Lei et al., 2012). Preferably, no known sRNAs are present in the IR. Any other inert loci may be selected.

The Synthetic Microorganism Comprising a Kill Switch

In a particular embodiment, the pathogenic microorganism is an antimicrobial-resistant microorganism, and the replacement microorganism is a synthetic microorganism of the same species as the pathogenic microorganism. The synthetic microorganism may be a molecularly-modified, antibiotic-susceptible microorganism.

The synthetic microorganism may comprise one or more, two or more, or three or more molecular modifications comprising a first cell death gene operably linked to a first regulatory region comprising an inducible first promoter. Optionally, the synthetic microorganism further comprises a second cell death gene operably linked to the first regulatory region comprising the first promoter or a second regulatory region comprising an inducible second promoter. The first promoter, and optionally the second promoter, is activated (induced) by a change in state in the microorganism environment compared to the normal physiological conditions at the at least one site in the subject. For example, the change in state may be selected from one or more changes in pH, temperature, osmotic pressure, osmolality, oxygen level, nutrient concentration, blood concentration, plasma concentration, serum concentration, and electrolyte concentration. In some embodiments, the change in state is a higher concentration of blood, serum, or plasma compared to normal physiological conditions at the at least one site in the subject.

In one specific embodiment, the pathogenic microorganism is a MRSA and the replacement microorganism is a synthetic microorganism that is a molecularly modified Staphylococcus aureus coagulase positive strain. The synthetic microorganism may be a molecularly modified Staphylococcus aureus 502a, as described herein.

The use of live Staphylococcus aureus as a therapeutic platform raises safety concerns because this pathogen can cause serious disease if it gains access to the circulatory system. In one embodiment, the synthetic microorganism is molecularly engineered to comprise a “kill switch” (KS) and an inducible promoter that induces rapid bacterial death upon exposure to whole blood or serum. The kill switch may be composed of DNA encoding 3 main components: i) “control region”, containing a promoter and other regulatory sequences, that is strongly activated by blood or serum; ii) a toxic RNA or polypeptide, whose expression is driven by the control region, and; iii) a transcription terminator. A cassette composed of these elements maybe integrated into the Staphylococcus aureus chromosome at a site(s) amenable to alteration without adversely affecting bacterial function.

It is desirable that basal or “leaky” expression of the control region is minimized or avoided. For example, if significant mRNA production occurs before exposure to blood or serum, the strain could be weakened during manufacturing or skin colonization and may accumulate mutations that bypass or escape the KS. To address this, candidates are screened to find those that are strongly induced in serum, but also have very low or undetectable mRNA expression in standard growth media in vitro. Despite this effort, some leaky expression may be observed, which may be controlled by further comprising a iv) “expression clamp” to prevent untimely toxin production.

Recombinant Approach to Synthetic Microorganism

A synthetic microorganism is provided which comprises a recombinant nucleotide comprising at least one molecular modification (e.g., a kill switch) comprising (i) a cell death gene operatively associated with (ii) a first regulatory region comprising a first inducible promoter which is induced by a change in state in the environment of the synthetic microorganism. The synthetic microorganism may further comprises at least a second molecular modification (expression clamp) comprising (iii) an antitoxin gene specific for the first cell death gene, wherein the antitoxin gene is operably associated with (iv) a second regulatory region comprising a second promoter which is active (e.g., constitutive) upon dermal or mucosal colonization or in a media, and preferably is downregulated by change in state of the environment of the synthetic microorganism.

In some embodiments, a synthetic microorganism is provided comprising at least one molecular modification (e.g., a kill switch) comprising a first cell death gene operably linked to a first regulatory region comprising a first promoter, wherein the first promoter is activated (induced) by a change in state in the microorganism environment compared to the normal physiological conditions at the at least one site in the subject, optionally wherein cell death of the synthetic microorganism occurs within 30, 60, 90, 120, 180, 360 or 240 minutes following change of state. The change in state may be selected from one or more conditions of pH, temperature, osmotic pressure, osmolality, oxygen level, nutrient concentration, blood concentration, plasma concentration, serum concentration, heme concentration, sweat concentration, sebum concentration, metal concentration, chelated metal concentration, change in composition or concentration of one or more immune factors, mineral concentration, and electrolyte concentration. In some embodiments, the change in state is a higher concentration of blood, serum, or plasma compared to normal physiological conditions at the at least one site in the subject.

Inducible Promoters

A synthetic microorganism is provided which may comprise a recombinant nucleotide comprising at least one molecular modification (e.g., a kill switch) comprising (i) a cell death gene operatively associated with (ii) a first regulatory region comprising a first inducible promoter which exhibits conditionally high level gene expression of the recombinant nucleotide in response to exposure to blood, serum, or plasma, of at least two fold, at least three fold, at least 10-fold, at least 20 fold, at least 50 fold, at least 100-fold increase of basal productivity.

The inducible first promoter may be activated (induced) upon exposure to an increased concentration of blood, serum, plasma, or heme after a period of time, e.g., after 15 minutes, 30 minutes, 45 minutes, 90 minutes, 120 minutes, 180 minutes, 240 minutes, 360 minutes, or any time point in between, to increase transcription and/or expression at least 5-fold, at least 10-fold, at least 20-fold, at least 50-fold, at least 100-fold, at least 300-fold, or at least 600-fold compared to transcription and/or expression in the absence of blood, serum, plasma or heme (non-induced).

The blood or serum inducible first promoter may be selected by a process comprising selecting a target microorganism, selecting one or more first promoter candidate genes in the target microorganism, growing the microorganism in a media, obtaining samples of the microorganism at t=0 min, adding serum or blood to the media, obtaining samples at t=n minutes, where n=1-240 min or more, 15-180 min, or 30-120 min, performing RNA sequencing of the samples, and comparing RNA sequencing read numbers for candidate first promoter in samples obtained at obtained at t=0 min, and t=n minutes after exposure to blood or serum for the candidate first promoter gene. Alternatively, samples obtained after t=n minutes after exposure to blood or serum may be compared to t=n minutes in media without blood or serum for the candidate first promoter. Candidate first promoters may be selected from those that exhibit upregulation by RNA sequencing after target cell growth at t=n min in blood or serum of greater than about 10-fold, greater than about 20-fold, greater than about 50-fold, greater than about 100-fold, or greater than about 500-fold, when compared to the candidate promoter in the target cell at t=0, or when compared to the candidate promoter in the target cell at t=n in media without serum or blood.

Several serum responsive promoter candidate genes in Staphylococcus aureus 502a were upregulated by greater than 20-fold after exposure to serum for 30 minutes as determined by RNA sequencing as compared to t=0 including isdB gene CH52_00245 (479-fold), sbnB gene CH52_05135 (158-fold), isdC gene CH52_00235 (93-fold), sbnA gene CH52_05140 (88-fold), srtB gene CH52_00215 (73-fold), sbnE gene CH52_05120 (70-fold), sbnD gene CH52_05125 (66-fold), isdI gene CH52_00210 (65-fold), heme ABC transporter 2 gene CH52_00225 (65-fold), sbnC gene CH52_05130 (63-fold), heme ABC transporter gene CH52_00230 (60-fold), isd ORF3 gene CH52_00220 (51-fold), sbnF gene CH52_05115 (43 fold), alanine dehydrogenase gene CH52_11875 (43-fold), HarA gene CH52_10455 (43-fold), sbnG gene CH52_05110 (42-fold), diaminopimelate decarboxylase gene CH52_05105 (32-fold), iron ABC transporter gene CH52_05145 (31-fold), threonine dehydratase gene CH52_11880 (24-fold), and isdA gene CH52_00240 (21-fold).

Several serum responsive promoter candidate genes in target microorganism Staphylococcus aureus 502a were found to be upregulated by greater than 20-fold after exposure to serum for 30 minutes as determined by RNAseq compared to TSB at 30 minutes including isdB gene CH52_00245 (471-fold), isdC gene CH52_00235 (56-fold), isdI gene CH52_00210 (53-fold), sbnD gene CH52_05125 (52-fold), sbnC gene CH52_05130 (51-fold), sbnE gene CH52_05120 (50-fold), srtB gene CH52_00215 (47-fold), sbnB gene CH52_05135 (44-fold), sbnF gene CH52_05115 (44-fold), heme ABC transporter 2 gene CH52_00225 (43-fold), isdA gene CH52_00240 (40-fold), heme ABC transporter gene CH52_00230 (40-fold), sbnA gene CH52_05140 (37-fold), isd ORF3 gene CH52_00220 (35-fold), sbnG gene CH52_05110 (34-fold), HarA gene CH52_10455 (28-fold), diaminopimelate decarboxylase gene CH52_05105 (25-fold), sbnI gene CH52_05100 (22-fold), and alanine dehydrogenase gene CH52_11875 (20-fold). Iron ABC transporter gene CH52_05145 was upregulated (19-fold) after 30 min of exposure to serum compared to 30 min in TSB. Threonine dehydratase gene CH52_11880 was upregulated (14-fold) after 30 min of exposure to serum compared to 30 min in TSB.

Several serum responsive promoter candidate genes in target microorganism Staphylococcus aureus 502a were upregulated by greater than 50-fold after exposure to serum after 90 minutes as determined by RNAseq compared to t=0 including isdB gene CH52_00245 (2052-fold), sbnB gene CH52_05135 (310-fold), alanine dehydrogenase gene CH52_11875 (304-fold), sbnE gene CH52_05120 (190-fold), sbnD gene CH52_05125 (187-fold), isdC gene CH52_00235 (173-fold), sbnC gene CH52_05130 (162-fold), sbnA gene CH52_05140 (143-fold), srtB gene CH52_00215 (143-fold), sbnG gene CH52_05110 (133-fold), sbnF gene CH52_05115 (129-fold), heme ABC transporter gene CH52_00230 (125-fold), heme ABC transporter 2 gene CH52_00225 (117-fold), isdI gene CH52_00210 (115-fold), HarA gene CH52_10455 (114-fold), diaminopimelate decarboxylase gene CH52_05105 (102-fold), sbnI gene CH52_05100 (101-fold), isd ORF3 gene CH52_00220 (97-fold), SAM dep Metrans gene CH52_04385 (75-fold). Iron ABC transporter gene CH52_05145 (44-fold), isdA gene CH52_00240 (44-fold), and siderophore ABC transporter gene CH52_05150 (33-fold) were also upregulated after 90 min exposure to serum compared to t=0.

Several serum responsive promoter candidate genes in target microorganism Staphylococcus aureus 502a were found to be upregulated by greater than 50-fold after exposure to serum after 90 minutes as determined by RNA sequencing compared to growth in TSB at 90 minutes including isdB gene CH52_00245 (1240-fold), sbnD gene CH52_05125 (224-fold), heme ABC transporter gene CH52_00230 (196-fold), sbnE gene CH52_05120 (171-fold), srtB gene CH52_00215 (170-fold), isdC gene CH52_00235 (149-fold), sbnC gene CH52_05130 (147-fold), diaminopimelate decarboxylase gene CH52_05105 (141-fold), heme ABC transporter 2 gene CH52_00225 (135-fold), sbnB gene CH52_05135 (130-fold), sbnF gene CH52_05115 (127-fold), bnG gene CH52_05110 (120-fold), isd ORF3 gene CH52_00220 (119-fold), isdI gene CH52_00210 (118-fold), HarA gene CH52_10455 (117-fold), isdA gene CH52_00240 (115-fold), sbnA gene CH52_05140 (93-fold), and sbnI gene CH52_05100 (89-fold). Iron ABC transporter gene CH52_05145 (47-fold), siderophore ABC transporter gene CH52_05150 (37-fold), and SAM dep Metrans gene CH52_04385 (25-fold) were also upregulated after 90 min exposure to serum compared to TSB at t=90 min.

The blood or serum inducible first promoter genes for use in a Staphylococcus aureus synthetic microorganism may be selected from or derived from a gene selected from isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnE (lucA/lucC family siderophore biosynthesis protein), lrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), leuA (2-isopropylmalate synthase amino acid biosynthetic enzyme), sstA (iron transport membrane protein), sirA (iron ABC transporter substrate-binding protein), IsdA (heme transporter), and Spa (Staphyloccocal protein A), HlgA (gamma hemolysin), leuA (amino acid biosynthetic enzyme), sstA (iron transporter), sirA (iron transport), spa (protein A), or IsdA (heme transporter), or a substantially identical gene. The first promoter genes also may be selected from the group consisting of SAUSA300_0119 (Ornithine cyclodeaminase family protein), lrgA (Murein hydrolase transporter), and bioA (Adenosylmethionine-8-amino-7-oxononanoate aminotransferase), or a substantially identical gene.

The blood or serum blood or serum inducible first promoter genes for use in a Staphylococcus aureus synthetic microorganism may be selected from or derived from a gene selected from isdB gene CH52_00245, sbnD gene CH52_05125, heme ABC transporter gene CH52_00230, sbnE gene CH52_05120, srtB gene CH52_00215, isdC gene CH52_00235, sbnC gene CH52_05130, diaminopimelate decarboxylase gene CH52_05105, heme ABC transporter 2 gene CH52_00225, sbnB gene CH52_05135, sbnF gene CH52_05115, bnG gene CH52_05110, isd ORF3 gene CH52_00220, isdI gene CH52_00210, HarA gene CH52_10455, isdA gene CH52_00240, sbnA gene CH52_05140, and sbnI gene CH52_05100, iron ABC transporter gene CH52_05145, siderophore ABC transporter gene CH52_05150, and SAM dep Metrans gene CH52_04385.

The blood or serum inducible first promoter gene for use in a Staphylococcus aureus synthetic microorganism may be derived from or comprise a nucleotide sequence selected from 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical sequence.

In one embodiment, the synthetic microorganism is a molecularly modified Staphylococcus aureus 502a. Raw sequences of first ORF in the operon that follows each regulatory region, from start codon to stop codon, used for design of real time PCR probes are shown in Table 2.

TABLE 2
Staphylococcus aureus strain 502a, raw sequences of first ORF
in the operon that follows each regulatory region used for
design of real time PCR probes.

| | |
|---|---|
| Staphylococcus | ATGACTTTACAAATACATACAGGGGGTATTAATTT |
| aureus strain | GAAAAAGAAAAACATTTATTCAATTCGTAAACTAGGTGTAGGTATTGCAT |
| 502a, spa ORF of | CTGTAACTTTAGGTACATTACTTATATCTGGTGGCGTAACACCTGCTGCA |
| 502a | AATGCTGCGCAACACGATGAAGCTCAACAAAATGCTTTTTATCAAGTGTT |
| | AAATATGCCTAACTTAAACGCTGATCAACGTAATGGTTTTATCCAAAGCC |
| | TTAAAGATGATCCAAGCCAAAGTGCTAACGTTTTAGGTGAAGCTCAAAAA |
| | CTTAATGACTCTCAAGCTCCAAAAGCTGATGCGCAACAAAATAACTTCAA |
| | CAAAGATCAACAAAGCGCCTTCTATGAAATCTTGAACATGCCTAACTTAA |
| | ACGAAGCGCAACGTAACGGCTTCATTCAAAGTCTTAAAGACGACCCAAGC |
| | CAAAGCACTAATGTTTTAGGTGAAGCTAAAAAATTAAACGAATCTCAAGC |
| | ACCGAAAGCTGATAACAATTTCAACAAAGAACAACAAAATGCTTTCTATG |
| | AAATCTTGAATATGCCTAACTTAAACGAAGAACAACGCAATGGTTTCATC |
| | CAAAGCTTAAAAGATGACCCAAGCCAAAGTGCTAACCTATTGTCAGAAGC |
| | TAAAAAGTTAAATGAATCTCAAGCACCGAAAGCGGATAACAAATTCAACA |
| | AAGAACAACAAAATGCTTTCTATGAAATCTTACATTTACCTAACTTAAAC |
| | GAAGAACAACGCAATGGTTTCATCCAAAGCTTAAAAGATGACCCAAGCCA |
| | AAGCGCTAACCTTTTAGCAGAAGCTAAAAAGCTAAATGATGCACAAGCAC |
| | CAAAAGCTGACAACAAATTCAACAAAGAACAACAAAATGCTTTCTATGAA |
| | ATTTTACATTTACCTAACTTAACTGAAGAACAACGTAACGGCTTCATCCA |
| | AAGCCTTAAAGACGATCCTTCAGTGAGCAAAGAAATTTTAGCAGAAGCTA |
| | AAAAGCTAAACGATGCTCAAGCACCAAAAGAGGAAGACAACAAAAAACCT |
| | GGTAAAGAAGACGGCAACAAGCCTGGTAAAGAAGACAACAAAAAACCTGG |
| | TAAAGAAGACGGCAACAAGCCTGGTAAAGAAGACAACAACAAACCTGGCA |
| | AAGAAGACGGCAACAAGCCTGGTAAAGAAGACAACAACAAGCCTGGTAAA |
| | GAAGACGGCAACAAGCCTGGTAAAGAAGACGGCAACAAACCTGGTAAAGA |
| | AGACGGCAACGGAGTACATGTCGTTAAACCTGGTGATACAGTAAATGACA |
| | TTGCAAAAGCAAACGGCACTACTGCTGACAAAATTGCTGCAGATAACAAA |
| | TTAGCTGATAAAAACATGATCAAACCTGGTCAAGAACTTGTTGTTGATAA |
| | GAAGCAACCAGCAAACCATGCAGATGCTAACAAAGCTCAAGCATTACCAG |
| | AAACTGGTGAAGAAAATCCATTCATCGGTACAACTGTATTTGGTGGATTA |
| | TCATTAGCCTTAGGTGCAGCGTTATTAGCTGGACGTCGTCGCGAACTATA |
| | A |
| | SEQ ID NO: 1 |
| Staphylococcus | ATGAATAAAGTAATTAAAATGC |
| aureus strain | TTGTTGTTACGCTTGCTTTCCTACTTGTTTTAGCAGGATGTAGTGGGAAT |
| 502a, sirA ORF of | TCAAATAAACAATCATCTGATAACAAAGATAAGGAAACAACTTCAATTAA |
| 502a | ACATGCAATGGGTACAACTGAAATTAAAGGGAAACCAAAGCGTGTTGTTA |
| | CGCTATATCAAGGTGCCACTGACGTCGCTGTATCTTTAGGTGTTAAACCT |
| | GTAGGTGCTGTAGAATCATGGACACAAAAACCGAAATTCGAATACATAAA |
| | AAATGATTTAAAAGATACTAAGATTGTAGGTCAAGAACCTGCACCTAACT |
| | TAGAGGAAATCTCTAAATTAAAACCGGACTTAATTGTCGCGTCAAAAGTT |
| | AGAAATGAAAAAGTTTACGATCAATTATCTAAAATCGCACCAACAGTTTC |
| | TACTGATACAGTTTTCAAATTCAAAGATACAACTAAGTTAATGGGGAAAG |
| | CTTTAGGGAAAGAAAAAGAAGCTGAAGATTTACTTAAAAAGTACGATGAT |
| | AAAGTAGCTGCATTCCAAAAAGATGCAAAAGCAAAGTATAAAGATGCATG |
| | GCCATTGAAAGCTTCAGTTGTTAACTTCCGTGCTGATCATACAAGAATTT |
| | ATGCTGGTGGATATGCTGGTGAAATCTTAAATGATTTAGGATTCAAACGT |
| | AATAAAGACTTACAAAAACAAGTTGATAATGGTAAAGATATTATCCAACT |
| | TACATCTAAAGAAAGCATTCCATTAATGAACGCTGATCATATTTTTGTAG |
| | TAAAATCAGATCCAAATGCGAAAGATGCTGCATTAGTTAAAAAGACTGAA |
| | AGCGAATGGACTTCAAGTAAAGAGTGGAAAAATTTAGACGCAGTTAAAAA |
| | CAACCAAGTATCTGATGATTTAGATGAAATCACTTGGAACTTAGCTGGCG |
| | GATATAAATCTTCATTAAAACTTATTGACGATTTATATGAAAAGTTAAAT |
| | ATTGAAAAACAATCAAAATAA |
| | SEQ ID NO: 2 |
| Staphylococcus | ATGATAATGATTATCATTAATTTAA |
| aureus strain | AGGGAGAAAAATTTGTAATGAAGTATTTATTAAAGGGAAATATTTTGCTT |
| 502a, sstA of 502a | CTATTACTAATATTGTTGACAATTATTTCGTTGTTCATAGGTGTGAGTGA |
| | ACTATCAATTAAAGATTTACTACATTTAACTGAATCACAGCGGAATATTT |
| | TATTCTCAAGCCGAATACCAAGGACGATGAGTATTTTAATTGCTGGAAGT |
| | TCGTTGGCTTTAGCAGGCTTGATAATGCAACAAATGATGCAAAATAAGTT |
| | TGTTAGTCCGACTACAGCTGGAACGATGGAATGGGCTAAACTAGGTATTT |
| | TAATTGCTTTATTGTTCTTTCCAACCGGTCATATTTTATTAAAACTAGTA |
| | TTTGCTGTTATTTGCAGTATTTGCGGTACGTTTTTATTTGTTAAAATCAT |
| | TGATTTTATAAAAGTGAAAGATGTCATTTTTGTACCGCTTTTAGGAATTA |
| | TGATGGGTGGGATTGTTGCAAGTTTCACAACCTTCATCTCATTGCGCACG |
| | AATGCTGTTCAAAGCATTGGTAACTGGCTTAACGGGAACTTTGCCATTAT |
| | CACAAGTGGACGCTATGAAATTTTATATTTAAGTATTCCTCTTTTAGCAT |
| | TGACATATCTTTTTGCTAATCATTTCACGATTGTAGGAATGGGTAAAGAC |
| | TTTACTAATAATTTAGGTTTGAGTTACGAAAAATTAATTAACATCGCATT |
| | GTTTATTACTGCAACTATTACAGCATTGGTAGTGGTGACTGTTGGAACAT |
| | TACCGTTCTTAGGACTAGTAATACCAAATATTATTTCAATTTATCGAGGT |
| | GATCATTTGAAAAATGCTATCCCTCATACGATGATGTTAGGTGCCATCTT |
| | TGTATTATTTTCTGATATAGTTGGCAGAATTGTTGTTTATCCATATGAAA |
| | TAAATATTGGTTTAACAATAGGTGTATTTGGAACAATCATTTTCCTTATC |
| | TTGCTTATGAAAGGTAGGAAAAATTATGCGCAACAATAA |
| | SEQ ID NO: 3 |
| Staphylococcus | ATGAACT |
| aureus strain | TAAAATTAAATAGAAAGAAAGTGATTTCTATGATTAAAAATAAAATATTA |
| 502a, hlgA ORF | ACAGCAACTTTAGCAGTTGGTTTAATAGCCCCTTTAGCCAATCCATTTAT |
| of 502a | AGAAATTTCTAAAGCAGAAAATAAGATAGAAGATATCGGTCAAGGTGCAG |
| | AAATCATCAAAAGAACACAAGACATTACTAGCAAACGATTAGCTATAACT |
| | CAAAACATTCAATTTGATTTTGTAAAAGATAAAAAATATAACAAAGATGC |
| | CCTAGTTGTTAAGATGCAAGGCTTCATCAGCTCTAGAACAACATATTCAG |
| | ACTTAAAAAAATATCCATATATTAAAAGAATGATATGGCCATTTCAATAT |
| | AATATCAGTTTGAAAACGAAAGACTCTAATGTTGATTTAATCAATTATCT |
| | TCCTAAAAATAAAATTGATTCAGCAGATGTTAGTCAGAAATTAGGCTATA |
| | ATATCGGCGGAAACTTCCAATCAGCGCCATCAATCGGAGGCAGTGGCTCA |
| | TTCAACTACTCTAAAACAATTAGTTATAATCAAAAAAACTATGTTACTGA |
| | AGTAGAAAGTCAGAACTCTAAAGGTGTTAAATGGGGAGTGAAAGCAAATT |
| | CATTTGTTACACCGAATGGTCAAGTATCTGCATATGATCAATACTTATTT |
| | GCACAAGACCCAACTGGTCCAGCAGCAAGAGACTATTTCGTCCCAGATAA |
| | TCAATTACCTCCTTTAATTCAAAGTGGCTTTAATCCATCATTTATTACAA |
| | CATTGTCACACGAAAGAGGTAAAGGTGATAAAAGCGAGTTTGAAATCACT |
| | TACGGCAGAAACATGGATGCTACATATGCTTACGTGACAAGACATCGTTT |
| | AGCCGTTGATAGAAAACATGATGCTTTTAAAAACCGAAACGTTACAGTTA |
| | AATATGAAGTGAACTGGAAAACACATGAAGTAAAAATTAAAAGCATCACA |
| | CCTAAGTAA |
| | SEQ ID NO: 4 |
| | ATGACAAAACATTATTTAAACAGTAAGTATCAATC |
| Staphylococcus | AGAACAACGTTCATCAGCTATGAAAAAGATTACAATGGGTACAGCATCTA |
| aureus strain | TCATTTTAGGTTCCCTTGTATACATAGGCGCAGACAGCCAACAAGTCAAT |
| 502a, isdA ORF | GCGGCAACAGAAGCTACGAACGCAACTAATAATCAAAGCACACAAGTTTC |
| of 502a | TCAAGCAACATCACAACCAATTAATTTCCAAGTGCAAAAAGATGGCTCTT |
| | CAGAGAAGTCACACATGGATGACTATATGCAACACCCTGGTAAAGTAATT |
| | AAACAAAATAATAAATATTATTTCCAAACCGTGTTAAACAATGCATCATT |
| | CTGGAAAGAATACAAATTTTACAATGCAAACAATCAAGAATTAGCAACAA |
| | CTGTTGTTAACGATAATAAAAAAGCGGATACTAGAACAATCAATGTTGCA |
| | GTTGAACCTGGATATAAGAGCTTAACTACTAAAGTACATATTGTCGTGCC |
| | ACAAATTAATTACAATCATAGATATACTACGCATTTGGAATTTGAAAAAG |
| | CAATTCCTACATTAGCTGACGCAGCAAAACCAAACAATGTTAAACCGGTT |
| | CAACCAAAACCAGCTCAACCTAAAACACCTACTGAGCAAACTAAACCAGT |
| | TCAACCTAAAGTTGAAAAAGTTAAACCTACTGTAACTACAACAAGCAAAG |
| | TTGAAGACAATCACTCTACTAAAGTTGTAAGTACTGACACAACAAAAGAT |
| | CAAACTAAAACACAAACTGCTCATACAGTTAAAACAGCACAAACTGCTCA |
| | AGAACAAAATAAAGTTCAAACACCTGTTAAAGATGTTGCAACAGCGAAAT |
| | CTGAAAGCAACAATCAAGCTGTAAGTGATAATAAATCACAACAAACTAAC |
| | AAAGTTACAAAACATAACGAAACGCCTAAACAAGCATCTAAAGCTAAAGA |
| | ATTACCAAAAACTGGTTTAACTTCAGTTGATAACTTTATTAGCACAGTTG |
| | CCTTCGCAACACTTGCCCTTTTAGGTTCATTATCTTTATTACTTTTCAAA |
| | AGAAAAGAATCTAAATAA |
| | SEQ ID NO: 5 |
| Staphylococcus | ATGAGTAGTCATATTCAAATTTTTGATACGACACTAAGAGACGGTGAACA |
| aureus strain | AACACCAGGAGTGAATTTTACTTTTGATGAACGCTTGCGTATTGCATTGC |
| 502a, leuA of | AATTAGAAAAATGGGGTGTAGATGTTATTGAAGCTGGATTTCCTGCTTCA |
| 502a | AGTACAGGTAGCTTTAAATCTGTTCAAGCAATTGCACAAACATTAACAAC |
| | AACGGCTGTATGTGGTTTAGCTAGATGTAAAAAATCTGACATCGATGCTG |
| | TATATGAAGCAACAAAAGATGCAGCGAAGCCGGTCGTGCATGTTTTTATA |
| | GCAACATCACCTATTCATCTTGAACATAAACTTAAAATGTCTCAAGAAGA |
| | CGTTTTAGCATCTATTAAAGAACATGTCACATACGCGAAACAATTATTTG |
| | ACGTTGTTCAATTTTCACCTGAAGATGCAACGCGTACTGAATTACCATTC |
| | TTAGTGAAATGTGTACAAACTGCCGTTGACGCTGGAGCTACAGTTATTAA |
| | TATTCCTGATACAGTCGGCTACAGTTACCATGATGAATATGCACATATTT |
| | TCAAAACCTTAACAGAATCTGTAACATCTTCAAATGAAATTATTTATAGT |
| | GCTCATTGCCATGACGATTTAGGAATGGCTGTTTCAAATAGTTTAGCTGC |
| | AATTGAAGGCGGTGCGAGACGAATTGAAGGCACTGTAAATGGTATTGGTG |
| | AACGAGCAGGTAATGCAGCACTTGAAGAAGTCGCGCTTGCACTATACGTT |
| | CGAAATGATCATTATGGTGCTCAAACTGCCCTTAATCTCGAAGAAACTAA |
| | AAAAACATCGGATTTAATTTCAAGATATGCAGGTATTCGAGTGCCTAGAA |
| | ATAAAGCAATTGTTGGCCAAAATGCATTTAGTCATGAATCAGGTATTCAC |
| | CAAGATGGCGTATTAAAACATCGTGAAACATATGAAATTATGACACCTCA |
| | ACTTGTTGGTGTAAGCACGACTGAACTTCCATTAGGAAAATTATCTGGTA |
| | AACACGCCTTCTCAGAGAAGTTAAAAGCATTAGGTTATAACATTGATAAA |
| | GAAGCGCAAATAGATTTATTTAAACAATTCAAGACCATTGCGGACAAAAA |
| | GAAATCTGTTTCAGATAGAGATATTCATGCGATTATTCAAGGTTCTGAGC |
| | ATGAGCATCAAGCACTTTATAAATTGGAAACACTACAACTACAATATGTC |
| | TCTAGCGGCCTTCAAAGTGCTGTTGTTGTTGTTAAAGATAAAGAGGGTCA |
| | TATTTACCAGGATTCAAGTATTGGTACTGGTTCAATCGTAGCAATTTACA |
| | ATGCAGTTGATCGTATTTTCCAGAAAGAAACAGAATTAATTGATTATCGT |
| | ATTAATTCTGTCACTGAAGGTACTGATGCCCAAGCAGAAGTACATGTAAA |
| | TTTATTGATTGAAGGTAAGACTGTCAATGGCTTTGGTATTGATCATGATA |
| | TTTTACAAGCCTCTTGTAAAGCATACGTAGAAGCACATGCTAAATTTGCA |
| | GCTGAAAATGTTGAGAAGGTAGGTAAT |
| | SEQ ID NO: 6 |

As discussed herein below, the synthetic microorganism may include an expression clamp molecular modification that prevents expression of the cell death gene, wherein the expression clamp comprises an antitoxin gene specific for the cell death gene operably associated with a second promoter which is active upon dermal or mucosal colonization or in TSB media, and is preferably downregulated in blood, serum or plasma, for example, the second promoter may comprise a clfB gene (clumping factor B), for example as shown in Table 3.

TABLE 3
Other Sequences Used for Design of Real time PCR probes

| | |
|---|---|
| clfB ORF of 502a | ATGAAAAAAAGAATTGATTATTTGTCGAATAAGCAGAATAAGTATTCGAT |
| (to drive antitoxin | TAGACGTTTTACAGTAGGTACCACATCAGTAATAGTAGGGGCAACTATAC |
| for “expression | TATTTGGGATAGGCAATCATCAAGCACAAGCTTCAGAACAATCGAACGAT |
| clamp”) | ACAACGCAATCTTCGAAAAATAATGCAAGTGCAGATTCCGAAAAAAACAA |
| | TATGATAGAAACACCTCAATTAAATACAACGGCTAATGATACATCTGATA |
| | TTAGTGCAAACACAAACAGTGCGAATGTAGATAGCACAACAAAACCAATG |
| | TCTACACAAACGAGCAATACCACTACAACAGAGCCAGCTTCAACAAATGA |
| | AACACCTCAACCGACGGCAATTAAAAATCAAGCAACTGCTGCAAAAATGC |
| | AAGATCAAACTGTTCCTCAAGAAGCAAATTCTCAAGTAGATAATAAAACA |
| | ACGAATGATGCTAATAGCATAGCAACAAACAGTGAGCTTAAAAATTCTCA |
| | AACATTAGATTTACCACAATCATCACCACAAACGATTTCCAATGCGCAAG |
| | GAACTAGTAAACCAAGTGTTAGAACGAGAGCTGTACGTAGTTTAGCTGTT |
| | GCTGAACCGGTAGTAAATGCTGCTGATGCTAAAGGTACAAATGTAAATGA |
| | TAAAGTTACGGCAAGTAATTTCAAGTTAGAAAAGACTACATTTGACCCTA |
| | ATCAAAGTGGTAACACATTTATGGCGGCAAATTTTACAGTGACAGATAAA |
| | GTGAAATCAGGGGATTATTTTACAGCGAAGTTACCAGATAGTTTAACTGG |
| | TAATGGAGACGTGGATTATTCTAATTCAAATAATACGATGCCAATTGCAG |
| | ACATTAAAAGTACGAATGGCGATGTTGTAGCTAAAGCAACATATGATATC |
| | TTGACTAAGACGTATACATTTGTCTTTACAGATTATGTAAATAATAAAGA |
| | AAATATTAACGGACAATTTTCATTACCTTTATTTACAGACCGAGCAAAGG |
| | CACCTAAATCAGGAACATATGATGCGAATATTAATATTGCGGATGAAATG |
| | TTTAATAATAAAATTACTTATAACTATAGTTCGCCAATTGCAGGAATTGA |
| | TAAACCAAATGGCGCGAACATTTCTTCTCAAATTATTGGTGTAGATACAG |
| | CTTCAGGTCAAAACACATACAAGCAAACAGTATTTGTTAACCCTAAGCAA |
| | CGAGTTTTAGGTAATACGTGGGTGTATATTAAAGGCTACCAAGATAAAAT |
| | CGAAGAAAGTAGCGGTAAAGTAAGTGCTACAGATACAAAACTGAGAATTT |
| | TTGAAGTGAATGATACATCTAAATTATCAGATAGCTACTATGCAGATCCA |
| | AATGACTCTAACCTTAAAGAAGTAACAGACCAATTTAAAAATAGAATCTA |
| | TTATGAGCATCCAAATGTAGCTAGTATTAAATTTGGTGATATTACTAAAA |
| | CATATGTAGTATTAGTAGAAGGGCATTACGACAATACAGGTAAGAACTTA |
| | AAAACTCAGGTTATTCAAGAAAATGTTGATCCTGTAACAAATAGAGACTA |
| | CAGTATTTTCGGTTGGAATAATGAGAATGTTGTACGTTATGGTGGTGGAA |
| | GTGCTGATGGTGATTCAGCAGTAAATCCGAAAGACCCAACTCCAGGGCCG |
| | CCGGTTGACCCAGAACCAAGTCCAGACCCAGAACCAGAACCAACGCCAGA |
| | TCCAGAACCAAGTCCAGACCCAGAACCGGAACCAAGCCCAGACCCGGATC |
| | CGGATTCGGATTCAGACAGTGACTCAGGCTCAGACAGCGACTCAGGTTCA |
| | GATAGCGACTCAGAATCAGATAGCGATTCGGATTCAGACAGTGATTCAGA |
| | TTCAGACAGCGACTCAGAATCAGATAGCGATTCAGAATCAGATAGCGACT |
| | CAGATTCAGATAGCGATTCAGATTCAGATAGCGATTCAGAATCAGATAGC |
| | GATTCGGATTCAGACAGTGATTCAGATTCAGACAGCGACTCAGAATCAGA |
| | TAGCGACTCAGAATCAGATAGTGAGTCAGATTCAGACAGTGACTCGGACT |
| | CAGACAGTGATTCAGACTCAGATAGCGATTCAGACTCAGATAGCGATTCA |
| | GACTCAGACAGCGATTCAGATTCAGACAGCGACTCAGAATCAGACAGCGA |
| | CTCAGACTCAGATAGCGACTCAGACTCAGACAGCGACTCAGATTCAGATA |
| | GCGATTCAGACTCAGACAGCGACTCAGACTCAGACAGCGACTCAGACTCA |
| | GATAGCGATTCAGACTCAGACAGCGACTCAGATTCAGATAGCGATTCGGA |
| | CTCAGACAGCGATTCAGATTCAGACAGCGACTCAGACTCGGATAGCGATT |
| | CAGATTCAGACAGCGACTCAGACTCGGATAGCGACTCGGATTCAGATAGT |
| | GACTCCGATTCAAGAGTTACACCACCAAATAATGAACAGAAAGCACCATC |
| | AAATCCTAAAGGTGAAGTAAACCATTCTAATAAGGTATCAAAACAACACA |
| | AAACTGATGCTTTACCAGAAACAGGAGATAAGAGCGAAAACACAAATGCA |
| | ACTTTATTTGGTGCAATGATGGCATTATTAGGATCATTACTATTGTTTAG |
| | AAAACGCAAGCAAGATCATAAAGAAAAAGCGTAAATACTTTTTTAGGCCG |
| | AATACATTTGTATTCGGTTTTTTTGTTGAAAATGATTTTAAAGTGAATTG |
| | SEQ ID NO: 7 |
| gyrA ORF of 502a | ATGGCTGAATTACCTCAATCAAGAATAAATGAACGAAATATTACCAGTGA |
| (internal | AATGCGTGAATCATTTTTAGATTATGCGATGAGTGTTATCGTTGCTCGTG |
| housekeeping | CATTGCCAGATGTTCGTGACGGTTTAAAACCAGTACATCGTCGTATACTA |
| gene) | TATGGATTAAATGAACAAGGTATGACACCGGATAAATCATATAAAAAATC |
| | AGCACGTATCGTTGGTGACGTAATGGGTAAATATCACCCTCATGGTGACT |
| | CATCTATTTATGAAGCAATGGTACGTATGGCTCAAGATTTCAGTTATCGT |
| | TATCCGCTTGTTGATGGCCAAGGTAACTTTGGTTCAATGGATGGAGATGG |
| | CGCAGCAGCAATGCGTTATACTGAAGCGCGTATGACTAAAATCACACTTG |
| | AACTGTTACGTGATATTAATAAAGATACAATAGATTTTATCGATAACTAT |
| | GATGGTAATGAAAGAGAGCCGTCAGTCTTACCTGCTCGATTCCCTAACTT |
| | GTTAGCCAATGGAGCATCAGGTATAGCGGTAGGTATGGCAACGAATATTC |
| | CACCACATAACTTAACAGAATTAATCAATGGTGTACTTAGCTTAAGTAAG |
| | AACCCTGATATTTCAATTGCTGAGTTAATGGAGGATATTGAAGGTCCTGA |
| | TTTCCCAACTGCTGGACTTATTTTAGGTAAGAGTGGTATTAGACGTGCAT |
| | ATGAAACAGGTCGTGGTTCAATTCAAATGCGTTCTCGTGCAGTTATTGAA |
| | GAACGTGGAGGCGGACGTCAACGTATTGTTGTCACTGAAATTCCTTTCCA |
| | AGTGAATAAGGCTCGTATGATTGAAAAAATTGCAGAGCTCGTTCGTGACA |
| | AGAAAATTGACGGTATCACTGATTTACGTGATGAAACAAGTTTACGTACT |
| | GGTGTGCGTGTCGTTATTGATGTGCGTAAGGATGCAAATGCTAGTGTCAT |
| | TTTAAATAACTTATACAAACAAACACCTCTTCAAACATCATTTGGTGTGA |
| | ATATGATTGCACTTGTAAATGGTAGACCGAAGCTTATTAATTTAAAAGAA |
| | GCGTTGGTACATTATTTAGAGCATCAAAAGACAGTTGTTAGAAGACGTAC |
| | GCAATACAACTTACGTAAAGCTAAAGATCGTGCCCACATTTTAGAAGGAT |
| | TACGTATCGCACTTGACCATATCGATGAAATTATTTCAACGATTCGTGAG |
| | TCAGATACAGATAAAGTTGCAATGGAAAGCTTGCAACAACGCTTCAAACT |
| | TTCTGAAAAACAAGCTCAAGCTATTTTAGACATGCGTTTAAGACGTCTAA |
| | CAGGTTTAGAGAGAGACAAAATTGAAGCTGAATATAATGAGTTATTAAAT |
| | TATATTAGTGAATTAGAAACAATCTTAGCTGATGAAGAAGTATTACTACA |
| | ATTAGTTAGAGATGAATTAACAGAAATTCGAGATCGTTTCGGTGATGATC |
| | GTCGTACTGAAATCCAATTAGGTGGATTTGAAGATTTAGAAGATGAAGAT |
| | CTCATTCCAGAAGAACAAATTGTAATTACACTAAGCCATAATAACTACAT |
| | TAAACGTTTGCCGGTATCTACATATCGTGCTCAAAACCGTGGTGGTCGTG |
| | GTGTTCAAGGTATGAATACATTGGAAGAAGATTTTGTCAGTCAATTGGTA |
| | ACTTTAAGTACACATGACCATGTATTGTTCTTTACTAACAAAGGTCGTGT |
| | ATACAAACTTAAAGGTTATGAAGTGCCTGAGTTATCAAGACAGTCTAAAG |
| | GTATTCCTGTAGTGAATGCTATTGAACTTGAAAATGATGAAGTCATTAGT |
| | ACAATGATTGCTGTTAAAGACCTTGAAAGTGAAGACAACTTCTTAGTGTT |
| | TGCAACTAAACGTGGTGTCGTTAAACGTTCAGCATTAAGTAACTTCTCAA |
| | GAATAAATAGAAATGGTAAGATTGCGATTTCGTTCAGAGAAGATGATGAG |
| | TTAATTGCAGTTCGCTTAACAAGTGGTCAAGAAGATATCTTGATTGGTAC |
| | ATCACATGCATCATTAATTCGATTCCCTGAATCAACATTACGTCCTTTAG |
| | GCCGTACAGCAACGGGTGTGAAAGGTATTACACTTCGTGAAGGTGACGAA |
| | GTTGTAGGGCTTGATGTAGCTCATGCAAACAGTGTTGATGAAGTATTAGT |
| | AGTTACTGAAAATGGTTATGGTAAACGTACGCCAGTTAATGACTATCGTT |
| | TATCAAATCGTGGTGGTAAAGGTATTAAAACAGCTACGATTACTGAGCGT |
| | AATGGTAATGTTGTATGTATCACTACAGTAACTGGTGAAGAAGATTTAAT |
| | GATTGTTACTAATGCAGGTGTCATTATTCGACTAGATGTTGCAGATATTT |
| | CTCAAAATGGTCGTGCAGCACAAGGTGTTCGCTTAATTCGCTTAGGTGAT |
| | GATCAATTTGTTTCAACGGTTGCTAAAGTAAAAGAAGATGCAGAAGATGA |
| | AACGAATGAAGATGAGCAATCTACTTCAACTGTATCTGAAGATGGTACTG |
| | AACAACAACGTGAAGCGGTTGTAAATGATGAAACACCAGGAAATGCAATT |
| | CATACTGAAGTGATTGATTCAGAAGAAAATGATGAAGATGGACGTATTGA |
| | AGTAAGACAAGATTTCATGGATCGTGTTGAAGAAGATATACAACAATCAT |
| | CAGATGAAGATGAAGAATAATAA |
| | SEQ ID NO: 8 |

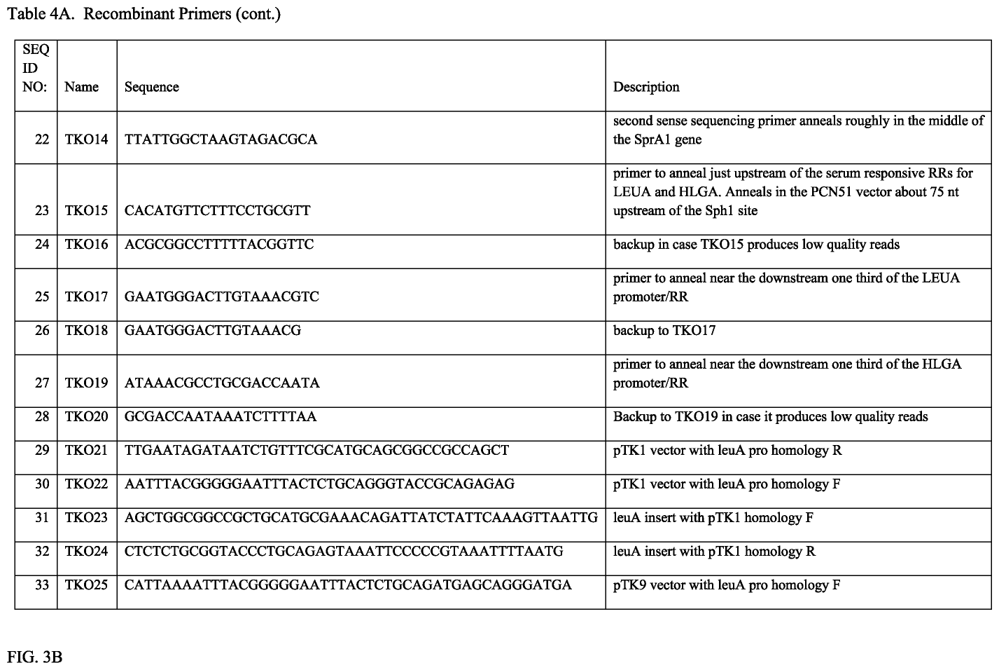

Additional oligonucleotides used in the recombinant approach to preparing the synthetic microorganism molecularly modified Staphylococcus aureus 502a are shown in Table 4A shown in FIG. 3A-C, and promoter sequences are shown below.

Cell Death Genes

The synthetic microorganism may contain a kill switch molecular modification comprising cell death gene operably associated with an inducible first promoter, as described herein. The cell death gene may be selected from any gene, that upon overexpression results in cell death or significant reduction in the growth of the synthetic microorganism within a predefined period of time, preferably within 15 minutes, 30 minutes, 60 minutes, 90 minutes, 120 minutes, 240 minutes, or 360 minutes of induction.

Cell death genes, toxin genes, or kill switch genes, have been developed in other contexts.

WO 2016/210373, Jonathan Kotula et al., assigned to Synlogic, Inc. discloses a recombinant bacterial cell that is an auxotroph engineered for biosafety, for example, that comprises a repression based kill switch gene that comprises a toxin, an anti-toxin and an arabinose inducible promoter and depends on the presence of an inducer (e.g., arabinose) to keep cells alive.

U.S. Pat. No. 8,975,061, Bielinski, discloses regulation of toxin and antitoxin genes for biological containment for preventing unintentional and/or uncontrolled spread of the microorganisms in the environment.

WO 1999/058652, Gerdes, discloses cytotoxin based biological containment and kill systems including E. coli relBE locus and similar systems found in Gram-negative and Gram-positive bacteria and Archea.

US 20150050253, Gabant, discloses controlled growth of microorganisms and controlling the growth/spread of other exogenous recombinant or other microbes.

WO 2017/023818 and WO 2016/210384, Falb, disclose bacteria engineered to treat disorders involving propionate metabolism.

US 20160333326, Falb, discloses bacteria engineered to treat diseases associated with hyperammonemia.

U.S. Pat. No. 9,101,597, Garry, discloses immunoprotective primary mesenchymal stem cells and methods and a proaptoptotic kill switch is described for use in mesenchymal stem cells.

US 20160206666, Falb, discloses bacteria engineered to treat diseases that benefit from reduced gut inflammation and/or tighten gut mucosal barrier.

In some embodiments, synthetic microorganisms are provided that comprise one or more of SprA1 (Staphylococcus aureus), Sma1 (Serratia marcescens), RelF (E. coli), KpnI (K. pneumoniae) and/or RsaE (Staphylococcus aureus) toxin genes.

In the present disclosure, various cell death toxin genes were tested in combinations with previously identified optimal control regions: i) a 30 amino acid peptide (PepA1) that forms pores in the cell membrane, impairing its function; ii) a restriction enzyme (Kpn1 or other) that rapidly digests the bacterial chromosome; iii) a small RNA (RsaE) that impairs central biochemical metabolism by inhibiting translation of 2 essential genes; iv) a restriction endonuclease (Sma1) derived from Serratia marcescens; and v) a toxin gene derived from E. coli (RelF). Some toxins are more potent than others and the ideal combination of control region induction strength and toxin potency may result in a strain that is healthy at baseline and that rapidly dies in the circulatory system.

sprA1 (Staphylococcus aureus) toxin gene (encoding PepA1 peptide) is described in WO 2013/050590, Felden, B, and Sayed, N, disclosing use of PepA1 as an antimicrobial, but the focus is on using the peptide as purified exogenous therapeutic to be delivered into the body.

relF (E. coli) toxin gene is described in EP 20090168998, Gerdes, disclosing kill switches for the purpose of biocontainment and focuses on revolve around killing of Gram-negative bacteria.

relF toxin gene is described in U.S. Pat. No. 8,852,916, Hyde and Roderick, disclosing mechanisms of triggering cell death of microorganisms (programmed cell death). The main application is to use RelF in environmental biocontainment.

relF is described in U.S. Pat. No. 8,682,619, Amodei, prophetically discloses RelF to regulate bacterial population growth.

The synthetic microorganism may be derived from a Staphylococcus aureus target microorganism by insertion of a kill switch molecular modification comprising a regulatory region comprising an inducible promoter operably linked to a cell death gene which may be a toxin gene.

The cell death gene may be selected from or derived from a sprA1 gene (encoding a peptide toxin that forms pores in cell membrane), sprA2 gene, sprG gene, sma1 gene (a restriction endonuclease), kpn1 gene (restriction enzyme that rapidly digests bacterial chromosome), rsaE gene (a small RNA that impairs central metabolism by inhibiting translation of 2 essential genes), a relF gene (E. co/i), yoeB gene, mazF gene, yefM gene, or lysostaphin toxin gene. The synthetic Staphylococcus aureus may include a kill switch molecular modification comprising a cell death gene having a nucleotide sequence selected from SEQ ID NOs: 122, 124, 125, 126, 127, 128, 274, 275, 284, 286, 288, 290, 315, or 317, or a substantially identical nucleotide sequence.

In a specific embodiment, a synthetic Staphylococcus aureus is provided having a molecular modification comprising a blood or serum inducible first promoter operably associated with a cell death gene comprising or derived from a SprA1 gene.

Multiple Kill Switches

One KS may be sufficient to equip the synthetic microorganism with the desired characteristics, but more than one KS may further enhance the strain by: i) dramatically reducing the rate of KS-inactivating mutations, and; ii) killing the cell by more than one pathway, which could cause faster cell death (a product-enhancing feature). The cell death gene may comprise one or more of the DNA sequences (7) downstream of promoters that are shown below. Base pair numbers correspond to pCN51 vector location.

1. The sprA1 gene sequence between restriction sites PstI and EcoRI is shown below. The sequence was synthesized by DNA 2.0(Atum) and ligated into a vector, which can be transformed into E. coli cells for replication. The sprA1 gene was restriction cut at PstI and EcoRI sites and isolated by gel electrophoresis. Full sequence between restriction sites with possible start and stop sites italicized.

| SEQ ID NO: 122 | |
|---|---|
| PstI | |
| | CTGCAGGG TACCGCAGAG AGGAGGTGTA |
| 6101 | TAAGGTG [symbol] CTTATTTTCG TTCACATCAT AGCACCAGTC ATCAGTGGCT |
| 6151 | GTGCCATTGC GTTTTTTTCT TATTGGCTAA GTAGACGCAA TACAAAA [symbol] |
| 6201 | GTGACATATA GCCGCACCAA TAAAAATCCC CTCACTACCG CAAATAGTGA |
| 6251 | GGGGATTGGT GTATAAGTAA ATACTTATTT TCGTTGTGGA TCCTTGACTG |
| 6301 | AATTC |

EcoRI

2. The DNA sequence for the regulatory RNA sprA1sprA1_{AS} (sprA1sprA1 antisense) under the ClfB promoter (which is cloned in reverse behind the sprA1 gene, including the antisense regulatory RNA). This DNA sequence produces a non-coding antisense regulatory RNA, which acts as an antitoxin by regulating the translation of sprA1 outside of the environmental factors of serum and/or blood. Below is the sprA1sprA1_{AS} DNA sequence.

SEQ ID NO: 123
EcoRI
GAATTCAGTCAAGGATCCACAACGAAAATAAGTATTTACTTATACACCA
ATCCCCTCACTATTTGCGGTAGTGAGGGGATTTTTATTGGTGCGGCTAT
ATGTCACCTATTTTGTATTGCGTCTACTTAGCCAATAAGAAAAAAACGC
AATGGCACAGCCACTGATGACTGGTGCTATGATGTGAACGAAAATAAGC
ATCACCTTATACACCTCCTCTCTGCGGTACCCTGCAG
PstI

3. The SmaI DNA sequence between restriction sites PstI and EcoRI. Sequence was synthesized by DNA 2.0(Atum) and ligated into a vector that can be transformed into E. coli cells for replication. SmaI gene was restriction cut at PstI and EcoRI sites and isolated by gel electrophoresis. Full sequence between restriction sites with start and stop sites italicized.

| SEQ ID NO: 124 | |
|---|---|
| | C TGCAG [symbol] AG |
| 5751 | CAGGGATGAC CAACTCTTTA CACTTTGGGG AAAGCTTAAC GATCGTCAGA |
| 5801 | AGGATAATTT TCTAAAATGG ATGAAAGCTT TTGATGTAGA GAAAACTTAC |
| 5851 | CAAAAAACAA GTGGGGATAT TTTCAATGAT GATTTTTTCG ATATATTTGG |
| 5901 | TGATAGATTA ATTACTCATC ATTTCAGTAG CACGCAAGCT TTAACAAAAA |
| 5951 | CTTTATTCGA ACATGCTTTT AATGACTCCT TAAATGAATC TGGAGTTATA |
| 6001 | TCCTCTCTTG CGGAAAGTAG AACAAACCCT GGGCATGACA TAACAATCGA |
| 6051 | TAGCATAAAG GTTGCTTTAA AAACAGAAGC AGCTAAAAAT ATTAGCAAAT |
| 6101 | CATATATTCA TGTAAGTAAG TGGATGGAGT TAGGCAAGGG GGAGTGGATT |
| 6151 | CTAGAATTAT TATTAGAACG GTTTTTAGAG CATCTAGAGA ATTATGAACG |
| 6201 | TATTTTCACA CTCAGATATT TTAAAATATC CGAGTATAAA TTTAGCTACC |
| 6251 | AGCTTGTAGA AATACCCAAG AGTCTTTTGT TGGAAGCAAA AAATGCGAAA |
| 6301 | TTAGAAATAA TGTCGGGAAG CAAACAAAGC CCTAAGCCCG GCTATGGATA |
| 6351 | TGTGTTAGAT GAAAATGAAA ATAAGAAGTT TTCTCTATAC TTTGATGGTG |
| 6401 | GTGCCGAGAG AAAACTTCAA ATAAAACATT TAAATTTAGA ACATTGCATT |
| 6451 | GTTCATGGAG TTTGGGATTT TATTCTACCG CCGCCT [symbol] AATTC |

4. The rsaE DNA sequence between restriction sites PstI and EcoRI. Sequence was synthesized by DNA 2.0(Atum) and ligated into a vector that can be transformed into E. coli cells for replication. RsaE small regulatory RNA (sRNA) was restriction cut at PstI and EcoRI sites and isolated by gel electrophoresis. This contains a 5′ run-in and the mature RNA is processed out starting at the bold GAAATTAA and ending at the stretch of Is after the ACG.

| SEQ ID NO: 125 | |
|---|---|
| | CTGCAGAT GGTAGAGATA GCATGTTATA |
| 6101 | TTATGAACAT GAAATTAATC ACATAACAAA CATACCCCTT TGTTTGAAGT |
| 6151 | GAAAAATTTC TCCCATCCCC TTTGTTTAGC GTCGTGTATT CAGACACGAC |
| 6201 | GTTTTTTTGA ATTC |

5. A variant can be used for RsaE sRNA which may express the sRNA more highly which may work more effectively. This variant would start with the GAAATTAA at the 5′ end.

| SEQ ID NO: 126 | |
|---|---|
| 6110 | GAAATTAATC ACATAACAAA CATACCCCTT TGTTTGAAGT |
| 6131 | GAAAAATTTC TCCCATCCCC TTTGTTTAGC GTCGTGTATT CAGACACGAC |
| 6201 | GTTTTTTTGA ATTC |

6. The relF (E. coli) DNA sequence. This potential kill gene will be tested and cloned.

SEQ ID NO: 127
ATGAAGCAGC AAAAGGCGAT GTTAATCGCC CTGATCGTCA
TCTGTTTAAC CGTCATAGTG
ACGGCACTGG TAACGAGGAA AGACCTCTGC GAGGTACGAA
TCCGAACCGG CCAGACGGAG
GTCGCTGTCT TCACAGCTTA CGAACCTGAG GAGTAA

7. The KpnI (restriction enzyme from K. pneumoniae) DNA sequence will be tested and cloned.

SEQ ID NO: 128
atggatgtctttgataaagtttatagtgatgataataatagttatgacc
aaaaaactgtaagtcagcgtattgaagccctatttcttaataaccttgg
caaagttgtaactcgtcagcaaatcattagggcggcaactgatccaaaa
acagggaaacaaccagaaaattggcatcagagactttcagaactacgaa
ctgataaaggatatactattttatcctggcgggatatgaaggttttagc
tccgcaagagtatataatgccacacgcaacaagacgcccaaaggcagca
aagcgtgtattaccgacaaaagaaacctgggaacaggttttggatagag
ctaattactcttgcgagtggcaggaagatggtcaacactgtgggttagt
tgaaggtgatattgatcctatagggggaggcacggtcaaactaacacca
gaccatatgacacctcattcaatagatcccgcaactgatgtaaatgatc
ctaaaatgtggcaagcattgtgtggacgtcatcaagttatgaaaaaaaa
ttattgggattcaaataatgggaaaataaatgtcattggtatattgcag
tcagtaaatgagaaacaaaagaatgatgctttagagtttcttttgaatt
attatggattgaaaagataa

A synthetic Staphylococcus aureus 502a is provided herein comprising at least one molecular modification (kill switch) comprising a first cell death gene operably linked to a first regulatory region comprising a first promoter, optionally wherein the first cell death gene comprises a nucleotide sequence selected from SEQ ID NO: 122, 124, 125, 126, 127, 128, 274, 275, 284, 286, 288, 290, 315, and 317, or a substantially identical nucleotide sequence

Although kill switches (KSs) have been described for other purposes, the present KS has the unique features: i) it responds to being exposed to blood or serum; ii) it is endogenously regulated, meaning that the addition or removal of small molecules is not needed to activate or tune the KS (not an auxotroph); and iii) useful combinations of control region/toxin, and of multiple such cassettes may be used to achieve superior performance.

Expression Clamp

A synthetic microorganism is provided which comprises kill switch molecular modification comprising (i) a cell death gene operatively associated with (ii) a first regulatory region comprising a first inducible promoter which is induced by exposure to blood or serum. In order for the synthetic microorganism to durably occupy a dermal or mucosal niche in the subject, the kill switch preferably should be silent (not expressed) in the absence of blood or serum.

In order to avoid “leaky expression” of the cell death gene, the synthetic microorganism may further comprise at least a second molecular modification (expression clamp) comprising (iii) an antitoxin gene specific for the cell death gene, wherein the antitoxin gene is operably associated with (iv) a second regulatory region comprising a second promoter which is active (e.g., constitutive) upon dermal or mucosal colonization or in a media (e.g., TSB), and preferably is downregulated by exposure to blood, serum or plasma.

The basal level of gene expression (the expression observed when cells are not exposed to blood or serum, e.g., in TSB (tryptic soy broth)) in the KS strain should ideally be very low because producing the toxin prior to contact with serum would kill or weaken the strain prematurely. Even moderate cell health impairment is unacceptable because: 1) escape mutations in the KS would accumulate (KS instability) —a known phenomenon that must be avoided, and/or; 2) the natural efficacy observed with our strain in preliminary trials could be reduced or lost. To understand if leaky expression is a problem, both the absolute level of baseline expression and the fold change in serum are being measured and closely considered in the selection of the optimal control region to drive the KS.

Awareness of leaky expression does not fix the problem and the reality is that even widely used “tightly controlled” rheostatic promoters such as P_{CUP1} and P_{Gal7}, and P_{Tet}-on/off variants produce measurable mRNA transcription in the absence of specific induction. In some embodiments, an “expression clamp” is employed in which the KS cassette contains not only the serum-responsive control region that drives toxin expression, but also encodes a “translation blocking” RNA under control of a Staphylococcus aureus promoter (P_{clfB} etc) that is normally strongly active in Staphylococcus aureus during colonization of the skin, and in downregulated in blood.

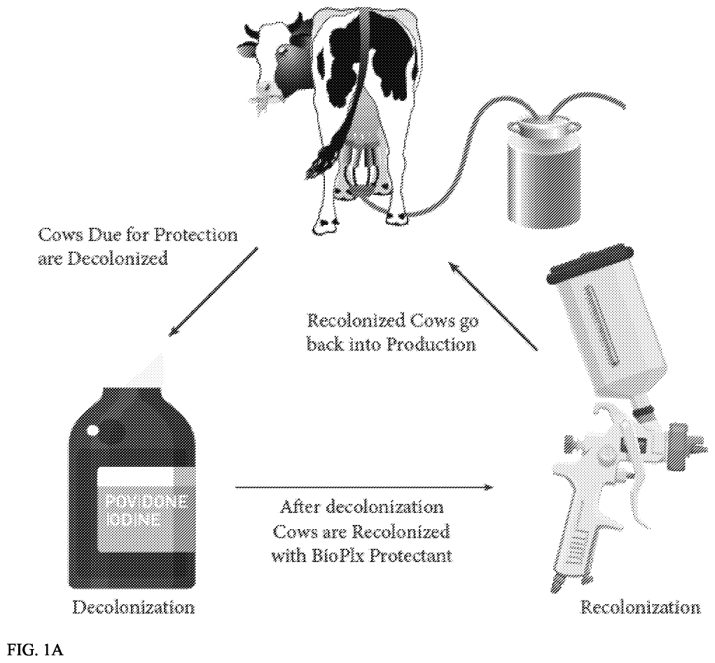

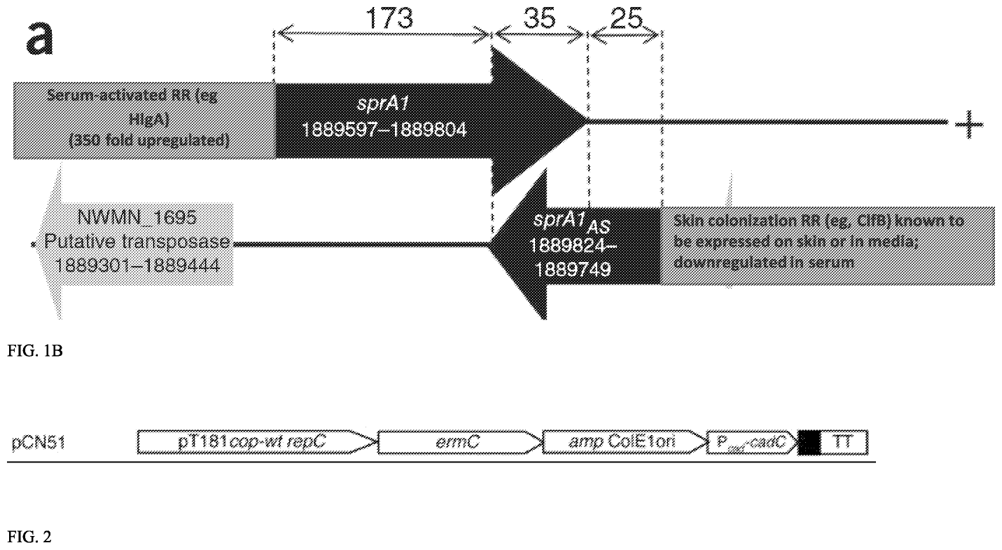

The clfB gene promoter (P_{clfB}) will be cloned to drive expression of the sprA1sprA1_{AS} RNA and the cassette will be incorporated into the same expression module as is used for expression of the sprA1 toxin from a serum-responsive promoter (eg, P_{isdB}, P_{hlgA} etc). In this strain, serum/blood exposure activates the toxin (e.g., up to 350-fold or more) but not the antitoxin, and growth in TSB or on the skin activates antitoxin but not toxin. A representative diagram of an exemplary molecular modification of a synthetic strain is shown in FIG. 1.

An Alternate Approach to a Synthetic Microorganism: KO Method

An alternative way to create a kill-switch-like phenotype in the synthetic microorganism is to disrupt (“knock-out”) one or more genes that are required for survival in blood and/or for infection of organs but that are not required (or important) for growth in media or on the skin. In some embodiments, one or more, or two or more, of the 6 genes shown in Table 5 may be employed in the KO method.

TABLE 5
Candidates for gene knockout to create an attenuated strain:

| | | Genes required for | |
|---|---|---|---|
| | Type of | survival in blood or | |
| Reference | mutagenesis | infection of organs | Reported gene function |
| Benton et al (2004) Large- | Transposon | PycA; AspB; GabP. | PycA: Pyruvate |
| Scale Identification of Genes | insertion | Mutation of these causes | carboxylase |
| Required for Full Virulence | | up to 1000-fold | AspB: Aspartate |
| of Staphylococcus aureus. J. | | reduction in rate of | aminotransferase |
| bact. 186(24): 8478-8489. | | organ infection in vivo | GabP: Gamma- |
| DOI | | | aminobutyrate |
| 10.1128/JB.186.24.8478- | | | permease |
| 8489.2004 | | | |
| Valentino et al (2014). | Transposon | Genes essential for in | SAOUHSC_01216: |
| Genes Contributing to | insertion | vitro survival in blood | succinyl CoA- |
| Staphylococcus aureus | | but not needed for | synthetase subunit b. |
| Fitness in Abscess- and | | growth in BHI liquid or | SAOUHSC_00686: |
| Infection-Related Ecologies. | | agar: | Unknown hypothetical |
| mBio5(5): e01729-14.doi: | | SAOUHSC_01216 | protein |
| 10.1128/mBio.01729-14. | | SAOUHSC_00686 | SAOUHSC_00378: |
| | | SAOUHSC_00378 | Unknown hypothetical |
| | | | protein |

In one embodiment, a synthetic microorganism is provided comprising replacement of one or more of the genes in Table 5 with unmodified or expression-clamped KS, using allelic exchange. This may further enhance the death rate of the synthetic microorganism in blood. Alternatively, the need to integrate two KSs is diminished by having one KG and one KS. In a further embodiment, a synthetic microorganism may comprise a combination of more than one KG that may have synergistic effects.

Kill Switch Regulatory Region

A synthetic microorganism comprising a kill switch is provided. The kill switch comprises a cell death gene operably linked to a regulatory region (RR) comprising an inducible promoter, as described herein.

Development of a synthetic microorganism involves identification and characterization of optimal regulatory regions (RRs) in order to drive kill switch genes; a list of serum responsive loci are chosen; RRs are identified; and Serum activation response is verified, and basal expression is investigated.

Identification and Characterization of Optimal Regulatory Regions to Drive Kill Switch Candidates.

This important phase of KS strain construction involves identifying genes that are strongly upregulated in response to human serum and/or whole heparinized blood. Once the genes are identified, their RRs, which contain the promoter and other upstream elements, are identified and annotated. In one approach, any known serum- and blood-responsive gene in Staphylococcus aureus may be employed that is known in the literature.

A RR includes the upstream regulatory sequences needed for activation (or repression) of mRNA transcription in response to stimuli. The motifs include “up” elements, −35, and −10 consensus elements, ribosome binding sites (“shine-dalgarno sequence”) and “operator” sequences which bind protein factors that strongly influence transcription. In practice for eubacteria, harnessing a 200 bp region of DNA sequence upstream of the start codon is usually adequate to capture all of these elements. However, it is preferred to deliberately identify these sequences to ensure their inclusion.

Six Staphylococcus aureus genes that are strongly upregulated by exposure to human blood or serum are shown in Table 6.

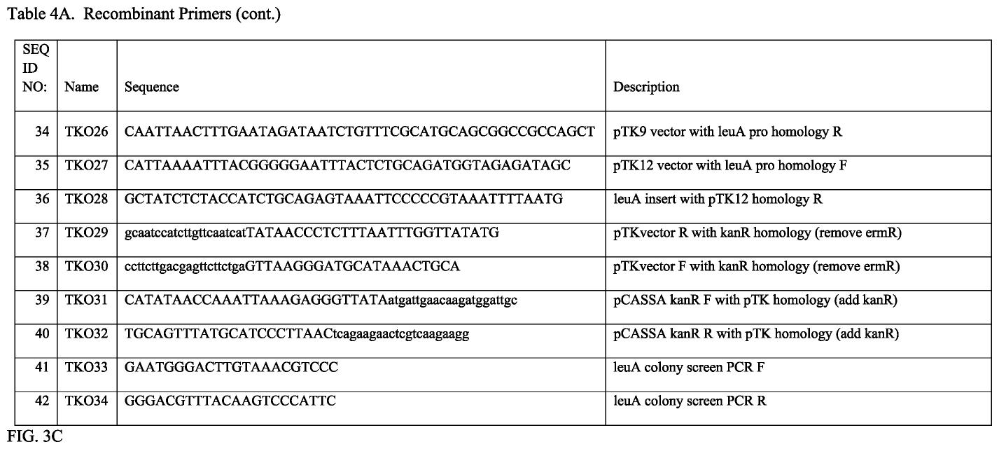

TABLE 6
Identification of candidate RRs and serum or blood inducible promoters
to drive kill switch components for driving the toxin.

| | | | | Time of | | |
|---|---|---|---|---|---|---|
| | | | | exposure | SA strain | |
| | | First author, | Fold change in | to blood | used in | |
| Gene | Function | year | serum or blood | or serum | study | Comments |
| spa | Staphyloccocal | Malachowa | ~45 fold | 90 min | USA300 | Wang 2004 |
| | Protein A; | 2011 | | | and | predicts the |
| | Ig binding; | | | | mu50 | monocistronic |
| | monocistronic | | | | | gene structure. |
| | gene | | | | | Both experi- |
| | | | | | | mental& |
| | | | | | | computational |
| | | | | | | evidence of this |
| | | | | | | structure exist |
| sir | Sir ABC; | Malachowa | 81 fold in | 30 to 120 | USA300 | High induction |
| | iron | 2011 and | serum; | minutes | and | at earliest |
| | transport | Wang 2004 | 68-fold in | | mu50 | timepoint. |
| | | | blood | | | Experimental |
| | | | (sirA; first | | | and predicted |
| | | | ORF in | | | operon |
| | | | operon) | | | structure match |
| sst | SstABCD | Malachowa | 25-fold in | 30 to 120 | USA300 | High induction |
| | operon; | 2011 and | serum; | minute | and | at earliest |
| | Iron | Wang 2004 | 15 fold in | | mu50 | timepoint. |
| | transport | | blood; | | | Experimental |
| | | | | | | and predicted |
| | | | | | | operon |
| | | | | | | structure match |
| Gamma | rbc lysis | Malachowa | ~350-fold | 90 min | USA300 | Operon |
| hemolysin | | 2011; | (FIG. 4b) | | | structure |
| hlgA | | | | | | characterized |
| | | | | | | by Cooney |
| | | | | | | 1993 |
| sai-1 | 29 kd cell | Wiltshire | 50-fold in | 16 h | 8325-4 | Serum agar and |
| (seg 7 | surface | 2001 | serum; | (O/N | | solution phase |
| surface | protein; | | 24-fold in | plating | | assays, separate |
| protein). | heme | | blood. IsdB | assay) | | pubs. Serum |
| Also | transporter; | | from the same | | | was sufficient |
| called | | | operon is | | | for induction in |
| isdA | | | upregulated240- | | | Wiltshire 2001 |
| | | | fold in serum | | | & Malachowa |
| | | | and 140-fold in | | | 2011. |
| | | | blood | | | |
| leuA | 2- | Malachowa | −6 fold | 30 to 120 | USA300 | Attractive b/c |
| | isopropyl | 2011 | downreg. in | min | | of downreg. in |
| | malate | | TSB; 15 fold | | | TSB but the |
| | synthase | | upreg in serum; | | | fold upreg. in |
| | | | 12 fold upreg | | | serum might be |
| | | | in blood | | | insufficient |
| SAUSA300_0119 | Ornithine | Malachowa | 50 fold upreg. | 30 to 120 | USA300 | Different |
| | cyclo- | 2011 | in serum, | min | | category of |
| | deaminase | | 27 fold in | | | gene than |
| | family | | blood; | | | above and also |
| | protein | | no upreg in | | | seemingly |
| | | | TSB compared | | | tightly |
| | | | to time 0 in | | | regulated in |
| | | | TSB | | | TSB |
| IrgA | Murein | Malachowa | −3.3 fold | 30 to 120 | USA300 | Attractive b/c |
| | hydrolase | 2011 | downreg in | min | | it is down- |
| | transporter | | TSB; | | | regulated in |
| | | | 12 fold upreg | | | TSB |
| | | | in serum; | | | |
| | | | 17 fold upreg | | | |
| | | | in blood | | | |
| bioA | Adenosyl | Malachowa | 107 fold upreg | 30 to 120 | USA300 | Attractive b/c |
| | methionine-8- | 2011 | in serum; | min | | very strong |
| | amino-7- | | 56 fold upreg | | | upreg and a |
| | oxononanoate | | in blood; no | | | lesser known |
| | aminotrans- | | reg in TSB | | | metabolic gene |
| | ferase | | | | | |

The full genes in each operon and the flanking sequences from strain BioPlx-01 are obtained from Genbank and annotated based on the literature plus known motif-identifying algorithms. Transcription terminators have been identified through a combination of published experiments and predictive tools.

Additional Literature evidence of expression of serum responsive promoters in TSB (or similar media) was investigated. For example, spa gene and isdA gene are disclosed in Ythier et al 2012, Molecular & Cellular Proteomics, 11:1123-1139, 2012. The sirA gene is disclosed in Dale et al, 2004 J Bacteriol 186(24) 8356-8362. The sst gene is disclosed in Morrissey et al. 2000. The hlgA gene is disclosed in Flack et al 2014, PNAS E2037-E2045. www.pnas.org/cgi/doi/10.1073/pnas.1322125111. The leuA gene is disclosed in Lei et al 2015, Virulence 6:1, 75-84.

Since these data come from many different strains and experimental systems, the entire collection may be assessed for expression in a single standardized assay system with quantitative gene expression measurements made by using real time PCR. Importantly, the basal “leaky” level of gene expression (the expression observed when cells are not exposed to blood or serum, e.g., in TSB) should be very low because producing the toxin prior to contact with serum would kill/weaken the BioPlx-XX strain (synthetic microorganism comprising a kill switch) prematurely. Even moderate cell health impairment is unacceptable because: 1) escape mutations in the KS would accumulate (KS instability) —a known phenomenon that must be avoided, and/or 2) the natural efficacy observed with BioPlx-01 could be reduced or lost. Thus, both the absolute level of baseline expression and the fold change in serum may be measured and closely considered in the selection of the optimal RRs to drive the KS. It is noted that leuA is downregulated in TSB (6-fold) and upregulated in serum (15-fold) making its RR particularly interesting candidate to control KS expression.

In some embodiments, the synthetic microorganism having a kill switch may further comprise an “expression clamp” in which the KS cassette contains not only the serum-responsive RR that drives toxin expression, but also encodes a “translation blocking” RNA antitoxin under control of a promoter that is normally active on the skin or nasal mucosa during colonization. The kill switch may encode an antitoxin that is capable of suppressing the negative effects of the cell death toxin gene.

In some embodiments, the synthetic microorganism is a Staphylococcus aureus having a molecular modification comprising a kill switch which further comprises an “expression clamp” in which the KS cassette contains not only the serum-responsive RR that drives toxin expression, but also encodes a “translation blocking” RNA antitoxin under control of a Staphylococcus aureus promoter (P_{clfB} etc.) that is normally active on the skin during colonization, for example, as shown in Table 7.

From those promoters listed on Table 6 plus real time PCR data, two or more RRs with the best mix of low basal expression and high response to serum/blood may be selected to drive KS expression. These RRs may be paired with 3 different KS genes as described herein, generating a panel of KS candidate strains for testing. The panel will include an “expression clamp” candidate as described next.

Expression Clamp to Block Toxin Expression when the KS Strain is on the Skin or Nasal Epithelia

The synthetic microorganism may comprise an expression clamp. Genes involved in Staphylococcus aureus colonization of human nares are shown in Table 7 may be employed as a second promoter for use in an expression clamp further comprising an antitoxin gene to block leaky toxin expression when the synthetic strain is colonized on skin or mucosal environments. The second promoter may be a constitutive promoter, such as a housekeeping gene. The second promote or ay be preferably downregulated in the presence of blood or serum.

TABLE 7
Genes involved in Staphylococcus aureus colonization of human nares

| Gene | Known or Putative role | Reference | Comments |
|---|---|---|---|
| clfB (Clumping factor B) | Adhesion | Wertheim H F, Walsh | 10 fold higher than |
| (ClfB) | | 2008; also Burian | Gyr in vivo; same |
| | | 2010 | high expression as |
| | | | gyr in vitro. Also, |
| | | | expression in rodent |
| | | | models and in |
| | | | humans is important |
| | | | for nasal |
| | | | colonization. It is |
| | | | expressed in exponential |
| | | | phase in vitro. Gene is |
| | | | downregulated 3-fold |
| | | | in human serum |
| | | | (Malachowa 2011) |
| autolysin (sceD) | Lytic | Stapleton M R, | expressed in exponential |
| (exoprotein D) | transglycosylase | Horsburgh M J 2007 | phase in vitro |
| walKR | essential master | Burian 2010 | In vivo expression at |
| (virulence regulator) | regulator of virulence | | time zero and at year |
| | | | 1 is on par with gyrA |
| atlA | major autolysin; | Burian 2010 | Similar characteristics |
| (Major autolysin) | Bifunctional | | as walKR but expression |
| | peptidoglycan | | is higher |
| | hydrolase | | (5 fold above gyr) |
| oatA | O-acetylation of | Burian 2010 | Similar to WalKR |
| (O-acetyltransferase A) | peptidoglycan; | | |
| | renders | | |
| | Staphylococcus aureus | | |
| | cells resistant to | | |
| | lysozyme | | |

In some embodiment, a synthetic microorganism is provided having a molecular modification comprising a kill switch and further comprising an expression clamp comprising an antitoxin gene driven by a second promoter that is normally active on the skin or nasal mucosa during colonization, optionally wherein the second promoter is selected from a gene selected from or derived from clumping factor B (clfB), autolysin (sceD; exoprotein D), walKR (virulence regulator), atlA (Major autolysin), and oatA (O-acetyltransferase A), as shown in Table 7. The constitutive second promoter may alternatively be selected from or derived from a housekeeping gene, for example, gyrB, sigB, or rho, optionally wherein the second promoter comprises a nucleotide sequence of SEQ ID NO: 324, 325, or 326, respectively, or a substantially identical sequence.

The second promoter for use in the expression clamp may be selected from a gene identified in the target microorganism that has been recognized as being downregulated upon exposure to blood or serum.

The second promoter for use in an expression clamp molecular modification should be a constitutive promoter that is preferably downregulated upon exposure to blood or serum after a period of time, e.g., after 15 minutes, 30 minutes, 45 minutes, 90 minutes, 120 minutes, 180 minutes, 240 minutes, 360 minutes, or any time point in between, to decrease transcription and/or expression of the cell death gene, by at least 2-fold, 3-fold, 4-fold, 5-fold, or at least 10-fold, compared to transcription and/or expression in the absence of blood or serum.

The second promoter may be selected by a process comprising selecting a target microorganism, selecting one or more second promoter candidate genes in the target microorganism, growing the microorganism in a media, obtaining samples of the microorganism at t=0 min, adding serum or blood to the media, obtaining samples at t=n minutes, where n=1-240 min or more, 15-180 min, or 30-120 min, performing RNA sequencing of the samples, and comparing RNA sequencing read numbers for candidate first promoter in samples obtained at obtained at t=0 min, and t=n minutes after exposure to blood or serum for the candidate first promoter gene. Alternatively, samples obtained after t=n minutes after exposure to blood or serum may be compared to t=n minutes in media without blood or serum for the candidate second promoter. Candidate second promoters may be selected from those that exhibit downregulation by RNA sequencing after target cell growth at t=n min in blood or serum, when compared to the candidate promoter in the target cell at t=0, or when compared to the candidate promoter in the target cell at t=n in media without serum or blood.

The second promoter may be selected from or derived from a promoter candidate gene identified herein for potential use in an expression clamp in Staphylococcus aureus 502a that were found to be downregulated by at least 2-fold after exposure to serum for 30 minutes as determined by RNA sequencing as compared to t=0 including phosphoribosylglycinamide formyltransferase gene CH52_00525 (−4.30 fold), phosphoribosylaminoimidazole synthetase gene CH52_00530 (−4.27 fold), amidophosphoribosyltransferase gene CH52_00535 (−4.13 fold), phosphoribosyl-formylglycineamidine synthase gene CH52_00540 (−4.04 fold), phosphoribosylformylglycinamidine synthase gene CH52_00545 (−3.49 fold), phosphoribosylaminoimidazole-succinocarboxamide gene CH52_00555 (−3.34 fold), trehalose permease IIC gene CH52_03480 (−3.33 fold), DeoR family transcriptional regulator gene CH52_02275 (−2.55 fold), phosphofructokinase gene CH52_02270 (−2.46 fold), and PTS fructose transporter subunit IIC gene CH52_02265 (−2.04 fold).

The second promoter may be selected from or derived from phosphoribosylglycinamide formyltransferase gene CH52_00525, trehalose permease IIC gene CH52_03480, DeoR family transcriptional regulator gene CH52_02275, phosphofructokinase gene CH52_02270, or PTS fructose transporter subunit IIC gene CH52_02265.

The second promoter may be a P_{clfB} (clumping factor B) gene; optionally wherein the second promoter comprises a nucleotide sequence of SEQ ID NO: 7, 117, 118, 129 or 130, or a substantially identical sequence.

In one specific example, one of the KS constructs (sprA1) is equipped with an expression clamp comprising an antitoxin (sprA1_{AS}) driven from the Clumping factor B (clfB) promoter. This promoter is one choice to drive the clamp because it is strongly expressed in TSB and during nasal/skin colonization (10 fold higher than the abundant housekeeping gene gyrA) (Burian 2010). This is directly relevant to manufacturing and use of the product, respectively. The Clumping factor B (clfB) promoter is also downregulated 3 fold in blood (Malachowa 2011), favoring clamp inactivity when. Complete inactivity in blood may not be needed because the serum-responsive promoters driving is so robustly activated in the blood.

The Clumping factor B (clfB) promoter is also stably expressed over at least 12 months during nasal colonization in humans and was also identified in rodent and in vitro models of colonization (Burian 2010).

In one example of an expression clamp, clfB is selected as a constitutive promoter for use in an expression clamp after confirmation of strong expression in TSB, and lower levels of expression in blood or serum (real time PCR), to determine its characteristics in target strain Staphylococcus aureus 502a. The clfB regulatory region is cloned to drive expression of the sprA1 antisense (antitoxin) RNA (see Table 3, first entry), and the cassette is incorporated into the same expression shuttle vector as is used for expression of the sprA1 toxin gene from a serum-responsive promoter. It is desirable that the serum/blood exposure may strongly activate the toxin but not the antitoxin, and TSB or skin/nasal epithelial exposure activates antitoxin but not toxin. This concept may be applied to the other KS genes in Table 3 below. An alternative possibility for using the clamp is for the restriction enzyme KpnI (toxin) approach for which the antitoxin may be an RNA aptamer that was recently developed as a potent inhibitor of this enzyme (Mondragon, 2015) as a means of imparting metabolic stability to the aptamer.

Awareness of leaky expression does not fix the problem and the reality is that even widely used “tightly controlled” rheostatic promoters such as P_{CUP1} and P_{Gal7}, and P_{Tet}-on/off variants produce measurable mRNA transcription in the absence of specific induction.

The expression clamp comprises a second promoter operably linked to an antitoxin gene. For example, the antitoxin gene is specific for the cell death toxin gene in the kill switch in order to be effective. Under normal physiological conditions, the expression clamp acts to prevent leaky expression of the cell death gene. When exposed to blood or serum, the second promoter operably linked to the antitoxin is downregulated, allowing expression of the cell death gene.

The synthetic microorganism may contain an expression clamp comprising an antitoxin gene which is specific for silencing the cell death gene. The antitoxin may be selected or derived from any antitoxin specific for the cell death gene in the kill switch molecular modification that is known in the art. The antitoxin gene may encode an antisense RNA specific for the cell death gene or an antitoxin protein specific for the cell death gene.

The antitoxin gene may be a sprA1 antitoxin gene, or sprA1(AS). The sprA1 antitoxin gene may comprise a nucleotide sequence of TATAATTGAGATAA CGAAAATAAGTATTTACTTATACACCAATCCCCTCACTATTTGCGGTAGTGA GGGGATTT (SEQ ID NO: 311), or a substantially identical sequence, or CCCCTCACTA CCGCAAATAGTGAGGGGATTGGTGTATAAGTAAATACTTATTTTCGTTGT (SEQ ID NO: 273), or a substantially identical sequence.

The antitoxin gene may be a sprA2 antitoxin, or sprA2(AS), and may comprise a nucleotide sequence of TATAATTAATTACATAATAAATTGAACATCTAAATACA CCAAATCCCCTCACTACTGCCATAGTGAGGGGATTTATT (SEQ ID NO: 306), or a substantially identical sequence; or TATAATTAATTACATAATAAATTGAACATCTAAAT ACACCAAATCCCCTCACTACTGCCATAGTGAGGGGATTTATTTAGGTGTTGG TTA (SEQ ID NO: 312), or a substantially identical sequence.

The antitoxin gene may be a sprG antitoxin gene, also known as sprF, and may comprise a nucleotide sequence of (5′-3′) ATATATAGAAAAAGGG CAACATGCGCAAACATGTTACCCTAATGAG CCCGTTAAAAAGACGGTGGCTATTTTAGATTAAAGATTAAATTAATAACCA TTTAACCATCGAAACCAGCCAAAGTTAGCGATGGTTATTTTTT (SEQ ID NO: 307), or a substantially identical sequence. Pinel-Marie, Marie-Laure, Régine Brielle, and Brice Felden. “Dual toxic-peptide-coding Staphylococcus aureus RNA under antisense regulation targets host cells and bacterial rivals unequally.” Cell reports 7.2 (2014): 424-435.

The antitoxin gene may be a yefM antitoxin gene which is specific for silencing yoeB toxin gene. The yefM antitoxin gene may comprise a nucleotide sequence of MIITSPTEARKDFYQLLKNVNNNHEPIYISGNNAENNAVIIGLEDWKSIQETIYLE STGTMDKVREREKDNSGTTNIDDIDWDNL (SEQ ID NO: 314), or a substantially identical nucleotide.

The antitoxin gene may be a lysostaphin antitoxin gene specific for a lysostaphin toxin gene. The lysostaphin antitoxin may comprise a nucleotide sequence of TATAATTGAGATATGTTCATGTGTTATTTACTTATACACCAATCCCCTCACT ATTTGCGGTAGTGAGGGGATTTTT (SEQ ID NO: 319), or a substantially identical nucleotide sequence.

The antitoxin gene may be a mazE antitoxin gene that targets mazF. The mazE toxin gene may comprise a nucleotide sequence of ATGTTATCTTTTAGTCAAAAT AGAAGTCATAGCTTAGAACAATCTTTAAAAGAAGGATATTCACAAATGGCT GATTTAAATCTCTCCCTAGCGAACGAAGCTTTTCCGATAGAGTGTGAAGCA TGCGATTGCAACGAAACATATTTATCTTCTAATTC (SEQ ID NO: 322), or a substantially identical sequence.

The antitoxin gene may alternatively be designed as follows. In Staphylococcus aureus, there are two main methods used for gene silencing. In one style of gene silencing, which is exemplified by sprA1, antisense RNA binds to the 5′ UTR of the targeted gene, blocking translation of the gene and causing premature mRNA degradation. Another style of gene silencing is used for genes that do not have a transcriptional terminator located close to the stop codon. Translation can be controlled for these genes by an antisense RNA that is complementary (˜3-10 bases) to the 3′ end of the targeted gene. The antisense RNA will bind to the mRNA transcript covering the sequence coding for the last couple codons and creating double stranded RNA which is then targeted for degradation by RNaseIII.

Since there are many examples of RNA silencing in Staphylococcus aureus that have been identified with demonstrated ability to control their target genes, these regions and sequences may be used as a base for designing the toxin/antitoxin cassettes. This approach requires only small changes in the DNA sequences.

In the present disclosure, the antitoxin for a cell death gene may be designed to involve antisense binding to 5′UTR of targeted gene. The toxin gene may be inserted into the PepA1 reading frame, and the 12 bp in the endogenous sprA1 antisense is swapped out for a sequence homologous to 12 bp towards the beginning of the heterologous toxin gene.

In one example, Holin inserted into the sprA1 location can be controlled by the antisense RNA fragment encoded by (12 bp Holin targeting sequence in BOLD)=TATA ATTGAGAT AGTTTCATTAGCTATTTACTTATACACCAATCCCCTCA CTATTT GCGGTAGTGA GGGGATTTTT (SEQ ID NO: 308).

In another example, 187-lysK inserted into the sprA1 location can be controlled by the antisense RNA fragment encoded by (12 bp 187-lysK targeting sequence in BOLD) TATAATTGAGAT TTTAGGCAGTGC TATTTACTTATACACCAA TCCCCTCA CTATTTGCGGT AGTGAGGGGATTTTT (SEQ ID NO: 309).

The antitoxin specific for the cell death gene may involve antisense binding to the 3′ UTR of the toxin gene. This method involves inserting the heterologous toxin in the place of sprG in the genome of Staphylococcus aureus, and adding an additional lysine codon (AAA) before the final stop codon. The last 6 bases of the coding region (AAAAAA) plus the stop codon (TAA) overlap with the 3′ region of the endogenous sprF antitoxin. When the sprF RNA is transcribed at a rate of 2.5 times greater than the heterologous toxin gene, it will form a duplex with the 3′UTR region of the toxin transcript, initiating degradation by RNaseIII and blocking the formation of a functional peptide. Since the 3′ end of both of the heterologous toxins were manipulated in the same manner to overlap with the sprF sequence (adding the codon AAA in front of the TAA stop codon), which is also the same as the endogenous sprG 3′ end, the sequence of the antitoxin will remain the same for all three of these toxin genes. For example, the sprG antitoxin gene (sprF) may comprise the nucleotide sequence ATATATAGAAAAA GGGCAACATGCGCAAACATGTTACCCTAATGAGCCC GTTAAAAAGACGGTGGCTATTTTAGATTAAAGATTAAATTAATAACCATTT AACCATCGAAACCAGCCAAAGTTAGCGATGGTTATTTTTT (SEQ ID NO: 310).

The antitoxin gene may comprise a nucleotide sequence selected from any of SEQ ID NOs: 273, 306, 307, 308, 309, 310, 311, 312, 314, 319, 322, 342, 347, 362, 364, 368, 373, 374, 375, 376, 377, and 378, or a substantially identical sequence thereof.

The antitoxin gene may or may not encode an antitoxin peptide. Wherein the synthetic microorganism is derived from a Staphylococcus aureus strain, the antitoxin peptide may be specific for the toxin peptide encoded by the cell death gene. For example, when the toxin gene is a yoeB toxin gene, e.g., encoding a toxin peptide comprising an amino acid sequence of SEQ ID NO: 316, the antitoxin gene may encode a yefM antitoxin protein comprising the amino acid sequence of MIITSPTEARKDFYQLLKNVNNNHEPI YISGNNAENNA VIIGLEDWKSIQETIYLESTGTMDKVREREKDNSGTTNIDDIDWDNL (SEQ ID NO: 314), or a substantially similar sequence. As another example, wherein the antitoxin gene is a mazF toxin gene, e.g., encoding a toxin peptide comprising an amino acid sequence of SEQ ID NO: 321, the antitoxin gene may be an mazE antitoxin gene, e.g., encoding an antitoxin protein comprising an amino acid sequence of MLSFSQNRSHSLEQSLKEGYSQ MADLNLSLANEAFPIECEACDCNETYLSSNSTNE (SEQ ID NO: 323), or a substantially similar sequence.

Three KS candidate genes were selected as being of particular interest because they elicit cell death in 3 disparate ways. In some embodiments, the synthetic microorganism comprises one or more, two or more or each of sprA1, kpnI or rsaE to achieve maximal death rates as early data instruct. The sprA1 mechanism of action is a loss of plasma membrane integrity/function by expression of a pore-forming peptide. the kpnI mechanism of action involves destruction of the Staphylococcus aureus genome with a restriction enzyme. The rsaE mechanism of action involves impairment of central metabolism including TCA cycle and tetrahydrofolate biosynthesis.

In some embodiments, the synthetic microorganism comprises regulatory region comprising a first promoter operably linked to a cell death gene, wherein the cell death gene encodes a toxin peptide or protein, and wherein the first promoter is upregulated upon exposure to blood or serum. The cell death gene may be a sprA1 gene. SprA1 encodes toxin peptide PepA1 as described in Sayed et al., 2012 JBC VOL. 287, NO. 52, pp. 43454-43463, Dec. 21, 2012. PepA1 induces cell death by membrane permeabilization. PepA1 has amino acid sequence: MLIFVHIIAPVISGCAIAFFSYWLSRRNTK (SEQ ID NO: 104). Related antimicrobial peptides include MMLIFVHIIAPVISGCAIAFFSYWLSRRNTK (SEQ ID NO: 105), AIAFFSYWLSRRNTK (SEQ ID NO: 106), IAFFSYWLSRRNTK (SEQ ID NO: 107), AFFSYWLSRRNTK (SEQ ID NO: 108), FFSYWLSRRNTK (SEQ ID NO: 109), FSYWLSRRNTK (SEQ ID NO: 110), SYWLSRRNTK (SEQ ID NO: 111), or YWLSRRNTK (SEQ ID NO: 112), as described in WO 2013/050590, which is incorporated herein by reference. The cell death gene may be an sprA2 gene. The sprA2 gene may encode a toxin MFNLLINIMTSALSGCLVAFFAHWLRTRNNKKGDK (SEQ ID NO: 305). The cell death gene may be a Staphylococcus aureus yoeB gene which may encode a yoeB toxin having the amino acid sequence of MSNYTVKIKNSAKSDLRKIKHSYLKKSFLEIVETLKND PYKITQSFEKLEPKYLERYSRRINHQHRVVYTVDDRNKEVLILSAWSHYD (SEQ ID NO: 316), or a substantially similar sequence. The cell death gene may be a Staphylococcus simulans gene which may encode a metallopeptidase toxin gene having an amino acid sequence of MTHEHSAQWLNNYKKGYGYGPYPLGINGGMHYGVDFFMNIGTPVKAISSGKI VEAGWSNYGGGNQIGLIENDGVHRQWYMHLSKYNVKVGDYVKAGQIIGWSG STGYSTAPHLHFQRMVNSFSNSTAQDPMPFLKSAGYGKAGGTVTPTPNTGWK TNKYGTLYKSESASFTPNTDIITRTTGPFRSMPQSGVLKAGQTIHYDEVMKQDG HVWVGYTGNSGQRIYLPVRTWNKSTNTLGVLWGTIK (SEQ ID NO: 318), or a substantially similar sequence. The cell death gene may be a mazF toxin gene that encodes a mazF toxin comprising an amino acid sequence of MIRRGDVYLADLSPVQGSEQGGVRPVVIIQNDTGNKYSPTVIVAAITGRINKAK IPTHVEIEKKKYKLDKDSVILLEQIRTLDKKRLKEKLTYLSDDKMKEVDNALMI SLGLNAVAHQKN (SEQ ID NO: 321), or a substantially similar sequence.

The cell death gene may encode a toxin peptide or protein comprising an amino acid sequence of SEQ ID NO: 104, 105, 106, 107, 108, 109, 110, 111, 112, 285, 287, 289, 291, 305, 316, 318, 321, 411, 423, 596, or a substantially similar amino acid sequence. Preferably, the first promoter is silent, is not active, or is minimally active, in the absence of blood or serum.

PepA1 is a toxic pore forming peptide that causes Staphylococcus aureus death by altering essential cell membrane functions. Its natural role is unknown but speculated to be altruistic assistance to the Staphylococcus aureus population/culture by killing of cells that are adversely affected by environmental conditions. By over-expressing this gene a rapid and complete cell death occurs in the presence of serum. Of note, sprA1 mRNA translation is repressed by an antisense RNA called sprA11 (SprA1 antisense). The cis-encoded SprA1_{AS} RNA operates in trans to downregulate the sprAl-encoded peptide expression in vivo, as described in WO 2013/050590, which is incorporated herein by reference. The antisense RNA may in fact be a convenient safeguard to minimize “leaky” toxicity. It will be driven from a promoter that is expressed in Staphylococcus aureus on the human skin and nasal epithelia during colonization. Advantages of sprA1 include the expression of a small peptide, having known structure and activity.

In a particular embodiment, a synthetic microorganism is provided comprising a first cell death gene sprA1 operably linked to a first regulatory region comprising a blood and/or serum inducible first promoter comprising a nucleotide sequence of any one of SEQ ID NOs: 1, 2, 3, 4, 5, 6, 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370. The first promoter may be upregulated greater than 5-fold, greater than 10-fold, greater than 50-fold, greater than 100-fold, greater than 300-fold, or greater than 600-fold after 15, 30, 45, 60, 90, 120, 180 or 240 minutes of incubation in blood or serum. The first promoter may be upregulated greater than 5-fold after 90 minutes of incubation in serum and may be selected from fhuA, fhuB, isdI, isdA, srtB, isdG, sbnE, sbnA, sbnC, and isdB. The first promoter may be upregulated greater than 100-fold after 90 minutes of incubation in serum and may be selected from isdA, srtB, isdG, sbnE, sbnA, sbnC, and isdB.

The cell death gene may encode an antimicrobial peptide comprising an amino acid sequence of SEQ ID NO: 104, 105, 106, 107, 108, 109, 110, 111, 112, 285, 287, 289, 291, 305, 316, 318, 321, 411, 423, 596, or a substantially similar amino acid sequence thereof.

The cell death gene may be selected from any known Staphylococcus spp. toxin gene. The cell death gene may be selected from a sprA1 toxin gene, sprA2 toxin gene, 187-lysK toxin gene, holin toxin gene, sprG toxin gene, yoeB toxin gene, lysostaphin toxin gene, metallopeptidase toxin gene, or mazF toxin gene, or a substantially identical toxin gene. The toxin gene may comprise a nucleotide sequence of SEQ ID NO: 274, 275, 284, 286, 288, 290, 304, 315, 317, or 320, or a substantially identical nucleotide sequence thereof.

The cell death gene may be sprA1 which encodes the antimicrobial peptide PepA1. In some embodiments, the synthetic microorganism further comprises an antitoxin gene SprA1-AS operably linked to a second regulatory region comprising a second promoter comprising a nucleotide sequence of clfB comprising a nucleotide sequence of SEQ ID NO: 7, 117, 118, 129 or 130, or a substantially identical sequence.

In some embodiments, the synthetic microorganism comprises a restriction enzyme KpnI (Klebsiella pnemoniae) gene. KpnI protects bacterial genomes against invasion by foreign DNA. High-level expression of (eg) 6-bp recognition restriction enzyme KpnI will efficiently cleave the Staphylococcus aureus genome. In some embodiments, the expression vector (below) will be engineered to lack cleavage recognition sites by (eg) adjustment of codon usage. The 6-base recognition sequence occurs once every 4096 bp, cutting the 2.8 MB genome of Staphylococcus aureus into ˜684 fragments. KpnI has the advantage of rapid activity. In some embodiments, “leaky” expression problem may be managed by expressing an RNA aptamer as the clamp as described above for sprA1.

In some embodiments, the synthetic microorganism comprises a rsaE gene. The rsaE gene is a small RNA (93 nt) that coordinately inhibits 2 different metabolic pathways by targeting translation initiation of certain housekeeping mRNAs encoding enzymes of THE biosynthesis pathway and citric acid cycle; high-level expression is toxic. By over-expressing RseE growth impairment occurs due to inhibition of essential housekeeping enzymes. This occurs by binding to the Opp3A and OppB mRNAs in the ribosome-binding site and start codon region, preventing translation. Both genes encode components of the ABC peptide transporter system and affect the supply of essential nitrogen/amino acids in the cell, impairing central biochemical metabolism directly and indirectly. Advantages include severe growth inhibition (10,000 fold over empty vector controls), and efficient multifunctionality because a single sRNA impairs expression of multiple essential biochemical pathways. Geissman et al. 2009 and Bohn et al. 2010 report on the natural function of RsaE.

Creation of a panel of serum-activated kill switch (KS) plasmid candidates for expression in Staphylococcus aureus is performed wherein serum responsive RRs are sub-cloned to Staphylococcus aureus shuttle vectors; cell death genes are inserted downstream of RRs, and sequenced; feasibility of leaky expression repressor “expression clamp” is tested; and candidate strains are completed and evaluated to select lead candidate(s) that exhibit rapid and complete death, and good baseline viability.

Chromosomal integration of optimal kill switch candidates is important for long-term stable expression. In addition, comparison of death rate extent and stability of strains in vitro is performed. Insertion of up to 3 optimal kill switch cassettes alone and in 3 combinations of two, for a total of up to 6 strains is performed. This achievement may require a multistep cloning in E. coli to build the constructs. For example, E. coli strain DC10B may be employed. DC10B is an E. coli strain that is only DCM minus (BEI product number NR-49804). This is one way to generate DNA that can be readily transfected into most Staphylococcus aureus strains. To this end, stable integrants are obtained, and plasmid vector is excised during counter selection. The rate and extent of serum-induced cell death is confirmed and characterized, and genetic stability is determined for all 6 strains. A non-human functional test of preferred KS strain candidates is performed including a functional test of strain death in vivo; and a functional test of colonization-skin discs.

In some embodiments, a method for preparing a synthetic Staphylococcus aureus strain from BioPlx-01 is provided comprising (1) producing a shuttle vector pCN51 in mid-scale in E. coli, (2) cloning cell death genes into pCN51 in E. coli under Cd-inducible promoter P_{cad}, (3) replacing P_{cad} with serum-responsive promoters and optionally inserting expression clamp, (4) verifying constructs by sequencing KS cassettes, (5) electroporating into Staphylococcus aureus RN4220 and selecting transformants on erythromycin plates (this strain is restriction minus and generates the right methylation pattern to survive in BioPlx-01), (6) preparing plasmid from RN4220 and restriction digest to confirm identification, (7) electroporating plasmids into BioPlx-01 and select on erythromycin plates, and (8) isolating strains. Stains produced in this fashion are ready for performance testing and serum experimentation. The method is further described in detail herein.

In some embodiments, a method for performance testing a synthetic Staphylococcus aureus strain from BioPlx-01 is provided comprising (1) growing in TSB plus antibiotic as selective pressure for plasmid, (2) comparing growth to WT BioPlx-01 optionally generating a growth curve, (3a) for Cd-promoter variants, washing and shifting cells to Cd-medium (control is BioPlx-01 containing empty vector with no cell death gene) —or—(3b) for KS variants, washing and shifting cells to serum (control is WT BioPlx-01 containing empty vector with no cell death gene), and (4) monitoring growth using OD_{630 nm} with plate reader, optionally for extended period with monitoring for escape mutants. For whole blood test, the method is only performed on preferred candidates and using colony forming units (CFUs) on TSA as death readout. If colonies form on kill switch bearing strains after they have been exposed to blood, the plasmid should be sequenced to check for mutations. If there are escape mutants, shuttle plasmid out to E. coli and sequence whole plasmid.

Method for Creation of Serum-Activated Kill Switch (KS) Plasmid Candidates for Expression in Staphylococcus aureus (SA)

Methods are provided for evaluation of cell death induction comprises recombinant construction of the synthetic microorganism comprising cloning the genes into an E. coli-SA shuttle vector and transfecting this vector into BioPlx-01 for evaluation.

Step 1: Request Shuttle Vector PCN51

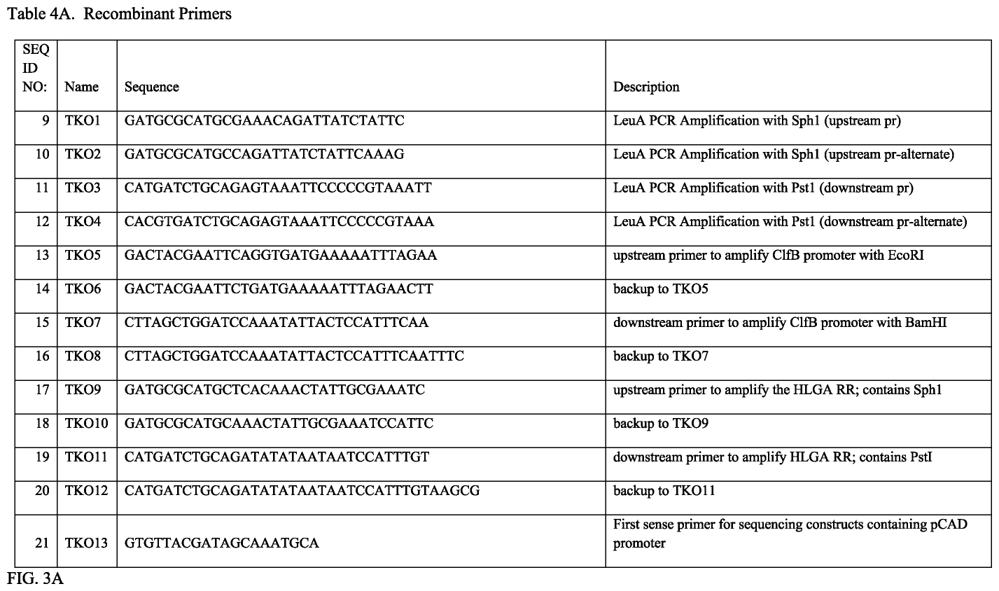

A commercially available shuttle vector is obtained such as PCN51 (available through BEI) is one excellent choice as it contains: i) a cadmium-inducible promoter that can be used in positive control strains to prove the toxins are expressed and functional; ii) a universal Transcription terminator (TT) that will apply to all of our constructs; and, iii) well-established replicons for E. coli and Staphylococcus aureus. A schematic of commercially available shuttle vector pCN51 (BEI cat #NR-46149) is shown in FIG. 2. Genetic elements shown of pCN51 shuttle plasmid are shown in Table 8.

TABLE 8
Elements of pCN51 Shuttle Vector
Shuttle Plasmid pCN51 (BEI cat # NR-46149)

| Element | Purpose |
|---|---|
| pT181cop-WT repC | SA replication machinery |
| ermC | erythromycin resistance |
| Amp | beta-lactamase; confers resistance to ampicillin in |
| | E coli |
| ColE1 Ori | Origin of replication for E coli |
| Pcad-cadC | Cadmium-inducible promoter |
| MCS (black box) | Multiple Cloning Site; unique sites for cloning our |
| | KS. |
| TT | blaZ transcription terminator |

Promoter sequences (7) used in development are shown below, the base pair numbers in leuA, hlgA and Cadmium promoters correspond to pCN51 vector location.

1. leuA promoter (P_{leuA}) sequence between restriction sites SphI and PstI (underlined) amplified from genomic BioPlx-01 (502a) DNA.

| SEQ ID NO: 114 | |
|---|---|
| SphI | |
| | GCATGCGAAA CAGATTATCT |
| 5501 | ATTCAAAGTT AATTGTAAGA AAATTTAAAA TATTTGTTGA CATACTAAAG |
| 5551 | CAGATATAGT AAATTAAATT TATCAAATTT TTAGACAATT CTAACTATTA |
| 5601 | AAGTGATATA TACCATTCAC GGAAGGAGTA TAATAAAATG CTTAATCAAT |
| 5651 | ATACTGAACA TCAACCGACA ACTTCAAATA TTATTATTTT ATTATACTCT |
| 5701 | TTAGGACTCG AACGTTAGTA AATATTTACT AAACGCTTTA AGTCCTATTT |
| 5751 | CTGTTTGAAT GGGACTTGTA AACGTCCCAA TAATATTGGG ACGTTTTTTT |
| 5801 | ATGTTTTATC TTTCAATTAC TTATTTTTAT TACTATAAAA CATGATTAAT |
| 5851 | CATTAAAATT TACGGGGGAA TTTACTCTGC AG |

PstI

2. hlgA promoter (P_{hlgA}) sequence between restriction sites SphI and PstI amplified from genomic BioPlx-01 (502a) DNA.

| SEQ ID NO: 115 | |
|---|---|
| SphI | |
| | GCATGC AAACTATTGC |
| 5501 | GAAATCCATT CCTCTTCCAC TACAAGCACC ATAATTAAAC AACAATTCAA |
| 5551 | TAGAATAAGA CTTGCAAAAC ATAGTTATGT CGCTATATAA ACGCCTGCGA |
| 5601 | CCAATAAATC TTTTAAACAT AACATAATGC AAAAACATCA TTTAACAATG |
| 5651 | CTAAAAATGT CTCTTCAATA CATGTTGATA GTAATTAACT TTTAACGAAC |
| 5701 | AGTTAATTCG AAAACGCTTA CAAATGGATT ATTATATATA TGAACTTAAA |
| 5751 | ATTAAATAGA AAGAAAGTGA TTTCTCTGCA G |

PstI

3. Cadmium promoter (P_{cad}) sequence between restriction sites SphI and PstI. This promoter is used for controls and is part of the original pCN51 vector from BEI Resources (https://www.beiresources.org/).

| SEQ ID NO: 116 | |
|---|---|
| SphI | |
| | GCATGCGCAC TTATTCAAGT |
| 5501 | GTATTTTTTA ATAAATTATT TTACTTATTG AAATGTATTA TTTTCTAATG |
| 5551 | TCATACCCTG GTCAAAACCG TTCGTTTTTG AGACTAGAAT TTTATGCCCT |
| 5601 | ACTTACTTCT TTTATTTTCA TTCAAATATT TGCTTGCATG ATGAGTCGAA |
| 5651 | AATGGTTATA ATACACTCAA ATAAATATTT GAATGAAGAT GGGATGATAA |
| 5701 | TATGAAAAAG AAAGATACTT GTGAAATTTT TTGTTATGAC GAAGAAAAGG |
| 5751 | TTAATCGAAT ACAAGGGGAT TTACAAACAG TTGATATTTC TGGTGTTAGC |
| 5801 | CAAATTTTAA AGGCTATTGC CGATGAAAAT AGAGCAAAAA TTACTTACGC |
| 5851 | TCTGTGTCAG GATGAAGAGT TGTGTGTTTG TGATATAGCA AATATCTTAG |
| 5901 | GTGTTACGAT AGCAAATGCA TCTCATCATT TACGTACGCT TTATAAGCAA |
| 5951 | GGGGTGGTCA ACTTTAGAAA AGAAGGAAAA CTAGCTTTAT ATTCTTTAGG |
| 6001 | TGATGAACAT ATCAGGCAGA TAATGATGAT CGCCCTAGCA CATAAGAAAG |
| 6051 | AAGTGAAGGT CAATGTCTGA ACCTGCAG |

PstI

4. clfB promoter (P_{clfB}) to drive the antisense regulatory RNA sprA1_{AS}. This is the forward sequence with EcoRI and BamHI sites. This sequence is put in reverse to drive the sprA1_{AS} to potentially act as a clamp to keep the sprAI gene regulated in the absence of blood. Underlined represents EcoRI and BamHI sites, respectively.

SEQ ID NO: 117
EcoRI
GAATTCAGGTGATGAAAAATTTAGAACTTCTAAGTTTTTGAAAAGTAAAAAATTTGTAATA
GTGTAAAAATAGTATATTGATTTTTGCTAGTTAACAGAAAATTTTAAGTTATATAAATAGGA
AGAAAACAAATTTTACGTAATTTTTTTCGAAAAGCAATTGATATAATTCTTATTTCATTATAC
AATTTAGACTAATCTAGAAATTGAAATGGAGTAATATTTGGATCC

P_{clfB} as it is cloned in pCN51 vector with EcoRI and BamHI reversed.

SEQ ID NO: 118
BamHI
GGATCCAAATATTACTCCATTTCAATTTCTAGATTAGTCTAAATTGTATAATGAAATAAGAA
TTATATCAATTGCTTTTCGAAAAAAATTACGTAAAATTTGTTTTCTTCCTATTTATATAACTT
AAAATTTTCTGTTAACTAGCAAAAATCAATATACTATTTTTACACTATTACAAATTTTTTACT
TTTCAAAAACTTAGAAGTTCTAAATTTTTCATCACCTGAATTC

5. The sirA promoter (P_{sirA}) as found in the NCBI 502a complete genome. This sequence was taken 300 base pairs upstream of the sirA start codon as shown underlined below.

SEQ ID NO: 119
ttagaaagatttacttttatatatgaagagactggattaaatactttta
ttgacgtaaaaattcacttttgaaccgttcaatatcttgccgattttta
tataacagctacaaataaaatataacagtttgattttacagcctcggta
aatcgtatgacaaacaaaaattttgtgctatcacaacatttgcaacgtc
ttaacaagtcatctataaacatttctaaatatttaacattacttatgcg
tcatttattgctaaaattattgtattaaaatatacatagaattgatggg
atatcATG

6. The sstA promoter (P_{sstA}) as found in the NCBI 502a complete genome. This sequence was taken 300 base pairs upstream of the sstA start codon as shown underlined below.

SEQ ID NO: 120
acgaaaaattaattaacatcgcattgtttattactgcaactattacagc
attggtagtggtgactgttggaacattaccgttcttaggactagtaata
ccaaatattatttcaatttatcgaggtgatcatttgaaaaatgctatcc
ctcatacgatgatgttaggtgccatctttgtattattttctgatatagt
tggcagaattgttgtttatccatatgaaataaatattggtttaacaata
ggtgtatttggaacaatcattttccttatcttgcttatgaaaggtagga
aaaattATG

7. The isdA promoter (P_{isdA}). This sequence was taken 300 base pairs upstream of the SstA start site as shown underlined below from the NCBI 502a complete genome.

SEQ ID NO: 121
CTATCTGCGGCATTTGCAGAATTACTGAATGTCGCGATGATGATAATTA
ACGCTAAAATCGTTGTATTAAAAACTTTTAAAATATTTTTCAAAACATA
ATCCTCCTTTTTATGATTGCTTTTAAGTCTTTAGTAAAATCATAAATAA
TAATGATTATCATTGTCAATATTTATTTTATAATCAATTTATTATTGTT
ATACGGAAATAGATGTGCTAGTATAATTGATAACCATTATCAATTGCAA
TGGTTAATCATCTCATATAACAACACATAATTTGTATCCTTAGGAGGAA
AACAACATG.

In some embodiments, a plasmid, vector, or synthetic microorganism is provided comprising a molecular modification comprising a cell death gene operably linked to an inducible blood or serum responsive first promoter comprising a nucleotide sequence selected from the group consisting of SEQ ID NO: 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical nucleotide sequence. In some embodiments, the molecular modification further comprises an expression clamp comprising an antitoxin gene operably linked to a second promoter comprising a nucleotide sequence selected from SEQ ID NO: 7, 117, 118, 129 or 130.

Step 2: Cloning Best Two Serum-Responsive RRs into the Shuttle Vector (E. coli Host)

Cloning of candidate serum-responsive RRs into the shuttle vector (E. coli host) comprises: (a) PCR amplification of the best two preferred serum-responsive RRs from BioPlx-01 genomic DNA (gDNA); and (b) replacing the Cadmium-inducible promoter with these RR fragments in pCN51 to create two new plasmids (RR1 and RR2), and (3) selecting clones in E. coli DH10B (or DH5 alpha) and sequencing of insertions.

The following KS genes are obtained from Staphylococcus aureus gDNA or by de novo synthesis: (i) sprA1/sprA1_{AS}: synthetic; (ii) RsaE: Staphylococcus aureus genomic DNA. And (iii) KpnI: synthetic. For genes amplified from gDNA, PCR primers are used with relevant restriction enzymes for cloning. For synthetic genes, the cloning sites will be included at synthesis and any undesirable sites removed during construction. For example, KpnI sites will be removed from the kpn1 cassette to prevent auto-digestion. The KS genes are inserted downstream of serum-responsive RRs in plasmids RR1 and RR2, generating all constructs listed below. Insert the KS genes downstream of Cd-inducible promoter in pCN51 to create positive control constructs. See additional relevant sequences and primer sequences provided herein useful for these steps, for example, Tables 2, 3 and 4. Sequencing of promoters and inserts of all constructs is performed to ensure that mutations have not accumulated in the construction process

A list of Plasmid constructs to be produced is shown below. All but 2, 4, 8 and 11 will be transfected into Staphylococcus aureus.

1. Cd-inducible promoter-sprA1

2. Cd-inducible promoter-reverse orientation sprA1

3. Serum responsive RR1—sprA1

4. Serum responsive RR1—reverse orientation sprA1

5. Serum responsive RR1—sprA1+P_{clfB}-sprA1_{AS}

6. Serum responsive RR2—sprA1

7. Serum responsive RR1—rsaE

8. Serum responsive RR1—rsaE-reverse orientation

9. Serum responsive RR2—rsaE

10. Serum responsive RR1—kpnI

11. Serum responsive RR1—kpnI reverse orientation

12. Serum responsive RR2—kpnI

The reverse orientation constructs are being created in the process, because if a cell death gene has some basal toxicity even in growth medium, it may not be possible to obtain the forward orientation construct. Such a negative result is not conclusive unless the reverse orientation construct is readily obtained in side-by-side fashion.

Step 3: Transfect Plasmids into Intermediate Staphylococcus aureus RN4220 (to Obtain Correct DNA Methylation Pattern).

There is no need to transfect reverse orientation constructs; but transfection of pCN51 empty vector is performed as follows:

A. Electroporate into RN4220;
B. Select transformants on plates containing erythromycin; and
C. Isolate and confirm plasmid ID with restriction digests.

Step 4: Transfect into BioPlx-01

A. Electroporate plasmids from step 3C into competent BioPlx-01;
B. Select transformants by erythromycin resistance; and
C. Isolate and confirm plasmid ID with restriction digests; save stocks of 9 strains.

Step 5: Test KS Expression and Extent and Rate of Death in Response to Serum and Blood Exposure

A. Qualitative test of expression of kill genes with real time PCR pre- and post-blood/serum exposure. This will: i) confirm the strain construction; ii) correlate onset of toxin production with onset of death, and iii) determine promoter “leakiness” in the context of the KS;
B. Cell death induction curves in serum/blood compared to TSB (killing extent and kinetics by CFU); and
C. Simple growth rate comparison of BioPlx-01 containing empty vector vs. BioPlx-01 with the KS plasmids.

Step 6: Measure the Rate of KS Mutation

Count colonies that grow on serum or blood agar plates and/or in serum containing liquid media over several hundred generations via serial passaging. Determine if mutation rate is acceptable. It has been reported that the rate of functional KS loss is 10⁻⁶ for one copy of a KS gene, but as low as 10⁻¹⁰ for two copies of the same or different KS genes from two different promoters (Knudsen 1995; reporting on actual mutation rate assay measurements).

Step 7: Analysis and Interpretation

The best KS strain(s) are those with unaffected growth rates (and colonization potential); and that show rapid and complete death in response to blood and/or serum; and that have stable molecular modifications.

Step 8: Determine Need for Inserting Multiple KS Cassettes

If the molecular stability of one KS is deemed inadequate, a second and different functional KS from the list of 9 candidates (if another functional one exists) will be added to the plasmid and a re-test of killing and stability will be performed. A dramatic improvement in KS stability is anticipated on the basis of Knudsen 1995 and theoretical calculations.

Method for Chromosomal Integration of Optimal Kill Switch(es), for Long-Term Stable Expression

The optimal serum/blood responsive KS construct(s) will be integrated into the chromosome precisely at a pre-selected location known to tolerate insertions without notably altering the cell's biology.

Step 1: Obtain an Integrative Vector for Use in Staphylococcus aureus.

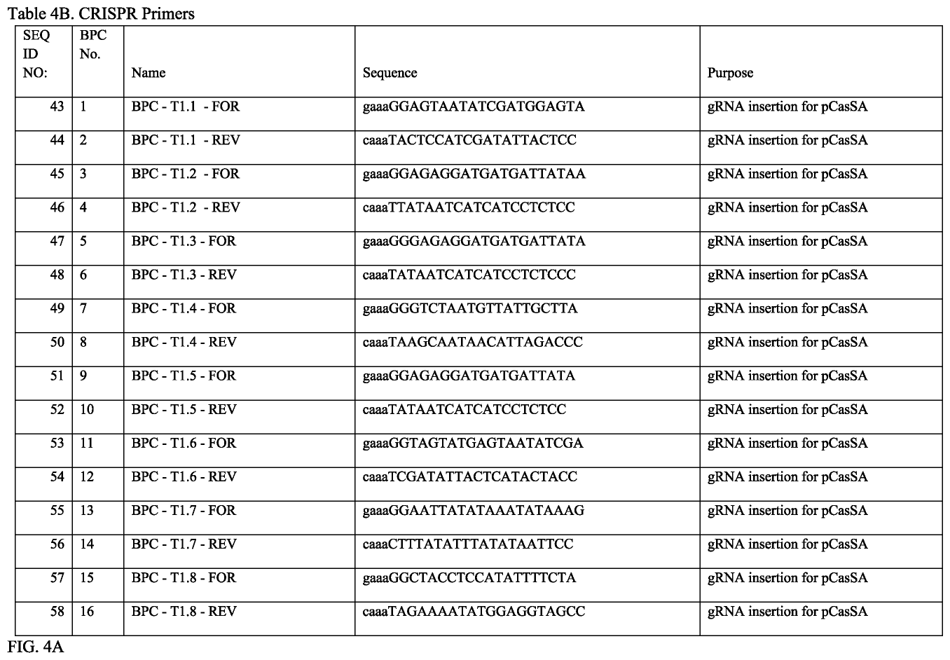

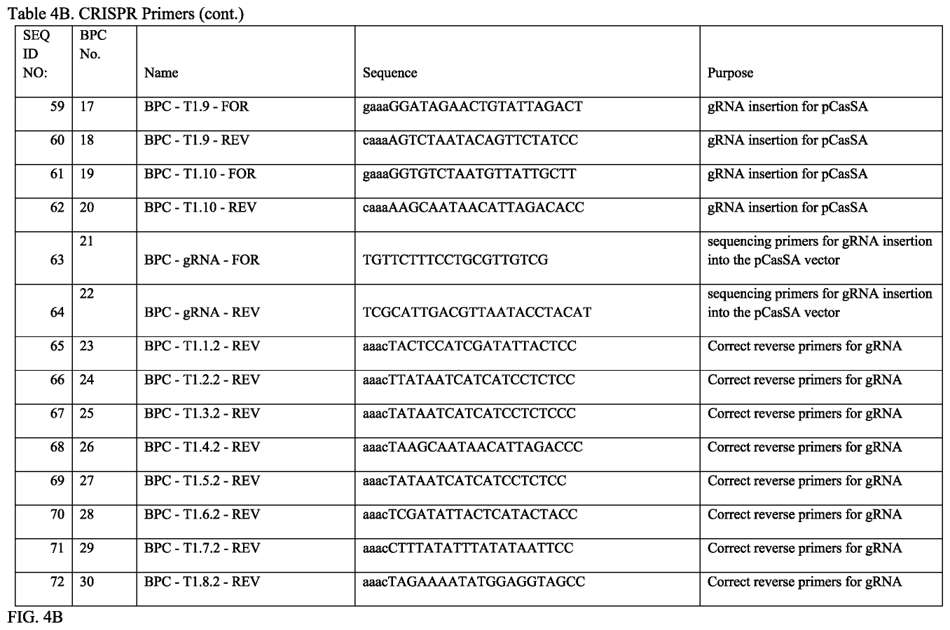

After careful consideration to the optimal integrative vector, plasmids pKOR1 or pIMAY may be employed because they provide the ability to choose the integration site, allowing us to avoid perturbing biologically critical regions of the genome that can occur with other methods. Both vectors possess a convenient means for counter-selection (secY) so that the plasmid backbone and its markers can be excised from the genome after the KS has been integrated. A genetic map of pKOR1 is shown in FIG. 5A and the features are described in Bae et al. 2006 Plasmid 55, pp. 58-63, and briefly described in Table 9. An advantage of pKOR is the ability to clone inserts without the limits of specific restriction enzymes.

TABLE 9
Purpose of elements in pKOR integrative plasmid
Integrative Plasmid pKOR

| Element | Purpose |
|---|---|
| AmpR | beta-lactamase; confers resistance to ampicillin in E. coli |
| | (but not in Staphylococcus aureus) |
| Ori (—) | E. coli origin of replication |
| Attp1 and 2 | Recombine with AttB elements of DNA inserts |
| CcdB | E. coli gyrase inhibitor protein; growth of cells containing |
| | non-recombinant plasmid are inhibited by this protein |
| Cat− and | Chloramphenicol resistance genes for use in gram neg and |
| Cat+ | gram + bacteria respectively |
| SecY570 | 570 nt encoding essential N terminus of secY; its antisense |
| | is expressed from the ATc-indicible pxyl/tetO promoter; |
| | growth in the presence of Atc means the plasmid backbone |
| | has been lost |
| RepF | Replication gene for Staphylococcus aureus |

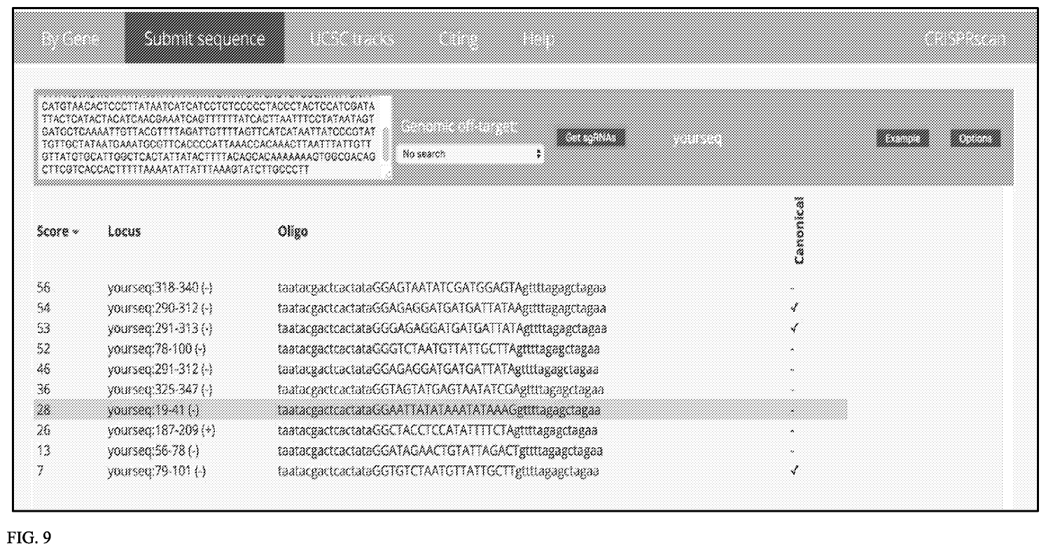

A Genetic map of pIMAY is shown in FIG. 5B from Monk, I R et al., mBio 2012; doi:10.1128/mBio.00277-11. FIG. 12A-12C shows nucleotide sequence (SEQ ID NO: 131) of pIMAY Integrative Plasmid. (accession number JQ62198). The E. coli/staphylococcal temperature-sensitive plasmid pIMAYz comprises the low-copy-number E. co/i origin of replication (p15A), an origin of transfer for conjugation (oriT), the pBluescript multiple cloning site (MCS), and the highly expressed cat gene (Phelp-cat) derived from pIMC. The temperature-sensitive replicon for Gram-positive bacteria (repBCAD) and the anhydrotetracycline-inducible antisense secY region (anti-secY) may be amplified from pVE6007 and pKOR1, respectively. The restriction sites listed are unique. Primers (IM151/152) bind external to the MCS of pIMAY and are used to screen clones in E. co/i (amplify 283 bp without a cloned insert) and to determine the presence of a replicating plasmid in staphylococci. Advantages of pIMAYz are smaller size, blue white screening, and a lower nonpermissive temperature, which has been reported to avoid mutations that can occur in the integration process. Thus, the plasmid may be made by de novo gene synthesis at a contract vendor firm.

Step 2. Review Selectable Markers in BioPlx-01.

BioPlx-01 is sensitive to ampicillin (50 μg/mL and 100 μg/mL), chloramphenicol (10 μg/mL), and erythromycin (Drury 1965). In one embodiment, the chloramphenicol (cat+) gene is used to select for transformants on chloramphenicol plates during the integration process.

Step 3. Generate the DNA Fragment to be Integrated.

Prepare a plasmid in shuttle vector pTK1 that contains the following elements in tandem: [aTTB2]-[1 Kb of sequence upstream of target region to be replaced]-[KS cassette-AmpR]-[1 Kb of sequence downstream of target region] ATTB1 according to a modification of Bae et al., 2006. Drop the fragment out of this plasmid with restriction enzymes and isolate it. The “KS cassette” may actually be one or two copies of a KS, pending the outcome of genetic stability testing.

Step 4. Insert KS Cassette(s) to pKOR Plasmid.

Perform in vitro recombination of the fragment from step 3 with the plasmid PKOR1 and then transfect the recombination mixture into DH5 alpha and obtain desired plasmid construct by standard screening methods in E. coli, using restriction mapping to verify construction.

Step 5. Obtain the KS Strain-Containing Integration Plasmid, in BioPlx-01

Electroporate the plasmid into RN4220; isolate plasmid DNA from the thus transfected RN4220, and electroporate this DNA into BioPlx-01 and select transformants on TSA plates containing chloramphenicol (10 μg/mL).

Step 6. Plasmid Integration to Chromosome.

Shift the strains to the non-permissive temperature (43° C.) to promote plasmid integration to the target site, and select a colony on a chloramphenicol plate (10 g/mL).

Step 7. Counter Selection to Evict Plasmid Backbone

Grow the colony isolate from step 6 at the permissive temperature (30° C.) to favor plasmid excision and plate on 2 μg/mL and 3 μg/mL anhydrotetracycline (aTc) agar to obtain colonies in which the target gene has integrated and the plasmid has been excised and lost (the counterselection step). Any colonies that grow on plates containing ≥2 μg/mL aTc do not contain the plasmid because the plasmid backbone contains the lethal aTc-derepressible SecY antisense gene.

Step 8. Confirm Integrated Allele Sequence

Isolate genomic DNA from the KS strain and confirm the knock-in cassette and flanking structure by PCR (and sequencing of the PCR amplicon).

Step 9. Check Serum-Induced Cell Death

Once confirmed, conduct cell death rate assays by growing the cells first in TSB, then shifting to human blood or serum and determining the rate of death by CFU plating assays in TSA (10 days).

Step 10. Verify Expression of KS mRNA

Confirm expression changes of the target gene in blood, serum, and in TSB.

Step 11. Prepare Frozen Banks

Animal studies may be performed with synthetic microorganisms BioPlx-XX created by these methods. In vivo functional studies to test kill switch strain function may be performed. Possible studies include a mouse study to show difference in pathogenicity of intravenous or intraperitoneal injection of wt BioPlx-01 vs. KS strain. An in vitro skin colonization test may also be performed. Additional tests may include, in mouse: LD₅₀ test, BioPlx-01 vs. BioPlx-XX is performed. As another example, in rat or other: colonization test, BioPlx-01 vs. BioPlx-XX is performed.

CRISPR-Cas Induced Homology Directed Repair to Direct Insertion of Optimal Kill Switch Candidates for Long Term Stable Expression

In some embodiments, a method for preparing a synthetic Staphylococcus aureus strain from BioPlx-01 is provided comprising use of CRISPR-Cas induced homology directed repair to direct insertion of optimal KS candidates for long-term stable expression. In some embodiments, a method for preparing a synthetic Staphylococcus aureus strain from BioPlx-01 is provided comprising (1) obtaining competent cells, (2) design and testing of CRISPR guide RNA (gRNA) sequences and simultaneously testing pCasSA, (3) designing and testing homology dependent repair templates using a fluorescent reporter controlled by a constitutive reporter, (4) checking KS promoters with fluorescent reporter, (5) inserting KS into BioPlx-01 and verifying incorporation, and (6) testing for efficacy and longevity. Optionally, inserting additional KS cassettes in alternative locations within BioPlx-01 genome is performed.

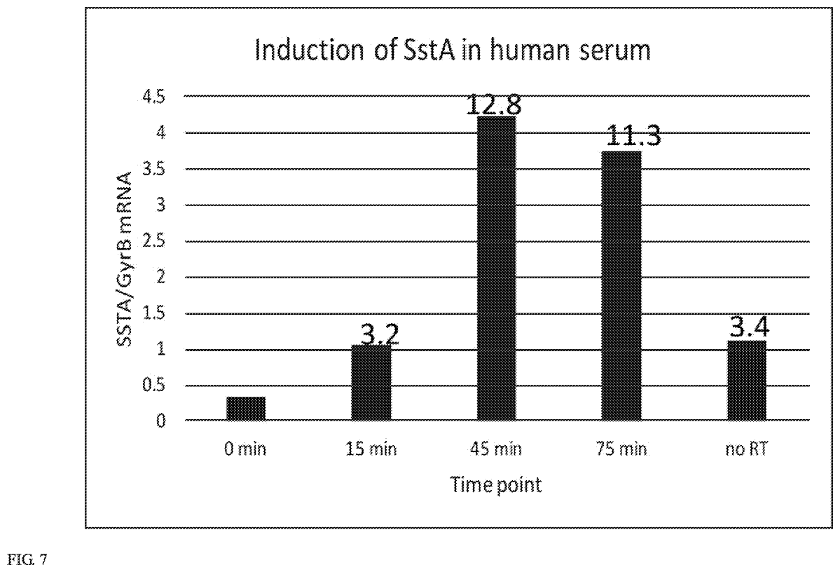

FIG. 10 shows cassette for integration via CRISPR and layout of the pCasSA vector. Pcap1A is a constitutive promoter controlling gRNA transcription. Target seq is targeting sequence, for example, with 10 possible cutting targets (1.1, 1.2 etc.). gRNA is single-strand guide RNA (provides structural component). Xbal and Xhol are two restriction sites used to add the homology arms (HAs) to the pCasSA vector. HAs are homology arms to use as templates for homology directed repair (200-1000 bp). P_{rpsL}-mCherry is a constitutive promoter controlling the “optimized” mCherry. P_{rpsL}-Cas9 is a constitutive promoter controlling Cas9 protein expression.

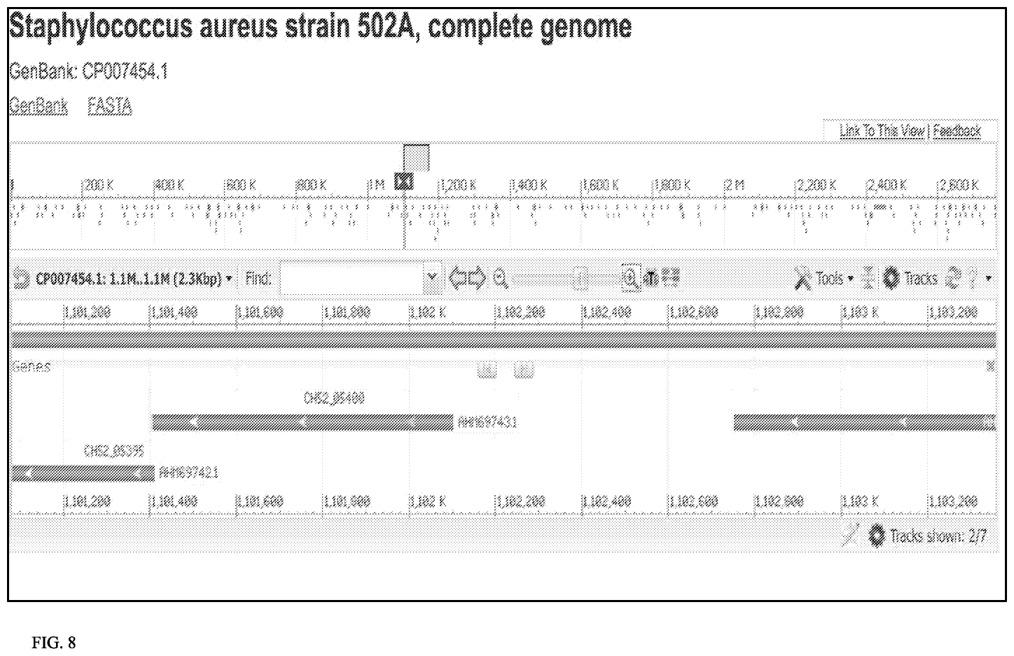

FIG. 11 shows vectors for various uses in the present disclosure. A is a vector used for promoter screen with fluorescence using pCN51. B is a vector for promoter screen with cell death gene. C is a vector for chromosomal integration using CRISPR. D is a vector for chromosomal integration using homologous recombination. L & R HA: homology arms to genomic target locus, CRISPR targeting: RNA guide to genomic locus, mCherry: fluorescent reporter protein, Cas9 protein: CRISPR endonuclease, kanR: kanamycin resistance, oriT: origin of transfer (for integration), and Sma1: representative kill gene (restriction endonuclease).

Administration and Compositions

In some embodiments, compositions are provided comprising a synthetic microorganism and an excipient, or carrier. The compositions can be administered in any method suitable to their particular immunogenic or biologically or immunologically reactive characteristics, including oral, intravenous, buccal, nasal, mucosal, dermal or other method, within an appropriate carrier matrix. In one embodiment, compositions are provided for topical administration to a dermal site, and/or a mucosal site in a subject. Another specific embodiment involves the oral administration of the composition of the disclosure.

In some embodiments, the replacing step comprises topically administering of the synthetic strain to the dermal or mucosal at least one host subject site and optionally adjacent areas in the subject no more than one, no more than two, or no more than three times. The administration may include initial topical application of a composition comprising at least 10⁶, at least 10⁷, at least 10⁸, at least 10⁹, or at least 10¹⁰ CFU of the synthetic strain and a pharmaceutically acceptable carrier to the at least one host site in the subject. The initial replacing step may be performed within 12 hours, 24 hours, 36 hours, 48 hours, 72 hours, 4 days, 5 days, 6 days, 7 days, 8 days, or 9 days of the final suppressing step.

The live biotherapeutic composition comprising a synthetic microorganism may be administered pre-partum, early, mid-, or late lactation phase or in the dry period to the cow, goat or sheep in need thereof. The composition may be administered to an intramammary, dermal, and/or mucosal at least one site in the aminal subject, and optionally adjacent sites at least once, for example, from one to 30 times, one to 20 times, one to ten times, one to six times, one to five times, one to four times, one to three times, or one to two times, or no more than once, twice, three times, 4 times, 5 times, 6 times, 8 times per month, 10 times, or no more than 12 times per month. Subsequent administration of the composition may occur after a period of, for example, one to 30 days, two to 20 days, three to 15 days, or four to 10 days after the first administration.

Colonization of the synthetic microorganism may be promoted in the subject by administering a composition comprising a promoting agent selected from a nutrient, prebiotic, stabilizing agent, humectant, and/or probiotic bacterial species. The promoting agent may be administered to a subject in a separate promoting agent composition or may be added to the microbial composition.

In some embodiments, the promoting agent may be a nutrient, for example, selected from sodium chloride, lithium chloride, sodium glycerophosphate, phenylethanol, mannitol, tryptone, and yeast extract. In some embodiments, the prebiotic is selected from the group consisting of short-chain fatty acids (acetic acid, propionic acid, butyric acid, isobutyric acid, valeric acid, isovaleric acid), glycerol, pectin-derived oligosaccharides from agricultural by-products, fructo-oligosaccarides (e.g., inulin-like prebiotics), galacto-oligosaccharides (e.g., raffinose), succinic acid, lactic acid, and mannan-oligosaccharides.

In some embodiments, the promoting agent may be a probiotic. The probiotic may be any known probiotic known in the art. Probiotics are live microorganisms that provide a health benefit to the host. In methods provided herein, probiotics may be applied topically to dermal and mucosal microbiomes, and/or probiotics may be orally administered to provide dermal and mucosal health benefits to the subject. Several strains of Lactobacillus have been shown to have systemic anti-inflammatory effects. Studies have shown that certain strains of Lactobacillus reuteri induce systemic anti-inflammatory cytokines, such as interleukin (IL)-10. Soluble factors from Lactobacillus reuteri inhibit production of pro-inflammatory cytokines. Lactobacillus paracasei strains have been shown to inhibit neurogenic inflammation in a skin model Kober at al., 2015, Int J Women's Dermatol 1(2015) 85-89. In human dermal fibroblasts and hairless mice models, Lactobacillus Plantarum has been shown to inhibit UVB-induced matrix metalloproteinase 1 (MMP-1) expression to preserve procollagen expression in human fibroblasts. Oral administration of L. plantarum in hairless mice histologic samples demonstrated that L. plantarum inhibited MMP-13, MMP-2, and MMP-9 expression in dermal tissue.

Clinically, the topical application of probiotics has also been shown to modify the barrier function of the skin with a secondary increase in antimicrobial properties of the skin. Streptococcus thermophiles when applied topically has been shown to modify the barrier function of the skin with a secondary increase in antimicrobial properties of the skin. Streptococcus thermophiles when applied topically has been shown to increase ceramide production both in vitro and in vivo. Ceramides trap moisture in the skin, and certain ceramide sphingolipids, such as phytosphingosine (PS), exhibit direct antimicrobial activity against P. acnes. Kober at al., 2015, Int J Women's Dermatol 1(2015) 85-89.

Two clinical trials of topical preparations of probiotics have assessed their effect on acne. Enterococcus faecalis lotion applied to the face for 8 weeks resulted in a 50% reduction of inflammatory lesions was noted compared to placebo. A reduction in acne count, size, and associated erythema was noted during a clinical study of Lactobacillus plantarum topical extract. Kober at al., 2015, Int J Women's Dermatol 1(2015) 85-89.

Clinical trials of topical probiotics have evaluated their effect on mucosal systems. In one study, Streptococcus salivarius was administered by nasal spray for the prevention of acute otitis media (AOM). If the nasopharynx was successfully colonized, there was significant effect on reducing AOM. Marchisio et al. (2015). Eur. J. Clin. Microbiol. Infect. Dis. 34, 2377-2383. In another trial, sprayed application of S. sanguinis and L. rhamnosus decreased middle ear fluid in children with secretory otitis media. Skovbjerg et al. (2008). Arch. Dis. Child. 94, 92-98.

The probiotic may be a topical probiotic or an oral probiotic. The probiotic may be, for example, a different genus and species than the undesirable microorganism, or of the same genus but different species, than the undesirable microorganism. The probiotic species may be a different genus and species than the target microorganism. The probiotic may or may not be modified to comprise a kill switch molecular modification. The probiotic may be selected from a Lactobacillus spp, Bifidobacterium spp. Streptococcus spp., or Enterococcus spp. The probiotic may be selected from Bifidobacterium breve, Bifidobacterium bifdum, Bifidobacterium lactis, Bifidobacterium infantis, Bifidobacterium breve, Bifidobacterium longum, Lactobacillus reuteri, Lactobacillus paracasei, Lactobacillus plantarum, Lactobacillus johnsonii, Lactobacillus rhamnosus, Lactobacillus acidophilus, Lactobacillus salivarius, Lactobacillus casei, Lactobacillus plantarum, Lactococcus lactis, Streptococcus thermophiles, Streptococcus salivarius, or Enterococcus faecalis.

The promoting agent may include a protein stabilizing agent such as those disclosed in an incorporated by reference from U.S. Pat. No. 5,525,336 is included in the composition. Non-limiting examples include glycerol, trehalose, ethylenediaminetetraacetic acid, cysteine, a cyclodextrin such as an alpha-, beta-, or gamma-cyclodextrin, or a derivative thereof, such as a 2-hydroxypropyl beta-cyclodextrin, and proteinase inhibitors such as leupeptin, pepstatin, antipain, and cystatin.

The promoting agent may include a humectant. Non-limiting examples of humectants include glycerin, sorbitol, sodium 2-pyrrolidone-5-carboxylate, soluble collagen, and dibutylphthalate.

Compositions

Biotherapeutic compositions are provided comprising a synthetic microorganism and a pharmaceutically acceptable carrier, diluent, emollient, binder, excipient, lubricant, sweetening agent, flavoring agent, buffer, thickener, wetting agent, or absorbent.

Pharmaceutically acceptable diluents or carriers for formulating the biotherapeutic composition are selected from the group consisting of water, saline, phosphate buffered saline, or a solvent. The solvent may be selected from, for example, ethyl alcohol, toluene, isopropanol, n-butyl alcohol, castor oil, ethylene glycol monoethyl ether, diethylene glycol monobutyl ether, diethylene glycol monoethyl ether, dimethyl sulphoxide, dimethyl formamide and tetrahydrofuran.

The carrier or diluent may further comprise one or more surfactants such as i) Anionic surfactants, such as metallic or alkanolamine salts of fatty acids for example sodium laurate and triethanolamine oleate; alkyl benzene sulphones, for example triethanolamine dodecyl benzene sulphonate; alkyl sulphates, for example sodium lauryl sulphate; alkyl ether sulphates, for example sodium lauryl ether sulphate (2 to 8 EO); sulphosuccinates, for example sodium dioctyl sulphonsuccinate; monoglyceride sulphates, for example sodium glyceryl monostearate monosulphate; isothionates, for example sodium isothionate; methyl taurides, for example Igepon T; acylsarcosinates, for example sodium myristyl sarcosinate; acyl peptides, for example Maypons and lamepons; acyl lactylates, polyalkoxylated ether glycollates, for example trideceth-7 carboxylic acid; phosphates, for example sodium dilauryl phosphate; Cationic surfactants, such as amine salts, for example sapamin hydrochloride; quaternary ammonium salts, for example Quaternium 5, Quaternium 31 and Quaternium 18; Amphoteric surfactants, such as imidazol compounds, for example Miranol; N-alkyl amino acids, such as sodium cocaminopropionate and asparagine derivatives; betaines, for example cocamidopropylebetaine; Nonionic surfactants, such as fatty acid alkanolamides, for example oleic ethanolamide; esters or polyalcohols, for example Span; polyglycerol esters, for example that esterified with fatty acids and one or several OH groups; Polyalkoxylated derivatives, for example polyoxy:polyoxyethylene stearate; ethers, for example polyoxyethyl lauryl ether; ester ethers, for example Tween; amine oxides, for example coconut and dodecyl dimethyl amine oxides. In some embodiments, more than one surfactant or solvent is included.

The biotherapeutic composition may include a buffer component to help stabilize the pH. In some embodiments, the pH is between 4.5-8.5. For example, the pH can be approximately 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0, including any value in between. In some embodiments, the pH is from 5.0 to 8.0, 6.0 to 7.5, 6.8 to 7.4, or about 7.0. Non-limiting examples of buffers can include ACES, acetate, ADA, ammonium hydroxide, AMP (2-amino-2-methyl-1-propanol), AMPD (2-amino-2-methyl-1,3-propanediol), AMPSO, BES, BICINE, bis-tris, BIS-TRIS propane, borate, CABS, cacodylate, CAPS, CAPSO, carbonate (pK1), carbonate (pK2), CHES, citrate (pK1), citrate (pK2), citrate (pK3), DIPSO, EPPS, HEPPS, ethanolamine, formate, glycine (pK1), glycine (pK2), glycylglycine (pK1), glycylglycine (pK2), HEPBS, HEPES, HEPPSO, histidine, hydrazine, imidazole, malate (pK1), malate (pK2), maleate (pK1), maleate (pK2), MES, methylamine, MOBS, MOPS, MOPSO, phosphate (pK1), phosphate (pK2), phosphate (pK3), piperazine (pK1), piperazine (pK2), piperidine, PIPES, POPSO, propionate, pyridine, pyrophosphate, succinate (pK1), succinate (pK2), TABS, TAPS, TAPSO, taurine (AES), TES, tricine, triethanolamine (TEA), and Trizma (tris). Excipients may include a lactose, mannitol, sorbitol, microcrystalline cellulose, sucrose, sodium citrate, dicalcium phosphate, phosphate buffer, or any other ingredient of the similar nature alone or in a suitable combination thereof.

The biotherapeutic composition may include a binder may, for example, a gum tragacanth, gum acacia, methyl cellulose, gelatin, polyvinyl pyrrolidone, starch, biofilm component, or any other ingredient of the similar nature alone or in a suitable combination thereof.

Use of biofilms as a glue or protective matrix in live biotherapeutic compositions in a method of identifying a biologically-active composition from a biofilm is described in U.S. Pat. Nos. 10,086,025; 10,004,771; 9,919,012; 9,717,765; 9,713,631; 9,504,739, each of which is incorporated by reference. Use of biofilms as materials and methods for improving immune responses and skin and/or mucosal barrier functions is described in U.S. Pat. Nos. 10,004,772; and 9,706,778, each of which is incorporated by reference. For example, the compositions may comprise a strain of Lactobacillus fermentum bacterium, or a bioactive extract thereof. In preferred embodiments, extracts of the bacteria are obtained when the bacteria are grown as biofilm. The subject invention also provides compositions comprising L. fermentum bacterium, or bioactive extracts thereof, in a lyophilized, freeze dried, and/or lysate form. In some embodiments, the bacterial strain is Lactobacillus fermentum Qi6, also referred to herein as Lf Qi6. In one embodiment, the subject invention provides an isolated or a biologically pure culture of Lf Qi6. In another embodiment, the subject invention provides a biologically pure culture of Lf Qi6, grown as a biofilm. The pharmaceutical compositions may comprise bioactive extracts of Lf Qi6 biofilm. For example, L. fermentum Qi6 may be grown in MRS media using standard culture methods. Bacteria may be subcultured into 500 ml MRS medium for an additional period, again using proprietary culture methods. Bacteria may be sonicated (Reliance Sonic 550, STERIS Corporation, Mentor, Ohio, USA), centrifuged at 10,000 g, cell pellets dispersed in sterile water, harvested cells lysed (Sonic Ruptor 400, OMNI International, Kennesaw, Ga., USA) and centrifuged again at 10,000 g, and soluble fraction centrifuged (50 kDa Amicon Ultra membrane filter, EMD Millipore Corporation, Darmstadt, Germany, Cat #UFC905008). The resulting fraction may be distributed into 0.5 ml aliquots, flash frozen in liquid nitrogen and stored at −80° C.

The pharmaceutical compositions provided herein may optionally contain a single (unit) dose of probiotic bacteria, or lysate, or extract thereof. Suitable doses of probiotic bacteria (intact, lysed or extracted) may be in the range 104 to 1012 cfu, e.g., one of 104 to 1010, 104 to 108, 106 to 1012, 106 to 1010, or 106 to 108 cfu. In some embodiments, doses may be administered once or twice daily. In some embodiments, the compositions may comprise, one of at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to 10 about 5%, by weight of the Lf Qi6 extracts.

The abbreviation cfu refers to a “colony forming unit” that is defined as the number of bacterial cells as revealed by microbiological counts on agar plates.

Excipients may be selected from the group consisting of agar-agar, calcium carbonate, sodium carbonate, silicates, alginic acid, corn starch, potato tapioca starch, primogel or any other ingredient of the similar nature alone or in a suitable combination thereof, lubricants selected from the group consisting of a magnesium stearate, calcium stearate, talc, solid polyethylene glycols, sodium lauryl sulfate or any other ingredient of the similar nature alone; glidants selected from the group consisting of colloidal silicon dioxide or any other ingredient of the similar nature alone or in a suitable combination thereof; a stabilizer selected from the group consisting of such as mannitol, sucrose, trehalose, glycine, arginine, dextran, or combinations thereof, an odorant agent or flavoring selected from the group consisting of peppermint, methyl salicylate, orange flavor, vanilla flavor, or any other pharmaceutically acceptable odorant or flavor alone or in a suitable combination thereof; wetting agents selected from the group consisting of acetyl alcohol, glyceryl monostearate or any other pharmaceutically acceptable wetting agent alone or in a suitable combination thereof; absorbents selected from the group consisting of kaolin, bentonite clay or any other pharmaceutically acceptable absorbents alone or in a suitable combination thereof; retarding agents selected from the group consisting of wax, paraffin, or any other pharmaceutically acceptable retarding agent alone or in a suitable combination thereof.

The biotherapeutic composition may comprise one or more emollients. Non-limiting examples of emollients include stearyl alcohol, glyceryl monoricinoleate, glyceryl mono stearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate.

The microbial composition may include a thickener, for example, where the thickener may be selected from hydroxyethylcelluloses (e.g. Natrosol), starch, gums such as gum arabic, kaolin or other clays, hydrated aluminum silicate, fumed silica, carboxyvinyl polymer, sodium carboxymethyl cellulose or other cellulose derivatives, ethylene glycol monostearate and sodium alginates. The microbial composition may include preservatives, antiseptics, pigments or colorants, fragrances, masking agents, and carriers, such as water and lower alkyl, alcohols, such as those disclosed in an incorporated by reference from U.S. Pat. No. 5,525,336 are included in compositions.

The live biotherapeutic composition may optionally comprise a preservative. Preservatives may be selected from any suitable preservative that does not destroy the activity of the synthetic microorganism. The preservative may be, for example, chitosan oligosaccharide, sodium benzoate, calcium propionate, tocopherols, selected probiotic strains, phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; chelating agents such as EDTA; salt-forming counter-ions such as sodium; metal complexes (e.g. Zn-protein complexes), such as m-cresol or benzyl alcohol. The preservative may be a tocopherol on the list of FDA's GRAS food preservatives. The tocopherol preservative may be, for example, tocopherol, dioleyl tocopheryl methylsilanol, potassium ascorbyl tocopheryl phosphate, tocophersolan, tocopheryl acetate, tocopheryl linoleate, tocopheryl linoleate/oleate, tocopheryl nicotinate, tocopheryl succinate. The composition may include, for example, 0-2%, 0.05-1.5%, 0.5 to 1%, or about 0.9% v/v or wt/v of a preservative. In one embodiment, the preservative is benzyl alcohol.

The compositions of the disclosure may include a stabilizer and/or antioxidant. The stabilizer may be, for example, an amino acid, for example, arginine, glycine, histidine, or a derivative thereof, imidazole, imidazole-4-acetic acid, for example, as described in U.S. Pat. No. 5,849,704. The stabilizer may be a “sugar alcohol” may be added, for example, mannitol, xylitol, erythritol, threitol, sorbitol, or glycerol. In the present context “disaccharide” is used to designate naturally occurring disaccharides such as sucrose, trehalose, maltose, lactose, sepharose, turanose, laminaribiose, isomaltose, gentiobiose, or melibiose. The antioxidant may be, for example, ascorbic acid, glutathione, methionine, and ethylenediamine tetraacetic acid (EDTA). The optional stabilizer or antioxidant may be in an amount from about 0 to about 20 mg, 0.1 to 10 mg, or 1 to 5 mg per mL of the liquid composition.

The biotherapeutic compositions for topical administration may be provided in any suitable dosage form such as a liquid, dip, sealant, solution, suspension, cream, lotion, ointment, gel, balm, or in a solid form such as a powder, tablet, or troche for suspension immediately prior to administration. The gel may be a hydrogel composition such as an alginate, such as a sodium alginate, and optionally a buffer such as HEPES (N-(2-hydroxyethyl)-piperazine-1-N′-2-ethanesulfonic acid), glycine or betaine, for example, as disclosed in US20200197301. The compositions for topical use may also be provided as hard capsules, or soft gelatin capsules, wherein the synthetic microorganism is mixed with water or an oil medium, for example, peanut oil, liquid paraffin, or olive oil. The dosage form may be coated. The coating material may be a water-miscible coating material such as a sodium alginate, alginic acid, polymethylmethacrylate, wheat protein, soybean protein, methylcellulose (MC), hydroxypropylcellulose (HPC), hydroxypropylmethylcellulose (HPMC), polyvinylacetatephthalate, gums, for example, guar gum, locust bean gum, xanthan gum, gellan gum, arabic gum, etc., for example, as described in U.S. Pat. No. 6,365,148.

Powders and granulates may be prepared using the ingredients mentioned above under tablets and capsules for dissolution in a conventional manner using, e.g., a mixer, a fluid bed apparatus, lyophilization or a spray drying equipment. A dried microbial composition may administered directly or may be for suspension in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate.

The microbial composition may exhibit a stable CFU losing less than 30%, 20%, 10% or 5% cfu over at least one, two, three months, six months, 12 months 18 months, or 24 months when stored at frozen, refrigerated or preferrably at room temperature.

Kits

Any of the above-mentioned compositions or synthetic microorganisms may be provided in the form of a kit. In some embodiments, a kit comprises a container housing live bacteria or a container housing spray dried or freeze-dried live bacteria. Kits can include a second container including media. Kits may also include one or more decolonizing agents. Kits can also include instructions for administering the composition. In certain embodiments, instructions are provided for mixing the bacterial strains with other components of the composition. In some embodiments, a kit further includes an applicator to apply the microbial composition to a subject.

Dose

In certain embodiments, a composition is provided for topical or intramammary administration that is a solution composition, for reconstitution to a solution composition, a gel composition, ointment composition, lotion composition, or as a suppository composition. In one embodiment, composition may include from about 1×10⁵ to 1×10¹² cfu/ml, 1×10⁶ to 1×10¹⁰ cfu/ml, or 1.2×10⁷ to 1.2×10⁹ CFU/mL of the synthetic microorganism in an aqueous solution, such as phosphate buffered saline (PBS). Lower doses may be employed for preliminary irritation studies in a subject.

Preferably, the subject does not exhibit recurrence of the undesirable microorganism as evidenced by swabbing the subject at the at least one site after at least 2, 3, 4, 6, 10, 15, 22, 26, 30 or 52 weeks after performing the initial administering step.

Nanofactory

In some embodiments, methods are provided to create production of a desired substance at the site of the microbiome (nanofactory). Synthetic microorganisms are provided that may comprise a nanofactory molecular modification. The term “nanofactory” refers to a molecular modification of a target microorganism that results in the production of a product—either a primary product such as a protein, enzyme, polypeptide, amino acid or nucleic acid, or a secondary product such as a small molecule to produce a beneficial effect. The product may be secreted from the synthetic microorganism or may be in the form of an inclusion body. Such nanofactory bacterial strains have the potential to provide to the host subject a wide range of durable benefits including: (i) the acquisition of cellular products and enzymes for which the host was previously deficient and; (ii) the acquisition of a delivery system of a microbially manufactured small molecule, polypeptide or protein pharmaceuticals for diverse therapeutic and prophylactic benefit. Such nanofactory bacterial strains when durably integrated into the biome as described herein would provide a useful durable alternative steady state production of product than direct product application.

Methods and synthetic microorganisms are provided herein to replace existing colonization by an undesirable microorganism with a synthetic bacterial strain comprising a nanofactory molecular modification for the production or consumption of a primary or secondary product, where the target microorganism may be a strain of Acinetobacter johnsonii, Acinetobacter baumannii, Staphylococcus aureus, Staphylococcus epidermidis, Staphylococcus lugdunensis, Staphylococcus warneri, Staphylococcus saprophyticus, Corynebacterium acnes, Corynebacterium striatum, Corynebacterium diphtheriae, Corynebacterium minutissimum, Cutibacterium acnes, Propionibacterium acnes, Propionibacterium granulosum, Streptococcus pyogenes, Streptococcus aureus, Streptococcus agalactiae, Streptococcus mitis, Streptococcus viridans, Streptococcus pneumoniae, Streptococcus anginosus, Streptococcus constellatus, Streptococcal intermedius, Streptococcus agalactiae, Pseudomonas aeruginosa, Pseudomonas oryzihabitans, Pseudomonas stutzeri, Pseudomonas putida, and Pseudomonas fluorescens, Lactobacillus crispatus, Lactobacillus gasseri, Lactobacillus jensenii and Lactobacillus iners.

The nanofactory molecular modification in a synthetic microorganism may be used to assist its host subject, i.e., a patient with a deficit of some primary (anabolic or catabolic) or secondary metabolic pathway or any other ailment stemming from the over or under abundance of some small molecule or macromolecule such as an enzyme. The nanofactory molecular modification may encode an enzyme, amino acid, metabolic intermediate, or small molecule. The nanofactory molecular modification may confer a new production (synthesis) or metabolic function into the host microbiome, such as the ability to endogenously synthesize or metabolize specific compounds, or synthesize enzymes or other active molecules to operate within the exogenous microbiome.

The microorganism will carry a nanofactory selected from a biosynthetic gene, biosynthetic gene cluster, or gene(s) coding for one or multiple enzymes under the control of a differentially regulated, inducible or constitutively regulated promoter. The synthetic microorganism comprising a nanofactory is to be administered to at the at least one site of the body be it dermal, mucosal, or other site as a singular agent or in conjunction with a second, third or fourth synthetic microorganism that help the first synthetic microorganism restore the loss of function on or in the host subject.

In one example, a synthetic microorganism comprising a nanofactory may be used for restoration of function by the production of intercellularly active factors, for example, microbial supplementation of digestive enzymes in patients with exocrine pancreatic insufficiency by secreted recombinant enzymes in the small intestine. The pancreas is a vital organ and plays a key role in digestion. Exocrine pancreatic insufficiency (EPI) is caused by prolonged damage to the pancreas, which leads to the reduction or absence of quintessential digestive enzymes in the small intestine that primarily breakdown fats and carbohydrates. The loss of these enzymes can lead to a wide breadth or symptoms and depends on the severity of the EPI. The small intestine's pH level in the proximal small intestine (duodenum) is lower than that of the distal region. This shift in environment leads to microbial niche occupation that is pH dependent. This pH dependency has naturally selected for duodenum commensal bacteria that could be molecularly modified to become synthetic microorganisms, which would intrinsically localize themselves to that region of the gastrointestinal tract. The stomach and upper two-thirds of the small intestine contain acid tolerant Lactobacilli and Streptococci (Hao, Wl, Lee Y K. Microflora of the gastrointestinal tract: a review. Methods Mol. Biol. 2004, 268, 491-502) and could be isolated from healthy donors. By knocking in recombinant lipases, amylases and/or proteases with secretory signaling sequences, the colonization of the duodenum by the synthetic microorganisms could restore digestive function in patients suffering from EPI.

In another example of a nanofactory, a synthetic microorganism comprising a nanofactory may be used for restoration of function by the production of intracellularly active factors. For example, protecting a subject suffering from phenylketonuria (PKU) by eliminating phenylalanine in the gastrointestinal tract. Phenylalanine is an essential amino acid, meaning that the human body cannot produce it and must acquire it through nourishment. Once in the body, the breakdown of phenylalanine is carried out by one protein, phenylalanine hydroxylase (PAH). The inheritable genetic disorder known as phenylketonuria (PKU) is caused by mutations in the gene coding for PAH, which results in the build up of phenylalanine in the body. One of the most common approaches to circumvent this accumulation is to avoid phenylalanine rich foods. Alternatively, a synthetic microorganism that has been molecularly modified to breakdown phenyalanine intracellularly can be introduced into the gastrointestinal tract. This synthetic microorganism constitutes a PAH nanofactory, breaking down phenyalanine before it has a chance to enter the body of the host with PKU.

In another example of a nanofactory, a synthetic bacteria may be derived from a target commensal bacteria from the skin microbiota may comprising a nanofactory molecular modification. The target commensal skin or mucosal bacterium may be, e.g., a Staphylococcus spp., Streptococcus spp., or a Cutibacterium spp. For example, Staphylococcus epidermidis may be the target microorganism because it is found in multiple dermal or mucosal environmental types. Engineering a synthetic S. epidermidis, given its ability to persist in different environments, would allow for the development and optimization of multiple kinds of delivery techniques and locations.

In one example, a synthetic S. epidermidis strain may comprise a nanofactory molecular modification to produce testosterone for men suffering from male hypogonadism. The production of testosterone could be accomplished by: (i) introduction of the entire sterol biosynthetic pathway with the additional enzymes necessary to generate testosterone, or (ii) introduction of the partial sterol biosynthetic pathway and having the necessary precursor molecules in the carrying medium, i.e., farnesol, squalene, cholesterol etc, so that testosterone could be assembled in the synthetic bacterium. In another example, a synthetic S. epidermidis strain could comprise a nanofactory molecular modification for production of nicotine; this synthetic strain could be applied as a transdermal therapy to help with smoking cessation. This synthetic strain may include a molecular modification to include one or more biosynthetic pathways found in the Solanaceae family of plants, and optionally further include a molecular modification for the enhancement of intrinsic pathways of precursor molecules, i.e., aspartic acid, ornithine etc.

In a further example of a nanofactory, a synthetic S. epidermidis strain may comprise a nanofactory molecular modification for the production of scopolamine. Scopolamine is currently delivered via an extended release transdermal patch for treatment of motion sickness and postoperative prophylaxis. This strain would need to carry the biosynthetic pathways found in the Solanaceae family of plants and possibly the enhancement of intrinsic pathways of precursor molecules.

As another example, a synthetic S. epidermidis strain may comprise a nanofactory molecular modification for the production of capsaicin to alleviate pain stemming from post-herpetic neuralgia, psoriasis or other skin related disorders.

In another example, the target microorganism is a Streptococcus mutans strain, which may have one or more of a kill switch, V-block, or nanofactory molecular modification. Dental caries and dental plaque are among the most common diseases worldwide, and are caused by a mixture of microorganisms and food debris. Specific types of acid-producing bacteria, especially Streptococcus mutans, colonize the dental surface and cause damage to the hard tooth structure in the presence of fermentable carbohydrates e.g., sucrose and fructose. Dental caries and dental plaque are among the most common diseases worldwide, and are caused by a mixture of microorganisms and food debris. Specific types of acid-producing bacteria, especially Streptococcus mutans, colonize the dental surface and cause damage to the hard tooth structure in the presence of fermentable carbohydrates e.g., sucrose and fructose. Forrsten et al, Nutrients, 2010 March; 2(3):290-298. In some embodiments, the target microorganism is S. mutans having a KS and/or a nanofactory knock out for reducing acid production in presence of sucrose, fructose, or other fermentable carbohydrates.

Further examples of nanofactory molecular modifications in a synthetic microorganism to address dermatological and cosmetic uses include: (i) hyaluronic acid production in Staphylococcus epidermidis for atopic dermatitis or dry skin, (ii) alpha-hydroxy acid production in Staphylococcus epidermidis to reduce fine lines and wrinkles as well as lessen irregular pigmentation, (iii) salicylic acid production in Cutibacterium acnes to reduce acne, (iv) arbutin production in Staphylococcus epidermidis (arbutin and its metabolite hydroquinone function as skin lightening agents by melanin suppression, (v) Kojic acid (produced by several fungi including Aspergillus oryzae) in Staphylococcus epidermidis to lighten skin pigmentation, (vi) Retinoid production by Staphylococcus epidermidis for the reduction of fine lines and wrinkles, (vii) L-ascorbic acid (Vitamin C) production in Staphylococcus epidermidis for the stimulation of collagen and antioxidant effects on the skin, (viii) copper peptide (GHK-Cu) production in Staphylococcus epidermidis for stimulation of collagen and elastin production and reduction of scar formation, (ix) alpha lipoic acid production in Staphylococcus epidermidis for beneficial antioxidant effects on the skin, and (x) dimethylaminoethanol production in Staphylococcus epidermis for reducing fine lines and wrinkles.

Cutibacterium acnes is a dominant bacteria living on the skin, and has been associated with both healthy skin and various diseases. This is another organism and niche available for enhancing and strengthening with modern molecular biology techniques. Studies have shown that the levels of C. acnes are similar between healthy skin and skin laden with acne. Dréno, B., et al. “Cutibacterium acnes (Propionibacterium acnes) and acne vulgaris: a brief look at the latest updates.” Journal of the European Academy of Dermatology and Venereology 32 (2018): 5-14. This indicates that just lowering the number of viable C. acnes on a person's skin will not help to alleviate the disease or symptoms. Instead, other strains of C. acnes or other members of the dermal and subcutaneous microbiome can be altered to mitigate the mechanisms that certain C. acnes strains use to cause disease. The isolates that showed to have the greatest association with increased acne severity also have been shown to produce higher quantities of propionic and butyric acid. Beylot, C., et al. “Propionibacterium acnes: an update on its role in the pathogenesis of acne.” Journal of the European Academy of Dermatology and Venereology 28.3 (2014): 271-278.

Another example of a nanofactory molecular modification includes another strain of C. acnes that is modified to have an increased appetite for short chain fatty acids, such as propionic and butyric acid, thereby removing the inflammatory chemical secretions from the virulent strain rendering it less toxic. The carbon rich fatty acids could be used to induce a heterologous pathway and used as precursors for vitamin synthesis or other organic compounds beneficial for the skin or microbiome that inhabits that location.

In another example, in S. epidermidis lipoteichoic acid has shown to help mitigate the inflammatory response of Propionibacterium acnes (i.e., Cutibacterium acnes) by inducing miR-143. Xia, Xiaoli, et al. “Staphylococcal LTA-induced miR-143 inhibits Propionibacterium acnes-mediated inflammatory response in skin.” Journal of Investigative Dermatology 136.3 (2016): 621-630. A synthetic microorganism comprising a nanofactory molecular modification producing lipoteichoic acid which inhibits C. acnes-induced inflammation via induction of miR-143 may be employed. The nanofactory may be used to modulate inflammatory responses by S. epidermidis at the site of acne vulgaris for management of C. acnes-induced inflammation. This pathway is just one example of a useful product that could be made from short chain fatty acids that when left alone cause inflammation and skin irritation.

In another example, inflammation and an increase in temperature are factors involved in the disease caused by C. acnes, they could be used as signals to induce previously silent heterologous pathways in an engineered strain. A temperature increase (signalling a sealed pore and progressing localized disease state) could induce in the virulent strain or another commensal microbe, the transcription and translation of a non-immune stimulating lipase (or other enzyme) that is capable of degrading the sebum to the point of reopening a clogged pore allowing the location to resume its normal growth conditions.

In a further example, a synthetic Lactobacillus spp. such as Lactobacillus crispatus, Lactobacillus gasseri, Lactobacillus jensenii or Lactobacillus iners—which are common dominant species present in the female vaginal vault may be engineered to comprises a nanocatory molecular modification that produces estradiol in the vaginal vault of post-menopausal women.

Methods and synthetic microorganisms are provided herein to replace existing colonization by an undesirable microorganism with a synthetic bacterial strain comprising a nanofactory molecular modification for the production or consumption of a primary or secondary product, for example, selected from an enzyme, nicotine, aspartic acid, ornithine, propionic acid, butyric acid, hyaluronic acid, an alpha-hydroxy acid, L-ascorbic acid, a copper peptide, alpha-lipoic acid, salicylic acid, arbutin, Kojic acid, scopolamine, capsaicin, a retinoid, dimethylaminoethanol, lipoteichoic acid, testosterone, estradiol, and progesterone.

The durable integration of a synthetic bacterial strain that is able to produce by means of a nanofactory molecular modification or synthetic addition to its genome, a substance, material, or product, or products, that are beneficial to the host at the site of the microbiome integration or at distant sites in the host following absorption may be tailored to the desired indication. Depending upon whether the synthetic nucleotide change is incorporated directly into the bacterial genome, or whether it was introduced into plasmids, the duration of the effect of the nanofactory production could range from short term (with non-replicating plasmids for the bacterial species) to medium term (with replicating plasmids without addiction dependency) to long term (with direct bacterial genomic manipulation).

Virulence Block

In some embodiments, methods are provided to replace existing colonization with a synthetic bacterial strain which cannot accept genetic transfer of undesired virulence or antibiotic resistance genes. Synthetic microorganisms are provided that may comprise a “virulence block” or “V-block”. The term “virulence block”, or “V-block” refers to a molecular modification of a microorganism that results in the organism have decreased ability to accept foreign DNA from other strains or species effectively resulting in the organism having decreased ability to acquire exogenous virulence or antibiotic resistance genes.

Methods are provided herein to replace existing colonization by an undesirable microorganism with a synthetic bacterial strain comprising a V-block molecular modification which cannot accept genetic transfer of undesired virulence or antibiotic resistance genes, where the target microorganism may be a strain of Acinetobacter johnsonii, Acinetobacter baumannii, Staphylococcus aureus, Staphylococcus epidermidis, Staphylococcus lugdunensis, Staphylococcus warneri, Staphylococcus saprophyticus, Corynebacterium acnes, Corynebacterium striatum, Corynebacterium diphtheriae, Corynebacterium minutissimum, Cutibacterium acnes, Propionibacterium acnes, Propionibacterium granulosum, Streptococcus pyogenes, Streptococcus aureus, Streptococcus agalactiae, Streptococcus mitis, Streptococcus viridans, Streptococcus pneumoniae, Streptococcus anginosus, Streptococcus constellatus, Streptococcal intermedius, Streptococcus agalactiae, Pseudomonas aeruginosa, Pseudomonas oryzihabitans, Pseudomonas stutzeri, Pseudomonas putida, and Pseudomonas fluorescens.

One of the major concerns with regard to infectious diseases is commonly called “horizontal gene transfer” with potential bacterial pathogens acquiring either exogenous virulence protein genes or antimicrobial resistance genes. The acquisition may result from transfer of these genes from other bacteria strains or species in the local microbiome environment. As it is common for invasive bacterial pathogens to initially be a part of the colonizing bacterial microbiome on skin or mucosal surfaces prior to causing disease, it would be of great practical benefit to be able to imbue these colonizing strains with the inability to accept foreign bacterial DNA into the bacterial genome. The process to accomplish this in a durably integrated synthetic bacterial strain has been termed called “virulence block.”

Such a “virulence block” manipulated strain would be able to be integrated into the microbiome after a decolonization event and then through the process of competitive exclusion, remain for a time as the dominant strain within that particular niche without reacquiring undesired virulence or antibiotic resistance characteristics. Such a concept carried out on potential pathogens within the microbiome would result in a stable microbiome which could acquire neither virulence nor antimicrobial resistance genes in the horizontal transfer manner, rendering the totality of the microbiome more robust and with lowered conversion potential.

The V-block is a molecular modification that may be employed in a synthetic microorganism in order to suppress virulence or horizontal gene transfer from an undesirable microorganism. The V-block molecular modification may be created in a target microorganism by: (i) gene knockout (excise or remove) of one or more known virulence genes, (ii) frameshift of a virulence region (adding or subtracting base pairs to ‘break’ the coding frame), (iii) exogenous silencing of virulence regions using inducible promoter or constitutive promoter (embedded in the DNA genome, but functions in RNA) —like antitoxin strategy, production of CRISPR-CAS9 or other editing proteins to digest incoming virulence genes using guide RNA which may be linked to an inducible promoter or constitutive promoter, or (iv) by a restriction modification (RM) such as a methylation system to turn the organism's ‘innate immune system’ to recognize and destroy incoming virulence genes by class of molecule. Any of these methods may be employed to in order to increase resistance to horizontal gene transfer. Gene editing methods for constructing a V-block may include NgAgo, mini-Cas9, CRISPR-Cpf1, CRISPR-C2c2, Target-AID, Lambda Red, Integrases, Recombinases, or use of Phage. The virulence block may be operably linked to a constitutive promoter in the synthetic microorganism. The virulence block molecular modification may prevent horizontal gene transfer of genetic material from a virulent microorganism.

The gene cassette conferring antibiotic resistance to strains of Staphylococcus aureus (SA) may be integrated into the recipient cell's genome at a particular site. This site could be deleted or changed in a cells genome, making the landing site no longer available for the incoming DNA sequence. This has been shown not to interfere with SA's ability to grow, and would make the acquisition of the resistance cassette by the organism much less likely to occur

The V-block molecular modifications may cause the removal or neutralization of virulence factors, resistance loci or cassettes, toxins or toxigenic functions, or other undesired attributes of the biomically integrated microorganism.

A virulence block in the form of Cas9 recognition system for sequences consistent with known virulence factors or antibiotic resistance genes in Staphylococcus aureus may be used to protect strains of Staphylococcus aureus Live Biotherapeutic Products from acquiring additional virulence factors and resistances to antibiotic classes, thus rendering them as safe as initially approved and manufactured.

CRISPR is a native adaptive immune system for prokaryotic cells that has evolved over time to help defend against phage attacks. The system uses short DNA sequences complementary to phage DNA (or any target DNA) sequences to target incoming DNA and digest the strand before it can be incorporated into the genome of the living cell. This same technology may be engineered to target DNA sequences that are non-threatening to the bacterial cell, but once acquired allow the organism to cause disease and persist in environments that were previously less habitable. Through integrating the Cas-9 enzyme into the genome, or harnessing the endogenous Cas-9 if available, it is possible to introduce into the genome constitutively expressed guide RNAs that target antibiotic resistance genes. If the targeted sequences are ever introduced to the cell through horizontal gene transfer or otherwise, the incoming DNA will be cut up and unable to integrate into the genome or produce a functional peptide. If the genes become integrated into the genome before the CRISPR-Cas system can target it, the engineered CRISPR-Cas system will find it in the genome and cut the sequences at the targeted location, thus producing a non-viable cell and stopping the spread of antibiotic resistance cassettes.

The CRISPR system can also be used to target RNA sequences with the result of silencing gene expression. Instead of recognition sequences targeting the DNA sequence of antibiotic resistance or virulence genes, the recognition sequences can be designed to target mRNA. If Cas9 and the targeting guide RNAs are constitutively expressed in a cell that receives the abxR or virulence genes, the translation will be interrupted by the engineered CRISPR system impeding protein formation and the ability of the cell to use the targeted genes.

Yet another method of gene silencing in prokaryotes that may be used to target the expression of virulence or antibiotic resistant genes is to design and constitutively express regulatory RNAs that target the mRNA transcript, usually at the RBS. These would be integrated into and constitutively expressed from the genome to create a synthetic organism. The regulatory RNA is a short sequence (>100 bp) and is complementary to the 5′ untranslated region (UTR) of the mRNA transcript of the abxR or virulence gene. The constitutive expression of the short sequences should not be metabolically taxing for the organism, and will have the result of blocking translation of the targeted mRNA into a protein. The engineered RNA will sufficiently block the cells ability to utilize the targeted antibiotic resistance gene if and when it is received through horizontal gene transfer.

DNA methylation plays many important roles in prokaryotes and eukaryotes. One feature of DNA methylation allows a cell to distinguish its own DNA from foreign DNA. This makes editing and studying many wild type strains very difficult, because the organism's methylase systems recognize transformed plasmid DNA as foreign, and chew it up before it can be transcribed or integrated. Horizontal gene transfer can occur between organisms that have very similar methylation patterns because the incoming DNA looks very similar to the recipient's own DNA and it is not digested. Since the mechanism and genes responsible for adding methyl groups to specific sequences, and those that look for and cut improperly methylated DNA are known in a variety of bacterial strains, it is possible to create a synthetic organism that is capable of having a unique methylation pattern. This would serve to make all incoming DNA appear foreign to the synthetic organism and get digested before the organism can acquire the new traits. This would serve to render the horizontal gene transfer of virulence or antibiotic resistance genes into our synthetic organism a non-issue.

A V-Block in the form of a molecular disruption of one or more bacterial genomic cassette insertion sites in the synthetic microorganism can render the synthetic microorganism unable to acquire antibiotic class resistance genes from resident bacteria species that are cohabitating the biome. Such manipulation will also prevent the acquisition of virulence genes that could increase the possibility of invasive events across the bowel wall. The gene cassette conferring antibiotic resistance to strains of Staph aureus (SA) may be integrated into the recipient cell's genome at a particular site. This site could be deleted or changed in a cells genome, making the landing site no longer available for the incoming DNA sequence. So long as the V-block is shown not to interfere with the synthetic microorganisms ability to grow, and would make the acquisition of the resistance cassette by the organism much less likely to occur.

### EXAMPLES

### Example 1. Field Studies—Exclusionary Niche Using Benign Microorganism

Clinical Studies-Suppress and Replace

A clinical study was designed to identify MRSA positive subjects, suppress the MRSA strain, replace the MRSA by administering Bioplx-01 (i.e., MSSA 502a), and periodically retesting subjects for recurrence of MRSA. The study population was largely drawn from Meerut area Medical Personnel and Medical Students. No symptomatic subjects were enrolled in the study.

This is a “proof of principle” study, being performed with largely unimproved materials and methods—any result greater that 55% non-recurrence will be considered an indication of the potential efficacy of these methods. Any result at 80% or greater non-recurrence would be considered a strong indication of the current technical strength of this approach.

Study Purpose and Primary Endpoints:

1) To determine the rate of asymptomatic Staphylococcus aureus and MRSA occurrence in the general population—Meerut, UP (North India) —and to qualify participants for further phases of this study;
2) Determine the rate of MRSA recurrence in BioPlx decolonized participants;
3) Determine the rate of MRSA recurrence in BioPlx01-WT recolonized participants;
4) Determine the durability of BioPlx01-WT in preventing MRSA recurrence (to 8 & 12 wks);
5) Acceptable study recurrence level=40%, Target recurrence level=20%.

The study results are evaluated against the published recurrence rates from peer-reviewed sources, averaging 45% recurrence, 55% non-recurrence.

Identification and solicitation of potential participants was performed with total participants enrolled and tested: n=765. Patients were drawn from the Medical Staff and Medical Students of. Meerut University Medical College—LLRM Medical College (MUMC Hospital), Harish Chandra Hospital, Murti Hospital, Silver Cross Hospital, JP Hospital, and Lokpriya Hospital, Dhanvantri Hospital, Jaswantrai Hospital. A paper disclosure, informed consent, and sign up document signed by all participants.

All 765 potential participants were swabbed (Nasal) by lab personnel. All swabs were plated onto a Staphylococcus aureus and a MRSA chromagar plate by lab personnel. All plates were incubated for 24 hours at 37° C., read and scored by the study supervisor personally. Photographs were taken of all plates at reading and labeled results.

The total Staphylococcus aureus nasal swab positive (MSSA and MRSA) participants was 162 or 21.18%, at the low end of expected rate for nasal swab only. The number of MSSA only (non-MRSA) participants was 97 or 12.68%.

The number of MRSA positive participants was 65 or 8.50% of total tested population.

The MRSA positive participants (n=65) were selected for the Efficacy Study by the study supervisor. The Staphylococcus aureus positive participants were selected for the irritation study by the study supervisor.

Efficacy Study was performed using BioPlx01-WT (10\hat{ }8) in PBS.

Confirmed MRSA positive participants (n=65) were advised as to the 12 week duration and commitment to the process. Study duration was extended to 6 months. Subjects for the Efficacy Study were divided as shown in Table 10.

TABLE 10
Efficacy Study

| | |
|---|---|
| MRSA Positives Identified | n = 65 |
| MRSA positive used in treatment groups - Decol/Recol | n = 34 |
| MRSA positive used in negative controls - Decol only | n = 15 |
| MRSA lost from study (Antibiotic use/drop-out) | n = 04 |
| MRSA positive not used | n = 12 |

Decolonization/Recolonization Process

Decolonization.

A complete decolonization is performed on participants first. Following is confirmation of MRSA eradication in key sites. The total body decolonization is done with chlorhexidine, nasal decolonization is done with mupirocin, and gargling with Listerine original antiseptic as per the “Decolonization Protocol” section. After complete course of decolonization procedure (five days), a confirmation MRSA test will be administered to verify that no MRSA is present in key areas, and an Staphylococcus aureus test will be administered to gather information about post-colonization Staphylococcus aureus levels. Participants underwent five-day decolonization process, which was administered and observed by study personnel. Dermal decolonization was performed by study personnel and included (1) full body spray application of chlorhexadine (4%), (2) nasal (mucosal) decolonization with mupirocine (2%), and (3) throat (mucosal) decolonization by application of Listerine, each once per day over 5 days. Participants undergo five-day decolonization process, administered and observed by BioPlx Pvt Ltd personnel.

Dermal—Chlorhexadine

Nasal (Mucosal) —Mupirocine

Throat (Mucosal) —Listerine

The participants undergo one full-body chlorhexidine bath that fully decolonizes the skin and hair. It is also true that chlorhexidine has a residual antibiotic activity that lasts as long as the outer layer of skin is present. A five-day waiting period ensures that the outer layer of skin has sloughed off and that when the subject is recolonized, BioPlx-01 is not being killed in the process.

Nasal Decolonization. To decolonize the nose and throat, the participants must use a five-day course of mupirocin antibiotics. This fully decolonizes the nares (nose).

Throat Decolonization. To decolonize the throat, the participants must gargle for 30 seconds every day with Original Listerine. This fully decolonizes the throat.

Successful decolonization is characterized by a negative MRSA result for nose, throat, and axilla (armpit). With successful decolonization only nasal follow-up testing is required at downstream timepoints. MRSA positive in nose or throat require second full round of decolonization procedure. Patients in this category do not proceed to next phase of study until decolonized. MRSA positive in axilla does not require second full round of decolonization and may proceed to next phase of study. Axilla site must now be included in all downstream MRSA testing.

Post-Decolonization Qualification Test N-T-H-A—Staphylococcus aureus and MRSA for each study Group (1,2,3). Swabs taken by Garg lab personnel. All swabs were plated onto a Staphylococcus aureus and a MRSA chromagar plate by Gard lab personnel. All plates were incubated in Dr. Garg's lab for 24 hours. All plates were read and scored by Dr. Garg personally. Photographs were taken of all plates at reading and labeled with Dr. Garg results. All data were recorded by BioPlx Pvt Ltd in paper and digital form. All digital data are transmitted to BioPlx, Inc. for filing and entry into the records system. This procedure was used for all steps in Efficacy Study.

Recolonization was performed with application of 1.2×10⁸ cfu/mL Bioplx-01 in phosphate buffered saline (PBS), as described below, about 15 mL once per day for two consecutive days per the following schedule:

1.2×10\hat{ }8 RECOLONIZATION AND QC TESTING was performed two days back-to-back;

POST 1.2×10\hat{ }8 RECOLONIZATION TESTING—one day;

POST 1.2×10\hat{ }8 RECOLONIZATION TESTING—one week; and

Weekly Observation—week 2 and thereafter.

Post-Decolonization Qualification Test N-T-H-A—Staphylococcus aureus and MRSA was performed for each study Group (1, 2, 3).

Weekly observations included swabs of the subjects were taken by lab personnel. Anatomical sites sampled included nares, throat, axilla, hand.

All swabs were plated onto a Staphylococcus aureus and a MRSA chromagar plate by lab personnel. All plates were incubated for 24 hours at 37° C. All plates were read and scored by the study director personally. Photographs were taken of all plates at reading and labeled with results.

Negative controls. Post decolonization negative controls n=15; ID #s: 0021, 0022, 0060, 0512, 0704, 0724, 0731, 0218, 0234, 0239, 0249, 0302, 0327, 0037, 0221. Post decolonization MRSA recurrence n=15: Initial negative control run (sheet week 4—Post-Decolonization average week 6) included MRSA positive n=08; MRSA negative n=07, resulting in Recurrence=53%. A Final Negative Control run (sheet week 12—Post-Decolonization average week 16) resulted in MRSA positive n=09; and MRSA negative n=06, with a recurrence=60%.

Treatment Groups 1, 2, 3. Decolonized/Recolonized (8\hat{ }10 cell concentration): 34. The Decolonized/Recolonized was divided into three groups for the study: GROUP 1 BioPlx01-WT (10\hat{ }8) in PBS n=10; ID #s: 0015, 0086, 0146, 0147, 0149, 0155, 0178, 0625, 0657, 0667. GROUP 2 BioPlx01-WT (10\hat{ }8) in PBS n=10; ID #s: 0063, 0075, 0124, 0138, 0172, 0325, 0444, 0478, 0483, 0538; and GROUP 3 BioPlx01-WT (10\hat{ }8) in PBS n=14 ID #s: 0064, 0112, 0158, 0232, 0336, 0488, 0497, 0498, 0499, 0552, 0574, 0692, 0725, 0735.

Post Decolonization/Recolonization MRSA Recurrence: 0; GROUP 1=0; GROUP 2=0; GROUP 3=0. Duration of post decolonization MRSA negative: 18 weeks=16 cases: 0 recurrence; and 17 weeks=18 cases: 0 recurrence.

Detectable Recolonization Performance

Subjects in the efficacy study were tested for Staphylococcus aureus positive results to detect presence of replacement BioPlx 01 WT using penicillinase disks. Results are shown in Table 11.

TABLE 11
Staphylococcus aureus Positives (NvTvHvA)

| | Day/Week Post |
|---|---|
| SA positives | Colonization; +/total |
| 97.1% (Group 1 & 2 & 3) | 01 day; 33/34 |
| 91.2% (Group 1 & 2 & 3) | 01 week; 31/34 |
| 100% (Group 1 & 2 & 3) | 02 week; 34/34 |
| 97.1% (Group 1 & 2 & 3) | 03 week; 33/34 |
| 91.2% (Group 1 & 2 & 3) | 04 week; 31/34 |
| 100% (Group 1 & 2 & 3) | 05 week; 34/34 |
| 88.2% (Group 1 & 2 & 3) | 06 week; 30/34 |
| 79.5% (Group 1 & 2 & 3) | 08 week; 27/34 |
| 67.7% (Group 1 & 2 & 3) | 10 week; 23/34 |
| 85.3% (Group 1 & 2 & 3) | 12 week; 29/34 |
| 100% (Group 1 & 2& 3) | 14 week; 20/20 |

The study duration was extended to six months. At the conclusion of the study, Staphylococcus aureus positives were 100% showing a greater than 26 week total exclusionary effect of the BioPlx-01 MRSA decolonization/recolonization process with the BioPlx product as opposed to prior literature demonstrating 45% recurrence of Staphylococcus aureus nasal colonization at 4 weeks and 60% at 12 weeks with the standard decolonization method alone.

Irritation Studies

As described above, MRSA positive participants were selected for the Efficacy Study by the study supervisor (Dr. Garg). Staphylococcus aureus positive participants were selected for the Irritation Study by the study supervisor. MRSA patients require a lot of effort to screen for, so an attempt was made to preserve them for the main efficacy evaluation of the study. Non-MRSA positive colonization rates are about 33%-66% of all screened participants, so there was a more plentiful supply of them. Because MRSA is an antibiotic resistant strain of Staphylococcus aureus, testing for irritation in Staphylococcus aureus positive participants is equivalent to testing for irritation in MRSA positive participants.

Irritation studies were performed on 55 Staphylococcus aureus positive subjects by topically administering about 5 mL of BioPlx-01 (502a), at 1.2×10⁷ CFU/mL in PBS, to the right forearm. The left arm served as a negative control. Forearms were observed and photographed by study personnel at day 1, day 4 and day 7 post-application for redness or pustule development. No suppression step was performed during the irritation study. No irritation or adverse events were observed.

Culture Conditions

The efficacy studies used BioPlx-01 (1.2×10⁸ CFU/mL) in PBS (Fisher) BP2944100 phosphate buffered saline tablets dissolved in water to provide 100 mM phosphate buffer, 2.7 mM KCl and 137 mM NaCl, pH 7.4 at 25° C.

Master stocks were prepared as follows. BioPlx-01 strain was streaked onto tryptic soy agar (TSA) plates in quad streak fashion. After 20 h at 37° C., a fresh bolus of cells was used to aseptically inoculate a flask of sterile tryptic soy broth (TSB). This culture was incubated at 37° C. with agitation at 250 rpm for 18 h. Sterile 50% glycerol was added to the culture to 5% (v/v) final and the batch was aliquoted into sterile 50 mL polypropylene screwcap tubes. The aliquots were frozen at −20° C. For quality control, one aliquot was thawed, fully resuspended by vigorous shake-mixing, and diluted for the determination of colony forming units (CFU) per mL by incubation on Brain Heart Infusion (BHI) agar plates for 18 h at 37° C. CFU values were calculated from dilution-corrected colony counts. A batch of the concentrated BioPlx-01 master stock produced in this way contained 8×10⁹ CFU/mL of BioPlx-01. The phenotypic identity of the strain was confirmed by incubation on HiChrom staphylococcal chromogenic indicator medium for 18 h at 37° C., which produced only the expected green colonies. The material did not produce colonies when incubated on MRSA chromogenic indicator plates.

Preparation of Working Stock for the Efficacy Study 1.2×10⁸ CFU/mL

One 10 mL aliquot of concentrated BioPlx-01 stock that is at 8×10⁹ CFU/mL was completely thawed and then shaken for a full 1 minute to mix. 8.5 mL of this solution were added to 275 mL of sterile (room temperature) PBS, generating a 2.4×10⁸ CFU/mL stock. This was mixed well by inversion and stored at 4° C. until use. As used in the efficacy studies, to provide PBS matrix 1.2×10⁸ working solution-BioPlx-01, a vial of the “2.4×10⁸ CFU/mL” solution was mixed by vigorous inversion and 200 mL of it was added to 200 mL PBS to create a “1.2×10⁸ CFU/mL working solution-BioPlx-01”. This latter solution was the material applied to subjects in efficacy studies. The bottle was tightly capped, mixed by shaking, and stored at 4 C until use.

### Example 2. Selection of One or More Inducible Promoters

In this example, promoter candidates were evaluated. The fold-induction and basal expression of 6 promoter candidates in a MSSA strain BioPlx-01 were evaluated by incubation with human whole blood and serum. Expression was normalized to a housekeeping gene (gyrB) and was compared with that in cells growing logarithmically in liquid tryptic soy broth (TSB) media.

The BioPlx-01 was grown to mid log phase (2 OD/mL) and then washed in large volume and shifted to freshly collected serum and heparinized blood from donor TK.

The samples were incubated in slowly agitating vented flask at 125 rpm; and samples were removed for RNA isolation at 15, 45, or 75 min at 37° C. The collected bacteria were washed, and RNA was extracted using Qiagen Allprep kit, eluted and the RNA frozen. Coding DNA (cDNA) was prepared from RNA and target gene expression evaluated by real time PCR (Tagman) in an ABI 7500 Fast instrument.

Relative RNA levels were determined by interpolation against a standard curve run on a common cDNA sample that was serially diluted and tested with primer/probes specific for ORFs driven by each of 5 putative serum-responsive promoters (P_{hlgA}, P_{leuA}, P_{sstA}, P_{sirA}, P_{isdA}) and one probe for a candidate gene that is upregulated in Staphylococcus aureus on the skin during colonization, but not reported to be upregulated in blood, for use in an expression clamp strategy (P_{clfB}).

Expression of all genes was normalized to the housekeeping gene gyrB (a gyrase subunit) widely used for this purpose in Staphylococcus aureus. Ct was determined by rt PCR. Ct, PCR threshold cycle, is the cycle number at a given fluorescence; the higher the gene (mRNA) quantity, the lower the Ct.

Preliminary results using serum of a single donor are shown in Table 12.

TABLE 12
Effect of Serum exposure on activation of KS promoter candidates
in BioPlx-01 and basal expression levels in TSB

| | Fold-induction increase in | Basal Expression |
|---|---|---|
| | expression in serum treated | LeuA/GyrB ratio |
| Gene | samples by real time PCR | in TSB |
| hlgA (gamma hemolysin) | 30 | 0.19 |
| leuA (AA biosynthetic | 7.7 | 0.75 |
| enzyme) | | |
| sstA (iron transport) | 12.8 | 0.33 |
| sirA (iron transport) | 1.2 | 0.95 |
| isdA (heme transporter) | 1.7 | 0.59 |
| clfB (clumping factor B) | 1.3 | 0.78 |

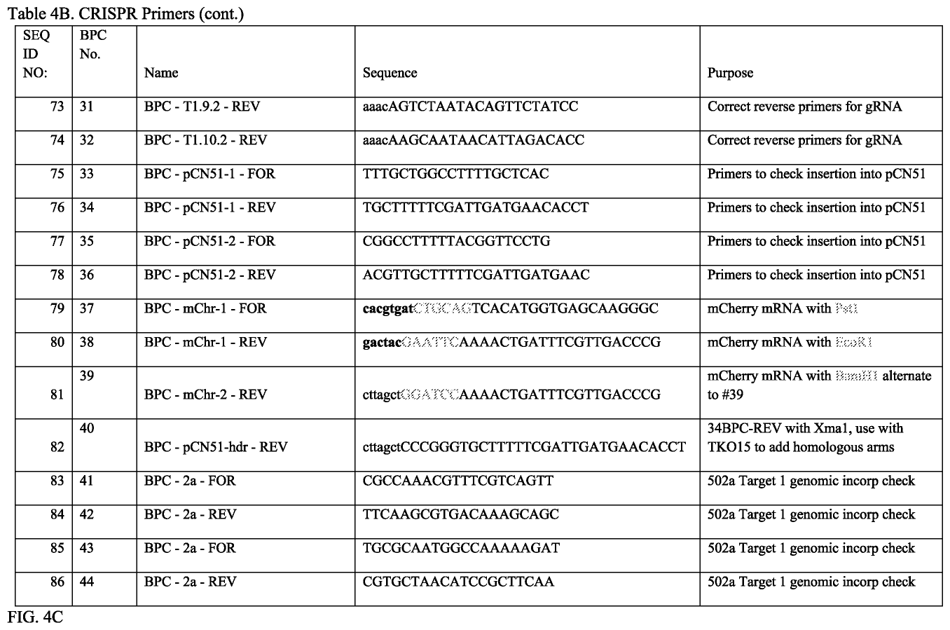

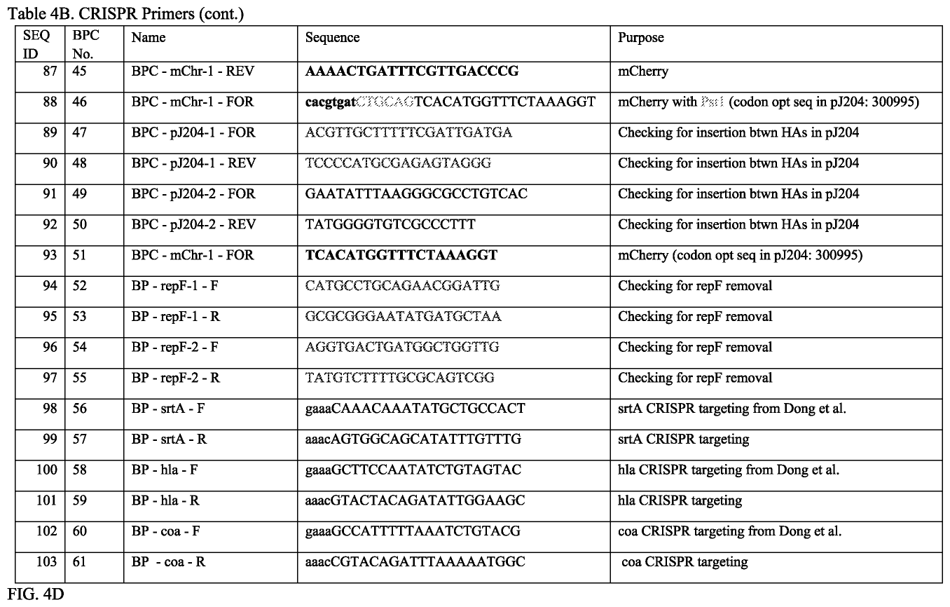

The time course of induction of promoter candidate P_{hlgA} in human serum is shown in FIG. 6 showing a hlgA/gyrB ratio in TSB of 0.19, favorable for use in kill switch construct. “no RT”: cDNA made from 75 min timepoint RNA was diluted into a reaction at same dilution as all other samples; if RNA preparation is devoid of gDNA, no signal should be visible. The time course of sstA in human serum is shown in FIG. 7 showing sst/gyrB ratio in TSB was 0.33. P_{hlgA} and P_{sstA} were selected as preliminary preferred candidates for further evaluation. hlgA levels in TSB were only ⅕^{th} of housekeeping gene gyrB, or lower, in TSB so this promoter became a lead candidate.

The experiment was repeated using serum and whole blood from two donors with analysis of total RNA, except that cDNAs were treated with DNaseI to remove contaminating genomic DNA. Specifically, RNA Samples were treated with the turbo DNAse kit following the kit protocol for treatment with and inactivation of Dnase. The “No Reverse Transcription” control (No RT control) —with DNAse was at bkg/baseline level, thus acceptable.

The treated RNA was then used to produce cDNA (and a no RT control was again run). The cDNA was analyzed (starting with hlgA and sstA) by Tagman in with technical triplicates. Results are shown in Table 13.

TABLE 13
Promoter Selection- Effect of Serum and Blood exposure on activation of
KS promoter Candidates in BioPlx-01 and Basal expression levels in TSB.

| | | | | | Serum |
|---|---|---|---|---|---|
| | Fold Induction at 15 min | | | “Leaky”expression | induction >3 |
| | Serum | Serum | Blood | Target/GyrB | fold through |
| Promoter | (donor 1) | (donor 2) | (donor 2) | (TSB) | 75 min? |
| ISDA | 83 | 15 | 4.1 | 0.002, 0.022 | yes |
| SSTA | 9 | 6.7 | 0.3 | 0.16, 0.333 | yes |
| LEUA | 1393 | 1601 | 990 | 0.0013, 0.000017 | yes |
| HLGA | 27 | 6 | 35 | 0.23, 0.26 | yes |
| SIRA | 5.5 | 2.6 | 0.08 | 0.25, 1.1 | no |

P_{isdA}, P_{sstA}, P_{sirA} were eliminated based on data shown in Table 13. P_{sstA} was eliminated because of significant basal expression, and it was not induced in whole blood. P_{sirA} was also eliminated because of significant basal expression, and low magnitude induction in serum, and was not induced in whole blood, as well as exhibiting induction that was not sustained.

Based on this experiment, P_{leuA} was selected as one preferred promoter because it exhibited very high upregulation in serum, very low basal expression in TSB, and was not upregulated during colonization. An expression clamp may be employed, but may be optional when using P_{leuA} as a promoter. P_{leuA} also exhibited strong activation by blood or serum exposure in Malachawa 2011 (microarrays) and in the present example. leuA is part of a nine-gene Operon: ilvDBHC, leuABCD, ilvA. A factor called Cody binds the RR to repress transcription when it is bound to branched chain amino acids (leucine, isoleucine and valine), so when free amino acid levels are above a threshold, the promoter is silent. In porcine ex vivo nasal colonization assays with MRSA, amino acid biosynthetic operons including leu were not upregulated, and the authors propose that amino acids are present in sufficient quantity during colonization to prevent upregulation of these pathways (Tulinski et al., 2014).

The gene leuA is activated very strongly in blood and serum and has low basal expression, so further understanding is important. leuA is within the second of two cassettes in a nine-gene operon; the regulatory region driving it may be immediately upstream of ilvD or upstream of leuA. One way to understand is to test and compare both variants.

ilvDBHC-leuABCD-ilvA

P_{hlgA} was selected as another preferred promoter because it exhibited high upregulation in serum and blood, and downregulation during nasal colonization. One drawback of P_{hlgA} is basal expression in TSB; which may be addressed by including an expression clamp for hlgA. The peptide HlgA is a subunit of a secreted, pore-forming toxin that lyses host red blood cells and leukocytes. HlgA (class S) associates with HlgB (class F) thus forming an AB toxin in strains producing both gamma-hemolysins and leukocidins (HlgA and LukF-PV can also form a complex).

Transcription of the HlgA operon is upregulated in TSB by quorum sensing agr activation, but agr is downregulated in serum while hlgA is upregulated, so hlgA upregulation is independent of the agr pathway in serum. In one paper, the hemolysins were downregulated 5.7 fold compared with TSB during colonization, specifically, porcine nasal explants colonized with MRSA ST398; see Tulinski et al 2014. However, in these experiments, no evidence of expression of hlgA was seen during colonization. The regulator sarT represses transcription of the hemolysin operon and may be a useful “expression clamp” if P_{hlgA} is used to drive the KS, for example by overexpression of sarT from a colonization promoter.

In another embodiment, a synthetic microorganism comprises at least one molecular modification comprising a first cell death gene operably linked to a first regulatory region comprising a multiplicity of promoters that are activated by serum or blood, but exhibits little to no expression in human skin, mucosa, or in TSB. There is more certainty of lower expression on skin for hlgA, because it is downregulated in colonization. There is more certainly of lower expression in TSB for leuA.

### Example 3. Selection of One or More Death Genes

In this example, cell death gene candidates are evaluated for preparing a synthetic microorganism having at least one molecular modification comprising a first cell death gene operably linked to a first regulatory region comprising a first inducible promoter. Relative potencies of death genes are unknown. What appears to be the best death gene is not necessarily the most potent one because of leaky expression. Diversity of mechanism of action could result in killing synergy for two or more death gene combinations. Death gene candidates include: SprA1: membrane disruption; sma1: genome destruction; and rsaE: blocks central metabolism. Various combinations of death genes are shown in Table 14. These plasmids are created and sequenced plasmids for testing of P_{leuA} and P_{hlgA}-driven KS variants.

TABLE 14
Death Gene KS Constructs

| Strain # | Plasmid name | Promoter | Kill gene (PCD) | Purpose | Comments |
|---|---|---|---|---|---|
| 1 | pTK1 | Cadmium | SprA1 | +control | Cells in TSB |
| | | inducible | | shows | treated with |
| | | | | sprA1SprA1 | Cd should |
| | | | | is a functional | rapidly die |
| | | | | kill gene | |
| 1A | pTK2 | Cadmium | sprA1SprA1 | Neg control | Cells in TSB |
| | | inducible | reversed | | treated with |
| | | | | | Cd should NOT |
| | | | | | rapidly die |
| 2 | pTK3 | LeuA | sprA1SprA1 | KS | Cells shifted |
| | | | | | to serum or |
| | | | | | blood should |
| | | | | | rapidly die |
| 3 | pTK4 | LeuA | sprA1SprA1 | plasmid more | Compare |
| | | | reverse | readily | Insertion |
| | | | | obtainable | frequency to |
| | | | | than # 2 | # 2 |
| 4 | pTK5 | LeuA | sprA1SprA1 + | KS | Expression |
| | | | CLFB:: | | clamp variant |
| | | | sprA1SprA1 as | | of # 2 |
| 5 | pTK6 | HlgA | sprA1SprA1 | KS | Might not be |
| | | | | | healthy or |
| | | | | | even |
| | | | | | obtainable- |
| | | | | | basal exp |
| 6 | pTK7 | HlgA | SprA1 + | KS | Likely |
| | | | CLFB::SprA 1 as | | healthier than |
| | | | | | # 5 |
| 7 | pTK8 | Cadmium | sma1 restriction | +control | Cells in TSB |
| | | inducible | enzyme | | treated with |
| | | | | | Cd should |
| | | | | | rapidly die |
| 8 | pTK9 | HlgA | sma1 restriction | KS | expression |
| | | | enzyme | | clamp made |
| | | | | | using antisense |
| 9 | pTK10 | LeuA | sma1 restriction | KS | expression |
| | | | enzyme | | clamp made |
| | | | | | using antisense |
| 10 | pTK11 | Cadmium | rsaE sRNA | +control | Cells in TSB |
| | | inducible | | | treated with |
| | | | | | Cd should |
| | | | | | rapidly die |
| 11 | pTK12 | HlgA | rsaE sRNA | KS | expression |
| | | | | | clamp made |
| | | | | | using antisense |
| 12 | pTK13 | LeuA | rsaE sRNA | KS | expression |
| | | | | | clamp does |
| | | | | | not exist but |
| | | | | | could be made |
| | | | | | using antisense |

Death genes may be obtained commercially (Atum) and vector may also be obtained commercially (BEI). Combinations comprising two death genes are constructed after results of single death genes are obtained. Synthetic plasmids, vectors and synthetic microorganisms are prepared based on Table 14.

Steps in creating a synthetic strain comprising a cell death gene are as follows.

1. Produce shuttle vector pCN51 in mid-scale in E. coli.

2. Clone death genes into pCN51 in E coli (under Cd-inducible P_{cad}).

3. Replace P_{cad} with serum-responsive promoters; and insert expression clamp where applicable.

4. Verify constructions by sequencing the KS cassettes.

5. Electroporate into Staphylococcus aureus RN4220 and select transformants on erythromycin plates (this strain is restriction minus and generates the right methylation pattern to survive in BioPlx-01). RN4220 is and Staphylococcus aureus strain used as an intermediate; restriction minus, methylation+; BEI product number NR-45946.

6. Prepare plasmid from RN4220 and restriction digest to confirm ID.

7. Electroporate plasmids into BioPlx-01 and select on erythromycin plates.

8. Synthetic microorganism strains ready for serum experiment.

Steps in testing a synthetic microorganism strains having at least one molecular modification comprising a first cell death gene operably linked to a first regulatory region comprising a first promoter are as follows.

1. Growth in TSB plus antibiotic as selective pressure for plasmid.

2. How does growth compare with WT Bioplx-01? Prepare growth curve.

3. Cd-promoter variants: Wash and shift cells to Cd medium (control is WT Bioplx-01 containing empty vector with no death gene).

4. KS variants: Wash and shift cells to serum (control is WT Bioplx-01 containing empty vector with no death gene).

5. Monitor growth using OD_{630 nm} with plate reader (extended period, monitor for appearance of escape mutants).

6. For whole blood test, only perform on winning candidates and use CFU on TSB agar as death readout.

7. If there are apparent escape mutants, shuttle plasmid out to E. coli and sequence the whole plasmid.

Plasmids may be prepared from commercially available products. In one embodiment, pCN51 (6430 bp) is the commercial plasmid for modification. pCN51 is an E. coli-SA shuttle vector, with ampR for E. coli selection and ermC for Staphylococcus aureus selection. This is a pT181 based low copy rolling circle plasmid, containing a Cadmium inducible promoter and BLA terminator. BEI product number NR-46149. Combinations of KS variants are possible in one plasmid. It is possible to insert more than one KS into the MCS of a shuttle vector plasmid.

1. The 3 constructs encoding the 3 kill genes are ordered from Atum/DNA2.0, with restriction suites placed strategically at ends of each gene for directional cloning.

2. pCN51 shuttle vector (BEI NR-46149), RN4220 Staphylococcus aureus (BEI NR-45946), and DC10B E. coli (BEI NR-49804) are ordered from BEI Resources.

3. The DNA oligonucleotides shown in Table 15 are ordered from for: i) PCR amplification of RRs from BioPlx-01 gDNA, with restriction enzymes at ends for directional cloning, and; ii) DNA sequencing of KS constructs.

TABLE 15
Oligonucleotides used for sequencing KS constructs

| Oligo | | |
|---|---|---|
| Name | Sequence (5′ to 3′) | Purpose |
| TKO1 | gatgcGCATGCGAAACAGATTATCTATTC (SEQ ID | P_{leuA} PCR Amplification with |
| | NO: 9) | SphI (upstream pr) |
| TKO2 | gatgcGCATGCCAGATTATCTATTCAAAG | P_{leuA} PCR Amplification with |
| | (SEQ ID NO: 10) | SphI (upstream pr-alternate) |
| TKO3 | catgatCTGCAGAGTAAATTCCCCCGTAAATT | P_{leuA} PCR Amplification with |
| | (SEQ ID NO: 11) | PstI (downstream pr) |
| TKO4 | cacgtgatCTGCAGAGTAAATTCCCCCGTAAA | P_{leuA} PCR Amplification with |
| | (SEQ ID NO: 12) | PstI (downstream pr-alternate) |
| TKO5 | gactacGAATTCAGGTGATGAAAAATTTAGAA | upstream primer to amplify |
| | (SEQ ID NO: 13) | P_{clfB} with EcoRI |
| TKO6 | gactacGAATTCTGATGAAAAATTTAGAACTT | backup to TKO5 |
| | (SEQ ID NO: 14) | |
| TKO7 | cttagctGGATCCAAATATTACTCCATTTCAA | downstream primer to amplify |
| | (SEQ ID NO: 15) | P_{clfB} with BamHI |
| TKO8 | cttagctGGATCCAAATATTACTCCATTTCAATTTC | backup to TKO7 |
| | (SEQ ID NO: 16) | |
| TKO9 | gatgcGCATGCTCACAAACTATTGCGAAATC | upstream primer to amplify the |
| | (SEQ ID NO: 17) | P_{hlgA}; contains SphI |
| TKO10 | gatgcGCATGCAAACTATTGCGAAATCCATTC | backup to TKO9 |
| | (SEQ ID NO: 18) | |
| TKO11 | catgatCTGCAGATATATAATAATCCATTTGT | downstream primer to amplify |
| | (SEQ ID NO: 19) | P_{hlgA}; contains PstI |
| TKO12 | catgatCTGCAGATATATAATAATCCATTTGTAAGCG | backup to TKO11 |
| | (SEQ ID NO: 20) | |
| TKO13 | GTGTTACGATAGCAAATGCA | First sense primer for |
| | (SEQ ID NO: 21) | sequencing constructs |
| | | containing P_{cad} |
| TKO14 | TTATTGGCTAAGTAGACGCA | second sense sequencing |
| | (SEQ ID NO: 22) | primer anneals roughly in the |
| | | middle of the sprA1 gene |
| TKO15 | CACATGTTCTTTCCTGCGTT | primer to anneal just upstream |
| | (SEQ ID NO: 23) | of the serum responsive P_{leuA} |
| | | and P_{hlgA}. Anneals in the |
| | | pCN51 vector about 75 nt |
| | | upstream of the SphI site |
| TKO16 | ACGCGGCCTTTTTACGGTTC | backup for TKO15 |
| | (SEQ ID NO: 24) | |
| TKO17 | GAATGGGACTTGTAAACGTC | primer to anneal near the |
| | (SEQ ID NO: 25) | downstream one third of the |
| | | P_{leuA} because its a fairly large |
| | | segment and TKO15 may not |
| | | read all the way through |
| TKO18 | GAATGGGACTTGTAAACG | backup for TKO17 |
| | (SEQ ID NO: 26) | |
| TKO19 | ATAAACGCCTGCGACCAATA | primer to anneal near the |
| | SEQ ID NO: 27) | downstream one third of the |
| | | P_{hlgA} because its a fairly large |
| | | segment and TKO15 may not |
| | | read all the way through |
| TKO20 | GCGACCAATAAATCTTTTAA | Backup for TKO19 |
| | (SEQ ID NO: 28) | |

Cloning

All gel-electrophoresis agarose gels are 1.0-2.0% agarose in 1×TAEL buffer and midori green (Nippon Genetics Europe GmbH) added per the manufacturer's instructions.

### Example 3A. Constructing pTK1 and pTK2

1. Prepare Miniprep Quantities of pCN51 and of sprA1, Sma1, and rsaE Plasmids as Follows

A. Streak the strains on LB+ carbenicillin (100 μg/mL) plates and incubate 15-18 h at 37° C.
B. Inoculate LB+ carbenicillin (100 μg/mL) liquid with single colony of each and incubate with agitation (240 rpm) for 15-18 h at 37° C.
C. Prepare 5× replicate minipreps of each strain with Qiagen spin miniprep kit per manufacturer's instructions, elute DNA from each column with 30 μL, and pool the replicate plasmid preps together (freeze DNA at −20° C.).

2. Digests, Ligation, Plating

2.1. Cut pCN51 with PstI and EcoRI to linearize (37° C., 30 mins). Expected size is ˜6400 bp (a 35 bp fragment from the multiple cloning site (MCS) is dropped out/not visible on gel).
2.2. Cut pCN51 plasmid with Kpn1 and BamHI to linearize (37° C., 30 mins). Expected size is 6400 bp (a 35 bp fragment from the MCS is dropped out).
2.3 Cut sprA1 plasmid from DNA2.0 with Pst1 and EcoRI to liberate the desired 233 bp sprA1 insert.
2.4 Cut sprA1 plasmid from DNA2.0 with Kpn1 and BamHI to liberate the desired 233 bp sprA1 insert.
2.5. During DNA digestion pour a gel that is 1.5% agarose gel for electrophoresis as described.
2.6. Add 8 μL of 6× loading dye to all 4 reactions and to the 1 kb plus DNA size ladder (3 μL in 30 μL).

### 2.7. Run gel at 100 V for 1.5 h.

2.8. Excise the bands of interest mentioned above with a clean razor blade.
2.9. Melt the slices in 3 volumes of buffer QG from Qiagen gel extraction kit (56° C.), vortexing occasionally.
2.10. Isolate the paired vector and insert together on one column and elute the material into 30 μl of Qiagen's elution buffer.
A. Pst1+EcoRI insert plus pCN51 Pst1/EcoRI vector.
B. Kpn1+BamHI insert plus pCN51 Kpn1/BamHI.
2.11 Set up a waterbath by adding some ice to 500 mL RT water in a styrofoam box; add just enough ice to reach 16° C.
2.12. Add 3.4 μL of 10× T4 DNA ligase buffer and mix. Add 1 μL of T4 DNA ligase (4×10⁵ U/mL stock from NEB) and incubate for 2 h at 16° C.
2.13 Set electroporation unit to 1500 V/200 ohms/25 μF.
2.14. Thaw 2 vials of DH5a E. coli and add 40 μL into each into 2 Eppendorf tubes. Chill 2 electroporation cuvettes on ice.
2.15. Add 1 μL of undiluted ligation to 40 μL of the thawed DH5α E. coli and transfer to an ice-cold 1 mm gap electroporation cuvette.
2.16. Have ready: 1 mL of SOC medium in a 1 mL pipet, sterile 1 mL tips, and 2 sterile 14 mL culture tubes
2.17. Electroporate the cells (ligation A first) and then ASAP add 1 mL SOC to the cuvette, pipet up and down 6×, and transfer the whole volume to a fresh 14 mL culture tube for recovery. Repeat this process for electroporation of ligation B. Place the two recovering samples in the shaking water bath at 37° C. for 1 h.
2.18. Place 2 LB+carbenicillin (100 μg/mL) agar plates inverted with their lids slightly off in the 37° C. incubator (not humidified) while the cells recover
2.19. After the 1 h recovery period, remove and label the LB+carbenicillin (1050 μg/mL) agar plates accordingly and remove the 14 mL tubes from the waterbath.
2.20. Using a sterile glass beads, spread 150 μL of each 1 mL recovery mix onto a plate.
2.21 Place the plates in the 37° C. incubator for 16-18 h.
2.22. Record colony counts for
Ligation A (P_{cad}::sprA1 forward) and
Ligation B (P_{cad}::sprA1 reverse).

3. Screening for Positives:

3.1 Pick 6 colonies for screening
3.2 Inoculate 6 colonies of ligation A and 6 of ligation B, each into 3 mL of liquid LB+carbenicillin (1050 μg/mL) in a 14 mL culture tube.

### 3.3 Shake for 16 h at 37° C.

3.4 Isolate plasmid DNAs using Qiagen spin mini kit per manufacturer's instructions, and elute DNA into 40 μL elution buffer.
3.5 Digest 5 μL of each of the 12 plasmid DNAs with

A. PST1 plus ECOR1

B. Kpn1+BamHI

C. Xmn1 alone

Mix for 7 reactions if Pst1+EcoRI. Add 5 μL of DNA solution to 15 μL of digestion mixture and incubate 2 h at 37° C. Do the same for Kpn1+BamHI and Xmn1 digestions.

Compare to expected gel patterns: Correct pattern for pTK1 digests: i) EcoRI and PstI; ii) Kpn1 and BamHI; iii) Xmn1. Correct pattern for pTK2 digests: i) EcoRI and PstI; ii) Kpn1 and BamHI; iii) Xmn1.

### Example 3B. Making pTK3 (P_{leuA}::sprA1) and pTK6 (P_{hlgA}::sprA1) and pTK4 (P_{leuA}::sprA1 Reversed)

1. Extract gDNA from a log-phase culture of BioPlx-01 using the Qiagen “All prep” kit.
2. Digest pTK1 SprA1 with Sph1 and Pst1 to drop out the cadmium-inducible promoter (P_{cad}).
3. PCR amplify the leuA regulatory region (P_{leuA}) from Bioplx-01 gDNA using PCR primers that contain the Sph1 restriction sequence upstream and Pst-1 restriction sequence downstream. (TKO1 and TKO3 Sequences below; or backups TKO2+TKO4). Verify the restriction with gel electrophoresis as previously described.

PCR mixture:

1.0 μL of gDNA from BioPlx-01 50 ng/μL
25.0 μL dI water
10.0 μL 5X HF buffer
5.0 μL 2 mM dTNP mix
4.0 μL primer TKO1 (5 pmol/μL stock)
4.0 μL TKO3 (5 pmol/μL stock)
1.0 μL phusion polymerase NEB
50.0 μL total

### Cycles:

### 98° C. for 2 min

20 cycles of: 98° C. 15 sec—64° C. 30 sec—72° C. 1 min
15 cycles of: 98° C. for 15 sec—55° C. for 30 sec—72° C. for 1 min
Hold: 4° C., indefinitely
4. PCR amplify the hlgA regulatory region (P_{hlgA}) from Bioplx-01 gDNA using PCR primers that contain the Sph1 restriction sequence upstream and Pst-1 restriction sequence downstream. (TKO9 and TKO11 or backup set TKO10 or TKO12). PCR conditions are as above for P_{leuA} except for the identity of the primers.
5. Using the Qiagen PCR cleanup kit, clean the PCR reactions and elute into 43 μL of elution buffer
6. Cut the P_{leuA} PCR product from step 3 and the P_{hlgA} PCR product from step 4 with Sph1 and Pst1. Do this by adding 5 μL of 10× CutSmart (NEB) and 1 μL each of Sph1 and Pst1 and incubating for 2 h at 37° C.
7. Digest pTK1 with Sph1/Pst1.
8. Fractionate the pTK1 Sph/Pst digest and the Sph/Pst digested P_{leuA} and P_{hlgA} on a 1.5% agarose gel and excise the ˜6000 pTK1 backbone and the P_{leuA} (390 bp) and P_{hlgA} (253 bp) fragments with a clean razor blade.
9. Divide the pTK1 backbone slice in two and combine one half with the LeuA slice and the other half with the HlgA slice. Melt together and isolate together using the Qiagen gel extraction kit. Elute each into 30 uL EB.
10. Add 3.4 μL of 10× T4 DNA ligase buffer and 1 μL of T4 DNA ligase and incubate at 16° C. for at least 1 h.
11. Follow steps in section 2.13-2.22 for electroporation, recovery, and colony plating.
12. The two ligations aim to generate P_{leuA}::sprA1 wt in the forward orientation (pTK3) and P_{hlgA}::sprA1 wt in the forward orientation (pTK6).

### Example 3C. Making pTK4 (Conduct Steps Concurrently with pTK3)

1. Extract gDNA from a log-phase culture of BioPlx-01 using gDNA isolation kit.
2. Digest pTK2 (sense sprA1) with Sph1 and Pst1 to drop out the P_{cad} (see above for digestion conditions).
3. Insert the Sph1/Pst1 digested P_{leuA} fragment from above into the Sph1/Pst1 digested pTK2 to generate P_{leuA}::sprA1 wt in the reverse orientation (pTK4). Details of the gel extraction, ligation and electroporation processes are the same as in Section 2 of cloning above.

Screening pTK3, pTK4 and pTK6:

3.1 Pick 6 colonies of each ligation for screening
3.2 Inoculate 6 colonies of pTK3 and 6 of pTK4 and 6 of pTK6 each into 3 mL LB+carbenicillin (100 μg/mL) in 14 mL culture tubes.
3.3 Incubate with agitation for 16 h (37° C., 240 rpm).
3.4 Isolate plasmid DNA using a mini prep kit and elute DNA with 40 μL elution buffer.
3.5 Digest 5 μL of each of the 18 plasmid DNAs as follows (prepare enough digestion reaction mixture for 20 reactions to account for pipetting errors):
A. Sph1 plus Pst1
B. Xmn1.
   Add 5 μL DNA to 15 μL digestion reaction mixture and incubate 2 h at 37° C. 3.6 Verify digestion with gel electrophoresis, compare to expected gel patterns for pTK3, pTK4, and pTK6.

Making pTK5 and pTK7

1. Use gDNA of BioPlx-01 prepared above.
2. PCR amplify the clfB RR (P_{clfB}) from BioPlx-01 genomic DNA using primers with a EcoRI restriction sequence upstream and BamHI restriction sequence downstream (primers: TKO5 and TKO7)
PCR Mixture (50 μL total volume)
1.0 μL of gDNA from BioPlx-01 50 ng/μL
25.0 μL dI water
10.0 μL 5×HF buffer (NEB)
5.0 μL 2 mM dTNP mix
4.0 μL primer TKO5 (5 pmol/μL stock)
4.0 μL TKO7 (5 pmol/μL stock)
1.0 μL phusion polymerase (NEB)

### Cycles:

### 98° C. for 2 min

20 cycles of: 98° C. 15 sec—64° C. 30 sec—72° C. 1 min
15 cycles of: 98° C. for 15 sec—55° C. for 30 sec—72° C. for 1 min Hold: 4° C., indefinitely
3. Use 5 μL of the PCR reactions for gel electrophoresis as previously described.
4. Using the PCR cleanup kit, clean the PCR reaction and elute with 30 μL of elution buffer.
5. Digest the P_{clfB} PCR product with BamH1 and EcoR1 and insert it into the EcoR1/BamH1 digested pTK3 backbone to generate pTK5. This plasmid will contain sprA1 regulated by P_{leuA} and the sprA1_{AS} regulated by P_{clfB}. Using the same P_{clfB} fragment, insert it into the EcoR1/BamH1 digested pTK6 to generate pTK7. This plasmid will contain sprA1 regulated by P_{hlgA} and the sprA1_{AS} regulated by P_{clfB}SprA1. Details of the gel extraction, ligation and electroporation processes are the same as in section 2 of cloning above.

Screening for pTK5 and pTK7

3.1 Inoculate 6 colonies of ligation pTK5 and 6 colonies of ligation pTK7 into 3 mL LB+carbenicillin (100 μg/mL) in 14 mL culture tubes.
3.3 Incubate with agitation for 16 h (37° C., 240 rpm)
3.3 Isolate plasmid DNA using a mini prep kit and elute DNA into 40 μL elution buffer.
3.5 Digest 5 μL of each of the 12 plasmid DNAs with:
A. BamHI+EcoRI
B. Xmn1 alone
   Prepare digestion reaction mixture with BamHI/EcoRI and Xmn1 following the manufacturer's suggestions.
   Add 5 μL of plasmid solution to 15 μL of digestion reaction mixture and incubate for 2 h at 37° C. Verify the digestion with gel electrophoresis as previously described.

### Example 3D. Constructing pTK8, pTK9 and pTK10 (P_{cad}-sma1, P_{hlgA}-sma1 and P_{leuA}-sma1 Respectively)

The sma1 gene was ordered from DNA2.0 with a Pst1 restriction site upstream and EcoR1 restriction site downstream to allow for insertion into the following:
pCN51 to make P_{cad}::sma1 resulting in pTK8
pTK6 from which sprA1 has been removed with Pst1/EcoR1 to make P_{hlgA}-sma1 resulting in pTK9
pTK3 from which sprA1 has been removed with Pst1/EcoR1 to make P_{leuA}-sma1 resulting in pTK10
   1. Digest pCN51, pTK6 and pTK3 with Pst1 and EcoR1 by sprA1. incubating each for 2 h at 37° C.
   2. Generate the sma1 fragment by digesting the ordered plasmid containing the gene with Pst1 and EcoR1 (2 h at 37° C.). Verify the digestion with gel electrophoresis (expected fragment size is 757 bp).
   3. Follow steps 2.5 to 2.22 for gel extraction, ligation, electroporation, recovery, and antibiotic selection.

Screening for pTK8, pTK9, and pTK10

3.1 Inoculate 6 colonies of ligation pTK8 and 6 colonies of ligation pTK9 and 6 colonies of ligation pTK10 into 3 mL LB+carbenicillin (100 μg/mL) in 14 mL culture tubes.
3.3 Incubate with agitation for 16 h (37° C., 240 rpm)
3.3 Isolate plasmid DNA using a mini prep kit and elute DNA into 40 μL of elution buffer
3.5 Digest 5 μL of each of the 12 plasmid DNAs with

A. Pst1 and EcoRI

B. Sph1 and Xcm1

C. Xmn1 alone

Follow previously described restriction reaction and gel electrophoresis procedures.

### Example 3E. Making pTK11, pTK12 and pTK13 (P_{cad}-rsaE, P_{hlgA}-rsaE and P_{leuA}-rsaE Respectively)

The rsaE gene was ordered from DNA2.0 with an upstream Pst1 restriction site and a downstream EcoR1 restriction site to allow for insertion into the following plasmids:
pCN51 to make P_{cad}-rsaE resulting in pTK11
pTK6 from which sprA1 has been removed with Pst1/EcoR1 restriction to make P_{hlgA}-rsaE resulting in pTK12
pTK3 from which sprA1SprA1 has been removed with Pst1/EcoR1 restriction to make P_{leuA}-rsaE resulting in pTK13
   1. Digest pCN51, pTK6 and pTK3 with Pst1 and EcoRI sprA1 as described in previous sections.
   2. Digest ordered DNA containing rsaE Pst1 and EcoR1 following manufacturer's suggestions.
   Verify digestion with gel electrophoresis (rsaE fragment should be 142 bp).
   3. Follow steps 2.5 to 2.22 for gel isolation, ligation, electroporation, recovery, and antibiotic selection.

### Example 4. Production of sprA1 Clamp and No Clamp Constructs Using DNA2.0 to Make Inserts

Here pCN51 is employed as the vector backbone because it has cadmium inducible promoter (P_{cad}), Bla terminator, ampicillin resistance for E. coli and erythromycin resistance for Staphylococcus aureus. In Drutz 1965, 502a was shown to be sensitive to 2 μg/mL erythromycin.

Plasmid pTK1: Positive control cassette to prove that sprA1, when induced, causes death.

1. Order the following insert from DNA2.0. It is cut out of the ordered vector with Pst1 and EcoR1 restriction enzymes, and inserted into Pst1/EcoR1-digested pCN51. It is just the open reading frame and a little flanking downstream to capture sprA1-essentially as in Sayed et al. 2012, except that the P_{cad} feature is used instead of the aTc promoter (Pₜₑₜ). This sequence was verified in pDRAW, to assure strategy will work.

SEQ ID NO: 122
CTGCAGggtaccgcagagaggaggtgtataaggtg
ATGCTTATTTTCGTTCACATCATAGCACCAGTCATCAGTGGCTGTGC
CATTGCGTTTTTTTCTTATTGGCTAAGTAGACGCAATACAAAATAGGTGA
CATATAGCCGCACCAATAAAAATCCCCTCACTACCGCAAATAGTGAGGGG
ATTGGTGTataagtaaatacttattttcgttgt
ggatccttgactGAATTC

Resulting plasmid: pTKXXX
Underlined upper case: start codon
Italicized: stop codon
BOLD: PstI site upstream
UPPERCASE BOLD ITALICIZED: EcoRI site
lower case bold italicized: KpnI site
Rust color: shine-delgarno (naturally used for SprA1)
Lower case underlined: BamHI site

Produce pTK2: Reverse the Insert in pTK1

1. Cut the insert of pTK1 out with Kpn1 and BamHI and insert it into Kpn1 and BamHI-digested pCN51. This creates the antisense orientation of the toxin gene and toxin should not be expressed at all, whether it is induced with cadmium or not. Product is pTK2.

PTK3 and PTK4: P_{hlgA} regulating sprA1 toxin to prove that sprA1, when induced by serum or blood, causes cell death (forward and reverse constructs, respectively).

sprA1_{AS} is present but has only its natural promoter, so the expression clamp should be inactive—and also if P_{hlgA} is leaky, some cell toxicity may occur because the expression clamp is not present.

pTK3:

1. Digest pTK1 (sense sprA1) with Sph1 and Pst1 to drop out P_{cad}.
2. PCR amplify the hlgA regulatory region (P_{hlgA}) from strain 502a using PCR primers that contain an upstream Sph1 restriction site and Pst1 downstream restriction site. (Primers: TKO1 and TKO2)
3. Cut the P_{hlgA} PCR product with Sph1 and Pst1 and insert it into the Sph1/Pst1 digested pTK1 to generate P_{hlgA}::sprA1 wt in the forward orientation generating pTK3.

| | |
|---|---|
| 1 | TTGCGAAATC CATTCCTCTT CCACTACAAG CACCATAATT AAACAACAAT |
| | AACGCTTTAG GTAAGGAGAA GGTGATGTTC GTGGTATTAA TTTGTTGTTA |
| 51 | TCAATAGAAT AAGACTTGCA AAACATAGTT ATGTCGCTAT ATAAACGCCT |
| | AGTTATCTTA TTCTGAACGT TTTGTATCAA TACAGCGATA TATTTGCGGA |
| 101 | GCGACCAATA AATCTTTTAA ACATAACATA ATGCAAAAAC ATCATTTAAC |
| | CGCTGGTTAT TTAGAAAATT TGTATTGTAT TACGTTTTTG TAGTAAATTG |
| 151 | AATGCTAAAA ATGTCTCTTC AATACATGTT GATAGTAATT AACTTTTAAC |
| | TTACGATTTT TACAGAGAAG TTATGTACAA CTATCATTAA TTGAAAATTG |
| 201 | GAACAGTTAATTCGAAAACGCTTACAAATGGATTATTATATAT SEQ ID NO: 327 |
| | CTTGTCAATT AAGCTTTTGC GAATGTTTAC CTAATAATATAT SEQ ID NO: 328 |

TKO1: 5'-gatgcGCATGCTTGC GAAATC CATTCCTCTT-3' (contains SphI)
SEQ ID NO: 329
TKO2: 5'-catgatCTGCAGATATATAATAATCCATTTGTAAGCG-3'
(contains PstI) SEQ ID NO: 20

pTK4:

1. Digest pTK2 (reverse sprA1) with Sph1 and Pst1 to drop out the cadmium promoter.
2. Insert the same Sph1/Pst1 digested P_{hlgA} PCR product. This provides the reverse orientation SprA1.

pTK5: Expression clamp for pTK3, using P_{clfB} to drive SprA1_{AS}

1. PCR amplify the P_{clfB} from 502a gDNA using with primers to generate an upstream EcoR1 restriction site and a BamHI downstream restriction site.
2. Digest pTK3 with EcoR1 and BamHI and insert the EcoR1/BamHI-digested P_{clfB}.

The resulting plasmid is called pTK5 and will contain the SprAl sense regulated by the serum responsive P_{hlgA} (upregulated) and the sprA1_{AS} SprA1 regulated by serum responsive P_{clfB} (downregulated).

The sequence below is the P_{clfB} (219 nucleotides immediately upstream of TTG start codon).

| | |
|---|---|
| 1 | AGGTGATGAA AAATTTAGAA CTTCTAAGTT TTTGAAAAGT AAAAAATTTG |
| | TCCACTACTT TTTAAATCTT GAAGATTCAA AAACTTTTCA TTTTTTAAAC |
| 51 | TAATAGTGTA AAAATAGTAT ATTGATTTTT GCTAGTTAAC AGAAAATTTT |
| | ATTATCACAT TTTTATCATA TAACTAAAAA CGATCAATTG TCTTTTAAAA |
| 101 | AAGTTATATA AATAGGAAGA AAACAAATTT TACGTAATTT TTTTCGAAAA |
| | TTCAATATAT TTATCCTTCT TTTGTTTAAA ATGCATTAAA AAAAGCTTTT |
| 151 | GCAATTGATA TAATTCTTAT TTCATTATAC AATTTAGACT AATCTAGAAA |
| | CGTTAACTAT ATTAAGAATA AAGTAATATG TTAAATCTGA TTAGATCTTT |
| 201 | TTGAAATGGA GTAATATTT SEQ ID NO: 129 |
| | AACTTTACCT CATTATAAA SEQ ID NO: 130 |

Primer: 5′--gactacGAATTC AGGTGATGAA AAATTTAGAA-3′ SEQ
ID NO: 13
Primer: 5′ cttagctGGATCCAAATATTACTCCATTTCAA-3′ SEQ ID
NO: 15
PepA1 (SA newman)
MQGFKEKHQELKKALCQIGLMRSISEVKQLNIA SEQ ID NO: 113

pTK6. serum responsive promoter 2—SprA1

In this construct, the responsive promoter 2 is P_{leuA}.

1. Digest pTK1 (containing sense sprA1) with Sph1 and Pst1 to drop out P_{cad}.
2. PCR amplify P_{leuA} from Staphylococcus aureus 502a gDNA using PCR primers that contain an upstream Sph1 restriction site and a downstream Pst1 restriction site (Primers: TKO5 and TKO6).
3. Digest the P_{leuA} PCR product with Sph1 and Pst1 and insert it into the Sph1/Pst1 digested pTK1 to generate P_{leuA}::SprA1 wt in the forward orientation generating pTK6.

In Staphylococcus aureus, the ilvleu operon consists of ilvDBHC-leuABCD-ilvA (9 genes). It is the BCAA biosynthetic operon.

### Example 5. Preparation of Electrocompetent DC10B

Electrocompetent bacteria are prepared by harvesting log-phase cells and washing the cells extensively in sterile de-ionized water to lower the conductivity and to render the cells into an appropriate osmotic state for the electroporation process.

1. From freshly streaked antibiotic free plates, inoculate 250 mL LB media with each strain and incubate with agitation (37° C., 240 rpm).

2. Turn on centrifuge and cool rotor to 4° C. well in advance of harvesting cells. Place 1 L of sterile filtered 10% glycerol on ice well in advance of harvesting cells.

3. Monitor growth by OD₆₃₀ and when the cells are at 1.0 OD₆₃₀ units per mL, place flask immediately on wet ice for 10 minutes. From this point on the cultures must be kept ice cold. Pour each 250 mL culture into chilled 500 mL sterile centrifuge bottles.

4. Centrifuge (15 mins, 3500 rpm, 4° C.). Pour off the supernatant and aspirate any residual broth.

5. Add 250 mL of sterile 10% glycerol to each of the centrifuge bottles and completely suspend the cells by pipetting up and down.

6. Repeat 4 and 5 two more times.

7. Pour off the supernatant and suspend the cells in 2 mL 10% glycerol by pipetting up and down.

8. To freeze, aliquot 100 μL of the culture to microcentrifuge tubes on wet ice. Once you have used all of the culture, transfer the tubes to a dry ice/ethanol bath for 10 minutes. Once the cultures are frozen, transfer cells to a −80° C. freezer for storage.

To confirm cell's efficiency—transform cells with 1 μL of pUC19 (10 pM).

Electroporation conditions for E. coli are 1500 V, 25 μF, 200 ohms. Use 1 μL of plasmid miniprep from DH5α and electroporate it into 50 μL of the electrocompetent DC10B.

1. Electrocompetent E. coli are thawed on ice, and 1 μl of plasmid is added to 50 μl of cells in an ice cold 0.1 cm gap electroporation cuvette.

2. Electroporate as above and add recovery medium immediately (1 mL, SOC medium).

3. Agitate at 37° C. for 1 h at 250 rpm and plate 100 μL onto LB+100 g/mL carbenicillin. Incubate plates for 16 h at 37° C.

### Example 6. SA Transformation

Techniques for transformation are adapted from Chen, W., et al. 2017, Rapid and Efficient Genome Editing in Staphylococcus aureus by Using an Engineered CRISPR/Cas9 System. J Am Chem Soc 139, 3790-3795. Materials to have on hand: LB agar plate containing 50 μg/ml kanamycin; sequencing primers for PCR screening of 12 clones; TSB broth with kanamicyn, sterile tubes for bacterial growth; PCR reagents to do colony PCR (master mix for 500 μl) and PCR grade H₂O.

10 μL product of Golden Gate assembly is transformed into 100 μL E. coli DH10B competent cells. The successful colonies are selected on a LB agar plate containing 50 μg/mL kanamycin. The success for the construction of the pCasSA-NN_spacer plasmid was verified by PCR or sequencing.

### Example 7. Purification Plasmids from E. coli DH10B to Confirm Sequence

1. DNA Sequencing of Inserts

Primers TKO13 through TKO20 are used variously to sequence the inserts of these 13 plasmids. The primers to use for each plasmid are indicated in Table 15. The kill gene inserts are obtained from DNA2.0. PCR amplified P_{leuA} and P_{hlgA} promoters to evaluate any possible polymerase errors for these fragments.

2. Assembly and Confirmation of Sequences

2.1 Raw chromatograms are inspected and only high quality regions (very high signal/noise and good peak separation) are chosen to use in assembly process.

2.2 Overlap regions of sequence reads from successive primers are identified and removed; unique reads are strung head to tail in Microsoft word with color coding of the text.

2.3 Clustal W is used to generate sequence alignments of theoretical sequences to the actual. Any discrepancies are confirmed by manual inspection of chromatograms.

### Example 8. Preparation of Electrocompetent BioPlx-01 and RN4220

Electrocompetent bacteria are prepared by harvesting log phase cells and washing the cells extensively in sterile de-ionized water to lower the conductivity and to render the cells into an appropriate osmotic state for the electroporation process.

Materials to have on Hand:

1. 500 mL orange capped v-bottom corning centrifuge bottles

2. 50 mL falcon tubes

3. 1.5 mL sterile microcentrifuge tubes

4. 96 well plate for A630 measurements

5. 10 and 25 mL sterile pipets and sterile pipet tips all sizes

6. TSB broth (need 600 mL total)

7. 1 L of Sterile 500 mM sucrose on wet ice well in advance of harvesting cells

Protocol

1. From freshly streaked antibiotic free plates, inoculate 250 mL TSB media with each strain and incubate with agitation (37° C., 250 rpm).

2. Turn on centrifuge and cool rotor to 4° C. well in advance of harvesting cells. Place 1 L of 10% glycerol on ice well in advance of harvesting cells.

3. Monitor growth by OD₆₃₀ and when the cells are at 1.0 OD₆₃₀ units per mL, place flask immediately on wet ice for 15 min. From this point on the cultures must be kept ice cold. Pour each 250 ml culture into chilled 500 ml sterile centrifuge bottles.

4. Centrifuge at 2900 rpm for 15 min. Pour off the supernatant and aspirate any residual broth.

5. Add 250 ml of 10% glycerol to each of the centrifuge bottles and completely suspend the cells by pipetting up and down.

6. Centrifuge at 2900 rpm for 15 min. Pour off the supernatant, it is not necessary to aspirate. Completely suspend the cells in 250 ml glycerol and re-centrifuge.

7. Pour off the supernatant and suspend the cells in the residual glycerol by pipetting up and down.

8. To freeze, add 100 microliters of the culture to microcentrifuge tubes on wet ice. Once you have used all of the culture, transfer the tubes to a dry ice/ethanol bath for 10 minutes. Once the cultures are frozen, transfer them to a −80° C. freezer.

### Example 9. Design and Test CRISPR gRNA Sequences and Test pCasSA Simultaneously

In this example a CRISPR-Cas system is obtained that is effective in Staphylococcus aureus (pCasSA) from Addgene (Addgene plasmid repository, Cambridge, Mass.), identify an intergenic region to target from prior experiments, and finally, design and test gRNA aimed for the intergenic region.

1. Order verified CRISPR components from Addgene as shown in Table 16.

TABLE 16
CRISPR Plasmids

| ID | Plasmid | Gene/Insert | Vector Type |
|---|---|---|---|
| 42876 | pCas9 | tracr/Cas9 | Bacterial Expression, CRISPR; |
| | | | E. coli |
| 42875 | pCRISPR | CRISPR-BsaI | Bacterial Expression, CRISPR; |
| | | | E. coli |
| 65770 | BPK2101 | CRISPR-Cas9 | Bacterial expression plasmid for |
| | | | Staphylococcus aureus Cas9 & |
| | | | sgRNA (need to clone in spacer |
| | | | into BsaI sites): T7- |
| | | | humanSaCas9-NLS-3xFLAG-T7- |
| | | | BsaIcassette-Sa-sgRNA |
| 98211 | pCasSA | CRISPR-Cas9 | Sa-specific CRISPR |

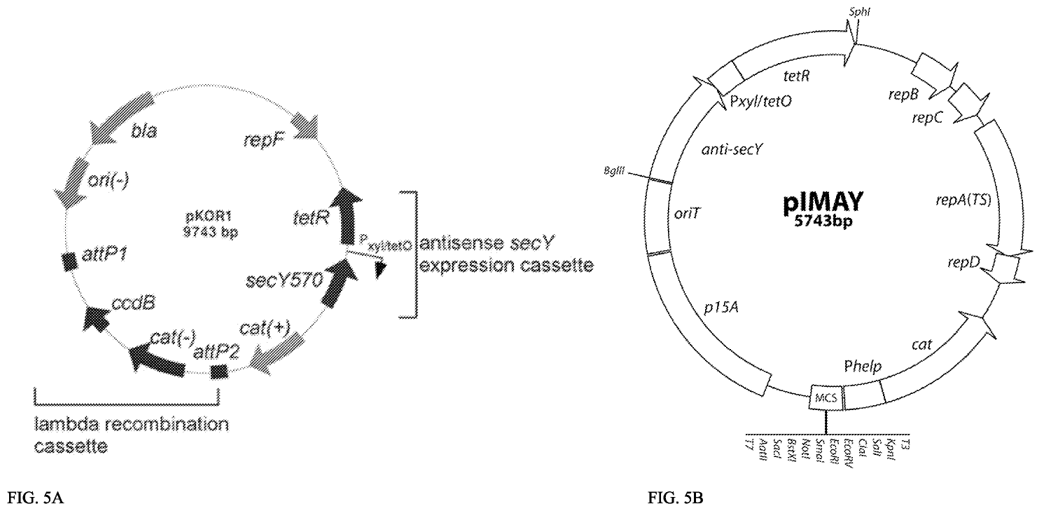

2. Select CRISPR gRNA target sites. Find where to target, this should be in an intergenic region so as not to disrupt viability. Currently, one such region has been identified between 1,102,100 and 1,102,700 bp in the 502a genome, GenBank: CP007454.1, as shown in FIG. 8. This region aligns with the region previously identified in the recombinant approach.

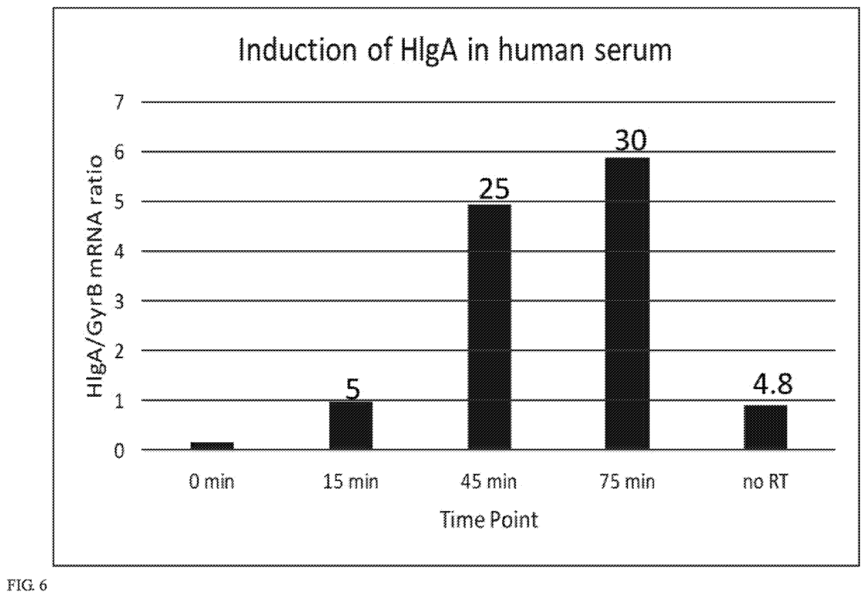

3. Once region has been chosen, use CRISPRScan (http://www.crisprscan.org/) Moreno-Mateos et al., 2012, Nature Methods 12, 982-988, to find putative gRNAs as shown in FIG. 9; note that the usable sequence is in all caps.

4. Check for possible off-target binding using BLAST (https://blast.ncbi.nlm.nih.gov/Blast.cgi?PAGE_TYPE=BlastSearch&PROG_DEF=bla stn&BLAST_PROG_DEF=megaBlast&BLAST_SPEC=MicrobialGenomes_1280&DB_GROUP=AllMG) or searching the sequence directly (APE or similar). Note: gRNA marked as non-canonical will often have a single mismatched base pair, these will likely still work but may cause additional off target effects

5. Modify and order oligos as shown in Table 4B, FIG. 4A-4D. Name of oligos is shown in the format=oligo #, BPC (BioPlx CRISPR), Target #, direction (FOR or REV), followed by the target sequence.

6. Add each of the CRISPR targeting sequences into the pCasSA plasmid as per protocol shown below, adapted from Chen, W. et al. 2017. Rapid and Efficient Genome Editing in Staphylococcus aureus by Using an Engineered CRISPR Cas9 System. J Am Chem Soc 139, 3790-3795.

a. Oligo Design

Select a 20 bp-spacer sequence before NGG (NGG is not included in the spacer) in the target gene of Staphylococcus aureus (40%˜60% GC ratio is the best). Synthesize the two oligos in the following form (described above):

Note: FOR primer should be immediately upstream of the NGG in the target sequence.

5′-GAAANNNNNNNNNNNNNNNNNNNNN-3′
3′-NNNNNNNNNNNNNNNNNNNNNCAAA-5′

b. Phosphorylation

Prepare phosphorylation mixture as shown in Table 17.

TABLE 17
Phosphorylation mixture

| | | |
|---|---|---|
| 2 | μl | oligo I (50 μM) [symbol] |
| 2 | μl | oligo II (50 μM) [symbol] |
| 5 | μl | 10x T4 DNA ligase buffer (NEB) |
| 1 | μl | T4 polynucleotide kinase (Takara) |
| 40 | μl | ddH2O |
| 50 | μl | total |

Incubate at 37° C. for 1 hour.

c. Annealing

Add 2.5 μl of 1 M NaCl to the phosphorylated oligo pairs. Incubate at 95° C. for 3 min and slowly cool down to room temperature (use a thermocycler). (Alternatively, use a heat block and take the block out of the heater and let it cool naturally for 2 hours.) Dilute the annealed oligos 20 times using ddH2O.

d. Vector Digestion

Digest 1-2 ug of pCas9 with BsaI (NEB) as shown I Table 18.

TABLE 18
Vector digestion mixture

| | |
|---|---|
| x ul (1-2 ug) | pCas9 |

| | | |
|---|---|---|
| 1 | ul | BsaI (NEB) |
| 5 | ul | 10 x NEB Buffer |
| 0.5 | ul | 100X BSA |

| | |
|---|---|
| y ul (to 50 ul) | ddH₂O |

| | | |
|---|---|---|
| 50 | ul | total |

Gel purify digested pCas9 (important for successful cloning).

e. Ligation

Prepare ligation mixture as shown in Table 19.

TABLE 19
Ligation mixture

| | |
|---|---|
| 1 ul (possibly more) | Gel purified, BsaI digested Cas9 |
| 2 ul | Diluted oligos |
| 2 ul | 10x T4 ligase buffer |
| 1 ul | T4 ligase |
| x ul (to 20 ul) | ddH₂O |
| 20 ul | total |

Incubate at RT for 2 h or 16 C for O/N.

Transform into E. coli cells (DH5a, DH10B or DC10B).

f. Select for Plasmid Uptake

Select for plasmid uptake by plating cells on LB-agar plates with Kanamycin (50 ug/mL). Note: The pCasSA plasmid causes the E. coli to grow very slowly at 30° C. and plates may need to be incubated for 24-36 hours in order to see colonies.

Once colonies are visible select a few for liquid grow up in LB broth with Kanamycin (50 ug/mL). Save an aliquot of liquid culture for easier grow up at a later date.

In a cryotube, add 50% sterile glycerol to liquid culture mix by inverting, then place at −80° C. for long term storage.

Extract the plasmids using Qiagen kit, spec and store and −20° C.

7. Verification of Inclusion by PCR and/or Sequencing

a. PCR Testing

Test using 21BPC FOR (SEQ ID NO: 63) and 22BPC REV (SEQ ID NO: 64) on the templates generated above in step 6.

Perform PCR of constructs. The PCR products will be ˜275 bp in the uncut pCasSA vector (positive control=intact pCasSA vector). PCR using the digested pCasSA vector should not produce any products (negative control=Bsa1 digested pCasSA vector).

A small portion of the digested product should be tested to ensure 100% efficacy. Testing can be by PCR or gel electrophoresis directly on the digested plasmid. PCR on the pCasSA vector with the gRNA sequences will produce ˜278 bp amplicons. Note: these will not be visibly different when compared to the intact pCasSA vector. As such, the Bsa1 digestion needs to be 100%.

b. Sequencing Method

Prepare the PCR products generated above for sequencing. Clean up PCR reaction using spin column clean up kit per manufacturers protocol.

Measure concentration of purified PCR product using NanoDrop.

Mix sample with either forward or reverse primer (21BPC FOR and 22BPC REV, respectively) for sequencing with Quintara Biosciences.

PCR product at 5 ng/ul and primer at 5 pmol/ul (5 uM). PCR products from the intact pCasSA vector should be sequenced alongside the other products to provide a baseline.

8. Testing CRISPR-Cas Efficacy/Targeting

Introduction of any plasmid with the inserted gRNA sequences should cause a double-strand break at the targeted CRISPR site. Additionally, the lack of a homologous sequence for homology directed repair (HDR) will cause double strand break induced lethality. Therefore, transforming the targeting plasmids with the targeted plasmid should result in a death rate corresponding to the CRISPR targeting efficacy.

Transform each of the 10 (assuming all targeting combinations worked) into separate aliquots of electrocompetent RN4220 Staphylococcus aureus cells.

In this case targets 1, 4, and 6-10 should show activity in the RN4220 cells (the sequences are similar enough to allow CRISPR gRNA binding).

### Example 10. Design and Test Homology Dependent Repair Templates and Efficacy Using a Fluorescent Reporter Controlled by a Constitutive Promoter

a. Homologous arms are designed of varying length (200, 300 and 400 bp) corresponding to the ˜600 bp intergenic region identified above. For proof of viability, a fluorescent reporter gene (e.g., mCherry) is inserted under control of a constitutive promoter (rspL). The promoter and reporter will be flanked with restriction sites (Not1 and Xma1) to allow transgene swapping. The current design contains a single stop codon. Optionally additional stop codons may be added. Constructs are designed and ordered through ATUM (formerly DNA2.0). This entire sequence (homologous arms+promoter+mCherry) is placed into the pCasSA vector using the Xhol and Xbal restriction sites.

b. Checking for mCherry Incorporation/Expression

Once the full pCasSA-XX-XXX vector is assembled and transformed into an Staphylococcus aureus strain, verify: 1) mCherry expression, and 2) genomic incorporation of the mCherry sequence. We currently have a few viable methods to check for these. Note: mCherry expression should occur in bacteria that maintain the plasmid as well as those with successful incorporation. To differentiate these, the plasmid must be cured (removed), except in the case of PCR which may be able to differentiate between the two.

For Plasmid curing (with repF cassette):

Grow a liquid culture at 30° C. with antibiotic as previous;

Dilute 3-5 ul of this culture 1000-fold in fresh TSB (no antibiotic);

Place at 42-43° C. until growth is apparent (e.g., overnight).

Streak the liquid culture on TSA plates with and without chloramphenicol and grow at 37° C.

Cultures should grow on −chlor plate and should not on +chlor plate at 37° C., if so, the plasmid has been removed

For Fluorescence Microscopy:

The mCherry fluorophore is excited by ˜587 nm light and emits ˜610 nm.

For PCR:

PCR across the inserted region to confirm incorporation. Primers designed to amplify: Across the insertion region (41/42 and 43/44). To test for the presence of mCherry (51/45). To verify the presence of genomic DNA (TKO 1/3). Mixing and matching insertion and mCherry primers can also serve to test for mCherry incorporation.

Incorporation may also be confirmed by Western blot analysis.

Employ western blot equipment: gel box and iBlot transfer system. Employ Primary anti-mCherry antibody, Secondary colorimetric antibody, Precast gels (or gel casting equipment and reagents), iBlot transfer kits, Protease inhibitors, Protein extraction solutions (e.g., RIPA), Protein markers (ladders), and Buffers (TBS, tween etc.) as known in the art.

### Example 11. Analysis of KS Promoters with Fluorescent Reporters

The fluorescent reporter is under control of the promoters identified in the recombinant approach (PleuA, PhlgA etc). This combination allows testing of the efficacy of the chosen promoter with a measurable (positive) outcome. Preferably, the mCherry would be placed in the constructs based on the pCN51 backbone. The combination is used to test for multiple possible issues:

If the plasmid containing cells are exposed to blood/serum mCherry should be expressed. This can be verified either with fluorescence microscopy (Ex 587 nm, Em 610 nm) or by western blotting for the mCherry protein.

If the mCherry protein is created in “normal” conditions (no blood/serum activation) then the promoter is “leaky”. Leaky activation could explain some of the issues obtaining KS plasmids with certain promoters (i.e. P_{leuA}) as even low levels of KS expression could cause a loss of viability.

What is the rate and conformity of the upregulation caused by a specific primer?

Cells are viewed in real time (fluorescence microscopy) or through time course sampling (western blot) to observe the rate of fluorescence generation upon exposure to blood and/or serum.

### Example 12. Insertion of KS into BioPlx-01, Verify Incorporation, Test for Efficacy and Longevity

A KS of choice is inserted in a pCasSA vector using Not1 and Xma1 restriction sites flanking each sequence. The pTK is amplified using primer BP-40 which adds the Xma1 restriction site. The KS is inserted into Staphylococcus aureus 502a cells and genomic incorporation verified. The incorporated cells are cured of the plasmid and tested for KS activity when exposed to blood/serum. The KS cells named “BioPlx-XX” are then passaged as described herein to analyze longevity and viability.

### Example 13. Confirm/Characterize the Rate and Extent of Serum-Induced Cell Death

The KS cells BioPlx-XX having the KS are grown side-by-side with BioPlx-01 (Staphylococcus aureus 502a WT) in TSB, and then washed and shifted to fresh human serum. The KS strain will “flatline” soon after the shift whereas the WT strain will begin to grow in the serum.

### Example 14. Evaluate the Stability of the KS Strain BioPlx-XX

This experiment is performed to demonstrate that the KS in BioPlx-XX is phenotypically and genotypically stable during in vitro propagation.

Phenotypic stability (in this case, KS performance) will be assessed by determining the rate of cell death in serum after passaging the strain for X, Y and Z generations, where X is the number of doublings experienced in strain manufacturing to produce a single clinical lot of material sufficient to treat 200 patients, and Y and Z are the number of generations experienced after up to 41 total culture doublings. We are aiming for 4×10⁹ cells per patient X 200 patients=8×10¹¹ total cells.

A dose of 4×10⁹ cells per patient X 200 patients=8×10¹¹ total cells.

1. Inoculate 5 mL of TSB with a single large colony of BioPlx-01 and a second 5 mL of BioPlx-02—both have been streaked from the frozen master cell banks. (approximate density is 0.05 A630/mL).

2. Allow the 2 strains to grow to 1.6 A630 units per mL (monitor in the Biotek plate reader; 5 doublings) This is ˜mid exponential phase. (remember that the linear range of the instrument is between 0.1 and 0.9— you must dilute samples in TSB to stay in this linear range). Keep detailed notes on growth rates. We are assuming for the sake of this calculation that about 4 A630 units/mL=8×10⁹ CFU/mL. Volume of saturated culture needed to obtain 8×10¹¹ CFU total=(8×10¹¹ CFU/8×10⁹ CFU/mL)=100 mL

3. Use the starter cultures from (2) to inoculate 100 mL “final” cultures of each to a density of 0.05 A630 units per mL. 1.6 mL starter is added to 98.4 mL TSB.

4. Allow the two strains to grow at 37 C/250 rpm. Monitor the density until an A630 of 3.2 is reached. (6 doublings). Create a new culture of each strain-100 mL initiated at 0.05 A630 units/mL (this is “round 2”). Return the flasks to the shaker 250 rpm/37 C.

5. Harvest a 1 mL volume of cells from step 4 into 50 mL PBS for each strain.

5A. Snap freeze a second 1 mL of culture and place at −80 C for later genetic tests (see genotypic stability below).

6. Centrifuge 2900 rpm for 15 min.

7. Aspirate the supernatant and vortex the cell pellet to resuspend.

8. Bring volume again to 50 mL in PBS and harvest as in step 6.

9. Resuspend the pellets of each strain (BioPlx-01, BioPlx-02) in pre-warmed fresh human serum 20 ml each.

10. Shake at 250 rpm/37 C, monitoring growth. Expected outcome is that BioPlx-01 grows and BioPlx-02 (KS) does not. Collect enough data-points that the slopes of each can be calculated from semilog plots and ratioed. This ratio will be a measure of KS performance. Kill ratio (KR)=slope of BioPlx-01 growth in serum/slope of BioPlx-02 growth in serum. This KR is a measure of KS performance at 11 total doublings was reached in TSB.

11. The “round 2” culture from step 4A will be monitored until an A630 of 3.2 is reached (6 doublings). Use this to seed a “round 3” culture to 0.05 A630/mL, then follow steps 5-10 using the Round 2 saturated culture. The KR is a measure of KS performance at 17 total doublings.

12. The “round 3” culture from step 11 will be monitored until an A630 of 3.2 is reached (6 doublings), then split back again to 0.05 A630/mL. This process of growth to 3.2 followed by splitting to 0.05 was performed 4 times as follows: Round 3: was 23 doublings; Round 4: 29 doublings; Round 5: 35 doublings; Round 6: 41 doublings. Follow steps 5-10. The KR is a measure of KS performance at 41 doublings.

Plot KR as a function of culture doubling #.

Genotypic Stability:

1. Find the samples of BioPlx-02 cells from each time point 11, 17 and 41 doublings, see step 5A.

2. Conduct NextGen sequencing to determine the sequence homogeneity of this sample. Single molecule sequencing may be used to determine the % of mutations occurring in a population of cells at a given time point.

### Example 15. Candidate Serum and Blood Responsive Promoters Screened by Fluorescence to Detect Up-Regulation

Overview. In this example, potential Staphylococcus aureus promoters were tested for activity in blood and/or serum. Candidate promoters were selected from the literature based on the upregulation of gene expression after exposure to blood or serum. These promoters were then cloned upstream of a reporter molecule, green fluorescent protein (GFP), which fluoresces when the promoter is activated. After several growth steps, Staphylococcus aureus cells containing this promoter-GFP cassette were exposed to blood or serum, and the activity of GFP was viewed with fluorescent microscopy. The results of this screen show several promoters with varying degrees of activity in blood and/or serum, which may be used to regulate a molecular modification such as a kill switch, virulence block or nanofactory.

In example 1, a non-pathogenic strain of Staphylococcus aureus, denoted 502a, was used to exclude methicillin-resistant Staphylococcus aureus (MRSA) from the human skin microbiome. While the application of 502a has shown no adverse side effects in this trial, a kill switch was designed as an additional measure of safety. The kill switch molecular modification disclosed herein may be incorporated to target microorganisms such as Staphylococcus aureus 502a or RN4220 cells, and will function to inhibit cell growth, either by slowing cell growth, or promoting cell death, upon exposure to blood or serum. As such, the possibility of systemic infection in patients will be reduced or eliminated. The kill switch comprises two key elements a kill gene to slow or stop cell growth, and a blood or serum responsive promoter to control the kill gene expression. In this example, candidate Staphylococcus aureus promoters were tested for increased activity in blood or serum. Candidate promoter sequences derived from Staphylococcus aureus strain 502a genome (NCBI CP007454.1), including about 300 bp upstream and including start codon are shown in Table 20.

Table 20. Candidate Promoter Sequences

TABLE 20
Candidate Promoter Sequences

| Gene/ | |
|---|---|
| Description | Nucleotide sequence |
| leuA | Atttttagacaattctaactattaaagtgatatataccattcacggaaggagtataataaaatgcttaatcaatatac |
| | tgaacatcaaccgacaacttcaaatattattattttattatactctttaggactcgaacgttagtaaatatttactaaac |
| | gattaagtcctatttctgtttgaatgggacttgtaaacgtcccaataatattgggacgtttttttatgttttatctttcaat |
| | tacttatttttattactataaaacatgattaatcattaaaatttacgggggaatttactatg |
| | (SEQ ID NO: 132) |
| hlgA2 | Acttcaaattttcacaaactattgcgaaatccattcctcttccactacaagcaccataattaaacaacaattcaata |
| | gaataagacttgcaaaacatagttatgtcgctatataaacgcctgcgaccaataaatcttttaaacataacataatg |
| | caaaaacatcatttaacaatgctaaaaatgtctcttcaatacatgttgatagtaattaacttttaacgaacagttaatt |
| | cgaaaacgcttacaaatggattattatatatatgaacttaaaattaaatagaaagaaagtgatttctatg |
| | (SEQ ID NO: 133) |
| hrtAB | Gttcatattgagttcatatttcaaccttatactgacgctaaagaagaaatagggagaagtgaatcgatatg |
| | (SEQ ID NO: 134) |
| hlb | Ttcaggctatcaataatgctttgaaatcagcctgtagagtcaataatataccaattattacatcgcacgcattaaga |
| | cac (SEQ ID NO: 135) |
| sbnC | Actcattgttcttatttactagcaaaaggtgtatctatacattacatttctaaaagattaggtcataaaaatatagcaa |
| | t (SEQ ID NO: 136) |
| isdI | Aactacatccgtgtattcgcatttgttagaagaaaaatttaatgaagaggacaaaaaaacaactaaaattttagaa |
| | agta (SEQ ID NO: 137) |
| isdG | Tgtaatttagggacccattagggactccaaacccaataaatactgttgttacaaggtttctatg |
| | (SEQ ID NO: 138) |
| sbnE | Gaatacttcaaggattaacatatagtgcattgattcaaagtgtcatgtttgttgtcgtgaatgcgtgtcatcaacaa |
| | cttaaaggcacatttgttggaacgacgaacagtatgttagttgttggtcaaattattggcagtcttagtggcgctgc |
| | cattacaagttatactacaccagctactacgtttatcgttatgggcgtagtatttgcagtaagtagtttatttttaatttg |
| | ttcaaccatcactaatcaaatcaacgatcacacattaatgaaattatgggagttgaaacaaaaaagtg |
| | (SEQ ID NO: 139) |
| lrgA | Atgaaaaacgattgaatcccacttattttatacgtattcatcgttcatatattattaacacgaaacacattaaagaag |
| | tgcaacaatggtttaactacacttatatggtaatattgacaaatggtgtcaagatgcaagttggacgttcatttatga |
| | aagattttaaagcgtcgataggattactttaacagtaatccttttttttatgcattttacctatgatattttgtatttcgga |
| | ctaaaaatcacgcaaatcgaagtgagccatctatactttagttaaatcaaacgtaggaggcaatg |
| | (SEQ ID NO: 140) |
| lrgB | Gtttagtattattatttgtattattatgtactggtgctgttaagttaggcgaagtcgaaaaagtaggaacgacactaa |
| | caaataacattggcttactcttcgtaccagccggtatctcagttgttaactctttaggtgtcattagccaagcaccat |
| | ttttaatcattggactaataatcgtctcaacaatactattacttatttgtactggctatgtcacacaaattattatgaaag |
| | ttacttcgagatctaaaggtgacaaagtcacaaaaaagatcaaaatagaggaggcacaagctcatg |
| | (SEQ ID NO: 141) |
| hlgB | Aagatcctagagattatttcgttccagacagtgagttacctcctcttgtacaaagtggatttaacccttcatttatcg |
| | ccacagtatctcatgaaaaaggttcaagcgatacaagcgaatttgaaattacttacggaagaaacatggatgtca |
| | ctcatgccattaaaagatcaacgcattatggcaacagttatttagacggacatagagtccataatgcattcgtaaa |
| | tagaaactatactgttaaatacgaggtcaattggaagactcatgaaatcaaggtgaaaggacagaattgatatg |
| | (SEQ ID NO: 142) |
| fhuB | Tcaaaatgtaacaatgatcagaggcatatgtttaattattgctatgattctagcaggtattgcagttgctatcgctg |
| | gacaagttgcatttgtaggtttgatggtacctcatatagcaagatttttaattggaactgattatgctaaaattctacc |
| | attaacagccttgttaggtgggatactcgtgcttgttgccgatgtgatagcacgatatttaggagaagcgcctgtt |
| | ggtgcaatcatttcatttatcggtgttccttactttttatatttagttaaaaaaggaggacgctcaatatg |
| | (SEQ ID NO: 143) |
| splF | Gttcacctatattaaatagtaagcgagaagcaattggtgttatgtatgctagtgataaaccaacaggtgaaagta |
| | caaggtcatttgctgtttatttctctcctgaaattaagaaatttattgcagataatttagataaataaatcatccatccat |
| | acattgataaatgatttttagaaattaacaacaaaatcaacaattttaaacatctctgtgattctatttattcgaaatga |
| | tttaaaaaataaaacttcaaaaacctaaccttatatttatacgaatacttagaggagcacaaaaatg |
| | (SEQ ID NO: 144) |
| SAUS | Gatgatgtatgtttcgaatttatcaattaacatgtgaggacctcccgaggaatacatggcattaaatacacgtttaa |
| A300_ | tatttataaaggtgacttaattttgttcaagttgattttaccacgctttttttctttattcactaagacttttgaatgaagttt |
| 2268 | aaaataattgtttatcagtgataaaatatttgcaataagaagagaatggctaaataatcttaattttcagaaaagtaa |
| | ttgtaaccttactggtcttatggtaatatttttcaatattatcgacgaggatgtgttaacaatg |
| | (SEQ ID NO: 145) |
| SAUS | Ctatcattataatgagataatgtcatttttaattgagctaaacagacagggaaagacgattattatgattacgcatg |
| A300_ | atatgcatttattgtctgagtatagttcaagaacagttgtattatcaaaaggacaagtcgttgctgataccacgcca |
| 2616 | gtattgatattaaatgataaaaaaatctgtgagattgcatcattgagacaaacatcgctatttgaaatggccgaata |
| | tatagggattagcgagccacagaaattagtacaattatttattaaccatgataggaaggtgagacgccaatg |
| | (SEQ ID NO: 146) |
| SAUS | Caggcctattttctaggaaatcgatgatttattttaatatcggtcaaattattgcgaatattatttgctgggcacttatt |
| A300_ | gcaccaacattagatattttgatttataacgaaccggctaacaaggtttatacacaaggtgttatctctgcagtatta |
| 2617 | aatattatttcagttggtattattgggacaatattattaaaagcatatgcttcatctcaaataaaaaaaggtagtttac |
| | gtaaagaataatcattttgttgaatcagatatgtaaatgaatgtagaaaggtaatgatatatcatg |
| | (SEQ ID NO: 147) |
| isdA | CTATCTGCGGCATTTGCAGAATTACTGAATGTCGCGATGATGATAA |
| | TTAACGCTAAAATCGTTGTATTAAAAACTTTTAAAATATTTTTCAA |
| | AACATAATCCTCCTTTTTATGATTGCTTTTAAGTCTTTAGTAAAATC |
| | ATAAATAATAATGATTATCATTGTCAATATTTATTTTATAATCAATT |
| | TATTATTGTTATACGGAAATAGATGTGCTAGTATAATTGATAACCA |
| | TTATCAATTGCAATGGTTAATCATCTCATATAACAACACATAATTT |
| | GTATCCTTAGGAGGAAAACAACATG |
| | (SEQ ID NO: 148) |
| isdB | CTTCAGTTGATAACTTTATTAGCACAGTTGCCTTCGCAACACTTGC |
| | CCTTTTAGGTTCATTATCTTTATTACTTTTCAAAAGAAAAGAATCTA |
| | AATAAATCATCGTCACACTCATAACTTAATATATTTTTTATTTTAAA |
| | TTTTATTTAACCTATGTCATAGATATTTCATAATCTATAACATAGGT |
| | TATTTTTTTATAAAATAATGTTGCAATTAACTACCATTTCAATGTAC |
| | AATACAAGTAATCAATTGATAATGATTATCAGTTGATAATATACAA |
| | TTAGGAGTTGTTTCTACAACATG |
| | (SEQ ID NO: 149) |
| fhuA/C | Ctttcttgcagatgaataaataaatggtatgagcacacatacttaaatagaagtccacggacaagtttttgaactat |
| | gaagacttatctgtgggcgttttttattttataaaagtaatatacaagacatgacaaatcgagctatccaatttaaaa |
| | agtaatgttagtcaataagattgaaaaatgttataatgatgttcatgataatcattatcaattgggatgcctttgaaaa |
| | ttgataatttaaaaatagaaattatifittataaacagaaagaattttattgaaagtagggaaattatg |
| | (SEQ ID NO: 150) |
| ear | Tgacacctgctaattcaaacattatttgagacattcttttcaaattaattataaatttttacctatagactagtttgatatt |
| | tatctacatctcaaaattctcatcaacaatctttcacatccaacatttttactttagtttttataattcaaaacaacaaaa |
| | cgatgttaaaaaattattctattttttagttaatagatagttaatacatttttgatatttagttaattgttcttttaaaaaaat |
| | attattatattttcattgtaaacgtttacaatataaaaaaaggagcaattaaaatg |
| | (SEQ ID NO: 151) |
| fnb | Tgtacaggcgataattatgaaacacttagtatattgttttaaattagataatgatgaatttaatttgaaaaataagtat |
| | aaaaaatacaagccttgtgtgacaagggtttatgatgacttgaatacaatttataggtatatttcaaataataaaatt |
| | atcaattaacataaaattaatgacaatcttaacttttcattaactcgcttttttgtattgcttttaaaaaccgaacaatat |
| | agacttgcatttattaagtttaaaaaaattaatgaattttgcatttaaagggagatattatagtg |
| | (SEQ ID NO: 152) |
| splD | Attttaaattttgatgcatacattgaacccgggaattcaggatcaccagttctaaattctaacaatgaggtcatagg |
| | tgtggtgtatggcggtattggaaaaattggttctgaatataatggtgccgtatactttacgcctcaaatcaaagattt |
| | tattcaaaagcacattgaacaataaacaaatttaaatatacaccatgagcatgtgttcaataattttaatgaaaaac |
| | atcggtcgaatataacataaaaaaacgtctatatcaaaagcatcatgaataaacagaggagcacaaaaatg |
| | (SEQ ID NO: 153) |
| dps | Ataatagaaatagaatgtggaaaacaacatggcaccaaccaaatgattatgaaaaatcgttctttttagatgata |
| | atgcgaaagtaaaacttactgattgataaaacatacttgctaattgataatggatatactagatgatgaattaaaatt |
| | tagacatttaaaaagcggaacaccttacatttagattagaataattataaaaaagagagtaaaaacactttacaga |
| | ttagaatcattataatataataattaatataaacaagcaagacgtagacaattttaaggagtgtattaaatatg |
| | (SEQ ID NO: 154) |
| CH52_ | GAATTCTTTATAGCGCGTGCAATCACACCACAAGATAAAAGATTA |
| 00360 | AAAAGTGACAAAGCATTTATTGCATTTTTAGAAGAAACCTTCGATC |
| | AGTTCTTACCATTTTATTCTGCATAAATAACTTTGTTTAAATAATAG |
| | AGCACGTAATCACATCCATGATTTCGTGCTCTTTTTTCTTAATATTA |
| | AATCGAACGTTCAACATAATAATTCATACTTTTAAAAAAATTAAAA |
| | TAAATTTAGGTTGACCTAAACATTTTATTAGGTTATTATATTGTCCA |
| | TAAGAAGTAGAGGTGAGTCAAA |
| | (SEQ ID NO: 155) |
| CH52_ | CATAATCCCCCTCCTTAAATTTGTTCATATAAGATTATGATATCTTA |
| 00305 | GATTGCATAAAAAGACTAGGTTTAATAAAATTAAAATGTGACAAA |
| | TTAACGACAAGAGAAAATGTCAATTTTGTGACACAAATAACATTT |
| | AATTTATTGCTATAATGTATATGTTAGAAAATTTTAATAAGTAGAA |
| | TCATGCATCTAAAAGAGATTAATATTTAAGCTTCAAATTTGAGTAA |
| | ACGTGGATTACATAATTATCCCAATAAAAAAATCATTACGATTAA |
| | GTTCTTTTTATGTCGTCCACATACAATAC |
| | (SEQ ID NO: 156) |
| CH52_ | CATTTTATATTCCCTCCGTAAAATATAAAGTTTTCTTAACTAGTTTA |
| 01670 | TAATAATTTTAATTTGTAGTCAAAAAGACTTTGTAATAATGCGTTC |
| | AGTTAATTATAACTTACTTATACCTTAATATAAACAACTTAAACCC |
| | TTTTTATTATTTTTAATAACTCTAAAGTACAACTCTAATCCGCTCTC |
| | TTTAAAAATATAAATGATAATAAGTGCACATAATTTCTCAATGGAT |
| | TTTATGAATTTAAAATATGTTATCATTTCACTAGGACATTTGTAAT |
| | ATGGTATGATGCTATTTATGATTTT |
| | (SEQ ID NO: 157) |
| srtB | CATAAAAATCCTCTTTTATTAACGACGTTTCTTCAGTCATCACTAA |
| | ACCAGTTGTTGTACCGTTTTAGATTCGATTTCGTTGACTTTGACAA |
| | ATTAAGTAAATTAGCATTGGACCACCGACAATCATTAAAATAGCA |
| | TTGGCTGGAATTTCTAAAGGAGGCTGTATCACTCGTCCTAATAAAT |
| | CAGCCACTAACAATAGCCATGCACCAATAACTGTAGAAAACGGAA |
| | TAAGTACTCTGTAATTGCCCCCAACTAGCTTTCTAACCACATGTGG |
| | CACAATAATACCTAAAAAGGCTAGTTGT |
| | (SEQ ID NO: 158) |
| sbnA | CAAAAGCGCTTCCTCCTCAAATTTAAAATTCTATAATATTGTGTGT |
| | TAC CTAATTGATAATGATTCTCACTATCAAGTAATTAGGATTATAT |
| | TTTTTATGCATTTATATGTCAAATAATTATAAGTTGCATGTAAATC |
| | ATAAATATTTTATTGACTTAGGAAAAAATTTAATTCATACTAAATC |
| | GTGATAATGATTCTCATTGTCATACATCACGAAGGAGGCTAATTAG |
| | TCAATGAATAAAGTAATTAAAATGCTTGTTGTTACGCTTGCTTTCC |
| | TAC TTGTTTTAGCAGGATGTAGTGGGA |
| | (SEQ ID NO: 159) |
| clfA | CATTTTATTCCCTCTTTTTAAAAAGTCATTTTATATTAACTATATAC |
| | CCTTTAAAGATATATTTAATCTCTGTTAATGGAATTATACACTAAA |
| | ATTGCATTATAGCAATTAATTTGTATCGATATTTTATTATCCACAAT |
| | AATACTTTACTAACAAACATTTTATTTATTGCTATTTTAAGAATTAC |
| | AAACGACAACGTACGATTTGATTGCAAACATTTTTTATTATTAATA |
| | TGAACTCTACCTAATGTAATCCTAGCTTTAAATCATATTTTTTCAAA |
| | AGCAGATGTGTAATTTATGGTAC |
| | (SEQ ID NO: 160) |
| emp | CATCTGTTATTTCTCCTTTATATAGACTCAATATTATAACCAATATA |
| homolog | ATTTCCCTGTTATATTCACTAACAGCATTATATACCAGAATTTTCA |
| | GTATAATAATTAACTTGAAGTAAACGTTGTCTTAACATTTTTATTG |
| | TTTTTCAGCTTAAAATTAATTATTGATATTGATAGTTAAGCATAAT |
| | AATTTTTTCGTAATATAAAGTGAAAAAAGTAATAGTCCACACCTGT |
| | TTAGAATGTGGACTATACTAGATTGCATCATTGAAATGATGACTTT |
| | GATATTATTTATTGCTAGTTTAAAAT |
| | (SEQ ID NO: 161) |
| rsaC | CACGCTGTGTTTTAATGAAGTAAGATGAATTGATGTTGATGCAACC |
| | TAAAATATTGGTATCTCCAATATTTTAGGCTACACATCAACATAAC |
| | AAAGTCGAAGGCTAATAGTCCCATATCGTGCGTTAAATATATATTA |
| | CCCTCCTATTAATATATATACCGTTCCCGATCGCACGATATGGTGG |
| | TATTAGAACTTCTCTTTGAACGAAAGAGAAAAGCTAGAACTTATG |
| | CAGTTTTAATTAAACTGTAAACATTTGTCACTCTTTAAATCAAAGA |
| | GTAAAGTT |
| | (SEQ ID NO: 162) |
| hlgA1 | Aacaatttgtattttacaaacattaattaaaaataaaagcaagacattcgtgcaatcggttaccttaaattgtttaca |
| | actgtcaacaataccaaggttttattaactatatttacacaaaattagatttagcattccaaacaaaaaaggttaaa |
| | ttgaacggaattatggcatttttaacttaattgtaaaaaagttgataatggtcaattgttaatgaacagttaattataat |
| | aacgtccaaaatatattattatttaattaagttaaataaaattatagaaagaaagtgaaacttatg |
| | (SEQ ID NO: 163) |

Initially, 21 promoter candidates were selected from literature reporting gene expression changes when Staphylococcus aureus cells were cultured with blood or serum. The following genes are described by Malachowa N., et al. (2011). Global changes in Staphylococcus aureus gene expression in human blood. PLOS ONE 6:e18617. 10.1371/journal.pone.0018617: isdA, isdB, isdG, isdI, sbnC, sbnE, fhuA, fhuB, SAUSA300_2268, SAUSA300_2616, SAUSA300_2617, hlgB, lrgA, lrgB, ear, splD, and splF. The following genes are described by Palazzolo-Ballance A. M. et al. (2008). Neutrophil microbicides induce a pathogen survival response in community-associated methicillin-resistant Staphylococcus aureus. J Immunol 180(1):500-509: fnb, hlb, hlgB, isdA, isdB, isdG, fhuA, fhuB, dps. Finally, Stauff D. L. et al., (2007). Signaling and DNA-binding activities of the Staphylococcus aureus HssR-HssS two-component system required for heme sensing. J Biol Chem September 7; 282(36):26111-21, describes hrtAB. In order to capture all of the relevant regulatory elements of these genes, we selected 300 base pairs upstream of the start codon of each gene as the promoter region. Each promoter region was then cloned upstream of Green Fluorescent Protein (GFP) to visualize promoter activity in media, blood, and serum. The promoters were cloned in front of GFPmut2 (a GFP variant) such that when the promoter is activated, GFP is transcribed and translated into a fluorescent protein. High fluorescence correlates with high promoter activity.

Materials and Methods

Cloning. For each blood or serum-responsive gene selected from the literature, 300 base pairs of sequence immediately upstream from the start codon was selected as the promoter region. Promoters were amplified from the 502a Staphylococcus aureus genome and cloned in front of GFP using either Gibson assembly (GA) or restriction enzyme (RE) digest. For Gibson assembly, promoters were amplified using primers with homology to the vector backbone. In the table below, primer sequence that matches the promoter is uppercase, while primer sequence that is homologous to the vector backbone is lowercase. For restriction enzyme digest, promoters were amplified using primers with SphI or PstI restriction sites. In the table below, primer sequence containing restriction sites is bold. The vector backbone, plasmid pCN56 (BEI Resources), was amplified using PCR for Gibson assembly, or simply digested with restriction enzymes for restriction enzyme cloning. Note that the dps promoter was never successfully cloned with GFP. After multiple attempts, the dps-GFP cassette was dropped. Final plasmid cassettes for screening are: pCN56-promoter-GFP. Primers used for amplification of promoters are shown in Table 21. Primers used for amplification of vector backbone are shown in Table 22.

TABLE 21
Primers used for amplification of promoters

| | Cloning | Forward Primer | Reverse Primer |
|---|---|---|---|
| Promoter | Method | (BPC#:Sequence) | (BPC#:Sequence) |
| isdA | RE | 366: TATATGCATGCCTATCTGC | 367: GATACCTGCAGGTTGTTT |
| | | GGCATTTGCAG | TCCTCCTAAGGATA |
| | | (SEQ ID NO: 164) | (SEQ ID NO: 165) |
| isdB | RE | 368: GATGCGCATGCCTTCAGT | 369: GATGCCTGCAGGTTGTA |
| | | TGATAACTTTATTA | GAAACAACTCCTAAT |
| | | (SEQ ID NO: 166) | (SEQ ID NO: 167) |
| isdI | RE | 379: GATACGCATGCTTACTCG | 380: GATAGCTGCAGGGGCAA |
| | | TAGCAGTTTTTTGT | TCACTCCTCTATTTT |
| | | (SEQ ID NO: 168) | (SEQ ID NO: 169) |
| isdG | RE | 377: GATGCGCATGCAAACACA | 378: GATGCCTGCAGAATTATC |
| | | AGATAATTGAATTT | CTCTTTTCTGTTTAA |
| | | (SEQ ID NO: 170) | (SEQ ID NO: 171) |
| sbnC | RE | 381: GAATCGCATGCCTTTATT | 382: GAAATCCTGCAGTGTTCA |
| | | AAAGCTGACAAAGTCGTA | GACACCTCGCATTC |
| | | (SEQ ID NO: 172) | (SEQ ID NO: 173) |
| sbnE | GA | 305: taactgactaggcggccgcGAATAC | 306: ccagtgaaaagttcttctcctttactcatTT |
| | | TTCAAGGATTAACATATAGTG | TTTTGTTTCAACTCCCATAAT |
| | | CATTG | TTCATTAATG |
| | | (SEQ ID NO: 174) | (SEQ ID NO: 175) |
| lrgA | GA | 307:taactgactaggcggccgcATGAAA | 308: ccagtgaaaagttcttctcctttactcatT |
| | | AACGATTGAATCCCACTTATTT | GCCTCCTACGTTTGATTTAAC |
| | | TATACG | TAAAG |
| | | (SEQ ID NO: 176) | (SEQ ID NO: 177) |
| lrgB | GA | 309: taactgactaggcggccgcGTTTAGT | 310: ccagtgaaaagttcttctcctttactcatG |
| | | ATTATTATTTGTATTATTATGT | AGCTTGTGCCTCCTCTATTTT |
| | | ACTGGTGCTG | G |
| | | (SEQ ID NO: 178) | (SEQ ID NO: 179) |
| hlgB | GA | 311: taactgactaggcggccgcAAGATC | 312: ccagtgaaaagttcttctcctttactcatA |
| | | CTAGAGATTATTTCGTTCCAG | TCAATTCTGTCCTTTCACCTT |
| | | (SEQ ID NO: 180) | GATTTC |
| | | | (SEQ ID NO: 181) |
| fhuA | GA | 313: taactgactaggcggccgcCTTTCTT | 314: ccagtgaaaagttcttctcctttactcatA |
| | | GCAGATGAATAAATAAATGGT | ATTTCCCTACTTTCAATAAAA |
| | | ATGAGC | TTCTTTCTG |
| | | (SEQ ID NO: 182) | (SEQ ID NO: 183) |
| fhuB | GA | 315: taactgactaggcggccgcTCAAAA | 316: ccagtgaaaagttcttctcctttactcatA |
| | | TGTAACAATGATCAGAGGC | TTGAGCGTCCTCCTTTTTTAA |
| | | (SEQ ID NO: 184) | CTAAATATAAAAAG |
| | | | (SEQ ID NO: 185) |
| ear | GA | 317: taactgactaggcggccgcTGACAC | 318: ccagtgaaaagttcttctcctttactcatTT |
| | | CTGCTAATTCAAACATTATTTG | TAATTGCTCCTTTTTTTATATT |
| | | (SEQ ID NO: 186) | GTAAACGTTTAC |
| | | | (SEQ ID NO: 187) |
| fnb | GA | 319: taactgactaggcggccgcTGTACA | 320: ccagtgaaaagttcttctcctttactcatT |
| | | GGCGATAATTATGAAACACTT | ATAATATCTCCCTTTAAATGC |
| | | AG | AAAATTCATTAATTTTTTTAA |
| | | (SEQ ID NO: 188) | AC |
| | | | (SEQ ID NO: 189) |
| hlb | GA | 321: taactgactaggcggccgcTTCAGG | 322: ccagtgaaaagttcttctcctttactcatA |
| | | CTATCAATAATGCTTTGAAAT | GAAACCTTGTAACAACAGTA |
| | | C | TTTATTGGG |
| | | (SEQ ID NO: 190) | (SEQ ID NO: 191) |
| splF | GA | 323: taactgactaggcggccgcGTTCACC | 324: ccagtgaaaagttcttctcctttactcatTT |
| | | TATATTAAATAGTAAGCGAGA | TTGTGCTCCTCTAAGTATTCG |
| | | AGC | TATAAATATAAGG |
| | | (SEQ ID NO: 192) | (SEQ ID NO: 193) |
| splD | GA | 325: taactgactaggcggccgcATTTTAA | 326: ccagtgaaaagttcttctcctttactcatTT |
| | | ATTTTGATGCATACATTGAAC | TTGTGCTCCTCTGTTTATTCAT |
| | | CCGG | GATGC |
| | | (SEQ ID NO: 194) | (SEQ ID NO: 195) |
| dps | GA | 327: taactgactaggcggccgcATAATA | 328: ccagtgaaaagttcttctcctttactcatA |
| | | GAAATAGAATGTGGAAAACA | TTTAATACACTCCTTAAAATT |
| | | ACATGGC | GTCTACGTC |
| | | (SEQ ID NO: 196) | (SEQ ID NO: 197) |
| SAUSA | GA | 329: taactgactaggcggccgcGATGAT | 330: ccagtgaaaagttcttctcctttactcatT |
| 300_2268 | | GTATGTTTCGAATTTATCAATT | GTTAACACATCCTCGTCGATA |
| | | AACATGTG | ATATTG |
| | | (SEQ ID NO: 198) | (SEQ ID NO: 199) |
| SAUSA | GA | 331: taactgactaggcggccgcCTATCAT | 332: ccagtgaaaagttcttctcctttactcatT |
| 300_2616 | | TATAATGAGATAATGTCATTTT | GGCGTCTCACCTTCCTATC |
| | | TAATTGAGC | (SEQ ID NO: 201) |
| | | (SEQ ID NO: 200) | |
| SAUSA | GA | 333: taactgactaggcggccgcCAGGCC | 334: ccagtgaaaagttcttctcctttactcatG |
| 300_2617 | | TATTTTCTAGGAAATCGATG | ATATATCATTACCTTTCTACA |
| | | (SEQ ID NO: 202) | TTCATTTACATATC |
| | | | (SEQ ID NO: 203) |
| hlgA2 | GA | 201: cgttaactaattaatttaagaaggagatatac | 185: ccagtgaaaagttcttctcctttactcatA |
| | | atACTTCAAATTTTCACAAACT | GAAATCACTTTCTTTCTATTT |
| | | ATTGCG | AATTTTAAGTTCATATATA |
| | | (SEQ ID NO: 204) | (SEQ ID NO: 205) |
| hrtAB | GA | 205: cgttaactaattaatttaagaaggagatatac | 188: ccagtgaaaagttcttctcctttactcatA |
| | | atGTTCATATTGAGTTCATATTT | TCGATTCACTTCTCCCTATTT |
| | | CAACC | CTTC |
| | | (SEQ ID NO: 206) | (SEQ ID NO: 207) |

TABLE 22
Primers used for amplification of vector backbone

| | Cloning | | |
|---|---|---|---|
| Plasmid | Method | Forward Primer | Reverse Primer |
| pCN56 | GA | 197: ATGAGTAAAGGAGAAGAA | 198: ATGTATATCTCCTTCTTAA |
| | (hlgA2, | CTTTTCACTGG | ATTAATTAGTTAACGAATTCG |
| | hrtAB) | (SEQ ID NO: 208) | (SEQ ID NO: 209) |
| pCN56 | GA (all | 197: ATGAGTAAAGGAGAAGAA | 265: gcggccgcctagtcagttaACTCAA |
| | other | CTTTTCACTGG | AGGCGGTAATACGG |
| | promoters) | (SEQ ID NO: 210) | (SEQ ID NO: 211) |

Blood and Serum Samples. For blood samples, 4-8 ml of human blood was drawn into heparinized tubes and frozen. For serum samples, 4-8 ml of human blood was drawn into non-heparinized tubes, rested at room temperature for 15-30 minutes until fully clotted, and centrifuged at 3,000 rpm for 15 minutes. The serum supernatant was carefully removed, transferred to a new tube, and frozen.

Construction of Cell Lines. RN4220 Staphylococcus aureus cells were transformed with pCN56-promoter-GFP plasmids using electroporation. Glycerol stocks of each cell line were preserved as a starting material for the following blood/serum induction assay. Final cell lines for screening are: RN4220+pCN56-promoter-GFP.

Blood and Serum Induction. For each cell line, 1-3 ml tryptic soy broth (TSB) media with 10 μg/ml erythromycin was inoculated with a small scoop of glycerol stock. The culture was grown at 37° C. overnight shaking at 240 rpm. In the morning, the optical density (OD) of the culture was measured and the culture was used to inoculate 1 ml of fresh TSB+erythromycin to an OD of 0.1. This 0.1 OD culture was grown at 37° C. shaking at 240 rpm for 2-3 hours until the OD reached 1-2. The culture was then used to inoculate three separate cultures of 500 μl of freshly thawed blood, serum, or TSB, all with erythromycin, to an OD of 0.1. These three cultures were grown at 37° C. shaking at 240 rpm for 1.5-2 hours. 10 μl of each culture was dropped onto a microscope slide, covered with a coverslip, and viewed with fluorescent microscopy.

Microscopy. Images were taken with an iPhone through the eyepiece of a fluorescent microscope.

Results and Conclusions. The fluorescent images of each Staphylococcus aureus RN4220+pCN56-promoter-GFP cell line cultured in either media (negative control), blood, or serum were read and fluorescence level was scored as summarized in Table 23.

TABLE 23
Relative promoter GFP fluorescence
levels in TSB, Blood or Serum

Fluorescence Level

| Promoter | TSB Media | Blood | Serum |
|---|---|---|---|
| isdA | high | high | high |
| isdB | high | (no sample) | high |
| isdI | low | high | high |
| isdG | very low | high | high |
| sbnC | very low | medium | medium |
| sbnE | very low | low | low |
| lrgA | very low | low | low |
| lrgB | very low | low | none |
| hlgB | very low/none | medium | medium |
| fhuA | high | high | high |
| fhuB | very low | low | low |
| ear | high | high | high |
| fnb | medium | medium | medium |
| hlb | very low/none | medium | medium |
| splF | very low/none | low | low |
| splD | very low/none | very low/none | very low/none |
| SAUSA | low | high | medium |
| 300_2268 | | | |
| SAUSA | very low/none | low | low |
| 300_2616 | | | |
| SAUSA | very low/none | low | low |
| 300_2617 | | | |
| hlgA2 | low | high | medium |
| hrtAB | very low/none | medium | medium |

The promoter for the kill switch requires two essential characteristics. First, the promoter must turn on, or be upregulated, when the cells are exposed to blood or serum. This screen clearly shows a spectrum of promoter activity in the presence of blood or serum; some promoters are very active in blood or serum, and others less so. Depending on the mechanism of activity, different kill genes will likely require promoters with different levels of activity. For example, a kill gene that is extremely lethal, rather than toxic, may require a promoter with very low strength. As various kill genes are tested, it will be possible to return to this list of promoters and rationally build kill switches.

The second requirement is that the candidate promoter must have little or no activity when the cells are not exposed to blood or serum. As the primary purpose of 502a is to colonize the skin before exposure to MRSA, it is critical that the cells grow normally in their intended niche and kill switch activity not interfere with this function. The most desirable kill switch candidate promoters in this screen exhibited very low activity in TSB and medium/high activity in blood or serum including isdG, sbnC, sbnE, hlgB, hlb, SAUSA300_2268, hlgA2, and hrtAB. However, isdI, lrgA, lrgB, fhuB, splF, dps, SAUSA300_2616, SAUSA300_2617 may also be useful promoter candidates for further evaluation. This screen shows several candidate promoters (isdA, isdB, fhuA, ear, and fnb) were active before exposure to blood and serum, so these were deprioritized from the list of potential kill switch promoters.

Additional candidate promoters were selected from the literature for future screening including lukG, lukH, chs, efb, icaB, SAUSA300_1059, SAUSA300_0370, aur, and SAUSA300_0169, as described in Malachowa N, 2011 and Palazzolo-Ballance AM, 2008.

### Example 16. qRT PCR for Genomic Expression of Blood and Serum-Responsive Promoters

In this example, qRT PCR was performed for 20 endogenous Staphylococcus aureus genes found in the literature to be blood and/or serum responsive. The screen was used to help identify candidate blood and/or serum responsive promoters for use in construction of a kill switch molecular modification comprising a cell death gene. Briefly, 502a cells were grown in TSB media, blood, or serum, and RNA was extracted at various time points. In addition, several Staphylococcus aureus genes were tested that are predicted to be unresponsive in blood or serum. These are considered to be candidates for a second promoter to be operably linked to an antitoxin specific for the cell death gene. The results show several genes that are upregulated in blood or serum and a few that are stable in blood or serum.

Growth Procedure. A growth experiment was performed as follows. 4 ml overnight culture of 502a cells was inoculated with a small scoop of competent cells. In the morning, a 125 ml disposable sterile shake flask was inoculated with 50 ml of overnight culture to an optical density (OD) of 0.1. Cells were grown to an OD of 2 (several hours). At OD 2, 500 ul was removed for a T=0 RNA sample. 3×7 ml of the remaining cells were transferred to triplicate 50 ml conical tubes. The tubes were spun, supernatant decanted, washed with PBS, spun again, supernatant removed, and cells resuspended in 7 ml TSB, serum, or blood. Tubes were placed at 37° C. with shaking at 240 rpm. Additional RNA samples were collected at T=1 (tubes were sampled immediately and did not shake at 37° C.), T=15 and T=45 minutes after exposure to serum or blood. RNA sampling method for TSB and serum cultures consisted of 500 ul transferred to a 1.5 ml tube, cells spun at 13,200 rpm for 1 minute, supernatant decanted, and 100 ul of RNALater added. Sampling for blood cultures was the same, except the supernatant was aspirated, and 200 ul of RNALater was added. All samples were stored at −20° C. until further processing (10 months of storage).

qPCR Sample Processing and Data Analysis. RNA extraction and cDNA synthesis was performed. Frozen RNA pellets stored in RNALater were washed once in PBS, extracted using Ambion RiboPure Bacteria kit and eluted in 2×25 ul. RNA samples were DNased using Ambion Turbo DNase kit. Samples with a final concentration less than 50 ng/ul were ethanol precipitated to concentrate DNA. 10 ul of DNased RNA was used in Applied Biosystems High-Capacity cDNA Reverse Transcription kit. qPCR was performed with Applied Biosystems PowerUp SYBR Green Master Mix (10 ul reaction with 1 ul of cDNA). Samples were probed to look for changes in gene expression over time and in different media, and normalized to housekeeping gene, gyrB, using the ΔΔCt method. Ct (cycles to threshold) values for gyrB transcripts were subtracted from Ct values for gene transcripts for each RNA sample. These ΔCt values were then normalized to the initial time point. Primers for qRT PCR screening of candidate serum and/or blood responsive genes are shown in Table 24.

TABLE 24
Primers for qRT PCR screening of candidate serum and/or
blood responsive genes

| | qRT PCR Primers | |
|---|---|---|
| | (BPC#-sequence) | |
| Gene | Forward | Reverse |
| gyrB | BPC802-TTGGTACAGGAATCGGTGGC | BPC803-TCCATCCACATCGGCATCAG |
| | (SEQ ID NO: 212) | (SEQ ID NO: 213) |
| isdA | BPC114-GCAACAGAAGCTACGAACGC | BPC115-AGAGCCATCTTTTTGCACTTGG |
| | (SEQ ID NO: 214) | (SEQ ID NO: 215) |
| isdB | BPC116- | BPC117-TGGCAACTTTTTGTCACCTTCA |
| | GCAACAATTTTATCATTATGCCAGC | (SEQ ID NO: 217) |
| | (SEQ ID NO: 216) | |
| isdI | BPC764-ACCGAGGATACAGACGAAGTT | BPC765-TGCTGTCCATCGTCATCACTT |
| | (SEQ ID NO: 218) | (SEQ ID NO: 219) |
| isdG | BPC120-AACCAATCCGTAAAAGCTTGC | BPC121-AGGCTTTGATGGCATGTTTG |
| | (SEQ ID NO: 220) | (SEQ ID NO: 221) |
| sbnC | BPC768-AGGGAAGGGTGTCTAAGCAAC | BPC769-TCAGTCCTTCTTCAACGCGA |
| | (SEQ ID NO: 222) | (SEQ ID NO: 223) |
| sbnE | BPC124-ATTCGCTTTAGCCGCAATGG | BPC125-GCAACTTGTAGCGCATCGTC |
| | (SEQ ID NO: 224) | (SEQ ID NO: 225) |
| lrgA | BPC126-GATACCGGCTGGTACGAAGAG | BPC127-TGGTGCTGTTAAGTTAGGCGA |
| | (SEQ ID NO: 226) | (SEQ ID NO: 227) |
| lrgB | BPC128-ACAAAGACAGGCACAACTGC | BPC129-GGTGTAGCACCAGCCAAAGA |
| | (SEQ ID NO: 228) | (SEQ ID NO: 229) |
| hlgB | BPC760-TGGTTGGGGACCTTATGGAAG | BPC761-GGCATTTGGTGTTGCGCTAT |
| | (SEQ ID NO: 230) | (SEQ ID NO: 231) |
| fhuA | BPC132-CACGTTGTCTTTGACCACCAC | BPC133-TGGGCAATGGAAGTTACAGGA |
| | (SEQ ID NO: 232) | (SEQ ID NO: 233) |
| fhuB | BPC134-CAATACCTGCTGGAACCCCA | BPC135-GGGTCCGCATATTGCCAAAC |
| | (SEQ ID NO; 234) | (SEQ ID NO: 235) |
| ear | BPC136-CCACTTGTCAGATCTGCTCCT | BPC137- |
| | (SEQ ID NO: 236) | GGTTTGGTTACAGATGGACAAACA |
| | | (SEQ ID NO: 237) |
| fnb | BPC772-CGCAGTGAGCGACCATACA | BPC773-TTGGTCCTTGTGCTTGACCA |
| | (SEQ ID NO: 238) | (SEQ ID NO: 239) |
| hlb | BPC140-CTACGCCACCATCTTCAGCA | BPC141-ACACCTGTACTCGGTCGTTC |
| | (SEQ ID NO: 240) | (SEQ ID NO: 241) |
| splF | BPC142-TGCAATTATTCAGCCTGGTAGC | BPC143-CCTGATGGCTTATTACCGGCAT |
| | (SEQ ID NO: 242) | (SEQ ID NO: 243) |
| splD | BPC144-AGTGACATCTGATGCGGTTG | BPC145-AACACCAATTGCTTCTCGCTT |
| | (SEQ ID NO: 244) | (SEQ ID NO: 245) |
| dps | BPC146-AGCGGTAGGAGGAAACCCTG | BPC147-GTTCTGCAGAGTAACCTTTCGC |
| | (SEQ ID NO: 246) | (SEQ ID NO: 247) |
| srtB | BPC846-TGAGCGAGAACATCGACGTAA | BPC847-CCGACATGGTGCCCGTATAA |
| | (SEQ ID NO: 248) | (SEQ ID NO: 249) |
| emp | BPC854-TCGCGTGAATGTAGCAACAAA | BPC855-ACTTCATGGGCCTTTAGCAACA |
| | (SEQ ID NO: 250) | (SEQ ID NO: 251) |
| sbnA | BPC858-CCTGGAGGCAGCATGAAAGA | BPC859-CATTGCCAACGCAATGCCTA |
| | (SEQ ID NO: 252) | (SEQ ID NO: 253) |
| CH52_360 | BPC834-TTCAACTCGAACGCTGACGA | BPC835-TTGCACCCATTGTTGCACCAT |
| | (SEQ ID NO: 254) | (SEQ ID NO: 255) |
| CH52_305 | BPC838-TTCCTTGGAGCAGTACCACCA | BPC839-CAGCGCAATCGCTGTTAAACTA |
| | (SEQ ID NO: 256) | (SEQ ID NO: 257) |
| CH521670 | BPC842-GCGATTATGGGACCAAACGG | BPC843-ACTTCATAGCTTGGGTGTCCC |
| | (SEQ ID NO: 258) | (SEQ ID NO: 259) |
| clfA | BPC850-TCCAGCACAACAGGAAACGA | BPC851-TAGCTTTCACCAGTTACCGGC |
| | (SEQ ID NO: 260) | (SEQ ID NO: 261) |
| SAUSA300_ | BPC778-GCTTCTACAGCTTTGCCGAT | BPC779-GATTTGGTGCTTACTGCCACC |
| 2268 | (SEQ ID NO: 262) | (SEQ ID NO: 263) |
| SAUSA300_ | BPC774-ACAAGCGCAACAAGCAAGAG | BPC775-TGCGTTTGATACCTTTAACACGG |
| 2616 | (SEQ ID NO: 264) | (SEQ ID NO: 265) |
| SAUSA300_ | BPC152-GGGCTGAAAAAGTTGGCATGA | BPC153-ACGCGTTGTTTTTGACCTCC |
| 2617 | (SEQ ID NO: 266) | (SEQ ID NO: 267) |
| hlgA2 | BPC179-TGATTTCTGCACCTTGACCGA | BPC180-AGCCCCTTTAGCCAATCCAT |
| | (SEQ ID NO: 268) | (SEQ ID NO: 269) |
| hrtAB | BPC713-ACACAACAACAACGTGATGAGC | BPC714-TAACGGTGCTTGCTCTGCTT |
| | (SEQ ID NO: 270) | (SEQ ID NO: 271) |

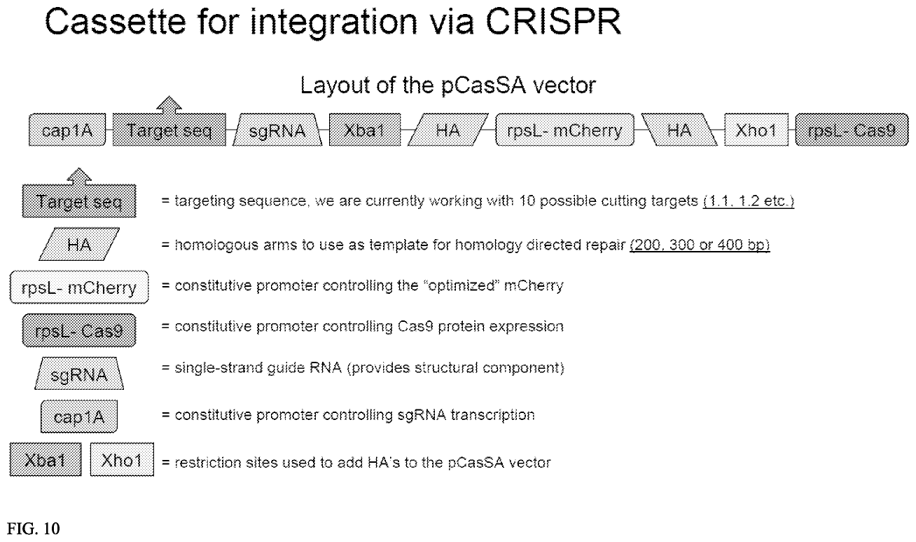

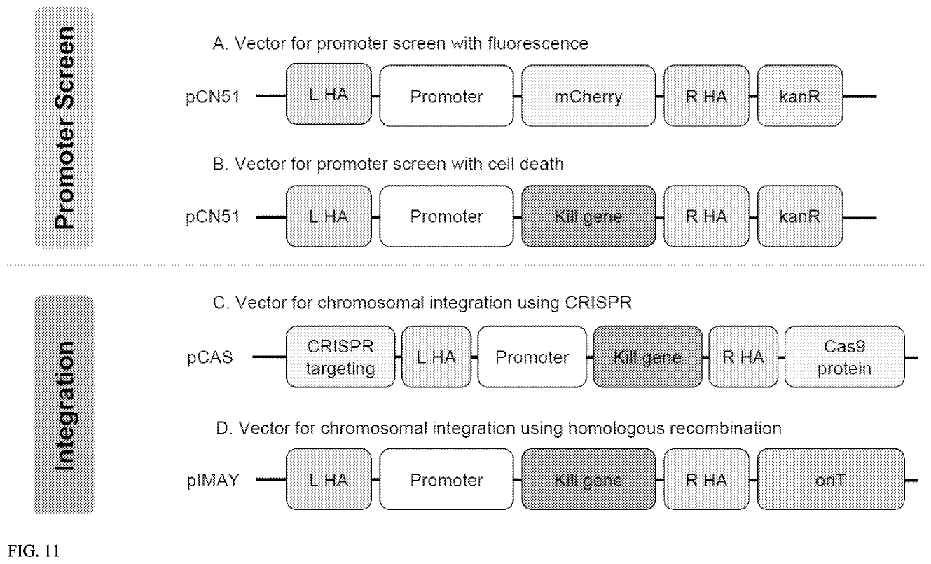

The qPCR results are shown in FIGS. 13A and 13B showing several genes that are upregulated in blood and/or serum. FIG. 13A shows promoter candidates isdA, isdB, hlgA2, hrtAB, isdG, sbnE, lrgA, lrgB, fhuA, fhuB, ear, hlb, splF, splD, dps, and SAUSA300_2617 at 1 min, 15 min and 45 min in serum and fold changes in gene expression vs. media. Preferred serum responsive promoter candidates in this screen include hlgA2, hrtAB, isdA, isdB, isdG, sbnE, ear, and splD, as shown in Table 25 because they exhibit at least a 9-fold increase in gene expression when exposed to serum after 45 min, a slightly delayed response to serum, and are not significantly upregulated at T=1 min.

TABLE 25
Preferred promoter candidates for
serum-responsive genes by qPCR

| Upregulated Gene | Fold Change in Serum at T = 45 min |
|---|---|
| hlgA2 | 9 |
| hrtAB | 209 |
| isdA | 15 |
| isdB | 172 |
| isdG | 42 |
| sbnE | 30 |
| ear | 10 |
| splD | 9 |

FIG. 13B shows candidate promoter activity when exposed to blood of promoter candidates isdA, isdB, hlgA2, hrtAB, isdG, sbnE, lrgA, lrgB, fhuA, fhuB, ear, hlb, splF, splD, dps, and SAUSA300_2617 at 1 min, 15 min and 45 min in serum and fold changes in gene expression vs. media by qPCR. Preferred promoter candidates exhibited a slightly delayed gene expression response at 1 minute, but were significantly upregulated at least 30-fold the 15 and 45 min time points. Preferred promoter candidates for blood-responsive genes by qPCR included isdA, isdB, isdG, sbnE, and SAUSA300_2617, as shown in Table 26.

TABLE 26
Preferred promoter candidates for blood-responsive genes by qPCR

| Upregulated Gene | Fold Change in Blood at T = 45 |
|---|---|
| isdA | 77 |
| isdB | 66 |
| isdG | 69 |
| sbnE | 33 |
| SAUSA300_2617 | 150 |

Another qRT PCR for Genomic Expression of Serum-Responsive Promoters

In this example, qRT PCR is also performed for screening further Staphylococcus aureus genes found in the literature to be blood and/or serum responsive. Briefly, 502a cells were grown in TSB media or serum, and RNA was extracted at various time points. The results show several genes that are highly upregulated in serum. Essentially, the experimental protocol was similar to the example above, except RNA samples were normalized before conversion to cDNA, and samples were collected at T=90 min.

Growth Procedure. The growth experiment was performed as follows. 502a glycerol stock was struck onto a fresh bacterial plate and grown overnight. 3-5 single colonies from the plate were inoculated into a 4 ml culture of BHI media and grown overnight at 37° C. with shaking at 240 rpm. In the morning, the culture was diluted to an optical density (OD) of 0.05 in 5 ml fresh BHI media. Cells were grown at 37° C. with shaking at 150 rpm for several hours to an OD of approximately 1. At this time, samples for RNA were collected for a T=0 time point (1 ml was transferred to a 1.5 ml microcentrifuge tube, centrifuged at 16,000 rpm for 1 minute, supernatant dumped, cells resuspended in 1 ml sterile PBS, centrifuged at 16,000 rpm for 1 minute, supernatant aspirated, cells resuspended in 200 ul RNALater, and stored at −20° C.). The remaining culture was rediluted to an OD of 0.05 in 3 replicate heparinized tubes of 10 ml fresh BHI media or thawed human serum, and incubated at 37° C. with shaking at 150 rpm. Additional samples for RNA were collected at T=90 minutes, and T=180 minutes. For these later samples, one 10 ml tube was centrifuged at 3,000 rpm for 10 minutes, supernatant dumped, cells resuspended in 1 ml PBS, transferred to a 1.5 ml microcentrifuge tube, centrifuged at 16,000 rpm for 1 minute, supernatant aspirated, cells resuspended in 200 ul RNALater, and stored at −20° C.

qPCR Sample Processing and Data Analysis. RNA extraction and cDNA synthesis was performed as follows. Frozen RNA pellets stored in RNALater were washed once in PBS, extracted using Ambion RiboPure Bacteria kit and eluted in 2×50 ul. RNA samples were DNased using Ambion Turbo DNase kit. Samples with a final concentration less than 50 ng/ul were ethanol precipitated to concentrate DNA. 500 ng of DNased RNA was used in Applied Biosystems High-Capacity cDNA Reverse Transcription kit. qPCR was performed with Applied Biosystems PowerUp SYBR Green Master Mix (10 ul reaction with 1 ul of cDNA).

Samples were probed to look for changes in gene expression over time and in different media, and normalized to housekeeping genes, gyrB, sigB, rho, or an average of the three, using the ΔΔCt method. Ct (cycles to threshold) values for housekeeping gene transcripts were subtracted from Ct values for gene transcripts for each RNA sample. These ΔCt values were then normalized to the initial time point. Gene expression at 90 minutes in both TSB and serum were normalized to values at T=0.

Results are shown in FIG. 13C which shows gene expression in serum at T=90 min for promoter candidates hlgB, ear, fnb, splF, splD, clfA, CH52_360, CH52_305, CH52_1670, hlb, lrgB, lrgA, emp, fhuA, fhuB, isdI, isdA, srtB, isdG, sbnE, sbnA, sbnC, and isdB by qPCR compared to TSB. FIG. 13C shows genes upregulated greater than 5-fold in serum include fhuA, fhuB, isdI, isdA, srtB, isdG, sbnE, sbnA, sbnC, and isdB. FIG. 13C shows several genes are upregulated greater than 100-fold after 90 minutes of incubation in serum including isdA, srtB, isdG, sbnE, sbnA, sbnC, and isdB. Specifically, genes in the isd, sbn, and fhu families are upregulated to varying degrees. All of the genes surveyed here have stable expression from T=0 to T=90 minutes in TSB. Several genes from this experiment show high upregulation in serum, while others show stable expression in serum. Both of these characteristics may be useful in construction of a kill switch. For example, a cell death gene may be controlled with a promoter that will upregulate in serum and/or blood, and an antitoxin gene specific for the cell death gene may be controlled with a promoter that will downregulate or remain stable in serum.

### Example 17. Spra1 as a Candidate Cell Death Gene Toxin Using Plasmid Based Induction Systems

In this example, candidate cell death gene sprA1 was evaluated using two different plasmid based induction systems in two Staphylococcus aureus strains. Example 17A. Initial testing of sprA1 as an inhibitor of cell growth of Staph aureus cells (RN4220) was performed using a cadmium inducible promoter. A spra1 toxin gene was cloned behind the cadmium promoter in pCN51 (pTK1). pCN51 vector is a low copy plasmid containing a cadmium inducible promoter.

This version of spra1 contains an antisense which regulates spra1. The full sequence of the sprA1-sprA1AS which is downstream of the cadmium promoter is shown below. This construct is called pTK1.

pTK1: sprA1-sprA1AS: sprA1 toxin gene and ribosome binding site, and antitoxin gene (pTK1 or p001). pTK1 was used in experiments with Cadmium promoter.

(SEQ ID NO: 272)
CGCAGAGAGGAGGTGTATAAGGTGATGCTTATTTTCGTTCACATCATA
GCACCAGTCATCAGTGGCTGTGCCATTGCGTTTTTTTCTTATTGGCTA
AGTAGACGCAATACAAAATAGGTGACATATAGCCGCACCAATAAAAAT
CCCCTCACTACCGCAAATAGTGAGGGGATTGGTGTATAAGTAAATACT
TATTTTCGTTGT

ribosome binding site region
sprA1 toxin gene
sprA1 antitoxin gene CCCCTCACTACCGCAAATAGTGAGGGGATTGGTGTATAAGTAAATACTTAT TTTCGTTGT (SEQ ID NO: 273) sprA1 antitoxin gene

Cadmium is a toxic compound so the first step was to find the sub-inhibitory concentration in which the cadmium has enough of a minimal effect on growth to see a marked delta if sprA1 is having a negative on growth of RN4220. RN4220's were grown overnight in TSB media and diluted down to 0.5 ODs and separated into eight 14 ml culture tubes each containing 3 ml of diluted RN4220 cells. Four concentrations of cadmium were inoculated into 4 tubes with each having no cadmium control. 10 nM, 100 nM, 1 uM and 10 uM were the final cadmium concentrations. The results were evaluated at 2 and 22 hours of growth at 30° C. with 240 RPM shaking (data not shown). After 22 hours the 10 uM Cadmium showed the greatest negative effect. The experiment of determining the minimal sub-inhibitory concentration of cadmium was repeated in duplicate using 10 nM, 100 nM and 1 uM cadmium using Staphylococcus aureus RN4220 cells. After 2 hours, cell growth results from the cadmium test show good tolerance up to 1 uM (data not shown).

Next, 500 nM and 1 uM cadmium was tested using RN4220 cells transformed with pCN54 which has a cadmium inducible promoter was used as an additional control. RN4220 cells were diluted to 0.5 ODs (630 nm) and aliquoted to 4 culture tubes each with 3 ml. Two of the tubes were inoculated with 500 nM and 1 uM cadmium. RN4220 cells containing pCN54 were diluted to 0.5 ODs (630 nm) and aliquoted to 4 culture tubes each with 3 ml. Two of the tubes were inoculated with 500 nM and 1 uM cadmium. All pCN54 growths contained erythromycin 10 as an antibiotic selection. After 2 hours of growth at 30° C., ODs (630 nm) were measured. Results showed good tolerance at 500 nM and 1 uM cadmium. (data not shown). It was concluded that the 4220 cells exhibited good cadmium tolerance at the levels tested except for 10 uM which was too high of a concentration to potentially see a difference between cadmium effects only and an induced toxin.

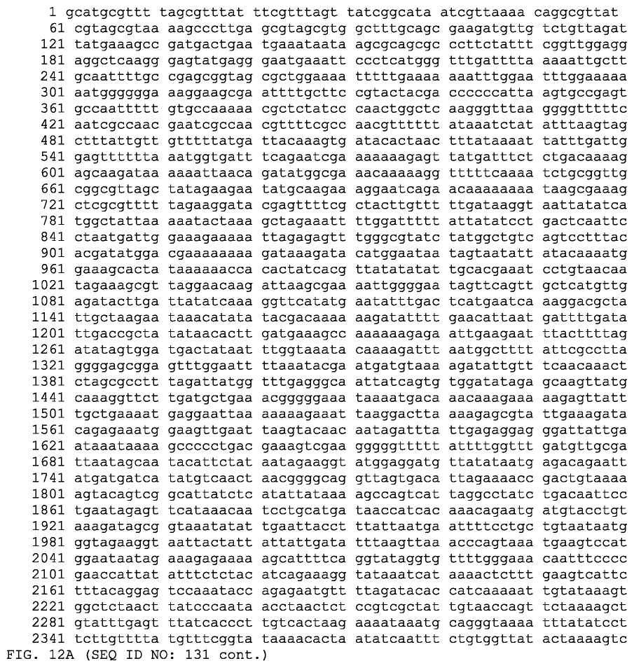

The next experiments included a toxin (sprA1) behind a cadmium promoter on a pCN51 plasmid (pTK1) which had been transformed into RN4220 cells. Both 500 nM and 1 uM concentrations were tested with 2 pTK1 clone picks and RN4220 cells (wt). Overnight cultures of wt RN4220 cells and two clones of pTK1 in RN4220 cells were diluted to 0.5 ODs. Wild-type (WT) RN4220 cells were divided into 3 culture tubes at 3 ml/tube. Two tubes were inoculated with 500 nM and 1 uM cadmium and ODs were read after 2 hours post induction. Each pTK1 clone was divided into 3 culture tubes at 3 ml/tube (6 tubes total). Each pTK1 clone was induced with 500 nm and 1 uM with one being a control. ODs were read after 2 hours post induction. Results are shown in the Table 27 and FIG. 14.

FIG. 14 shows inducible inhibition of cell growth of synthetic microorganism pTK1 cells comprising a cell death toxin gene (sprA1) behind a cadmium promoter on a pCN51 plasmid (pTK1) which had been transformed into Staphylococcus aureus RN4220 cells. OD (630 nm) read at 2 hrs post induction, as shown in Table 27. Wild-type 4220 cells showed good cell growth both in the absence of cadmium and in the presence of 500 nM and 1 uM cadmium. pTK1-1 and pTK1-2 cells showed good growth in the absence of cadmium, but cell growth was significantly inhibited in presence of 500 nM and 1 uM cadmium at 2 hours post induction.

TABLE 27
Staphylococcus aureus RN4220 cells Optical
Density (630 nm) 2 hours post-induction

| Cells | 2 Hr Post OD (630 nm) |
|---|---|
| WT4220 Cad− | 3.0 |
| WT 4220 Cad+ 500 nM | 2.9 |
| WT 4220 Cad+ 1 uM | 2.9 |
| ptK1-1 Cad− | 2.6 |
| pTK1-1 Cad+ 500 nM | 0.19 |
| pTK1-1 Cad+ 1 uM | 0.25 |
| ptK1-2 Cad− | 2.4 |
| pTK1-2 Cad+ 500 nM | 0.16 |
| pTK1-2 Cad+ 1 uM | 0.22 |

The experiment was reproduced and each sample exhibited similar OD (630 nm) results at 2 hrs post-induction (data not shown). In summary, a cadmium tolerance test was performed on wt RN4220 cells and 500 nM-1 uM cadmium showed minimal negative on RN4220 cells. This example shows induction of pTK1 showed suppression of cell growth when induced with cadmium.

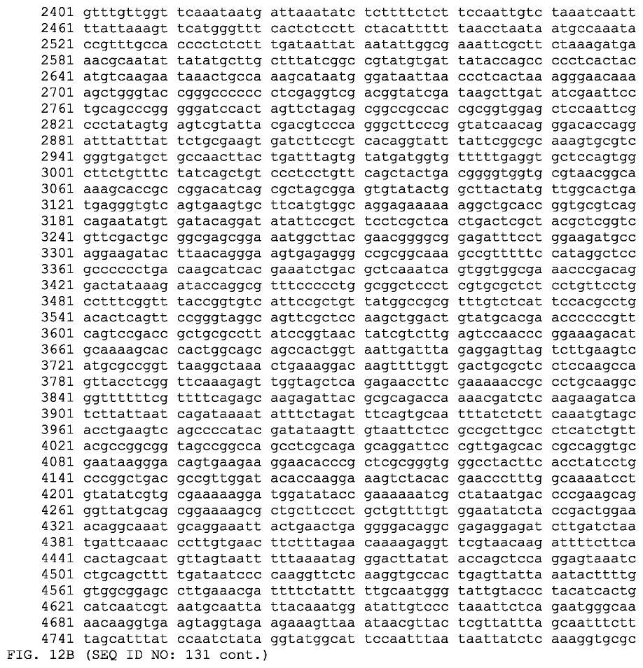

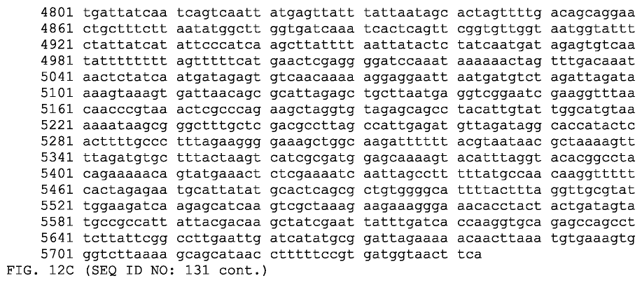

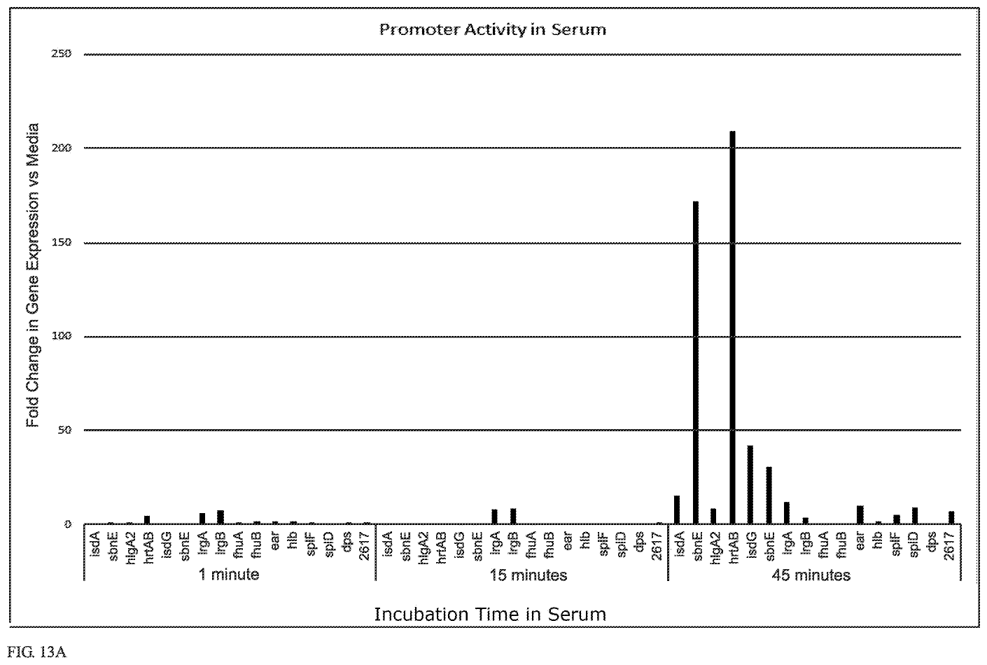

Example 17B. Candidate cell death gene SprA1 was evaluated as an inhibitor of cell growth of Staph aureus cells (502a) using an anhydrotetracycline (ATc) inducible promoter: pRAB11 which is a high copy plasmid containing a tetracycline inducible promoter. Two versions of the sprA1 toxin were cloned behind the tet promoter in pRAB11-2. Clones tested were p174 plasmid containing a deleted spra1 antisense (Das) and p175 plasmid which contains a deleted spra1 antisense plus a missing RBS site. A plasmid map of p174 (pRAB11_Ptet-sprA1) is shown in FIGS. 15A and 15B. FIG. 15A shows a zoomed view of the region of the plasmid containing the Ptet-sprA cassette. FIG. 15B shows the p174 whole plasmid in its native circular form.

Sequences employed in p174 and p175 are shown below. Both p174 and p175 were used in experiments using a tetracycline promoter

p174 sprA1: sprA1 toxin gene and ribosome binding site (p174):

(SEQ ID NO: 274)
CGCAGAGAGGAGGTGTATAAGGTGATGCTTATTTTCGTTCACATCATA
GCACCAGTCATCAGTGGCTGTGCCATTGCGTTTTTTTCTTATTGGCTA
AGTAGACGCAATACAAAATAGGTGACATATAGCCGCACCAATAAAAAT

p175 sprA1(ATG): sprA1 toxin gene beginning at start codon (ribosome binding site removed) (p175):

(SEQ ID NO: 275)
ATGCTTATTTTCGTTCACATCATAGCACCAGTCATCAGTGGCTGTGCC
ATTGCGTTTTTTTCTTATTGGCTAAGTAGACGCAATACAAAATAGGTG
ACATATAGCCGCACCAATAAAAAT

Cell growth. Specifically, tet inducible genes on the pRAB11 vector in 502a cells were grown overnight growths in BHI. The p174 pRAB11-pro-tet-spra1Das exhibited 5.4 OD. The p175 pRAB11-pro-tet-spra1Das(ATG) exhibited 6.2 OD. All 5 overnight cultures were diluted to 0.5 ODs in 1 ml final (14 ml tubes) of BHI-chlor10 (502a wt just BHI). Each cell line was divided into 2 tubes for non-induced and induced anhydrotetracycline (ATc)-10 total.

Induction. Literature shows induction at 100 ng/ml of ATc is effective, so this concentration was selected for induction in these experiments. One tube from each set was induced with 100 ng/ml final concentration. A 1 mg/ml ATc stock in Ethanol was diluted to 100 ug/ml in EtOH. One microliter was added to the appropriate tubes for a final of 100 ng/ml.

The OD's at 630 nm were taken at 2, 4 and 6 hours. The ODs were at 2 and 4 hours were read at a 1/10 dilution while the 6 hour OD was taken at a 1/100 dilution to make sure readings were staying in the linear range.

The 502a's (non-induced and induced) and p174 (pRAB11-pro-tet-spra1Das) tubes were serially diluted to 10e−5 and 10e−6 for dilution plating onto BHI and BHI-chlor10 respectively.

Results are shown in Tables 28 and 29 for ODs, and a plate comparison picture is shown in FIG. 15C.

TABLE 28
Calculations Table for Induction growth curves.

| | O/N | ul O/N | | # of | |
|---|---|---|---|---|---|
| Sample Name | OD | culture | BHI | tubes | conditions |
| 502a wt | 4.7 | 106 | 1 ml | 2 | Un-ind. & |
| | | | | | Induced |
| 502a p174 pRAB11- | 5.4 | 93 | 1 ml | 2 | Un-ind. & |
| ptet-sp a1Das | | | | | Induced |
| 502a p175 pRAB11- | 6.2 | 81 | 1 ml | 2 | Un-ind. & |
| ptet-spa1Das(ATG) | | | | | Induced |

TABLE 29
502a pRAB11 tet induction experiment

| | OD₆₃₀ readings at time point (hours) | | | | | | |
|---|---|---|---|---|---|---|---|
| Sample Name | 0.0 | 1.0 | 2.0 | 3.0 | 4.0 | 5.0 | 6.0 |
| 502a wt | 0.5 | | 4.8 | | 8.0 | | 14 |
| 502a wt + 100 ng ATc | 0.5 | | 4.4 | | 7.1 | | 11 |
| 502a p174 pRAB11-ptet- | 0.5 | | 4.5 | | 7.7 | | 13 |
| spa1Das | | | | | | | |
| 502a p174 pRAB11-ptet- | 0.5 | | 0.7 | | 0.3 | | 0 |
| spa1Das + 100 ng ATc | | | | | | | |
| 502a p175 pRAB11-ptet- | 0.5 | | 4.3 | | 7.7 | | 7 |
| spa1Das(ATG) | | | | | | | |
| 502a p175 pRAB11-ptet- | 0.5 | | 3.8 | | 8.1 | | 13 |
| spa1Das(ATG) + 100 ng | | | | | | | |
| ATc | | | | | | | |

FIG. 15C shows plate dilutions at 10e−5 after 6 hours of induction for uninduced (left) and induced (right) 502a p174 (tet-spra1Das). The Plate on the left=uninduced p174 (tet-spra1Das) at 10e−5 dilution on BHI chlor10. Plate on the right is the induced p174 (tet-spra1Das) at 10e−5 on BHI chlor10. Both plates are samples from post-induction time point of 6 hrs. The plate on the left (Uninduced) was uncountable at 10e−5 but at 10−6 counted ˜720 colonies. The induced plate on the right at 10e−5 produced 16 colonies as shown in Table 30.

TABLE 30
Survival percentage of induced Staphylococcus aureus
502a p174 (tet-spra1Das) cells at 6 hours post-induction

| | | Countable | Calculation for | |
|---|---|---|---|---|
| Condition | Colonies | Dilution | 0.1 mls plated | CFU's/ml |
| Uninduced | 720 | 10e−6 | (720*10e6)/0.1 | 7.2* 10e9 |
| Induced | 16 | 10e−5 | (16*10e5)/0.1 | 1.6* 10e7 |

As shown in Table 30, the survival percentage of induced cells at 6 hours post-induction was calculated as 1.6*10e7/7.2*10e9=0.00222×100=0.222%. The survival percentage of induced Staphylococcus aureus 502a p174 (tet-spra1Das) cells at 6 hours post-induction was only 0.222% compared to uninduced cells. Therefore, the Staphylococcus aureus 502a p174 cells exhibited 100%-0.222%=99.78% measurable average cell death at 6 hours post-induction compared to uninduced cells.

In summary, induction with 100 ng/ml ATc showed good suppression of growth of p174 in 502a cells up to 6 hours post induction of less than 1%, less than 0.5%, or less than 0.25%. Specifically, CFU counts at the end of 6 hours showed a survival percentage of only 0.22% when compared to the uninduced sample and 502a wild type. Induction of p175 control with the deleted RBS site for spra1 showed no negative effects on growth up to 6 hours. In summary, induction of p174 showed suppression of cell growth when induced with ATc. However, induction of p175 control lacking RBS showed no suppression of cell growth when induced with ATc, comparable to 502a wild type cells.

### Example 18. 502a Inducible Plasmid Based Expression of Various Toxin Genes

This example shows the effectiveness of various candidate cell death toxin genes that may be used for a kill switch in Staphylococcus aureus 502a. A plasmid based inducible toxin expression was used for this experiment. pRAB11 is a high copy plasmid in Staph aureus Staphylococcus aureus, and the Ptet promoter is derepressed by the addition of 100 ng/mL of AtC (anhydrotetracycline), allowing for high transcription rates. pRAB11 is described in Helle, Leonie, et al. “Vectors for improved Tet repressor-dependent gradual gene induction or silencing in Staphylococcus aureus.” Microbiology 157.12 (2011): 3314-3323. Four candidate cell death toxin genes were selected for evaluation: sprA1, 187-lysK, Holin, and sprG.

sprA1(PepA1). The gene srpA1 found in Staphylococcus aureus strains has been shown to code for a small membrane toxin PepA1. Sayed, Nour et al “Functional and Structural Insights of A Staphylococcus Aureus Apoptotic-like Membrane Peptide from a Toxin-Antitoxin Module.” Journal of Biological Chemistry, vol. 287, no. 52, 2012, pp 43454-43463, doi:10.1074/jbc.m112.402693. Sayed et al. described how the sprA1 gene codes for the toxin protein called PepA1, which localizes at the bacterial membrane and causes cell death. This is part of a type I toxin antitoxin system in Staphylococcus aureus, and has been evolutionarily preserved in their genome.

187-lysK. This is an engineered phage lysin protein from the Staphylococcus aureus phage K. Horgan, Marianne, et al. “Phage lysin LysK can be truncated to its CHAP domain and retain lytic activity against live antibiotic-resistant staphylococci.” Applied and environmental microbiology 75.3 (2009): 872-874. O'Flaherty et al. designed and truncated this peptide and determined it to still retain is lytic activity for many Staphylococcus aureus strains. O'Flaherty, S., et al. “The recombinant phage lysin LysK has a broad spectrum of lytic activity against clinically relevant staphylococci, including methicillin-resistant Staphylococcus aureus.” Journal of bacteriology 187.20 (2005): 7161-7164.

holin. The holin toxin we tested in this experiment is part of the genome of many lytic phages that target Staphylococcus aureus. It has been shown to disrupt cell growth in E. coli when induced from a plasmid expression vector by forming lesions in the cellular membrane. Song, Jun, et al. Journal of General Virology 97.5 (2016): 1272-1281.

sprG. The coding region termed sprG is part of another type I toxin antitoxin system in Staphylococcus aureus. Two peptides are coded for in the same reading frame of sprG, and both have been shown to cause cell death when induced. Pinel-Marie et al. Cell reports 7.2 (2014): 424-435.

Materials. Various synthetic strains were prepared as shown below and 502a wt was also employed. Strains include:
BP_068 (502a pRAB11-Ptet-sprA1)
BP_069 (502a pRAB11-Ptet-187lysK)
BP_070 (502a pRAB11-Ptet-holin)
BP_071 (502a pRAB11-Ptet-sprG1)
BP_001 (502a wt).

Growth Media used in this example included BHI broth media (37 g/L) (Alpha Biosciences), BHI agar plates, BHI Chloramphenicol (10 μg/mL (Teknova)) agar plates, and BHI Chlor (10 μg/mL (Teknova))+AtC (100 ng/mL (Alfa Aesar)) agar plates. Table 31 below shows a list of oligonucleotide sequences used for constructing the plasmids.

TABLE 31
List of oligos and their sequences used for constructing
plasmids

| Oligo Name | DNA sequence (5′-3′) |
|---|---|
| BPC_670 | GCTCAGATCTGTTAACGGTACCATCATACTC (SEQ ID |
| | NO: 276) |
| BPC_671 | CACTGGCCGTCGTTTTACAAC (SEQ ID NO: 277) |
| BPC_672 | gagtatgatggtaccgttaacagatctgagcCGCAGAGAGGAGGTGTATA |
| | AGGTG (SEQ ID NO: 278) |
| BPC_674 | gagtatgatggtaccgttaacagatctgagcATGGTGGCATTACTGAAATC |
| | TTTAGAAAG (SEQ ID NO: 279) |
| BPC_675 | gagtatgatggtaccgttaacagatctgagcATGGCACTGCCTAAAACGG |
| | G (SEQ ID NO: 280) |
| BPC_676 | gagtatgatggtaccgttaacagatctgagcATGGCTAATGAAACTAAAC |
| | AACCTAAAGTT (SEQ ID NO: 281) |
| BPC_677 | gttgtaaaacgacggccagtgCCCGGGCTCAGCTATTATCA (SEQ ID |
| | NO: 282) |
| BPC_678 | gttgtaaaacgacggccagtgGCGGCCGCCCATGCATGC (SEQ ID |
| | NO: 283) |

Table 32 shows the DNA sequence and amino acid sequence for toxin genes. sprA1, 187-lysK, holin, and sprG were tested in this experiment. The toxin gene sprG has two reading frames which have both been shown to have toxin activity in Staphylococcus aureus. The shorter sequence is in bold.

TABLE 32
DNA and amino acid sequences for toxins

| Toxin | DNA Sequence | Protein Sequence |
|---|---|---|
| sprA1 | ATGCTTATTTTCGTTCACATCATAGC | LIFVHIIAPVISGCAIAFFSY |
| | ACCAGTCATCAGTGGCTGTGCCATT | WLSRRNTK |
| | GCGTTTTTTTCTTATTGGCTAAGTAG | (SEQIDNO:285) |
| | ACGCAATACAAAATAG | |
| | (SEQ ID NO: 284) | |
| 187- | Atggcactgcctaaaacgggtaaaccaacggcaaaaca | MALPKTGKPTAKQVVDW |
| lysK | ggtggttgactgggcaatcaatttaatcggcagtggtgtcg | AINLIGSGVDVDGYYGRQ |
| | atgttgatggttattatggtcggcaatgttgggatttacctaac | CWDLPNYIENRWNEKTP |
| | tatatttttaatagatactggaactttaagacaccaggcaacg | GNARDMAWYRYPEGFKV |
| | caagagatatggcatggtatagatatcctgaagggtttaaa | FRNTSDFVPKPGDIAVWT |
| | gtgtttagaaacacttctgattttgtccctaaaccaggtgatat | GGNYNWNTWGHTGIVVG |
| | agcagtgtggacaggtggtaattacaattggaacacttggg | PSTKSYFYSVDQNWNNSN |
| | gacacactggtattgttgtaggtccatcaactaaaagttactt | SYVGSPAAKIKHSYFGVT |
| | ttatagtgtagatcagaattggaataactctaactcttacgttg | HFVRPAYKAEPKPTPPLDS |
| | gtagtcctgcagcaaagataaaacatagttattttggtgtaa | TPATRPVTGSWKKNQYGT |
| | ctcattttgttagacccgcatacaaagcagaaccgaaacct | WYKPENATFVNGNQPIVT |
| | acaccaccactggacagtacaccggcaactagaccagtta | RIGSPFLNAPVGGNLPAGA |
| | caggttcttggaaaaagaaccagtacggaacttggtataaa | TIVYDEVCIQAGHIWIGYN |
| | ccggaaaatgcaacatttgtcaatggtaaccaacctatagta | AYNGNRVYCPVRTCQGV |
| | actagaataggttctccattcttaaatgctccagtaggcggt | PPNQIPGVAWGVFK |
| | aacttaccggcaggggctacaattgtatatgacgaagtttgt | (SEQ ID NO: 287) |
| | atccaagcaggtcacatttggataggttataatgcttacaac | |
| | ggtaacagagtatattgccctgttagaacttgtcaaggtgttc | |
| | cacctaatcaaatacctggcgttgcctggggagtattcaaa | |
| | (SEQ ID NO: 286) | |
| Holin | Atggctaatgaaactaaacaacctaaagttgttggaggaat | MANETKQPKVVGGINFST |
| | aaactttagcacaagaactaagagtaaaacattttgggtag | RTKSKTFWVAIISAVAVFA |
| | caattatatcagcagtagcagtatttgctaatcaaattacagg | NQITGAFGLDYSAQIEQGV |
| | tgcttttggtttagactactcagctcaaattgagcaaggtgta | NIIGSILTLLAGLGIIVDNN |
| | aatatcataggttctatactaacattattagcaggtttaggtatt | TKGLKDSDIVQTDYIKPRD |
| | attgttgataataatactaaaggtcttaaagatagtgatattgt | SKDPNEFVQWQANANTA |
| | tcaaacagattatataaaacctcgtgatagtaaagaccctaa | STFELDNYENNAEPDTDD |
| | tgaatttgttcaatggcaagcaaatgcaaacacagctagca | SDEVPAIEDEIDGGSAPSQ |
| | ctttcgaattagacaactatgaaaacaatgcagaacctgata | DEEDTEEHGKVFAEEEVK |
| | cagatgatagtgatgaagtacctgctattgaagatgaaattg | (SEQ ID NO: 289) |
| | atggcggttcagcaccttctcaagatgaagaagataccga | |
| | ggaacacggtaaagtatttgcagaggaggaagttaagtag | |
| | (SEQ ID NO: 288) | |
| sprG | ATGGTGGCATTACTGAAATCTT | TAGMVALLKSLERRRLMITIST |
| | AAAGGAGACGCCTAATGATTACAA | MLQFGLFLIALIGLVIKLI |
| | TTAGTACCATGTTGCAGTTTGGTT | ELSNKK |
| | TATTCCTTATTGCATTGATAGGTC | (SEQ ID NO: 291) |
| | TAGTAATCAAGCTTATTGAATTAA | |
| | GCAATAAAAAATAA | |
| | (SEQ ID NO: 290) | |
| sprA2 | ATGTTCAATTTATTAATTAACATCAT | MFNLLINIMTSALSGCLVA |
| | GACTTCAGCTTTAAGCGGCTGTCTT | FFAHWLRTRNNKKGDK |
| | GTTGCGTTTTTTGCACATTGGTTACG | (SEQ ID NO: 305) |
| | AACGCGCAACAATAAAAAAGGTGA | |
| | CAAATAA | |
| | (SEQ ID NO: 304) | |

Methods

Plasmid Construction was performed as follows.
1) PCR amplify pRAB11 backbone using primers BPC_670 and BPC_671 using an empty vector as a template.
2) PCR amplify toxin genes from synthesized plasmid DNA (Genscript). This allows for designing a primer that binds to the plasmid backbone downstream of the target gene, negating the need to design and order unique primers for both ends of each gene.
3) Primer pairs
   a) sprA—BPC_672/BPC_677
   b) 187-lysK—BPC_675/BPC_678
   c) Holin—BPC_676/BPC_678
   d) sprG—BPC_674/BPC_678
4) Run PCR products to check for correct size, digest the template DNA with DpnI (NEB), and clean up the reactions with a Zymo spin column.
5) Assemble the cleaned up PCR products by Gibson Assembly and transform into electrocompetent IM08B E. coli cells using the manufacturers protocol (NEB).
6) Verify correct sequences for the promoter and toxins on the plasmids.
7) Transform sequence verified plasmids into electrocompetent Staphylococcus aureus.

Growth Experiments were performed as follows.
1) Start overnight cultures of each strain in 5 mL BHI broth media. Add 10 ug/mL Chloramphenicol to the media for strains BP_068-BP_071.
2) Perform a 1:100 dilution of the overnight culture into fresh BHI. Add 10 ug/mL chloramphenicol to the media for strains BP_068-BP_071. Incubate at 37° C. shaking at 250 rpm for 2 hours. Streak a plate of each strain and incubate overnight at 37° C. to confirm cultures are good.
3) Take OD600 readings of 2 hr cultures and dilute the cultures to an OD of 0.05
   a) Each strain gets (4) 5 mL tubes with BHI broth
   b) The following table shows the recorded OD readings, and the calculated amounts of each culture used to inoculate fresh cultures to an OD of 0.05.
      Table 33

TABLE 33
Starting OD₆₀₀ readings.

| | | | Calculated |
|---|---|---|---|
| Strain | OD600 | uL inoculum | starting OD |
| BP_068 | 2.1 | 119 | 0.0499 |
| BP_069 | 1.7 | 147 | 0.0498 |
| BP_070 | 2.0 | 125 | 0.05 |
| BP_071 | 1.8 | 139 | 0.05 |
| BP_001 | 1.7 | 147 | 0.0498 |

4) Save 100 uL sample of each culture for dilution plating. (3 plates/culture)
5) Incubate cultures at 37° C. until the OD reaches 0.5. Add 150 ng/mL anhydrotetracycline (AtC) to 2 tubes for each strain and label them with a + to indicate they received the inducer (derepressor). Continue to grow the cells for another 4 hours taking samples as described below.
6) Take OD600 readings at T=30 min, 60 min, 120 min, and 240 min. Record values in the table below
   a) Perform dilution plating at T=0, 60 min, and 240 min, and plate the correct dilution on the following plates (BHI, BHI Chlor10, BHI Chlor10+AtC 0.1)

Cfu investigation was performed as follows.
1) Identify BHI (Chlor 10, AtC 0.1) agar plates with colonies growing on them from strains containing plasmids with toxin genes present. Plates from T240 would be best.
2) If possible, pick 8 colonies per strain. Patch colonies to new BHI (Chlor 10, AtC 1) agar plate, and perform Staphylococcus aureus lysis procedure. Use 5 uL of the lysis reaction as the template for colony PCR using primer DR_215/DR_216 using a HF polymerase, such as Q5/Phusion.
   a) Reactions that produce a good band, perform DpnI digest for 1 hr, and column purify PCR reaction. Send purified product for sequencing using primers DR_215/DR_216.

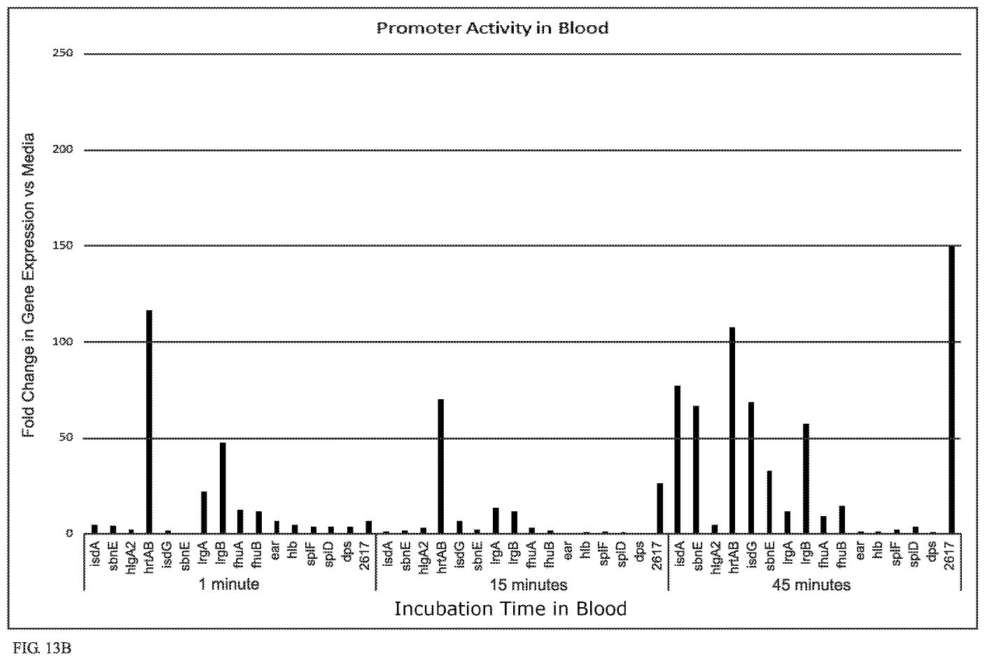

Calculated OD600 readings were taken at T=0, 30, 60, 120, and 240 min after induction. All values after TO are the average of 2 tubes. Results are shown in Table 34 and FIG. 16. The + indicates the cultures that received AtC, and the − indicates the cultures that did not receive any additional factors. FIG. 16 shows calculated OD600 values vs. time. The dashed lines represent the cultures that received 150 ng/mL AtC at T=0. FIG. 16 shows the sprA gene that codes for the PepA1 toxin protein showed the largest reduction in viable 502a Staphylococcus aureus cells after 4 hours of growth post induction.

Specifically, FIG. 16 shows cell growth pre- and post-induction of four synthetic strains derived from Staphylococcus aureus 502a having a plasmid based inducible expression system comprising four different cell death gene candidates sprA1, 187-lysK, Holin, and sprG. The candidate cell death genes had been cloned behind an tetracycline inducible promoter on pRAB11 plasmids and transformed into Staphylococcus aureus 502a cells. Calculated OD600 readings were taken at T=0, 30, 60, 120, and 240 min after induction of AtC induced (+) strains illustrated by dashed lines (- - - - - -) and uninduced (−) strains indicated by solid lines (———) for BP_068 (502a pRAB11-Ptet-sprA1), BP_069 (502a pRAB11-Ptet-187lysK), BP_070 (502a pRAB11-Ptet-holin), and BP_071 (502a pRAB11-Ptet-sprG1) and compared to BP_001 (502a wt) in BHI media. Each of the induced (+) strains BP_068 (sprA1), BP_069 (187lysK) and BP_070 (holin) exhibited both (i) good cell growth pre-induction and (ii) significant inhibition of cell growth post-induction. BP_068 (+) exhibited the best inhibition of cell growth at each time point T=30, T=60, T=60, T=120 and T=240 min post-induction, so the sprA1 gene was selected for initial further development of a kill switch in Staphylococcus aureus 502a.

TABLE 34
Calculated OD600 at T = 0, 30, 60, 120,
and 240 min after induction as shown in FIG. 16

| | Average OD600 Readings | | | | |
|---|---|---|---|---|---|
| Strain +/− ind. | T0 | T30 min | T60 min | T120 min | T240 min |
| 68+ | 1.05 | 0.05 | 0 | 0 | 0 |
| 68− | 1.05 | 1.45 | 2.05 | 3.4 | 5.7 |
| 69+ | 1 | 0.15 | 0 | 0.1 | 0 |
| 69− | 0.95 | 1.25 | 1.75 | 2.8 | 5.5 |
| 70+ | 1 | 0.8 | 0.7 | 0.5 | 0.4 |
| 70− | 0.9 | 1.3 | 1.8 | 2.9 | 5.6 |
| 71+ | 1 | 1.1 | 1.4 | 2.1 | 5.2 |
| 71− | 1 | 1.5 | 2 | 3.1 | 5.8 |
| 502a+ | 1.1 | 1.15 | 1.45 | 2.1 | 3.7 |
| 502− | 1.15 | 1.45 | 2.15 | 3.5 | 5.4 |

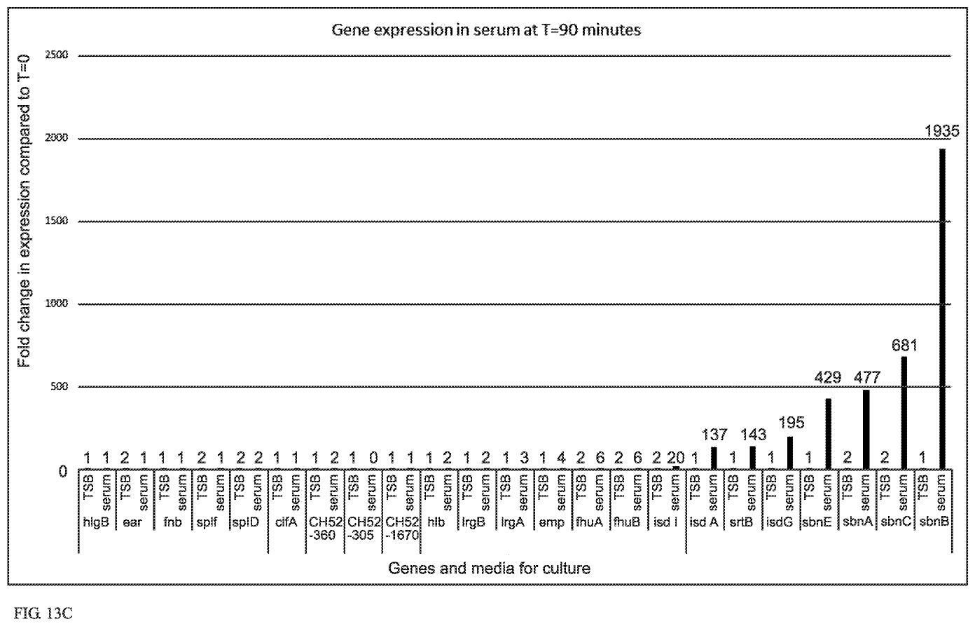

Table 35 below and FIG. 17 show colony forming units calculated from plate counts of diluted liquid culture samples. FIG. 17 shows a bar graph showing difference in the colony forming units/mL between T=0 (gray) and 240 min(black).

TABLE 35
CFUs calculated from plate counts
of diluted liquid culture samples.

| | AtC | T0 (cfu/mL) | T240 (cfu/mL) |
|---|---|---|---|
| 68 | 68+ | 2.85E+09 | 7.50E+01 |
| | 68− | 3.12E+09 | 7.75E+09 |
| 69 | 69+ | 8.75E+09 | 6.30E+03 |
| | 69− | 1.40E+09 | 1.10E+09 |
| 70 | 70+ | 5.25E+09 | 4.75E+04 |
| | 70− | 6.05E+09 | 1.41E+11 |
| 71 | 71+ | 3.00E+09 | 1.04E+11 |
| | 71− | 1.34E+09 | 2.69E+11 |
| 502 | 502a+ | 1.29E+09 | 2.07E+11 |
| | 502a− | 1.45E+09 | 2.62E+11 |

This example investigated the effectiveness of multiple toxin genes when operably linked to an inducible promoter at disrupting cell viability when grown in complex rich media. Two native Staph toxins sprA and sprG, one chimeric phage toxin we have termed 187lysK, and one more phage holin toxin were tested using a plasmid based inducible expression system. The sprA1 gene that codes for the PepA1 toxin protein showed the largest reduction in viable 502a Staphylococcus aureus cells after 4 hours of growth post induction. The sprA1 gene was selected for initial further development of a kill switch in Staphylococcus aureus 502a.

### Example 19. Induced Expression of GFP from the Genome in Strain BP_076 (502a ΔsprA1::Pₜₑₜ-gfp)

Overview. In this example the expression of green fluorescent protein (GFP) from the genome of a Staphylococcus aureus 502a variant strain (BP_076) was confirmed with quantitative polymerase chain reaction (qPCR). The gfp gene was integrated into the genome along with a tetracycline-inducible promoter (Pₜₑₜ) and tetracycline repressor protein gene (tetR). The Pₜₑₜ-gfp expression system was introduced into the genome via the suicide plasmid pIMAYz to allow for controllable expression of a recombinant gene. The wild-type strain (BP_001) served as the negative control and a strain carrying a high-copy plasmid with the same Pₜₑₜ-gfp expression system served as the positive control. Due to its lower toxicity than tetracycline, anhydrotetracycline (aTc) was used to induce expression at 100 ng/mL.

Summarized Results. When comparing the t=0 min samples of BP_055 and BP_076 to BP_001, the qPCR data shows minor GFP expression before induction (indicating that Pₜₑₜ is leaky); however, the expression fold change after induction is still clearly evident. Different expression patterns are seen between plasmid-based and integrated gfp. Integrated gfp shows a sustained increase in expression throughout the assay, whereas plasmid-based gfp shows a high upregulation at 30 minutes and nearly no expression at 90 minutes. The difference in expression between BP_076 and BP_055 is due to the copy number of tetR per cell in each strain. BP_076 has one copy per cell, whereas BP_055 has 300-500 copies depending on the number of plasmids in each cell. The high amount of total TetR protein present in the BP_055 culture clearly exceeded the amount of aTc used for induction by the end of the assay, which lead to repression of gfp expression.

Bacteria Strains and Materials.

Strains

BP_001 (Staphylococcus aureus 502a)

BP_055 (SA 502a, p229_pRAB11-Ptet-GFP)

BP_076 (SA 502a, ΔsprA1::Ptet-GFP)

Brain Heart Infusion (BHI) media, BHI+Chloramphenicol (10 μg/mL) agar plates, Anhydrotetracycline (aTc) were employed.

Samples were RNA (1 mL culture): t=0, 30 and 90 minutes.

Methods—Strain Construction
1. In order to make a modification in the genome of Staph aureus Staphylococcus aureus, we must first add the required genetic elements to a plasmid capable of making those modifications.
2. The plasmid backbone is an E. coli-Staphylococcus aureus shuttle vector called pIMAYz, and has chloramphenicol resistance, a low copy E. coli origin of replication, a low copy temperature sensitive Staphylococcus aureus origin of replication (permissible replication at 30° C., but not at 37° C.), the secY toxin under the control of a Ptet promoter, and a lacZ gene for blue/white screening during integration into Staphylococcus aureus.
3. The plasmid was constructed using linear PCR products that were assembled into a circular construct using Gibson Assembly
   a. Use primers DR_022/DR_023 to PCR amplify the backbone of the pIMAYz vector to linearize it for use in downstream assemblies. The background template DNA must be enzymatically digested with DpnI (NEB) per manufacturer's instructions prior to further use.
   b. Use primers DR_255/DR_241 to PCR amplify the tetR-Ptet-GFP region using the pRAB11 plasmid as the template.
   c. Use primers DR_256/DR_257, and DR_240/DR_236 to PCR amplify 1 kb regions from the Staphylococcus aureus 502a genome. These will be used as homology arms to target the region for integration into the Staphylococcus aureus genome.
   d. These linear fragments are then assembled into a circular plasmid with the Gibson Assembly Master mix (NEB) per manufacturer's instructions and transformed into IM08B cells.
4. Once the sequence of the new plasmid DNA can be confirmed, 50 mL cultures are started to obtain a sufficient amount for transformation into Staphylococcus aureus 502a by electroporation.
5. Integration into Staphylococcus aureus by homologous recombination
   a. Use between 1 and 5 micrograms of plasmid DNA to electroporate into Staphylococcus aureus. Recover at 37° C. for 1 hour, and plate on BHI+10 ug/mL chloramphenicol and 100 ug/mL x-Gal, and incubate overnight at 37° C.
   b. The following day pick multiple blue colonies and start 5 mL BHI broth cultures at room temp, and allow them to grow in a rotary shaking unit for 12-20 hours.
   c. Perform and plate serial dilutions (usually 10⁻⁴-10⁻⁶) on BHI+1 ug/mL anhydrotetracycline (AtC) and 100 ug/mL X-gal. Incubate overnight at 37° C.
   d. The following day, pick and screen white colonies by patching onto BHI, BHI+1 ug/mL anhydrotetracycline (AtC) and 100 ug/mL X-gal, and BHI+10 ug/mL chloramphenicol and 100 ug/mL x-Gal agar plates to confirm chlor sensitivity and AtC resistance.
   e. Colonies showing the desired phenotypes should be screened by PCR with primers DR_237/DR_238. Colonies that have taken the new genes should produce a 4.4 kb band, and colonies that have reverted back to wild type should have a 2.86 kb band. Several positive clones should be sequenced to verify the correct sequences, and one of the sequence verified clones to be picked for use in downstream experiments.

Cell Growth Procedure
1. Start overnight cultures of each strain in BHI broth media (5 mL) and incubate with agitation (37° C., 240 rpm). Add chloramphenicol (final concentration 10 g/mL) to the media for BP_055.
2. Measure optical density (OD) of overnight culture and record.
   The optical density (OD) of the cultures was measured at 630 nm, fresh media served as the blank The OD of the overnight cultures is denoted as the initial OD. The inoculum transferred to 5 mL of fresh media reduced the OD to 0.05 so that the new cultures would be in the exponential growth phase two hours after inoculation, as shown in Table 36.

TABLE 36
OD of cultures for P_001, BP_055 and BP_076

| Strain | Initial OD | Inoculum for 5 mL [μL] | OD at 2 hr |
|---|---|---|---|
| BP_001 | 8.2 | 30.5 | 1.01 |
| BP_055 | 9.1 | 27.5 | 0.88 |
| BP_076 | 8.7 | 28.7 | 1.01 |

3. Dilute overnight cultures to 0.05 OD in fresh BHI (5 mL) in 2×14 mL culture tubes per culture; again add chloramphenicol to the BP_055 cultures.
4. Incubate with agitation (37° C., 240 rpm) until OD reaches 0.5-1 (“2 hr culture”).
5. Remove 1 mL of culture for t=0 min RNA samples and transfer them to 1.5 mL microtubes. Spin down the samples (16,000×g, 1 min, RT), aspirate off supernatant and resuspend the pellet in 200 μL RNAlater. Allow them to incubate for a few minutes at room temperature (RT) and then store at −20° C.
6. Add aTc (4 μL, 100 μg/mL) to first 14 mL culture tube for each strain. Add 4 μL 100% ethanol to second tube for each strain as induction controls (the aTc was solvated in 100% ethanol).
7. Incubate the cultures with agitation (37° C., 240 rpm) until other sampling timepoints.
8. Repeat RNA sampling at t=30 and 90 mins, measure OD at t=90 mins. qPCR Sample Processing and Data Analysis
   RNA was extracted from frozen cell pellets stored in RNALater using Ambion RiboPure Bacteria Kit per protocols in example above. The gfp expression level was normalized to the housekeeping gene gyrB and quantitated using the ΔΔCt method, see the primer sequences in Table 37.

TABLE 37
Sequences of qPCR primers.

| Target | Database Number | Sequence |
|---|---|---|
| gyrB | BP 802 | 5′-TTGGTACAGGAATCGGTGGC |
| | | (SEQ ID NO: 212) |
| gyrB | BP 803 | 5′-TCCATCCACATCGGCATCAG |
| | | (SEQ ID NO: 213) |
| gfP | BP 195 | 5′-CTGTCCACACAATCTGCCCT |
| | | (SEQ ID NO: 292) |
| gfP | BP 196 | 5′-TGCCATGTGTAATCCCAGCA |
| | | (SEQ ID NO: 293) |

Primer sequences used for plasmid and strain construction are shown in Table 38.

TABLE 38
Primers used for plasmid and strain construction

| Primer | |
|---|---|
| Name | ssDNA sequence (5′-3′) |
| DR_022 | Caagcttatcgataccgtcgacctc |
| | (SEQ ID NO: 294) |
| DR_023 | Gggatccactagttctagagcgg |
| | (SEQ ID NO: 295) |
| DR_237 | GCAACTGGTACATCACAATTGGTACTCTCAC |
| | (SEQ ID NO: 296) |
| DR_238 | GACCACGCATACCTATCTATAAACGGACAATG |
| | (SEQ ID NO: 297) |
| DR_255 | GTCCAATTAGATGGCATGTAACTGGGCAGTGTCTTAAAAAAT |
| | CG (SEQ ID NO: 298) |
| DR_241 | CAGGCCAATTTGGCATAGAGCCGGATGTGCTGCAAGGCGATT |
| | AAGTTGGGTAACG (SEQ ID NO: 299) |
| DR_256 | GTTACATGCCATCTAATTGGACAAATTCTATGAGAGTAGATT |
| | TTG (SEQ ID NO: 300) |
| DR_257 | GCCAAATCGCTTTCGTGTATACGATTCCCAGTC |
| | (SEQ ID NO: 301) |
| DR_240 | GGCTCTATGCCAAATTGGCCTGATGAGTTC |
| | (SEQ ID NO: 302) |
| DR_236 | gctctagaactagtggatcccGGCGATTTTATTGTGACAAGA |
| | GACTGAAGAGC (SEQ ID NO: 303) |

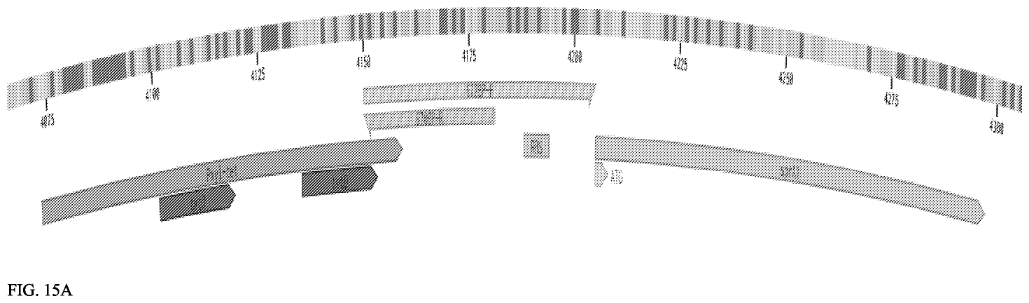

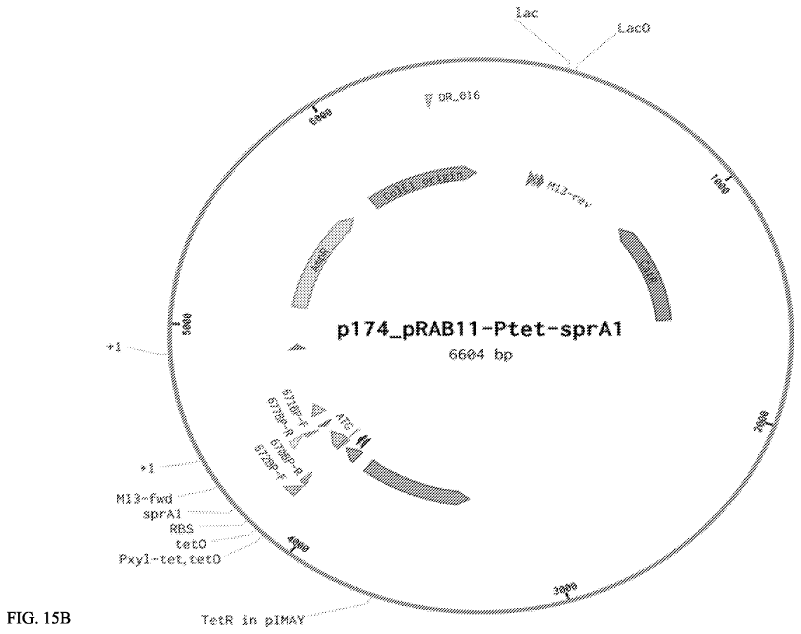

FIG. 19 shows a map of the genome for Strain BP_076 (SA 502a, ΔsprA1::Ptet-GFP).
FIG. 20 shows a map of plasmid constructed for making genomic integration in Staphylococcus aureus.

Results. The t=0 samples of both strains carrying the Ptet-gfp system showed some GFP expression before induction, Table 2 shows the Ct values of the three investigated strains at t=0. The wild-type strain BP_001 amplification curve crossed the threshold (0.4) after 30 cycles, which may be attributed to some form of unspecific amplification or primer dimer formation.

Table 39 shows the Cycles to Threshold (Ct) values prior to expression induction for the wild-type strain BP_001, plasmid based Pₜₑₜ-gfp BP_055 carry strain and Pₜₑₜ-gfp genetically modified strain BP_076 are shown. The threshold was set to 0.4.

TABLE 39
Cycles to Threshold (Ct) values prior to induction
for BP_001, BP_055 and BP_076

| Strain | BP_001 | BP_055 | BP_076 |
|---|---|---|---|
| Ct Value | 33.65 ± 0.61 | 22.99 ± 0.06 | 23.09 ± 0.10 |

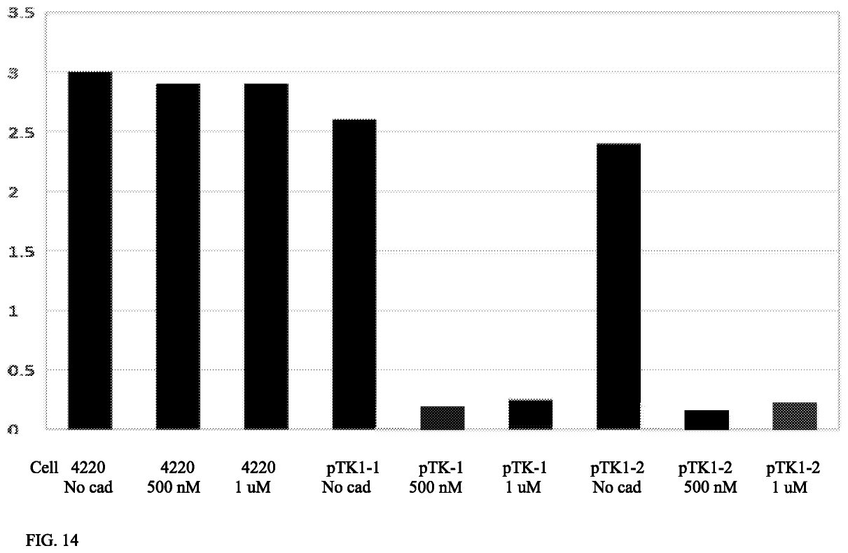

The basal expression level of GFP was accounted for in the ΔΔCt calculations by normalizing the experimental timepoints (t=30 min, 90 min) to the control timepoint (t=0) for each strain individually. The expression levels of GFP determined by qPCR are displayed below in FIG. 18. FIG. 18 shows GFP expression fold change of induced (+) and uninduced (−) subcultures of Staphylococcus aureus strains BP_001, BP_055 and BP_076. Different expression patterns are seen between plasmid-based and integrated gfp. Integrated gfp shows a sustained increase in expression throughout the assay, whereas plasmid-based gfp shows a high upregulation at 30 minutes and nearly no expression at 90 minutes The induced subculture (+) and uninduced subculture (−) for all three strains show expression induction dependency on the presence of aTc and the Pₜₑₜ-gfp expression system. As expected, BP_001 showed no expression throughout the experiment. The expression of GFP in BP_076 increased throughout the experiment, demonstrating expression from the genome of Staphylococcus aureus 502a. The expression pattern determined for BP_055 can be attributed to less than ideal experimental design; however, it did fulfill its purpose as a positive control for induction. BP_055 carries the Pₜₑₜ-gfp expression system on the plasmid pRAB11, a high-copy plasmid. Each plasmid has two TetR protein binding sites, which repress expression of GFP in the absence of aTc. Within 30 minutes of induction the high number of plasmids multiplied by cell count resulted in a ca. 1300 fold upregulation in GFP expression, confirming aTc was in an active form during the assay. One might expect that the expression level of GFP would be even higher at 90 minutes, but the data shows nearly no expression (ca. 7 fold upregulation compared to t=0). This is not surprising given the total number of TetR proteins present in the culture at t=90 minutes. The amount of aTc was not enough to inhibit repression by TetR at the 90-minute timepoint, resulting in nearly no expression. Gene expression from a molecularly modified strain of Staphylococcus aureus 502a was confirmed by qPCR analysis of tetracycline induced GFP expression.

### Example 20. Candidate Serum Responsive Promoters Screened by RNA Seq to Detect Up-Regulation

In this experiment, RNA sequencing of 502a Staphylococcus aureus variant strain BP_001 WT when grown in human serum compared to TSB was performed in order to gain a holistic understanding of the transcriptional changes that occur within the microorganism upon entry into the circulatory system. RNA sequencing was performed on samples collected from laboratory growth medium and human serum.

A culturing (growth assay) in TSB with or without human serum was performed as follows. S. aureus 502a cells were struck out from a cryo stock on a tryptic soy broth (TSB) agar plate with 5% sheep's blood and grown overnight (37° C.). The following day five single colonies were used to inoculate 5 mL of TSB in a 14 mL culture tube and grown overnight with agitation (37° C., 240 rpm). The next morning 50 mL of TSB were transferred to a 250 mL flask and warmed to 37° C. The OD₆₀₀ of the overnight culture was measured (OD₆₀₀=6.0) and used to inoculate (416 μL) the warmed TSB to an OD₆₀₀ of 0.05. This culture grew for ca. two hours (37° C., 100 rpm) and reached an OD₆₀₀ of 1.24. During this time a 50 mL aliquot of human serum was placed in the 37° C. incubator to thaw and warm, fresh TSB was also warmed. Using a serological pipette, 15 mL of culture were transferred to a 15 mL Falcon tube and centrifuged (RT, 2000×g, 10 min). The supernatant was decanted, the pellet was resuspended in sterile PBS (15 mL) and centrifuged (RT, 2000×g, 10 min). The supernatant from the wash step was decanted and the pellet was resuspended in sterile PBS (7.5 mL), doubling the OD₆₀₀ of the inoculum to 2.48. The PBS suspension was used to inoculate the TSB and serum culture samples at an OD₆₀₀ of 0.05 (202 μL per 10 mL medium).

RNA sequencing sample preparation was performed as follows.

The t=0 min samples (3×) were each 1 mL of the original 50 mL starter culture prior to washing. At the allotted timepoint, the culture tubes were removed from the incubator and placed in an ice water bath for 5 minutes and then centrifuged (4° C., 2000×g, 10 min). The supernatant was decanted, the pellet was resuspended in 1 mL ice-cold sterile PBS and transferred to microtubes. The suspensions were centrifuged (4° C., 6000×g, 3 min), the supernatant was aspirated off and the pellets were resuspended in RNAlater. The RNAlater suspensions were stored at −20° C.

The samples were removed from the −20° C. freezer for RNA extraction and allowed to thaw at RT. The cells were pelleted (RT, 16000×g, 1 min), the supernatant was aspirated off and the cells were then washed with PBS—washing helped remove carryover from the serum. To wash the cells, the pellets were resuspended in PBS and centrifuged (RT, 16000×g, 1 min), the supernatant was discarded. The RNA was extracted using Invitrogen's RiboPure Bacteria Kit following the manufacturer's instructions. The extracted RNA was then DNase I treated and ethanol precipitated. Per the sequencing firm's request the samples were sent as pellets in ethanol on dry ice.

From the total RNA samples, the ribosomal RNA molecules were depleted using the Ribo-Zero rRNA Removal Kit for Bacteria (Illumina). The quality of the RNA samples was analyzed on a Shimadzu MultiNA microchip electrophoresis system and then fragmented using ultrasound (4 pulses, 30 s, 4° C.). An adapter was ligated to the 3′ end of the molecules to enable first strand cDNA synthesis with M-MLV reverse transcriptase. The cDNA was purified and a 5′ Illumina TruSeq adapter ligated to the 3′ end of the antisense cDNA. The cDNA was then amplified by PCR using a high fidelity polymerase, the concentration after amplification was 10-20 ng/μL. The cDNA samples were then barcoded according to the growth condition they represented, purified using a Agencourt AMPure XP kit (Beckman Coulter Genomics) and analyzed by capillary electrophoresis. The cDNA was then pooled, the pool covered 200 to 500 bp molecules.

For Illumina NextSeq the primers used for PCR amplification were designed for TruSeq sequencing following Illumina's instructions. The cDNA was sequenced on an Illumina NextSeq 500 system using 75 bp read length. The differential expression of genes was analyzed via DESeq2 using SARTools.

Results for upregulated genes by RNA sequencing are shown in the Table 40; t=time in minutes after exposure to human serum.

TABLE 40
Genes in Staphylococcus aureus 502a WT upregulated
upon exposure to human serum by RNAseq

| | | t = 30 | t = 30 | t = 90 | t = 90 |
|---|---|---|---|---|---|
| | | Serum vs | Serum vs | Serum vs | Serum vs |
| Gene | | t = 0 | t = 30 TSB | t = 0 | t = 90 TSB |
| gene name | gene number | fold change | fold change | fold change | fold change |
| isdB | CH52_00245 | 479.653 | 471.648 | 2052.474 | 1240.112 |
| sbnB | CH52_05135 | 158.756 | 44.41 | 310.08 | 130.622 |
| isdC | CH52_00235 | 93.006 | 56.211 | 173.376 | 149.117 |
| sbnA | CH52_05140 | 88.832 | 37.808 | 143.558 | 93.474 |
| srtB | CH52_00215 | 73.135 | 47.421 | 143.059 | 170.578 |
| sbnE | CH52_05120 | 70.475 | 50.083 | 190.255 | 171.279 |
| sbnD | CH52_05125 | 66.84 | 52.434 | 187.025 | 224.017 |
| isdI | CH52_00210 | 65.951 | 53.426 | 115.302 | 118.724 |
| heme ABC | CH52_00225 | 65.024 | 43.415 | 117.603 | 135.956 |
| transporter 2 | | | | | |
| sbnC | CH52_05130 | 63.092 | 51.306 | 162.927 | 147.385 |
| heme ABC | CH52_00230 | 60.967 | 40.137 | 125.227 | 196.142 |
| transporter | | | | | |
| isd ORF3 | CH52_00220 | 51.262 | 35.978 | 97.439 | 119.584 |
| sbnF | CH52_05115 | 43.997 | 44.31 | 129.516 | 127.889 |
| alanine | CH52_11875 | 43.589 | 20.237 | 304.444 | NA |
| dehydrogenase | | | | | |
| HarA | CH52_10455 | 43.215 | 28.041 | 114.425 | 117.787 |
| sbnG | CH52_05110 | 42.446 | 34.095 | 133.373 | 120.433 |
| diaminopimelate | CH52_05105 | 32.541 | 25.864 | 102.838 | 141.629 |
| decarboxylase | | | | | |
| iron ABC | CH52_05145 | 31.417 | 19.576 | 44.885 | 47.226 |
| transporter | | | | | |
| threonine | CH52_11880 | 24.559 | 20.237 | NA | NA |
| dehydratase | | | | | |
| isdA | CH52_00240 | 21.471 | 40.712 | 44.477 | 115.432 |
| siderophore | CH52_05150 | NA | NA | 33.201 | 37.267 |
| ABC transporter | | | | | |
| sbnI | CH52_05100 | NA | 22.602 | 101.548 | 89.778 |
| SAM dep | CH52_04385 | NA | NA | 75.292 | 25.847 |
| Metrans | | | | | |

Several genes were found to be upregulated greater than 20-fold after exposure to human serum at t=30 min compared to t=0, or compared to t=30 in TSB, by RNA sequencing including isdB, sbnB, isdC, sbnA, srtB, sbnE, sbnD, isdI, heme ABC transporter 2, heme ABC transporter 2, heme ABC transporter, isd ORF3, sbnF, alanine dehydrogenase, HarA, sbnG, diaminopimelate decarboxylase, iron ABC transporter, threonine dehydratase, isdA, and sbnI.

Several genes were upregulated greater than 50-fold after exposure to human serum at t=30 min compared to t=0, or compared to t=30 in TSB, by RNA sequencing including isdB, sbnB, isdC, sbnA, srtB, sbnE, sbnD, isdI, heme ABC transporter 2, heme ABC transporter 2, heme ABC transporter, isd ORF3. Genes upregulated greater than 100-fold after exposure to human serum at t=30 miv compared to t=0, or compared to t=30 in TSB, by RNA sequencing include isdB, and sbnB,

Several genes were upregulated greater than 100-fold after exposure to human serum at t=90 min compared to t=0, or compared to t=90 in TSB, by RNA sequencing including isdB, sbnB, isdC, sbnA, srtB, sbnE, sbnD, isdI, heme ABC transporter 2, heme ABC transporter 2, heme ABC transporter, isd ORF3, sbnF, alanine dehydrogenase, HarA, sbnG, diaminopimelate decarboxylase, isdA.

Preferred upregulated genes in Staphylococcus aureus 502a when exposed to serum include isdB gene CH52_00245, srtB gene CH52_00215, heme ABC transporter2 gene CH52_00215, and HarA gene CH52_00215.

Several Staphylococcus aureus 502a WT genes were found to be downregulated when exposed to human serum by RNA sequencing as shown in Table 41 and Table 42.

TABLE 41
Genes in Staphylococcus aureus 502a WT downregulated
upon exposure to human serum at 30 min by RNAseq

| | | | t = 30 |
|---|---|---|---|
| | | t = 30 | Serum vs |
| | | Serum vs | t = 30 |
| | | t = 0 | TSB |
| | | fold | fold |
| gene name | gene number | change | change |
| phosphoribosylglycinamide | CH52_00525 | −4.307 | −2.001 |
| formyltransferase | | | |
| phosphoribosylaminoimidazole | CH52_00530 | −4.271 | −2.063 |
| synthetase | | | |
| amidophosphoribosyltransferase | CH52_00535 | −4.131 | −2.117 |
| phosphoribosylformyl- | CH52_00540 | −4.046 | −2.244 |
| glycinamidine synthase | | | |
| phosphoribosylformyl- | CH52_00545 | −3.498 | −2.215 |
| glycinamidine synthase | | | |
| phosphoribosylaminoimidazole- | CH52_00555 | −3.345 | −2.134 |
| succinocarboxamide | | | |
| trehalose permease IIC | CH52_03480 | −3.338 | −2.401 |
| DeoR faimly transcriptional | CH52_02275 | −2.55 | −2.171 |
| regulator | | | |
| phosphofructokinase | CH52_02270 | −2.464 | −1.984 |
| PTS fructose transporter | CH52_02265 | −2.042 | −1.806 |
| subunit IIC | | | |
| galactose-6-phosphate isomerase | CH52_07975 | NA | −2.137 |

TABLE 42
Genes in Staphylococcus aureus 502a WT downregulated
upon exposure to human serum at 90 min by RNAseq

| | | t = 90 Serum vs t = 0 | t = 90 Serum vs t = 90 TSB |
|---|---|---|---|
| gene name | gene number | fold change | fold change |
| NarZ | CH52_07000 | −5.012 | −3.989 |
| phosphoribosylglycinamide | CH52_00525 | −3.737 | −1.680 |
| formyltransferase | | | |
| trehalose permease IIC | CH52_03480 | −3.279 | −4.381 |
| NarH | CH52_07005 | −3.265 | NA |
| alkylhydroperoxidase | CH52_06615 | −3.211 | −3.573 |
| NarT | CH52_07045 | −3.108 | −3.680 |
| hypothetical protein | CH52_04875 | −2.911 | −3.396 |
| DeoR trans factor | CH52_02275 | −2.245 | −3.322 |
| PTS fructose transporter | CH52_02265 | −2.211 | −4.474 |
| subunit IIC | | | |
| lysophospholipase | CH52_02680 | −1.837 | −3.000 |
| protein disaggregation | CH52_01005 | −0.009 | −2.989 |
| chaperon | | | |
| alkylhydroperoxidase | CH52_06615 | NA | −3.573 |
| phosphofructokinase | CH52_02270 | NA | −3.878 |

Several genes in Staphylococcus aureus 502a were downregulated at least 2 fold after t=30 or t=90 minutes in serum compared to t=0 or in TSB including phosphoribosylglycinamide formyltransferase gene CH52_00525, trehalose permease IIC gene CH52_03480, DeoR family transcriptional regulator gene CH52_02275, phosphofructokinase gene CH52_02270, and PTS fructose transporter subunit IIC gene CH52_02265.

### Example 21. Kill Switch Construction

For this experiment, a serum responsive kill switch cassette was designed and constructed for the purpose of making a strain of Staphylococcus aureus (SA) 502a that is unable to grow in serum or blood. We based this cassette around the endogenous sprA1 toxin antitoxin system in SA. This is a type I T/AT system where the toxin is a small membrane porin peptide (PepA1) that is translationally repressed by an antisense RNA. The antisense RNA binds to the 5′ UTR of sprA1 covering the RBS and blocking its ability to bind to the single stranded mRNA and synthesize the protein.

The design of this kill switch changes the promoter region that drives the expression of the PepA1 toxin from its endogenous system to one that is highly upregulated when the organism is cultured in human serum. This construct was made with the sbnA promoter from SA 502a. For this kill switch, the promoter region was not changed for the antisense RNA, but additional versions of kill switches are in progress that will have this region changed as well to promoters that have been identified to be highly upregulated during growth in normal complex media, but highly repressed or down regulated when the organism is grown in blood or serum. This should make it even easier to overcome the antitoxin suppression of sprA1 in blood or serum conditions.

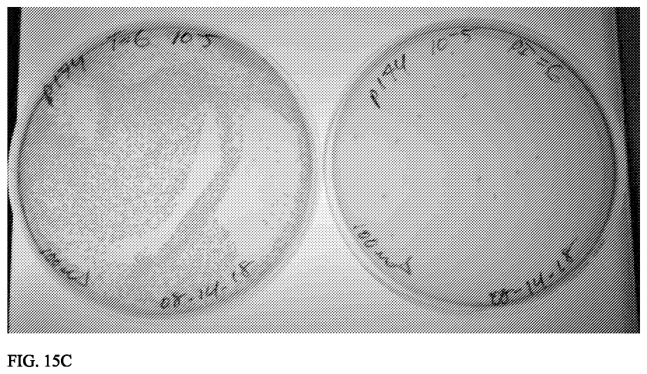

To test the functionality of the kill switch, the expression of the PepA1 toxin was induced by taking a culture that was growing at early exponential phase in complex media, tryptic soy broth (TSB), and changing the growth media to human serum. The OD was monitored and serial dilutions to plate were performed and CFUs were counted to monitor the number of viable cells in the culture and compare it to wild type SA 502a grown under the same conditions. FIG. 21 shows a map of PsbnA-sprA1 Kill Switch in Staphylococcus aureus 502a genome.

The methods used for plasmid construction, oligos, protocol for making changes in Staphylococcus aureus 502a genome using homologous recombination, and Kill Assay are shown below.

Strains

502a—Staphylococcus aureus wild type

BP_011-502a ΔsprA1-sprA1(AS)

BP_084-502a ΔPsprA::PsbnA

In this experiment BP_011 has both the sprA1 toxin gene and sprA1 antitoxin region knocked out, because it was considered to be easier to “cure” the KO by integrating the kill switch into that site than to do the integration directly into the wild type 502a. This is because the system used for integrations, i.e. homologous recombination, relies on segments of homology between the inserted gene and the chromosomal target to dictate the location of the integration, and it was felt the endogenous sprA1 toxin/antitoxin might interfere with the integration if present in the genome. The BP_011 strain is the parent of the kill switch strain BP_084. The BP-011 strain was included in this experiment as a control.

Plasmid Construction
1) PCR amplify homology regions from SA 502a genome
   a. Upstream Homology Arm—DR_233/DR_296
   b. Downstream Homology Arm—DR_280/DR_236
2) PCR amplify PsbnA-sprA1 from synthesized linear DNA fragment from IDT
   a. PsbnA-sprA1—DR_297/DR_228
3) PCR amplify pIMAYz backbone vector
   a. DR_022/DR_023
4) Gel purify all fragments with Qiagen kit per manufactures instructions
5) Assemble linear DNA fragments into circular plasmid and transform into electrocompetent IM08B E. coli cells per the manufacturer's instructions
6) Perform colony PCR to screen colonies for fully assembled plasmid
   a. DR_117/DR_228 (1571 bp fragment)
7) Pick multiple positive colonies, grow culture overnight and sequence the plasmid to confirm there are no mutations in the newly assembled plasmid
8) Transform sequence confirmed plasmid into electrocompetent SA 502a and follow protocol for making edits in SA genome using homologous recombination
9) Screen final colonies by PCR for integrant with the primer pair DR_303/DR_304
   a. Send PCR product for sequence confirmation if correct band size is observed.

TABLE 43
Oligo Sequences used in plasmid construction

| Primer | |
|---|---|
| Name | 5′-3′ DNA sequence |
| DR_233 | cgacggtatcgataagatgGCCACTGGCGTCAAATACTGTAA |
| | TGAAGAATG (SEQ ID NO: 330) |
| DR_296 | CATCTAATTGGACAAATTCTATGAGAGTAGATTTTGTTAATT |
| | TAAG (SEQ ID NO: 331) |
| DR_280 | GTAGACGCAATACAAAATAGGTGACATATAGCCGCACC |
| | (SEQ ID NO: 332) |
| DR_236 | gctctagaactagtggatcccGGCGATTTTATTGTGACAAGA |
| | GACTGAAGAGC (SEQ ID NO: 333) |
| DR_297 | CATAGAATTTGTCCAATTAGATGTCCCACTACATCCTGCTAA |
| | AACAAGTAGGAAAGC (SEQ ID NO: 334) |
| DR_228 | CTATTTTGTATTGCGTCTACTTAGCCAATAAG |
| | (SEQ ID NO: 335) |
| DR_022 | Caagcttatcgataccgtcgacctc |
| | (SEQ ID NO: 336) |
| DR_023 | Gggatccactagttctagagcgg |
| | (SEQ ID NO: 337) |
| DR_303 | CAAGCCACCAAAGCACGTGCCTATTTGCC |
| | (SEQ ID NO: 338) |
| DR_304 | CAGTGAAATAGATAGATTGGTTGAAAAACAATCTTCAAAAGT |
| | CGGACG (SEQ ID NO: 339) |

The protocol used for making changes in Staphylococcus aureus 502a genome using homologous recombination is shown below.

### Materials

BHI agar (Chloramphenicol 10 ug/mL) (X-Gal 100 ug/mL)
BHI agar (AnhydroTet 1 ug/mL) (X-Gal 100 ug/mL)
BHI agar
BHI broth
Primers to screen colonies after primary and secondary recombination events

### Protocol

1. Prepare a highly concentrated pIMAYz integration plasmid. ˜25 mL overnight culture spun down into (4) 2× volumes of the miniprep protocol. This can be purified through 2 columns if desired, and performed to maximize yield of DNA. Elutions should be pooled and concentrated using the Zymo concentrator kit performed to maximize concentration.
2. Use up to 5 uL of concentrated plasmid from above to transform 502a using the labs optimized electroporation protocol.
3. Recover cells for 1 hr at 30° C. in shaker
4. Plate entire recovery mixture between 3-4 BHI (Chlor 10, X-Gal 100) agar plates. Incubate 1 plate at 30° C. and the rest at 37° C. overnight (make sure incubator is at 37 C or above)
5. Screen blue colonies on the plates for the presence of circular plasmid using primers DR_116/DR_117. The primers are flanking the multiple cloning site in pIMAYz, and for the 30 C plates will produce a band the same size as the homology arms plus any region being integrated. The 37° C. plates should not produce any band.
6. The blue colonies on the 37° C. plates should be screened for the integrated plasmid into the genome using primers that bind outside the homology arms. Each primer should be paired with either DR_116 or DR_117. This will confirm that the plasmid is integrated into the proper location in the genome.
7. If no colonies on the 37° C. plates produce bands indicating the plasmid has been integrated, colonies showing a plasmid band on the 30° C. plates can be diluted and plated on BHI agar (Chlor 10, X-Gal 100) and incubated at 37° C. Repeat steps 5-6 to rescreen the new colonies for integration.
8. If PCR shows integrated plasmid, pick a couple colonies, if possible pick clones that have integrated each way. Grow overnight (˜16 hr) in 5 mL BHI broth in room temp shaker.
9. Dilute to 10\hat{ }-5 and 10\hat{ }-6 and plate 50 uL on BHI agar (AnhydroTet 1 ug/mL, X-Gal 100 ug/mL). Incubate plates overnight at 37° C.
10. Patch white colonies to BHI agar (Chlor 10 uG/mL, X-Gal 100), BHI agar (AnhydroTet 1 ug/mL, X-Gal 100 ug/mL), BHI agar to screen for resistance to anhydrotet and sensitivity to chloramphenicol. Colonies with both phenotypes should be picked from the BHI agar plate and screened for the knock out or knock in. At least one of the primers used to screen the final genotype should bind outside the regions used as homology arms.
11. Streak plate from patch plate of several positive clones, perform HF PCR using primers that bind outside the homology arms, and send for sequencing. Incubate plates overnight at 37° C.
12. Pick at least 3 colonies from struck out plates and perform colony PCR to confirm genotype. If PCR's are all positive, the plate is used to create strain stocks and a new strain number is assigned.

The kill assay used for preliminary evaluation of the synthetic PsbnA-sprA1 Kill Switch in Staphylococcus aureus 502a genome is shown below.

Kill Assay
1) Start 5 mL TSB cultures of strains to be tested and wild type control strain and grow overnight at 37° C. in an incubator with orbital shaking at 250 RPM
2) The following day perform 1:100 dilutions into fresh TSB media and allow the cultures to grow for 2 hours.
3) Take an OD600 reading and record the values. Calculate the volume of cell culture required to inoculate 5 mL cultures to an OD of 0.05. Inoculate new cultures with calculated volume into prewarmed media (TSB/serum)
4) Continue to grow cultures at 37° C. Perform serial dilutions and plate several cell dilutions on BHI or TSB agar plates. Incubate the plates overnight at 37° C. and count the colonies on each plate after they appear (>16 hr).

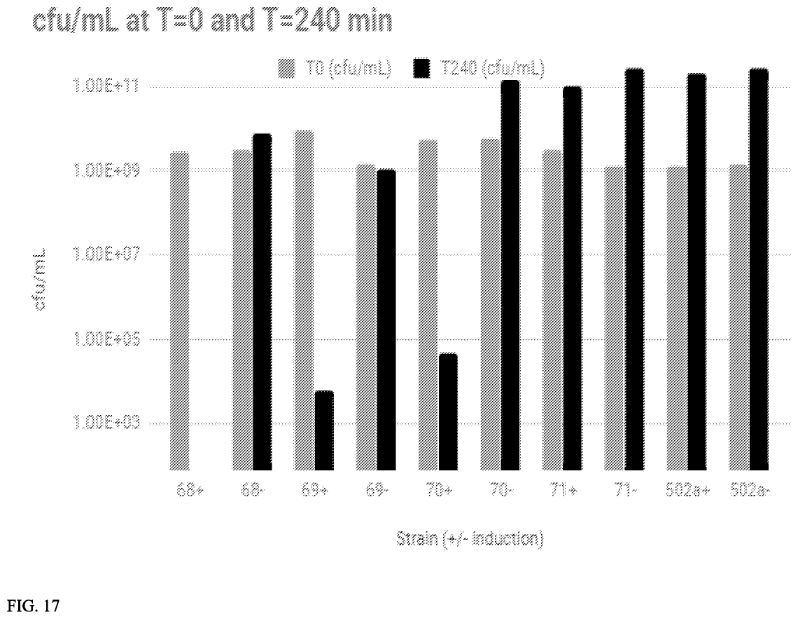

Preliminary results using PsbnA-sprA1 Kill Switch in Staphylococcus aureus 502a genome showed there was no difference in growth curves between KS and wild-type under normal growth conditions in TSB, as desired. Recorded colony counts are shown in Table 44 and FIG. 23.

TABLE 44
Recorded colony counts after 180
min when exposed to human serum

| | | Recorded Colony Count | | | |
|---|---|---|---|---|---|
| Strain | t = min | t = 0 min | t = 45 min | t = 90 min | t = 180 min |
| BP_011 | TSB | 188*10\hat{ }4 | 409*10\hat{ }4 | 30*10\hat{ }6 | 68*10\hat{ }7 |
| | Serum | 560*10\hat{ }3 | 76 * 10\hat{ }4 | 63*10\hat{ }5 | 5*10\hat{ }7 |
| 502a | TSB | | 305*10\hat{ }4 | 199*10\hat{ }5 | 89*10\hat{ }6 |
| | Serum | | 305*10\hat{ }4 | 35*10\hat{ }5 | 6*10\hat{ }7 |
| BP_084 | TSB | 220*10\hat{ }4 | 75*10\hat{ }5 | 77*10\hat{ }5 | 135*10\hat{ }6 |
| | Serum | 62*10\hat{ }4 | 180*10\hat{ }4 | 34*10\hat{ }5 | 157*10\hat{ }4 |

As shown in Table 44 an FIG. 23, after three hours of exposure to human serum, the Staphylococcus aureus KS strain BP_084 having the kill switch incorporated to the genome had fewer colonies than the wild-type strain by a factor of about 1000.

The calculated cfu/ml was found by taking the number of colonies counted*dilution factor*20 (to account for 50 uL being plated from each dilution) as shown in Table 45.

TABLE 45
Calculated cfu/mL in Human Serum and TSB

| | | Calculated CFU/mL | | | |
|---|---|---|---|---|---|
| Strain | t = min | 0 | 45 | 90 | 180 |
| BP_011 | TSB | 37600000 | 81800000 | 600000000 | 13600000000 |
| | Serum | 11200000 | 15200000 | 126000000 | 1000000000 |
| 502a | TSB | | 61000000 | 398000000 | 1780000000 |
| | Serum | | 61000000 | 70000000 | 1200000000 |
| BP_084 | TSB | 44000000 | 150000000 | 154000000 | 2700000000 |
| | Serum | 12400000 | 36000000 | 68000000 | 31400000 |

Using the data in Table 45, the cfu/mL of the kill switch strain was compared to wild type 502a. After 3 hours post serum induction, the strain harboring the integrated kill switch Staphylococcus aureus KS strain BP_084 (502a ΔPsprA::PsbnA) showed a survival rate of 2.61%, which corresponds to a 97.39% reduction in viable cells compared to the wild type in serum.

Also as shown in Table 45 after three hours of exposure to human serum, the Staphylococcus aureus KS strain BP_084 having the kill switch incorporated to the genome exhibited the survival percentage of BP_084(serum)/BP_084(TSB)*100=1.16% survival percentage. Therefore, when exposed to human serum the Staphylococcus aureus KS strain BP_084 (502a ΔPsprA::PsbnA) cells at 3 hours post-induction exhibited 100%-1.16%=98.84% measurable average cell death compared to the same BP_084 cells in TSB.

The synthetic microorganism BP_084 comprising the kill switch molecular modification incorporated to the genome exhibited desired growth properties under normal conditions, but significantly reduced cell growth when exposed to human serum.

### Example 22. Kill Switch Construction with Expression Clamp

Kill switch construction with expression clamp will be performed as follows. In prior examples, certain genes were identified that are up or down regulated in Staphylococcus aureus when exposed to human serum and blood. For example, isdB is selected as a promoter that is significantly upregulated a blood and serum responsive promoter. Also, clfB is selected as a second promoter for use in an expression clamp that is active in the absence of serum or blood, but is downregulated in the presence of serum or blood.

In prior examples, an endogenous toxin in Staph aureus was identified that when significantly upregulated, kill the cell. For example, sprA1 toxin is selected as a cell death gene.

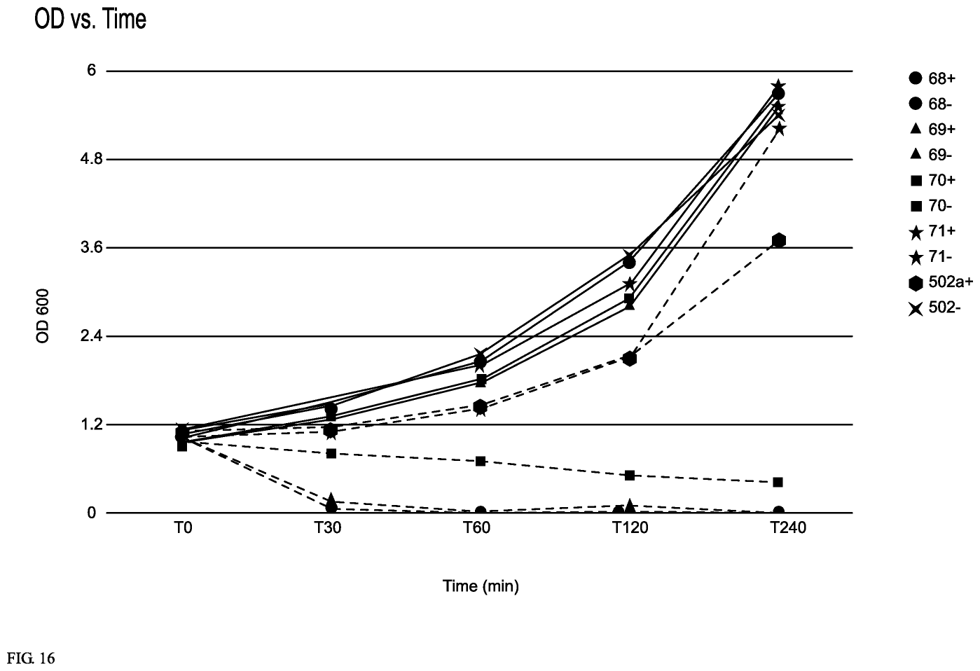

By using stitch PCR and Gibson assembly, operons are constructed that use the promoter region responsible for upregulating serum/blood genes in Staphylococcus aureus to drive the expression of the sprA1 toxin, and using the promoter regions responsible for downregulating serum/blood genes in Staphylococcus aureus to drive the expression of the sprA1_{AS}. FIG. 22 shows a cartoon of kill switch construction using serum and blood responsive promoter isdB operably linked to sprA1 cell death gene and a second promoter clfB operably linked to sprA AS to prevent leaky expression of the toxin in the absence of blood or serum. This cassette will be integrated into the native sprA1 location in the genome of Staphylococcus aureus 502a by homologous recombination, using the same technique described in previous examples.

To confirm utility, kill assay experiments will be performed using synthetic Staphylococcus aureus 502a to determine functionality of Kill Switch under various culture conditions and dermal assays in the absence and presence of blood or serum. The synthetic Staphylococcus aureus 502a will exhibit good growth under dermal or mucosal conditions, but will exhibit significantly reduced growth or cell death when exposed to blood or serum. It is contemplated that the colonized synthetic Staphylococcus aureus 502a will thus be safe to administer to a subject because it will be unable to survive or reproduce under systemic conditions. It is also contemplated that the synthetic Staphylococcus aureus 502a will be able to durably occupy a vacated niche in a host microbiome created by decolonization of a Staphylococcus aureus strain such as MRSA.

### Example 23. Kill-Switched Staphylococcus aureus Self-Destruct in Serum

After kill switch integration was confirmed via sequencing, efficacy of the kill-switched Staphylococcus aureus strain was tested by inoculating human serum with the strain and observing its growth curve by CFU/mL plating. The kill switch is intended to kill the organism in serum, but not under normal growth conditions. Therefore the kill switch strain was also grown in TSB to act as an experimental growth. The 502a wild type was also grown in serum and TSB.

Wild-type Staphylococcus aureus strain 502a and a kill-switched strain BP_088 having a S. aureus 502a base strain and isdB::sprA1 genotype were employed. The sprA1 molecular modification comprised SEQ ID NO: 284.

Protocol

### Day 1

1. Streak plate of all strains to be tested on TSB or blood agar plates from frozen glycerol stocks.

### Day 2 (PM)

1. Start overnight cultures from a single colony of KS strain and 502a in 5 mL of TSB. Incubate with agitation (37° C., 240 rpm)
   a. If running triplicate samples, pick 3 single colonies to start 3 overnight KS cultures

### Day 3: KS Assay

1. The following morning, cut back the overnight culture to 0.05 OD₆₀₀ in 5.5 mL of fresh TSB
   a. Measure the OD₆₀₀ by diluting the culture 1:10 in TSB (100 uL culture in 900 uL TSB)
   b. Calculate the necessary volume of overnight culture to inoculate fresh culture tube: (0.05*5.5)/OD600
      i. 5.5 ml is the recommended final volume
   c. Inoculate 5.5 mL of TSB and incubate the culture with agitation (37 C, 240 rpm) for 2 hrs.
2. Roughly one hour before the incubation step concludes, remove a tube of human serum from the −20° C. freezer and place in the 37° C. incubator to thaw.
3. Using a repeater pipette, fill sterile microtubes with 450 uL of sterile PBS for serial dilutions (time saver)
4. Once thawed, vortex well and transfer 5 mL of human serum using a serological pipet to a 14 mL culture tube. Repeat for number of KS cultures to be tested. Fill an equal number of culture tubes with 5 mL of TSB. Place in the 37° C. incubator to warm.
5. 2 hrs after the fresh cultures in step 1c were inoculated, measure the OD₆₀₀.
   a. Dilute 0.5 mL of cultures 1:1 in a cuvette using fresh TSB and measure OD₆₀₀
   b. Centrifuge cultures using Beckmann-Coulter centrifuge (3500 rpm, 5 mins, RT), wash in 5 mL PBS
   c. Centrifuge again and resuspend in 1 mL sterile PBS
   d. Calculate amount of resuspended culture needed to inoculate 5 ml of TSB/Serum at 0.05 OD600
   e. Inoculate 5 mL at 0.05 OD600 of prewarmed Serum and TSB from step 4.
      i. after addition of inoculum, quickly mix by pulse vortexing
6. Collect t=0 hr time point
   a. Pulse vortex culture tube (5×) and transfer 100 uL of culture using P200 pipette to prefilled microfuge tube with 900 uL sterile PBS.
   b. Repeat until all samples have been taken from culture tubes
   c. Place culture tubes into 37° C. incubator with agitation (240 rpm) and start timer
   d. Finish serial dilutions for t=0 (10⁻⁴) and plate 100 ul of 10⁻⁴ dilution on TSB plates
      i. Before each transfer, pulse vortex (5×) and during transfer aspirate/dispense (3×)
      ii. 100 uL will be transferred for each dilution
      iii. All plates are incubated at 37° C.
7. Collect remaining timepoints following dilution plating.

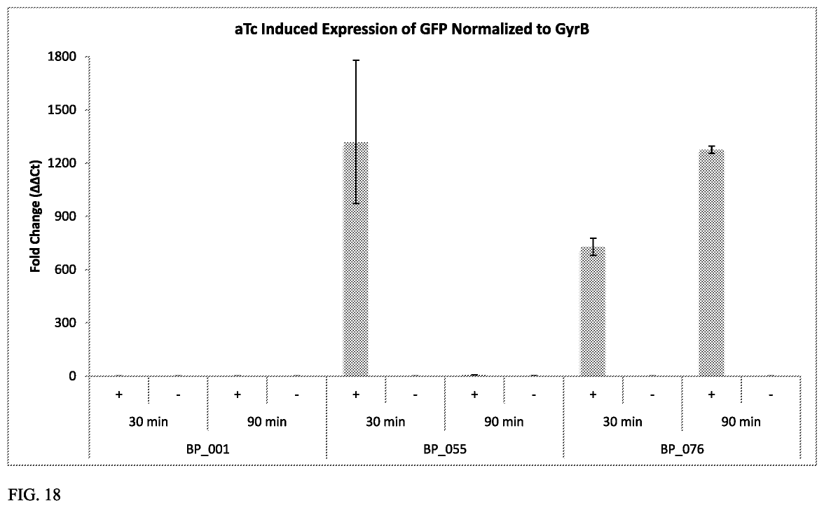

Results are shown in FIG. 24.

At time=0 hours, mean cell count for each condition were about 1×10⁵ cells. Specifically, at t=0, mean cell count for 502a cells in TSB was 8×10⁴ cells; 502a in serum was 1×10⁵ cells, BP88 cells in TSB was 7×10⁴ cells, and BP88 cells in serum was 8×10⁴ cells. Cell count was followed every 6 hours for 24 hours as shown in FIG. 24.

After 6 hours, mean cell counts for BP88 in TSB was 1×10⁸ cells indicating good growth, while mean cell count in serum dropped to no detectable cells and stayed at no detectable cells for the remainder of the 24 h assay indicating the kill switch functioned as designed to kill the synthetic cell in serum. In contrast, after 6 hours mean cell counts for wild-type 502a in TSB and serum were 2×10⁸ and 2×10⁷, respectively. After 12 hours, 502a in both serum and TSB exhibited mean cell counts at or above lethal dose level. This assay demonstrates that kill switched cells kill themselves in blood, serum, and plasma. They can colonize in the absence of blood serum or plasma, but cannot infect.

### Example 24. Mouse Tail Vein Inoculation Survival Study

A 7-day study of the clinical effectiveness of kill switched Staphylococcus aureus compared to bacteremia caused by wild-type S. aureus was performed in BALB/c mice in the tail vein injection study. Killed wild-type, live wild-type, and live kill switched Staphylococcus aureus strains were employed.

Strains included an unmodified wild-type BP0001 (502a) Staphylococcus aureus strain, a kill-switched Staphylococcus aureus BP_109 strain having a BP0001 base strain and a isdB::sprA1, PsbnA::sprA1, □spra1 genotype prepared by homologous recombination, a wild-type CX0001 isolated Staphylococcus aureus strain, and kill-switched CX_013 Staphylococcus aureus having a CX0001 base strain and a isdB::sprA1 genotype prepared by homologous recombination. The synthetic strains included one or more sprA1 molecular modifications comprising SEQ ID NO: 284.

Cultures of each strain to be tested were started in 5 mL TSB media and grown overnight at 37° C. in a shaking incubator. The following day a 1:100 dilution into 100 mL of fresh TSB media was made and the cultures were grown for another 8 hours. The cultures were then spun down by centrifugation to pellet the cells, and washed 3 times with PBS to remove any media components. 100 uL was removed and serially diluted and plated in triplicate on TSB agar plates and incubated for 12 hr at 37° C. to determine the number of cfu's present in the PBS suspension. During the incubation period the PBS cell suspension was stored at 4° C. to maintain cell viability. Once the 12 hr incubation period was up, the cfus were counted on the plates and calculations were performed to determine the number of cfus in the stock tube, which was then used to determine the volume required to get 10\hat{ }6 and 10\hat{ }7 cfu per sample to deliver for injection.

For the killed Staph aureus cells, an aliquot of 10\hat{ }6 and 10\hat{ }7 cfu of 502a was made and then incubated in 70% isopropyl alcohol for 1 hr at room temperature, then washed three times in PBS to remove residual alcohol, and brought to volume for injection. All samples were hand delivered to the CARE facility where the study was performed.

BALB/c mice were employed (n=5 each group). Prior to dosing on Day 0, baseline body weights were obtained. Morning body weights were obtained for study Days 1-6. An animal was considered moribund if 20% or greater body weight loss was noted from the baseline (Day 0) body weight along with confirmation of morbidity by clinical signs. Twice daily (AM and PM) mortality and moribundity checks were conducted.

Mice each received a 200 microliter dose of cfu dose concentration shown in Table 46. Sterile PBS was used as vehicle.

TABLE 46
Mouse tail vein injection study*

| | Mouse | Bug | NC/KS/WT | # | Day 0 | Day 1 | Day 2 | Day 3 | Day 4 | Day 5 | Day 6 | Day 7 |
|---|---|---|---|---|---|---|---|---|---|---|---|---|
| # | ID | ID | Group | Cells | Status | Status | Status | Status | Status | Status | Status | Status |
| 1 | 1001 | NA | NC PBS | NA | X | X | X | X | X | X | X | X |
| 2 | 1002 | NA | NC PBS | NA | X | X | X | X | X | X | X | X |
| 3 | 1003 | NA | NC PBS | NA | X | X | X | X | X | X | X | X |
| 4 | 1004 | NA | NC PBS | NA | X | X | X | X | X | X | X | X |
| 5 | 1005 | NA | NC PBS | NA | X | X | X | X | X | X | X | X |
| 6 | 2001 | BP001 | NC killed | 10\hat{ }6 | X | X | X | X | X | X | X | X |
| 7 | 2002 | BP001 | NC killed | 10^{\hat{ }}6 | X | X | X | X | X | X | X | X |
| 8 | 2003 | BP001 | NC killed | 10^{\hat{ }}6 | X | X | X | X | X | X | X | X |
| 9 | 2004 | BP001 | NC killed | 10^{\hat{ }}6 | X | X | X | X | X | X | X | X |
| 10 | 2005 | BP001 | NC killed | 10^{\hat{ }}6 | X | X | X | X | X | X | X | X |
| 11 | 3001 | BP001 | NC killed | 10^{\hat{ }}7 | X | X | X | X | X | X | X | X |
| 12 | 3002 | BP001 | NC killed | 10^{\hat{ }}7 | X | X | X | X | X | X | X | X |
| 13 | 3003 | BP001 | NC killed | 10^{\hat{ }}7 | X | X | X | X | X | X | X | X |
| 14 | 3004 | BP001 | NC killed | 10^{\hat{ }}7 | X | X | X | X | X | X | X | X |
| 15 | 3005 | BP001 | NC killed | 10^{\hat{ }}7 | X | X | X | X | X | X | X | X |
| 16 | 4001 | BP001 | WT | 10^{\hat{ }}6 | X | X | X | X | X | X | X | X |
| 17 | 4002 | BP001 | WT | 10^{\hat{ }}6 | X | X | X | D | D | D | D | D |
| 18 | 4003 | BP001 | WT | 10^{\hat{ }}6 | X | X | X | X | X | X | X | X |
| 19 | 4004 | BP001 | WT | 10^{\hat{ }}6 | X | X | X | D | D | D | D | D |
| 20 | 4005 | BP001 | WT | 10^{\hat{ }}6 | X | X | X | X | D | D | D | D |
| 21 | 5001 | BP001 | WT | 10^{\hat{ }}7 | X | X | X | X | D | D | D | D |
| 22 | 5002 | BP001 | WT | 10^{\hat{ }}7 | X | X | X | X | D | D | D | D |
| 23 | 5003 | BP001 | WT | 10^{\hat{ }}7 | X | X | X | D | D | D | D | D |
| 24 | 5004 | BP001 | WT | 10^{\hat{ }}7 | X | X | X | D | D | D | D | D |
| 25 | 5005 | BP001 | WT | 10^{\hat{ }}7 | X | X | X | X | X | X | D | D |
| 31 | 9001 | CX001 | WT | 10^{\hat{ }}7 | X | X | X | X | X | X | X | X |
| 32 | 9002 | CX001 | WT | 10^{\hat{ }}7 | X | X | X | X | D | D | D | D |
| 33 | 9003 | CX001 | WT | 10^{\hat{ }}7 | X | X | X | D | D | D | D | D |
| 34 | 9004 | CX001 | WT | 10^{\hat{ }}7 | X | X | X | X | X | D | D | D |
| 35 | 9005 | CX001 | WT | 10\hat{ }7 | X | X | X | X | X | X | X | X |
| 36 | 10001 | BP109 | KS | 10\hat{ }6 | X | X | X | X | X | X | X | X |
| 37 | 10002 | BP109 | KS | 10\hat{ }6 | X | X | X | X | X | X | X | X |
| 38 | 10003 | BP109 | KS | 10\hat{ }6 | X | X | X | X | X | X | X | X |
| 39 | 10004 | BP109 | KS | 10\hat{ }6 | X | X | X | X | X | X | X | X |
| 40 | 10005 | BP109 | KS | 10\hat{ }6 | X | X | X | X | X | X | X | X |
| 41 | 11001 | BP109 | KS | 10\hat{ }7 | X | X | X | X | X | X | X | X |
| 42 | 11002 | BP109 | KS | 10\hat{ }7 | X | X | X | X | X | X | X | X |
| 43 | 11003 | BP109 | KS | 10\hat{ }7 | X | X | X | X | X | X | X | X |
| 44 | 11004 | BP109 | KS | 10\hat{ }7 | X | X | X | X | X | X | X | X |
| 45 | 11005 | BP109 | KS | 10\hat{ }7 | X | X | X | X | X | X | X | X |
| 51 | 7001 | CX013 | KS | 10\hat{ }7 | X | X | X | X | X | X | X | X |
| 52 | 7002 | CX013 | KS | 10\hat{ }7 | X | X | X | X | X | X | X | X |
| 53 | 7003 | CX013 | KS | 10\hat{ }7 | X | X | X | X | X | X | X | X |
| 54 | 7004 | CX013 | KS | 10\hat{ }7 | X | X | X | X | X | X | X | X |
| 55 | 7005 | CX013 | KS | 10\hat{ }7 | X | X | X | X | X | X | X | X |

*X = alive, X = alive but sick, D = dead

As shown in Table 46, after 7 days, 11/15 mice inoculated with unmodified live wild-type strains were dead, and remaining 4/15 were very sick. In contrast, 15/15 mice inoculated with live kill-switched strains survived and were unaffected after 7 days. The kill switched SA strains were on a background of identical microorganisms, and inoculated with identical strains, merely having a kill switch molecular modification inserted. This in vivo assay demonstrates kill switched synthetic microorganisms do not infect mice after tail vein inoculation.

### Example 25. Treating Staphylococcus aureus Subclinical Mastitis

In one prophetic example, heifers are decolonized and recolonized with a live biotherapeutic composition comprising a kill switched Staphylococcus aureus to prevent Staphylococcus infections from chronically infecting udders. In another example, following milking and reserving a baseline milk sample for testing, a cow having a Staphylococcus aureus subclinical mastitis/intramammary infection is cleaned in all four quarters to remove dirt and manure, followed by a broad spectrum teat dip, for example, a povidone-iodine dip for at least 15 to 30 seconds. The teats are thoroughly cleaned, and the cow is forestripped. The cow is then inoculated in all four quarters using intramammary infusion of a kill-switched therapeutic S. aureus microorganism at about 10⁶ to 10⁸ cells in a pharmaceutically acceptable carrier. The inoculation cycle may optionally be repeated for from 1 to 6 milking cycles. The milk may be sampled and discarded for 1 or more weeks following first inoculation. The cow exhibits reduced somatic cell count after 1 week following first inoculation. The SCC may be reduced to no more than 300,000 cells/mL, 200,000 cells/mL, or preferably no more than 150,000 cells/mL.

A broad spectrum anti-mastitis composition may be employed comprising synthetic strains of Staphylococcus aureus, Streptococcus spp., and Escherichia coli each comprising a kill switch genomic modification in a pharmaceutically acceptable carrier, as an intramammary infusion, and optionally as a spray.

### Example 26. Multiple sprA1 Kill Switch Designs in Staph aureus

In this example, multiple versions of kill switches (KS) using sprA1 toxin gene were integrated behind the endogenous serum-inducible isdB gene in the genome of Staph aureus target strain BP_001. The synthetic microorganisms were evaluated for KS efficacy in the presence of human serum vs complete media (TSB). For all experimental strains tested (BP_088, BP_115, and BP_118), the phenotypic response showed a significant drop in the cfu/mL when grown in human serum versus TSB, in contrast to parent target strain WT BP_001 which exhibited good growth in both TSB and human serum. Several additional KS synthetic strains were also prepared.

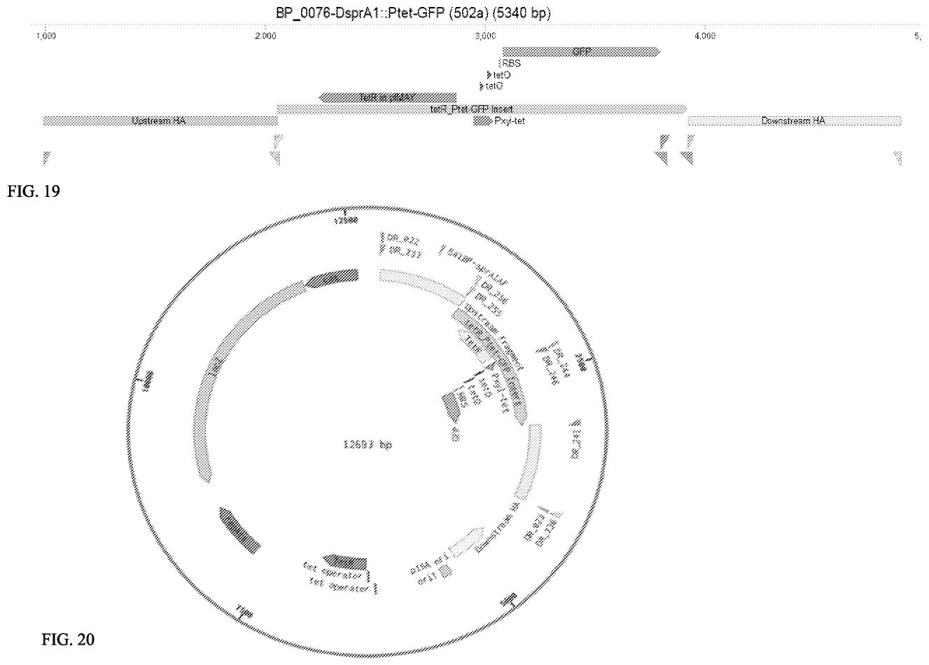

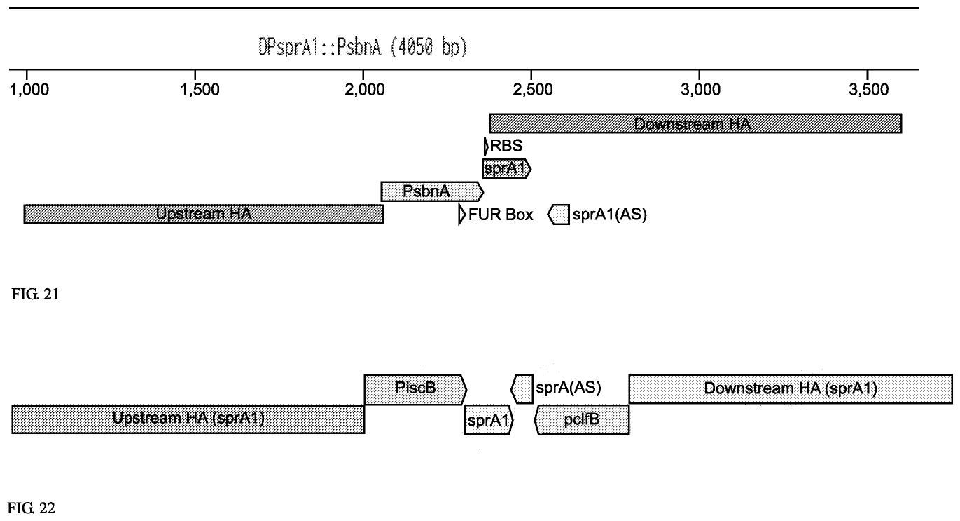

FIG. 25 shows truncated sequence alignment of the isdB::sprA1 sequences inserted to target strain BP_001 (502a) strain. The first synthetic strain BP_088 comprising isdB::sprA1 had a mutation incorporated into the upstream homology arm, which made a frame shift in the isdB gene extending the reading frame by 30 base pairs or 10 amino acids, as shown in FIG. 25(B). Despite the frame shift, BP_088 comprising isdB::sprA1 exhibited excellent suicidal cell death response (dotted lines) within 2 hours after exposure to human serum as shown in FIG. 26. BP_088 also exhibited good ability to grow in complete media (TSB, solid lines).

Additional insertion vectors were designed to investigate if the phenotypic response that was observed in serum was a result of the frame shifted isdB gene or the integrated toxin gene.

Since at first it was difficult to determine if the mutation was incorporated into the strain BP_088 due to its presence in the original insertion vector, or if the strain mutated the sequence during the recombination event in order to avoid cell death, two new vectors were prepared to test both of these options.

One of the new vectors had the same sequence as the first strain, but without the frame shift in the isdB gene and was used to prepare mutation free synthetic strain BP_118. The other new vector, used to prepare synthetic strain BP_115, added two more stop codons at the end of the isdB gene (triple stop), both in separate frames in case the strain would attempt to mutate the insert during the integration. Both of the new insertion vectors were used to make the edits in the genome of Staph aureus. The ability of synthetic strains BP_088, BP_115, and BP_118 to grow in human serum was evaluated compared to wild type Staph aureus parent strain BP_001 (502a), as shown in FIGS. 26-28.

Materials and Methods

Table 47 shows the different media and other solutions used in the experiment.

TABLE 47
Media and Other Solutions

| Name | Description | Manufacturer | Part Number |
|---|---|---|---|
| TSB | Tryptic Soy Broth (minus glucose) | Teknova | T1395 |
| TSB agar | Tryptic Soy Agar plates (minus glucose) | Teknova | T0144 |
| Human Serum | Pooled human serum | Sera care | 1830-0005 |
| PBS | 1X Phosphate buffered saline | Teknova | P0200 |

Table 48 shows the oligo names an sequences use to construct the plasmids that were used to insert the kill switches into the genome of BP_001.

TABLE 48
Oligos and Their Sequences

| Name | Sequence (5′→3′) |
|---|---|
| BP_948 | CCCTCGAGGTCGACGGTATCGATAAGCTTGGATGAGCAAGTG |
| | AAATCAGCTATTAC (SEQ ID NO: 447) |
| BP_949 | CACCTCCTCTCTGCGGATTTATTAGTTTTTACGTTTTCTAGG |
| | TAATAC (SEQ ID NO: 448) |
| BP_950 | AAAAACTAATAAATCCGCAGAGAGGAGGTGTATAAGGTGATG |
| | (SEQ ID NO: 449) |
| BP_951 | ATTAAATATAAAGACCTATTTTGTATTGCGTCTACTTAGCCA |
| | ATAAGAAAAAAAC (SEQ ID NO: 450) |
| BP_952 | CGCAATACAAAATAGGTCTTTATATTTAATTATTAAATTAAC |
| | AAATTTTAATTG (SEQ ID NO: 451) |
| BP_953 | GTGGCGGCCGCTCTAGAACTAGTGGATCCCGTCAATTACGCA |
| | ATTAAGGAAATATC (SEQ ID NO: 452) |
| DR_511 | CACCTCCTCTCTGCGCTATTCAATTAGTTTTTACGTTTTCTA |
| | GGTAATACGAATGC (SEQ ID NO: 453) |
| DR_512 | CTAATTGAATAGCGCAGAGAGGAGGTGTATAAGGTGATGC |
| | (SEQ ID NO: 454) |

Table 49 shows the plasmid genotypes use to insert the various versions of sprA1 behind the isdB gene in the genome of wild type BP_001 (502a).

TABLE 49
Plasmids Names and Function

| Plasmid Name | DNA to be Inserted Behind isdB Gene |
|---|---|
| p249 | isdB::sprA1(frame shift) |
| p260 | isdB::sprA1(triple stop) |
| p262 | isdB::sprA1 |

Table 50 shows the strains used and created in this study. The bold portion of the sequence represents the sprA1 toxin gene and the underlined sequence represents the 5′ untranslated region of the insert.

TABLE 50
Staphylococcus aureus strains

| Strain | DNA Seq. ID | Genotype | Sequence of Insert |
|---|---|---|---|
| BP_001 | n/a | 502a wild type | N/A |
| BP_088 | BP_DNA_063 | BP_001, isdB::sprA1 | ATAATAAATCCGCAGAGAGGAGGT |
| | | (frame shift) | GTATAAGGTGATGCTTATTTTCGT |
| | | | TCACATCATAGCACCAGTCATCA |
| | | | GTGGCTGTGCCATTGCGTTTTTT |
| | | | TCTTATTGGCTAAGTAGACGCAA |
| | | | TACAAAATAG (SEQ ID NO: 455) |
| BP_115 | BP_DNA_065 | BP_001, isdB::sprA1 | TTGAATAGCGCAGAGAGGAGGTGT |
| | | (triple stop) | ATAAGGTGATGCTTATTTTCGTTC |
| | | | ACATCATAGCACCAGTCATCAGT |
| | | | GGCTGTGCCATTGCGTTTTTTTC |
| | | | TTATTGGCTAAGTAGACGCAATA |
| | | | CAAAATAG (SEQ ID NO: 373) |
| BP_118 | BP_DNA_003 | BP_001, isdB::sprA1 | CGCAGAGAGGAGGTGTATAAGGTG |
| | | | ATGCTTATTTTCGTTCACATCATA |
| | | | GCACCAGTCATCAGTGGCTGTGC |
| | | | CATTGCGTTTTTTTCTTATTGGCT |
| | | | AAGTAGACGCAATACAAAATAG |
| | | | (SEQ ID NO: 342) |

All of the synthetic strains were constructed in the same manner, which is using a temperature sensitive plasmid (pIMAYz) to facilitate homologous recombination into the host's genome, and subsequent excision leaving behind the desired inserted sequence.

A protocol employing pIMAYz was designed to make edits to the genome of Staphylococcus aureus as a variation of Corvaglia et al. and Ian Monk et al. Genetic manipulation of S. aureus is difficult due to strong endogenous restriction-modification barriers that detect and degrade foreign DNA resulting in low transformation efficiency. The cells identify foreign DNA by the absence of host-specific methylation profiles. Corvaglia, A. R. et al. “A Type III-Like Restriction Endonuclease Functions As A Major Barrier To Horizontal Gene Transfer In Clinical Staphylococcus Aureus Strains”. PNAS vol 107, no. 26, 2010, pp. 11954-11958. doi:10.1073/pnas.1000489107. The E. coli strain IM08B mimics the type I adenine methylation profile of certain S. aureus strains, thus evading the endogenous DNA restriction system.

pIMAYz is an E. coli-Staph aureus shuttle vector, has a chloramphenicol resistance for both strains, and the blue/white screening technique can be used when x-gal is added to the agar plates. The plasmid is not temperature sensitive in E. coli, but is temperature sensitive in Staph aureus meaning the plasmid is able to replicate at 30° C. but is unable to do so at 37° C. The temperature sensitive feature allows for editing a target DNA sequence (genomic DNA) in vivo via homologous recombination.

The homologous recombination technique allows for markerless insertions or deletions in a target sequence using sequences that are homologous between the donor and target DNA sequences. These homologous DNA sequences (homology arms) must first be added to the plasmid backbone. Homology arms correspond to roughly 1000 bases directly upstream and downstream of the location targeted for editing. If an insertion is the end result, the DNA to be inserted should be placed in between the homology arms in the plasmid. If the end result is to be a genomic deletion, the homology arms should be right next to each other on the plasmid.

Once the plasmid is made and transformed into the target organism, the incubation temperature is raised while maintaining chloramphenicol in the media. Since the cell needs the plasmid to maintain resistance to the antibiotic, and the plasmid is unable to replicate at the higher temperatures, the only cells that survive are cells that integrated the plasmid into the target DNA (genome) by matching up the homology arms on the plasmid and target sequence. Once clones that have integrated plasmid are confirmed by PCR, a second crossover event can be allowed to happen by growing the cells with no selection pressure, then plating them on media containing anhydrotetracycline (ATc), a non-toxic analog of the antibiotic tetracycline. The ATc in the media does not directly kill the cells, but induces the secY gene on the plasmid backbone which is toxic to Staph aureus and will kill all of the cells containing the plasmid.

The cells that grow on the ATc plates have either mutated part of the secY gene, or have gone through another recombination event by matching up the homology arms on the plasmid and the genomic DNA again. The plasmid is removed through one of two routes in the second recombination event. If the same homology arms line up to remove the plasmid as did when the plasmid was integrated, there will be no change in the target DNA sequence. If the other set of homology arms line up during the second recombination event, the target molecule will either have the intended insertion or deletion. The multiple outcomes for the second event mean that colonies must be screened both genetically for the insertion/deletion, and phenotypically for their resistance to chloramphenicol and ATc. If a strain has passed all of the QC steps it can be stocked and tested to see the response of the inserted or deleted DNA.

FIG. 9 shows a diagram showing allelic exchange using pIMAY plasmid. The pIMAY plasmid can be used to make insertions in the genome of Staph aureus cells. The figure was taken from Monk et al., Mbio, vol 3, no. 2, 2012. American Society For Microbiology, doi:10.1128/mbio.00277-11.

Plasmid Construction
i. p249 (used to make BP_088) Primers for PCR amplification of homology arms and insert.
   1. Upstream homology arm
      a. BP_948/BP_949
   2. Downstream homology arm
      a. BP_952/BP_953
   3. sprA1 insert
      a. BP_950/BP_951
ii. p262 (used to make BP_118) Primers for PCR amplification of homology arms and insert.
   1. Upstream homology arm
      a. BP_948/BP_949
   2. Downstream homology arm
      a. BP_952/BP_953
   3. sprA1 insert
      a. BP_950/BP_951
iii. p260 (used to make BP_115) Primers for PCR amplification of homology arms and insert.
1. Upstream homology arm
   a. BP_948/DR_511
2. Downstream homology arm
   a. BP_952/BP_953
3. sprA1 insert
   a. DR_512/BP_951
iv. For each plasmid, the PCR amplified fragments were combined with a pIMAYz backbone vector and assembled into a circular plasmid using the Gibson Assembly Kit. per the manufacturer's instructions and transformed into electrocompetent E. coli.
v. Colonies were screened and several positive clones were sequenced to confirm proper plasmid sequence.

Strain Construction in Staph aureus
i. Sequence confirmed plasmids were transformed into electrocompetent Staph aureus and plated at 37° C. to force the integration of the plasmid.
ii. Colonies were then screened for the inserted plasmid into the genome.
   1. 3 positive clones were incubated overnight at room temp in 5 mL BHI media and plated on BHI (AtC+X-gal).
iii. White colonies were picked and screened for the presence of the plasmid both in the genome or self replicating in the cell.
iv. Colonies showing no sign of residual plasmid were screened for the inserted DNA fragment.
v. Several positive clones were sequenced to confirm the correct sequence was inserted into the genome.
vi. One sequence confirmed clone was stocked in the database and used for a serum assay.

Human Serum Assay
i. Start 3 overnight cultures from 3 separate single colonies of experimental strain in 5 mL TSB. Start one culture of 502a for internal assay control purposes and treat it in the same manner as the experimental samples.
ii. The following morning, cut back the overnight cultures to 0.05 OD600 in 5.5 mL of fresh TSB.
   1. Measure the OD600 by diluting the culture 1:10 in TSB (100 uL culture in 900 uL TSB).
   2. Calculate the necessary volume of overnight culture to inoculate fresh culture tube: (0.05*5.5)/OD600.
   3. Inoculate 5.5 mL of TSB and incubate the culture with agitation (37° C., 240 rpm) for 2 hrs to sync of the metabolism of the cells.
iii. 2 hrs after the fresh cultures in step 2 were inoculated, measure the OD600.
iv. Wash the cultures in sterile PBS.
   1. Centrifuge cultures using swing out rotor (3500 rpm, 5 mins, RT), wash with 5 mL PBS.
   2. Centrifuge again and re-suspend in 1 mL sterile PBS.
v. Calculate amount of re-suspended culture needed to inoculate 5 ml of TSB/Serum at 0.05 OD600.
vi. Inoculate (3 tubes each) of 5 mL of fresh, pre-warmed TSB and human serum at 0.05 OD600.
vii. After addition of inoculum, quickly mix by pulse vortexing and take 100 μL sample for determining cfu/mL. Place remaining cultures in 37° C. shaking incubator.
   1. Sample every two hours for the next 8 hours, and perform serial dilutions to determine cfu/mL.
      a. Serial dilutions are performed by starting with 900 μL of sterile PBS in sterile 1.5 mL tubes. A 100 μL sample is removed from a well-mixed culture and transferred into the first PBS tube.
      b. It is mixed well by pulse vortexing and 100 μL is removed and transferred to the next tube, and so on until the culture has been diluted to a point where 30-300 colonies will grow when 100 μL is spread out on a TSB agar plate. The process is repeated for all culture tubes at every time point.
      c. All plates are incubated 12-16 hours at 37° C., and the colony counts are recorded and used to calculate the cfu/mL of the cultures.

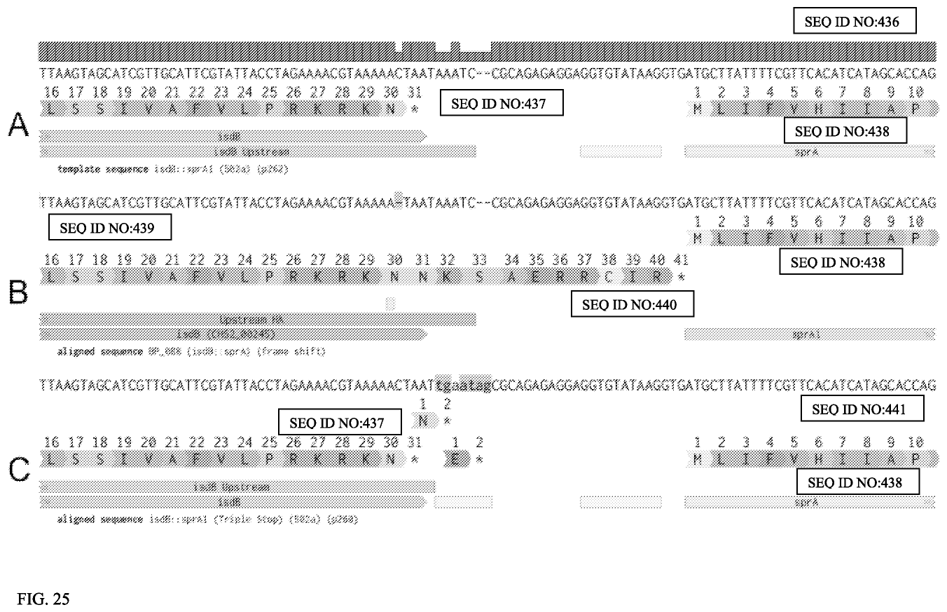

Results are shown in FIGS. 26-28 showing graphs of the colony forming units per mL of culture over 8 hours. The dashed lines represent the cultures grown in serum and solid lines represent the cultures grown in TSB. FIG. 29 shows the average (n=3) colony forming units per mL of culture over 8 hours for each of BP_088, BP_115, and BP_118 in TSB or human serum.

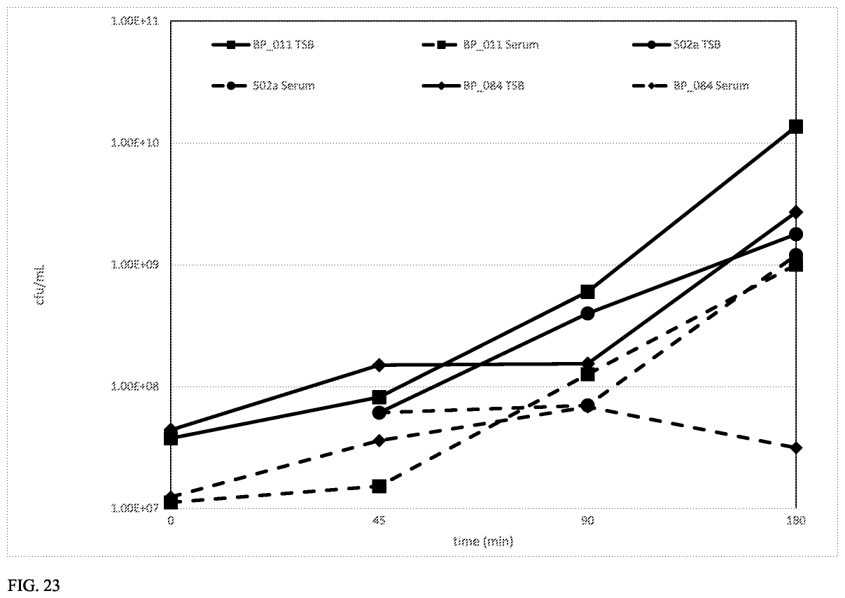

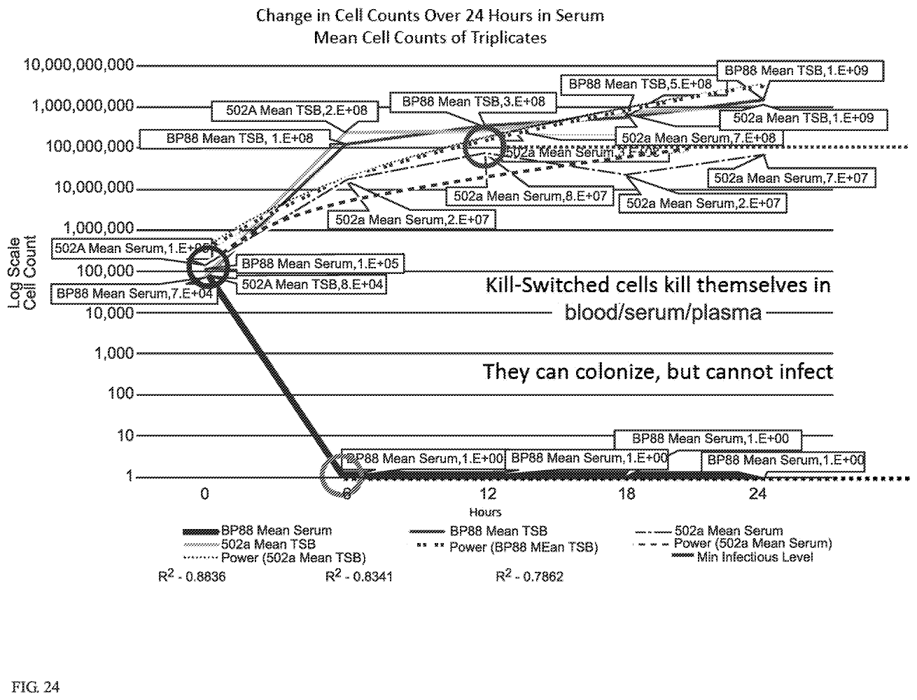

The engineered strains BP_088, BP_115, and BP_118 each comprising isdB::sprA1, and WT parent strain BP_001 each exhibited good cell growth in complete media (TSB, solid lines) as shown in FIGS. 26-28. WT BP_001 also exhibited ability to grow when exposed to human serum, as shown in FIGS. 27 and 28 (dotted lines). However, upon exposure to human serum, all three engineered strains BP_088, BP_115, and BP_118 exhibited significantly decreased growth (dotted lines) within 2 hours after exposure to human serum as shown in FIGS. 26-28.

### CONCLUSION

This series of experiments evaluated the phenotypic response of several engineered strains of Staph aureus while grown in human serum versus TSB. The strains have slightly different kill switch sequences integrated into the same location of the genome. All sequences were inserted directly behind the isdB gene.

One of the integrations resulted in the desired kill switch sequence (BP_118), another integration produced a mutation that resulted in a frame shift in the isdB gene, which is directly before the kill switch and adds 30 more bases to the isdB gene (BP_088), and the third integration introduced multiple STOP codons in different frames directly behind the isdB gene to protect the gene from being disrupted by frameshift mutations.

The three engineered strains were tested for their ability to grow in human serum and TSB versus the wild type (BP_001) strain. For all experimental strains tested (BP_088, BP_115, and BP_118), the phenotypic response showed a significant drop in the cfu/mL when grown in human serum versus TSB. This response was not observed for any WT BP_001 strains in human serum, instead that strain demonstrated the ability to grow in human serum and had multiple doublings in the same time period, whereas the other strains experienced a reduction in population of several orders of magnitude.

A number of additional kill switch cell lines were developed in a similar fashion as shown in Table 51.

TABLE 51
Kill Switch Cell Lines and Plasmids

| | | | E. coli | S. aureus |
|---|---|---|---|---|
| Plasmid | Insertion | Description | AbR* | AbR* |
| pTK001 | pCN51-Pcad-sprA1- | sprA1 kill gene and antitoxin under | Amp | Erm |
| | sprA1at | cadmium promoter | | |
| pTK002 | pCN51-Pcad-sprA1- | Reversed SprA1 kill gene and antitoxin | Amp | Erm |
| | sprA1at(rev) | under cadmium promoter | | |
| pTK003 | pCN51-PleuA-sprA1- | SprA1 kill gene and antitoxin under leuA | Amp | Erm |
| | sprA1at | promoter | | |
| pTK004 | pCN51-PleuA-sprA1- | Reversed SprA1 kill gene and antitoxin | Amp | Erm |
| | sprA1at(rev) | under leuA promoter | | |
| pTK005 | pCN51-PleuA- | SprA1 kill gene under leuA promoter, | Amp | Erm |
| | sprA1_PCLFB-sprA1at | with sprA1 antitoxin under CLFB clamp | | |
| | | promoter (opposite orientation of sprA1) | | |
| pTK006 | pCN51-PhlgA-sprA1- | SprA1 kill gene and antitoxin under hlgA | Amp | Erm |
| | sprA1at | promoter | | |
| pTK007 | pCN51-PhlgA- | SprA1 kill gene under hlgA promoter, | Amp | Erm |
| | sprA1_PCLFB-sprA1at | with sprA1 antitoxin under CLFB clamp | | |
| | | promoter (opposite orientation of sprA1) | | |
| pTK008 | pCN51-Pcad-Sma1 | Sma1 restriction enzyme kill gene under | Amp | Erm |
| | | cadmium promoter | | |
| pTK009 | pCN51-PhlgA-Sma1 | Sma1 restriction enzyme kill gene under | Amp | Erm |
| | | hlgA promoter | | |
| pTK010 | pCN51-PleuA-Sma1 | Sma1 restriction enzyme kill gene under | Amp | Erm |
| | | leuA promoter | | |
| pTK011 | pCN51-Pcad-RsaE | RsaE small RNA kill gene under | Amp | Erm |
| | | cadmium promoter | | |
| pTK012 | pCN51-PhlgA-RsaE | RsaE small RNA kill gene under hlgA | Amp | Erm |
| | | promoter | | |
| pTK013 | pCN51-PleuA-RsaE | RsaE small RNA kill gene under leuA | Amp | Erm |
| | | promoter | | |
| p080 | pCN51-Pcad-relF | relF kill gene driven by cadmium- | Amp | Erm |
| | | inducible promoter | | |
| p086 | pCN56-TT-PhlgA2- | SprA1 kill gene and antitoxin driven by | Amp | Erm |
| | sprA1-sprA1at | hlgA2 promoter | | |
| p087 | pCN56-TT-PisdG- | SprA1 kill gene and antitoxin driven by | Amp | Erm |
| | sprA1-sprA1at | isdG promoter | | |
| p088 | pCN56-TT-PsbnC- | SprA1 kill gene and antitoxin driven by | Amp | Erm |
| | sprA1-sprA1at | sbnC promoter | | |
| p089 | pCN56-TT-PsbnE- | SprA1 kill gene and antitoxin driven by | Amp | Erm |
| | sprA1-sprA1at | sbnE promoter | | |
| p090 | pCN56-TT-PhlgB- | SprA1 kill gene and antitoxin driven by | Amp | Erm |
| | sprA1-sprA1at | hlgB promoter | | |
| p091 | pCN56-TT- | SprA1 kill gene and antitoxin driven by | Amp | Erm |
| | PSAUSA300_2616- | SAUSA300_2616 promoter | | |
| | sprA1-sprA1at | | | |
| p092 | pCN56-TT-PlrgA- | SprA1 kill gene and antitoxin driven by | Amp | Erm |
| | sprA1-sprA1at | lrgA promoter | | |
| p096 | pCN56-TT-PhlgA2- | HlgA2 promoter driving sprA1 kill gene | Amp | Enn |
| | sprA1-sprA1at | and antitoxin. Promoter insert synthesized | | |
| | | and cloned into p078_pCN56-TT-sprA1- | | |
| | | sprA1at by GenScript. | | |
| p097 | pCN56-TT-Pcad- | Cadmium-inducible promoter driving | Amp | Erm |
| | sprA1-sprA1at | sprA1 kill gene and antitoxin. Promoter | | |
| | | insert synthesized and cloned into | | |
| | | p078_pCN56-TT-sprA1-sprA1at by | | |
| | | GenScript. | | |
| p098 | pCN56-TT-PhlgB- | HlgB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p099 | pCN56-TT-PsplF- | SplF promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p100 | pCN56-TT-PfhuB- | FhuB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p101 | pCN56-TT-Phlb- | Hlb promoter driving sprA1 kill gene and | Amp | Erm |
| | sprA1-sprA1at | antitoxin. Promoter insert synthesized and | | |
| | | cloned into p078_pCN56-TT-sprA1- | | |
| | | sprA1at by GenScript. | | |
| p102 | pCN56-TT-PhrtAB- | HrtAB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p103 | pCN56-TT-PisdG- | IsdG promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p104 | pCN56-TT-PlrgA- | LrgA promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p105 | pCN56-TT- | SAUSA300_2268 promoter driving | Amp | Erm |
| | PSAUSA300_2268- | sprA1 kill gene and antitoxin. Promoter | | |
| | sprA1-sprA1at | insert synthesized and cloned into | | |
| | | p078_pCN56-TT-sprA1-sprA1at by | | |
| | | GenScript. | | |
| p106 | pCN56-TT- | SAUSA200_2617 promoter driving | Amp | Erm |
| | PSAUSA300_2617- | sprA1 kill gene and antitoxin. Promoter | | |
| | sprA1-sprA1at | insert synthesized and cloned into | | |
| | | p078_pCN56-TT-sprA1-sprA1at by | | |
| | | GenScript. | | |
| p107 | pCN56-TT-PsbnE- | SbnE promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p108 | pCN56-TT-PisdI- | IsdI promoter driving sprA1 kill gene and | Amp | Erm |
| | sprA1-sprA1at | antitoxin. Promoter insert synthesized and | | |
| | | cloned into p078_pCN56-TT-sprA1- | | |
| | | sprA1at by GenScript. | | |
| p109 | pCN56-TT-PIrgB- | LrgB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p110 | pCN56-TT- | SAUSA300_2616 promoter driving | Amp | Erm |
| | PSAUSA300_2616- | sprA1 kill gene and antitoxin. Promoter | | |
| | sprA1-sprA1at | insert synthesized and cloned into | | |
| | | p178_pCN56-TT-sprA1-sprA1at by | | |
| | | GenScript. | | |
| p111 | pCN56-TT-PsbnC- | SbnC promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p133 | pIMAY-502a-2/3/5HA- | HrtAB promoter driving sprA1 kill gene | Chlor | Chlor |
| | PhrtAB-sprA1-sprA1at | and antitoxin. For genomic integration | | |
| | | into 502a via homologous recombination | | |
| | | (2/3/5 arms). | | |
| p134 | pIMAY-502a-7HA- | HrtAB promoter driving sprA1 kill gene | Chlor | Chlor |
| | PhrtAB-sprA1-sprA1at | and antitoxin. For genomic integration | | |
| | | into 502a via homologous recombination | | |
| | | (7 arms). | | |
| p135 | pIMAY-502a-2/3/5HA- | Hlb promoter driving sprA1 kill gene and | Chlor | Chlor |
| | Phlb-sprA1-sprA1at | antitoxin. For genomic integration into | | |
| | | 502a via homologous recombination | | |
| | | (2/3/5 arms). | | |
| p136 | pIMAY-502a-7HA- | Hlb promoter driving sprA1 kill gene and | Chlor | Chlor |
| | Phlb-sprA1-sprA1at | antitoxin. For genomic integration into | | |
| | | 502a via homologous recombination (7 | | |
| | | arms). | | |
| p137 | pIMAY-502a-2/3/5HA- | SbnC promoter driving sprA1 kill gene | Chlor | Chlor |
| | PsbnC-sprA1-sprA1at | and antitoxin. For genomic integration | | |
| | | into 502a via homologous recombination | | |
| | | (2/3/5 arms). | | |
| p138 | pIMAY-502a-7HA- | SbnC promoter driving sprA1 kill gene | Chlor | Chlor |
| | PsbnC-sprA1-sprA1at | and antitoxin. For genomic integration | | |
| | | into 502a via homologous recombination | | |
| | | (7 arms). | | |
| p139 | pIMAY-502a-2/3/5HA- | HlgB promoter driving sprA1 kill gene | Chlor | Chlor |
| | PhlgB-sprA1-sprA1at | and antitoxin. For genomic integration | | |
| | | into 502a via homologous recombination | | |
| | | (2/3/5 arms). | | |
| p140 | pIMAY-502a-7HA- | HlgB promoter driving sprA1 kill gene | Chlor | Chlor |
| | PhlgB-sprA1-sprA1at | and antitoxin. For genomic integration | | |
| | | into 502a via homologous recombination | | |
| | | (7 arms). | | |
| p141 | pIMAY-502a-7HA- | IsdG promoter driving sprA1 kill gene | Chlor | Chlor |
| | PisdG-sprA1-sprA1at | and antitoxin. For genomic integration | | |
| | | into 502a via homologous recombination | | |
| | | (7 arms). | | |
| p142 | pIMAY-502a-2/3/5HA- | SbnE promoter driving sprA1 kill gene | Chlor | Chlor |
| | PsbnE-sprA1-sprA1at | and antitoxin. For genomic integration | | |
| | | into 502a via homologous recombination | | |
| | | (2/3/5 arms). | | |
| p143 | pIMAY-502a-2/3/5HA- | SpIF promoter driving sprA1 kill gene | Chlor | Chlor |
| | PsplF-sprA1-sprA1at | and antitoxin. For genomic integration | | |
| | | into 502a via homologous recombination | | |
| | | (2/3/5 arms). | | |
| p144 | pIMAY-502a-2/3/5HA- | IsdI promoter driving sprA1 kill gene and | Chlor | Chlor |
| | PisdI-sprA1-sprA1at | antitoxin. For genomic integration into | | |
| | | 502a via homologous recombination | | |
| | | (2/3/5 arms). | | |
| p145 | pIMAY-502a-2/3/5HA- | SAUSA300_2616 promoter driving | Chlor | Chlor |
| | P2616-sprA1-sprA1at | sprA1 kill gene and antitoxin. For | | |
| | | genomic integration into 502a via | | |
| | | homologous recombination (2/3/5 arms). | | |
| p148 | pIMAY-502a-2/3/5HA- | LrgA promoter driving sprA1 kill gene | Chlor | Chlor |
| | PlrgA-sprA1-sprA1at | and antitoxin. For genomic integration | | |
| | | into 502a via homologous recombination | | |
| | | (2/3/5 arms). | | |
| p154 | pIMAY-502a-2/3/5HA- | HrtAB promoter driving sprG1 kill gene. | Chlor | Chlor |
| | PhrtAB-sprG1 (rev) | For genomic integration into 502a at azlC | | |
| | | locus, on sense strand, via homologous | | |
| | | recombination (2/3/5 arms). Constructed | | |
| | | by GenScript. | | |
| p155 | pIMAY-502a-2/3/5HA- | HlgA2 promoter driving 187/lysK phage | Chlor | Chlor |
| | PhlgA2-187lysK (rev) | lytic chimeric protein kill gene. For | | |
| | | genomic integration into 502a at azlC | | |
| | | locus, on sense strand, via homologous | | |
| | | recombination (2/3/5 arms). Constructed | | |
| | | by GenScript. | | |
| p156 | pIMAY-502a-2/3/5HA- | HrtAB promoter driving 187/lysK phage | Chlor | Chlor |
| | PhrtAB-187lysK (rev) | lytic chimeric protein kill gene. For | | |
| | | genomic integration into 502a at azlC | | |
| | | locus, on sense strand, via homologous | | |
| | | recombination (2/3/5 arms). Constructed | | |
| | | by GenScript. | | |
| p157 | pCN56-TT-PhlgA2- | HlgA2 promoter driving sprA1 kill gene. | Amp | Erm |
| | sprA1 | p086 with sprA1 antitoxin deleted. Kill | | |
| | | switch insert flipped orientation during | | |
| | | cloning (promoter is now closer to Staph | | |
| | | ori than E coli ori). | | |
| p158 | pCN56-TT-Phlb-sprA1 | Hlb promoter driving sprA1 kill gene. | Amp | Erm |
| | | p101 with sprA1 antitoxin deleted. | | |
| p159 | pCN56-TT-PsbnC- | SbnC promoter driving sprA1 kill gene, | Amp | Erm |
| | sprA1 | p111 with sprA1 antitoxin deleted. Kill | | |
| | | switch insert flipped orientation during | | |
| | | cloning (promoter is now closer to Staph | | |
| | | ori than E coli ori). | | |
| p160 | pIMAY-502a-9HA- | HlgA2 promoter driving sprA1 kill gene, | Chlor | Chlor |
| | PhlgA2-sprA1 | p122 with sprA1 antitoxin deleted. For | | |
| | | genomic integration into 502a via | | |
| | | homologous recombination (9 arms). | | |
| p161 | pIMAY-502a-7HA- | HrtAB promoter driving sprA1 kill gene. | Chlor | Chlor |
| | PhrtAB-sprA1 | p134 with sprA1 antitoxin deleted. For | | |
| | | genomic integration into 502a via | | |
| | | homologous recombination (7 arms). | | |
| p162 | pIMAY-502a-7HA- | Hlb promoter driving sprA1 kill gene. | Chlor | Chlor |
| | Phlb-sprA1 | p136 with sprA1 antitoxin deleted. For | | |
| | | genomic integration into 502a via | | |
| | | homologous recombination (7 arms). | | |
| p164 | pIMAY-502a-2/3/5HA- | HlgA2 promoter driving sprG1 kill gene. | Chlor | Chlor |
| | PhlgA2-sprG1 (rev) | For genomic integration into 502a at azlC | | |
| | | locus, on sense strand, via homologous | | |
| | | recombination (2/3/5 arms). Constructed | | |
| | | by GenScript. | | |
| p165 | pIMAY-502a-2/3/5HA- | HrtAB promoter driving holin kill gene. | Chlor | Chlor |
| | PhrtAB-holin (rev) | For genomic integration into 502a at azlC | | |
| | | locus, on sense strand, via homologous | | |
| | | recombination (2/3/5 arms). Constructed | | |
| | | by GenScript. | | |
| p166 | pIMAY-502a-2/3/5HA- | HlgA2 promoter driving holin kill gene. | Chlor | Chlor |
| | PhlgA2-holin (rev) | For genomic integration into 502a at azlC | | |
| | | locus, on sense strand, via homologous | | |
| | | recombination (2/3/5 arms). Constructed | | |
| | | by GenScript. | | |
| p171 | pIMAY-502a-2/3/5HA- | HlgA2 promoter driving lysostaphin kill | Chlor | Chlor |
| | PhlgA2- | gene (mature form). For genomic | | |
| | matureLysostaphin | integration into 502a at azlC locus, on | | |
| | (rev) | sense strand, via homologous | | |
| | | recombination (2/3/5 arms). Constructed | | |
| | | by GenScript. | | |
| p172 | pRAB11-Ptet-187lysK | 187/lysK phage lytic chimeric kill gene | Amp | Chlor |
| | | under control of tetracycline-inducible | | |
| | | promoter. | | |
| p173 | pRAB11-Ptet-holin | Holin kill gene under control of | Amp | Chlor |
| | | tetracycline-inducible promoter. | | |
| p174 | pRAB11-Ptet-sprA1 | SprA1 kill gene (without antitoxin | Amp | Chlor |
| | | sequence) under control of tetracycline- | | |
| | | inducible promoter. Kill gene includes | | |
| | | some sequence upstream of the start | | |
| | | codon. | | |
| p175 | pRAB11-Ptet- | sprA1 kill gene (without antitoxin | Amp | Chlor |
| | sprA1(ATG) | sequence) under control of tetracycline- | | |
| | | inducible promoter. Kill gene sequence | | |
| | | begins at start codon. | | |
| p176 | pIMAY-502a-2/3/5HA- | HlgA2 promoter driving lysostaphin kill | Chlor | Chlor |
| | PhlgA2- | gene (mature form). For genomic | | |
| | matureLysostaphin | integration into 502a at azlC locus, on | | |
| | | anti-sense strand, via homologous | | |
| | | recombination (2/3/5 arms). Constructed | | |
| | | by GenScript. | | |
| p177 | pIMAY-502a-2/3/5HA- | HrtAB promoter driving lysostaphin kill | Chlor | Chlor |
| | PhrtAB- | gene (mature form). For genomic | | |
| | matureLysostaphin | integration into 502a at azlC locus, on | | |
| | | anti-sense strand, via homologous | | |
| | | recombination (2/3/5 arms). Constructed | | |
| | | by GenScript. | | |
| p178 | pRAB11-Ptet-sprG1 | SprG1 kill gene under control of | Amp | Chlor |
| | | tetracycline-inducible promoter. | | |
| p180 | pCN56-TT-PhrtAB- | HrtAB promoter driving sprA1 kill gene, | Amp | Erm |
| | sprA1 | p102 with sprA1 antitoxin deleted. | | |
| p181 | pIMAY-502a-2/3/5HA- | HrtAB promoter driving lysostaphin kill | Chlor | Chlor |
| | PhrtAB- | gene (mature form). For genomic | | |
| | matureLysostaphin | integration into 502a at azlC locus, on | | |
| | (rev) | sense strand, via homologous | | |
| | | recombination (2/3/5 arms). Constructed | | |
| | | by GenScript. | | |
| p187 | pCN56-TT-PhlgA2- | p086 with His tag | Amp | Erm |
| | sprA1-sprA1at-His | | | |
| p188 | pCN56-TT-Pcad- | p097 with His tag | Amp | Erm |
| | sprA1-sprA1at-His | | | |
| p189 | pRAB11-Ptet-sprA1- | p174 with His tag | Amp | Chlor |
| | His | | | |
| p190 | pRAB11-Ptet- | p175 with His tag | Amp | Chlor |
| | sprA1(ATG)-His | | | |
| p196 | pRAB11-Ptet- | Lysostaphin kill gene under control of | Amp | Chlor |
| | lysostaphin | tetracycline-inducible promoter. | | |
| p232 | pCN56-TT-PhlgA2- | HlgA2 promoter driving sprA1 kill gene. | Amp | Erm |
| | sprA1 | p096 (made by GenScript) with sprA1 | | |
| | | antitoxin deleted. | | |
| p233 | pCN56_TT-P305- | Kill switch using p305 and P360 driving | Amp | Erm |
| | sprA_sprA1-P360-TT | the expression of sprA/sprA(AS) | | |
| p234 | pRAB11-Ptet-noRBS- | Tetracycline-inducible promoter driving | Amp | Chlor |
| | sprG1 | kill gene without an RBS. Serves as a | | |
| | | negative control for Ptet assays. | | |
| p235 | pCN56-TT-PhlgA2- | SprA1 kill gene and antitoxin driven by | Amp | Erm |
| | sprA1-sprA1at | hlgA2 promoter | | |
| p236 | pCN56-TT-PhlgA2- | HlgA2 promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p238 | pIMAYz_site 2::Pcad- | | Chlor | Chlor |
| | GFP | | | |
| p239 | pIMAYz_site | | Chlor | Chlor |
| | 2::PgyrB-sprA1as | | | |
| p240 | pIMAYz_site 2::Pcad- | | Chlor | Chlor |
| | sprA1 | | | |
| p241 | pIMAYz_site | | Chlor | Chlor |
| | 2::PgyrB-GFP | | | |
| p242 | pZAS_ΔPsprA1::PsbnA | | Chlor | Chlor |
| p244 | Ptet-lysostaphin | Lysostaphin kill gene under control of | | |
| | | tetracycline-inducible promoter. | | |
| p245 | Ptet-sprG1 (short) | SprG1 (short) kill gene (without antitoxin | | |
| | | sequence) under control of tetracycline- | | |
| | | inducible promoter. vector: pRAB11-Ptet | | |
| p246 | Ptet-sprA2 | SprA2 kill gene (without antitoxin | | |
| | | sequence) under control of tetracycline- | | |
| | | inducible promoter. vector: pRAB11-Ptet | | |
| p247 | Ptet-mazF | mazF kill gene (without antitoxin | | |
| | | sequence) under control of tetracycline- | | |
| | | inducible promoter. vector: pRAB11-Ptet | | |
| p248 | Ptet-YoeB-sa2 | Yoeb-sa2 kill gene (without antitoxin | | |
| | | sequence) under control of tetracycline- | | |
| | | inducible promoter. vector: pRAB11-Ptet | | |
| p249 | isdB::sprA1 | sprA with its RBS dropped in behind isdB | Chlor | Chlor |
| | | with a six base spacer. | | |
| p252 | PsbnA::sprA1 | plasmid to insert sprA1 behind the sbnA | Chlor | Chlor |
| | | promoter | | |
| p254 | 04385::sprA1 | integrates sprA1 behind CH52_04385 | Chlor | Chlor |
| p255 | 05105::sprA1 | integrates sprA1 behind CH52_05105 | Chlor | Chlor |
| p256 | 06885::sprA1 | integrates sprA1 behind CH52_06885 | Chlor | Chlor |
| p257 | 10455::sprA1 | integrates sprA1 behind CH52_10455 | Chlor | Chlor |
| p260 | isdb::sprA1(triple stop | sprA with its RBS dropped in behind isdB | Chlor | Chlor |
| | codon) | with a six base spacer, two additional stop | | |
| | | codons added after isdB in different | | |
| | | frames | | |
| p261 | isdB::sprG1 | sprG1 inserted behind isdB | Chlor | Chlor |
| p262 | isdB::sprA1 | sprA1 inserted behind isdB gene (no | Chlor | Chlor |
| | | mutations in homology arms) | | |
| p265 | PsbnA::sprG1 | sprG inserted behind PsbnA | Chlor | Chlor |
| p267 | isdB::sprA2 | sprA2 toxin behind isdB | Chlor | Chlor |
| p268 | PsbnA::sprA2 | sprA2 toxin behind sbnA promoter | Chlor | Chlor |
| | | **Note has point mutation in Right HA | | |

*AbR: Antibiotic Resistance

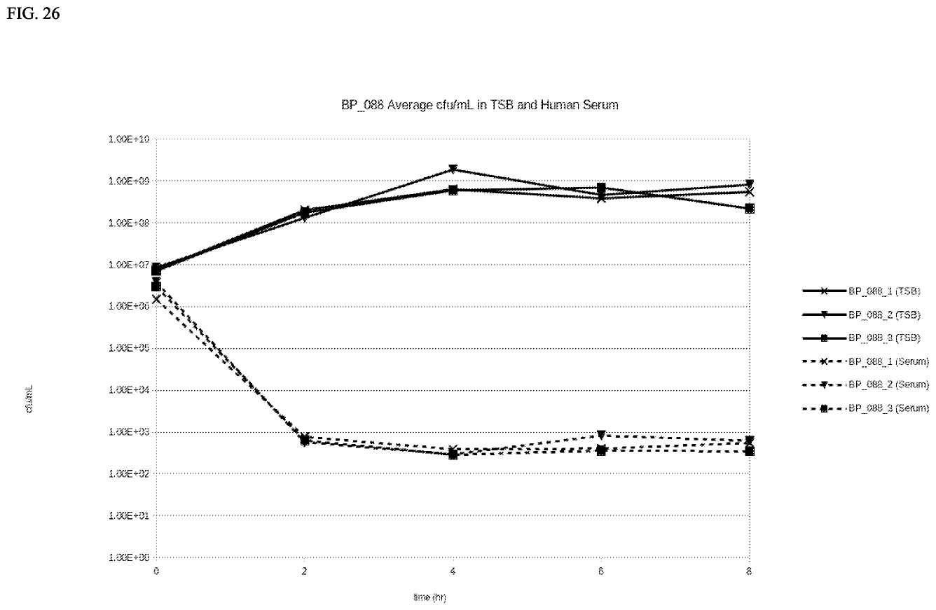

The present inventors generated a multiplicity of synthetic strains as shown in Table 52 shown in FIG. 30.

### Example 27. Truncated and Frame-Shifted sprA1 Efficacy Assay in E. coli and Staph aureus

When making the plasmid p257 (pIMAYz_harA::sprA1) the sprA1 gene acquired a base pair deletion which resulted in a frameshift and truncated protein (SEQ ID NO: 386) (BP_DNA_090) having amino acid sequence MLIFVHIIAPVISGCAIAFFLIG (BP_AA_014) (SEQ ID NO:423) A protein sequence alignment using the BLOSUM62 matrix showed a 64.5% similarity between the mutated protein and native protein having amino acid sequence (BP_AA_002) MLIFVHIIAPVISGCAIAFFSYWLSRRNTK (SEQ ID NO: 411), encoded by BP_DNA_035 (SEQ ID NO:364). In order to test the efficacy of the mutated and truncated protein the mutated sprA1 gene was inserted into the pRAB11 plasmid so it could be regulated by the P_{(xyl/tet)} promoter and induced by anhydrotetracycline (ATc). The new plasmid was named p298 and was tested in E. coli and Staph aureus BP_001 for its effect on the cell culture when overexpressed.

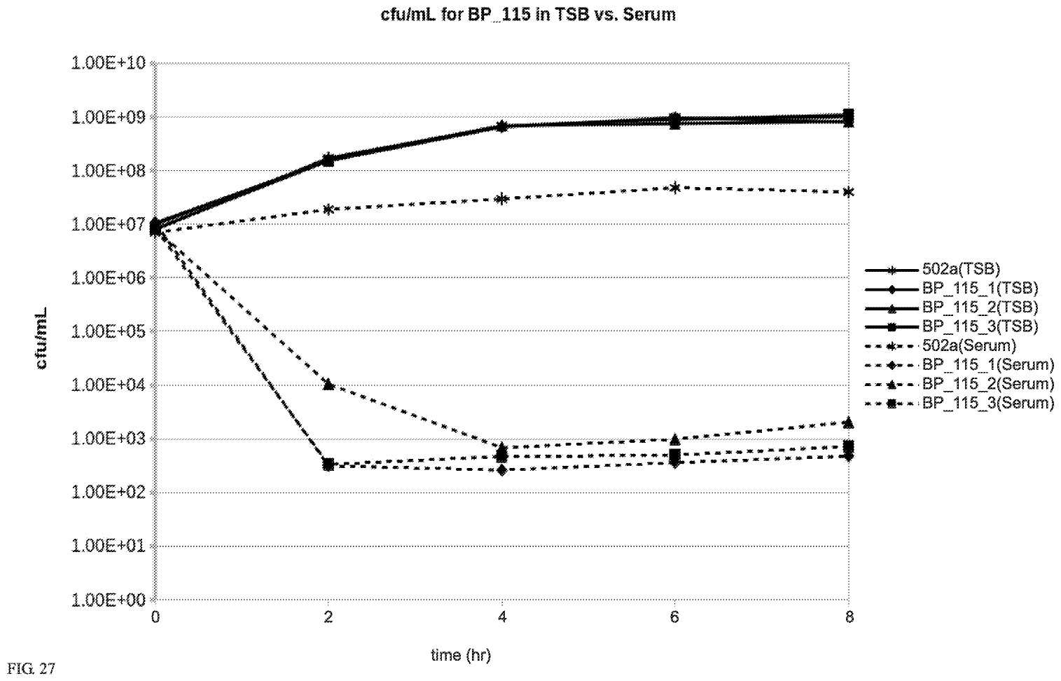

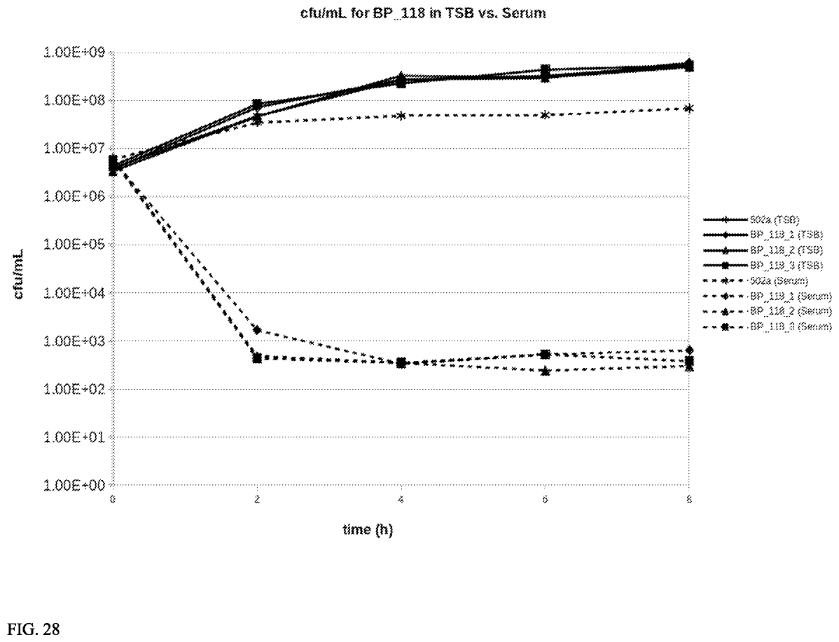

Briefly, three biological replicate overnight cultures for each strain harboring the plasmid were grown in TSB media at 37° C. in a shaking incubator at 240 rpm. The following day the cultures were cut back to an OD of 0.05 and each overnight culture was split into two tubes, grown for 2 hours at 37° C. After two hours of growth, one tube for each strain received a spike of ATc to induce the expression of the truncated sprA1 gene and then placed back in the shaking incubator to continue growing. Samples were taken every hour to measure the density of the culture by measuring the absorbance at 600 nm (OD600). FIGS. 31 and 32 show the average OD measurements plotted against time for the strains tested.

FIG. 31 shows induced and uninduced growth curves for the E. coli strain IM08B (BPEC_023) harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Within 2 hours of induction, the BPEC_023 E. coli culture growth rate slowed for each following time point and eventually went negative before the assay was stopped, whereas uninduced culture exhibited continued growth over 6 hrs of assay.

FIG. 32 shows the growth curves for the Staph aureus strain BP_001 harboring the p298 plasmid by plotting the OD600 value against time. The solid line represents average values (n=3) for uninduced cultures, and the dashed line represents the average values (n=3) for the induced cultures. The error bars represent the standard deviation of the averaged values.

Overexpression of the truncated sprA1 gene (BP_DNA_090, SEQ ID NO: 386) encoding BP_AA_014 (SEQ ID NO: 423) had an effect on the growing E. coli and Staph aureus cultures. The growth curves for the uninduced cultures began diverging from the induced cultures within 2 hrs following the addition of ATc, where the uninduced cultures continued to grow in log phase and the growth of the induced cultures slowed dramatically directly after the addition of ATc. For both strains tested, the growth rate slowed for each following time point and eventually went negative before the assay was stopped. ATc has been shown to be nontoxic and does not inhibit either species tested at the concentrations used in the experiment, so the only variable between the two cultures tested that could have caused the lower culture density in the induced cultures is the overexpressed truncated sprA1 gene.

### Example 28A. Group B Strep Kill Switch Design

The present disclosure demonstrates the insertion of an effective kill switch into the genome of Staphylococcus aureus to cause apoptosis when cultured in biological fluids such as serum, blood, plasma, and cerebrospinal fluid (CSF). These genomic switches have also been shown to be stable for over 500 generations, as provided herein, further indicating that this method of engineering cells can have many uses.

The target microorganism may be a Group B Streptococcus (Strep) species, such as Strep agalactiae, a pathogenic strain which can cause bovine mastitis and neonatal sepsis.

Hypothetical toxin/antitoxins of Strep agalactiae may be found in the genome, for example, as provided in Xie et al., 2018. Xie et al., TADB 2.0: An Updated Database of Bacterial Type II Toxin-Antitoxin Loci. Nucleic Acids Res. 2018, 46 (D1), D749-D753. https://doi.org/10.1093/nar/gkx1033. Table 53 shows a list of hypothetical Strep agalactiae toxin genes and their accession numbers. Toxin genes from other Strep species such as Strep pneumonia and Strep mutans may also be screened for potential use. Toxin genes may be PCR amplified out of the genome of Strep agalactiae using specific primer pairs. Toxin genes may also be printed out or synthesized using a DNA printing service. Toxins may be screened for lethality against Strep agalactiae by integrating the toxin gene onto a plasmid with an inducible promoter. For example, a plasmid will be used with a tet inducible promoter system, such as pRAB11, that can be induced (or derepressed) by anhydrotetracycline (ATc), a non-toxic analog of the antibiotic tetracycline. The toxin will be inserted behind the promoter on the plasmid and therefore the expression of the toxin will be induced with the addition ATc. The difference in optical density (OD) between induced and non induced strains will show the effectiveness of the toxin genes added to the plasmid. The most effective toxin genes in the inducible platform may be used to create serum inducible kill switches in Group B Strep. Table 53 shows toxin genes found using the 2.0 Toxin/Antitoxin Database. Xie et al., 2018.

TABLE 53
Potential Toxin Genes for Group B Strep

| | Hypothetical Toxins in | Strep Agalactiae |
|---|---|---|
| Accession Number | Strep agalactiae | Strain |
| WP_000384860.1 | RelE/ParE family toxin | A909 |
| WP_000700104.1 | ImmA/IrrE family toxin | A909 |
| WP_000666489.1 | RelE/ParE family toxin | A909 |
| NP_687263.1 | RelE/ParE family toxin | 2603V/R |
| AAM99341.1 | mazEF, ccd or relBE | 2603V/R |
| NP_687584.1 | Bro | 2603V/R |
| NP_688285.1 | abiGII | 2603V/R |
| NP_688826.1 | HicA | 2603V/R |
| NP_688872.1 | COG2856 | 2603V/R |
| NP_688994.1 | RelE | 2603V/R |
| NP_689104.1 | Fic | 2603V/R |

Selection of inducible promoter gene. Multiple locations in the Strep agalactiae genome may be targeted to integrate a toxin gene or genes. Promoters and genes that are upregulated in serum can be found using RNA-seq or from literature. See Table 54 for a list of Strep agalactiae genes that are necessary for growth or upregulated in serum. One site of interest could be the IgA-binding R antigen gene which is upregulated in serum. Hooven et al. The Streptococcus Agalactiae Stringent Response Enhances Virulence and Persistence in Human Blood. Infect. Immun. 2017, 86 (1). https://doi.org/10.1128/IAI.00612-17.

The toxin will be integrated behind the inducible promoter gene in such a way that it will be on the same mRNA transcript as the IgA-binding R antigen gene. The upregulated expression in serum of the IgA-binding R antigen gene will be tied or piggybacked to the toxin gene. This will increase the expression of the toxin gene in serum, creating a kill switch. Table 54 shows candidate serum inducible promoter genes in Strep agalactiae.

TABLE 54
Upregulated or Necessary Genes for
Strep agalactiae in Human Blood

| | Gene Locus | Protein | Purpose |
|---|---|---|---|
| 1 | SAK_1262 | Regulatory protein CpsA | essential for |
| | | | survival in blood |
| 2 | SAK_1255 | Capsular polysaccharide synthesis | essential for |
| | | protein CpsH | survival in blood |
| 3 | SAK_1251 | Polysaccharide biosynthesis protein | essential for |
| | | CpsL | survival in blood |
| 4 | SAK_0483 | R3H domain-containing protein | essential for |
| | | | survival in blood |
| 5 | SAK_1254 | Capsular polysaccharide | essential for |
| | | biosynthesis protein | survival in blood |
| 6 | SAK_1259 | Tyrosine-protein kinase CpsD | essential for |
| | | | survival in blood |
| 7 | SAK_1260 | Capsular polysaccharide | essential for |
| | | biosynthesis protein CpsC | survival in blood |
| 8 | SAK_1249 | UDP-N-acetylglucosamine-2- | essential for |
| | | epimerase NeuC | survival in blood |
| 9 | SAK_1900 | GTP pyrophosphokinase RelA | essential for |
| | | | survival in blood |
| 10 | SAK_1895 | PTS system transporter subunit | essential for |
| | | IIA | survival in blood |
| 11 | SAK_1258 | Glycosyl transferase CpsE | essential for |
| | | | survival in blood |
| 12 | SAK_1253 | Capsular polysaccharide | essential for |
| | | biosynthesis protein CpsJ | survival in blood |
| 13 | SAK_1248 | NeuD protein | essential for |
| | | | survival in blood |
| 14 | SAK_0186 | IgA-binding β antigen | essential for |
| | | | survival in blood |
| 15 | SAK_1256 | Polysaccharide biosynthesis | essential for |
| | | protein CpsG | survival in blood |
| 16 | SAK_1257 | Polysaccharide biosynthesis | essential for |
| | | protein CpsF | survival in blood |
| 17 | gbs0791 | Fibrinogen binding surface | invasion of |
| | | protein C FbsC | epithelial cells |

Table 54 shows genes #1-16 were found to be essential for survival in human blood based on transposon sequencing data. Hooven et al. The Streptococcus Agalactiae Stringent Response Enhances Virulence and Persistence in Human Blood. Infect. Immun. 2017, 86 (1). https://doi.org/10.1128/IAI.00612-17. Table 54 shows gene FbsC (#17) was predicted based on whole genome sequencing and characterized as a fibrinogen binding protein. Buscetta et al., 2014, FbsC, a Novel Fibrinogen-binding Protein, Promotes Streptococcus agalactiae-Host Cell Interactions http://www.jbc.org/content/289/30/21003.long. All gene candidates shown should have upregulated expression in blood or epithelial cells which makes them a good target for use in the piggyback method.

To make these insertions into the genome, a plasmid for making the genomic modifications through homologous recombination is selected. The plasmid may be pMBsacB which allows for seamless genomic knockout or integrations using a temperature selective origin of replication and a sucrose counterselection to delete the plasmid out of the genome after the homologous recombination event. Hooven et al. A Counterselectable Sucrose Sensitivity Marker Permits Efficient and Flexible Mutagenesis in Streptococcus Agalactiae. Appl. Environ. Microbiol. 2019, 85 (7). https://doi.org/10.1128/AEM.03009-18.

Homology arms and the toxin gene may be added to the pMBsacB plasmid using Gibson Assembly. Enzymatic assembly of DNA molecules up to several hundred kilobases|Nature Methods https://www.nature.com/articles/nmeth.1318/. The plasmid may be transformed into competent Strep agalactiae cells and grown at a permissive temperature to allow for replication of the plasmid. The cells will be switched to a nonpermissive temperature to force the integration of the plasmid into the genome at one of the homology arms. After confirming the integration, the plasmid may be removed from the genome, leaving the edit behind. This will be done with the addition of sucrose which acts as a counterselectant against cells that have retained the plasmid. Colonies may be screened via PCR and sequenced to ensure that the genomic edit is correct and the plasmid has been kicked out. Once the genomic edit is complete the new strain may be tested for its ability to grow in human serum by evaluating it in a serum assay as provided herein. The new kill switched strain will be inoculated into human serum and samples will be taken and plated on agar media at various time points to measure the growth of the culture by calculating colony forming units (CFU) per mL of serum. The new Strep agalactiae kill switched strain should not grow in serum but perform similar to the wild type strain in other complex media.

p296 pMBsacB_colE1. The typical protocol for using this plasmid, as stated above, requires E. coli harboring the plasmid to be grown at 30° C. or lower, which severely reduces the growth rate and extends the overall timeline for making genomic modifications in Strep by several days. In order to speed up the process of assembling plasmids to manipulate DNA in Strep, we added a derivative of the colE1 origin of replication to the pMBsacB plasmid backbone. The colE1 on comes from the plasmid pcolE1, and the modified version we used maintains a copy number around 300-500 plasmids per cell and is not temperature sensitive in E. coli. The promoter should not be recognized by Group B Strep, so it should not interfere with the temperature sensitive in vitro DNA recombination in that strain.

The DNA sequence for the colE1 on was added by linearizing the pMBsacB vector (BP_DNA_086)(SEQ ID NO: 382) by PCR amplification, and adding a PCR amplified DNA fragment containing the colE1 ori (BP_DNA_085) from the pRAB11 plasmid. The two PCR products were joined to form one circular plasmid using the Gibson Assembly kit (NEB) per the manufacturer's instructions, transformed into E. coli, and recovered and plated at 37° C. Colonies on the plates were screened for the colE1 insert, and three positive plasmids were purified and sequenced to confirm the correct DNA sequence. The new plasmid was named p296 (BP_DNA_122) and is stocked in the present inventors' plasmid database. Homology arms to target a genomic modification are added to the plasmid and its ability to recombine in the genome to make edits is tested in Group B Strep.

### Example 28B. Evolutionary Stability of Synthetic Staphylococcus Aureus Strain BP_088

In this example, the stability of a synthetic Staph aureus strain prepared according to the disclosure was evaluated over 500 generations. BP_088 (isdB::sprA1) and parent Staph aureus strain BP_001 were grown for an estimated 500 generations by passing growing cultures to fresh media for 250 hours. BP_088 performed the same in human serum prior to and after a 500 generation growth period. No mutations occurred in the DNA sequence of the integrated kill switch region during the 500 generation growth period.

Staph aureus is known to readily undergo genomic changes, and the obstacle of creating a durable genomic integration is always a concern when making edits to an organism's genome. Furthermore, demonstrating the ability to “hide” a genomic edit involving a toxin gene from the organism harboring the edit is important, especially in the Live Biotherapeutic Space (LBP). This has implications for many aspects of genetic engineering wherever there is a concern for the organism to spread once it has left the niche it was intended to inhabit.

Evolutionary stability for the piggyback genomic modification of Staph aureus synthetic strain BP_088 was tested by keeping a culture growing in exponential phase for 250 hours. Since Staph aureus has a generation time of about 30 minutes when grown in rich complex media, it was calculated that after 250 hours of growth the strain should have undergone approximately 500 generations. Maintaining a growing culture was accomplished by diluting a growing culture in a tube with fresh media every 8 to 12 hours, and then testing the strain's response to human serum both before and after the 500 generation growth period. A wild-type Staph aureus (BP_001) was grown alongside a strain containing the isdB::sprA1 integration (BP_088).

The integrations into strains BP_001 to make BP_088 and BP_121 were done using homologous recombination using the pIMAYz plasmid with plasmids p249 and p264 respectively. The edits to the genome of BP_001 to create BP_088 and BP_121 were done following the homologous recombination protocol as provided here.

Tables 55 and 56 show strains employed in the stability assay and the DNA sequence of the genomic edits made.

TABLE 55
Strains Used in Stability Assay

| | | DNA Sequence ID of |
|---|---|---|
| Strain | Genotype | genomic inserted fragment |
| BP_001 | wild type | n/a |
| BP_088 | BP_001, isdB::spra1 | BP_DNA_003 |
| BP_121 | BP_001, site2::code | BP_DNA_023 |

TABLE 56
DNA Sequences used in this Study

| Sequence | | |
|---|---|---|
| ID | Description | DNA Sequence (5′-->3′) |
| BP_DNA_ | isdB::sprA1 | CGCAGAGAGGAGGTGTATAAGGTGATGC |
| 003 | | TTATTTTCGTTCACATCATAGCACCAGT |
| | | CATCAGTGGCTGTGCCATTGCGTTTTTT |
| | | TCTTATTGGCTAAGTAGACGCAATACAA |
| | | AATAG (SEQ ID NO: 342) |
| BP_DNA_ | site2::code | CGATCTTCGACATCGGACCCTAGAACAG |
| 023 | | AACTA (SEQ ID NO: 358) |

BP_088 for 0 Generation cultures and BP_001 cultures were started in 5 mL of TSB from single colonies on a streak plate. Cultures were grown overnight in a 37° C. incubator, shaking at 240 rpm. The following morning, all cultures were diluted to 0.05 and placed in a 37° C. incubator, shaking at 240 rpm. After 2 hrs the cells were washed once with 5 ml of sterile PBS, and then were used to inoculate 5 mL of prewarmed serum and TSB to 0.05 OD 600. Immediately, the t=0 samples were taken, cultures were placed back into the incubator and serial dilutions were performed and plated. Samples were also taken at t=2, 4, 6, and 8 hrs. BP_088 500 Generation cultures and (1) BP_121 culture were started in 5 mL of TSB from single colonies on a streak plate. Cultures were grown overnight in a 37° C. incubator, shaking at 240 rpm. The following morning, all cultures were diluted to 0.05 and placed in a 37° C. incubator, shaking at 240 rpm. After 2 hrs the cells were washed once with 5 mL of sterile PBS, and then were used to inoculate 5 mL of prewarmed serum and TSB to 0.05 OD. Immediately, the t=0 samples were taken, cultures were placed back into the incubator and serial dilutions were performed and plated. Samples were also taken at t=2, 4, and 9.4 hrs.

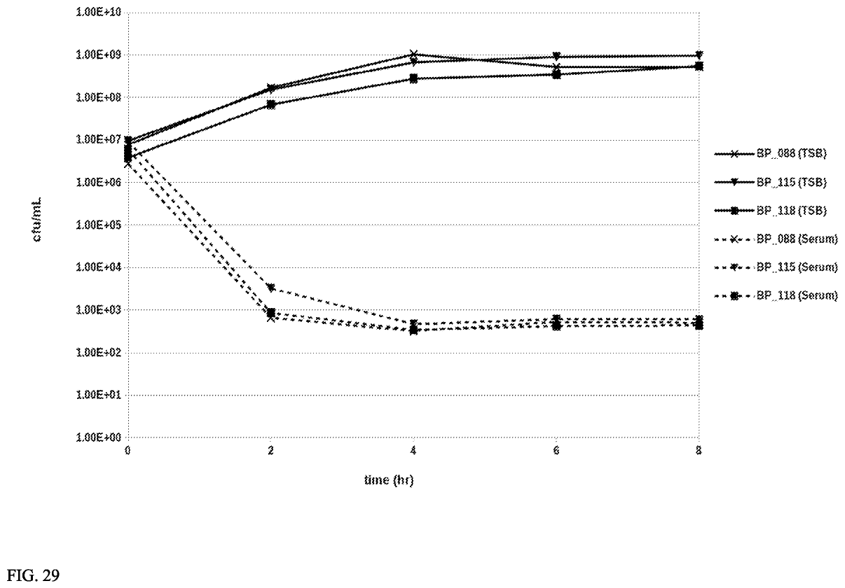

FIG. 33 shows a graph of the average (n=6) of viable CFU/mL of Staph aureus synthetic strain BP_088 (0 and 500 generation strains) when grown in human serum (dashed lines) or TSB (solid lines). BP_001 (n=6) in TSB and serum was plotted as a wild type control. Error bars represent one standard deviation of all six replicates. The BP_088-500 generation sample is represented by solid squares (▪) and the 0 generation sample (▴). Parent strain BP_001 is represented by a solid circle. Synthetic strain BP_088 exhibits functional stability over at least 500 generations as evidenced by its retained inability to grow when exposed to human serum compared to BP_088 at 0 generations. After 2 hrs in human serum, BP_088 exhibited significantly decreased cfu/mL by about 4 orders of magnitude after about 500 generations.

Sanger Sequencing. The isdB::sprA1 insert was PCR amplified from BP_088 for 0 and 500 generation strain streak plates, and sent out for sequencing. The resulting sequencing results were aligned to the BP_088 genomic map. No genetic differences, such as frameshifts or mutations, were seen in the isdB::sprA1 kill switch region. An alignment of a reference sequence for integrated sprA1 kill switch integration behind the isdB gene and the Sanger sequencing results from BP_088 at 0 and 500 generation strains. The alignment showed no mutations or changes in the synthetic strains when compared to each other or the reference sequence. Synthetic strain BP_088 exhibits genomic stability over at least 500 generations as evidenced by Sanger sequencing results. Sanger sequencing performed on the isdB::sprA1 integration region revealed there were no genetic differences between BP_088 0 and 500 generation strains in the area sequenced.

De novo sequencing of the entire genome for the BP_088 500 generation strain was also performed. (data not shown).

This example shows that the genomic integration of isdB::sprA1 into BP_001 exhibits genomic stability after roughly 500 generations.

Functional stability was also demonstrated by a serum assay that was run using the BP_088 strain that had been continuously growing for 250 hours. When the assay data is compared to the BP_088 strain that had not undergone the 250 hours of growth, they both had the same response in human serum. Both of the BP_088 strains (0 and 500 generation strains) were unable to grow in human serum in 4 hours, and the viable CFU/mL dropped by over 10⁴ from its starting concentration as shown in FIG. 33.

The stability over at least 500 generations of the inventive integrated kill switch goes far beyond previous publications that attempt to demonstrate evolutionary stability in their integrations. Stirling, Finn, et al. “Rational design of evolutionarily stable microbial kill switches.” Molecular cell 68.4 (2017): 686-697.

### Example 29. Strain Construction and Evaluation: Synthetic Microorganism Staph aureus

In this example, synthetic strain BP118 (isdB::sprA1) was constructed using target strain BP_001 having successful genomic integration of toxin gene sprA1 behind native isdB gene. BP_0118 exhibited dramatic reduction in viable cfu/mL for strain BP_118 in human serum with no difference in growth in complex media (TSB) compared to the parent strain BP_001.

The plasmid p262 was constructed and used to make this edit by transforming it into a Staph aureus strain (BP_001) and integrating it into the genome by homologous recombination. Through a double recombination process, the plasmid was fully integrated into the genome of the Staph aureus strain BP_001, then through a second homologous recombination event the plasmid is removed leaving the sprA1 gene and 5 prime untranslated region directly behind the isdB gene. The efficacy of the genomic integration was evaluated by observing its growth in human serum in vitro.

Materials and Methods

Strain Construction

The plasmid used to make the strain was plasmid p262. The DNA sequences from p262 that are integrated into the strain can be found in Table 58.

Genomic edits were made to Staph aureus using plasmid constructed from pIMAYz. Briefly, the plasmid was transformed into parent strain, grown at non-permissive temperatures for plasmid replication, screened for primary crossover strains, then grown and replated to screen colonies for the secondary crossover leaving behind the sprA1 gene. The sprA1 insertion was confirmed by Sanger sequencing of a PCR product amplified from gDNA by primers that bind to the genomic DNA outside the homology arms.

Primers used for the screening steps are found in Table 57:
i. Primary screen:
   1. DR_117, DR_533
   2. DR_117, DR_534
ii. Secondary screen:
   1. DR_534, DR_254
iii. Q5 High Fidelity PCR to confirm sprA1 integration:
   1. DR_533/DR_534
iv. Sequencing primers:
   1. DR_533, BP_949, DR_228, BP_965, BP_964, BP_950, DR_534, DR_318
v. Final confirmation:
   1. DR_534, DR_254

Following sequence confirmation of the insert, the new strain, BP_118, was stocked in 5000 glycerol and stored at −80° C.

Table 57 shows the sequences for the single stranded primers used in this study. The sequences are all in the 5 prime to 3 prime direction.

TABLE 57
Primers and Their Sequences Used to Screen and
Sequence the Insert

| Primer | |
|---|---|
| Name | Primer Sequence (5′->3′) |
| DR_117 | CCAAAGCATAATGGGATAATTAACCCTCACTAAAGGGAAC |
| | (SEQ ID NO: 544) |
| DR_254 | ATGCTTATTTTCGTTCACATCATAGCACCAGTCATCAGTG |
| | (SEQ ID NO: 545) |
| DR_533 | GATTACGCTTACATTCGCTTCTCTGTTTC (SEQ ID NO: |
| | 546) |
| DR_534 | CAGCTGTTGATAATGCCATTTTTGCACGAG (SEQ ID NO: |
| | 547) |
| BP_964 | TCAAACTTCAGCAGGTTCTAGC (SEQ ID NO: 548) |
| BP_965 | GTACCAGGTATGACTGAATGCC (SEQ ID NO: 549) |
| BP_949 | CACCTCCTCTCTGCGGATTTATTAGTTTTTACGTTTTCTAGG |
| | TAATAC (SEQ ID NO: 550) |
| DR_228 | CTATTTTGTATTGCGTCTACTTAGCCAATAAG (SEQ ID |
| | NO: 551) |
| BP_950 | AAAAACTAATAAATCCGCAGAGAGGAGGTGTATAAGGTGATG |
| | (SEQ ID NO: 552) |
| DR_318 | CGATTACTTCCCAACCATTACCTACTGTCAAC (SEQ ID |
| | NO: 553) |

Table 58 shows the DNA sequences for the homology arms and sprA1 integration. The DNA sequences used were double stranded, but the sequences shown are just one of the strands in the 5 prime to 3 prime direction. For DNA sequence BP_DNA_003, the bold sequence indicates the sprA1 reading frame, and the underlined sequence indicates the 5 prime untranslated region (control arm).

TABLE 58
DNA Fragments Used for Integration of
isdB::sprA1 (p262)

| | Seq. | |
|---|---|---|
| Name | ID | DNA Sequence (5′-->3′) |
| Upstream | BP_ | GATGAGCAAGTGAAATCAGCTATTACTGAAT |
| Homology | DNA_ | TCCAAAATGTACAACCAACAAATGAAAAAA |
| Arm | 029 | TGACTGATTTACAAGATACAAAATATGTTGT |
| | | TTATGAAAGTGTTGAGAATAACGAATCTATG |
| | | ATGGATACTTTTGTTAAACACCCTATTAAAA |
| | | CAGGTATGCTTAACGGCAAAAAATATATGGT |
| | | CATGGAAACTACTAATGACGATTACTGGAA |
| | | AGATTTCATGGTTGAAGGTCAACGTGTTAGA |
| | | ACTATAAGCAAAGATGCTAAAAATAATACT |
| | | AGAACAATTATTTTCCCATATGTTGAAGGTA |
| | | AAACTCTATATGATGCTATCGTTAAAGTTCA |
| | | CGTAAAAACGATTGATTATGATGGACAATAC |
| | | CATGTCAGAATCGTTGATAAAGAAGCATTTA |
| | | CAAAAGCCAATACCGATAAATCTAACAAAA |
| | | AAGAACAACAAGATAACTCAGCTAAGAAGG |
| | | AAGCTACTCCAGCTACGCCTAGCAAACCAAC |
| | | ACCATCACCTGTTGAAAAAGAATCACAAAA |
| | | ACAAGACAGCCAAAAAGATGACAATAAACA |
| | | ATTACCAAGTGTTGAAAAAGAAAATGACGC |
| | | ATCTAGTGAGTCAGGTAAAGACAAAACGCC |
| | | TGCTACAAAACCAACTAAAGGTGAAGTAGA |
| | | ATCAAGTAGTACAACTCCAACTAAGGTAGTA |
| | | TCTACGACTCAAAATGTTGCAAAACCAACAA |
| | | CTGCTTCATCAAAAACAACAAAAGATGTTGT |
| | | TCAAACTTCAGCAGGTTCTAGCGAAGCAAA |
| | | AGATAGTGCTCCATTACAAAAAGCAAACATT |
| | | AAAAACACAAATGATGGACACACTCAAAGC |
| | | CAAAACAATAAAAATACACAAGAAAATAAA |
| | | GCAAAATCATTACCACAAACTGGTGAAGAA |
| | | TCAAATAAAGATATGACATTACCATTAATGG |
| | | CATTACTAGCTTTAAGTAGCATCGTTGCATT |
| | | CGTATTACCTAGAAAACGTAAAAACTAATA |
| | | AATC (SEQ ID NO: 359) |
| sprA1 | BP_ | CGCAGAGAGGAGGTGTATAAGGTGATGCTT |
| Fragment | DNA_ | ATTTTCGTTCACATCATAGCACCAGTCATC |
| (insertion | 003 | AGTGGCTGTGCCATTGCGTTTTTTTCTTAT |
| sequence) | | TGGCTAAGTAGACGCAATACAAAATAG |
| | | (SEQ ID NO: 342) |
| Downstream | BP_ | GTCTTTATATTTAATTATTAAATTAACAAATT |
| Homology | DNA_ | TTAATTGGCGGATGAGGTATCCAGTTACCTC |
| Arm | 002 | GTTCGCCAATTATTTTTCGCAATATAAAAAG |
| | | TCCCACTTAAAACAATCATTTTAAGCGGGAC |
| | | TTTTTATATTGAGTAACTAAAATTATTTAGCT |
| | | GCTACTTCTTCGCCATTGTAAGAACCACAGT |
| | | TTTTACATACACGGTGTGATAATTTGTATTC |
| | | GCCACAGTTTGGGCATTCAGTCATACCTGGT |
| | | ACTGAAATTTTGAAATGCGTACGACGTTTGT |
| | | TTTTTCTAGTTTTAGAAGTTCTTCTTTTTGGT |
| | | ACTGCCATGATATATCCTCCTTAGATTATAA |
| | | ACGAAAAATACTAAATGTTAGTTTAATTAAC |
| | | AACATTATATCATTAATTAAACTACTTATTG |
| | | CTCTTTATCATATAATTGTTGTAATTTTTGAA |
| | | GCCTTGGATCAACTTGTCGTGATTCTGAATC |
| | | ATCTTGTTCTTGCTGTTTAGCAAGCTCATCTA |
| | | ATTGATCCTCATCGATTACTTCCCAACCATT |
| | | ACCTACTGTCAACATTTGGTCACTTTGCTCTG |
| | | AATAAGCTCTCATTGGTTTCTCAATAATAAC |
| | | TATATCCTCGACAATATCCTGAAGATTAACC |
| | | ATACCATCTTTAATAATGTGATAGTGTTCAT |
| | | CTACATCATCTTGATCATCGTTATACTGATTG |
| | | TACCCTTCTAAATCAAATACTTCTGTAGTAG |
| | | TTACATCTAGTGGGACTTTTACTGGTACAAG |
| | | AGTACGTGCACAAGGCATTGTATACGTTCCA |
| | | GTAATGTGAATATCCGCAACGACTTCTGTTG |
| | | ACTTAATGGTTAACTGACCTTGGATTGTAAT |
| | | TGGAGATAAATCAATTAAATCTAATGATTCT |
| | | TTTAAATTGTCAAAACTCACCGTTTGATCAA |
| | | ATTCAAATGGCTTACCTTGATATTTCCTTAAT |
| | | TGCGTAATTGAC (SEQ ID NO: 341) |

The sprA1 integration was confirmed by PCR using primers DR_534 and DR_254. BP_001 was run as a negative control to show the integration is not present. The strain was then sent for Sanger sequencing (QuintaraBio). The sequencing results showed no mutations. The data for the sequences and alignment is stored in the present inventor's Benchling account.

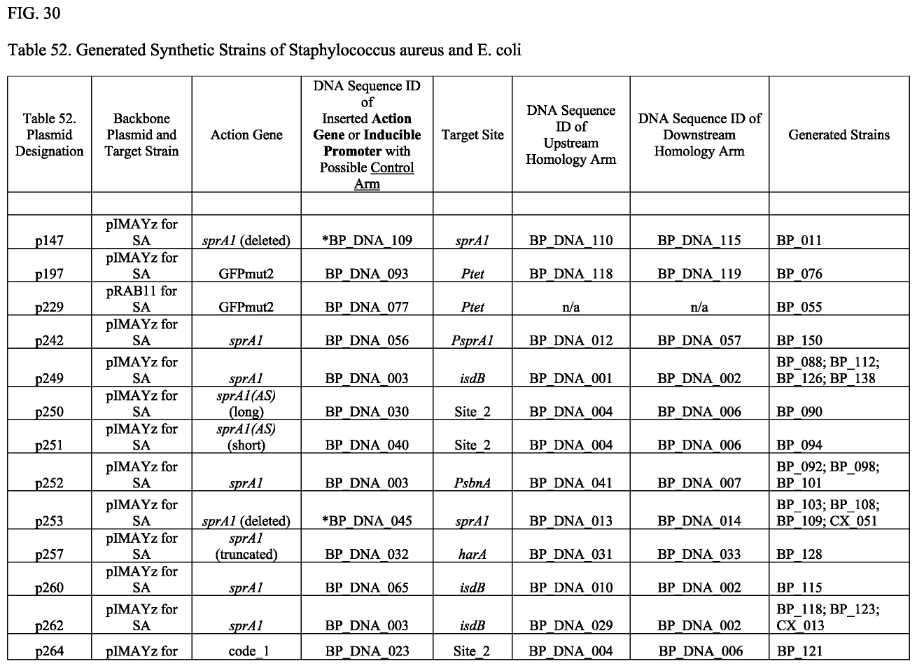

FIG. 34 shows an Agarose gel for PCR confirmation of isdb::sprA1 in BP_118. FIG. 34 shows a photograph of a 1% agarose gel that was run to check the PCR products of from the secondary recombination PCR screen with primers DR_534 and DR_254. Primer DR_534 binds to the genome outside of the homology arm, and the primer DR_254 binds to the sprA1 gene making size of the amplicon is 1367 bp for s strain with the integration and making no PCR fragment if the integration is not present. BP_001 was run as a negative control to show the integration is not present in the parent strain.

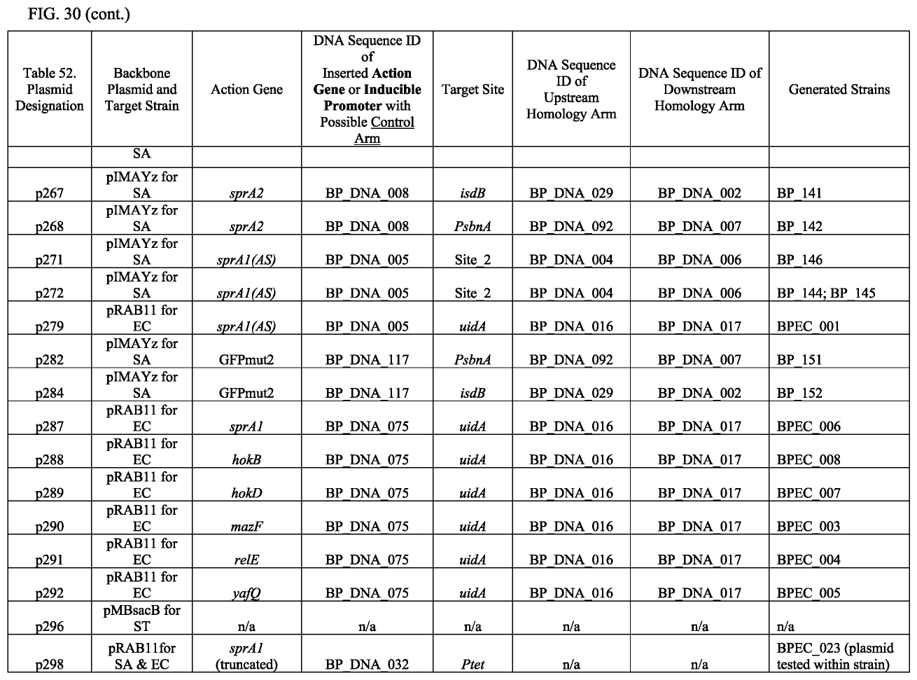

FIG. 35 shows a map of the genome of BP_118 where the sprA1 gene was inserted. It was created with the Benchling program.

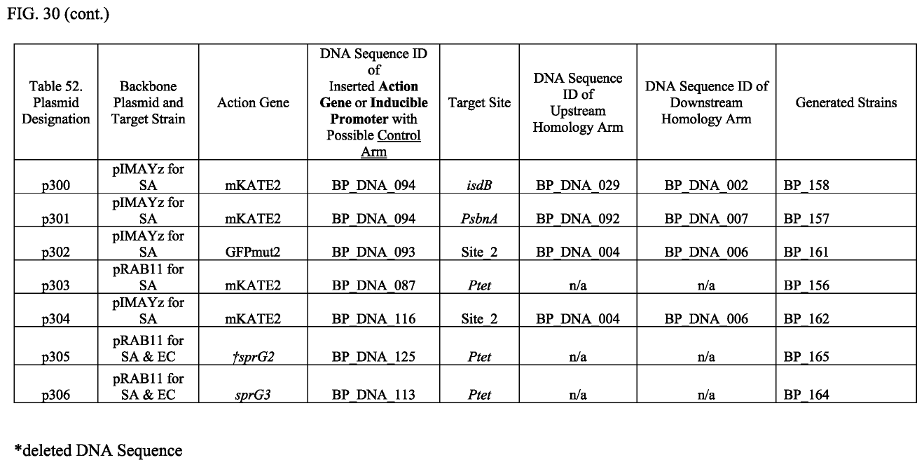

FIG. 36 shows the growth curves of strains BP_001 and BP_118 when grown in TSB media and human serum over a 4 hour period. The points plotted on the graph represent an average of 3 biological replicates and the error bars represent the standard deviation for triplicate samples. The solid lines represent the cultures grown in TSB and the dashed lines represent cultures grown in human serum. When BP_118 was evaluated in a serum assay it showed that it was able to grow similar to the wild type strain BP_001 in TSB, but unlike BP_001 cannot sustain growth in human serum.

Other Synthetic Staph aureus strains prepared in a similar fashion are shown in Table 59.

TABLE 59
Synthetic Staphylococcus aureus Strains

| Strain | Parent | Description/Genetic | | | Plasmid for |
|---|---|---|---|---|---|
| Designation | Strain | Modification | Promoter(s) | Action Gene | Integration |
| BP_001 | n/a | Wild type | n/a | n/a | n/a |
| BP_011 | BP_001 | ΔsprA1-sprA1(AS) | n/a | n/a | p147 |
| BP_055 | BP_001 | Wild type (Plasmid in | n/a | n/a | p229 |
| | | strain - p229) | | | |
| BP_076 | BP_001 | ΔsprA1::Ptet-GFP | Ptet | GFPmut2 | p197 |
| BP_088 | BP_001 | isdB::sprA1 | isdB | sprA1 | p249 |
| BP_090 | BP_011 | ΔsprA1-sprA1(AS), | gyrB | sprA1(AS)(long) | p250 |
| | | Site_2::PgyrB- | | | |
| | | sprA1(AS) (long) | | | |
| BP_092 | BP_001 | PsbnA::sprA1 | sbnA | sprA1 | p252 |
| BP_094 | BP_011 | ΔsprA1-sprA1(AS), | gyrB | sprA1(AS)(long) | p251 |
| | | Site_2::PgyrB- | | | |
| | | sprA1(AS) (short) | | | |
| BP_098 | BP_088 | isdB::sprA1, | isdB, sbnA | sprA1 | p252 |
| | | PsbnA::sprA1 | | | |
| BP_101 | BP_088 | isdB::sprA1, | isdB, sbnA | sprA1 | p252 |
| | | PsbnA::sprA1 | | | |
| BP_103 | BP_001 | ΔsprA1 | n/a | n/a | p253 |
| BP_108 | BP_098 | isdB::sprA1, | isdB, sbnA | sprA1 | p253 |
| | | PsbnA::sprA1, | | | |
| | | ΔsprA1 | | | |
| BP_109 | BP_101 | isdB::sprA1, | isdB, sbnA | sprA1 | p253 |
| | | PsbnA::sprA1, | | | |
| | | ΔsprA1 | | | |
| BP_112 | BP_090 | ΔsprA1-sprA1(AS), | gyrB, isdB | sprA1(AS)(long), | p249 |
| | | Site_2::PgyrB- | | sprA1 | |
| | | sprA1(AS)(long), | | | |
| | | isdB::sprA1 | | | |
| BP_115 | BP_001 | isdB::sprA1 (Triple | isdB | sprA1 | p260 |
| | | STOP) | | | |
| BP_118 | BP_001 | isdB::sprA1 | isdB | sprA1 | p262 |
| BP_121 | BP_001 | Site_2::code_1 | n/a | n/a | p264 |
| BP_123 | BP_103 | ΔsprA1; isdB::sprA1 | isdB | sprA1 | p262 |
| BP_126 | BP_094 | ΔsprA1-sprA1(AS), | gyrB, isdB | sprA1(AS)(short), | p249 |
| | | Site_2::PgyrB- | | sprA1 | |
| | | sprA1(AS)(short), | | | |
| | | isdB::sprA1 | | | |
| BP_128 | BP_001 | harA::sprA1* | harA | sprA1* | p257 |
| BP_138 | BP_001 | isdB::sprA1 (500 | isdB | sprA1 | p249 |
| | | generations) | | | |
| BP_141 | BP_001 | isdB::sprA2 | isdB | sprA2 | p267 |
| BP_142 | BP_001 | PsbnA::sprA2 | sbnA | sprA2 | p268 |
| BP_144 | BP_109 | isdB::sprA1, | isdB, sbnA | sprA1(AS) | p272 |
| | | PsbnA::sprA1, | | | |
| | | ΔsprA1; | | | |
| | | Site_2::PsprA1(AS)- | | | |
| | | sprA1(AS) | | | |
| BP_145 | BP_118 | isdB::sprA1; | isdB | sprA1(AS) | p272 |
| | | Site_2::PsprA1(AS)- | | | |
| | | sprA1(AS) | | | |
| BP_146 | BP_092 | PsbnA::sprA1; | sbnA | sprA1(AS) | p271 |
| | | Site 2::PsprA1(AS)- | | | |
| | | sprA1(AS) | | | |
| BP_150 | BP_001 | ΔPsprA1::PsbnA | sbnA | sprA1 | p242 |
| BP_151 | BP_001 | PsbnA::GFP | sbnA | GFPmut2 | p282 |
| BP_152 | BP_001 | isdB::GFP | isdB | GFPmut2 | p284 |
| BP_156 | BP_001 | Wild type (Plasmid in | n/a | n/a | p303 |
| | | strain - p303) | | | |
| BP_157 | BP_001 | Psbn::mKATE2 | sbnA | mKATE2 | p301 |
| BP_158 | BP_001 | isdB::mKATE2 | isdB | mKATE2 | p300 |
| BP_161 | BP_001 | Site_2::tetR_Ptet- | Ptet | GFPmut2 | p302 |
| | | GFPmut2 | | | |
| BP_162 | BP_001 | Site_2::tetR_Ptet- | Ptet | mKATE2 | p304 |
| | | mKATE2 | | | |
| CX_001 | n/a | Wild type | n/a | n/a | n/a |
| CX_013 | CX_001 | isdB::sprA1 | isdB | sprA1 | p262 |
| CX_051 | CX_013 | isdB::sprA1, ΔsprA1 | isdB | sprA1 | p253 |

*indicated truncated sprA1

Table 60 shows synthetic E. coli strains.

TABLE 60
Synthetic E. coli strains

| Strain | Parent | Description/Genetic | | | Plasmid for |
|---|---|---|---|---|---|
| Designation | Strain | Modification | Promoter(s) | Action Gene | Integration |
| BPEC_001 | IM08B | ΔuidA::PsprA1(AS)- | uidA | sprA1(AS) | p279 |
| | | sprA1(AS)_kanR | | | |
| BPEC_002 | IM08B | ΔuidA::PsprA2(AS)- | uidA | sprA2(AS) | |
| | | sprA2(AS)_kanR | | | |
| BPEC_003 | IM08B | ΔuidA::tetR_P_{XYL/tet}- | uidA | mazF | p290 |
| | | mazF_kanR | | | |
| BPEC_004 | IM08B | ΔuidA::tetR_P_{XYL/tet-} | uidA | relE | p291 |
| | | relE_kanR | | | |
| BPEC_005 | IM08B | ΔuidA::tetR_P_{XYL/tet}- | uidA | yafQ | p292 |
| | | yafQ_kanR | | | |
| BPEC_006 | IM08B | ΔuidA::tetR_P_{XYL/tet}- | uidA | sprA1 | p287 |
| | | sprA1_kanR | | | |
| BPEC_007 | IM08B | ΔuidA::tetR_P_{XYL/tet}- | uidA | hokD | p289 |
| | | hokD_kanR | | | |
| BPEC_008 | IM08B | ΔuidA::tetR_P_{XYL/tet}- | uidA | hokB | p288 |
| | | hokB_kanR | | | |
| BPEC_009 | n/a | Wild type | n/a | n/a | n/a |
| BPEC_023 | K12 | Wild type (IM08B) | n/a | n/a | n/a |
| BPEC_024 | IM08B | Wild type (Plasmid in | n/a | n/a | p306 |
| | | strain - p306 - | | | |
| | | pRAB11_Ptet-sprG3) | | | |
| BPEC_025 | IM08B | Wild type (Plasmid in | n/a | n/a | p305 |
| | | strain - p305 - | | | |
| | | pRAB11_Ptet-sprG2†) | | | |

### Example 30. Strain Construction and Evaluation: Synthetic Microorganism Staph aureus

In this example, a Staph aureus synthetic strain was constructed called BP_112 having genotype BP_001 ΔsprA1-sprA1(AS), Site_2::PgyrB-sprA1(AS)(long), isdB::sprA1. A human serum assay suggested kill switch was effective with dramatic reduction in viable CFU/mL for strain BP_112, with no difference in growth in complex media (TSB) compared to the wild-type parent strain BP_001.

BP_112 represents a kill switched strain having the expression of antisense sprA1 (sprA1(AS)) controlled by a promoter other than its native one. To make this strain, the present inventors first deleted the native sprA1 toxin gene along with the sprA1(AS) from the genome of the wild-type Staph aureus strain BP_001 using plasmid p147. Next, a PgyrB-sprA1(AS)(long) expression cassette was inserted into the non-coding region of the genome referred to as Site_2 using the plasmid p250 (Report_P018). Two versions of the sprA1(AS) were designed, the version in BP_112 represents the longer of the two versions. Finally, the isdB::sprA1 kill switch was inserted using plasmid p249. The efficacy of the genomic integration was evaluated by observing its growth in human serum in vitro.

The gyrB gene codes for the DNA gyrase subunit B and is constitutively expressed in the cell at reasonably high and stable levels. The promoter for the gene was PCR amplified from the genome of BP_001 and used to drive the expression of the antitoxin for the sprA1 gene, sprA1(AS). This was placed in the Site_2 location of the genome because we previously demonstrated that this location can be used to insert heterologous DNA without disrupting the phenotype of the cell. In order to properly test the ability of the PgyrB-sprA1(AS) cassette to sufficiently suppress the isdB::sprA1 kill switch, the native sprA1(AS) was deleted from the genome prior to making the modification into Site_2. Studies show that there is no crosstalk between the sprA toxin-antitoxin systems in a Staph cell, so by removing the sprA1(AS) the only regulation of the isdB::sprA1 kill switch will be from the PgyrB-sprA1(AS) expression cassette. Germain-Amiot et al., Nucleic acids research 47.4 (2019): 1759-1773.

Materials and Methods

Table 61 shows the three different strains that were made through multiple rounds of editing the genome to create the final strain BP_112.

TABLE 61
Strain Constructs and Parent Strains in BP_112 Lineage

| | Strain Construct | Parent | Parent Strain's |
|---|---|---|---|
| Construct | Genotype | Strain | Genotype |
| BP_011 | ΔsprA1-sprA1(AS) | BP_001 | Wild type |
| BP_090 | ΔsprA1-sprA1(AS), | BP_011 | ΔsprA1-sprA1(AS) |
| | Site_2::PgyrB- | | |
| | sprA1(AS) (long) | | |
| BP_112 | ΔsprA1-sprA1(AS), | BP_090 | ΔsprA1-sprA1(AS), |
| | Site_2::PgyrB-sprA1(AS) | | Site_2::PgyrB- |
| | (long), isdB::sprA 1 | | sprA1(AS) (long) |

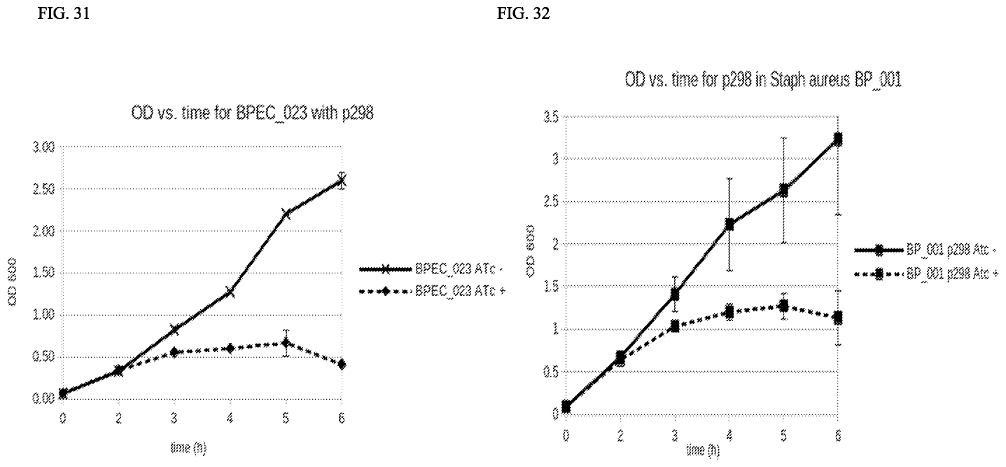

Strain Construction
1. The plasmids p147, p249, and p250 were used to make the strain over three rounds of editing the genome using the protocol outlined herein for genetic engineering of Staph aureus with pIMAYz.
   1.1. Briefly, a plasmid was transformed into parent strain, grown at non-permissive temperatures for plasmid replication, screened for primary crossover strains, then grown and replated to screen colonies for the secondary crossover leaving behind the desired insertion or deletion in the genome. The insertion/deletion was confirmed by Sanger sequencing of a PCR product amplified from gDNA by primers that bind to the genomic DNA outside the homology arms.
2. Following sequence confirmation of the insert, the new strains were stocked in 50% glycerol and stored at −80° C. to prepare strain and plasmid stock.
3. BP_112 was analyzed in an 8-hour human serum assay to assess the phenotypic response of the modified strain. BP_112 was compared to BP_001 and the serum assay was run over 8 hr. The results are included in FIG. 37.

FIG. 37 shows the average CFU/mL for BP_112 (n=3) and BP_001 (n=1) when they are grown in serum (dashed lines) and TSB (solid lines) over an 8-hour period. The error bars represent the standard deviation of the averaged values.

Three genomic modifications were made to the strain BP_001 to create the strain BP_112. First, the sprA1-sprA1(AS) genes were knocked out to remove background expression of either the sprA1 toxin or the antisense (sprA1(AS)). Next, a sprA1(AS) expression cassette was inserted into Site_2 (PgyrB-sprA1(AS)(long)). The final edit was to integrate a kill switch by inserting the sprA1 gene behind the isdB gene. All of these edits were performed successfully and have been stocked in BioPlx's database.

When evaluated in a serum assay, BP_112 (ΔsprA1-sprA1(AS), Site_2::PgyrB-sprA1(AS)(long), isdB::sprA1) was able to grow similar to the wild-type strain BP_001 in TSB, but unable to grow in human serum. This demonstrates that BP_112 successfully controlled the sprA1 kill switch using an artificial sprA1 antitoxin expression system.

### Example 31. Genomic Integration Site Selection for Optimal Expression of Action Gene: Start Site Optimization for Kill Switch

The location chosen for integrating an action gene such as a kill switch may affect the efficacy of the toxin. Gene expression can vary widely for each gene within an organism depending on the environmental conditions. As shown in this example, the efficacy of the sprA1 kill switch varies depending on the location in the genome chosen for integration.

In order to test the most optimal site for integrating an exogenous DNA sequence to create a kill switch (KS), a short growth assay was performed in pooled human serum and TSB media with the wild type Staph aureus target strain BP_001.

Briefly, overnight growth cultures of BP_001 in TSB were diluted 1:100 into fresh TSB media and grown for another 2 hours at 37° C. to sync the metabolism of the cells. Following the 2 hours growth period, the OD was taken again as the cells were washed twice and concentrated to 1 mL volumes in phosphate buffered saline (PBS). The concentrated cells were used to inoculate 3 tubes each of TSB and human serum, and grown at 37° C. in the shaking incubator for 90 minutes. Samples were taken at t=0, 30, and 90 minutes after inoculation, and the RNA was extracted and purified using the RiboPure™ RNA Purification Kit, bacteria (ThermoFisher). The RNA samples were then sent to Vertis Biotechnologie AG (Freising, Germany) for removal of the rRNA, creating a cDNA library, sequencing the cDNA library, trimming and processing the sequencing data, and mapping it to an annotated genomic sequence of a Staph aureus 502a strain. The data from the RNA seq experiment was analyzed to highlight the most differentially regulated transcripts which were then used to target the insertion of the action gene sprA1. This gene is part of a native toxin antitoxin system in BP_001 has been shown previously to be toxic when overexpressed.

Several locations in the genome were chosen to integrate the action gene in order to operably link the transcription of the gene and translation of the protein to the cell's native regulatory systems.

The genomic modifications were made using the method described in the examples above for plasmid construction using pIMAYz protocol and homologous recombination. In brief, homology arms were designed both upstream and downstream of the genomic location targeted for integration, and either a DNA fragment containing sprA1 along with a short sequence upstream of the action gene or inducible promoter was inserted into the genome. The efficacy of the integration was then determined by running growth assays in human serum or TSB.

The protocol for this example is similar to that used in the RNA-seq experiment, but after the final serum and TSB cultures were inoculated, the assay was run for 4 hours and samples were taken at t=0, 2, and 4 hours post inoculation, serially diluted by a liquid handling robot, and plated on TSB agar plates to determine the concentration of viable cells in the cultures in colony forming units per mL (CFU/mL). The growth in both TSB and pooled human serum for the engineered strains were compared to the wild type strain BP_001.

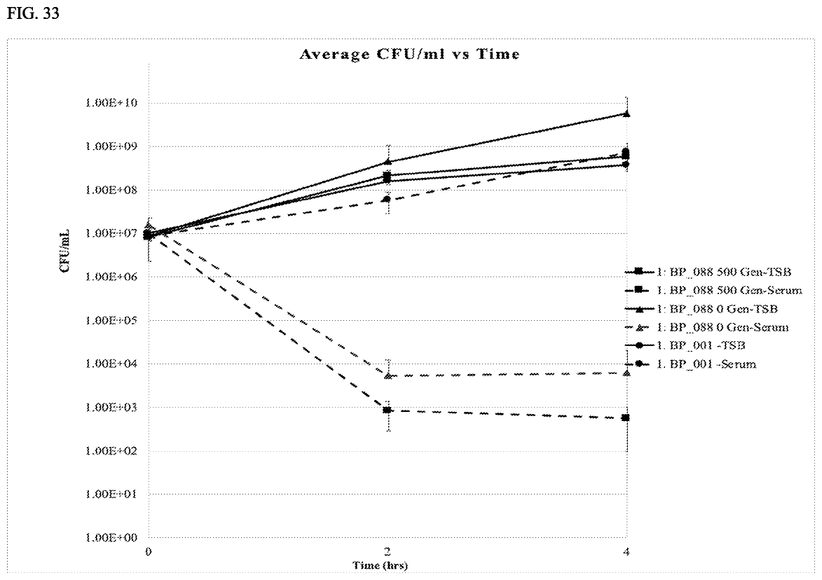

Results are shown in FIG. 38 showing the fold change in expression of 25 genes from Staph aureus at 30 and 90 minute time points in TSB and human serum. The genes shown above were most differentially regulated at the 90 minute time point between human serum and TSB broth. The number of reads for each gene was converted to transcripts per million (TPM), the replicates were averaged for each condition (n=3), normalised to the expression of the housekeeping gene gyrB, subtracted from the initial expression levels at t=0, and sorted for the most differentially expressed between the two media conditions at the 90 minute time point. The gene on the bottom of the chart (CH52_00245) had a value of 175 fold upregulation, but was cut short on this figure in order to enlarge the chart maximize the clarity of the rest of the data.

The RNA-seq results revealed many genes in BP_001 that are differentially regulated during growth in TSB and human serum. Many of the most highly differentially regulated genes between TSB and serum involve iron sequestration and acquisition from the environment. The most interesting genes for kill switch design were heavily suppressed in TSB and highly upregulated in human serum.

Table 62 shows the genes or promoters identified as good candidate locations to integrate the action gene. Genes isdB, PsbnA, and isdC are found among the top 25 genes shown in FIG. 18.

TABLE 62
Differentially Regulated Genes Identified and Targeted for Action Gene

| | Promoter or | |
|---|---|---|
| Name (Accession ID) | Gene | Description of Gene/Promoter |
| isdB (CH52_00245) | Gene | iron-regulated surface determinant protein B |
| PsbnA | Promoter | Promoter for siderophore biosynthesis proteins |
| (CH52_05140-05100) | | sbnABCDEFGHI |
| harA (CH52_10455) | Gene | Iron-regulated surface determinant protein H |
| isdC (CH52_00235) | Gene | iron-regulated surface determinant protein C |
| sbnB (CH52_05135) | Gene | 2,3-diaminopropionate biosynthesis protein SbnB |
| isdE (CH52_00225) | Gene | heme uptake system protein IsdE |

Some genes targeted for integration were not present in the top 25 differentially regulated genes, but were chosen in order to provide a spectrum of responses from the kill switch. The genes sbnB and isdE were targeted because the PsbnA promoter is a bidirectional promoter and it was hypothesized that it might be regulated in a similar manner for sbnB as it is for sbnA, and isdE is on the same operon as isdC which is among the list of top 25 genes. The harA gene was targeted due to literature claims of the protein being regulated and functionally similar to the isdB gene. Dryla et al. Journal of bacteriology vol. 189, 1 (2007): 254-64. doi:10.1128/JB.01366-06. By choosing candidate gene targets both on and off the list, a tailored spectrum of responses from the kill switch may be explored.

Table 63 shows strains that were made and tested for the sprA1 kill switch's efficacy in human serum and TSB.

TABLE 63
Strains Made to Test Location of Integration
Action Gene or Induced Promoter

| Strain Name | Genotype |
|---|---|
| BP_092 | PsbnA::sprA1 |
| BP_118 | isdB::sprA1 |
| BP_128 | harA::sprA1* |
| BP_150 | ΔPsprA1::PsbnA |

*The sprA1 gene in BP_128 was found to contain a frameshift mutation that truncates the protein by 7 amino acids, and the last 3 amino acids in the truncated protein have been changed.

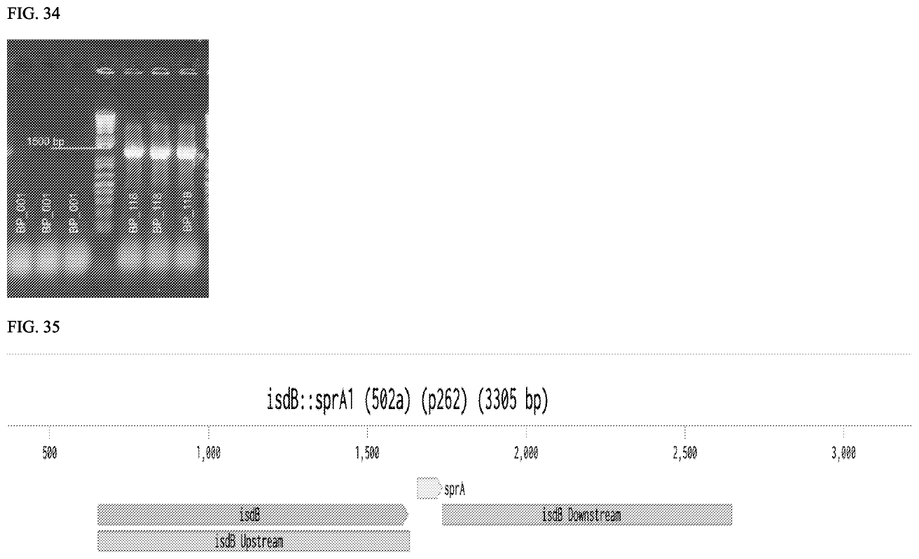

FIG. 39 shows kill switch activity as average CFU/mL of 4 Staph aureus synthetic strains with different kill switch integrations in human serum compared to parent target strain BP_001. FIG. 39 shows the viable CFU/mL of 4 different synthetic SA strains with a sprA1 kill switch integrated into 4 different locations in the genome grown in serum over 4 hours. The data is plotted as CFU/mL at three different time points and the error bars represent the standard deviation of the triplicate samples (except BP_128 which has a n=1). The CFU/mL data for all of the strains grown in TSB overlays with the BP_001 in serum on this chart and was omitted in order to produce a cleaner graph.

As shown in FIG. 39, when tested for their ability to grow in serum, strains BP_118 (isdB::spral), BP_092 (PsbnA::sprA1) and BP_128 (harA::sprA1) each exhibited a decrease in CFU/mL at both the 2 and 4 hour time points. BP_118 (isdB::spral) exhibited strongest kill switch activity as largest decrease in CFU/mL. Strain BP_150 grew only slightly slower than the wild type parent strain, but still maintained a positive growth curve during the 4 hour assay.

### Example 32. Human Plasma Kill Assay with BP_088, BP_101, BP_108, and BP_109

Several kill switched Staph aureus strains were tested for efficacy in human plasma. These same strains have been shown to quickly die in human serum, so other biological fluids are being investigated for their ability to induce the integrated kill switch (KS) and reduce the number of viable cells. Table 64 shows the strains employed in the assay.

TABLE 64
Strains Used in the Plasma KS Assay

| Strain Name | Genomic Modifications |
|---|---|
| BP_001 | Wild type Staph aureus |
| BP_088 | isdB::sprA1 |
| BP_092 | PsbnA::sprA1 |
| BP_101 | isdB::sprA1, PsbnA::sprA1 |
| BP_108 | isdB::sprA1, PsbnA::sprA1, ΔsprA1 |
| BP_109 | isdB::sprA1, PsbnA::sprA1, ΔsprA1 |

The serum assay protocol was employed as described herein except exchanging the serum growth condition with human plasma.

Human plasma is the liquid portion of blood. It is acquired by spinning to remove the cells, and still contains proteins, clotting factors, electrolytes, antibodies, antigens and hormones. Since the clotting factors are still present in the liquid, it is a difficult media to use for culturing cells. Clumps of cells and protein form over time and care was taken to homogenize the cultures before sampling. It was found that if assays longer than 3.5 hours are needed, anticoagulants should be added to the plasma prior to inoculation.

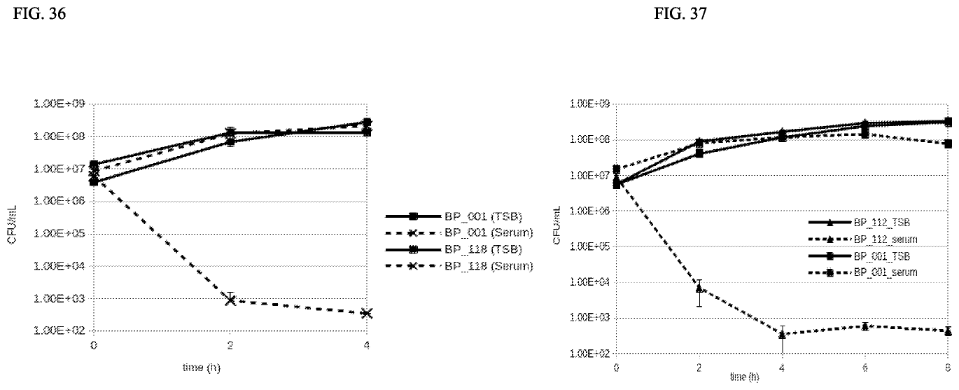

Results are shown in FIG. 40 showing a bar graph of the concentration of cfu/mL for all of the strains tested in both TSB and human plasma, at both t=0 and after 3.5 hours of growth (t=3.5). The viable cfu/mL of strains BP_088, BP_101, BP_108, and BP_109 showed over a 99% reduction after 3.5 hours in human plasma. BP_092 showed a 95% reduction in viable cfu/mL after 3.5 hours in human plasma. BP_001 showed very little difference in viable cfu/mL after 3.5 hours in human plasma. All strains grew in TSB media. The results from the assay show that the Staph aureus strains with integrated KS were unable to grow in human plasma. All of the cultures started around 1*10⁶ cfu/mL in both TSB and human serum, and after 3.5 hours of growth at 37° C. all of the TSB cultures showed an approximate 100-fold increase in cfu/mL. 502a showed a slight decrease in cfu/mL in human plasma, and the kill switched strains (BP_088, BP_092, BP_101, BP_108, BP_109) all showed a decrease in cfu/mL in plasma. The kill switched microorganisms performed well in human plasma. The results from the assay show that the Staph aureus strains with integrated KS were unable to grow in human plasma.

### Example 33. E. coli Toxin Efficacy Test

Two different E. coli strains were genomically modified under the control of the P_{XYL/Tet} promoter to incorporate putative E. coli toxins hokB, hokD, relE, mazF, and yafQ, and known S. aureus toxin sprA1. Overexpression of hokD, sprA1, and relE genes resulted in a decrease in the optical density of the synthetic E. coli cell cultures indicating they function as toxins to the host cells. In contrast, overexpression of E. coli comprising hokB, mazF, and yafQ operably linked to the inducible promoter did not demonstrate a toxic effect towards the host cells under the conditions of this assay.

Putative E. coli toxin genes were incorporated to E. coli genome and resulting strains were tested for their ability to arrest cell growth or kill living cells in a culture. A strong inducible and tightly controlled promoter system P_{XYL/Tet} was selected to perform this assay efficiently and effectively.

E. coli has many genes that have been annotated as a component of endogenous toxin-antitoxin (TA) systems. The present inventors have shown that TA systems can be exploited to develop kill switches in bacteria that are induced by environmental changes. Identifying effective toxin genes across different species and strains is a crucial part of developing such kill switches.

The RED system was used to integrate linear DNA into the genome of two different E. coli strains, a K12 background strain named IM08B (Monk et al., 2015 M Bio 6.3: e00308-15) and a strain purchased from Udder Health Systems which they use as their E. coli bovine standard. Datsenko et al., Proc. Natl. Acad. Sci. U.S.A. 97 (12), 6640-6645 (2000).

The linear DNA integrated into the genome contains a putative toxin gene behind a strong constitutive promoter P_{XYL/Tet} that contains 2 tetO sites where the tet repressor (TetR) protein tightly binds to block transcription of the putative toxin gene, as well as the tetR gene and a kanamycin resistance gene. Helle et al. “Vectors for improved Tet repressor-dependent gradual gene induction or silencing in Staphylococcus aureus.” Microbiology 157.12 (2011): 3314-3323. When anhydrotetracycline (ATc), a non-toxic form of the antibiotic tetracycline is added to the media it allosterically binds to the tetR protein changing the protein's conformation rendering it the unable to bind to the DNA at the tetO sites and block transcription of the downstream gene or genes. With the TetR proteins deactivated, the constitutive promoter is de-repressed and is uninhibited when recruiting RNA polymerase to transcribe the putative toxin gene at a high rate. The effect the toxin has on the culture can be seen by measuring the optical density (OD600) of the cultures over time. By comparing samples that have been spiked with ATc and samples that have not we can see how effective each toxin is. Top candidates will be used in the development of kill switches that are induced or repressed based on environmental conditions.

The integration of the expression cassette and kanamycin resistance gene was made by inserting it in the E. coli genome in place of the uidA gene (also called gusA) which codes for a protein called β-D-glucuronidase. The uidA gene is the first gene a three gene operon, and the integration also removes the first 4 bases in the uidB gene (also called gusB) likely disrupting or disabling the expression of it and the last gene in the operon uidC (gusC). It is nonessential for E. coli growth and its absence will not affect the efficacy of the toxins being tested here, making it a convenient location to make integrations. All of the integrations made in this report used the same homology arms for targeting the location in the genome which means that they were all made in the exact same location.

The list below shows the toxins being tested in this report and a brief description of each one:

sprA1

The sprA1 gene is native to Staph aureus, and is part of a type I toxin antitoxin system. The sprA1 gene codes for a membrane porin protein called PepA1, which accumulates in the cell's membrane and induces apoptosis in dividing cells. Schuster et al., “Toxin-antitoxin systems of Staphylococcus aureus.” Toxins 8.5 (2016): 140. The effectiveness of sprA1 in Staph aureus is provided herein and it was hypothesized it might perform similarly in E. coli. The sprA1 gene used here was PCR amplified from the genome of a 502a-like strain named i BP_001.

hokB

The hokB gene is a member of the type I toxin-antitoxin system in the hok-sok family in E. co/i. The protein has been demonstrated to insert itself into the cytoplasmic membrane and form pores that result in leakage of ATP. Wilmaerts et al. 2018. The persistence-inducing toxin HokB forms dynamic pores that cause ATP leakage. mBio 9:e00744-18. https://doi.org/10.1128/mBio 0.00744-18. Sequence analysis has shown that hokB is a homolog of the hok (host killing) gene. The hokB gene used in this report was PCR amplified from the genome of an E. coli K12 strain.

hokD

The hokD gene is a member of the type I toxin-antitoxin system in the hok-sok family in E. co/i. The stable mRNA from hokD is post transcriptionally regulated by an sRNA antitoxin sok. The hokD gene codes for a protein that has been shown to be toxic to E. co/i, resulting in loss of membrane potential, cell respiration arrest, morphological changes, and host cell death. Gerdes et al., The EMBO journal 5.8 (1986): 2023-2029. Sequence analysis has showed that hokB is a homolog of the hok (host killing) gene. The hokD gene used in this report was PCR amplified from the genome of an E. coli K12 strain.

mazF

The mazF gene is found throughout many species of bacteria, and in combination with the mazE gene, comprise a toxin antitoxin system where mazE functions as the antitoxin and mazF the toxin that has been shown to exhibit ribonuclease activity towards single or double stranded RNA resulting global translation inhibition. Aizenman et al., “An Escherichia coli chromosomal” addiction module “regulated by guanosine 3′,5′-bispyrophosphate: a model for programmed bacterial cell death.” Proceedings of the National Academy of Sciences 93.12 (1996): 6059-6063. The mazF gene used in this report was PCR amplified from the genome of an E. coli K12 strain.

relE

The relE gene is a member of the relE-relB toxin-antitoxin system in E. co/i, and has been shown to inhibit protein translation when overexpressed causing reversible cell growth. Translation inhibition occurs from relE catalyzing the cleavage of mRNA in the A site of the ribosome. Pedersen et al., “Rapid induction and reversal of a bacteriostatic condition by controlled expression of toxins and antitoxins.” Molecular microbiology 45.2 (2002): 501-510. The relE gene used in this report was PCR amplified from the genome of an E. coli K12 strain.

Methods

Table 65 shows the primer names and sequences used to construct the linear DNA fragments integrated into the genome of E. coli to test the efficacy of putative toxin genes at killing the host cells.

TABLE 65
Primers Used to Make and Sequence Integration
Fragments

| Primer | |
|---|---|
| Name | DNA Sequence (5′-->3′) |
| DR_359 | GGAACCGATTGAAGGGATTCATTTCGTTG (SEQ ID NO: |
| | 531) |
| DR_409 | CTCGGTTGCTGTGTTGCACACAGTTATCTGTGAG (SEQ ID |
| | NO: 532) |
| DR_407 | GTGTGCAACACAGCAACCGAGCGTTCTGAACAAATCCAG |
| | (SEQ ID NO: 537) |
| BM_049 | CGTACTGATTGGGTAGGTGACATATAGCCGCACCAATAAA |
| | AATTGATAATAGCTG (SEQ ID NO: 554) |
| BM_015 | GGCTATATGTCACCTACCCAATCAGTACGTTAATTTTGGC |
| | (SEQ ID NO: 555) |
| BM_014 | GGTGTATAAGGTGATGGTAAGCCGATACGTACCCGATATG |
| | (SEQ ID NO: 556) |
| BM_013 | TCGGCTTACCATCACCTTATACACCTCCTCTCTGCGG (SEQ |
| | ID NO: 557) |
| DR_634 | CAGGAGAGTTGTTGATGCATGTAACTGGGCAGTGTCTTAA |
| | AAAATCGAC (SEQ ID NO: 558) |
| DR_636 | CAGTTACATGCATCAACAACTCTCCTGGCGCACCATC (SEQ |
| | ID NO: 559) |
| DR_362 | GTTTCAGGGTTTGCAGACTGATATTCAATGACG (SEQ ID |
| | NO: 534) |
| BM_052 | GGTGTATAAGGTGATGATTCAAAGGGATATTGAATACTCG |
| | GGAC (SEQ ID NO: 560) |
| BM_027 | GCTATATGTCACTTACCCAAAGAGCGCCGCG (SEQ ID |
| | NO: 561) |
| BM_025 | CCCTTTGAATCATCACCTTATACACCTCCTCTCTG (SEQ |
| | ID NO: 562) |
| BM_024 | GCTCTTTGGGTAAGTGACATATAGCCGCACCAATAAAAATt |
| | g (SEQ ID NO: 563) |
| BM_018 | GGTGTATAAGGTGATGGCGTATTTTCTGGATTTTGACGAGC |
| | (SEQ ID NO: 564) |
| BM_019 | GGCTATATGTCACTCAGAGAATGCGTTTGACCGCCTCG |
| | (SEQ ID NO: 565) |
| BM_017 | AAAATACGCCATCACCTTATACACCTCCTCTCTGCGG (SEQ |
| | ID NO: 566) |
| BM_016 | CGCATTCTCTGAGTGACATATAGCCGCACCAATAAAAATT |
| | G (SEQ ID NO: 567) |
| DR_244 | CATCACCTTATACACCTCCTCTCTGCGG (SEQ ID NO: |
| | 568) |
| DR_661 | CTGAGGAGTAAGTGACATATAGCCGCACCAATAAAAATTG |
| | ATAATAGCTG (SEQ ID NO: 569) |
| DR_659 | CGCAGAGAGGAGGTGTATAAGGTGATGAAGCAGCAAAAG |
| | GCGATGTTAATCG (SEQ ID NO: 570) |
| DR_660 | GTGCGGCTATATGTCACTTACTCCTCAGGTTCGTAAGCTGT |
| | GAAGAC (SEQ ID NO: 571) |
| DR_674 | GTCCAGGTAAGTACCCAGGAAACAGCTATGACCATG (SEQ |
| | ID NO: 572) |
| DR_673 | AGCTGTTTCCTGGGTACTTACCTGGACGTGCAGGCCATG |
| | (SEQ ID NO: 573) |
| DR_672 | GGAGGTGTATAAGGTGATGAAGCACAACCCTCTGGTGGTG |
| | (SEQ ID NO: 574) |
| DR_675 | GGTTGTGCTTCATCACCTTATACACCTCCTCTCTGCGG |
| | (SEQ ID NO: 575) |
| DR_280 | GTAGACGCAATACAAAATAGGTGACATATAGCCGCACC |
| | (SEQ ID NO: 576) |
| DR_278 | CGCAGAGAGGAGGTGTATAAGGTGATGCTTATTTTCGTTCA |
| | CATC (SEQ ID NO: 577) |
| DR_228 | CTATTTTGTATTGCGTCTACTTAGCCAATAAG (SEQ ID |
| | NO: 578) |

DNA Fragment Construction

The list below shows the primer pairs (and templates) used to PCR amplify the fragments that were assembled to construct the DNA fragments integrated into the genome of E. coli.
1) ΔuidA::tetR_P_{XYL/Tet}-sprA1_kanR
   a) Upstream HA—DR_359/DR_409 (E. coli gDNA)
   b) kanR—DR_407/DR_637 (pCasSA plasmid)
   c) tetR_P_{XYL/tet}—DR_634/DR_280 (pRAB11 plasmid)
   d) sprA1—DR_278/DR_228 (Staph aureus gDNA)
   e) Downstream HA—DR_362/DR_636 (E. coli gDNA, K12)
2) ΔuidA::tetR_P_{XYL/Tet}-hokB_kanR
   a) Upstream HA—DR_359/DR_409 (E. coli gDNA)
   b) kanR—DR_407/DR_674 (pCasSA plasmid)
   c) tetR_P_{XYL/tet}—DR_634/DR_675 (pRAB11 plasmid)
   d) hokB—DR_672/DR_673 (E. coli gDNA, K12)
   e) Downstream HA—DR_362/DR_636 (E. coli gDNA, K12)
3) ΔuidA::tetR_P_{XYL/Tet}-hokD_kanR
   a) Upstream HA—DR_359/DR_409 (E. coli gDNA)
   b) kanR—DR_407/DR_661 (pCasSA plasmid)
   c) tetR_P_{XYL/tet}—DR_634/DR_244 (pRAB11 plasmid)
   d) hokD—DR_659/DR_660 (E. coli gDNA, K12)
   e) Downstream HA—DR_362/DR_636 (E. coli gDNA, K12)
4) ΔuidA::tetR_P_{XYL/Tet}-relE_kanR
   a) Upstream HA—DR_359/DR_409 (E. coli gDNA, K12)
   b) kanR—DR_407/BM_016 (pCasSA plasmid)
   c) tetR_P_{XYL/tet}—BM_017/DR_634 (pRAB11 plasmid)
   d) relE—BM_018/BM_019 (E. coli gDNA, K12)
   e) Downstream HA—DR_362/DR_636 (E. coli gDNA, K12)
5) ΔuidA::tetR_P_{XYL/tet}-yafQ_kanR
   a) Upstream HA—DR_359/DR_409 (E. coli gDNA, K12)
   b) kanR—BM_024/DR_407 (pCasSA plasmid)
   c) tetR_P_{XYL/tet}—BM_025/DR_634 (pRAB11 plasmid)
   d) yafQ—BM_052/BM_027 (E. coli gDNA, K12)
   e) Downstream HA—DR_362/DR_636 (E. coli gDNA, K12)
6) ΔuidA::tetR_P_{XYL/Tet}-mazF_kanR
   a) Upstream HA—DR_359/DR_409 (E. coli gDNA, K12)
   b) kanR—BM_049/DR_407 (pCasSA plasmid)
   c) tetR_P_{XYL/tet}—BM_013/DR_634 (pRAB11 plasmid)
   d) mazF—BM_015/BM_014 (E. coli gDNA)
   e) Downstream HA—DR_362/DR_636 (E. coli gDNA)

All of the fragments listed above were PCR amplified using Q5 Hot Start DNA polymerase (NEB) per the manufacturer's instructions and run on a 1-2% agarose gel to confirm good amplification from the template DNA. The PCR fragments were then purified using a PCR cleanup kit (Qiagen) and assembled by the stitch PCR protocol outlined in Report_SOP036. The primer pair DR_362/DR_359 was used to create the single linear DNA fragment used to make each integration. This PCR product incorporates the 5 fragments used in the stitch PCR (Upstream HA, kanR, tetR_P_{XYL/tet}, putative toxin gene, Downstream HA).

Table 66 shows the DNA sequences for the putative toxin genes tested and described in this report.

TABLE 66
DNA Sequences of the Toxins Tested in Efficacy
Test

| | DNA | |
|---|---|---|
| Toxin | Sequence | |
| Name | ID | DNA Sequence (5′-->3′) |
| sprA1 | BP_DNA_ | ATGCTTATTTTCGTTCACATCATAGCACCAGTCA |
| | 035 | TCAGTGGCTGTGCCATTGCGTTTTTTTCTTATTG |
| | | GCTAAGTAGACGCAATACAAAATAG (SEQ ID |
| | | NO: 364) |
| hokB | BP_DNA_ | ATGAAGCACAACCCTCTGGTGGTGTGTCTGCTC |
| | 067 | ATTATCTGCATTACGATTCTGACATTCACACTCC |
| | | TGACCCGACAAACGCTCTACGAACTGCGGTTCC |
| | | GGGACGGTGATAAGGAGGTTGCTGCGCTCATGG |
| | | CCTGCACGTCCAGGTA (SEQ ID NO: 374) |
| hokD | BP_DNA_ | ATGAAGCAGCAAAAGGCGATGTTAATCGCCCTG |
| | 068 | ATCGTCATCTGTTTAACCGTCATAGTGACGGCAC |
| | | TGGTAACGAGGAAAGACCTCTGCGAGGTACGAA |
| | | TCCGAACCGGCCAGACGGAGGTCGCTGTCTTCA |
| | | CAGCTTACGAACCTGAGGAGTAA (SEQ ID NO: |
| | | 375) |
| mazF | BP_DNA_ | ATGGTAAGCCGATACGTACCCGATATGGGCGAT |
| | 069 | CTGATTTGGGTTGATTTTGACCCGACAAAAGGT |
| | | AGCGAGCAAGCTGGACATCGTCCAGCTGTTGTC |
| | | CTGAGTCCTTTCATGTACAACAACAAAACAGGT |
| | | ATGTGTCTGTGTGTTCCTTGTACAACGCAATCAA |
| | | AAGGATATCCGTTCGAAGTTGTTTTATCCGGTCA |
| | | GGAACGTGATGGCGTAGCGTTAGCTGATCAGGT |
| | | AAAAAGTATCGCCTGGCGGGCAAGAGGAGCAA |
| | | CGAAGAAAGGAACAGTTGCCCCAGAGGAATTAC |
| | | AACTCATTAAAGCCAAAATTAACGTACTGATTG |
| | | GGTAG (SEQ ID NO: 376) |
| yafQ | BP_DNA_ | ATGATTCAAAGGGATATTGAATACTCGGGACAA |
| | 070 | TATTCAAAGGATGTAAAACTTGCACAAAAGCGT |
| | | CATAAGGATATGAATAAATTGAAATATCTTATG |
| | | ACGCTTCTTATCAATAATACTTTACCGCTTCCAG |
| | | CTGTTTATAAAGACCACCCGCTGCAAGGTTCAT |
| | | GGAAAGGTTATCGCGATGCTCATGTCGAACCGG |
| | | ACTGGATCCTGATTTACAAACTTACCGATAAACT |
| | | TTTACGATTTGAGAGAACTGGAACTCACGCGGC |
| | | GCTCTTTGGGTAA (SEQ ID NO: 377) |
| relE | BP_DNA_ | ATGGCGTATTTTCTGGATTTTGACGAGCGGGCAC |
| | 071 | TAAAGGAATGGCGAAAGCTGGGCTCGACGGTAC |
| | | GTGAACAGTTGAAAAAGAAGCTGGTTGAAGTAC |
| | | TTGAGTCACCCCGGATTGAAGCAAACAAGCTCC |
| | | GTGGTATGCCTGATTGTTACAAGATTAAGCTCCG |
| | | GTCTTCAGGCTATCGCCTTGTATACCAGGTTATA |
| | | GACGAGAAAGTTGTCGTTTTCGTGATTTCTGTTG |
| | | GGAAAAGAGAACGCTCGGAAGTATATAGCGAG |
| | | GCGGTCAAACGCATTCTCTGA (SEQ ID NO: |
| | | 378) |

Table 67 shows one strand of the double stranded DNA sequences that were used as homology arms to target the location of the integrations described in this report. For sequence BP_DNA_075 (SEQ TD NO: 379), the underlined sequence is the P_{XYL/tet} promoter sequence and the bold portion is the sequence for the tetR gene. The bold portion in BP_DNA_076 (SEQ ID NO: 380) corresponds to the kanR gene.

TABLE 67
DNA Sequences and Sequence IDs for ΔuidA Homology
Arms

| | DNA | |
|---|---|---|
| DNA Name | Sequence ID | DNA Sequence (5′-->3′) |
| Upstream HA | BP_DNA_ | GGAACCGATTGAAGGGATTCATTTCGTTGACTA |
| | 016 | TATGGTCGAGTCCATTGTCTCTCTCACCCATGAA |
| | | GCCTTTGGACAACGGGCGCTGGTGGTTGAAATT |
| | | ATGGCGGAAGGGATGCGTAACCCACAGGTCGC |
| | | CGCCATGCTTAAAAATAAGCATATGACGATCAC |
| | | GGAATTTGTTGCCCAGCGGATGCGTGATGCCCA |
| | | GCAAAAAGGCGAGATAAGCCCAGACATCAACA |
| | | CGGCAATGACTTCACGTTTACTGCTGGATCTGA |
| | | CCTACGGTGTACTGGCCGATATCGAAGCGGAAG |
| | | ACCTGGCGCGTGAAGCGTCGTTTGCTCAGGGAT |
| | | TACGCGCGATGATTGGCGGTATCTTAACCGCAT |
| | | CCTGATTCTCTCTCTTTTCGGCGGGCTGGTGATA |
| | | ACTGTGCCCGCGTTTCATATCGTAATTTCTCTGT |
| | | GCAAAAATTATCCTTCCCGGCTTCGGAGAATTC |
| | | CCCCCAAAATATTCACTGTAGCCATATGTCATG |
| | | AGAGTTTATCGTTCCCAATACGCTCGAACGAAC |
| | | GTTCGGTTGCTTATTTTATGGCTTCTGTCAACGC |
| | | TGTTTTAAAGATTAATGCGATCTATATCACGCTG |
| | | TGGGTATTGCAGTTTTTGGTTTTTTGATCGCGGT |
| | | GTCAGTTCTTTTTATTTCCATTTCTCTTCCATGGG |
| | | TTTCTCACAGATAACTGTGTGCAACACAG (SEQ |
| | | ID NO: 352) |
| Downstream | BP_DNA_ | GTTTCAGGGTTTGCAGACTGATATTCAATGACG |
| HA | 017 | GCTGCGCAACGATACGTACCACATTCTCACGCG |
| | | TCGATTTGAAGCAGATGAAGTAAAGCACCATTC |
| | | CGGCAATCGCCAGCACAATTGTCCAGAAATGGT |
| | | ATACCGACACCATCTCTTCCGGGCTGGAGTTCTT |
| | | AATGCTCGGTCCTATCAGAAATGCCAGGCAGAC |
| | | AAAGGTCAATGAAGCGGCAATCCCACGAGCCG |
| | | CGCCCAGACGGGCGCGGGATTGTGGTTGTTGGG |
| | | TCATCGCGGTAGCAAGTGAACCATAAGGAATAT |
| | | TCACCAGGCTGTAGCAAAGCCCGAGGCCCATGT |
| | | AGGTCAAATATGCATACACCACTTTGCTACCAT |
| | | GGCTCCAGTCGGTCAGCACCCAGAATACCAGCA |
| | | CGCTGAAGATCATTAACGGCGCAGTACCGAAGA |
| | | GTAAAAACGGGCGGAATTTTCCCCAGCGGGTAT |
| | | TCACACTGTCCACCACTCGTCCGGCAAAGACGT |
| | | CGGCGAAGGCATCGAATACCCGCACCAGTAAC |
| | | AGCATGGTGCCCGCCGCAGCGGCACCGACGCCA |
| | | GCGACGTCGGTGTAGTAACTCAACAGGAAGAG |
| | | CGCCCCCATTGCGAAGGCGAAGTTATTGGCGAC |
| | | GTCACCGAGGCTGTAGCCGACGATGGTGCGCCA |
| | | GGAGAGTTGTTGAT (SEQ ID NO: 353) |
| tetR_P_{WYL-tet} | BP_DNA_ | GCATGTAACTGGGCAGTGTCTTAAAAAATCGAC |
| | 075 | ACTGAATTTGCTCAAATTTTTGTTTGTAGAATTA |
| | | GAATATATTTATTTGGCTCATATTTGCTTTTTAA |
| | | AAGCTTGCATGCCTGCAGGTCGACGGTATCGAT |
| | | AACTCGACATCTTGGTTACCGTGAAGTTACCAT |
| | | CACGGAAAAAGGTTATGCTGCTTTTAAGACCC |
| | | ACTTTCACATTTAAGTTGTTTTTCTAATCCGC |
| | | ATATGATCAATTCAAGGCCGAATAAGAAGGC |
| | | TGGCTCTGCACCTTGGTGATCAAATAATTCG |
| | | ATAGCTTGTCGTAATAATGGCGGCATACTAT |
| | | CAGTAGTAGGTGTTTCCCTTTCTTCTTTAGCG |
| | | ACTTGATGCTCTTGATCTTCCAATACGCAACC |
| | | TAAAGTAAAATGCCCCACAGCGCTGAGTGCA |
| | | TATAATGCATTCTCTAGTGAAAAACCTTGTTG |
| | | GCATAAAAAGGCTAATTGATTTTCGAGAGTT |
| | | TCATACTGTTTTTCTGTAGGCCGTGTACCTAA |
| | | ATGTACTTTTGCTCCATCGCGATGACTTAGTA |
| | | AAGCACATCTAAAACTTTTAGCGTTATTACGT |
| | | AAAAAATCTTGCCAGCTTTCCCCTTCTAAAGG |
| | | GCAAAAGTGAGTATGGTGCCTATCTAACATC |
| | | TCAATGGCTAAGGCGTCGAGCAAAGCCCGCT |
| | | TATTTTTTACATGCCAATACAATGTAGGCTGC |
| | | TCTACACCTAGCTTCTGGGCGAGTTTACGGG |
| | | TTGTTAAACCTTCGATTCCGACCTCATTAAGC |
| | | AGCTCTAATGCGCTGTTAATCACTTTACTTTT |
| | | ATCTAATCTAGACATCATTAATTCCTCCTTTTT |
| | | GTTGACATTATATCATTGATAGAGTTATTTGTCA |
| | | AACTAGTTTTTTATTTGGATCCCCTCGAGTTCAT |
| | | GAAAAACTAAAAAAAATATTGACACTCTATCAT |
| | | TGATAGAGTATAATTAAAATAAGCTCTCTATCA |
| | | TTGATAGAGTATGATGGTACCGTTAACAGATCT |
| | | GAGCCGCAGAGAGGAGGTGTATAAGGTG (SEQ |
| | | ID NO: 379) |
| kanR | BP_DNA_ | GTACCCAGGAAACAGCTATGACCATGTAATACG |
| Fragment | 076 | ACTCACTATACGGGGATATCGTCGGAATTGCCA |
| | | GCTGGGGCGCCCTCTGGTAAGGTTGGGAAGCCC |
| | | TGCAAAGTAAACTGGATGGCTTTCTTGCCGCCA |
| | | AGGATCTGATGGCGCAGGGGATCAAGATCTGAT |
| | | CAAGAGACAGGATGAGGATCGTTTCGCATGAT |
| | | TGAACAAGATGGATTGCACGCAGGTTCTCCG |
| | | GCCGCTTGGGTGGAGAGGCTATTCGGCTATG |
| | | ACTGGGCACAACAGACAATCGGCTGCTCTGA |
| | | TGCCGCCGTGTTCCGGCTGTCAGCGCAGGGG |
| | | CGCCCGGTTCTTTTTGTCAAGACCGACCTGT |
| | | CCGGTGCCCTGAATGAACTGCAGGACGAGGC |
| | | AGCGCGGCTATCGTGGCTGGCCACGACGGG |
| | | CGTTCCTTGCGCAGCTGTGCTCGACGTTGTC |
| | | ACTGAAGCGGGAAGGGACTGGCTGCTATTGG |
| | | GCGAAGTGCCGGGGCAGGATCTCCTGTCATC |
| | | TCACCTTGCTCCTGCCGAGAAAGTATCCATC |
| | | ATGGCTGATGCAATGCGGCGGCTGCATACGC |
| | | TTGATCCGGCTACCTGCCCATTCGACCACCA |
| | | AGCGAAACATCGCATCGAGCGAGCACGTACT |
| | | CGGATGGAAGCCGGTCTTGTCGATCAGGATG |
| | | ATCTGGACGAAGAGCATCAGGGGCTCGCGCC |
| | | AGCCGAACTGTTCGCCAGGCTCAAGGCGCGC |
| | | ATGCCCGACGGCGAGGATCTCGTCGTGACCC |
| | | ATGGCGATGCCTGCTTGCCGAATATCATGGT |
| | | GGAAAATGGCCGCTTTTCTGGATTCATCGAC |
| | | TGTGGCCGGCTGGGTGTGGCGGACCGCTATC |
| | | AGGACATAGCGTTGGCTACCCGTGATATTGC |
| | | TGAAGAGCTTGGCGGCGAATGGGCTGACCGC |
| | | TTCCTCGTGCTTTACGGTATCGCCGCTCCCG |
| | | ATTCGCAGCGCATCGCCTTCTATCGCCTTCTT |
| | | GACGAGTTCTTCTGAGCGGGACTCTGGGGTTC |
| | | GAGAGCTCGCTTGGACTCCTGTTGATAGATCCA |
| | | GTAATGACCTCAGAACTCCATCTGGATTTGTTC |
| | | AGAACGCTCGGTTG (SEQ ID NO: 380) |

The DNA fragments were integrate into the genome of E. coli using the plasmid pKD46 which contains the RED genes to help facilitate recombination of the transformed DNA and the genome. The protocol for making edits using this method is as follows:
1) Make electrocompetent E. coli cells per the protocol outlined in Report_SOP030 and use plasmid pKD46 to transform the fresh electrocompetent cells.
   a) Recover at 30° C. for 1 hour and plate the cells on LB agar plates with carbenicillin (100 μg/mL) and incubate at 30° C. for 36-48 hours.
2) When colonies are visible, using a sterile inoculation loop, pick a single colony and restreak for single colony isolation on a fresh LB agar plate with carbenicillin (100 μg/mL) and incubate the plates at 30° C. for 36-48 hours.
3) When single colonies have grown to sufficient size, prepare E. coli pKD46 electrocompetent cells again per the protocol outlined in Report_SOP030 with the following modifications:
   a) Add carbenicillin to all growth media prior to transformation to a working concentration of 100 μg/mL.
   b) Culture cells at 30° C. for overnight growth (Day 1 Step 1.4).
   c) At Day 2 Step 7, after 2 hours of growth at 30° C., add 3.5 mL of 10% arabinose to the cell culture, transfer the flask to the 37° C. shaking incubator at 250 rpm, and incubate the culture for another 45 minutes to 1 h.
   d) Follow the remaining steps for preparing the cells for transformation while paying extra attention to keeping the cells cold but not frozen at all times.
   e) Use >400 ng of linear DNA to transform the E. coli cells and recover at 37° C. for 3 hours.
   f) Plate various volumes (25, 100, 250 μL) of recovered cells on LB agar plates with 50 μg/mL kanamycin added and incubate the plates overnight (16-24 hours) at 37° C.
4) The following day the cells were screened by colony PCR using a primer that binds outside the homology arms and one primer that binds to the putative toxin gene behind the P_{XYL/Tet} promoter.
   a) PCR products were run on a 1% agarose gel to check for colonies that are positive for the integration.
   b) Colonies that were positive for the integration had the DNA insertion and the surrounding region sequenced to confirm that there were no mutations in the inserted fragments.
   c) Once the sequence was confirmed it was struck out for single colony isolation and used in growth assays to observe the effects of inducing and overexpressing the putative toxin genes.

Results:

All of the toxins described above were successfully integrated into the genome an E. coli strain, along with the tetR and kanR genes described previously. Sequencing results showed no mutations in the DNA inserted into the genomes or the surrounding area (˜1000 bases upstream or downstream of the integration site. The synthetic strains are shown in Table 68.

TABLE 68
List of E. coli Synthetic Strains

| Strain Name | Genotype |
|---|---|
| BPEC_003 | (K12) ΔuidA::tetR_P_{XYL/tet}-mazF_kanR |
| BPEC_004 | (K12) ΔuidA::tetR_P_{XYL/tet}-relE_kanR |
| BPEC_005 | (K12) ΔuidA::tetR_P_{XYL/tet}-yafQ_kanR |
| BPEC_006 | (K12) ΔuidA::tetR_P_{XYL/tet}-sprA1_kanR |
| BPEC_007 | (K12) ΔuidA::tetR_P_{XYL/tet}-hokD_kanR |
| BPEC_008 | (K12) ΔuidA::tetR_P_{XYL/tet}-hokB_kanR |

Growth Assays for the newly constructed E. coli synthetic strains shown in Table 28 were performed as follows.
1. Start one 5 mL LB+kanamycin (50 μg/mL) culture for each toxin/strain to be tested from a single colony on fresh agar plates. Incubate overnight (12-18 h) in the shaking incubator at 37° C.
2. The next day measure OD600 of overnight cultures.
3. Calculate the volume (V) of overnight (O-N) culture needed to inoculate a fresh 5 mL of LB media to an OD600 of 0.05, V=(0.05/O-N OD600)×5000 μL.
4. Inoculate 2 tubes of LB+kanamycin (50 μg/mL) for each strain being tested using the calculated volume of inoculum from Step 3.
5. Immediately after inoculation and before putting the tubes in the 37° C. shaking incubator, briefly vortex to mix the culture and take the OD for the initial OD reading (t=0). Do not dilute because the OD will be very low (should be around 0.05).
6. Put culture tubes in the shaking incubator at 37° C. for 1 hour.
7. After 1 hour measure and record the OD600 readings, then add 4 μL of anhydrotetracycline (ATc) (1 mg/mL stock solution) to one set of the culture tubes (this is referred to as the spiked samples).
8. Place cultures back in the 37° C. shaking incubator and measure and record the OD600 values every hour for 4 more hours.
9. Enter recorded ODs in a table and plot the data on a graph to show the growth curves for all of the strains tested. The data below was collected from multiple days of experiments.

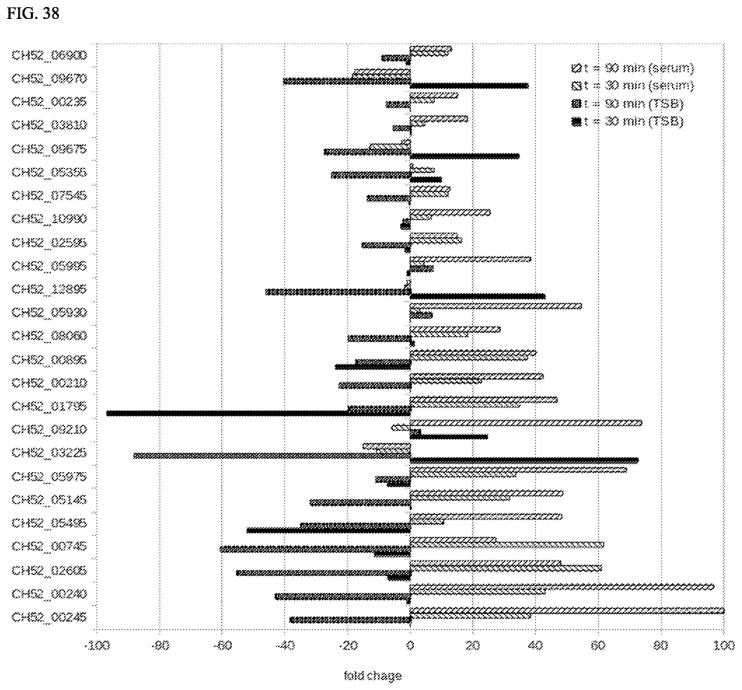

Results are shown in FIG. 41 to 44.

FIG. 41 shows a graph of the growth curves of (4) different E. coli (sprA1) strains grown in LB with an inducible sprA1 gene integrated in the genome. The dashed line represents the cultures that were induced with ATc and the solid line represents cultures that did not get induced with ATc. All 4 strains that got ATc spiked in the media at 1 h showed a significant decrease in the culture density throughout the entire assay compared to the cultures that did not get an ATc spike. Two different types of target E. coli strains were employed: strains 1, 2, and 15 are from E. coli K12-type target strain IM08B, and strain 16 is the bovine E. coli target strain obtained from Udder Health Systems. All induced strains showed significant decrease in growth over 2-5 hr time points.

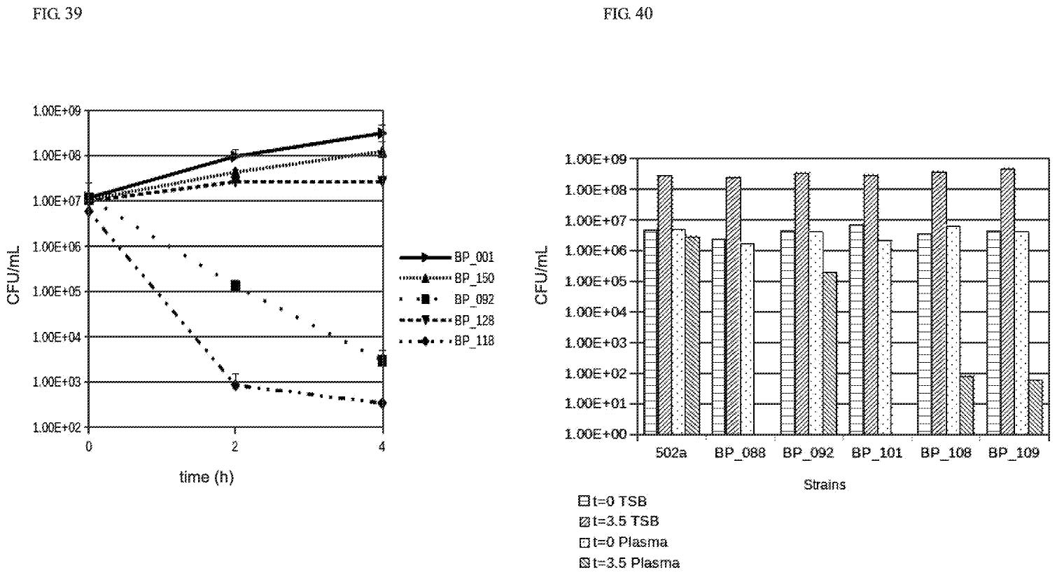

FIG. 42 shows a graph of the growth curves as OD600 values over 5 hrs with of (4) different synthetic E. coli isolates grown in LB with an inducible hokB or hokD gene integrated in the genome of K12-type E. coli target strain IM08B. Samples were induced by adding ATc to the culture 1 h post inoculation. The dashed line represents the cultures that were spiked with ATc to induce expression of the putative toxin genes and the solid line represents cultures that did not get induced by ATc. The hokD sample exhibited a diverging curve between the induced and uninduced samples. The hokB_1 is the bovine E. coli strain from Udder Health Systems and the spiked and unspiked samples grew much faster than the other 3 strains tested here

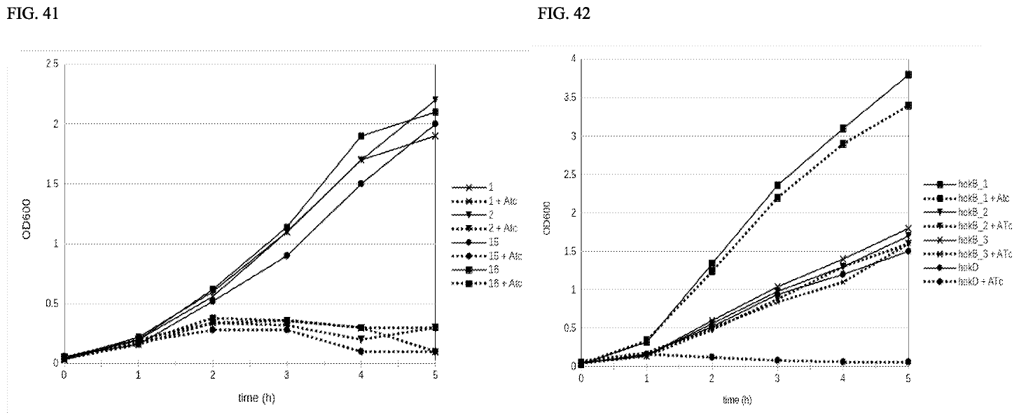

FIG. 43 shows a graph of the average (n=3) growth curves as OD600 values over 5 hrs of two synthetic E. coli strains with relE or yafQ gene integrated in the genome (n=3) grown in LB (+/−ATc). The dashed lines represent the cultures that were spiked with ATc to induce expression of the putative toxin genes and the solid lines represent cultures that did not get induced by ATc. The error bars represent one standard deviation for the averaged OD600 values for each strain. The relE gene showed diverging curves between the cultures that were induced and the uninduced cultures, where the induced cultures had significantly lower OD600 readings. The induced yafQ cultures showed a slightly slower growth between hours 2 and 4 than the uninduced cultures, but at 5 hours the two groups had nearly identical OD600 values.

Neither synthetic E. coli having genomically integrated mazF gene nor wild type bovine E. coli strain (Udder Health Systems) exhibited statistically significant growth curves over 5 hrs when grown in LB with and without the addition of ATc at t=1 hr to the culture (data not shown).

Synthetic E. coli having genomically integrated sprA1, hokD, and relE genes operably linked to inducible gene when overexpressed exhibited significantly reduced growth in liquid culture. Both sprA1 and hokD showed a fast kill switch activity on the density of the cultures, while relE seemed to have a toxic effect on the host cells 2 hours post induction of the gene.

Two different E. coli target strains were genomically modified under the control of the ATc-inducible P_{XYL/Tet} promoter to incorporate putative E. coli toxins hokB, hokD, relE, mazF, and yafQ, and known S. aureus toxin sprA1. Overexpression of hokD, sprA1, and relE genes resulted in a decrease in the optical density of the synthetic E. coli cell cultures indicating they function as toxins to the host cells. In contrast, overexpression of E. coli comprising hokB, mazF, and yafQ operably linked to the inducible promoter did not demonstrate a toxic effect towards the host cells under the conditions of this assay.

### Example 34. Kill Switch in Synovial Fluid

This example evaluated the phenotypic responses of two synthetic S aureus BP_109 (kill switch) and BP_121 (control) in human synovial fluid (SF).

Synovial fluid is a viscous liquid found in articulating joints. The two principal functions of synovial fluid are to provide lubrication within articulating joint capsules, and to act as a nutrient transport medium for surrounding tissues. Nutrients are transported to synovial joints via the blood plasma, and likewise waste products are carried away from synovial fluid via the bloodstream. Like plasma, synovial fluid is a serum-derived fluid. Synovial fluid is essentially begins as ultra-filtered blood plasma. As such, many synovial fluid components are derived from blood plasma, and the proteome compositions of the two fluids have been shown to be highly comparable.

Septic arthritis is a condition caused by bacterial infection of joint tissue. Various microorganisms can cause septic arthritis and Staphylococcus aureus is a leading cause of the condition. Septic arthritis can originate from the spread of bacteria from another infection locus in the body via the bloodstream, or from direct inoculation of the joint via puncture wounds or surgery.

Based on the shared origin and compositional similarities among serum, plasma and synovial fluid, it was predicted that the synthetic microorganisms comprising a kill switch would be effective in synovial fluid and reduce cell viability. Two strains were selected for the assay, BP_109 and BP_121. BP_109 is a modified kill switch strain, while BP_121 is phenotypically wild type S. aureus that served as the control group. Control BP_121 (site 2::code 1) has only a small integration in a non-coding region used for identification by PCR only. Table 69 shows genotypes and sequences of genomically inserted DNA fragments of synthetic S. aureus strains used in this assay.

TABLE 69
Synthetic S. aureus Strains Used synovial fluid assay

| | | DNA Sequence ID of |
|---|---|---|
| Strain | Genotype | Genomic Inserted Fragment |
| BP_121 | BP_001, site2::code 1 | BP_DNA_023 |
| BP_109 | BP_001, isdB::sprA1, | BP_DNA_003 |
| | PsbnA::sprA1, | BP_DNA_040 |
| | ΔsprA1 | BP_DNA_045 |

Media use in the synovial fluid assay are shown in table 70.

TABLE 70
Media and Other Solutions ued in synovial fluid assay

| Name | Description | Manufacturer | Part Number |
|---|---|---|---|
| TSB | Tryptic Soy Broth (minus | Teknova | T1395 |
| | glucose) | | |
| SF | Human Synovial Fluid | BioChemed | BC51519HSF |
| | (Pooled, Mixed Gender) | | |
| PBS | Phosphate Buffered Saline | Teknova | P0200 |
| TSA | Tryptic Soy Agar Culture | Teknova | T0144 |
| Plates | Plates | | |

Table 71 shows DNA Sequences employed in synthetic strains. All DNA insertions and deletions are double stranded DNA. Only single stranded sequences are listed above.

TABLE 71
DNA Sequences used in BP_109 and BP_121

| Sequence | | Sequence |
|---|---|---|
| ID | Genotype | of Insert or Deletion |
| BP_DNA_ | BP_001, | Cgatcttcgacatcggaccctagaac |
| 023 | site2::code | agaacta (SEQ ID NO: 358) |
| BP_DNA_ | isdB::sprA1 | CGCAGAGAGGAGGTGTATAAGGTGAT |
| 003 | | GCTTATTTTCGTTCACATCATAGCAC |
| | | CAGTCATCAGTGGCTGTGCCATTGC |
| | | GTTTTTTTCTTATTGGCTAAGTAGAC |
| | | GCAATACAAAATAG (SEQ ID NO: |
| | | 342) |
| BP_DNA_ | PsbnA::sprA1 | CGCAGAGAGGAGGTGTATAAGGTGAT |
| 040 | | GCTTATTTTCGTTCACATCATAGCAC |
| | | CAGTCATCAGTGGCTGTGCCATTGC |
| | | GTTTTTTTCTTATTGGCTAAGTAGAC |
| | | GCAATACAAAATAG (SEQ ID NO: |
| | | 365) |
| BP_DNA_ | ΔsprA1 | ATATAATAGTAGAGTCGCCTATCTCTC |
| 045 | (deletion of | AGGCGTCAATTTAGACGCAGAGAGGA |
| | 5′ end) | GGTGTATAAGGTGATGCTTATTTTCGT |
| | | CTACATCATAGCAC (SEQ ID NO: |
| | | 368) |

Synovial Fluid Assay protocol involves culture preparation, serial dilutions, plating and colony counting as shown below.
1. Culture Preparation
   1.1. Cultures were started by inoculating 5 mL TSB with single colonies of BP_109 and BP_121 in 14 mL sterile culture tubes, and placing them in the shaking incubator at 37° C. and 240 rpm to grow overnight. (3 tubes each for biological replicates)
   1.2. The following morning, the overnight cultures were cut back to 0.05 OD600 in 5.5 mL of fresh TSB.
      1.2.1. OD600 was measured in 1 cm cuvette on NanoDrop spectrophotometer.
      1.2.2. The resulting OD600 values were used to calculate the volume of overnight culture needed to inoculate fresh TSB to 0.05 OD600.
      1.2.3. Fresh 5.5 mL TSB cultures were inoculated with appropriate volumes of overnight culture and incubated for 2 hrs (37° C., 240 rpm) in order to get the cells growing in log phase again.
   1.2.4. After the 2 hour incubation the OD600 was measured for each culture.
   1.2.5. The cultures were then washed in sterile PBS.
      1.2.5.1. Cultures were centrifuged to pellet the cells using the swing out rotor (3500 rpm, 5 mins, RT), and washed with 5 mL PBS.
      1.2.5.2. Cultures were centrifuged to pellet the cells again, and resuspended in 1 mL sterile PBS.
   1.2.6. The OD600 values obtained after the 2 hour incubation were used to calculate the volume needed to inoculate 1.8 mL of Synovial Fluid or TSB to 0.05 OD600.
      1.2.6.1. (Measured OD600)(X mL)=(0.05 OD600)(1.8 mL)
   1.2.7. The following cultures were then inoculated in pre-warmed 37° C.:
      1.2.7.1. BP_109 in TSB (1 tube)
      1.2.7.2. BP_109 in Synovial Fluid (3 tubes)
      1.2.7.3. BP_121 in TSB (1 tube)
      1.2.7.4. BP_121 in Synovial Fluid (3 tubes)
   1.2.8. After addition of inoculum, cultures were mixed by pulse vortex and 100 uL samples were taken for determining cfu/mL by dilution plating (see below).
   1.2.9. The cultures were immediately placed in the 37° C. shaking incubator (240 rpm) and samples were taken after 2 hrs and again at 4 hrs to determine cfu/mL by dilution plating.
2. Serial Dilutions and Culture Plating
   2.1. Dilution plating was performed using the Opentrons OT-2 robot following the protocol described in Report_SOP017.
      2.1.1. Dilutions were carried out to a concentration where 30-300 colonies grew from plating 100 μL of diluted sample on TSA plates.
3. Incubation and Colony Counting
   3.1. TSA plates were incubated overnight for 12-16 hrs at 37° C.
   3.2. The following morning, plates were removed from the incubator and colony counting was performed to determine the concentration of viable cells at each time point (cfu/mL).
      3.2.1. Multiple dilutions were plated in duplicate for each condition at each time point, only plates with 30-300 colonies were used to calculate cfu/mL values.

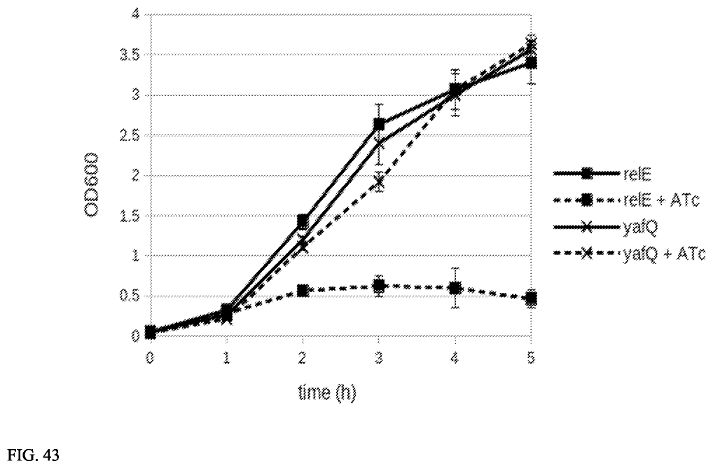

Results for the synovial fluid assay are shown in FIG. 44 showing a graph the concentrations of synthetic S. aureus BP_109 and BP_121 cells grown in in TSB and human synovial fluid over the course of a 4 hour growth assay. Both BP_121 (control) and BP_109 (kill switch) cultures grew in TSB. BP_109 showed a rapid decrease in viable cfu/mL in the synovial fluid condition.

The present study demonstrated that BP_109 behaves similarly in human synovial fluid as it does in human plasma and human serum. BP_109 in SF showed significant decreases in viable cfu/mL over the first two hours of the assay, and by the hour 4 only a few viable colonies remained. In contrast, BP_121 grew in synovial fluid at a rate similar to the BP_121 and BP_109 TSB control groups. The results of this assay support the conclusion that the genetically engineered kill switch strain BP_109 functions as designed. The kill switch appears to be activated in human synovial fluid which severely and suddenly reduces the concentration of viable cells in the fluid.

### Example 35. Kill Switch in Cerebrospinal Fluid

This experiment evaluated the phenotypic responses of synthetic S. aureus strains BP_109 (kill switch) and BP_121 (control) in rabbit cerebrospinal fluid (CSF) enriched with 2.5% human serum. BP_109 performed similarly in serum enriched CSF as it does in human plasma, human serum, and human synovial fluid. BP_109 in serum enriched CSF showed significant decreases in cfu/mL over the course of 6 hours.

Cerebrospinal fluid is a clear liquid that surrounds the central nervous system (CNS). CSF principally functions as a mechanical barrier to cushion the CNS, and is involved in the auto-regulation of cerebral blood flow. Additionally, CSF functions as a transport media, providing nutrients from the bloodstream to surrounding tissues and removing wastes, and as such has often been referred to as a “nourishing liquor.” Despite this characteristic as a nutrient transport media, CSF is a nutrient poor environment compared to blood plasma. Numerous species of bacteria, including S. aureus, have been reported to exhibit little to no growth in CSF in vitro. This phenomenon might be an evolutionary means to protect the central nervous system from bacterial invaders via nutrient sequestration. Additionally, CSF is protected from microbial invasion by the meninges, which are membranes that surround the brain and spinal cord. CSF occupies the subarachnoid space between the two innermost meninges, arachnoid mater and pia mater. Bacterial infection of these tissues produces inflammation, referred to as meningitis Aguilar et. al. “Staphylococcus aureus Meningitis Case Series and Literature Review.” Medicine, vol. 89, no. 2, pp. 117-125, 2010

There are two scenarios in which S. aureus meningitis may be likely to arise. The first is postoperative meningitis. This occurs when the structural integrity of the of the meningeal linings encompassing CSF become compromised during surgical procedures. In these circumstances infections can occur when bacteria are able to enter during surgery, spread from a nearby contagious infection, or enter through CSF shunts. The second pathogenic mechanism for S. aureus meningitis is known as hematogenous meningitis, which is a secondary infection caused by bacteremic spread from an infection outside of the CNS. In cases of methicillin resistant Staphylococcus aureus (MRSA) meningitis, the vast majority have been reported to be nosocomial in origin. Pinado et al. “Methicillin-Resistant Staphylococcus aureus Meningitis in Adults.” Medicine, vol. 91, no. 1, pp. 10-17, 2011.

Given the relative inability of S. aureus to grow in healthy spinal fluid in vitro, it was deemed appropriate to create conditions to mimic potentially susceptible states in vivo. The present study investigated the efficacy of a synthetic Staph aureus having a kill switch in CSF under mock conditions of a perturbed state, where the usually highly protected cerebrospinal fluid environment has become contaminated with nutrient rich serum, thus creating an environment susceptible to infection. Rabbit CSF was spiked with 2.5% human serum. It was hypothesized that the addition of this low level of serum would stimulate enough metabolic activity for kill switch activation in BP_109, resulting in dramatic reduction in viability. BP_121 (control), and synthetic strain BP_109 comprising a kill switch genomic modification, as described in example 15 were subjected to the CSF assay.

The protocol for the CSF assay was similar to that described in example 15, except synovial fluid was replaced with contaminated CSF which was rabbit CSF (New Zealand White RabbitRabbit Cerebrospinal Fluid, BioChemed) spiked with 2.5% human serum.

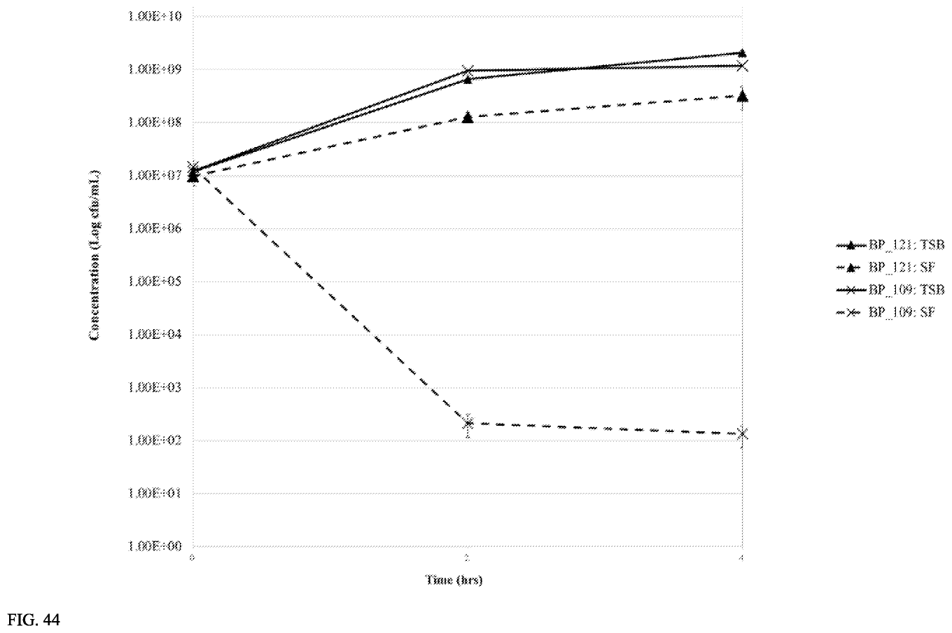

FIG. 45 shows a graph of the concentration of viable BP_109 and BP_121 cells in TSB and Serum Enriched CSF over the course of a 6 hour assay. Both BP_121 (control) and BP_109 (kill switch) cultures grew in TSB. BP_121 also grew in CSF enriched with 2.5% human serum; however, BP_109 showed a rapid decrease in cfu/mL in the CSF condition.

This experiment evaluated the phenotypic responses of BP_109 and BP_121 in cerebrospinal fluid. Both strains are genetically engineered versions of S. aureus 502a, however, BP_121 has only a small integration in a non-coding region, and is phenotypically wild type. BP_109 is a genetically engineered kill switch strain of 502a (BP_001) which has previously been shown to significantly decrease in cfu/mL after being introduced to human serum, plasma, and synovial fluid.

Despite the fact that S. aureus is capable of causing life-threatening meningitis, previous studies have shown that does not readily grow, or die, but rather remains stable in CSF in vitro. As such, human serum (2.5%) was added to CSF in order to provide basic nutrients necessary for growth. Under these serum enriched CSF conditions BP_109 decreased in viability by several orders of magnitude. The results of this assay support the conclusion that the genetically engineered kill switch strain BP_109 functions as designed in contaminated CSF. The kill switch appears to be activated in 2.5% serum enriched rabbit CSF and BP_109 dies.

### Example 36. Bacteremia Study in Vivo Staphylococcus aureus

An in vivo bacteremia mouse study to compare the clinical effects (bacteremia) in mice subjected to a tail vein injection of two Staph aureus microorganisms modified with kill switch (KS) technology with wild-type (WT) Staphylococcus aureus (SA).

In this study, all mice injected with 10\hat{ }7 CFU/mouse of synthetic Staph aureus (KS) survived the entire 8 day duration of the study and demonstrated health, lack of clinical symptoms, and maintained body weight. All positive controls (mice injected with 10\hat{ }7 CFU/mouse of WT SA) died or were determined moribund and euthanized by ethical standards.

Normal weight was defined as weight within 15% of the initial weight.

Synthetic strains of Staph aureus comprising kill switch genomic modifications exhibited good efficacy in human plasma, human serum, human synovial fluid, and contaminated rabbit cerebrospinal fluid assays in vitro as described herein. The present Bacteremia Study was designed to test the efficacy of two KS modified Staph strains, BP_109 and CX_013 (Table 32), in the prevention of bacteremia after tail vein injection. BP_001 and CX_001, are wild type organisms of the same lineage as BP_109 and CX_013, respectively, and were included in the study as positive controls.

Based on the kill switch activity of synthetic KS strains in vitro, it was hypothesized that the kill switch would also perform as designed in vivo and initiate artificially programmed cell death upon entering the bloodstream. It was predicted that mice in the kill switch groups would remain healthy and fail to develop bacteremic infections, and that wild type groups would develop severe bacteremia, or be diagnosed as moribund and euthanized. Results of the study met these expectations.

Materials

BioPlx engineered two organisms for use in the mouse bacteremia study. The two synthetic Staph aureus organisms are designated BP_109 and CX_013 and were generated through the genomic alteration of organisms BP_001 and CX_001, respectively as shown in Table 72.

TABLE 72
Strains Used in Mouse Bacteremia Study

| Strain | Genotype |
|---|---|
| BP_001 | wild type |
| BP_109 | BP_001, isdB::sprA1, PsbnA::sprA1, ΔsprA1 |
| CX_001 | wild type isolated from microbiome swab |
| CX_013 | isdB::sprA1 |

Table 73 shows the strains used and the targeted concentration of cells in CFU/mouse.

TABLE 73
Groups, Treatment and Dosing

| | Treatment | | |
|---|---|---|---|
| | (100 uL tail vein | Target Dose | |
| Group | injection) | (CFU/mouse) | Designation |
| 1 | Vehicle (Sterile PBS) | NA | Negative Control |
| 2 | Killed BP_001 | 10\hat{ }7 | Negative Control - |
| | | | Wild Type |
| 3 | BP_001 | 10\hat{ }7 | Positive Control - |
| | | | Wild Type |
| 4 | BP_109 | 10\hat{ }7 | Test Group - Kill |
| | | | Switch |
| 5 | CX_001 | 10\hat{ }7 | Positive Control - |
| | | | Wild Type |
| 6 | CX_013 | 10\hat{ }7 | Test Group - Kill |
| | | | Switch |

Methods

Test Article Preparation

The test articles were prepared as follows. Briefly, single colonies of each strain were picked and grown overnight in liquid tryptic soy broth (TSB). For each strain, 1 mL of the overnight culture was used to inoculate 100 mL of fresh TSB and then incubated for another 14 hours. After the 14 hour incubation period, the cells were washed three times with phosphate buffered saline (PBS), a sample was serially diluted and plated on tryptic soy agar (TSA) plates to determine the CFU/mL, and the cells were stored overnight at 4° C.

The next day the CFU plates were counted and the actual concentration was determined. Using the calculated CFU/mL cell concentrations of the PBS cell solutions, final test articles were prepared at the appropriate concentrations. An aliquot of BP_001 was made and treated with 70% isopropyl alcohol to kill the cells, then washed three times with PBS to remove any alcohol. While the alcohol treatment group was incubating, the remaining treatment groups were prepared from the PBS cell solutions. The test articles were then hand delivered to the facility where the dosing and observations occurred.

Non-GLP Mouse Study

A non-GLP exploratory study was performed. Five BALB/c male mice were assigned to each group for experimentation. Each animal was dosed once intravenously on study Day 0 by tail vein injection using sterile PBS as the vehicle. The treatment and dosing by group is shown in (Table 33).

BALB/c mice were selected as a suitable model for a bacteremia study as well as intravenous injection according to literature reports. Stortz et al. “Murine models of sepsis and trauma: can we bridge the gap?.” ILAR journal 58.1 (2017): 90-105. The bacteria levels (10\hat{ }7 CFU/mouse) were chosen based on similar peer-reviewed articles studying bacteremia effects in mice of the same species and of similar age. van den Berg et al. “Mild Staphylococcus aureus skin infection improves the course of subsequent Endogenous S. aureus bacteremia in mice.” PloS one 10.6 (2015): e0129150. Prior to injection, the animals were allowed 48 hours to acclimate to the new environment and body weights were obtained and recorded on study Day 0. Body weights were measured once each morning for the duration of the study. Mortality and morbidity checks were performed twice a day (once in the morning and once in the evening) for the duration of the study. Animals who experienced a 20% or greater loss in weight were deemed suitable for euthanasia.

All procedures conformed to USDA guidelines for animal care and handling. Study design and animal usage were approved by the USDA certified (84-R-0081) and OLAW assured facility (A4678-01) performing the study.

Results

The pre-dose body weights ranged from 21.9 to 30.7 g. Clinical observations and body weight measurements were all normal for Groups 1, 2, 4 and 6 (negative controls and kill switch test groups) with the exception of one observation of hypoactivity in one mouse from Group 4 on study Day 2.

Numerous abnormal clinical observations, including (but not limited to) significant weight loss, rough coat, milky eye excretions and death, were observed for all mice in Groups 3 and 5 (positive controls). All animals from Group 3 (BP_001 subjects) were deceased upon conclusion of the study. Three of the five animals from Group 5 (CX_001 subjects) were deceased upon conclusion of the study and the two survivors had beyond 20% weight loss declaring both fit for euthanasia.

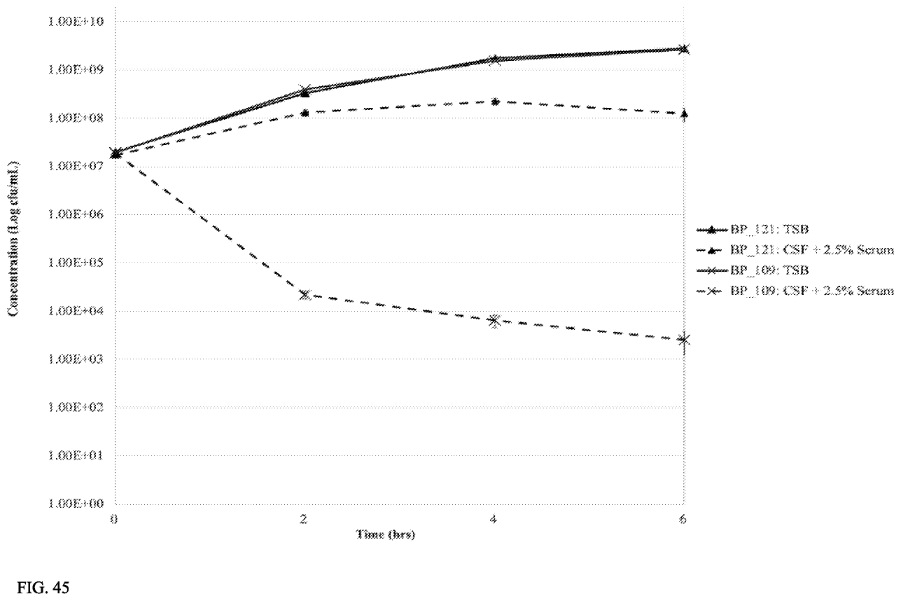

Bacteremia results are depicted in FIG. 46. The graph values were generated by averaging and normalizing the body weight for each group of interest. Normalization was performed by dividing the group (average) weight at each time point by the initial group (average) weight. Each time point average was generated using only surviving mice. A graphic is shown at the bottom of the graph to represent adverse clinical observations and mortality.

A Bacteremia Study was performed in vivo in mice to compare the clinical effects (bacteremia) in a mouse model following tail vein injection of 10\hat{ }7 Staphylococcus aureus (SA) modified with kill switch (KS) technology or wild type (WT) target strains. The organisms modified with KS technology were designed to initiate artificially programmed cell death upon interacting with blood, serum, or plasma of the mammalian host.

All mice injected intravenously via tail vein injection with KS organisms as well as negative controls were healthy with no adverse clinical symptoms for the duration of the study, excluding one observation of hypoactivity which subsided by next observation. All mice injected with WT organisms experienced a wide variety of abnormal clinical observations, significant morbidity, and were either deceased or were fit for euthanasia by ethical standards. This study demonstrated the efficacy and safety of the kill switch KS technology with 100% survival and health of all test subjects over the 8 days of study. Synthetic Staph aureus strains comprising a kill switch may significantly de-risk protective organisms for use in methods for prevention and treatment of infectious disease.

### Example 37. SSTI Study in Vivo Staphylococcus aureus

An in vivo study was performed to compare the clinical effects in an SSTI (skin and soft tissue infection) model in mice subjected to subcutaneous injections with wild-type (WT) Staphylococcus aureus (SA) vs two SA organisms modified with kill switch (KS) technology. Study duration was ten days.

In this study, all mice injected with 10\hat{ }7 synthetic Staphylococcus aureus KS strains demonstrated health in both clinical symptoms (i.e. no abscess formation) and maintained body weight for the duration of the study, while half of the positive controls (mice injected with WT SA strains) developed abscesses.

An in vivo mouse Skin and Soft Tissue (SSTI) Study was designed to test the efficacy of two KS-modified SA strains, BP_109 and CX_013 (Table 34), in the prevention of SSTI after subcutaneous injection. BP_001 and CX_001, are wild-type (WT) organisms of the same lineage as BP_109 and CX_013, respectively, and were included in the study as positive controls. Based on the kill switch efficacy achieved in vitro and in an in vivo Bacteremia Study it was hypothesized that the KS would also perform as designed in vivo after subcutaneous injection and initiate artificially-programmed cell death upon entering the body under the skin. It was predicted that mice in the KS groups would remain healthy throughout the study and fail to develop SSTI infections. The WT groups were expected to develop abscess formation (indicative of SSTI).

Materials

The SSTI study employed two synthetic Staph aureus KS strains designated BP_109 and CX_013 and two WT target microorganisms BP_001 and CX_001 as shown in Table 74.

TABLE 74
Staphylococcus aureus trains used in SSTI Study

| | | DNA Sequence ID of |
|---|---|---|
| Strain | Genotype | genomic inserted fragment |
| BP_001 | wild type | n/a |
| BP_109 | BP_001, isdB::sprA1, | BP_DNA_003 |
| | PsbnA::sprA1, | BP_DNA_003 |
| | ΔsprA1 | BP_DNA_045 |
| CX_001 | wild type | n/a |
| CX_013 | CX_001, isdB::sprA1 | BP_DNA_003 |

Table 75 shows treatment groups, target dose and strain types employed in the SSTI study.

TABLE 75
SSTI Treatment Groups, Treatment and Dosing

| | | | Actual Dose | |
|---|---|---|---|---|
| | Treatment | Target Dose | Administered | Strain |
| Group | (100 uL SC) | (CFU/mouse) | (CFU/mouse) | Type |
| 1 | Vehicle (Sterile | n/a | n/a | n/a |
| | PBS) | | | |
| 2 | Killed BP_001 | 10\hat{ }7 | 0 | WT (neg) |
| 3 | BP_001 | 10\hat{ }7 | 6.00E+06 | WT (pos) |
| 4 | BP_109 | 10\hat{ }7 | 1.61E+07 | KS (test) |
| 5 | CX_001 | 10\hat{ }7 | 1.21E+07 | WT (pos) |
| 6 | CX_013 | 10\hat{ }7 | 7.95E+06 | KS (test) |

SC—Subcutaneous Injection;
Neg—Negative;
Pos—Positive;
WT—Wild Type;
KS—Kill Switch

SC—Subcutaneous Injection; Neg—Negative; Pos—Positive; WT—Wil Type; KS—Kill Switch

Test Article Preparation

The test articles were prepared according to a protocol described by Malachowa et al. 2013. Malachowa, Natalia, et al. “Mouse model of Staphylococcus aureus skin infection.” Mouse Models of Innate Immunity. Humana Press, Totowa, N.J., 2013. 109-116.

Briefly, single colonies of each strain were picked and grown overnight in liquid tryptic soy broth (TSB). For each strain, 1 mL of the overnight culture was used to inoculate 100 mL of fresh TSB and then incubated for another 14 hours. After the 14-hour incubation period, the cells were washed three times with phosphate buffered saline (PBS), a sample was serially diluted and plated on tryptic soy agar (TSA) plates to determine the CFU/mL, and the cells were stored overnight at 4° C. The next day the CFU plates were counted and the actual concentration was determined. Using the calculated CFU/mL cell concentrations of the PBS cell solutions, final test articles were prepared at the appropriate concentrations. One aliquot of BP_001 was made and treated with 70% isopropyl alcohol to kill the cells, then washed three times with PBS to remove any alcohol. While the alcohol treatment group was incubating, the remaining treatment groups were prepared from the PBS cell solutions. The test articles were then hand-delivered to the facility where the dosing and observations occurred.

A non-GLP exploratory study was performed over 10 days. Five BALB/c male mice (Charles River) were assigned to each group for experimentation. Each animal was dosed once subcutaneously on study Day 0 using sterile PBS as the vehicle and observed for 10 days post injection. The treatment and dosing by group is shown in Table 35. The bacteria levels (10\hat{ }7 CFU/mouse) were chosen based on similar peer-reviewed articles studying SSTIs as well as systemic bacterial effects in mice of the same species and of similar age. Prior to injection, body hair was removed from the animals in the areas surrounding the injection site (dorsal surface). The animals were allowed adequate acclimation time, both before and after hair removal, to stabilize. Body weights were obtained and recorded on study Day 0. Pictures of the injection site/abscess were photographed once per day for all subjects in all groups. Abscesses present were measured once daily (length and width) using calipers. Body weights were measured once each morning for the duration of the study. Mortality and morbidity checks were performed twice a day (once in the morning and once in the evening) during business days and once on the weekends. Animals who experienced a 20% or greater loss in weight were deemed moribund suitable for euthanasia. All procedures abided by USDA guidelines for animal care and handling. Study design and animal usage were approved by the Institutional Animal Care and Use Committee (IACUC) in a USDA certified (84-R-0081) and OLAW assured facility (A4678-01).

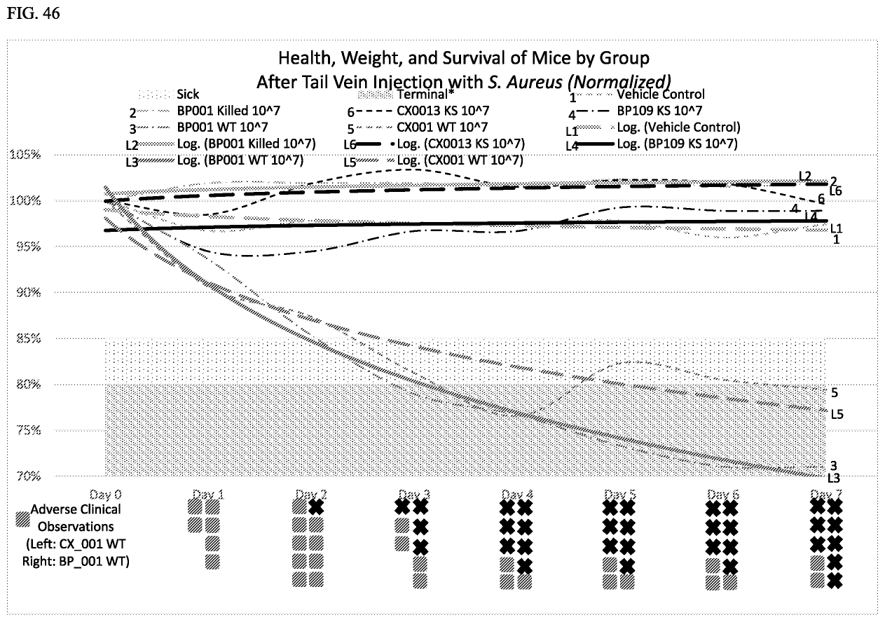

FIG. 47 shows a graph of animal health in the SSTI study as measured by abscess formation, or the lack thereof over the 10 day duration of the study. Mice in Groups 4 and 6, BP_109 and CX_013, respectively, maintained health over the course of this study, as compared to their wild type parent strains BP_001 and CX_013, respectively. Animals in the negative control Groups 1 (vehicle) and 2 (killed WT BP_001) all remained healthy throughout the study and are not shown.

On Study Day 1—the day following injection—clinical observations were normal for mice in the negative control Groups 1 and 2. Likewise, none of the mice in the KS groups—Groups 4 and 6—exhibited adverse clinical observations one day post injection, with the exception of one minor reaction. A small, light colored bump was observed on one mouse from Group 4, BP_109, on study Day 1. By study Day 2 the bump was no longer present on the Group 4 mouse, and all mice from the KS groups maintained good health with no adverse clinical observations for the remainder of the study. Images of the injection site were collected (FIGS. 1-2).

In contrast, half of the mice in the WT positive control groups began to exhibit signs of infection shortly after the onset of the study. Five of the ten mice from the WT positive control groups experienced abscess formation by study Day 1. This included two mice from Group 3, BP_001, and three mice from Group 5, CX_001. Signs of infection in the BP_001 group initially presented as yellow colored formations, which quickly progressed into large off-white colored abscesses surrounded by irritated red margins. Abscesses were present for the remainder of the study for both mice in Group 3.

The SSTIs in Group 5 presented as small red abscesses, and one mouse in Group 5 was observed to return to normal clinical observations by study Day 9. Abscesses were present for the duration of the study for the other two mice in Group 5.

The pre-dose mouse body weights ranged from 19.0 g to 24.1 g. All subjects maintained normal body weight for the duration of the study. Therefore, a hypothesis test for binomial distributions was used to compare the KS test subjects to the positive control subjects for significance. This was done by strain derivation; i.e. BP_109 was compared to BP_001 and CX_013 was compared to CX_001. Animals with abscess formation were assigned a value of 1 and those without abscess formation were assigned a value of 0, as shown in Table 36. As compared to WT SA subcutaneous injection, the BioPlx KS groups exhibited significantly fewer SSTIs (p<0.01).

Statistical Analysis

No weight deviation occurred for any of the groups involved in the study, so a dichotomous score was used to compare groups by an absolute measure. Any abscess formation throughout the study assigned a mouse a value of 1 and complete absence of abscess formation for the duration of the study assigned a mouse a value of 0. As such, the results are shown in Table 76.

TABLE 76
Dichotomous Score for Abscess Formation by Group per Mouse

| Group | Mouse | Mouse | Mouse | Mouse | Mouse | Group |
|---|---|---|---|---|---|---|
| Treatment | 1 | 2 | 3 | 4 | 5 | Score |
| BP_001 | 0 | 1 | 0 | 1 | 0 | 2/5 |
| BP_109 | 0 | 0 | 0 | 0 | 0 | 0/5 |
| CX_001 | 0 | 1 | 1 | 1 | 0 | 3/5 |
| CX_013 | 0 | 0 | 0 | 0 | 0 | 0/5 |
| Killed | 0 | 0 | 0 | 0 | 0 | 0/5 |
| BP_001 | | | | | | |

Abscess Formation = 1;
No Abscess Formation = 0

Abscess Formation=1; No Abscess Formation=0

The hypothesis test for binomial distributions was used to compare groups by parent/daughter strains. In other words, the analysis was used to compare BP_001 to BP_109 and CX_001 to CX_013 as the latter were derived from the former. Probability was assigned by the WT groups' presence of abscess formation, and alpha was set to 99% confidence.

The hypothesis test for binomial distributions determined that five out of five mice in the test group must be abscess free for both strains to achieve a 99% confidence. As all five mice from both test groups, BP_109 and CX_013, were completely abscess free, we may report that both test groups are significantly different to the comparative WT groups with a p-value<0.01.

In this SSTI study, all mice injected subcutaneously with SA KS organisms as well as negative controls were healthy and normal for the duration of the study, excluding one minor reaction on a test subject on study Day 1, which was resolved by the morning of Day 2. Half of the mice injected with WT SA organisms had abscess formations present for most of the study.

### Example 38. Plasmid Construction for p174 & p229

In this example, the plasmids p229 and p174 were made successfully and used to transform into S. agalactiae. The sequencing results showed no mutations.

Since the pRAB11 plasmid is a high copy vector with tight regulation of the genes downstream of the P_{xyl/tet} promoter, the system produces an easily detectable response from the genes downstream of the promoter. In plasmid p174 the toxin gene sprA1 was added to the pRAB11 plasmid and operably linked to P_{xyl/tet} for ATc-dependent TetR induction. In plasmid p229, green fluorescent protein (GFPmut2) was added to the pRAB11 plasmid and operably linked to P_{xyl/tet} for ATc-dependent TetR induction.

The pRAB11 plasmid is a high-copy expression vector used for anhydrotetracycline (ATc)-dependent expression of genes in either E. coli or Staph aureus. Plasmid pRAB11 was generated by adding another tetO operator to the TetR-regulated promoter, P_{xyl/tet}, in plasmid pRMC2. Helle, Leonie, et al., Microbiology 157.12 (2011): 3314-3323.

TetR is a transcriptional repressor protein that binds to DNA if the tetO sequence is present. The P_{XYL/tet} promoter in pRAB11 has two tetO sequences that flank the transcriptional start site which represses the transcription of any gene just downstream of the promoter. When ATc is added to the culture, it will bind to the repressor protein TetR and inhibit its ability to bind to tetO within the promoter. With the TetR proteins deactivated, the constitutive promoter is derepressed and is uninhibited when recruiting RNA polymerase to transcribe the putative toxin at a high rate.

For the construction of p174, the toxin gene sprA1 was added to pRAB11 and operably linked to P_{xyl/tet} for ATc-dependent TetR induction. The sprA1 gene is native to Staph aureus and is part of a type I toxin antitoxin system. The sprA1 gene codes for a membrane porin protein called PepA1, which accumulates in the cell's membrane and induces apoptosis in dividing cells. The sprA1 gene used here was PCR amplified from the genome of a 502a-like strain named in BioPlx's databases as BP_001.

For the construction of p229, a green fluorescent protein (GFPmut2) was added to pRAB11 behind the P_{xyl/tet} promoter for ATc-dependent expression. The expression of both proteins should go from a state of being transcriptionally repressed by the TetR protein to induced and expressed upon the addition of ATc to the system.

Table 77 shows the single stranded DNA sequences for the primers used during the construction or sequencing of plasmid p174 and p229. All of the sequences are in the 5 prime to 3 prime direction.

TABLE 77
Primers Used to Make Plasmids p174 and p229

| | Primer | |
|---|---|---|
| Plasmid | Name | Primer Sequence (5′-->3′) |
| p174 | BP_672 | gagtatgatggtaccgttaacagatctgagc |
| | | CGCAGAGAGGAGGTGTATAAGGTG (SEQ |
| | | ID NO: 278) |
| | BP_677 | gttgtaaaacgacggccagtgCCCGGGCTCA |
| | | GCTATTATCA (SEQ ID NO: 282) |
| | BP_670 | GCTCAGATCTGTTAACGGTACCATCATACTC |
| | | (SEQ ID NO: 276) |
| | BP_671 | CACTGGCCGTCGTTTTACAAC (SEQ ID |
| | | NO: 277) |
| p229 | BP_717 | ACTCTTTGAAGTCATTCTTTACAGGAG |
| | | (SEQ ID NO: 580) |
| | DR_244 | CATCACCTTATACACCTCCTCTCTGCGG |
| | | (SEQ ID NO: 244) |
| | DR_476 | CCGCAGAGAGGAGGTGTATAAGGTGATGAGT |
| | | AAAGGAGAAGAACTTTTCAC (SEQ ID NO: |
| | | 597) |
| | DR_247 | CAATTTTTATTGGTGCGGCTATATGTCACTTA |
| | | TTTGTATAGTTCATCCATGCCATGTG (SEQ |
| | | ID NO: 598) |
| | BP_718 | CTCCTGTAAAGAATGACTTCAAAGAGT (SEQ |
| | | ID NO: 581) |
| | DR_245 | GTGACATATAGCCGCACCAATAAAAATTGATA |
| | | ATAGCTGAGCC (SEQ ID NO: 599) |

Table 78 shows the DNA sequences used in the construction of p174 and p229. The sequences represent one strand of the double stranded DNA fragments.

TABLE 78
Sequences of PCR Fragments Inserted into Plasmid

| Plasmid | Name | Seq. ID | Sequence |
|---|---|---|---|
| p174 | sprA1 | BP_DNA_150 | CGCAGAGAGGAGGTGTATAAGGTGATGCTTATT |
| | | | TTCGTTCACATCATAGCACCAGTCATCAGTGGC |
| | | | TGTGCCATTGCGTTTTTTTCTTATTGGCTAAGTA |
| | | | GACGCAATACAAAATAGGTGACATATAGCCGC |
| | | | ACCAATAAAAAT (SEQ NO: 600) |
| p229 | GFPmut2 | BP_DNA_077 | ATGAGTAAAGGAGAAGAACTTTTCACTGGAGTT |
| | | | GTCCCAATTCTTGTTGAATTAGATGGTGATGTTA |
| | | | ATGGGCACAAATTTTCTGTCAGTGGAGAGGGTG |
| | | | AAGGTGATGCAACATACGGAAAACTTACCCTTA |
| | | | AATTTATTTGCACTACTGGAAAACTACCTGTTCC |
| | | | ATGGCCAACACTTGTCACTACTTTCGCGTATGGT |
| | | | CTTCAATGCTTTGCGAGATACCCAGATCATATG |
| | | | AAACAGCATGACTTTTTCAAGAGTGCCATGCCC |
| | | | GAAGGTTATGTACAGGAAAGAACTATATTTTTC |
| | | | AAAGATGACGGGAACTACAAGACACGTGCTGA |
| | | | AGTCAAGTTTGAAGGTGATACCCTTGTTAATAG |
| | | | AATCGAGTTAAAAGGTATTGATTTTAAAGAAGA |
| | | | TGGAAACATTCTTGGACACAAATTGGAATACAA |
| | | | CTATAACTCACACAATGTATACATCATGGCAGA |
| | | | CAAACAAAAGAATGGAATCAAAGTTAACTTCA |
| | | | AAATTAGACACAACATTGAAGATGGAAGCGTTC |
| | | | AACTAGCAGACCATTATCAACAAAATACTCCAA |
| | | | TTGGCGATGGCCCTGTCCTTTTACCAGACAACC |
| | | | ATTACCTGTCCACACAATCTGCCCTTTCGAAAG |
| | | | ATCCCAACGAAAAGAGAGACCACATGGTCCTTC |
| | | | TTGAGTTTGTAACAGCTGCTGGGATTACACATG |
| | | | GCATGGATGAACTATACAAATAA (SEQ NO: |
| | | | 381) |

The following PCR reactions were performed using Q5 High Fidelity Hot Start Master Mix (NEB) per the manufacturer's instructions.
BP_DNA_095—p151 Backbone Fragment (p174)
   BP_670/BP_671
BP_DNA_095—p174 Backbone Fragment (p229)
   DR_244/DR_245
BP_DNA_sprA1 (Inserted sequence) (p174)
   BP_672/BP_677
BP_DNA_077—GFPmut2 (Inserted sequence) (p229)
   DR_476/DR_247

The above PCR fragments were checked on a 1% agarose gel to confirm a clean band, and then purified using a Qiaquick PCR Purification Kit (Qigagen) per the manufacturer's instructions. The p174 fragment was treated with DpnI (NEB) to remove the pRAB11 plasmid used as the template for the PCR, and purified again using the PCR Cleanup Kit (NEB) per the manufacturer's instructions. The DNA fragments were used in a Gibson Assembly (NEB) to create a circular plasmid per the manufacturer's instructions. The assembled plasmid was then transformed into IM08B, plated on LB (carb), and incubated overnight at 37° C. The following day, colonies were screened for fully assembled plasmids by colony PCR to check for the presence of the GFP or sprA1 on the pRAB11 plasmid within the colony. Three positive colonies were picked, grown overnight in 5 mL of LB (plus carbenicillin, 100 ug/mL), and the plasmid was extracted using the ZymoPURE plasmid miniprep kit per the manufacturer's instructions. The plasmid was then sequenced to confirm the DNA sequence of the GFPmut2 or sprA1 gene. The sequencing was aligned in silico using the sequence alignment tool in Benchling. One of each of the colonies whose sequencing alignment that showed a perfect alignment to the reference map's sequence was picked and stocked in the plasmid database.

### Example 39. Transformation of Electrocompetent Streptococcus agalactiae Cells

Streptococcus agalactiae was transformed by a variation of procedures from Framson et al. and Duny et al. (Framson, et al., Appl. Environ. Microbiol. 1997, 63 (9), 3539-3547, Dunny et al., Appl. Environ. Microbiol. 1991, 57 (4), 1194-1201).

Briefly, the electrocompetent cell protocol starts by inoculating a single overnight culture of S. agalactiae A909 (BPST_002) in M9 Media with 1% Casamino Acids and 0.3% Yeast Extract (M9-YE) and incubating overnight at 37° C. The next day, that culture was used to inoculate a larger volume of the same media but with 1.2% glycine. The new culture was statically incubated at 37° C. for 12 to 15 h. Glycine disrupts the biosynthesis of the peptidoglycan cell wall by replacing the L-alanine in the peptide crosslinker. This causes pore formation in the electrocompetent cells and therefore increases the likelihood of DNA uptake during transformation. After the incubation period, the culture with glycine will be added into a larger volume of fresh M9-YE+1.2% glycine and incubated for 1 h at 37° C. After the growth period, the OD was checked and found to be in the target range of 0.1-0.25 OD. After the culture reached the target OD, the cells were pelleted by centrifuging the culture and the resulting supernatant was removed. The cell pellet was resuspended in an osmoprotectant solution (0.625 M Sucrose, pH 4), pelleted again through centrifugation and the supernatant removed. The cells were resuspended in a small volume of the osmoprotectant solution. After the final resuspension, the cells were either chilled on ice for 30 to 60 minutes and used for electroporation, or immediately stored in the −80° C. freezer.

The electroporation protocol followed the procedure by Duny et al. but used recovery media from the Framson et al. protocol.

Briefly, competent S. agalactiae cells were thawed on ice, transferred to a 2-mm electroporation cuvette where at least 300 ng of plasmid DNA was added directly to the competent cells, and the cells are electroporated at 2.0 kV with a 200Ω resistance. Afterwards, the cuvette was briefly placed on ice, 0.5 M sucrose in THB is added to the cells and the suspension is transferred to a culture tube. The transformation is statically recovered at 37° C. for 1 hr before being plated on THB agar plates with the appropriate antibiotic selection. The plates are incubated overnight at 37° C. and the presence of colonies indicates that plasmid has been taken up by S. agalactiae.

### Example 40. Toxin Efficacy Test in S. agalactiae Using Inducible Gene Expression

The putative Staphylococcus aureus toxin gene sprA1 under the control of the P_{XYL/Tet} promoter on the pRAB11 vector was transformed into Streptococcus agalactiae A909 (BPST_002) by the method of Example 39.

In the present example the ability of the sprA1 toxin gene from Staphylococcus aureus (S. aureus) to cause cell death or prevent cell growth when expressed from a pRAB11 plasmid transformed into Streptococcus agalactiae (S. agalactiae) was tested. A strong inducible and tightly controlled promoter system, P_{XYL/Tet} on pRAB11 was employed. The effect of sprA1 overexpression on the growth of S. agalactiae was observed by measuring the optical density (OD) of the culture over the growth period.

Overexpression of the sprA1 gene prevented growth of the BPST_002 cell cultures, indicating the production of PepA1 functions as a bacteriostatic toxin to the host cells. To verify the P_{XYL/Tet} promoter, a plasmid with a GFP operably linked to the P_{XYL/Tet} promoter was also transformed into S. agalactiae A909 (BPST_002). Induction of the GFP-containing plasmid showed a 10-fold increase in the amount of fluorescence between induced cultures and uninduced cultures.

pRAB11 plasmids p174 and p229 containing a toxin and green fluorescence protein (GFP), respectively, under the control of the P_{XYL/Tet} promoter system were transformed into BPST_002.

In plasmid p174, the sprA1 gene was added directly after the promoter system. The toxin is native to Staph aureus, and is part of a type I toxin antitoxin system. The sprA1 gene used here was PCR amplified from the genome of a Staphylococcus aureus 502a-like strain BP_001.

In plasmid p229, a GFPmut2 was added to pRAB11 behind the P_{xyl/tet} promoter. The expression of both proteins was expected to go from a state of being transcriptionally repressed by the TetR protein to induced and expressed upon the addition of ATc to the system.

This system was used to test the effect of overexpression of the sprA1 toxin, PepA1, on the growth of BPST_002 (S. agalactiae A909). The sprA1 gene codes for a membrane porin protein called PepA1, which accumulates in the cell's membrane and induces apoptosis in dividing cells. This effect was expected to cause cell death or failure of cells to grow in cultures induced with Atc, as measured by OD600. To confirm the effectiveness of the PXYLutet promoter, the fluorescence of induced and uninduced cultures was measured using a plate reader.

Table 79 shows the plasmid numbers and descriptions that were transformed into BPST_002.

TABLE 79
Plasmids Transformed into Streptococcus agalactiae BPST_002

| Number | Name | Description |
|---|---|---|
| p174 | pRAB11_Ptet- | sprA1 toxin gene (without antitoxin |
| | sprA1 | sequence) under control of tetracycline- |
| | | inducible promoter. The gene includes some |
| | | sequence upstream of the start codon. |
| p229 | pRAB11_P(xyl- | Green fluorescent protein gene under control |
| | tet)-GFPmut2 | of anhydrotetracycline-inducible promoter |

Transformation and PCR Screen

The plasmids were electroporated into BPST_002 electrocompetent cells and colonies were PCR screened for the presence of the plasmid using DR_216/DR_217. Plasmids p229 and p174 were transformed into the S. agalactiae BPST_002 electrocompetent cells using the protocol above. The transformation was recovered statically at 37° C. for 1 hr and plated on THB agar plates with 1 ug/mL of chloramphenicol. The plates were incubated for 16-24 hrs. When colonies were visible, a sterile inoculation loop was employed to pick single colonies from each transformation and restreak for single colony isolation on fresh THB agar plates with 1 μg/mL of chloramphenicol. The plates were incubated at 37° C. for 12-16 hrs.

The following day, colonies were PCR screened on new streak plates for the presence of the plasmid using DR_215 (SEQ ID NO: 582)/DR_216 (SEQ ID NO: 583). PCR products were run on a 1% agarose gel to check for colonies that are positive for the integration. If all colonies are positive for the presence of the plasmid, the streak plate was used to start cultures for growth assays.

Growth Assay with Stationary Phase Cultures
1. Start three 5 mL THB+chloramphenicol (1 μg/mL) culture for each plasmid to be tested from a single colony on fresh agar plates. Statically incubate for 8 hr at 37° C.
2. After the incubation period, measure the OD600 of the cultures.
3. Add 5 μL of anhydrotetracycline (ATc) (1 ug/mL) to two of the three samples. The unspiked sample is the control.
4. Statically incubate culture tubes at 37° C. for 1 hour.
5. After the incubation period, measure the OD600 of the cultures.
6. Enter recorded ODs in a table and plot the data on a graph to show the growth curves for all of the strains tested.

Growth Assay with Exponential Phase Cultures
1. Start three 5 mL THB+chloramphenicol (1 μg/mL) culture for each plasmid to be tested from a single colony on fresh agar plates. Statically incubate for 8 hr at 37° C.
2. After the incubation period, measure the OD600 of the cultures.
3. Add 500 μL of cultures to 4.5 mL of fresh THB+chloramphenicol (1 μg/mL), briefly vortex to mix the culture.
4. Remove 500 μL of each culture and measure the OD600.
5. Add 4.5 μL of anhydrotetracycline (ATc) (1 mg/mL) to two of the three samples. The unspiked sample is the control.
6. Immediately after the addition of the ATc and before putting the tubes in the 37° C. incubator, briefly vortex to mix the culture.
7. Statically incubate culture tubes at 37° C. for 1 hour.
8. After 1 hour measure and record the OD600 readings,
9. Place cultures back in the 37° C. incubator and measure and record the OD600 values every hour for a total of 3 hrs.

Fluorescence Sample Preparation and Measurements
10. After 3 hrs of incubation, spin down the p229 in BPST_002 cultures for 5 minutes at 3500 rpm.
11. Remove the supernatant and add 5 mL of PBS. Resuspended the cultures by briefly vortexing.
12. Centrifuge cultures again for 5 minutes at 2800×g.
13. Remove the supernatant and resuspend cell pellet in 1 mL of PBS.
14. Add 200 uL of each cell suspension to a 96-well plate (Greiner Bio, Part #655900) in triplicate. Include PBS in triplicate as a blank.
15. Read the plate with the following settings:
   a. Ex: 485/20
   b. Em: 530/25
   c. Sensitivity: 80
16. Subtract the blank reading from the experimental samples and record all values.

### Results:

Both plasmids p174 and p229 were successfully transformed into Streptococcus agalactiae BPST_002 and PCR confirmed with DR_215 and DR_216. Growth assays were performed on a single day with cultures started directly from a single colony. The assays were performed in the exact same manner each time according to the protocol described above.

Table 80 shows the OD₆₀₀ readings for p174 & p229 in BPST_002 grown in THB. The OD600 for induced cultures where ATc was added to induce the expression of the sprA1 toxin or GFP reporter gene, were compared to uninduced cultures (control, no ATc).

TABLE 80
OD Values of p174 & p229 in BPST_002 (+/−ATc) over 3 hours

Time (hours)

| Sample Name | 0 | 1 | 2 | 3 |
|---|---|---|---|---|
| p174 + ATc #1 | 0.22 | 0.23 | 0.23 | 0.24 |
| p174 + ATc #2 | 0.25 | 0.23 | 0.23 | 0.22 |
| p174 (control) | 0.26 | 0.7 | 2.4 | 2.4 |
| p229 + ATc #1 | 0.28 | 0.5 | 1.0 | 1.4 |
| p229 + ATc #2 | 0.29 | 0.5 | 1.2 | 1.6 |
| p229 (control) | 0.27 | 0.7 | 2.1 | 2.3 |

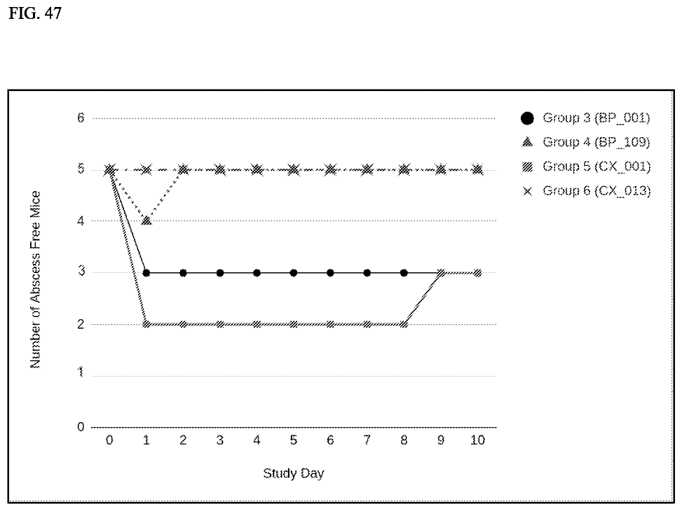

The data from Table 80 is plotted on a graph in FIG. 48. FIG. 48 shows a graph of OD600 growth curves over 3 hours for Streptococcus agalactiae (BPST_002) transformed with plasmids p174 (sprA1) or p229 (GFP). The starting cultures were inoculated at a 1:10 dilution from stationary phase cultures. The t=0 hr OD was taken before ATc induction. The dashed line represents the cultures that were induced with ATc and the solid line represents control cultures. overexpression of sprA1 toxin gene is able to inhibit S. agalactiae cell growth in exponential phase All data points represent single cultures.

The results show that overexpression of sprA1 toxin gene is able to inhibit S. agalactiae cell growth in exponential phase. The OD600 values of the ATc spiked samples did not increase after the addition of ATc, while the control samples continued to grow. This indicates that the sprA1 gene from S. aureus is capable of inhibiting growth and possibly killing S. agalactiae cells when overexpressed.

To show that ATc is not inherently toxic to the cells and therefore responsible for the inhibition of cell growth, cultures of wild-type BPST_002 were grown overnight. One culture was induced with ATc and the resulting OD was compared to the non-induced culture. The ATc culture had a 10% higher OD600 as compared to the control culture (data not shown). Therefore, the addition of ATc at a concentration of 1 ug/mL was not toxic to BPST_002 cell growth.

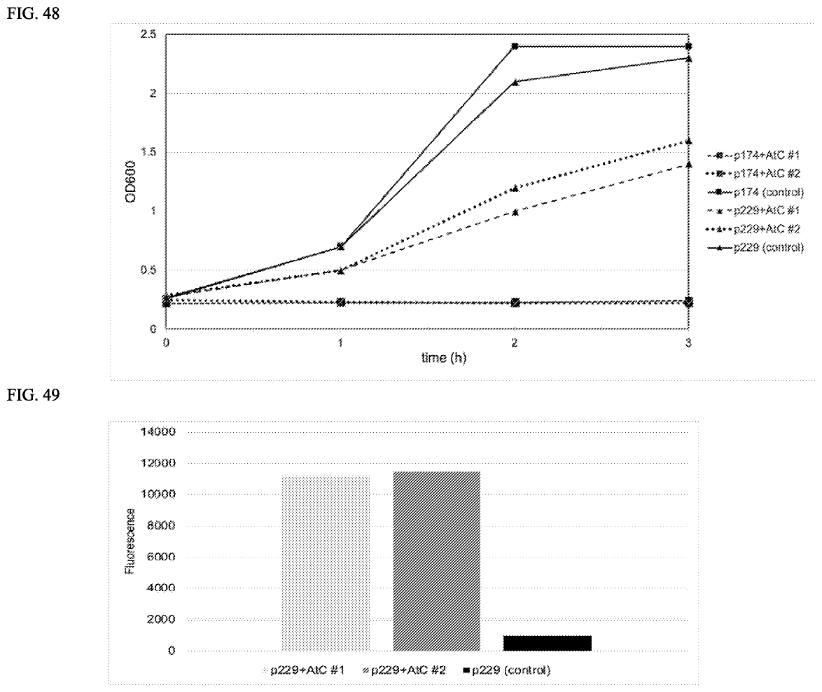

FIG. 49 shows a bar graph of fluorescence values at 3 hours after induction of Streptococcus agalactiae (BPST_002) transformed with plasmid p229 (GFP). The starting cultures were inoculated at a 1:10 dilution from stationary phase cultures. Cultures were grown in duplicate and fluorescence readings were performed in triplicate. Increased fluorescent values of induced p229 cultures indicate the ability of the P_{XYL/Tet} promoter system of pRAB11 to function as an ATc inducible promoter in S. agalactiae.

### Example 41. Stability of a Mixture of Staphylococcus aureus, Streptococcus agalactiae and Escherichia coli

The stability of a mixture of synthetic Staphylococcus aureus (BP_123), synthetic Escherichia coli (BPEC_006), and Streptococcus agalactiae (BPST_002, WT A909) in PBS was determined.

Cell suspensions of BP_123, BPST_002 and BPEC_006 in PBS were relatively stable after 24 h storage at 4° C. as assessed by CFU plating. After 24 h, BP_123 decreased by 25% in a mixture with BPST_002 and BPEC_009, but also decreased in a suspension that contained only BP_123. BPST_002 and BPEC_009 remained within +/−10% of the original t=0 samples in the cell suspension mixture with all 3 bacteria types. Colonies were visually differentiated by growth characteristics on TSB and supported by PCR strain screen data.

Bovine mastitis can be caused by three main bacterial species; Staphylococcus aureus, Streptococcus agalactiae and Escherichia coli. These bacteria can live naturally within the bovine microbiome or environment but can cause mastitis if an opportunistic infection occurs in the udder.

Synthetic strains of all of these species can be prepared by genomically integrating a safety switch using kill switch technology in order to cause immediate bacterial cell death upon entering the bloodstream or tissue.

A live biotherapeutic composition containing a mixture of all three bacterial types must ensure that the viability of each of the bacteria remains stable when mixed together. This example assesses the stability of S. aureus (BP_123), S. agalactiae (BPST_002) and E. coli (BPEC_006) when suspended in phosphate buffered saline (PBS) together for future use as a biotherapeutic intervention for bovine mastitis.

Briefly, BP_123, BPEC_006 and BPST_002 were grown in overnight overnight cultures. The following day the cells were harvested, washed three times in PBS and concentrated. The concentration of viable colony forming units (CFUs) was determined by performing a serial dilution of the cell suspension, plating several different dilutions on non-selective agar plates, and counting the colonies the following day to calculate the cell concentration. The washed cultures were then resuspended in an appropriate volume of PBS to reach the target concentration of 1×10⁷ CFU/mL. The stability suspensions were plated on TSB plates and the suspensions were stored at 4° C. After 24 hrs of storage the stability suspensions were plated again and the final CFU/mL compared to the t=0 CFU/mL.

Table 81 shows the strain numbers and description of strains that were used in the stability study.

TABLE 81
Strains in Stability Study

| Number | Bacteria | Strain Description |
|---|---|---|
| BPST_002 | S. agalactiae | Strain A909, wild-type |
| BPEC_006 | E. coli | E. coli isolated from |
| | | bovine sample (Udder Health Systems, |
| | | Inc.) Genetically modified: |
| | | DuidA::tetR_Pxyl/tet-sprA1_kanR |
| BP_123 | S. aureus | Strain 502a, Genetically modified: |
| | | ΔsprA1; isdB::sprA1 |

Table 82 shows stability suspension mixtures, the final target concentration and final volume of PBS.

TABLE 82
Stability Suspension Mixtures of S. agalactiae,
E. coli, and S. aureus

| | Stability | Target | Final Volume |
|---|---|---|---|
| | Samples | Concentration | (uL) |
| A | BP_123 | 1.00E+07 | 5000 |
| B | BPST_002 | 1.00E+07 | 5000 |
| C | BPEC_006 | 1.00E+07 | 5000 |
| D | BP_123 | 1.00E+07 | 5000 |
| | BPST_002 | 1.00E+07 | 5000 |
| | BPEC_006 | 1.00E+07 | 5000 |
| E | BP_123 | 1.00E+07 | 5000 |
| | BPST_002 | 1.00E+07 | 5000 |
| F | BP_123 | 1.00E+07 | 5000 |
| | BPEC_006 | 1.00E+07 | 5000 |
| G | BPST_002 | 1.00E+07 | 5000 |
| | BPEC_006 | 1.00E+07 | 5000 |

A 10⁻⁵ dilution of Stability Suspension D containing BP_123, BPST_002 and BPEC_006 was plated on TSB. Colonies were visibly different so BP_123 colonies could be differentiated from BPST_002 and BPEC_006 and vice versa.

Strain identities were confirmed using PCR. The PCRs products were run on a 1% agarose gel of the strain screen from lysed colonies from stability suspension D TSB plate. All colonies were screened from a single 10⁻⁵ dilution plate using the SA lysis procedure. Visibly like colonies were grouped together and the 3 PCRs were run on all of the lysates. Primers are shown in Table 83.

TABLE 83
PCR Band Size and Primer Details for Strain Screen

| | | | | PCR | |
|---|---|---|---|---|---|
| | | PCR | Primer Sequence | band | |
| Number | Bacteria | Primers | (5′-->3′) | size (bp) | Target Area |
| BP_123 | S. aureus | DR_254 | ATGCTTATTTTCGTTCA | 1391 | sprA1 |
| | | | CATCATAGCACCAGTC | | integration |
| | | | ATCAGTG | | site to DNA |
| | | DR_534 | CAGCTGTTGATAATGCC | | outside of |
| | | | ATTTTTGCACGAG | | integration |
| | | | | | area |
| BPEC_ | E. coli | DR_372 | GCCATCTGTAAATCTTG | 2114 | sprA1 |
| 006 | | | CGCCATTAGTCC | | integration |
| | | DR_254 | ATGCTTATTTTCGTTCA | | site to DNA |
| | | | CATCATAGCACCAGTC | | outside of |
| | | | ATCAGTG | | integration |
| | | | | | area |
| BPST_ | S. | BM_152 | AGGAATACCAGGCGAT | 952 | dltS gene² |
| 002 | agalactiae | | GAACCGAT | | |
| | | BM_153 | TGCTCTAATTCTCCCCT | | |
| | | | TATGGC | | |

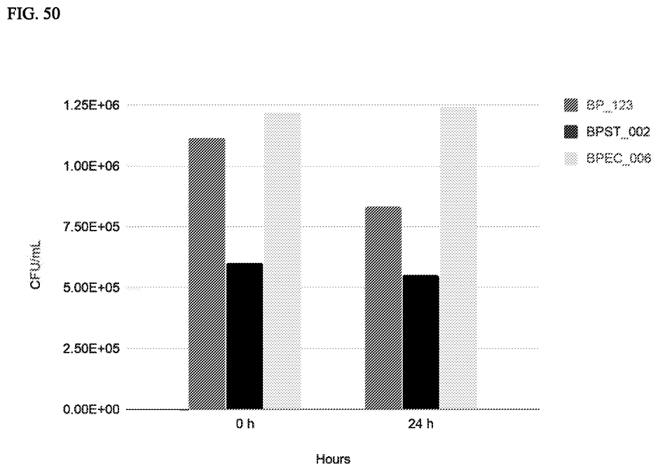

Stability results are shown in FIG. 50 showing a bar graph calculated from the CFU/mL data of Stability Suspension D containing BP_123, BPST_002, BPEC_006 at 0 and 24 hours. All dilutions were plated in duplicate on TSB plates. CFU/mL data was calculated from the 10⁻⁴ dilution.

The observed CFU/mL at t=0 and 24 h supports the stability of cell suspensions containing a mixture of S. aureus, S. agalactiae and E. coli. In stability suspension D, CFU/mL of BPST_002 and BPEC_006 remained stable after a period of 24 h but BP_123 viability decreased by roughly 25% as seen in FIG. 50. Cell suspension A, containing only BP_123, also decreased significantly from t=0 h. Based on this data, BP_123 decreased independently of being mixed with BPEC_006 or BPST_002. The CFU/mL of BPST_002 and BPEC_006 in stability suspension D were comparable to stability suspensions B and C which contained only one type of bacteria. This also leads to the conclusion that a mixed cell population does not influence the CFU/mL of different bacterial types which is important for the development of a biotherapeutic intervention for bovine mastitis.

What is claimed is:

1. A live biotherapeutic composition for treatment or prevention of bovine, caprine, ovine, or porcine mastitis and/or intramammary infection comprising at least one synthetic microorganism, and a pharmaceutically acceptable carrier, wherein the synthetic microorganism comprises a recombinant nucleotide comprising
at least one kill switch molecular modification comprising
a first cell death gene operatively associated with
a first regulatory region comprising an inducible first promoter, wherein
the first inducible promoter exhibits conditionally high level gene expression of the recombinant nucleotide in response to exposure to blood, serum, plasma, interstitial fluid, synovial fluid, or contaminated cerebral spinal fluid of at least three fold increase of basal productivity.

2. The composition of claim 1, wherein the synthetic microorganism further comprises
at least a second molecular modification (expression clamp) comprising
an antitoxin gene specific for the first cell death gene, wherein the antitoxin gene is operably associated with
a second regulatory region comprising a second promoter which is active (constitutive) upon dermal or mucosal colonization or in a complete media, but is not induced, induced less than 1.5-fold, or is repressed after exposure to blood, serum or plasma for at least 30 minutes.

3. The composition of claim 1 or 2, wherein the synthetic microorganism is derived from a target microorganism having the same genus and species as an undesirable microorganism causing bovine, caprine, ovine, or porcine mastitis.

4. The composition of claim 1 or 2, wherein the first promoter is upregulated by at least 5-fold, at least 10-fold, at least 20-fold, at least 50-fold, or at least 100-fold within at least 30 min, 60 min, 90 min, 120 min, 180 min, 240 min, 300 min, or at least 360 min following exposure to blood, serum, plasma, or interstitial fluid.

5. The composition of claim 1 or 2, wherein the first promoter is not induced, induced less than 1.5 fold, or is repressed in the absence of blood, serum, plasma, interstitial fluid, synovial fluid, or contaminated cerebral spinal fluid.

6. The composition of claim 2, wherein the second regulatory region comprising a second promoter is active upon dermal or mucosal colonization or in TSB media, but is repressed at least 2 fold upon exposure to blood, serum, plasma, or interstitial fluid after a period of time selected from the group consisting of the group consisting of at least 30 min, 60 min, 90 min, 120 min, 180 min, 240 min, 300 min, and at least 360 min.

7. The composition of any one of claims 1 to 6, wherein measurable average cell death of the synthetic microorganism occurs within at least a preset period of time following induction of the first promoter.

8. The composition of claim 7, wherein the measurable average cell death occurs within at least a preset period of time selected from the group consisting of within at least 1, 5, 15, 30, 60, 90, 120, 180, 240, 300, or 360 min minutes following exposure to blood, serum, plasma, or interstitial fluid.

9. The composition of claim 8, wherein the measurable average cell death is at least a 50% cfu, at least 70%, at least 80%, at least 90%, at least 95%, at least 99%, at least 99.5%, at least 99.8%, or at least 99.9% cfu count reduction following the preset period of time.

10. The composition of any one of claims 1 to 9, wherein the kill switch molecular modification reduces or prevents infectious growth of the synthetic microorganism under systemic conditions in the subject.

11. The composition of claim 1 or 2, wherein the at least one molecular modification is integrated to a chromosome of the synthetic microorganism.

12. The composition of claim 3, wherein the target microorganism is susceptible to at least one antimicrobial agent.

13. The composition of claim 12, wherein the target microorganism is selected from a bacterial and/or yeast target microorganism.

14. The composition of claim 13, wherein the target microorganism is a bacterial species capable of colonizing a dermal and/or mucosal niche and is a member of a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

15. The composition of claim 14, wherein synthetic microorganism is derived from a Staphylococcus aureus strain.

16. The composition of claim 13, wherein the target microorganism is a yeast.

17. The composition of claim 16, wherein the target microorganism is a yeast species capable of colonizing a dermal and/or mucosal niche and is a member of a genus selected from the group consisting of Candida and Cryptococcus.

18. The composition of claim 15, wherein the cell death gene is selected from the group consisting of sprA1, sprA2, kpn1, sma1, sprG, relF, rsaE, yoeB, mazF, yefM, or lysostaphin toxin gene.

19. The composition of claim 18, wherein the cell death gene comprises a nucleotide sequence selected from the group consisting of SEQ ID NOs: 122, 124, 125, 126, 127, 128, 274, 275, 284, 286, 288, 290, 315, and 317, or a substantially identical nucleotide sequence.

20. The composition of claim 18 or 19, wherein the inducible first promoter comprises or is derived from a gene selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

21. The composition of claim 20, wherein the first promoter comprises a nucleotide sequence selected from the group consisting of SEQ ID NO: 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical nucleotide sequence thereof.

22. The composition of any one of claims 18 to 21, wherein the antitoxin gene encodes an antisense RNA sequence capable of hybridizing with at least a portion of the first cell death gene.

23. The composition of claim 22, wherein the antitoxin gene is selected from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively.

24. The composition of claim 23, wherein the antitoxin gene comprises a nucleotide sequence selected from the group consisting of SEQ ID NOs: 273, 306, 307, 308, 309, 310, 311, 312, 314, 319, 322, 342, 347, 362, 364, 368, 373, 374, 375, 376, 377, and 378, or a substantially identical nucleotide sequence.

25. The composition of claim 23 or 24, wherein the second promoter comprises or is derived from a gene selected from the group consisting of clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR family transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

26. The composition of claim 25, wherein the second promoter is a P_{clfB} (clumping factor B) and comprises a nucleotide sequence of SEQ ID NO: 117, 118, 129 or 130, or a substantially identical nucleotide sequence thereof.

27. The composition according to any one of claims 1 to 26, further comprising a molecular modification selected from the group consisting of a virulence block molecular modification, and nanofactory molecular modification.

28. The composition of claim 27, wherein the virulence block molecular modification prevents horizontal gene transfer of genetic material from the undesirable microorganism.

29. The composition of claim 27, wherein the nanofactory molecular modification comprises an insertion of a gene that encodes, a knock out of a gene that encodes, or a genetic modification of a gene that encodes a product selected from the group consisting of an enzyme, amino acid, metabolic intermediate, and a small molecule.

30. The composition comprising of any one of claims 1 to 29, wherein the pharmaceutically acceptable carrier includes a diluent, emollient, binder, excipient, lubricant, film-forming agent, sealant, colorant, dye, wetting agent, preservative, buffer, or absorbent, or a combination thereof.

31. The composition of claim 30, further comprising a nutrient, prebiotic, commensal, and/or probiotic bacterial species.

32. A single dose unit comprising the composition of claim 30 or 31.

33. The single dose unit of claim 32, comprising at least at least 10⁵, at least 10⁶, at least 10⁷, at least 10⁸, at least 10⁹, at least 10¹⁰ CFU, or at least 10¹¹ of the synthetic microorganism and a pharmaceutically acceptable carrier, optionally formulated for topical administration or intramammary administration.

34. The composition of any one of claims 1 to 31 or the single dose unit of any one of claims 32 to 33 for use in the manufacture of a medicament for eliminating and preventing the recurrence of bovine, caprine, porcine, or ovine mastitis.

35. The composition of any one of claims 1 to 31 or the single dose unit of any one of claims 32 to 34, comprising two or more, three or more, four or more, five or more, six or more, seven or more, eight or more, nine or more, or ten or more synthetic microorganisms.

36. The composition or single dose unit of any one of claims 1 to 35, comprising three or more synthetic microorganisms derived from target microorganisms including each of a Staphylococci species, a Streptococci species, and an Escherichia coli species.

37. The composition of claim 36, wherein the target Staphylococcus species is selected from the group consisting of a catalase-positive Staphylococcus species and a coagulase-negative Staphylococcus species.

38. The composition of claim 36 or 37, wherein the target Staphylococcus species is selected from the group consisting of Staphylococcus aureus, S. epidermidis, S. chromogenes, S. simulans, S. saprophyticus, S. sciuri, S. haemolyticus, and S. hyicus.

39. The composition of any one of claims 36 to 38, wherein the target Streptococci species is a Group A, Group B or Group C/G species.

40. The composition of any one of claims 36 to 39, wherein the target Streptococci species is selected from the group consisting of Streptococcus uberis, Streptococcus agalactiae, Streptococcus dysgalactiae, and Streptococcus pyogenes.

41. The composition of any one of claims 36 to 40, wherein the E. coli species is a Mammary Pathogenic Escherichia coli (MPEC) species.

42. A method for treating, preventing, or preventing the recurrence of bovine, caprine, ovine, or porcine mastitis or intramammary infection associated with an undesirable microorganism in a subject hosting a microbiome, comprising:
a. decolonizing the bovine, caprine, or ovine host microbiome; and
b. durably replacing the undesirable microorganism by administering to the subject a biotherapeutic composition comprising a synthetic microorganism comprising at least one element imparting a non-native attribute, wherein the synthetic microorganism is capable of durably integrating to the host microbiome, and occupying the same niche in the host microbiome as the undesirable microorganism.

43. The method of claim 42, wherein the decolonizing is performed on at least one site in the bovine, caprine, or ovine subject to substantially reduce or eliminate the detectable presence of the undesirable microorganism from the at least one site.

44. The method of claim 43, wherein the detectable presence of the undesirable microorganism is determined by a method comprising a phenotypic method and/or a genotypic method, optionally wherein the phenotypic method is selected from the group consisting of biochemical reactions, serological reactions, susceptibility to anti-microbial agents, susceptibility to phages, susceptibility to bacteriocins, and/or profile of cell proteins, and optionally
wherein the genotypic method is selected from the group consisting of hybridization, plasmids profile, analysis of plasmid polymorphism, restriction enzymes digest, reaction and separation by Pulsed-Field Gel Electrophoresis (PFGE), ribotyping, polymerase chain reaction (PCR) and its variants, Ligase Chain Reaction (LCR), and Transcription-based Amplification System (TAS).

45. The method of claim 43, wherein the niche is an intramammary, dermal, or mucosal environment that allows stable colonization of the undesirable microorganism at the at least one site.

46. The method of claim 45, wherein the ability to durably integrate to the host microbiome is determined by detectable presence of the synthetic microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

47. The method of claim 46, wherein the ability to durably replace the undesirable microorganism is determined by the absence of detectable presence of the undesirable microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

48. The method of claim 47, wherein the ability to occupy the same niche is determined by absence of co-colonization of the undesirable microorganism and the synthetic microorganism at the at least one site after the administering step,
optionally wherein the absence of co-colonization is determined at least one week, at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

49. The method of claim 42, wherein the at least one element imparting the non-native attribute is durably incorporated to the synthetic microorganism.

50. The method of claim 49, wherein the at least one element imparting the non-native attribute is durably incorporated to the host microbiome via the synthetic microorganism.

51. The method of claim 50, wherein the at least one element imparting the non-native attribute is selected from the group consisting of kill switch molecular modification, virulence block molecular modification, metabolic modification, and nano factory molecular modification.

52. The method of claim 51, wherein the molecular modification is integrated to a chromosome of the synthetic microorganism.

53. The method of claim 51, wherein the synthetic microorganism comprises a virulence block molecular modification that prevents horizontal gene transfer of genetic material from the undesirable microorganism.

54. The method of claim 51, wherein the synthetic microorganism comprises a kill switch molecular modification that reduces or prevents infectious growth of the synthetic microorganism under systemic conditions in the subject.

55. The method of claim 51, wherein the synthetic microorganism is derived from a target microorganism having the same genus and species as the undesirable microorganism.

56. The method of claim 51, wherein the synthetic microorganism is derived from a target microorganism that has the ability to biomically integrate with the decolonized host microbiome.

57. The method of claim 51, wherein the synthetic microorganism is derived from a target microorganism isolated from the host microbiome.

58. The method of claim 56 or 57, wherein the target microorganism is susceptible to at least one antimicrobial agent.

59. The method of claim 58, wherein the target microorganism is selected from a bacterial, or fungal target microorganism.

60. The method of claim 59, wherein the target microorganism is a bacterial species capable of colonizing a dermal and/or mucosal niche and is a member of a genus selected from the group consisting of Staphylococcus, Streptococcus, Escherichia, Acinetobacter, Bacillus, Mycobacterium, Mycoplasma, Enterococcus, Corynebacterium, Klebsiella, Enterobacter, Trueperella, and Pseudomonas.

61. The method of claim 60, wherein the target microorganism is selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa, optionally wherein the target strain is a Staphylococcus aureus 502a strain or RN4220 strain.

62. The method of claim 54, wherein the synthetic microorganism kill switch molecular modification comprises a first cell death gene operably linked to a first regulatory region comprising a first inducible promoter.

63. The method of claim 62, wherein the first promoter is activated (induced) by a change in state in the microorganism environment in contradistinction to the normal physiological (niche) conditions at the at least one site in the subject.

64. The method of claim 63, wherein measurable average cell death of the synthetic microorganism occurs within at least a preset period of time following induction of the first promoter.

65. The method of claim 64, wherein the measurable average cell death occurs within at least a preset period of time selected from the group consisting of within at least 1, 5, 15, 30, 60, 90, 120, 180, 240, 300, or 360 min minutes following change of state.

66. The method of claim 65, wherein the measurable average cell death is at least a 50% cfu, at least 70%, at least 80%, at least 90%, at least 95%, at least 99%, at least 99.5%, at least 99.8%, or at least 99.9% cfu count reduction following the preset period of time.

67. The method of claim 63, wherein the change in state is selected from one or more of pH, temperature, osmotic pressure, osmolality, oxygen level, nutrient concentration, blood concentration, plasma concentration, serum concentration, interstitial fluid concentration, metal concentration, chelated metal concentration, change in composition or concentration of one or more immune factors, mineral concentration, and electrolyte concentration.

68. The method of claim 67, wherein the change in state is a higher concentration of and/or change in composition of blood, serum, plasma, or interstitial fluid compared to normal physiological (niche) conditions at the at least one site in the subject.

69. The method of claim 68, wherein the first promoter is a blood, serum, plasma, and/or heme responsive promoter.

70. The method of any one of claims 63 to 69, wherein the first promoter is upregulated by at least 1.5 fold, at least 3-fold, at least 5-fold, at least 10-fold, at least 20-fold, at least 50-fold, or at least 100-fold within a period of time selected from the group consisting of at least 30 min, 60 min, 90 min, 120 min, 180 min, 240 min, 300 min, and at least 360 min following the change of state.

71. The method of claim 70, wherein the first promoter is not induced, induced less than 1.5 fold, or is repressed in the absence of the change of state.

72. The method of claim 71, wherein the first promoter is induced at least 1.5, 2, 3, 4, 5 or at least 6 fold within a period of time in the presence of serum or blood.

73. The method of claim 72, wherein the first promoter is not induced, induced less than 1.5 fold, or repressed under the normal physiological (niche) conditions at the at least one site.

74. The method of claim 72, wherein the first promoter is not induced, induced less than 1.5 fold, or is repressed in the absence of blood, serum, plasma, or heme.

75. The method of any one of claim 62 to 74, wherein the synthetic microorganism is derived from a target microorganism that is a Staphylococcus aureus strain, and wherein the first promoter is derived from a gene selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

76. The method of claim 75, wherein the first promoter is derived from or comprises a nucleotide sequence selected from the group consisting of SEQ ID NO: 114, 115, 119, 120, 121, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 340, 341, 343, 345, 346, 348, 349, 350, 351, 352, 353, 359, 361, 363, 366, 370, or a substantially identical nucleotide sequence thereof.

77. The method of any one of claims 62 to 76, wherein the undesirable microorganism is a Staphylococcus aureus strain, and wherein the detectable presence is measured by a method comprising obtaining a sample from the at least one site of the subject, contacting a chromogenic agar with the sample, incubating the contacted agar and counting the positive cfus of the bacterial species after a predetermined period of time.

78. The method of any one of claims 62 to 77, wherein the cell death gene is selected from a toxin gene selected from the group consisting of sprA1, sprA2, kpn1, sma1, sprG, relF, rsaE, yoeB, mazF, yefM, and lysostaphin toxin gene.

79. The method of claim 78, wherein the cell death gene comprises a nucleotide sequence selected from the group consisting of SEQ ID NO: 122, 124, 125, 126, 127, 128, 274, 275, 284, 286, 288, 290, 315, and 317, or a substantially identical nucleotide sequence.

80. The method of any one of claims 62 to 79, wherein the synthetic microorganism further comprises an expression clamp molecular modification comprising an antitoxin gene specific for the first cell death gene, wherein the antitoxin gene is operably linked to a second regulatory region comprising a second promoter which is active upon dermal or mucosal colonization or in TSB media, but is repressed at least 2 fold upon exposure to blood, serum or plasma after a period of time selected from the group consisting of the group consisting of at least 30 min, 60 min, 90 min, 120 min, 180 min, 240 min, 300 min, and at least 360 min.

81. The method of claim 80, wherein the antitoxin gene encodes an antisense RNA sequence capable of hybridizing with at least a portion of the first cell death gene.

82. The method of claim 81, wherein the antitoxin gene is selected from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively.

83. The method of claim 82, wherein the antitoxin gene comprises a nucleotide sequence selected from the group consisting of SEQ ID NOs: 273, 306, 307, 308, 309, 310, 311, 312, 314, 319, 322, 342, 347, 362, 364, 368, 373, 374, 375, 376, 377, and 378 or a substantially identical nucleotide sequence

84. The method of any one of claims 80 to 83, wherein the second promoter is derived from a gene selected from the group consisting of clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR family transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

85. The method of claim 84, wherein the second promoter is a P_{clfB} (clumping factor B) and comprises a nucleotide sequence of SEQ ID NO: 117, 118, 129 or 130, or a substantially identical nucleotide sequence thereof.

86. The method of any one of claims 51 to 85, wherein the decolonizing step comprises topically administering a decolonizing agent to the at least one site in the subject to reduce or eliminate the presence of the undesirable microorganism from the at least one site.

87. The method of claim 86, wherein the decolonizing step comprises topical administration of the decolonizing agent, wherein no systemic antimicrobial agent is simultaneously administered.

88. The method of claim 86 or 87, wherein no systemic antimicrobial agent is administered within one week, two weeks, three weeks, one month, two months, three months, six months, or one year of the first topical administration of the decolonizing agent.

89. The method of any one of claims 86 to 88, wherein the decolonizing agent is selected from the group consisting of a disinfectant, bacteriocide, antiseptic, astringent, and antimicrobial agent.

90. The method of claim 89, wherein the decolonizing agent is selected from the group consisting of alcohols (ethyl alcohol, isopropyl alcohol), aldehydes (glutaraldehyde, formaldehyde, formaldehyde-releasing agents (noxythiolin=oxymethylenethiourea, tauroline, hexamine, dantoin), o-phthalaldehyde), anilides (triclocarban=TCC=3,4,4′-trichlorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), chloroxylenol (PCMX), 8-hydroxyquinoline, dodecyl benzene sulfonic acid, nisin, chlorine, glycerol monolaurate, C₈-C₁₄ fatty acids, quaternary ammonium compounds (cetrimide, benzalkonium chloride, cetyl pyridinium chloride), silver compounds (silver sulfadiazine, silver nitrate), peroxy compounds (hydrogen peroxide, peracetic acid, benzoyl peroxide), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), isotretinoin, sulfur compounds, botanical extracts (peppermint, calendula, eucalyptus, Melaleuca spp. (tea tree oil), (Vaccinium spp. (e.g., A-type proanthocyanidins), Cassia fistula Linn, Baekea frutescens L., Melia azedarach L., Muntingia calabura, Vitis vinifera L, Terminalia avicennioides Guill & Perr., Phylantus discoideus muel. Muel-Arg., Ocimum gratissimum Linn., Acalypha wilkesiana Muell-Arg., Hypericum pruinatum Boiss.&Bal., Hypericum olimpicum L. and Hypericum sabrum L., Hamamelis virginiana (witch hazel), Clove oil, Eucalyptus spp., Rosmarinus officinalis spp. (rosemary), thymus spp. (thyme), Lippia spp. (oregano), lemongrass spp., cinnamomum spp., geranium spp., lavendula spp., calendula spp.), aminolevulinic acid, topical antibiotic compounds (bacteriocins; mupirocin, bacitracin, neomycin, polymyxin B, gentamicin).

91. The method of claim 89 or 90, wherein the antimicrobial agent is selected from the group consisting of cephapirin, amoxicillin, trimethoprim-sulfonamides, sulfonamides, oxytetracycline, fluoroquinolones, enrofloxacin, danofloxacin, marbofloxacin, cefquinome, ceftiofur, streptomycin, oxytetracycline, vancomycin, cefazolin, cephalothin, cephalexin, linezolid, daptomycin, clindamycin, lincomycin, mupirocin, bacitracin, neomycin, polymyxin B, gentamicin, prulifloxacin, ulifloxacin, fidaxomicin, minocycline, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norfloxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, and fluconazole.

92. The method of any one of claims 86 to 91, wherein the decolonizing comprises topically administering the decolonizing agent at least one, two, three, four, five or six or more times prior to the replacing step.

93. The method of claim 92, wherein the decolonizing step comprises administering the decolonizing agent to the at least one host site in the subject from one to six or more times or two to four times at intervals of between 0.5 to 48 hours apart, and wherein the replacing step is performed after the final decolonizing step, optionally wherein the decolonizing agent is in the form of a spray, dip, lotion, cream, balm, or intramammary infusion.

94. The method of claim 93, wherein the replacing step comprises initial topical administration of a composition comprising at least 10⁵, at least 10⁶, at least 10⁷, at least 10⁸, at least 10⁹, at least 10¹⁰ CFU, or at least 10¹¹ of the synthetic strain and a pharmaceutically acceptable carrier to the at least one host site in the subject.

95. The method of claim 94, wherein the initial replacing step is performed within 12 hours, 24 hours, 36 hours, 2 days, 3 days, 4 days, 5 days, 6 days or 7 days of the decolonizing step.

96. The method of claim 94 or 95, wherein the replacing step is repeated at intervals of no more than once every two weeks to six months following the final decolonizing step.

97. The method of claim 94 or 95, wherein the decolonizing step and the replacing step is repeated at intervals of no more than once every two weeks to six months.

98. The method of any one of claims 94 to 97, wherein the replacing comprises administering the biotherapeutic composition comprising the synthetic microorganism to the at least one site at least one, two, three, four, five, six, seven, eight, nine, or ten times.

99. The method of claim 98, wherein the biotherapeutic composition is administered in the form of a spray, dip, lotion, cream, balm, or intramammary infusion.

100. The method of claim 98 or 99, wherein the replacing comprises administering the biotherapeutic composition comprising the synthetic microorganism to the at least one site no more than one, no more than two, no more than three times, or no more than four times per month.

101. The method of any one of claims 42 to 100, further comprising:
promoting colonization of the synthetic microorganism in the subject.

102. The method of claim 101, wherein the promoting colonization of the synthetic microorganism in the subject comprises administering to the subject a promoting agent, optionally where the promoting agent is a sealant, nutrient, prebiotic, commensal, stabilizing agent, emollient, humectant, and/or probiotic bacterial species.

103. The method of claim 102, wherein the promoting comprises administering from 10⁶ to 10¹⁰ cfu, or 10⁷ to 10⁹ cfu of the probiotic bacterial species to the subject after the initial decolonizing step.

104. The method of claim 102, wherein the nutrient is selected from sodium chloride, lithium chloride, sodium glycerophosphate, phenylethanol, mannitol, tryptone, peptide, and yeast extract.

105. The method of claim 102, wherein the prebiotic is selected from the group consisting of short-chain fatty acids (acetic acid, propionic acid, butyric acid, isobutyric acid, valeric acid, isovaleric acid), glycerol, pectin-derived oligosaccharides from agricultural by-products, fructo-oligosaccarides (e.g., inulin-like prebiotics), galacto-oligosaccharides (e.g., raffinose), succinic acid, lactic acid, and mannan-oligosaccharides.

106. The method of claim 102, wherein the probiotic is selected from the group consisting of Bifidobacterium breve, Bifidobacterium bifidum, Bifidobacterium lactis, Bifidobacterium infantis, Bifidobacterium breve, Bifidobacterium longum, Lactobacillus reuteri, Lactobacillus paracasei, Lactobacillus plantarum, Lactobacillus johnsonii, Lactobacillus rhamnosus, Lactobacillus acidophilus, Lactobacillus salivarius, Lactobacillus casei, Lactobacillus plantarum, Lactococcus lactis, Streptococcus thermophiles, and Enterococcus faecalis.

107. The method of claim 102, wherein the undesirable microorganism is selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa.

108. The method of any one of claims 42 to 107, wherein the undesirable microorganism is an antimicrobial agent-resistant microorganism.

109. The method of claim 108, wherein the antimicrobial agent-resistant microorganism is an antibiotic resistant bacteria.

110. The method of any one of claims 102 to 109, wherein the undesirable microorganism is a methicillin-resistant Staphylococcus aureus (MRSA) strain that contains a staphylococcal chromosome cassette (SCCmec types I-III), which encode one (SCCmec type I) or multiple antibiotic resistance genes (SCCmec type II and III), and/or produces a toxin.

111. The method of claim 110, wherein the toxin is selected from the group consisting of a Panton-Valentine leucocidin (PVL) toxin, toxic shock syndrome toxin-1 (TSST-1), staphylococcal alpha-hemolysin toxin, staphylococcal beta-hemolysin toxin, staphylococcal gamma-hemolysin toxin, staphylococcal delta-hemolysin toxin, enterotoxin A, enterotoxin B, enterotoxin C, enterotoxin D, enterotoxin E, and a coagulase toxin.

112. The method of any one of claims 42 to 111, wherein the subject does not exhibit recurrence of the undesirable microorganism at the at least one site for at least two weeks, at least two weeks, at least four weeks, at least six weeks, at least eight weeks, at least ten weeks, at least 12 weeks, at least 16 weeks, at least 24 weeks, at least 30 weeks, at least 36 weeks, at least 42 weeks, or at least 52 weeks after the administering step.

113. The method of any one of claims 42 to 112, wherein the biotherapeutic composition comprising a synthetic microorganism is administered pre-partum, early, mid-, or late lactation phase or in the dry period to the cow, goat or sheep in need thereof.

114. The method of any one of claims 42 to 113, wherein the subject is a bovine subject.

115. A method for treating and/or preventing mastitis or intramammary infection in a bovine, ovine, caprine, or porcine subject, comprising
(a) decolonizing the subject at at least one site; and
(b) recolonizing the subject at the at least one site with a live biotherapeutic composition according to any one of claims 1 to 41.

116. The method of any one of claims 43 to 115, wherein the at least one site includes one or more of teat canal, teat cistern, gland cistern, streak canal, teat apices, teat skin, udder skin, perineum skin, rectum, vagina, muzzle area, nares, and/or oral cavity of the subject.

117. The method of claim 115 or 116, wherein the somatic cell count (SCC) in milk from the subject is reduced within about 1, 2, or 3 weeks following first inoculation when compared to baseline pre-inoculation SCC, optionally wherein the SCC is reduced to no more than 300,000 cells/mL, no more than 200,000 cells/mL, or preferably no more than 150,000 cells/mL.

118. A kit comprising in at least one container the biotherapeutic composition comprising the synthetic microorganism according to any one of claims 1 to 41, and optionally one or more of at least a second container comprising a decolonizing agent, a sheet of instructions, at least a third container comprising a promoting agent, and/or an applicator.

119. A live biotherapeutic composition comprising
at least one synthetic microorganism, and a pharmaceutically acceptable carrier, wherein the synthetic microorganism comprises
a first molecular modification inserted to the genome of a target microorganism, the molecular modification comprising a first recombinant nucleotide comprising an action gene,
wherein the first recombinant nucleotide is operatively associated with an endogenous first regulatory region comprising a native inducible first promoter gene, and
wherein the native inducible first promoter imparts conditionally high level gene transcription of the first recombinant nucleotide in response to exposure to a change in state of at least three fold increase compared to basal productivity.

120. A live biotherapeutic composition comprising
at least one synthetic microorganism, and a pharmaceutically acceptable carrier, wherein the synthetic microorganism comprises
a first molecular modification inserted to the genome of a target microorganism, the molecular modification comprising a recombinant nucleotide comprising a first regulatory region comprising an inducible first promoter gene,
wherein the inducible first promoter gene is operably associated with an endogenous action gene, and
wherein the inducible first promoter imparts conditionally high level gene transcription of the endogenous action gene in response to a change in state of at least three fold increase of basal productivity.

121. The composition of claim 1, 119 or 120, wherein the basal productivity of the synthetic microorganism is determined by gene transcription level of the inducible first promoter gene and/or action gene or cell death gene when the synthetic microorganism is grown under a first environmental condition over a period of time.

122. The composition of claim 121, wherein the inducible first promoter gene of the synthetic microorganism is upregulated by at least 10-fold within a period of time of at least 120 min following the change in state comprising an exposure to a second environmental condition.

123. The composition of claim 119 or 120, wherein the target microorganism has the same genus and species as an undesirable microorganism.

124. The composition of claim 119 or 120, wherein the target microorganism is a wild-type microorganism or a synthetic microorganism.

125. The composition of claim 119 or 120, wherein the first promoter gene is not induced, induced less than 1.5 fold, or is repressed when the synthetic microorganism is grown under the first environmental condition.

126. The composition of claim 119, wherein the first recombinant gene further comprises a control arm immediately adjacent to the action gene.

127. The composition of claim 126, wherein the control arm includes a 5′ untranslated region (UTR) and/or a 3′ UTR relative to the action gene.

128. The composition of claim 126 or 127, wherein the control arm is complementary to an antisense oligonucleotide encoded by the genome of the synthetic microorganism.

129. The composition of claim 128, wherein the antisense oligonucleotide is encoded by a gene that is endogenous or inserted to the genome of the synthetic microorganism.

130. The composition of claim 119 or 120, wherein the first promoter gene induces conditionally high level gene expression of the action gene in response to exposure to the second environmental condition of at least three fold increase of basal productivity.

131. The composition of claim 119 or 120, wherein the action gene and the first promoter gene are within the same operon.

132. The composition of claim 131, wherein the action gene is integrated between the stop codon and the transcriptional terminator of any gene located in the same operon as the first promoter gene.

133. The composition of any one of claims 119 to 132, wherein the synthetic microorganism further comprises
at least a second molecular modification (expression clamp) comprising
a (anti-action) regulator gene encoding a small noncoding RNA (sRNA) specific for the control arm or action gene, wherein the regulator gene is operably associated with
an endogenous second regulatory region comprising a second promoter gene which is transcriptionally active (constitutive) when the synthetic microorganism is grown in the first environmental condition, but is not induced, induced less than 1.5-fold, or is repressed after exposure to the second environmental condition for a period of time of at least 120 minutes.

134. The composition of claim 133, wherein transcription of the regulator gene produces the sRNA in an effective amount to prevent or suppress the expression of the action gene when the microorganism is grown under the first environmental condition.

135. The synthetic microorganism of claim 119 or 120, wherein the first molecular modification is selected from the group consisting of kill switch molecular modification, virulence block molecular modification, metabolic molecular modification, and nano factory molecular modification.

136. The composition of claim 135, wherein the synthetic microorganism exhibits genomic stability of the first molecular modification and functional stability of the action gene over at least 500 generations.

137. The composition of claim 136, wherein the first molecular modification comprises a kill switch action gene including a first cell death gene operatively associated with the inducible first promoter gene.

138. The composition of claim 137, wherein the synthetic microorganism further comprises a deletion of at least a portion of a native action (toxin) gene.

139. The composition of claim 138, wherein the deletion of at least a portion of the native action (toxin) gene comprises a deletion of a native nucleic acid sequence selected from the group consisting of the Shine-Dalgarno sequence, ribosomal binding site, and the transcription start site of the native toxin gene.

140. The composition of claim 138 or 139, wherein the synthetic microorganism further comprises a deletion of at least a portion of a native antitoxin gene specific for the native toxin gene.

141. The composition of claim 140, wherein the native antitoxin gene encodes an mRNA antisense or antitoxin peptide specific for the native toxin gene, mRNA or toxin encoded thereby.

142. The composition of any one of claims 137 to 141, wherein a measurable average cell death of the synthetic microorganism occurs within at least a preset period of time following change of state when the synthetic microorganism is exposed to the second environmental condition.

143. The composition of claim 142, wherein the measurable average cell death occurs within at least a preset period of time selected from the group consisting of within at least 1, 5, 15, 30, 60, 90, 120, 180, 240, 300, or 360 min minutes following exposure to the second environmental condition.

144. The composition of claim 143, wherein the measurable average cell death is a cfu count reduction of at least 50%, at least 70%, at least 80%, at least 90%, at least 95%, at least 99%, at least 99.5%, at least 99.8%, or at least 99.9% cfu count reduction following the preset period of time.

145. The composition of any one of claims 135 to 144, wherein the kill switch molecular modification reduces or prevents infectious growth of the synthetic microorganism within the second environmental condition.

146. The composition of any one of claims 119 to 145, wherein the first environmental condition is selected from the group consisting of dermal, mucosal, genitourinary, gastrointestinal, or a complete media.

147. The composition of any one of claims 119 to 146, wherein the second environmental condition comprises exposure to or an increase in concentration of blood, plasma, serum, interstitial fluid, synovial fluid, contaminated cerebral spinal fluid, or lactose.

148. The composition of any one of claims 119 to 147, wherein the target microorganism is susceptible to at least one antimicrobial agent.

149. The composition of any one of claims 119 to 148, wherein the target microorganism is selected from the group consisting of bacteria and yeast target microorganisms.

150. The composition of claim 149, wherein the target microorganism is a bacterial species having a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

151. The composition of claim 150, wherein the target microorganism is selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcus hyicus, Acinetobacter baumannii, Acinetobacter calcoaceticus, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa, optionally wherein the target strain is a Staphylococcus aureus 502a strain or RN4220 strain.

152. The composition of claim 150 or 151, wherein the target microorganism is selected from the group consisting of Staphylococcus aureus, Escherichia coli, and Streptococcus spp.

153. The composition of any one of claims 119 to 152, comprising a mixture of synthetic microorganisms prepared from each of a Staphylococcus aureus, a Escherichia coli, and a Streptococcus agalactiae target strain.

154. The composition of any one of claims 150 to 153, wherein the action gene is a cell death gene selected from or derived from the group consisting of sprA1, sprA2, sprG, mazF, relE, relF, hokB, hokD, yafQ, rsaE, yoeB, yefM, kpn1, sma1, or lysostaphin toxin gene.

155. The composition of claim 154, wherein the cell death gene comprises a nucleotide sequence selected from the group consisting of SEQ ID NOs: BP_DNA_003 (SEQ ID NO: 342), BP_DNA_008 (SEQ ID NO: 347), BP_DNA_0032 (SEQ ID NO: 362), BP_DNA_035 (SEQ ID NO:364), BP_DNA_045 (SEQ ID NO: 368), BP_DNA_065 (SEQ ID NO: 373), BP_DNA_067 (SEQ ID NO: 374), BP_DNA_068 (SEQ ID NO: 375), BP_DNA_069 (SEQ ID NO: 376), BP_DNA_070 (SEQ ID NO: 377), BP_DNA_071 (SEQ ID NO: 378), or a substantially identical nucleotide sequence.

156. The composition of any one of claims 150 to 155, wherein the cell death gene encodes a toxin peptide or protein comprising an amino acid sequence of SEQ ID NO: 104, 105, 106, 107, 108, 109, 110, 111, 112, 285, 287, 289, 291, 305, 316, 318, 321, 411, 423, 596, or a substantially similar amino acid sequence

157. The composition of any one of claims 150 to 156, wherein the target microorganism is a S. aureus strain, and the inducible first promoter gene is selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosynthesis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrochrome transport ATP-binding protein fhuA), fhuB (ferrochrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

158. The composition of claim 157, wherein the inducible first promoter gene comprises a nucleotide sequence complementary to an upstream or downstream homology arm having a nucleic acid sequence selected from the group consisting of BP_DNA_001 (SEQ ID NO: 340), BP_DNA_002 (SEQ ID NO: 341), BP_DNA_004 (SEQ ID NO: 343), BP_DNA_006 (SEQ ID NO: 345), BP_DNA_007 (SEQ ID NO: 346), BP_DNA_010 (SEQ ID NO: 348), BP_DNA_BP_DNA_012 (SEQ ID NO: 349), BP_DNA_013 (SEQ ID NO: 350), BP_DNA_014 (SEQ ID NO: 351), BP_DNA_016 (SEQ ID NO: 352), BP_DNA_017 (SEQ ID NO: 353), BP_DNA_029 (SEQ ID NO: 359), BP_DNA_031 (SEQ ID NO: 361), BP_DNA_033 (SEQ ID NO: 363), BP_DNA_041 (SEQ ID NO: 366), and BP_DNA_057 (SEQ ID NO: 370), or a substantially identical nucleotide sequence thereof.

159. The composition of any one of claims 119 to 158, wherein the synthetic microorganism comprises a second molecular modification encoding an sRNA sequence capable of hybridizing with at least a portion of the action gene, or encoding an peptide specific for at least a portion of a protein encoded by the action gene.

160. The composition of claim 159, wherein the second molecular modification comprises or is derived from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively.

161. The composition of claim 159, wherein the second molecular modification comprises a nucleotide sequence comprising BP_DNA_005 (SEQ ID NO: 344), or a substantially identical nucleotide sequence.

162. The composition of any one of claims 158 to 161, wherein the second promoter comprises or is derived from a gene selected from the group consisting of PsprA1as (sprA1as native promoter), clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR family transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

163. The composition of any one of claims 119 to 162, wherein the pharmaceutically acceptable carrier includes an excipient, diluent, emollient, binder, lubricant, sweetening agent, flavoring agent, wetting agent, preservative, buffer, or absorbent, or a combination thereof.

164. The composition of claim 163, further comprising a nutrient, prebiotic, commensal, and/or probiotic bacterial species.

165. A single dose unit comprising the composition of any one of claims 119 to 164.

166. The single dose unit of claim 165, comprising at least at least 10⁵, at least 10⁶, at least 10⁷, at least 10⁸, at least 10⁹, at least 10¹⁰ CFU, or at least 10¹¹ of the synthetic microorganism and a pharmaceutically acceptable excipient.

167. The dose unit of claim 166, formulated for topical administration.

168. The composition of any one of claims 119 to 164 or single dose unit of any one of claims 165 to 167 for use in the manufacture of a medicament for eliminating and preventing the recurrence of a undesirable microorganism in a subject.

169. The composition of any one of claims 119 to 164 or single dose unit of any one of claims 165 to 167, for use in treatment or prevention of a skin and soft tissue infection (SSTI) or bacteremia in a subject.

170. The composition of claim 169, wherein the SSTI is mastitis and/or intramammary infection.

171. The composition of claim 169, wherein the subject is selected from the group consisting of a bovine, caprine, ovine, porcine, and human subject.
